(19) United States
(12) Patent Application Publication
(10) Pub. No.: US 2022/0298138 A1
(43) Pub. Date: Sep. 22, 2022

(54) ENZYME INHIBITORS

(71) Applicant: KALVISTA PHARMACEUTICALS LIMITED, Salisbury (GB)

(72) Inventors: Rebecca Louise DAVIE, Salisbury (GB); Hannah Joy EDWARDS, Salisbury (GB); David Michael EVANS, Salisbury (GB); Simon Teanby HODGSON, Ampthill (GB); Sally Louise MARSH, Salisbury (GB); Alessandro MAZZACANI, Salisbury (GB); David Philip ROOKER, Salisbury (GB); Michael John STOCKS, Leicestershire (GB); Andrew Peter CRIDLAND, Harlow Essex (GB); Emanuela GANCIA, Harlow Essex (GB); Julie Nicole HAMBLIN, Saffron Walden Essex (GB); Paul Stuart HINCHLIFFE, Saffron Walden Essex (GB); Stefano LEVANTO, Saffron Walden Essex (GB); Terence Aaron PANCHAL, Harlow Essex (GB); Iain Robert MILLER, Nottingham (GB)

(21) Appl. No.: 17/633,980

(22) PCT Filed: Aug. 21, 2019
(86) PCT No.: PCT/GB2019/052356

§ 371 Date: Feb. 9, 2022

(51) Int. Cl. C07D 401/12; C07D 217/22; C07D 413/12; C07D 401/14; C07D 471/04; C07D 417/12; C07D 487/04; C07D 405/12; C07D 409/12; C07D 495/04; C07D 209/08; C07D 231/12; C07D 213/73; C07C 233/37; C07D 249/06; C07D 265/36; C07D 333/58; C07D 403/12; C07D 403/14

(52) U.S. Cl. CPC C07D 401/12; C07D 217/22; C07D 413/12; C07D 401/14; C07D 471/04; C07D 417/12; C07D 487/04; C07D 405/12; C07D 409/12; C07D 495/04; C07D 209/08; C07D 231/12; C07D 213/73; C07C 233/37; C07D 249/06; C07D 265/36; C07D 333/58; C07D 403/12; C07D 403/14

(57) ABSTRACT

The present invention provides compounds of formula (I): Formula (I) compositions comprising such compounds; the use of such compounds in therapy; and methods of treating patients with such compounds; wherein A, Y, n, R1, R2^{A}, R2^{B}, R3 and *1 are as defined herein.

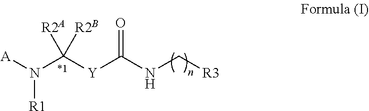

This invention relates to enzyme inhibitors that are inhibitors of Factor XIIa (FXIIa), and to the pharmaceutical compositions, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16; 129 (11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa.

FXIIa has a unique and specific structure that is different from many other serine proteases. For instance, the Tyr99 in FXIIa points towards the active site, partially blocking the 52 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst. 2019, D75, 1-14; “Structures of human plasma 1-factor XIIa cocrystallized with potent inhibitors” A Dementiev et al., Blood Advances 2018, 2 (5), 549-558; “Design of Small-Molecule Active-Site Inhibitors of the S1A Family Proteases as Procoagulant and Anticoagulant Drugs” P. M. Fischer, J. Med. Chem., 2018, 61 (9), 3799-3822; “Assessment of the protein interaction between coagulation factor XII and corn trypsin inhibitor by molecular docking and biochemical validation” B. K. Hamad et al. Journal of Thrombosis and Haemostasis, 15: 1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Gobel et al., Proc Natl Acad Sci USA. 2019 Jan. 2; 116 (1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285 (45):35206-15. doi: 10.1074/jbc.M110.171769). FXIIa activation of plasminogen to plasmin contributes to fibrinolysis (see Konings et al., Thromb Res. 2015 August; 136 (2):474-80. doi: 10.1016/j.thromres.2015.06.028). PKa proteolytically cleaves NPY and thereby alters its binding to NPY receptors (Abid et al., J Biol Chem. 2009 Sep. 11; 284 (37):24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B978-0-12-800100-4.00002-7; and Hopp et al., J Neuroinflammation. 2017 Feb. 20; 14 (1):39. doi: 10.1186/s12974-017-0815-8). CSL-312, an antibody inhibitory against FXIIa, is currently in clinical trials for the prophylactic prevention and treatment of both C1 inhibitor deficient and normal C1 inhibitor hereditary angioedema (HAE), which results in intermittent swelling of face, hands, throat, gastro-intestinal tract and genitals (see https://www.clinicaltrials.gov/ct2/show/NCT03712228). Mutations in FXII that facilitate its activation to FXIIa have been identified as a cause of HAE (see Björkqvist et al., J Clin Invest. 2015 Aug. 3; 125 (8):3132-46. doi: 10.1172/JC177139; and de Maat et al., J Allergy Clin Immunol. 2016 November; 138 (5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17 (1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 Apr. 30:194599819846446. doi: 10.1177/0194599819846446) and silver nanoparticles such as those used as antibacterial components in healthcare, biomedical and consumer products (Long et al., Nanotoxicology. 2016; 10 (4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simão et al., Blood. 2017 Apr. 20; 129 (16):2280-2290. doi: 10.1182/blood-2016-09-740670; Fröhlich et al., Stroke. 2019 Jun. 11:STROKEAHA119025260. doi: 10.1161/STROKEAHA.119.025260; Rathbun, Oxf Med Case Reports. 2019 Jan. 24; 2019 (1):omy112. doi: 10.1093/omcr/omy112; Lekoubou et al., Neurol Res. 2014 July; 36 (7):687-94. doi: 10.1179/1743132813Y.0000000302; Hill et al., Neurology. 2003 May 13; 60 (9):1525-7).

Stone et al. (Immunol Allergy Clin North Am. 2017 August; 37 (3):483-495.) reports that certain drugs can cause angioedema.

Scott et al. (Curr Diabetes Rev. 2018; 14 (4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124 (1):115-122. doi: 10.1111/bcpt.13097) reports angiotensin II receptor blocker (ARB)-related angioedema. Reichman et al., (Pharmacoepidemiol Drug Saf. 2017 October; 26 (10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28 (5):e44-e45. doi: 10.1016/j.jstrokecerebrovasdis.2019.01.030) also reports a possible association between certain angioedemas and ARBs.

Giard et al. (Dermatology. 2012; 225 (1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394 (3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13 (2):181-8. Epub 2007 Jan. 28 and Gao et al., J Proteome Res. 2008 June; 7 (6):2516-25. doi: 10.1021/pr800112 g). FXIIa has been implicated in mediating both vascular endothelial growth factor (VEGF) independent DME (see Kita et al., Diabetes. 2015 October; 64 (10):3588-99. doi: 10.2337/db15-0317) and VEGF mediated DME (see Clermont et al., Invest Ophthalmol Vis Sci. 2016 May 1; 57 (6):2390-9. doi: 10.1167/iovs.15-18272). FXII deficiency is protective against VEGF induced retinal edema in mice (Clermont et al., ARVO talk 2019).

Therefore it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1; 140 (3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 Mar. 31; 112 (13):4068-73. doi: 10.1073/pnas.1423764112; Simões et al., J Neurochem. 2019 August; 150 (3):296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135 (4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18; 145 (3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50 (4):611-7; and Puy et al., J Thromb Haemost. 2013 July; 11 (7):1341-52. doi: 10.1111/jth.12295). Studies on both humans and experimental animal models have demonstrated that FXII deficiency prolongs activated partial prothrombin time (APTT) without adversely affecting hemostasis (see Renn6 et al., J Exp Med. 2005 Jul. 18; 202 (2):271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3 (17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360 (3):466-475. doi: 10.1124/jpet.116.238493; Krupka et al., PLoS One. 2016 Jan. 27; 11 (1):e0146783. doi: 10.1371/journal.pone.0146783; Leung et al., Transl Stroke Res. 2012 September; 3 (3):381-9. doi: 10.1007/s12975-012-0186-5; Simão et al., Blood. 2017 Apr. 20; 129 (16):2280-2290. doi: 10.1182/blood-2016-09-740670; and Liu et al., Nat Med. 2011 February; 17 (2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274 (1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10 (2):94-105. doi: 10.1159/000484257; and Ghebrehiwet et al., J Exp Med. 1981 Mar. 1; 153 (3):665-76).

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

However, there remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC1 Inh, ACE and tPA induced angioedema; vascular hyperpermeability; stroke including ischemic stroke and haemorrhagic accidents; retinal edema; diabetic retinopathy; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis. In particular, there remains a need to develop new FXIIa inhibitors.

### DESCRIPTION OF THE INVENTION

The present invention relates to a series of amide compounds that are inhibitors of factor XIIa (FXIIa). These compounds of the invention are potentially useful in the treatment of diseases or conditions in which factor XIIa is implicated. The invention further relates to pharmaceutical compositions of the inhibitors, to the use of the compositions as therapeutic agents, and to methods of treatment using these compositions.

In a first aspect, the present invention provides compounds of formula (I):

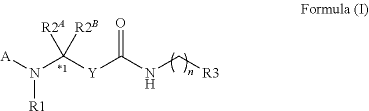

wherein
*1 denotes a chiral centre
n=0, 1 or 2;
A is selected from H, —(C═O)R4, —SO₂R6, and —(CH₂)—R13;
Y is either a bond, or —[CHR5]-;
R1 is H or alkyl^{b};
R2^{A} is selected from H, alkyl, —(CH₂)₀₋₃aryl, —(CH₂)₀₋₃heteroaryl, —(CH₂)₀₋₃cycloalkyl,
—(CH₂)₀₋₃-[benzothiophene], —(CH₂)₀₋₃-[indole], and;

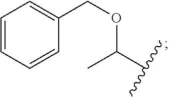 [SMILES: *C(C)OCc1ccccc1]

or, when Y is a bond, R1 and R2^{A}, together with nitrogen atom to which R1 is attached and the carbon atom to which R2^{A} is attached, may be linked by alkylene to form a 4-, 5-, or 6-membered saturated heterocycle, optionally wherein the 4-, 5-, or 6-membered saturated heterocycle may be substituted with aryl, or wherein two adjacent carbon atoms on the 4-, 5-, or 6-membered saturated heterocycle may be linked to form a 6-membered aromatic ring, or wherein two adjacent carbon atoms on the 4-, 5-, or 6-membered saturated heterocycle may be linked to form a 3-, 4-, or 5-membered saturated hydrocarbon ring which may be optionally mono- or di-substituted by alkyl^{b};
when Y is —[CHR5]-, R5 is H; or,
when Y is —[CHR5]-, together with the carbon atoms to which each of R5 and R2^{A} are attached, R5 and R2^{A} may be linked by alkylene to form a 4-, 5-, 6-membered saturated ring; or,
when Y is —[CHR5]-, together with the nitrogen atom to which R1 is attached, the carbon atom to which R5 is attached, and the carbon atom to which R2^{A} and R2^{B} are both attached, R5 and R1 may be linked by alkylene to form a saturated 4-, 5-, or 6-membered heterocycle, optionally wherein one atom on the saturated 4-, 5-, or 6-membered heterocycle may be linked by alkylene to join with R2^{A};
R2^{B} is H or alkyl^{b}; or,
R2^{A} and R2^{B}, together with the carbon to which R2^{A} and R2^{B} are both attached, may be linked by alkylene or heteroalkylene to form a 3-, 4-, 5-, or 6-membered saturated ring, optionally wherein the 3-, 4-, 5-, or 6-membered saturated ring contains one or two ring members that are selected from N and O;
R3 is:
(i) a fused 6,5- or 6,6-bicyclic ring, containing one heteroatom selected from S and N, wherein at least one of the rings is aromatic and, optionally the bicyclic ring contains one additional heteroatom independently selected from N, O and S;
   optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring; or
(ii) phenyl, pyridyl, or thiophenyl, which may be optionally substituted with 1, 2 or 3 substituents independently selected from alkyl^{b}, alkoxy, OH, NH₂ halo, CN, CF₃, —C(═NH)NH₂, and heteroaryl^{b};
   wherein when n=1, and R3 is phenyl substituted with at least one —(CH₂NH₂), R2^{A} is alkyl and R2^{B} is H; or

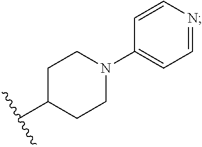 [SMILES: *C1CCN(c2ccncc2)CC1]

R4 is one of:
(i) a group of formula (II),

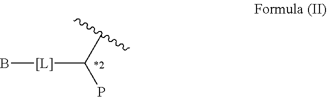

wherein -[L]- is a bond, —[(CH₂)₁₋₄]—, —[(CH₂)—O—(CH₂)]—, or —[O—(CH₂)]—; and P is alkoxy, OH or NR11R12;
wherein *2 denotes a chiral centre, and
wherein when -[L]- is a bond, B is a C₁₋₄ linear or branched chain hydrocarbon, and
wherein when -[L]- is —[(CH₂)₁₋₄]—, —[(CH₂)—O—(CH₂)]—, or —[O—(CH₂)]—, B is OH, aryl, heteroaryl, heterocyclyl, cycloalkyl or;

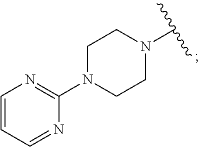 [SMILES: *N1CCN(c2ncccn2)CC1]

or,
(ii) —(CH₂)ₘ-[fused 6,5- or 6,6-heteroaromatic bicyclic ring], wherein at least one ring atom is a heteroatom selected from O, N or S, and optionally, 1, 2 or 3 additional ring atoms may be selected from N or NH; wherein the fused 6,5- or 6,6-heteroaromatic bicyclic ring may be optionally substituted with 1, 2 or 3 substituents independently selected from alkyl^{b}; wherein the 6,5-heteroaromatic bicyclic ring may be attached to —(CH₂)ₘ— via the 6- or 5-membered ring; or, (iii) methyl, —C(CH₃)₂(OH), —C(CH₃)₂(NHMe), —(CH₂)ₘ-(aryl), —(CH₂)ₘ-(cycloalkyl), —(CH₂)ₘ-(heteroaryl), —(CH₂)ₘ-(heterocyclyl), —(CH₂)-(alkyl), —(CH(halo)₂), —(CH₂)ₘ—(NR8R9), —(CH₂)ₘ—(NR10R7), —(CH₂)ₘ—O—(CH₂)ₖ-(aryl), —(CH₂)ₘ—(SO₂)—(CH₂)ₖ-(aryl), —(CH₂)ₘ-(alkoxy), —(CH₂)ₘ—O—(CH₂)ₖ-(heteroaryl), or —(CH₂)ₘ-[pyridone, which may be optionally substituted by alkyl^{b}, or CF₃];
wherein k=0, 1, 2, or 3;
wherein m=0, 1, 2 or 3;
wherein:
   when Y is —[CHR5]- and R5 is H, R2^{A} is CH₂-aryl or H; and
   when Y is —[CHR5]-, R3 is;

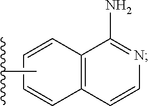

when A is H, R3 is;

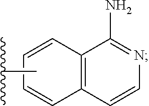

and when R3 is

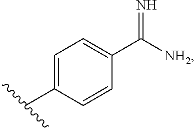 [SMILES: *c1ccc(C(=N)N)cc1]

R2^{A} is not H;
wherein:
R6 is alkyl or —(CH₂)₀₋₃-(aryl);
R7 is independently selected from H, —SO₂CH₃, methyl, ethyl, propyl, isopropyl, and cycloalkyl;
R8 and R9 are independently selected from H, —SO₂CH₃, alkyl^{b}, heteroaryl^{b}, and cycloalkyl; or R8 and R9 together with the nitrogen atom to which they are attached form a carbon-containing 4-, 5-, 6- or 7-membered heterocyclic ring, optionally containing an additional heteroatom selected from N, NR10, S, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃; or R8 and R9 together with the nitrogen atom to which they are attached form a carbon-containing 5- or 6-membered heterocyclic ring, which is fused to an aryl^{b} or a heteroaryl^{b};
R10 is independently selected from H, —SO₂R6, alkyl^{b}, —(CH₂)₀₋₃aryl^{b}, —(CH₂)₀₋₃heteroaryl^{b}, cycloalkyl, —(C═O)-(aryl), and —(CH₂)₀₋₃heterocyclyl^{b}; or R10 is a carbon-containing 4-, 5-, 6- or 7-membered heterocyclic ring, optionally containing an additional heteroatom selected from N, NR7, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃;
R11 and R12 are independently selected from H, alkyl^{b}, —SO₂R6, cycloalkyl, —(C═O)O-(alkyl^{b}), —(C═O)-phenyl, —CH₂-phenyl, and CH₂—COOH; or R11 and R12 together with the nitrogen atom to which they are attached form a carbon-containing 4-, 5-, 6- or 7-membered heterocyclic ring optionally containing an additional heteroatom selected from N, O, and NR10, wherein the heterocyclic ring may be optionally mono- or di-substituted with substituents independently selected from alkyl^{b}, OH, halo and CF₃;
R13 is selected from heteroaryl, cycloalkyl, heterocyclyl and aryl^{b};
wherein:
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R7)₂ and fluoro;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₁-C₆)alkoxy, OH, —NR8R9, —NHCOCH₃, —CO(heterocyclyl^{b}), —COOR8, —CONR8R9, CN, CF₃, halo, oxo and heterocyclyl^{b};
alkyl^{b} is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₁-C₆)alkoxy, OH, —N(R7)₂, —NHCOCH₃, CF₃, halo, oxo and cyclopropane;
alkylene is a bivalent linear saturated hydrocarbon having 1 to 5 carbon atoms (C₁₋C₅); alkylene may optionally be substituted with 1 or 2 substituents independently selected from alkyl, (C₁-C₆)alkoxy, OH, CN, CF₃ and halo;
aryl is phenyl, biphenyl or naphthyl; aryl may be optionally substituted with 1, 2 or 3 substituents independently selected from alkyl, alkoxy, OH, —SO₂CH₃, halo, —SO₂NR8R9, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR8, —CONR8R9, —(CH₂)₀₋₃—NR8R9, OCF₃ and CF₃; or two adjacent carbon ring atoms on the aryl may be optionally linked by a heteroalkylene to form a non-aromatic ring containing 5, 6, or 7 ring members which may be optionally substituted with OH; or optionally wherein two adjacent ring atoms on aryl are linked to form a 5- or 6-membered aromatic ring containing 1 or 2 heteroatoms that are selected from N, NR10, S, and O;
aryl^{b} is phenyl, biphenyl or naphthyl, which may be optionally substituted with 1, 2 or 3 substituents independently selected from methyl, ethyl, propyl, isopropyl, alkoxy, OH, —SO₂CH₃, N(R7)₂, halo, CN, and CF₃; or two adjacent carbon ring atoms on the aryl may be optionally linked by a heteroalkylene to form a non-aromatic ring containing 5, 6, or 7 ring members;
cycloalkyl is monocyclic saturated hydrocarbon ring of between 3 and 6 carbon atoms (C3-C₆); cycloalkyl may optionally be substituted with 1 or 2 substituents independently selected from methyl, ethyl, propyl, isopropyl, methoxy, ethoxy, propoxy, isopropoxy, OH, CN, CF₃ and halo; optionally wherein two adjacent ring atoms on cycloalkyl are linked to form a 5- or 6-membered saturated hydrocarbon ring;
halo is F, Cl, Br, or I;
heteroalkylene is a bivalent linear saturated hydrocarbon having 2 to 5 carbon atoms (C₂-C₅), wherein 1 or 2 of the 2 to 5 carbon atoms are replaced with NR10, S, or O; heteroalkylene may optionally be substituted with 1 or 2 substituents independently selected from alkyl, (C₁-C₆)alkoxy, OH, CN, CF₃ and halo;
heteroaryl is a 5- or 6-membered carbon-containing aromatic ring containing one, two or three ring members that are selected from N, NR10, S, and O; heteroaryl may be optionally substituted with 1, 2 or 3 substituents independently selected from alkyl, alkoxy, heteroaryl^{b}, phenyl, cycloalkyl, OH, OCF₃, halo, heterocyclyl^{b}, CN, and CF₃;
heteroaryl^{b} is a 5- or 6-membered carbon-containing aromatic ring containing one, two or three ring members that are selected from N, NR10, S, and O; heteroaryl^{b} may be optionally substituted with 1, 2 or 3 substituents independently selected from methyl, ethyl, propyl, isopropyl, alkoxy, OH, OCF₃, COOCH₃, COOCH₂CH₃, COO—(CH₂)₂—CH₃, COO-(iPr), halo, CN, and CF₃;
heterocyclyl is a 4-, 5-, 6-, or 7-membered carbon-containing non-aromatic ring containing one, two, three, or four ring members that are selected from N, NR10, S, SO, SO₂ and O; heterocyclyl may be optionally substituted with 1, 2, 3, or 4 substituents independently selected from alkyl, alkoxy, aryl^{b}, OH, OCF₃, halo, oxo, CN, NR8R9, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two ring atoms on heterocyclyl are linked with an heteroalkylene to form a non-aromatic ring containing 5, 6, or 7 ring members; or optionally wherein two adjacent ring atoms on heterocyclyl are linked to form a 5- or 6-membered aromatic ring which may optionally contain 1 or 2 heteroatoms that are selected from N, NR10, S, and O;
heterocyclyl^{b} is a 4-, 5-, 6-, or 7-membered carbon-containing non-aromatic ring containing one, two or three ring members that are selected from N, NR7, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃;
   and tautomers, isomers, stereoisomers (including enantiomers, diastereoisomers and racemic and scalemic mixtures thereof), deuterated isotopes, and pharmaceutically acceptable salts and/or solvates thereof.

The invention is also described by the appended numbered embodiments.

The compounds of the present invention have been developed to be inhibitors of FXIIa. As noted above, FXIIa has a unique and specific binding site and there is a need for small molecule FXIIa inhibitors.

The present invention also provides a prodrug of a compound of formula (I) as herein defined, or a pharmaceutically acceptable salt and/or solvate thereof.

The present invention also provides an N-oxide of a compound of formula (I) as herein defined, or a prodrug or pharmaceutically acceptable salt and/or solvate thereof.

It will be understood that certain compounds of the present invention may exist in solvated, for example hydrated, as well as unsolvated forms. It is to be understood that the present invention encompasses all such solvated forms.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, R2^{A} can be —(CH₂)₀₋₃(aryl), which more specifically can be phenyl. In this case, phenyl can be optionally substituted in the same manner as “heterocyclyl”.

It will be understood that “alkylene” has two free valencies i.e. it is bivalent, meaning that it is capable of being bonded to twice. For example, when R2^{A} and R2^{B}, together with the carbon to which R2^{A} and R2^{B} are both attached, are linked by alkylene to form a 4-, membered saturated ring, the alkylene will be —CH₂CH₂CH₂—.

It will be understood that *X indicates a chiral centre, i.e. one where the stereochemistry is fixed in either the (R)-, or (S)-configuration about the atom to which is indicated.

As noted above, heteroalkylene is a bivalent linear saturated hydrocarbon having 2 to 5 carbon atoms (C₂-C₅), wherein 1 or 2 of the 2 to 5 carbon atoms are replaced with NR10, S, or O; wherein heteroalkylene may optionally be substituted with 1 or 2 substituents independently selected from alkyl, (C₁-C₆)alkoxy, OH, CN, CF₃ and halo. It will be understood that, for example, —CH₂O— is a “heteroalkylene” having 2 carbon atoms wherein one of the 2 carbon atoms has been replaced with 0.

The term “O-linked”, such as in “O-linked hydrocarbon”, means that the hydrocarbon is joined to the remainder of the molecule via an oxygen atom.

The term “N-linked”, such as in “N-linked pyrrolidinyl”, means that the pyrrolidinyl group is joined to the remainder of the molecule via a ring nitrogen atom.

It will be understood that when any variable occurs more than once, its definition on each occurrence is independent of every other occurrence.

It will be understood that combinations of substituents and variables are permissible only if such combinations result in stable compounds.

As is clear from the definitions above, and for the avoidance of any doubt, it will be understood that “B”, “P” and “Y” define closed groups as defined above, and do not encompass Boron, Phosphate and Yttrium, respectively.

It will be understood that lines drawn into any ring systems from substituents represents that the indicated bond can be attached to any of the substitutable ring atoms. For example the following structure indicates that the point of attachment can be from any of the substitutable ring atoms on ring system:

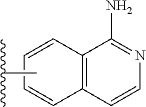

It will be understood that linking moieties (such as -[L]-) will be read from left to right, as indicated in their definition. For example, when R4 is a group of formula (II):

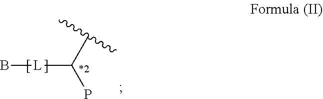

and -[L]- is —[O—(CH₂)]—, then Formula (II) should be read only as:

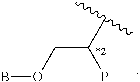

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

As noted above, n can be 0.

When n is 0, R3 can be

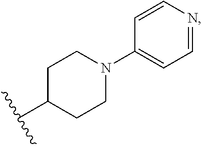 [SMILES: *C1CCN(c2ccncc2)CC1]

Y can be a bond, R2^{A} can be aryl, A can be —(C═O)R4 and R4 can be (CH₂)—(NR8R9). Specifically R2^{A} can be phenyl substituted by at least one halo, in particular fluoro, in particular two fluoro substituents. Specifically, NR8R9 can be

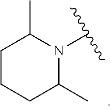 [SMILES: *N1C(C)CCCC1C]

Alternatively, when n is 0, R3 can be

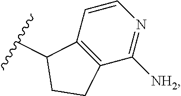 [SMILES: *C1CCc2c1ccnc2N]

Y can be a bond, R2^{A} can be alkyl, preferably methyl, A can be —(C═O)R4, and R4 can be a group of formula (II). In particular, B can be phenyl, P can be NH₂ and -[L]- can be —[(CH₂)₂]—.

As noted above, n can also be 1. Preferably, n is 1.

When n is 1, A can be selected from H, —(C═O)R4, —SO₂R6, and —(CH₂)—R13. Preferably, A is selected from —(C═O)R4 and —SO₂R6. More preferably, A is —(C═O)R4.

When A is —(C═O)R4, R4 can be one of:
(i) a group of formula (II),

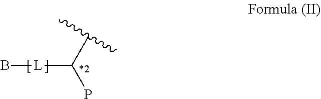

wherein -[L]- can be a bond, —[(CH₂)₁₋₄]—, —[(CH₂)—O—(CH₂)]—, or —[O—(CH₂)]—; and P can be alkoxy, OH or NR11R12;
wherein *2 denotes a chiral centre
wherein when -[L]- is a bond, B is a C₁₋₄ linear or branched chain hydrocarbon, and
wherein when -[L]- is —[(CH₂)₁₋₄]—, —[(CH₂)—O—(CH₂)]—, or —[O—(CH₂)]—, B is OH, aryl, heteroaryl, heterocyclyl, cycloalkyl

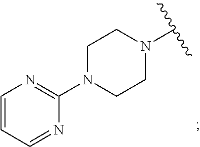 [SMILES: *N1CCN(c2ncccn2)CC1]

or,
(ii) —(CH₂)ₘ-[fused 6,5- or 6,6-heteroaromatic bicyclic ring], wherein at least one ring atom can be a heteroatom selected from O, N or S, and optionally, 1, 2 or 3 additional ring atoms can be selected from N or NH; wherein the fused 6,5- or 6,6-heteroaromatic bicyclic ring can be optionally substituted with 1, 2 or 3 substituents independently selected from alkyl^{b}; wherein the 6,5-heteroaromatic bicyclic ring can be attached to —(CH₂)ₘ— via the 6- or 5-membered ring; or,
(iii) methyl, —C(CH₃)₂(OH), —C(CH₃)₂(NHMe), —(CH₂)ₘ-(aryl), —(CH₂)ₘ-(cycloalkyl), —(CH₂)ₘ-(heteroaryl), —(CH₂)ₘ-(heterocyclyl), —(CH₂)-(alkyl), —(CH(halo)₂), —(CH₂)ₘ—(NR8R9), —(CH₂)ₘ—(NR10R7), —(CH₂)ₘ—O—(CH₂)ₖ-(aryl), —(CH₂)ₘ—(SO₂)—(CH₂)ₖ- (aryl), —(CH₂)ₘ-(alkoxy), —(CH₂)ₘ—O—(CH₂)ₖ-(heteroaryl), or —(CH₂)ₘ-[pyridone, which may be optionally substituted by alkyl^{b}, or CF₃];
   wherein k can be 0, 1, 2, or 3; and wherein m can be 0, 1, 2 or 3.

As noted above, R4 can be a group of formula (II),

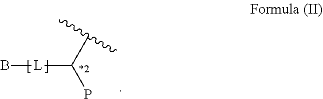

P can be alkoxy, OH or NR11R12. Preferably, P is NR11R12.

-[L]- can be a bond, —[(CH₂)₁₋₄]—, —[(CH₂)—O—(CH₂)]—, or —[O—(CH₂)]. When -[L]- is a bond, B is a C₁₋₄ linear or branched chain hydrocarbon. When -[L]- is —[(CH₂)₁₋₄]—, —[(CH₂)—O—(CH₂)]—, or —[O—(CH₂)]—, B is OH, aryl, heteroaryl, heterocyclyl, cycloalkyl or

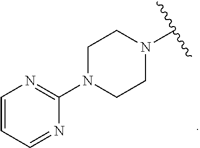 [SMILES: *N1CCN(c2ncccn2)CC1]

As noted above P can be NR11R12. R11 and R12 are as defined above. In particular, when P is NR11R12, R11 and R12 can be independently selected from H, alkyl, and cycloalkyl, or R11 and R12 together with the nitrogen atom to which they are attached form a carbon-containing 5-membered heterocyclic ring. More specifically NR11R12 can be NHR11, where R11 is selected from H, alkyl, and cycloalkyl. Alternatively, NR11R12 is selected from NH₂, NH(iPr) and NH(cyclohexyl). Alternatively NR11R12 is N-linked pyrrolidinyl.

As noted above, when -[L]- is a bond, B is a C₁₋₄ linear or branched chain hydrocarbon.

As noted above, when -[L]- is —[(CH₂)₁₋₄]—, —[(CH₂)—O—(CH₂)]—, or —[O—(CH₂)]—, B is OH, aryl, heteroaryl, heterocyclyl, cycloalkyl or

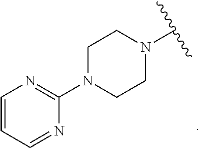 [SMILES: *N1CCN(c2ncccn2)CC1]

Specifically -[L]- can be —[CH₂]—, —[(CH₂)₂]—, —[(CH₂)₃]—, or —[(CH₂)₄]—. More specifically, -[L]- can be —[(CH₂)₂]—, —[(CH₂)₃]—, or —[(CH₂)₄]—. Preferably, -[L]- is selected from —[(CH₂)₂]— and —[(CH₂)₄]—.

When -[L]- is —[(CH₂)₂]—, B can OH, aryl, heteroaryl, heterocyclyl, cycloalkyl or

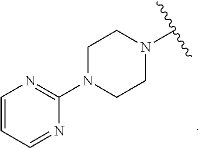 [SMILES: *N1CCN(c2ncccn2)CC1]

Specifically, when -[L]- is —[(CH₂)₂]—, B can be aryl. More specifically, when -[L]- is —[(CH₂)₂]—, B can be phenyl.

When -[L]- is —[(CH₂)₄]—, B can be OH, aryl, heteroaryl, heterocyclyl, cycloalkyl or

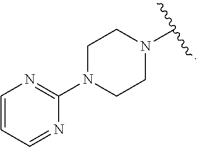 [SMILES: *N1CCN(c2ncccn2)CC1]

Specifically, when -[L]- is —[(CH₂)₄]—, B can be heterocyclyl. More specifically, when -[L]- is —[(CH₂)₄]—, B can be piperidinyl which can be optionally substituted as defined under heterocyclyl, and preferably B is unsubstituted N-linked piperidinyl.

When R4 is a group of formula (II) as outlined above, Y can be a bond or —[CHR5]-. Preferably, Y is a bond. R2^{A} can be a group as defined above. Particularly, R2^{A} can be alkyl, —(CH₂)₀₋₃aryl, —(CH₂)₀₋₃-[benzothiophene], or —(CH₂)₀₋₃-[indole]. More particularly, R2^{A} can be selected from methyl, —(CH₂)-phenyl, —(CH₂)-naphthyl, —(CH₂)-[benzothiophene], —(CH₂)-[indole], and

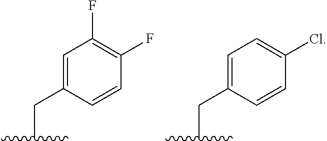 [SMILES: *Cc1ccc(F)c(F)c1 ; *Cc1ccc(Cl)cc1]

Alternatively, when Y is a bond, R1 and R2^{A}, together with the nitrogen atom to which R1 is attached and the carbon atom to which R2^{A} is attached, may be linked by alkylene to form a 4-, 5-, or 6-membered saturated heterocycle, preferably a 4- or 5-membered heterocycle, and preferably a 4-membered heterocycle.

In addition, R3 can be as defined above. In particular, when R4 is a group of formula (II) as above, Y is a bond, and R2^{A} is defined as above, R3 can be a fused 6,5- or 6,6-bicyclic ring, containing one heteroatom selected from S and N, wherein at least one of the rings is aromatic and, optionally the bicyclic ring contains one additional heteroatom independently selected from N, O and S; optionally wherein the fused 6,5- or 6,6-bicyclic ring may be substituted with 1, 2, or 3 substituents selected from alkyl^{b}, alkoxy, OH, NH₂, halo, CN, and CF₃.

In particular, R3 can be a fused 6,5- or 6,6-bicyclic ring, containing one N atom, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃; wherein the fused 6,5-bicyclic ring may be attached via the 6- or 5-membered ring. Particularly, the substituents on the bicyclic ring containing one N atom can be alkyl, preferably methyl; halo, preferably Cl; and NH₂. Preferred R3 groups containing one N atom are:

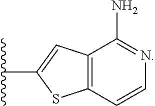 [SMILES: *c1cc2c(N)nccc2s1]

Alternatively, R3 can be a fused 6,5- or 6,6-bicyclic ring, containing two N atoms, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃; wherein the fused 6,5-bicyclic ring may be attached via the 6- or 5-membered ring. Particularly, the substituents on the bicyclic ring containing two N atoms can be alkyl, preferably methyl; halo, preferably Cl; and NH₂. A preferred R3 group containing two N atoms is

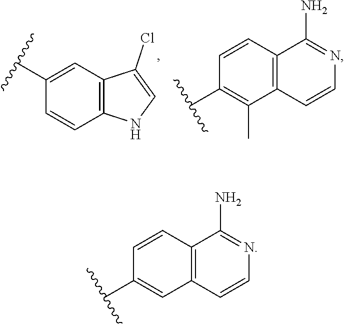

Alternatively, R3 can be a fused 6,5- or 6,6-bicyclic ring, containing one N atom and one S atom, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃; wherein the fused 6,5-bicyclic ring may be attached via the 6- or 5-membered ring. Particularly, the substituents on the bicyclic ring containing one N atom and one S atom can be alkyl, preferably methyl; halo, preferably Cl; and NH₂. A preferred R3 group containing one N atom and one S atom is

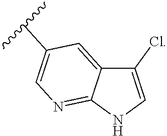 [SMILES: *c1cnc2[nH]cc(Cl)c2c1]

Alternatively, R3 can be a fused 6,5- or 6,6-bicyclic ring, containing one N atom and one O atom, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃; wherein the fused 6,5-bicyclic ring may be attached via the 6- or 5-membered ring. Particularly, the substituents on the bicyclic ring containing one N atom and one O atom can be alkyl, preferably methyl; halo, preferably Cl; and NH₂. A preferred R3 group containing one N atom and one O atom is

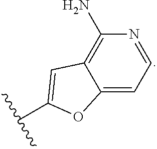 [SMILES: *c1cc2c(N)nccc2o1]

In particular, a preferred group of compounds of formula (I) where R4 is a group of formula (II) as described above, and with the remainder of the compound as described above, are:

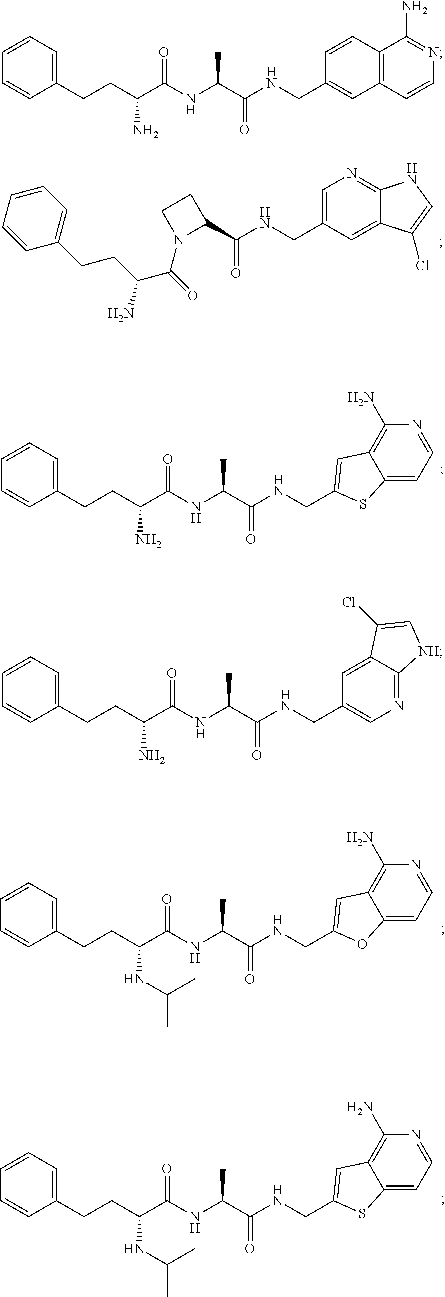

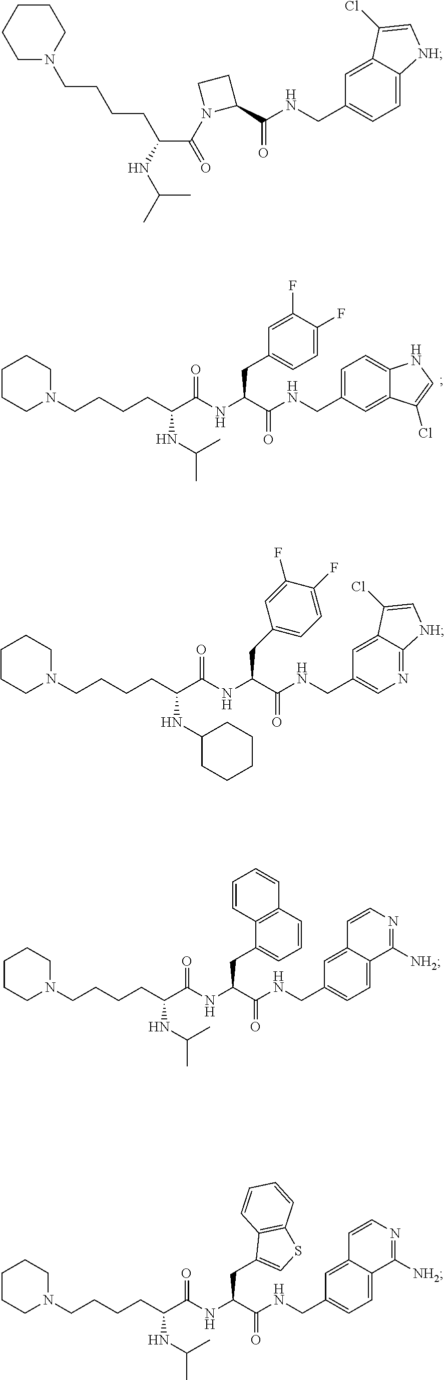 [SMILES: CC(C)N[C@H](CCCCN1CCCCC1)C(=O)N1CC[C@H]1C(=O)NCc1ccc2[nH]cc(Cl)c2c1 ; CC(C)N[C@H](CCCCN1CCCCC1)C(=O)N[C@@H](Cc1ccc(F)c(F)c1)C(=O)NCc1ccc2[nH]cc(Cl)c2c1 ; O=C(NCc1cnc2[nH]cc(Cl)c2c1)[C@H](Cc1ccc(F)c(F)c1)NC(=O)[C@@H](CCCCN1CCCCC1)NC1CCCCC1 ; CC(C)N[C@H](CCCCN1CCCCC1)C(=O)N[C@@H](Cc1cccc2ccccc12)C(=O)NCc1ccc2c(N)nccc2c1 ; CC(C)N[C@H](CCCCN1CCCCC1)C(=O)N[C@@H](Cc1csc2ccccc12)C(=O)NCc1ccc2c(N)nccc2c1]

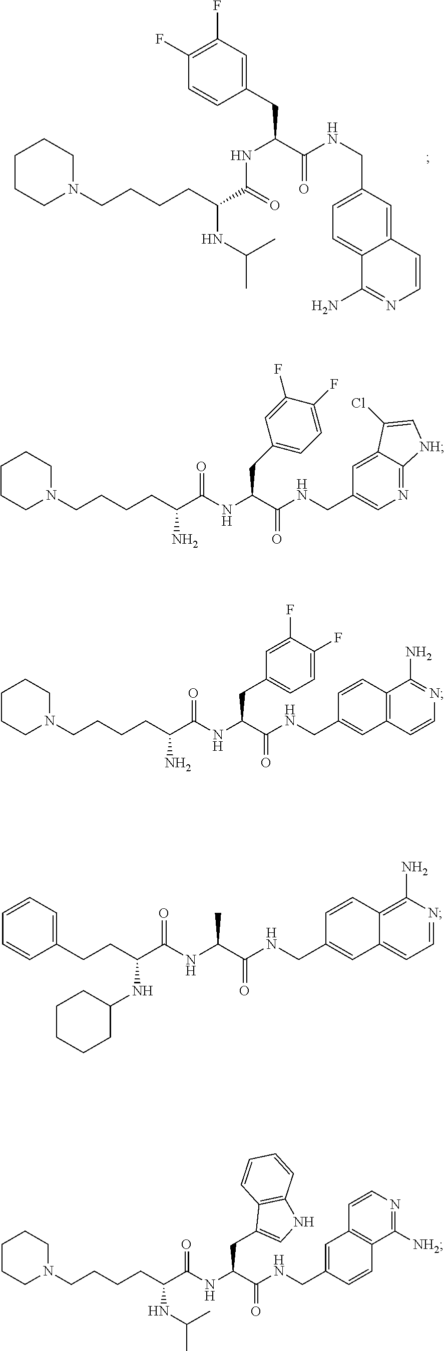 [SMILES: CC(C)N[C@H](CCCCN1CCCCC1)C(=O)N[C@@H](Cc1ccc(F)c(F)c1)C(=O)NCc1ccc2c(N)nccc2c1 ; N[C@H](CCCCN1CCCCC1)C(=O)N[C@@H](Cc1ccc(F)c(F)c1)C(=O)NCc1cnc2[nH]cc(Cl)c2c1 ; Nc1nccc2cc(CNC(=O)[C@H](Cc3ccc(F)c(F)c3)NC(=O)[C@H](N)CCCCN3CCCCC3)ccc12 ; C[C@H](NC(=O)[C@@H](CCc1ccccc1)NC1CCCCC1)C(=O)NCc1ccc2c(N)nccc2c1 ; CC(C)N[C@H](CCCCN1CCCCC1)C(=O)N[C@@H](Cc1c[nH]c2ccccc12)C(=O)NCc1ccc2c(N)nccc2c1]

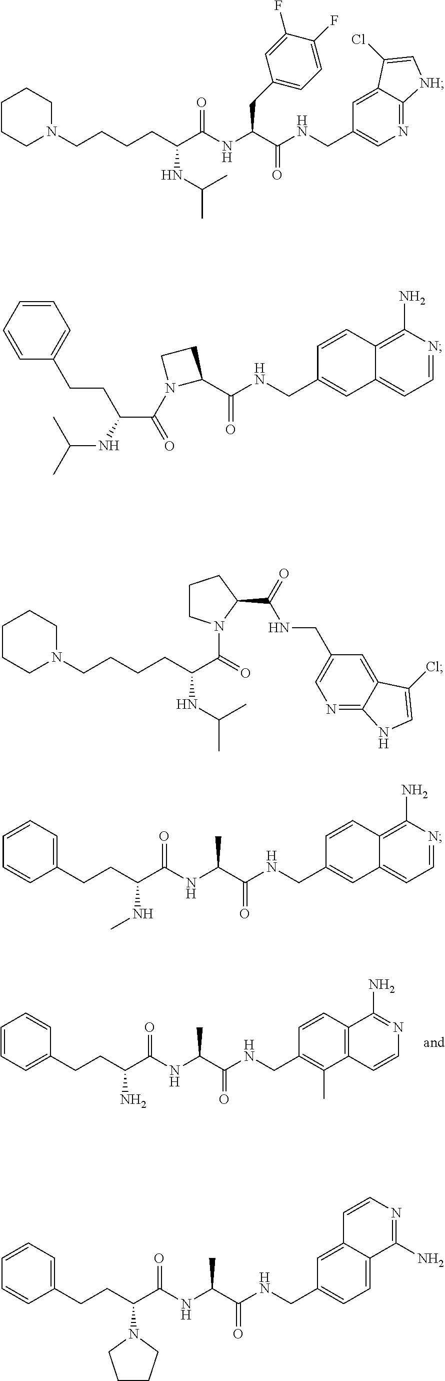 [SMILES: CC(C)N[C@H](CCCCN1CCCCC1)C(=O)N[C@@H](Cc1ccc(F)c(F)c1)C(=O)NCc1cnc2[nH]cc(Cl)c2c1 ; CC(C)N[C@H](CCc1ccccc1)C(=O)N1CC[C@H]1C(=O)NCc1ccc2c(N)nccc2c1 ; CC(C)N[C@H](CCCCN1CCCCC1)C(=O)N1CCC[C@H]1C(=O)NCc1cnc2[nH]cc(Cl)c2c1 ; CN[C@H](CCc1ccccc1)C(=O)N[C@@H](C)C(=O)NCc1ccc2c(N)nccc2c1 ; Cc1c(CNC(=O)[C@H](C)NC(=O)[C@H](N)CCc2ccccc2)ccc2c(N)nccc12 ; C[C@H](NC(=O)[C@@H](CCc1ccccc1)N1CCCC1)C(=O)NCc1ccc2c(N)nccc2c1]

and pharmaceutically acceptable salts and/or solvates thereof.

In particular, an even more preferred group of compounds of formula (I) where R4 is a group of formula (II) as described above, and with the remainder of the compound as described above, are:

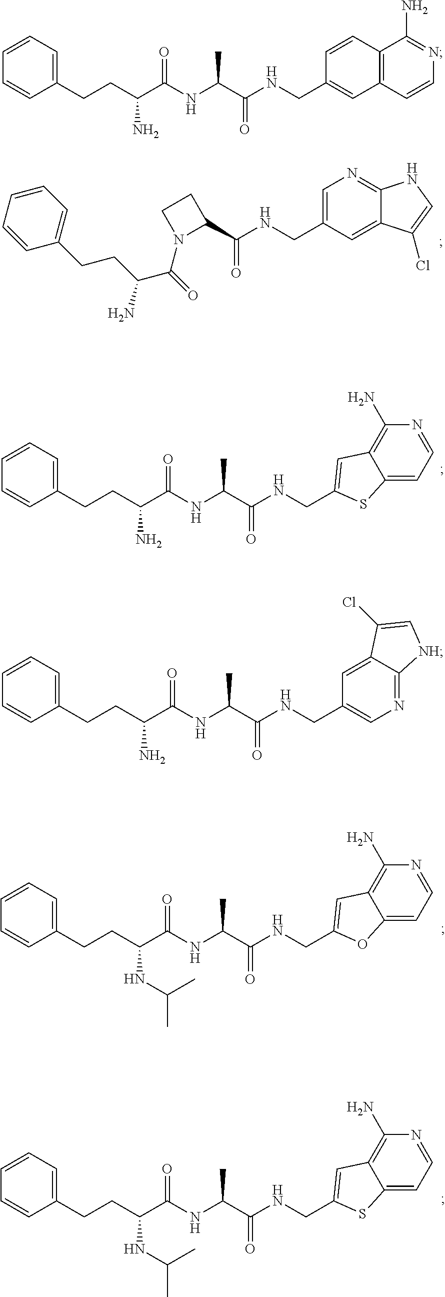

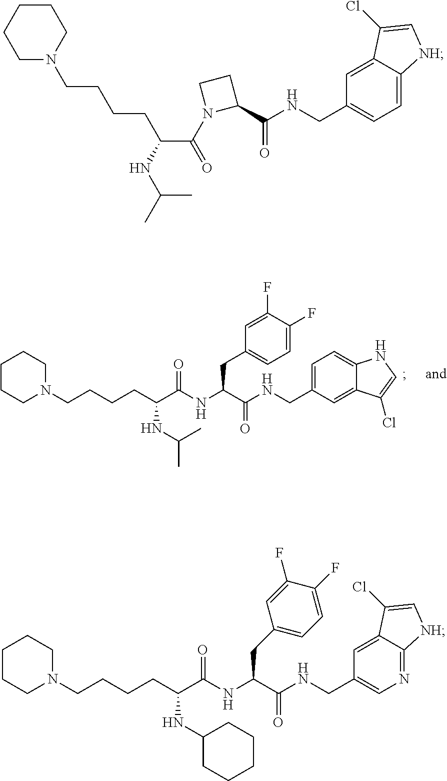 [SMILES: CC(C)N[C@H](CCCCN1CCCCC1)C(=O)N1CC[C@H]1C(=O)NCc1ccc2[nH]cc(Cl)c2c1 ; CC(C)N[C@H](CCCCN1CCCCC1)C(=O)N[C@@H](Cc1ccc(F)c(F)c1)C(=O)NCc1ccc2[nH]cc(Cl)c2c1 ; O=C(NCc1cnc2[nH]cc(Cl)c2c1)[C@H](Cc1ccc(F)c(F)c1)NC(=O)[C@@H](CCCCN1CCCCC1)NC1CCCCC1]

and; and pharmaceutically acceptable salts and/or solvates thereof.

Alternatively, as noted above, R4 can be —(CH₂)ₘ-(heterocyclyl). m can be 0, 1, 2 or 3. More particularly, m can be 0.

When m is 0, R4 can be heterocyclyl. In particular, when m is 0, R4 can be a 5- or 6-membered carbon-containing non-aromatic ring containing one or two ring members selected from N and O, which can be substituted as defined under heterocyclyl above. Specifically, when m is 0, R4 can be a 5-membered carbon-containing non-aromatic ring containing one N ring member, such as pyrrolidinyl. When m is 0 and R4 is a 5-membered carbon-containing non-aromatic ring containing one N ring member as above, Y can be a bond and R2^{A} can be —(CH₂)₀₋₃aryl, more specifically —(CH₂)-aryl, and more specifically —(CH₂)-naphthyl. Here, R3 can be phenyl, which may be optionally substituted with 1, 2 or 3 substituents independently selected from alkyl^{b}, alkoxy, OH, NH₂ halo, CN, CF₃, —C(═NH)NH₂, and heteroaryl^{b}. More specifically, R3 can be phenyl, optionally substituted with —C(═NH)NH₂, for example

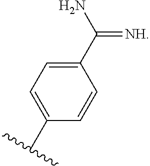 [SMILES: *c1ccc(C(=N)N)cc1]

Alternatively, when m is 0, R4 can be a 6-membered carbon containing non-aromatic ring containing one N ring member such as piperidinyl, optionally substituted with alkyl, such as isopropyl, for example

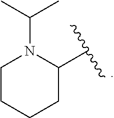 [SMILES: *C1CCCCN1C(C)C]

When m is 0 and R4 is a 6-membered carbon-containing non-aromatic ring containing one N ring member as above, Y can be a bond and R2^{A} can be —(CH₂)₀₋₃aryl, more specifically —(CH₂)-aryl, and more specifically

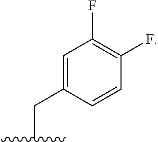 [SMILES: *Cc1ccc(F)c(F)c1]

Here, R3 can be a fused 6,6-bicyclic ring, containing one N atom, wherein at least one of the rings is aromatic, optionally wherein the fused 6,6-bicyclic ring can be substituted with 1, 2, or 3 substituents selected from alkyl^{b}, alkoxy, OH, NH₂, halo, CN, and CF₃. In particular here, R3 can be an unsubstituted 6,6-bicyclic ring, containing one N atom, for example

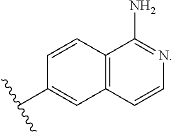 [SMILES: *c1ccc2c(N)nccc2c1]

As a further alternative, R4 can be a heterocyclyl group, as defined above, where two ring atoms on heterocyclyl are linked with an heteroalkylene to form a non-aromatic ring containing 5, 6, or 7 ring members, and more specifically the two ring atoms on heterocyclyl can be linked by an heteroalkylene to form a non-aromatic ring containing 5 ring members, for example

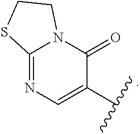 [SMILES: *c1cnc2n(c1=O)CCS2]

When m is 0 and R4 is the heterocyclyl group with two ring atoms on heterocyclyl linked by an heteroalkylene to form a non-aromatic ring containing 5 ring members, as outlined above, Y can be a bond and R2^{A} can be —(CH₂)₀₋₃aryl, more specifically —(CH₂)-aryl, and more specifically

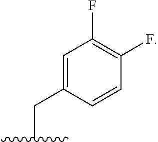 [SMILES: *Cc1ccc(F)c(F)c1]

Here, R3 can be a fused 6,6-bicyclic ring, containing one N atom, wherein at least one of the rings is aromatic, optionally wherein the fused 6,6-bicyclic ring can be substituted with 1, 2, or 3 substituents selected from alkyl^{b}, alkoxy, OH, NH₂, halo, CN, and CF₃. In particular here, R3 can be an unsubstituted 6,6-bicyclic ring, containing one N atom, for example

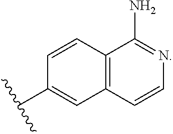 [SMILES: *c1ccc2c(N)nccc2c1]

In particular, a preferred group of compounds of formula (I) where R4 is —(CH₂)ₘ-(heterocyclyl) as described above, and with the remainder of the compound as described above, are:

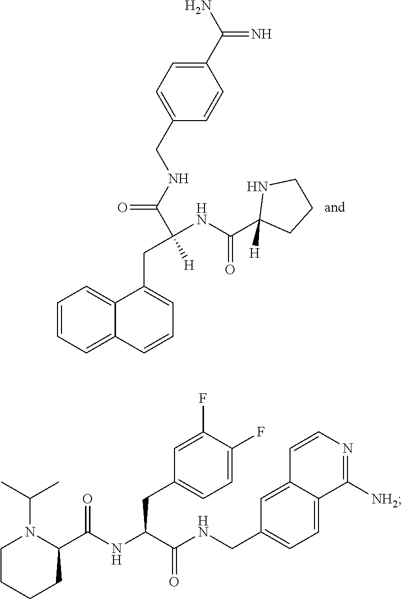 [SMILES: N=C(N)c1ccc(CNC(=O)[C@H](Cc2cccc3ccccc23)NC(=O)[C@H]2CCCN2)cc1 ; CC(C)N1CCCC[C@@H]1C(=O)N[C@@H](Cc1ccc(F)c(F)c1)C(=O)NCc1ccc2c(N)nccc2c1]

and pharmaceutically acceptable salts and/or solvates thereof.

Alternatively, as noted above, R4 can be —(CH₂)ₘ—(NR8R9). m can be 0, 1, 2 or 3. More particularly, m can be 1.

When m is 1, R4 can be —(CH₂)—(NR8R9). R8 and R9 can be as defined above. In particular, R8 and R9 together with the nitrogen atom to which they are attached can form a carbon-containing 4-, 5-, 6- or 7-membered heterocyclic ring, optionally containing an additional heteroatom selected from N, NR10, S, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃. Specifically, R8 and R9 together with the nitrogen atom to which they are attached can form a carbon-containing 4-, 5-, 6- or 7-membered heterocyclic ring, which may be optionally mono- or di-substituted with substituents independently selected from oxo, alkyl^{b}, alkoxy, OH, halo, —SO₂CH₃, and CF₃. More specifically, R8 and R9 together with the nitrogen atom to which they are attached can form a carbon-containing 6-membered heterocyclic ring, which may be optionally mono- or di-substituted with substituents independently selected from oxo, alkyl^{b}, alkoxy, OH, halo, —SO₂CH₃, and CF₃, and preferably the 6-membered heterocyclic ring is di-substituted with alkyl^{b} substituents, and preferably the alkyl^{b} substituents are alkyl, for example

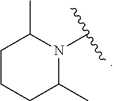 [SMILES: *N1C(C)CCCC1C]

When m is 1, and R4 is defined as —(CH₂)—(NR8R9) as above, Y can be a bond, or —[CHR5]-. Preferably, Y is a bond. R2^{A} can be a group as defined above. In particular, R2^{A} can be —(CH₂)₀₋₃aryl, —(CH₂)₀₋₃-[benzothiophene] or —(CH₂)₀₋₃-[indole]. More particularly, R2^{A} can be —(CH₂)-[benzothiophene], —(CH₂)-[indole], —(CH₂)-naphthyl-(CH₂)-phenyl,

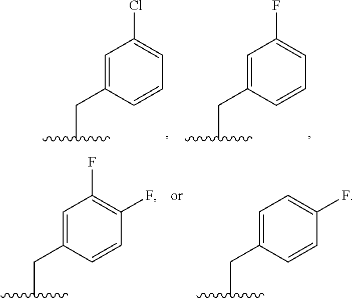

In addition, R3 can be defined as above. In particular, when R4 is —(CH₂)—(NR8R9) as defined above, Y is a bond, and R2^{A} is defined as above, R3 can be a fused 6,6-bicyclic ring, containing one N atom, wherein at least one of the rings is aromatic, optionally wherein the fused 6,6-bicyclic ring may be substituted with 1, 2, or 3 substituents selected from alkyl^{b}, alkoxy, OH, NH₂, halo, CN, and CF₃. In particular here, R3 can be an unsubstituted 6,6-bicyclic ring, containing one N atom, for example

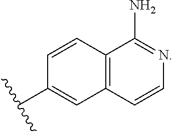 [SMILES: *c1ccc2c(N)nccc2c1]

In particular, a preferred group of compounds of formula (I) where R4 is —(CH₂)ₘ—(NR8R9) as described above, and with the remainder of the compound as described above, are:

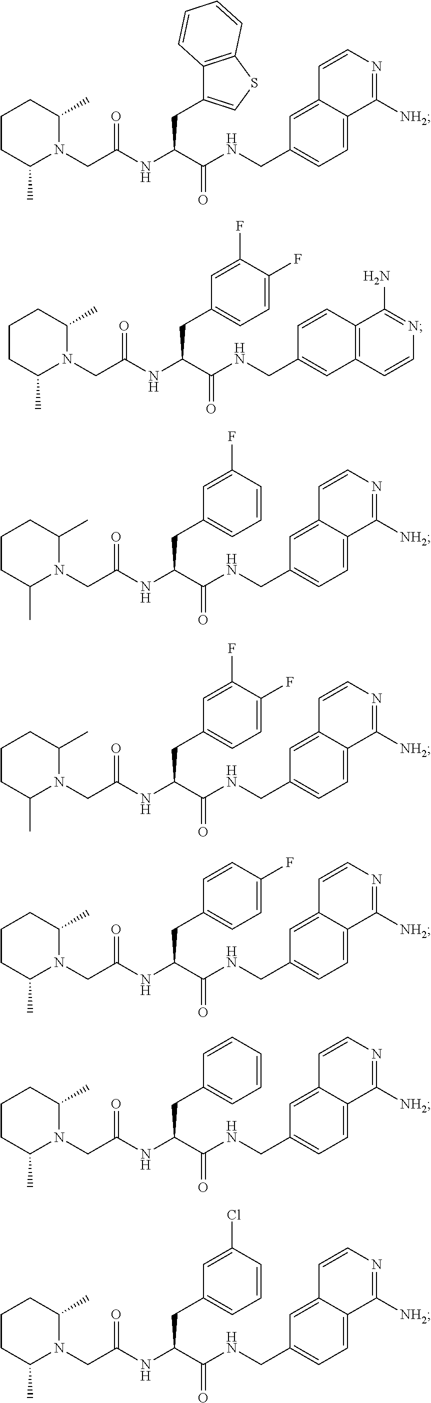

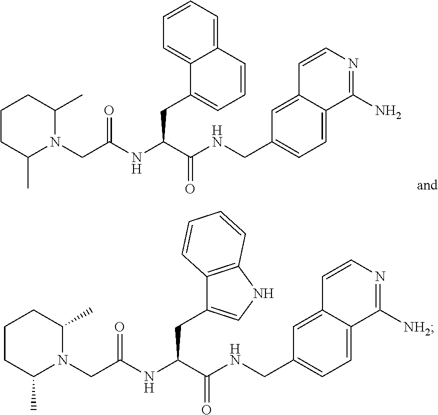

and pharmaceutically acceptable salts and/or solvates thereof.

Alternatively, R4 can be:

—(CH₂)ₘ-[fused 6,5- or 6,6-heteroaromatic bicyclic ring], wherein at least one ring atom is a heteroatom selected from O, N or S, and optionally, 1, 2 or 3 additional ring atoms can be selected from N or N H; wherein the fused 6,5- or 6,6-heteroaromatic bicyclic ring can be optionally substituted with 1, 2 or 3 substituents independently selected from alkyl^{b}; wherein the 6,5-heteroaromatic bicyclic ring can be attached to —(CH₂)ₘ— via the 6- or 5-membered ring; or,
R4 can be methyl, —C(CH₃)₂(OH), —C(CH₃)₂(NHMe), —(CH₂)ₘ-(aryl), —(CH₂)ₘ-(cycloalkyl), —(CH₂)ₘ-(heteroaryl), —(CH₂)-(alkyl), —(CH(halo)₂), —(CH₂)ₘ—(NR10R7), —(CH₂)ₘ—O—(CH₂)ₖ-(aryl), —(CH₂)ₘ—(SO₂)—(CH₂)ₖ-(aryl), —(CH₂)ₘ-(alkoxy), —(CH₂)ₘ—O—(CH₂)ₖ-(heteroaryl), or —(CH₂)ₘ-[pyridone, which may be optionally substituted by alkyl^{b}, or CF₃]. m can be 0, 1, 2 or 3 and k can be 0, 1, 2, or 3.

R4 can be —(CH₂)ₘ-(heteroaryl), and in particular m can be 0, 1 or 2. More specifically, m can be 0. The heteroaryl group in —(CH₂)ₘ-(heteroaryl) is as defined above. When m=0, R4 can be

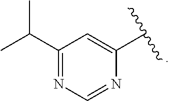 [SMILES: *c1cc(C(C)C)ncn1]

Y can be a bond and R2^{A} can be —(CH₂)₀₋₃aryl, more specifically —(CH₂)-aryl, and more specifically

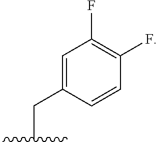 [SMILES: *Cc1ccc(F)c(F)c1]

Here, R3 can be a fused 6,6-bicyclic ring, containing one N atom, wherein at least one of the rings is aromatic, optionally wherein the fused 6,6-bicyclic ring can be substituted with 1, 2, or 3 substituents selected from alkyl^{b}, alkoxy, OH, NH₂, halo, CN, and CF₃. In particular here, R3 can be an unsubstituted 6,6-bicyclic ring, containing one N atom, for example

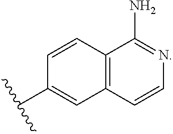 [SMILES: *c1ccc2c(N)nccc2c1]

A can also be —SO₂R6. When A is —SO₂R6, preferably R3 is

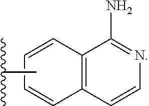

When A is SO₂R6, Y can be a bond. When A is —SO₂R6, R2^{A} can be alkyl and —(CH₂)₀₋₃aryl. When A is —SO₂R6, R6 can be —(CH₂)ₘ-(aryl), —(CH₂)-(alkyl). In particular here, R3 can be a fused 6,6-bicyclic ring, containing one N atom, wherein at least one of the rings is aromatic, optionally wherein the fused 6,6-bicyclic ring may be substituted with 1, 2, or 3 substituents selected from alkyl^{b}, alkoxy, OH, NH₂, halo, CN, and CF₃. In particular here, R3 can be an unsubstituted 6,6-bicyclic ring, containing one N atom, for example

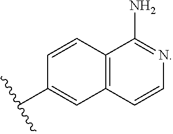 [SMILES: *c1ccc2c(N)nccc2c1]

n can also be 2.

When n is 2, R3 can be

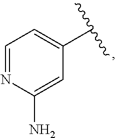 [SMILES: *c1ccnc(N)c1]

R2^{A} can be alkyl, preferably methyl, Y is a bond, and A can be —(C═O)R4, where R4 is a group of formula (II), B is preferably phenyl, -[L]- is preferably —[(CH₂)₂]—, and P is NH(iPr).

As noted above, *1 denotes a chiral centre. Preferably, chiral centre *1 is in the (S)-configuration.

As noted above, *2 denotes a chiral centre. Preferably, chiral centre *2 is in the (R)-configuration.

A preferred group of compounds of formula (I) is:

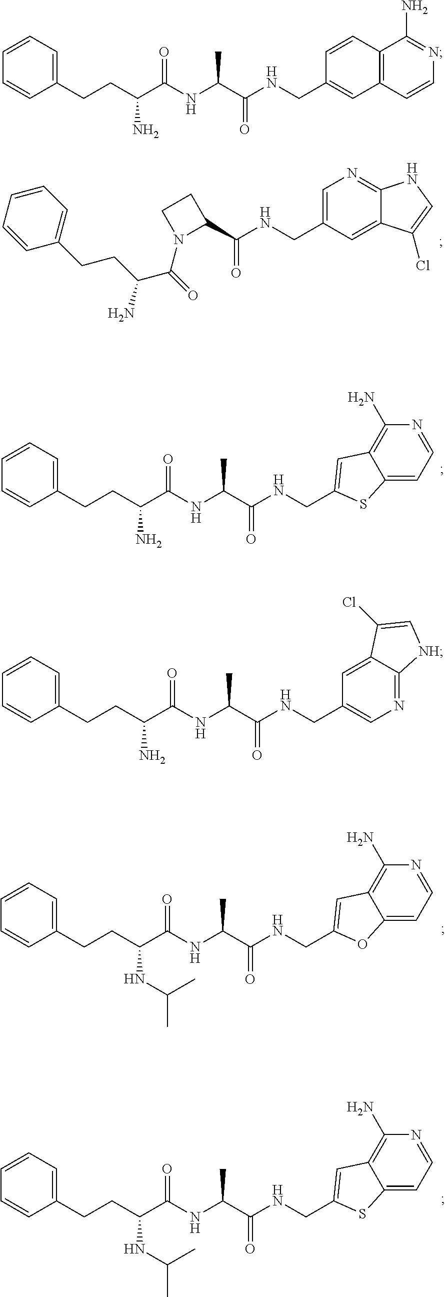

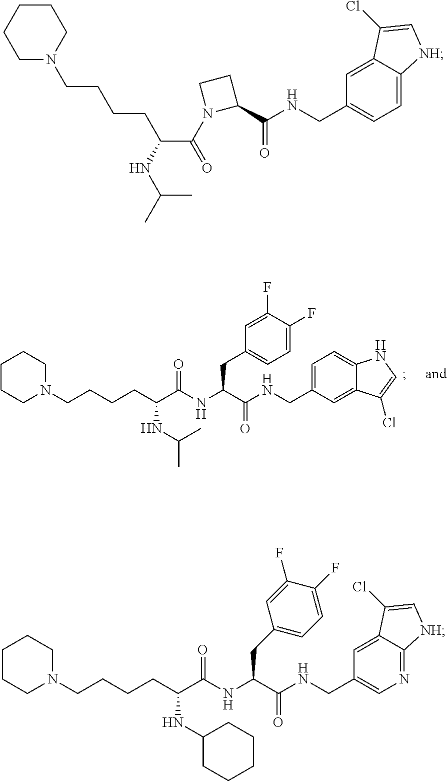 [SMILES: CC(C)N[C@H](CCCCN1CCCCC1)C(=O)N1CC[C@H]1C(=O)NCc1ccc2[nH]cc(Cl)c2c1 ; CC(C)N[C@H](CCCCN1CCCCC1)C(=O)N[C@@H](Cc1ccc(F)c(F)c1)C(=O)NCc1ccc2[nH]cc(Cl)c2c1 ; O=C(NCc1cnc2[nH]cc(Cl)c2c1)[C@H](Cc1ccc(F)c(F)c1)NC(=O)[C@@H](CCCCN1CCCCC1)NC1CCCCC1]

and pharmaceutically acceptable salts and/or solvates thereof.

An alternative group of preferred compounds of formula (I) is:

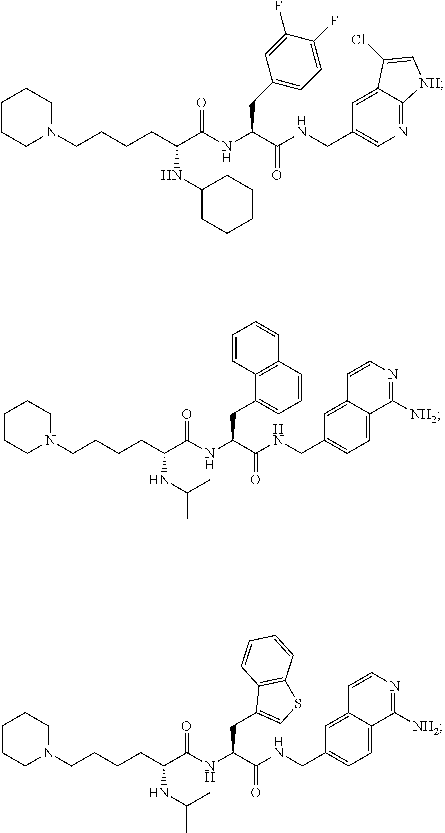 [SMILES: O=C(NCc1cnc2[nH]cc(Cl)c2c1)[C@H](Cc1ccc(F)c(F)c1)NC(=O)[C@@H](CCCCN1CCCCC1)NC1CCCCC1 ; CC(C)N[C@H](CCCCN1CCCCC1)C(=O)N[C@@H](Cc1cccc2ccccc12)C(=O)NCc1ccc2c(N)nccc2c1 ; CC(C)N[C@H](CCCCN1CCCCC1)C(=O)N[C@@H](Cc1csc2ccccc12)C(=O)NCc1ccc2c(N)nccc2c1]

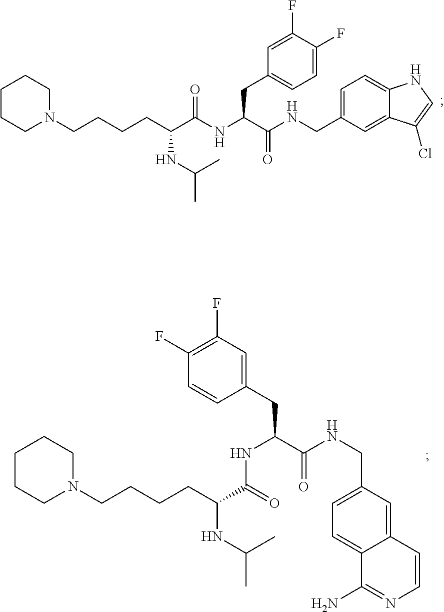 [SMILES: CC(C)N[C@H](CCCCN1CCCCC1)C(=O)N[C@@H](Cc1ccc(F)c(F)c1)C(=O)NCc1ccc2[nH]cc(Cl)c2c1 ; CC(C)N[C@H](CCCCN1CCCCC1)C(=O)N[C@@H](Cc1ccc(F)c(F)c1)C(=O)NCc1ccc2c(N)nccc2c1]

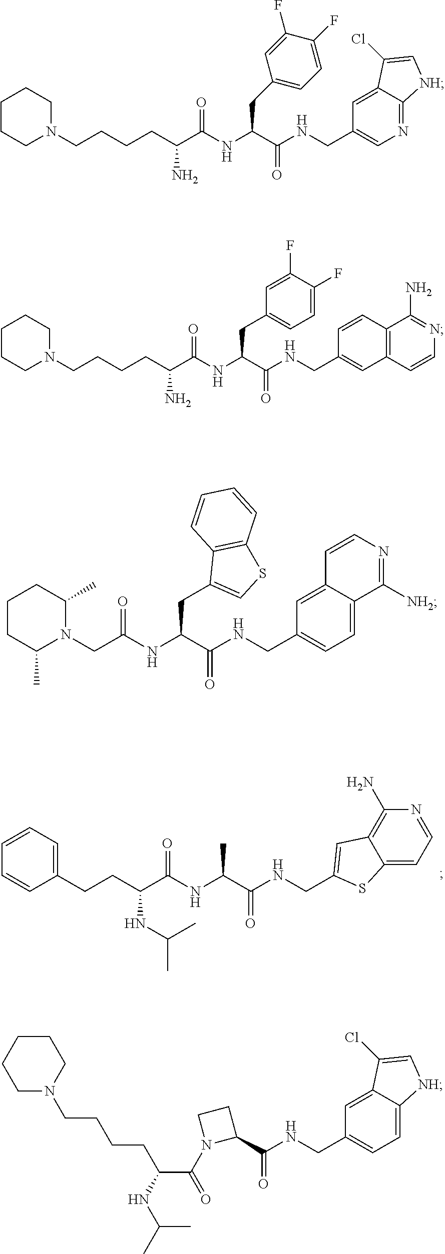 [SMILES: N[C@H](CCCCN1CCCCC1)C(=O)N[C@@H](Cc1ccc(F)c(F)c1)C(=O)NCc1cnc2[nH]cc(Cl)c2c1 ; Nc1nccc2cc(CNC(=O)[C@H](Cc3ccc(F)c(F)c3)NC(=O)[C@H](N)CCCCN3CCCCC3)ccc12 ; C[C@@H]1CCC[C@H](C)N1CC(=O)N[C@@H](Cc1csc2ccccc12)C(=O)NCc1ccc2c(N)nccc2c1 ; CC(C)N[C@H](CCc1ccccc1)C(=O)N[C@@H](C)C(=O)NCc1cc2c(N)nccc2s1 ; CC(C)N[C@H](CCCCN1CCCCC1)C(=O)N1CC[C@H]1C(=O)NCc1ccc2[nH]cc(Cl)c2c1]

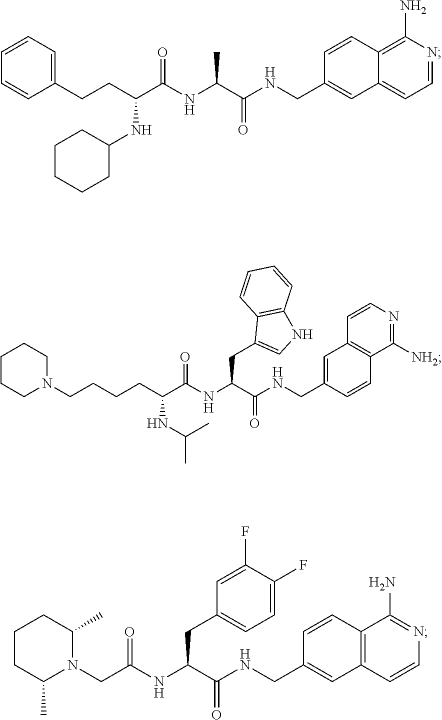 [SMILES: C[C@H](NC(=O)[C@@H](CCc1ccccc1)NC1CCCCC1)C(=O)NCc1ccc2c(N)nccc2c1 ; CC(C)N[C@H](CCCCN1CCCCC1)C(=O)N[C@@H](Cc1c[nH]c2ccccc12)C(=O)NCc1ccc2c(N)nccc2c1 ; C[C@@H]1CCC[C@H](C)N1CC(=O)N[C@@H](Cc1ccc(F)c(F)c1)C(=O)NCc1ccc2c(N)nccc2c1]

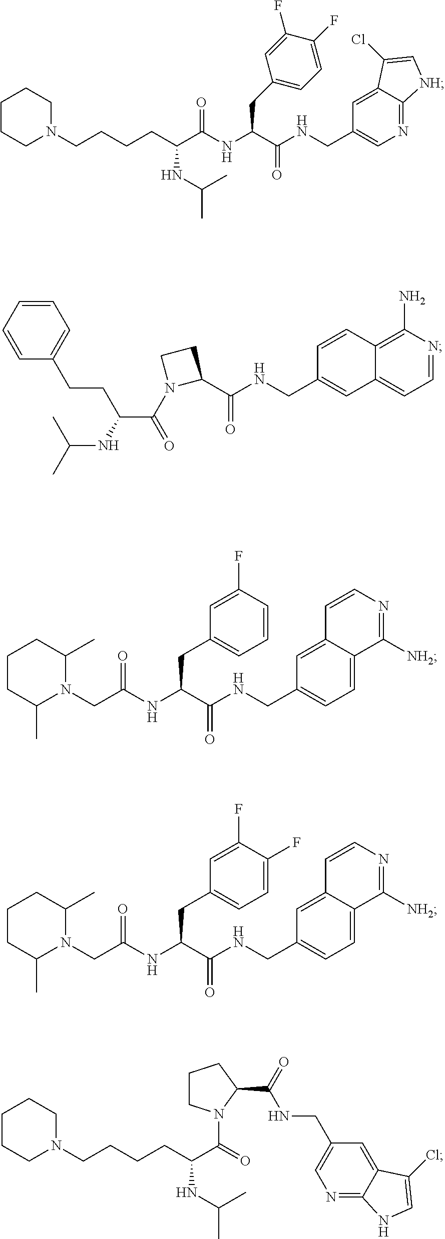 [SMILES: CC(C)N[C@H](CCCCN1CCCCC1)C(=O)N[C@@H](Cc1ccc(F)c(F)c1)C(=O)NCc1cnc2[nH]cc(Cl)c2c1 ; CC(C)N[C@H](CCc1ccccc1)C(=O)N1CC[C@H]1C(=O)NCc1ccc2c(N)nccc2c1 ; CC1CCCC(C)N1CC(=O)N[C@@H](Cc1cccc(F)c1)C(=O)NCc1ccc2c(N)nccc2c1 ; CC1CCCC(C)N1CC(=O)N[C@@H](Cc1ccc(F)c(F)c1)C(=O)NCc1ccc2c(N)nccc2c1 ; CC(C)N[C@H](CCCCN1CCCCC1)C(=O)N1CCC[C@H]1C(=O)NCc1cnc2[nH]cc(Cl)c2c1]

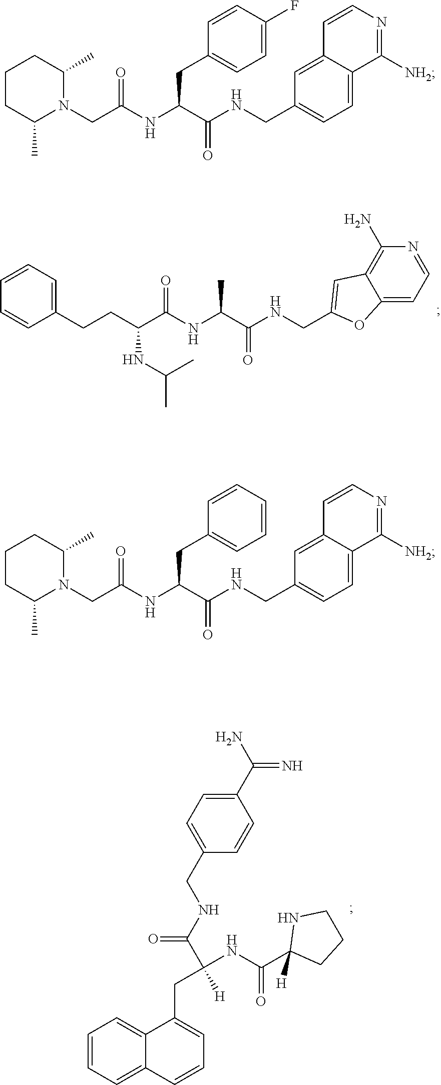 [SMILES: C[C@@H]1CCC[C@H](C)N1CC(=O)N[C@@H](Cc1ccc(F)cc1)C(=O)NCc1ccc2c(N)nccc2c1 ; CC(C)N[C@H](CCc1ccccc1)C(=O)N[C@@H](C)C(=O)NCc1cc2c(N)nccc2o1 ; C[C@@H]1CCC[C@H](C)N1CC(=O)N[C@@H](Cc1ccccc1)C(=O)NCc1ccc2c(N)nccc2c1 ; N=C(N)c1ccc(CNC(=O)[C@H](Cc2cccc3ccccc23)NC(=O)[C@H]2CCCN2)cc1]

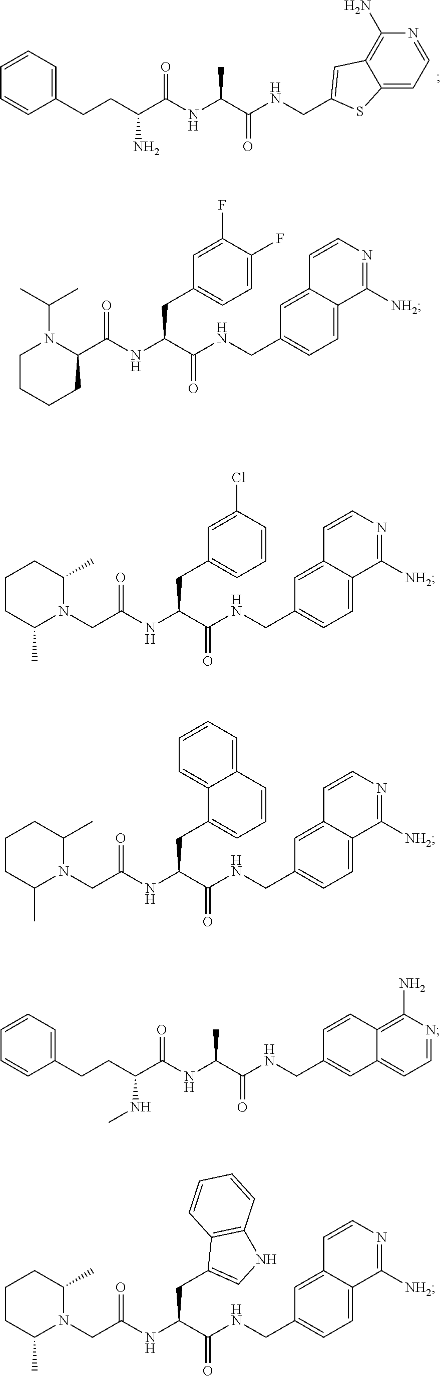 [SMILES: C[C@H](NC(=O)[C@H](N)CCc1ccccc1)C(=O)NCc1cc2c(N)nccc2s1 ; CC(C)N1CCCC[C@@H]1C(=O)N[C@@H](Cc1ccc(F)c(F)c1)C(=O)NCc1ccc2c(N)nccc2c1 ; C[C@@H]1CCC[C@H](C)N1CC(=O)N[C@@H](Cc1cccc(Cl)c1)C(=O)NCc1ccc2c(N)nccc2c1 ; CC1CCCC(C)N1CC(=O)N[C@@H](Cc1cccc2ccccc12)C(=O)NCc1ccc2c(N)nccc2c1 ; CN[C@H](CCc1ccccc1)C(=O)N[C@@H](C)C(=O)NCc1ccc2c(N)nccc2c1 ; C[C@@H]1CCC[C@H](C)N1CC(=O)N[C@@H](Cc1c[nH]c2ccccc12)C(=O)NCc1ccc2c(N)nccc2c1]

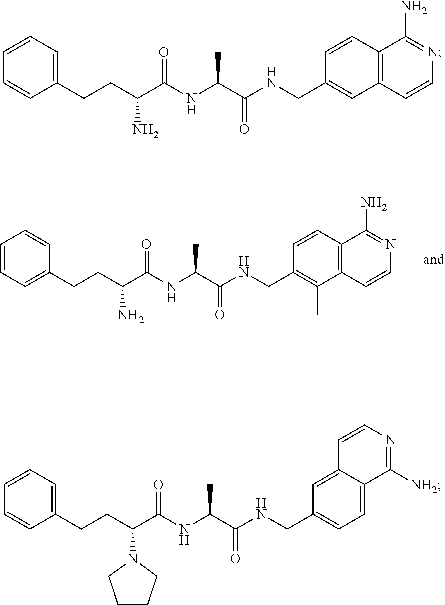 [SMILES: C[C@H](NC(=O)[C@H](N)CCc1ccccc1)C(=O)NCc1ccc2c(N)nccc2c1 ; Cc1c(CNC(=O)[C@H](C)NC(=O)[C@H](N)CCc2ccccc2)ccc2c(N)nccc12 ; C[C@H](NC(=O)[C@@H](CCc1ccccc1)N1CCCC1)C(=O)NCc1ccc2c(N)nccc2c1]

and pharmaceutically acceptable salts and/or solvates thereof.

An even more preferred group of compounds of formula (I) is:

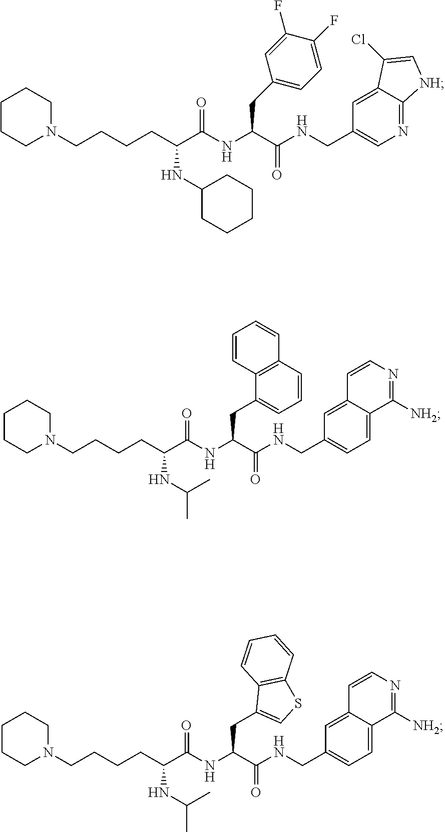 [SMILES: O=C(NCc1cnc2[nH]cc(Cl)c2c1)[C@H](Cc1ccc(F)c(F)c1)NC(=O)[C@@H](CCCCN1CCCCC1)NC1CCCCC1 ; CC(C)N[C@H](CCCCN1CCCCC1)C(=O)N[C@@H](Cc1cccc2ccccc12)C(=O)NCc1ccc2c(N)nccc2c1 ; CC(C)N[C@H](CCCCN1CCCCC1)C(=O)N[C@@H](Cc1csc2ccccc12)C(=O)NCc1ccc2c(N)nccc2c1]

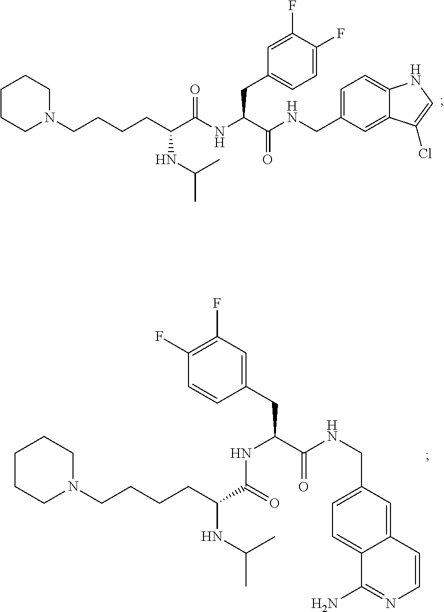 [SMILES: CC(C)N[C@H](CCCCN1CCCCC1)C(=O)N[C@@H](Cc1ccc(F)c(F)c1)C(=O)NCc1ccc2[nH]cc(Cl)c2c1 ; CC(C)N[C@H](CCCCN1CCCCC1)C(=O)N[C@@H](Cc1ccc(F)c(F)c1)C(=O)NCc1ccc2c(N)nccc2c1]

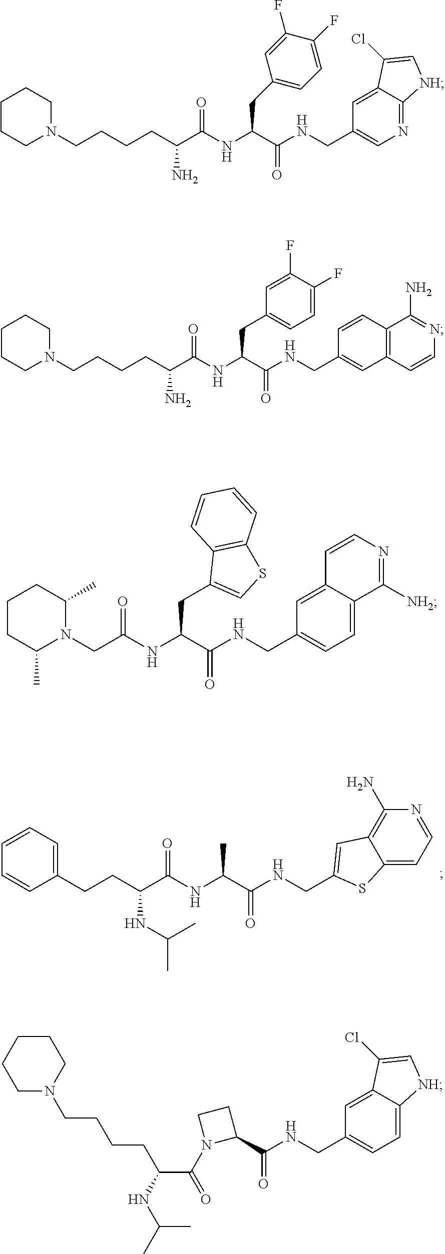 [SMILES: N[C@H](CCCCN1CCCCC1)C(=O)N[C@@H](Cc1ccc(F)c(F)c1)C(=O)NCc1cnc2[nH]cc(Cl)c2c1 ; Nc1nccc2cc(CNC(=O)[C@H](Cc3ccc(F)c(F)c3)NC(=O)[C@H](N)CCCCN3CCCCC3)ccc12 ; C[C@@H]1CCC[C@H](C)N1CC(=O)N[C@@H](Cc1csc2ccccc12)C(=O)NCc1ccc2c(N)nccc2c1 ; CC(C)N[C@H](CCc1ccccc1)C(=O)N[C@@H](C)C(=O)NCc1cc2c(N)nccc2s1 ; CC(C)N[C@H](CCCCN1CCCCC1)C(=O)N1CC[C@H]1C(=O)NCc1ccc2[nH]cc(Cl)c2c1]

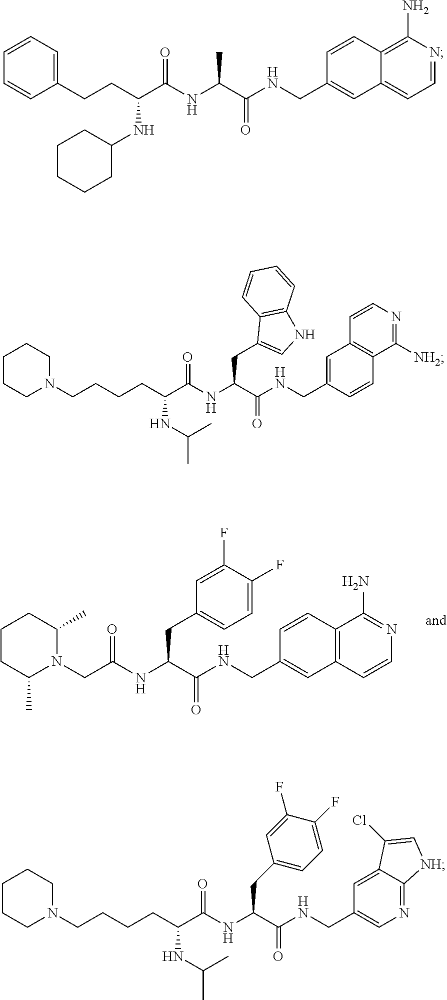 [SMILES: C[C@H](NC(=O)[C@@H](CCc1ccccc1)NC1CCCCC1)C(=O)NCc1ccc2c(N)nccc2c1 ; CC(C)N[C@H](CCCCN1CCCCC1)C(=O)N[C@@H](Cc1c[nH]c2ccccc12)C(=O)NCc1ccc2c(N)nccc2c1 ; C[C@@H]1CCC[C@H](C)N1CC(=O)N[C@@H](Cc1ccc(F)c(F)c1)C(=O)NCc1ccc2c(N)nccc2c1 ; CC(C)N[C@H](CCCCN1CCCCC1)C(=O)N[C@@H](Cc1ccc(F)c(F)c1)C(=O)NCc1cnc2[nH]cc(Cl)c2c1]

and pharmaceutically acceptable salts and/or solvates thereof.

A yet more preferred group of compounds of formula (I) is:

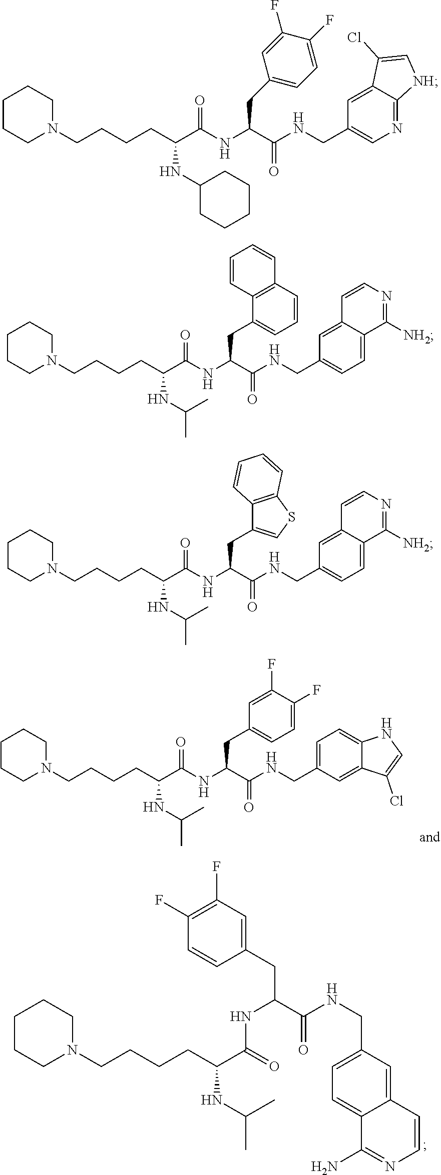

and pharmaceutically acceptable salts and/or solvates thereof.

The present invention also encompasses, but is not limited to, the compounds below in Tables 1 to 23, and pharmaceutically acceptable salts and/or solvates thereof.

The compounds of the invention can be selected from Table 1, and pharmaceutically acceptable salts and/or solvates thereof.

The compounds of the invention can be selected from Table 2, and pharmaceutically acceptable salts and/or solvates thereof.

The compounds of the invention can be selected from Table 3, and pharmaceutically acceptable salts and/or solvates thereof.

The compounds of the invention can be selected from Table 4, and pharmaceutically acceptable salts and/or solvates thereof.

The compounds of the invention can be selected from Table 5, and pharmaceutically acceptable salts and/or solvates thereof.

The compounds of the invention can be selected from Table 6, and pharmaceutically acceptable salts and/or solvates thereof.

The compounds of the invention can be selected from Table 7, and pharmaceutically acceptable salts and/or solvates thereof.

The compounds of the invention can be selected from Table 8, and pharmaceutically acceptable salts and/or solvates thereof.

The compounds of the invention can be selected from Table 9, and pharmaceutically acceptable salts and/or solvates thereof.

The compounds of the invention can be selected from Table 10, and pharmaceutically acceptable salts and/or solvates thereof.

The compounds of the invention can be selected from Table 11, and pharmaceutically acceptable salts and/or solvates thereof.

The compounds of the invention can be selected from Table 12, and pharmaceutically acceptable salts and/or solvates thereof.

The compounds of the invention can be selected from Table 13, and pharmaceutically acceptable salts and/or solvates thereof.

The compounds of the invention can be selected from Table 14, and pharmaceutically acceptable salts and/or solvates thereof.

The compounds of the invention can be selected from Table 15, and pharmaceutically acceptable salts and/or solvates thereof.

The compounds of the invention can be selected from Table 16, and pharmaceutically acceptable salts and/or solvates thereof.

The compounds of the invention can be selected from Table 17, and pharmaceutically acceptable salts and/or solvates thereof.

The compounds of the invention can be selected from Table 18, and pharmaceutically acceptable salts and/or solvates thereof.

The compounds of the invention can be selected from Table 19, and pharmaceutically acceptable salts and/or solvates thereof.

The compounds of the invention can be selected from Table 20, and pharmaceutically acceptable salts and/or solvates thereof.

The compounds of the invention can be selected from Table 21, and pharmaceutically acceptable salts and/or solvates thereof.

The compounds of the invention can be selected from Table 22, and pharmaceutically acceptable salts and/or solvates thereof.

The compounds of the invention can be selected from Table 23, and pharmaceutically acceptable salts and/or solvates thereof.

TABLE 1

| Structure | Example |
|---|---|
| Molecular formula | No. |
| 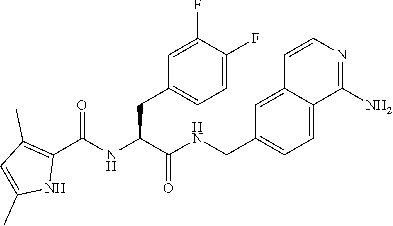 [SMILES: Cc1cc(C)c(C(=O)N[C@@H](Cc2ccc(F)c(F)c2)C(=O)NCc2ccc3c(N)nccc3c2)[nH]1] | 0.01 |
| C₂₆H₂₅F₂N₅O₂ | |
| 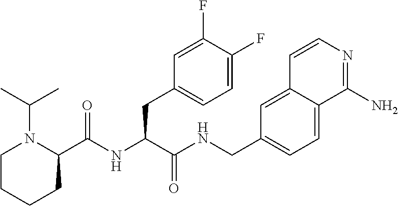 [SMILES: CC(C)N1CCCC[C@@H]1C(=O)N[C@@H](Cc1ccc(F)c(F)c1)C(=O)NCc1ccc2c(N)nccc2c1] | 0.02 |
| C₂₈H₃₃F₂N₅O₂ | |
| 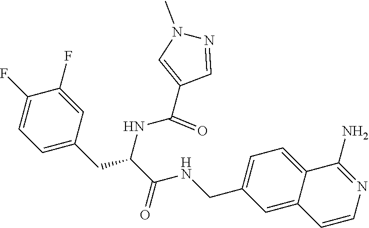 [SMILES: Cn1cc(C(=O)N[C@@H](Cc2ccc(F)c(F)c2)C(=O)NCc2ccc3c(N)nccc3c2)cn1] | 0.03 |
| C₂₄H₂₂F₂N₆O₂ | |
| 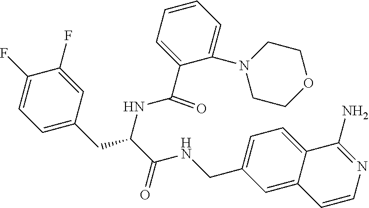 [SMILES: Nc1nccc2cc(CNC(=O)[C@H](Cc3ccc(F)c(F)c3)NC(=O)c3ccccc3N3CCOCC3)ccc12] | 0.04 |
| C₃₀H₂₉F₂N₅O₃ | |
| 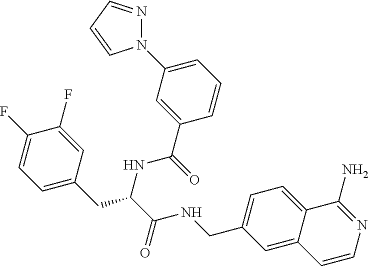 [SMILES: Nc1nccc2cc(CNC(=O)[C@H](Cc3ccc(F)c(F)c3)NC(=O)c3cccc(-n4cccn4)c3)ccc12] | 0.05 |
| C₂₉H₂₄F₂N₆O₂ | |
| 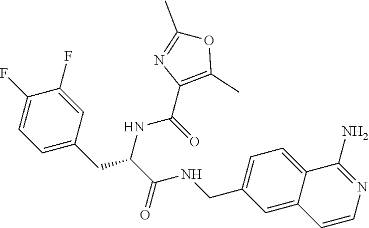 [SMILES: Cc1nc(C(=O)N[C@@H](Cc2ccc(F)c(F)c2)C(=O)NCc2ccc3c(N)nccc3c2)c(C)o1] | 0.06 |
| C₂₅H₂₃F₂N₅O₃ | |
| 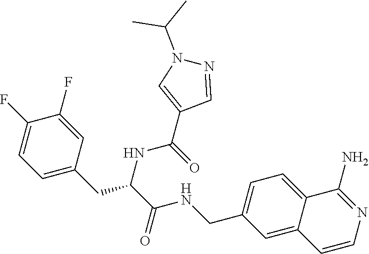 [SMILES: CC(C)n1cc(C(=O)N[C@@H](Cc2ccc(F)c(F)c2)C(=O)NCc2ccc3c(N)nccc3c2)cn1] | 0.07 |
| C₂₆H₂₆F₂N₆O₂ | |
| 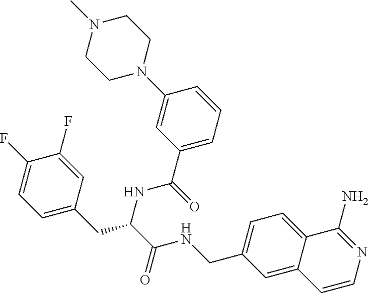 [SMILES: CN1CCN(c2cccc(C(=O)N[C@@H](Cc3ccc(F)c(F)c3)C(=O)NCc3ccc4c(N)nccc4c3)c2)CC1] | 0.08 |
| C₃₁H₃₂F₂N₆O₂ | |
| 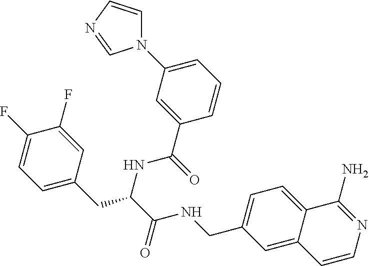 [SMILES: Nc1nccc2cc(CNC(=O)[C@H](Cc3ccc(F)c(F)c3)NC(=O)c3cccc(-n4ccnc4)c3)ccc12] | 0.09 |
| C₂₉H₂₄F₂N₆O₂ | |
| 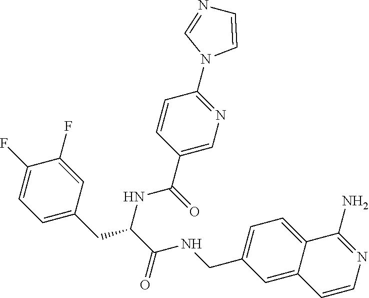 [SMILES: Nc1nccc2cc(CNC(=O)[C@H](Cc3ccc(F)c(F)c3)NC(=O)c3ccc(-n4ccnc4)nc3)ccc12] | 0.10 |
| C₂₈H₂₃F₂N₇O₂ | |
| 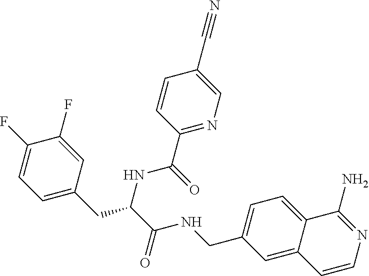 [SMILES: N#Cc1ccc(C(=O)N[C@@H](Cc2ccc(F)c(F)c2)C(=O)NCc2ccc3c(N)nccc3c2)nc1] | 0.11 |
| C₂₆H₂₀F₂N₆O₂ | |
| 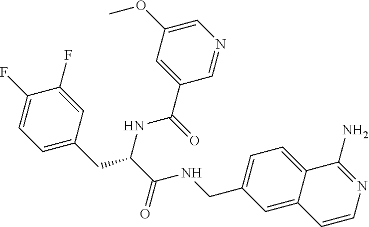 [SMILES: COc1cncc(C(=O)N[C@@H](Cc2ccc(F)c(F)c2)C(=O)NCc2ccc3c(N)nccc3c2)c1] | 0.12 |
| C₂₆H₂₃F₂N₅O₃ | |
| 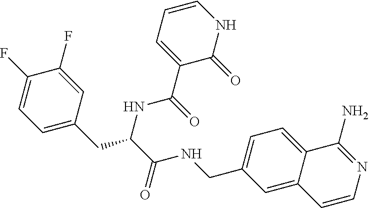 [SMILES: Nc1nccc2cc(CNC(=O)[C@H](Cc3ccc(F)c(F)c3)NC(=O)c3ccc[nH]c3=O)ccc12] | 0.13 |
| C₂₅H₂₁F₂N₅O₃ | |
| 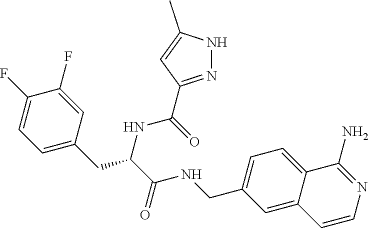 [SMILES: Cc1cc(C(=O)N[C@@H](Cc2ccc(F)c(F)c2)C(=O)NCc2ccc3c(N)nccc3c2)n[nH]1] | 0.14 |
| C₂₄H₂₂F₂N₆O₂ | |
| 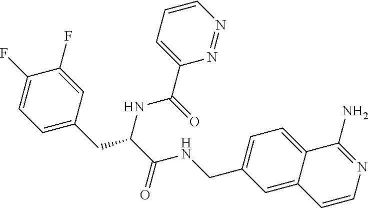 [SMILES: Nc1nccc2cc(CNC(=O)[C@H](Cc3ccc(F)c(F)c3)NC(=O)c3cccnn3)ccc12] | 0.15 |
| C₂₄H₂₀F₂N₆O₂ | |
| 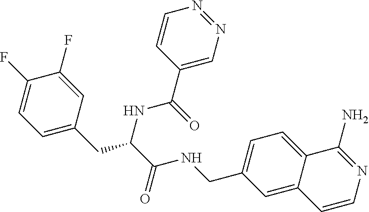 [SMILES: Nc1nccc2cc(CNC(=O)[C@H](Cc3ccc(F)c(F)c3)NC(=O)c3ccnnc3)ccc12] | 0.16 |
| C₂₄H₂₀F₂N₆O₂ | |
| 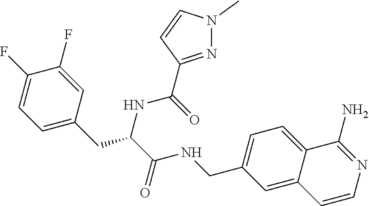 [SMILES: Cn1ccc(C(=O)N[C@@H](Cc2ccc(F)c(F)c2)C(=O)NCc2ccc3c(N)nccc3c2)n1] | 0.17 |
| C₂₄H₂₂F₂N₆O₂ | |
| 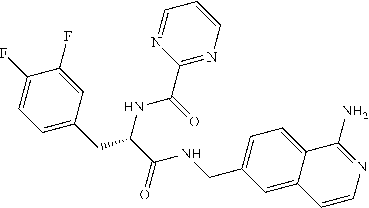 [SMILES: Nc1nccc2cc(CNC(=O)[C@H](Cc3ccc(F)c(F)c3)NC(=O)c3ncccn3)ccc12] | 0.18 |
| C₂₄H₂₀F₂N₆O₂ | |
| 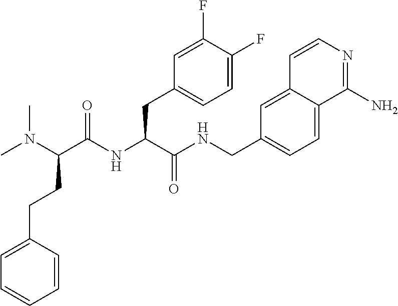 [SMILES: CN(C)[C@H](CCc1ccccc1)C(=O)N[C@@H](Cc1ccc(F)c(F)c1)C(=O)NCc1ccc2c(N)nccc2c1] | 0.19 |
| C₃₁H₃₃F₂N₅O₂ | |
| 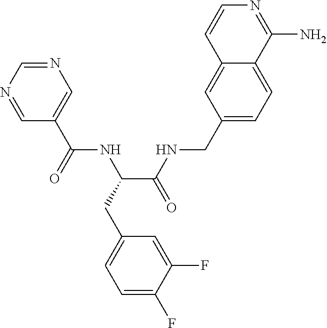 [SMILES: Nc1nccc2cc(CNC(=O)[C@H](Cc3ccc(F)c(F)c3)NC(=O)c3cncnc3)ccc12] | 0.20 |
| C₂₄H₂₀F₂N₆O₂ | |
| 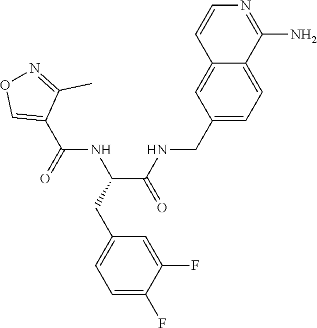 [SMILES: Cc1nocc1C(=O)N[C@@H](Cc1ccc(F)c(F)c1)C(=O)NCc1ccc2c(N)nccc2c1] | 0.21 |
| C₂₄H₂₁F₂N₅O₃ | |
| 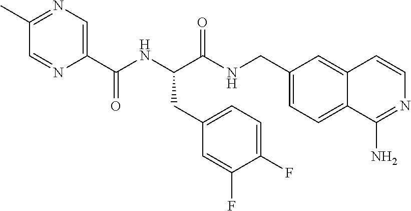 [SMILES: Cc1cnc(C(=O)N[C@@H](Cc2ccc(F)c(F)c2)C(=O)NCc2ccc3c(N)nccc3c2)cn1] | 0.22 |
| C₂₅H₂₂F₂N₆O₂ | |
| 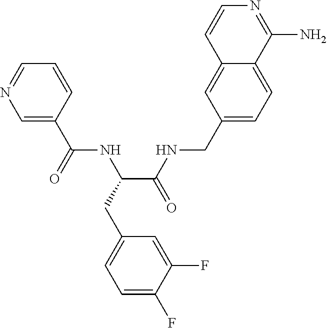 [SMILES: Nc1nccc2cc(CNC(=O)[C@H](Cc3ccc(F)c(F)c3)NC(=O)c3cccnc3)ccc12] | 0.23 |
| C₂₅H₂₁F₂N₅O₂ | |
| 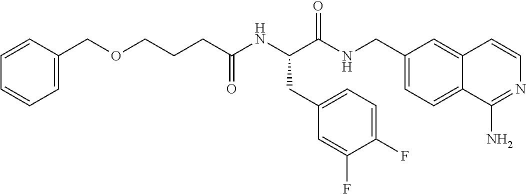 [SMILES: Nc1nccc2cc(CNC(=O)[C@H](Cc3ccc(F)c(F)c3)NC(=O)CCCOCc3ccccc3)ccc12] | 0.24 |
| C₃₀H₃₀F₂N₄O₃ | |
| 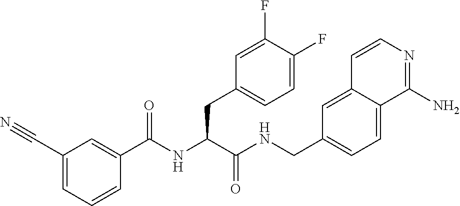 [SMILES: N#Cc1cccc(C(=O)N[C@@H](Cc2ccc(F)c(F)c2)C(=O)NCc2ccc3c(N)nccc3c2)c1] | 0.25 |
| C₂₇H₂₁F₂N₅O₂ | |
| 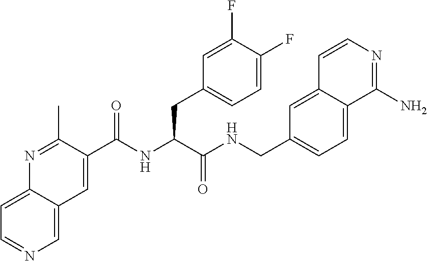 [SMILES: Cc1nc2ccncc2cc1C(=O)N[C@@H](Cc1ccc(F)c(F)c1)C(=O)NCc1ccc2c(N)nccc2c1] | 0.26 |
| C₂₉H₂₄F₂N₆O₂ | |
| 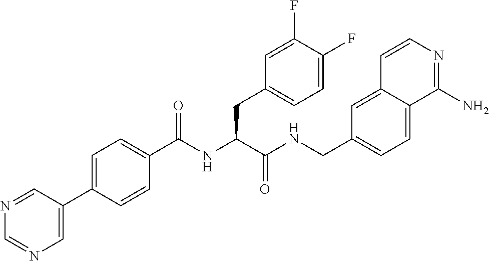 [SMILES: Nc1nccc2cc(CNC(=O)[C@H](Cc3ccc(F)c(F)c3)NC(=O)c3ccc(-c4cncnc4)cc3)ccc12] | 0.27 |
| C₃₀H₂₄F₂N₆O₂ | |
| 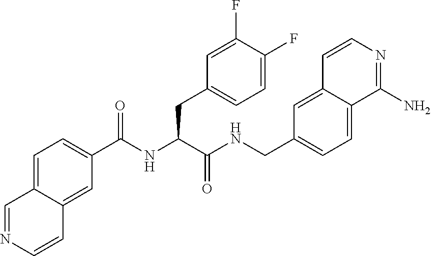 [SMILES: Nc1nccc2cc(CNC(=O)[C@H](Cc3ccc(F)c(F)c3)NC(=O)c3ccc4cnccc4c3)ccc12] | 0.28 |
| C₂₉H₂₃F₂N₅O₂ | |
| 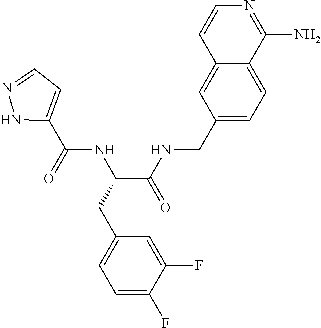 [SMILES: Nc1nccc2cc(CNC(=O)[C@H](Cc3ccc(F)c(F)c3)NC(=O)c3ccn[nH]3)ccc12] | 0.29 |
| C₂₃H₂₀F₂N₆O₂ | |
| 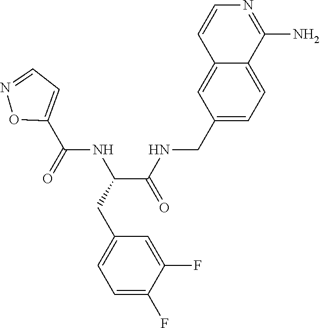 [SMILES: Nc1nccc2cc(CNC(=O)[C@H](Cc3ccc(F)c(F)c3)NC(=O)c3ccno3)ccc12] | 0.30 |
| C₂₃H₁₉F₂N₅O₃ | |
| 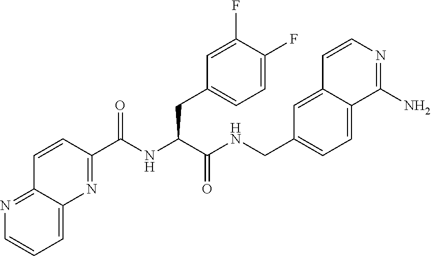 [SMILES: Nc1nccc2cc(CNC(=O)[C@H](Cc3ccc(F)c(F)c3)NC(=O)c3ccc4ncccc4n3)ccc12] | 0.31 |
| C₂₈H₂₂F₂N₆O₂ | |
| 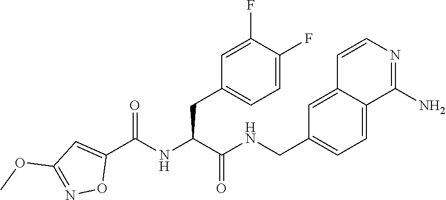 [SMILES: COc1cc(C(=O)N[C@@H](Cc2ccc(F)c(F)c2)C(=O)NCc2ccc3c(N)nccc3c2)on1] | 0.32 |
| C₂₄H₂₁F₂N₅O₄ | |
| 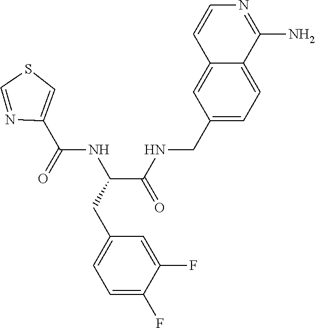 [SMILES: Nc1nccc2cc(CNC(=O)[C@H](Cc3ccc(F)c(F)c3)NC(=O)c3cscn3)ccc12] | 0.33 |
| C₂₃H₁₉F₂N₅O₂S | |
| 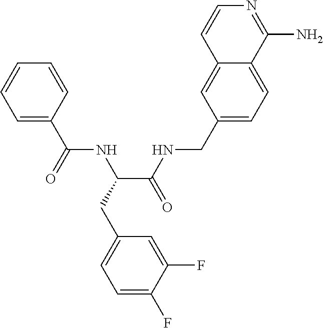 [SMILES: Nc1nccc2cc(CNC(=O)[C@H](Cc3ccc(F)c(F)c3)NC(=O)c3ccccc3)ccc12] | 0.34 |
| C₂₆H₂₂F₂N₄O₂ | |
| 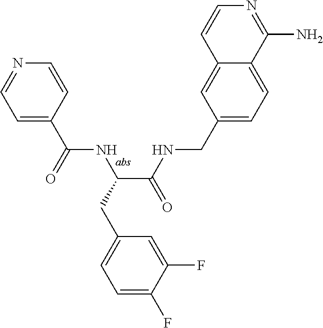 [SMILES: Nc1nccc2cc(CNC(=O)[C@H](Cc3ccc(F)c(F)c3)NC(=O)c3ccncc3)ccc12] | 0.35 |
| C₂₅H₂₁F₂N₅O₂ | |
| 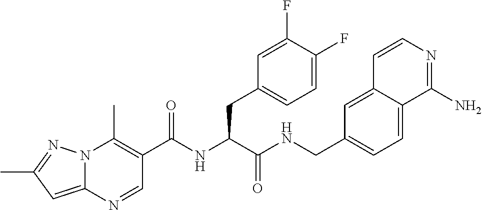 [SMILES: Cc1cc2ncc(C(=O)N[C@@H](Cc3ccc(F)c(F)c3)C(=O)NCc3ccc4c(N)nccc4c3)c(C)n2n1] | 0.36 |
| C₂₈H₂₅F₂N₇O₂ | |
| 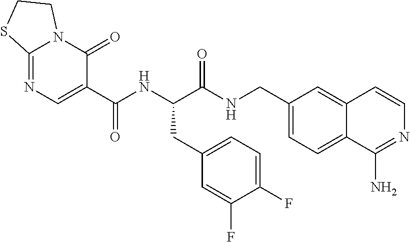 [SMILES: Nc1nccc2cc(CNC(=O)[C@H](Cc3ccc(F)c(F)c3)NC(=O)c3cnc4n(c3=O)CCS4)ccc12] | 0.37 |
| C₂₆H₂₂F₂N₆O₃S | |
| 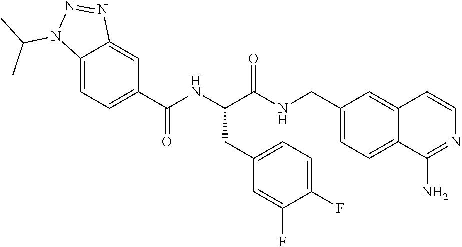 [SMILES: CC(C)n1nnc2cc(C(=O)N[C@@H](Cc3ccc(F)c(F)c3)C(=O)NCc3ccc4c(N)nccc4c3)ccc21] | 0.38 |
| C₂₉H₂₇F₂N₇O₂ | |
| 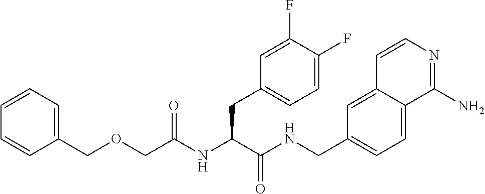 [SMILES: Nc1nccc2cc(CNC(=O)[C@H](Cc3ccc(F)c(F)c3)NC(=O)COCc3ccccc3)ccc12] | 0.39 |
| C₂₈H₂₆F₂N₄O₃ | |
| 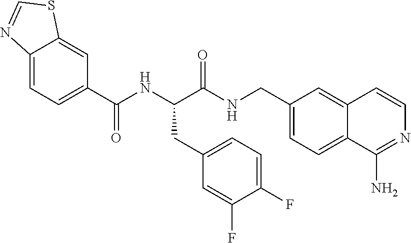 [SMILES: Nc1nccc2cc(CNC(=O)[C@H](Cc3ccc(F)c(F)c3)NC(=O)c3ccc4ncsc4c3)ccc12] | 0.40 |
| C₂₇H₂₁F₂N₅O₂S | |
| 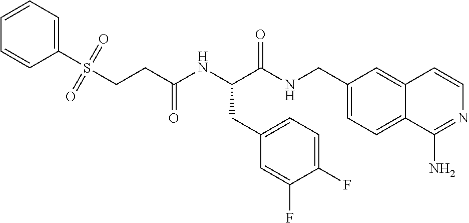 [SMILES: Nc1nccc2cc(CNC(=O)[C@H](Cc3ccc(F)c(F)c3)NC(=O)CCS(=O)(=O)c3ccccc3)ccc12] | 0.41 |
| C₂₈H₂₆F₂N₄O₄S | |
| 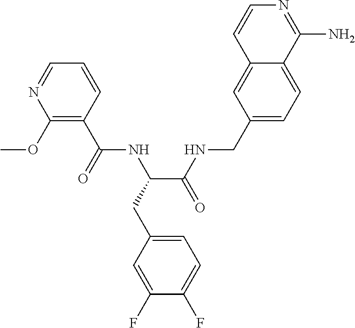 [SMILES: COc1ncccc1C(=O)N[C@@H](Cc1ccc(F)c(F)c1)C(=O)NCc1ccc2c(N)nccc2c1] | 0.42 |
| C₂₆H₂₃F₂N₅O₃ | |
| 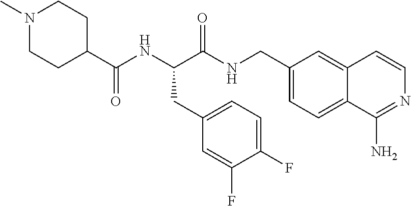 [SMILES: CN1CCC(C(=O)N[C@@H](Cc2ccc(F)c(F)c2)C(=O)NCc2ccc3c(N)nccc3c2)CC1] | 0.43 |
| C₂₆H₂₉F₂N₅O₂ | |
| 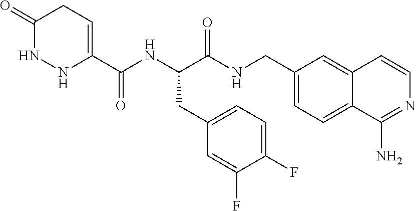 [SMILES: Nc1nccc2cc(CNC(=O)[C@H](Cc3ccc(F)c(F)c3)NC(=O)C3=CCC(=O)NN3)ccc12] | 0.44 |
| C₂₄H₂₂F₂N₆O₃ | |
| 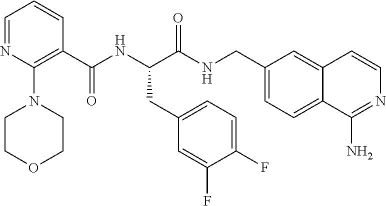 [SMILES: Nc1nccc2cc(CNC(=O)[C@H](Cc3ccc(F)c(F)c3)NC(=O)c3cccnc3N3CCOCC3)ccc12] | 0.45 |
| C₂₉H₂₈F₂N₆O₃ | |
| 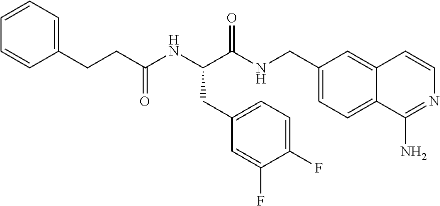 [SMILES: Nc1nccc2cc(CNC(=O)[C@H](Cc3ccc(F)c(F)c3)NC(=O)CCc3ccccc3)ccc12] | 0.46 |
| C₂₈H₂₆F₂N₄O₂ | |
| 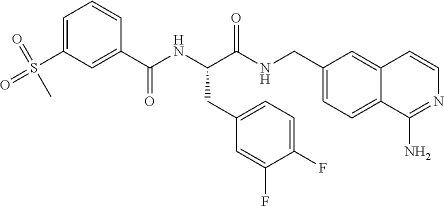 [SMILES: CS(=O)(=O)c1cccc(C(=O)N[C@@H](Cc2ccc(F)c(F)c2)C(=O)NCc2ccc3c(N)nccc3c2)c1] | 0.47 |
| C₂₇H₂₄F₂N₄O₄S | |
| 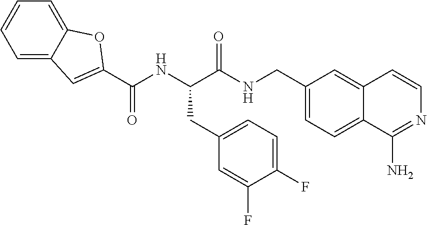 [SMILES: Nc1nccc2cc(CNC(=O)[C@H](Cc3ccc(F)c(F)c3)NC(=O)c3cc4ccccc4o3)ccc12] | 0.48 |
| C₂₈H₂₂F₂N₄O₃ | |
| 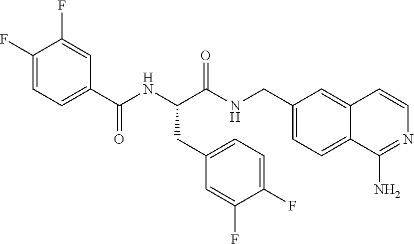 [SMILES: Nc1nccc2cc(CNC(=O)[C@H](Cc3ccc(F)c(F)c3)NC(=O)c3ccc(F)c(F)c3)ccc12] | 0.49 |
| C₂₆H₂₀F₄N₄O₂ | |
| 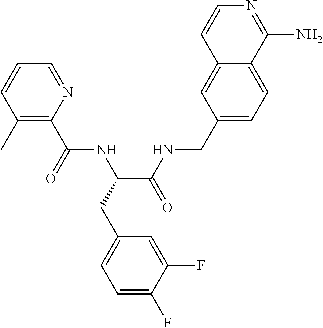 [SMILES: Cc1cccnc1C(=O)N[C@@H](Cc1ccc(F)c(F)c1)C(=O)NCc1ccc2c(N)nccc2c1] | 0.50 |
| C₂₆H₂₃F₂N₅O₂ | |
| 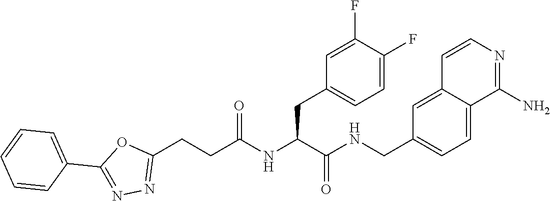 [SMILES: Nc1nccc2cc(CNC(=O)[C@H](Cc3ccc(F)c(F)c3)NC(=O)CCc3nnc(-c4ccccc4)o3)ccc12] | 0.51 |
| C₃₀H₂₆F₂N₆O₃ | |
| 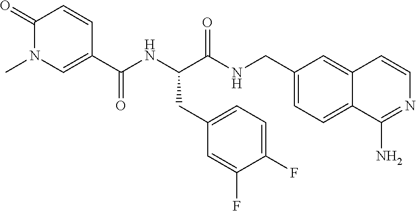 [SMILES: Cn1cc(C(=O)N[C@@H](Cc2ccc(F)c(F)c2)C(=O)NCc2ccc3c(N)nccc3c2)ccc1=O] | 0.52 |
| C₂₆H₂₃F₂N₅O₃ | |
| 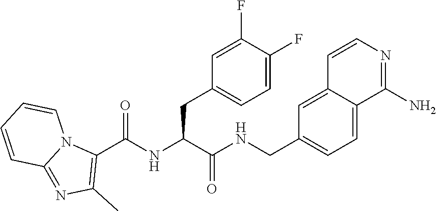 [SMILES: Cc1nc2ccccn2c1C(=O)N[C@@H](Cc1ccc(F)c(F)c1)C(=O)NCc1ccc2c(N)nccc2c1] | 0.53 |
| C₂₈H₂₄F₂N₆O₂ | |
| 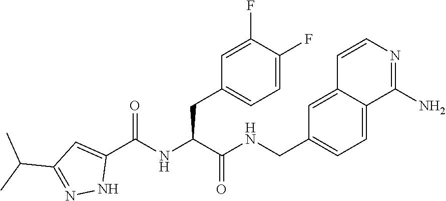 [SMILES: CC(C)c1cc(C(=O)N[C@@H](Cc2ccc(F)c(F)c2)C(=O)NCc2ccc3c(N)nccc3c2)[nH]n1] | 0.54 |
| C₂₆H₂₆F₂N₆O₂ | |
| 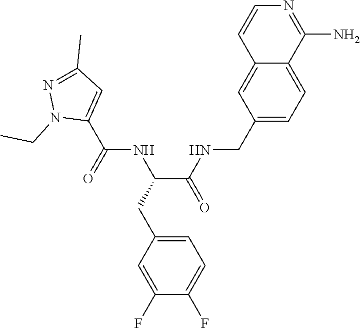 [SMILES: CCn1nc(C)cc1C(=O)N[C@@H](Cc1ccc(F)c(F)c1)C(=O)NCc1ccc2c(N)nccc2c1] | 0.55 |
| C₂₆H₂₆F₂N₆O₂ | |
| 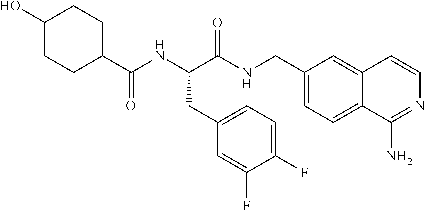 [SMILES: Nc1nccc2cc(CNC(=O)[C@H](Cc3ccc(F)c(F)c3)NC(=O)C3CCC(O)CC3)ccc12] | 0.56 |
| C₂₆H₂₈F₂N₄O₃ | |
| 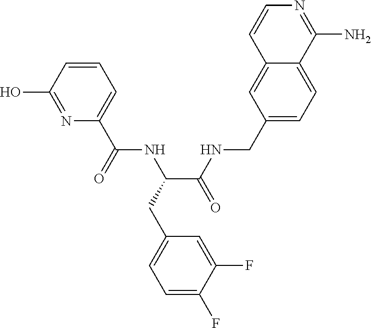 [SMILES: Nc1nccc2cc(CNC(=O)[C@H](Cc3ccc(F)c(F)c3)NC(=O)c3cccc(O)n3)ccc12] | 0.57 |
| C₂₅H₂₁F₂N₅O₃ | |
| 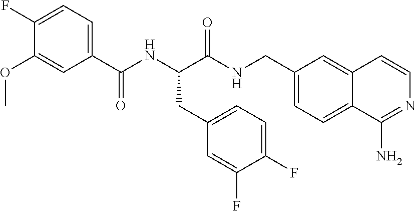 [SMILES: COc1cc(C(=O)N[C@@H](Cc2ccc(F)c(F)c2)C(=O)NCc2ccc3c(N)nccc3c2)ccc1F] | 0.58 |
| C₂₇H₂₃F₃N₄O₃ | |
| 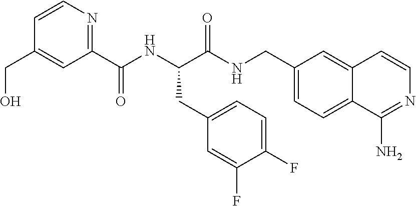 [SMILES: Nc1nccc2cc(CNC(=O)[C@H](Cc3ccc(F)c(F)c3)NC(=O)c3cc(CO)ccn3)ccc12] | 0.59 |
| C₂₆H₂₃F₂N₅O₃ | |
| 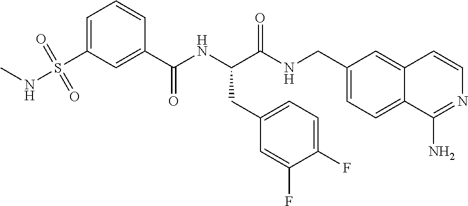 [SMILES: CNS(=O)(=O)c1cccc(C(=O)N[C@@H](Cc2ccc(F)c(F)c2)C(=O)NCc2ccc3c(N)nccc3c2)c1] | 0.60 |
| C₂₇H₂₅F₂N₅O₄S | |
| 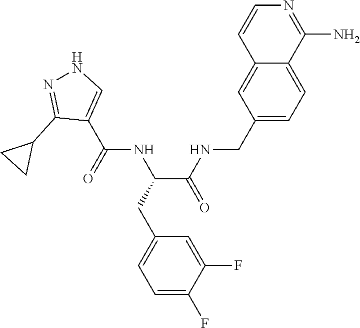 [SMILES: Nc1nccc2cc(CNC(=O)[C@H](Cc3ccc(F)c(F)c3)NC(=O)c3c[nH]nc3C3CC3)ccc12] | 0.61 |
| C₂₆H₂₄F₂N₆O₂ | |
| 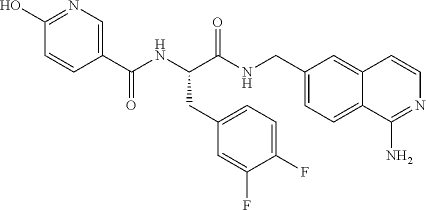 [SMILES: Nc1nccc2cc(CNC(=O)[C@H](Cc3ccc(F)c(F)c3)NC(=O)c3ccc(O)nc3)ccc12] | 0.62 |
| C₂₅H₂₁F₂N₅O₃ | |
| 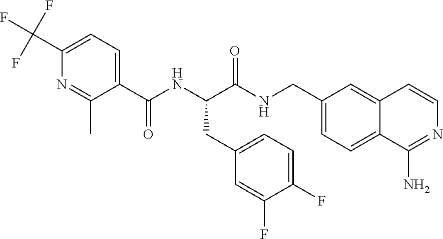 [SMILES: Cc1nc(C(F)(F)F)ccc1C(=O)N[C@@H](Cc1ccc(F)c(F)c1)C(=O)NCc1ccc2c(N)nccc2c1] | 0.63 |
| C₂₇H₂₂F₅N₅O₂ | |
| 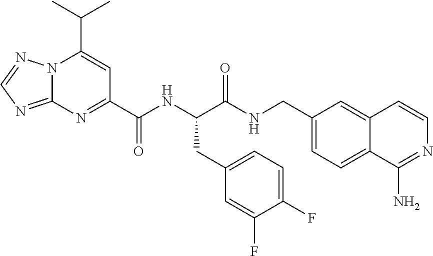 [SMILES: CC(C)c1cc(C(=O)N[C@@H](Cc2ccc(F)c(F)c2)C(=O)NCc2ccc3c(N)nccc3c2)nc2ncnn12] | 0.64 |
| C₂₈H₂₆F₂N₈O₂ | |
| 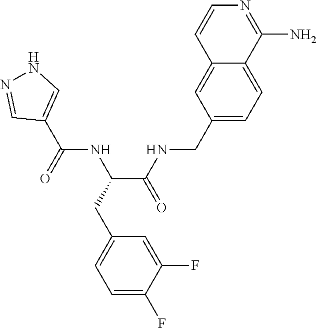 [SMILES: Nc1nccc2cc(CNC(=O)[C@H](Cc3ccc(F)c(F)c3)NC(=O)c3cn[nH]c3)ccc12] | 0.65 |
| C₂₃H₂₀F₂N₆O₂ | |
| 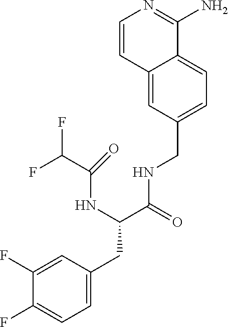 [SMILES: Nc1nccc2cc(CNC(=O)[C@H](Cc3ccc(F)c(F)c3)NC(=O)C(F)F)ccc12] | 0.66 |
| C₂₁H₁₈F₄N₄O₂ | |
| 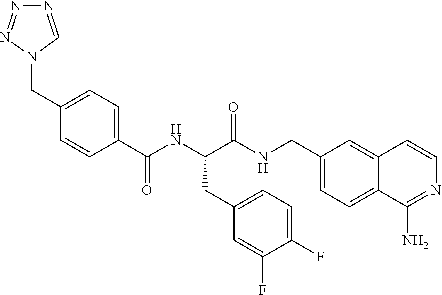 [SMILES: Nc1nccc2cc(CNC(=O)[C@H](Cc3ccc(F)c(F)c3)NC(=O)c3ccc(Cn4cnnn4)cc3)ccc12] | 0.67 |
| C₂₈H₂₄F₂N₈O₂ | |
| 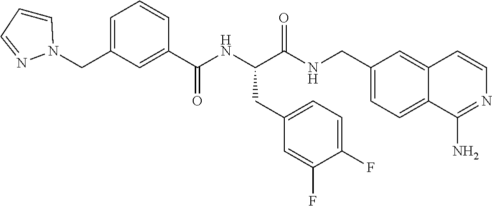 [SMILES: Nc1nccc2cc(CNC(=O)[C@H](Cc3ccc(F)c(F)c3)NC(=O)c3cccc(Cn4cccn4)c3)ccc12] | 0.68 |
| C₃₀H₂₆F₂N₆O₂ | |
| 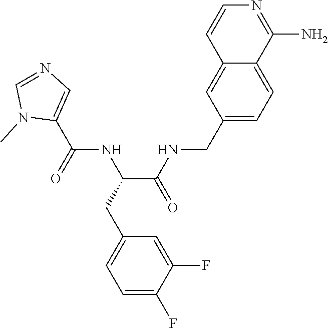 [SMILES: Cn1cncc1C(=O)N[C@@H](Cc1ccc(F)c(F)c1)C(=O)NCc1ccc2c(N)nccc2c1] | 0.69 |
| C₂₄H₂₂F₂N₆O₂ | |
| 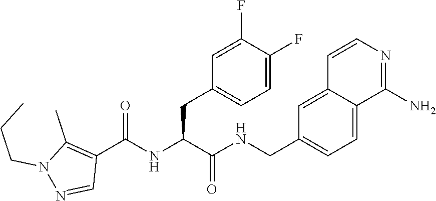 [SMILES: CCCn1ncc(C(=O)N[C@@H](Cc2ccc(F)c(F)c2)C(=O)NCc2ccc3c(N)nccc3c2)c1C] | 0.70 |
| C₂₇H₂₈F₂N₆O₂ | |
| 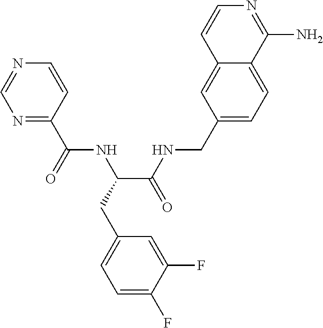 [SMILES: Nc1nccc2cc(CNC(=O)[C@H](Cc3ccc(F)c(F)c3)NC(=O)c3ccncn3)ccc12] | 0.71 |
| C₂₄H₂₀F₂N₆O₂ | |
| 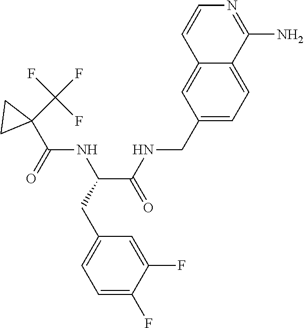 [SMILES: Nc1nccc2cc(CNC(=O)[C@H](Cc3ccc(F)c(F)c3)NC(=O)C3(C(F)(F)F)CC3)ccc12] | 0.72 |
| C₂₄H₂₁F₅N₄O₂ | |
| 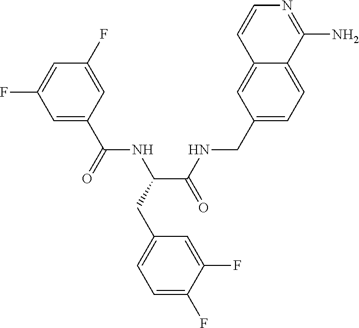 [SMILES: Nc1nccc2cc(CNC(=O)[C@H](Cc3ccc(F)c(F)c3)NC(=O)c3cc(F)cc(F)c3)ccc12] | 0.73 |
| C₂₆H₂₀F₄N₄O₂ | |
| 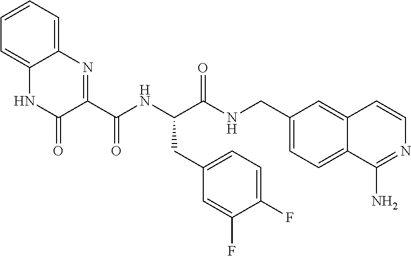 [SMILES: Nc1nccc2cc(CNC(=O)[C@H](Cc3ccc(F)c(F)c3)NC(=O)c3nc4ccccc4[nH]c3=O)ccc12] | 0.74 |
| C₂₈H₂₂F₂N₆O₃ | |
| 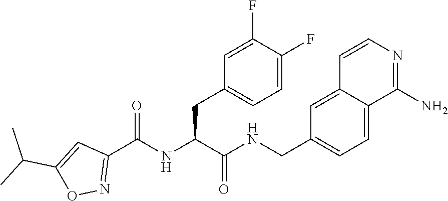 [SMILES: CC(C)c1cc(C(=O)N[C@@H](Cc2ccc(F)c(F)c2)C(=O)NCc2ccc3c(N)nccc3c2)no1] | 0.75 |
| C₂₆H₂₅F₂N₅O₃ | |
| 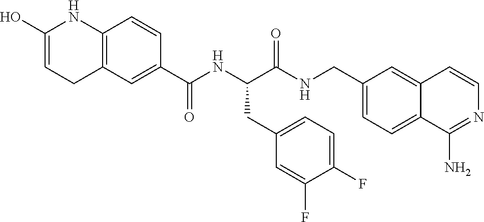 [SMILES: Nc1nccc2cc(CNC(=O)[C@H](Cc3ccc(F)c(F)c3)NC(=O)c3ccc4c(c3)CC=C(O)N4)ccc12] | 0.76 |
| C₂₉H₂₅F₂N₅O₃ | |
| 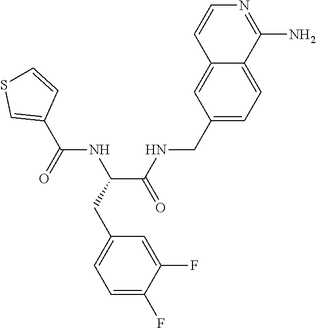 [SMILES: Nc1nccc2cc(CNC(=O)[C@H](Cc3ccc(F)c(F)c3)NC(=O)c3ccsc3)ccc12] | 0.77 |
| C₂₄H₂₀F₂N₄O₂S | |
| 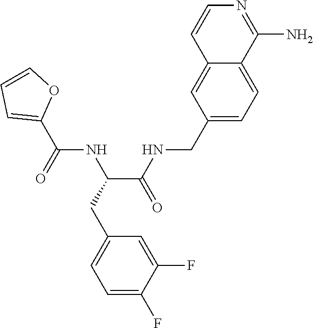 [SMILES: Nc1nccc2cc(CNC(=O)[C@H](Cc3ccc(F)c(F)c3)NC(=O)c3ccco3)ccc12] | 0.78 |
| C₂₄H₂₀F₂N₄O₃ | |
| 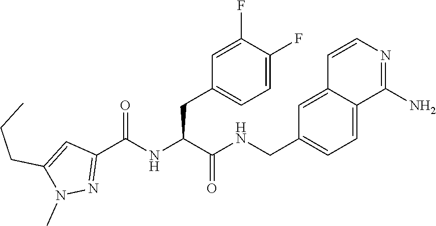 [SMILES: CCCc1cc(C(=O)N[C@@H](Cc2ccc(F)c(F)c2)C(=O)NCc2ccc3c(N)nccc3c2)nn1C] | 0.79 |
| C₂₇H₂₈F₂N₆O₂ | |
| 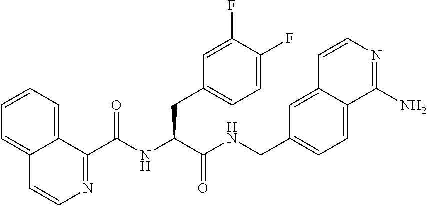 [SMILES: Nc1nccc2cc(CNC(=O)[C@H](Cc3ccc(F)c(F)c3)NC(=O)c3nccc4ccccc34)ccc12] | 0.80 |
| C₂₉H₂₃F₂N₅O₂ | |
| 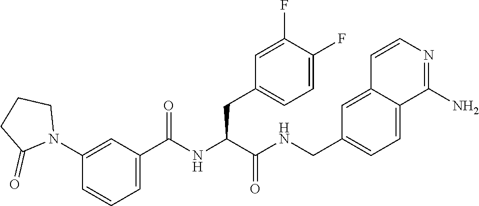 [SMILES: Nc1nccc2cc(CNC(=O)[C@H](Cc3ccc(F)c(F)c3)NC(=O)c3cccc(N4CCCC4=O)c3)ccc12] | 0.81 |
| C₃₀H₂₇F₂N₅O₃ | |
| 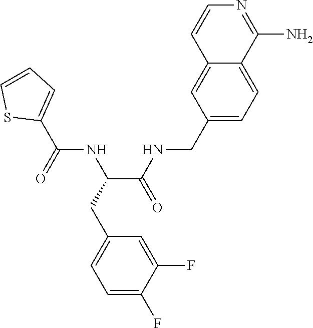 [SMILES: Nc1nccc2cc(CNC(=O)[C@H](Cc3ccc(F)c(F)c3)NC(=O)c3cccs3)ccc12] | 0.82 |
| C₂₄H₂₀F₂N₄O₂S | |
| 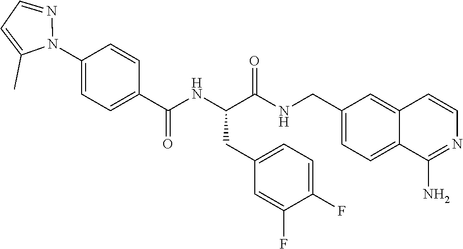 [SMILES: Cc1ccnn1-c1ccc(C(=O)N[C@@H](Cc2ccc(F)c(F)c2)C(=O)NCc2ccc3c(N)nccc3c2)cc1] | 0.83 |
| C₃₀H₂₆F₂N₆O₂ | |
| 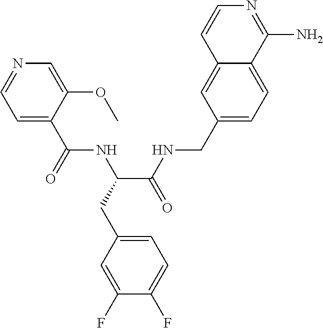 [SMILES: COc1cnccc1C(=O)N[C@@H](Cc1ccc(F)c(F)c1)C(=O)NCc1ccc2c(N)nccc2c1] | 0.84 |
| C₂₆H₂₃F₂N₅O₃ | |
| 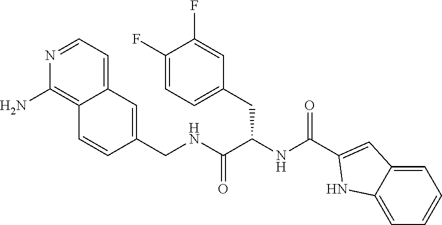 [SMILES: Nc1nccc2cc(CNC(=O)[C@H](Cc3ccc(F)c(F)c3)NC(=O)c3cc4ccccc4[nH]3)ccc12] | 0.85 |
| C₂₈H₂₃F₂N₅O₂ | |
| 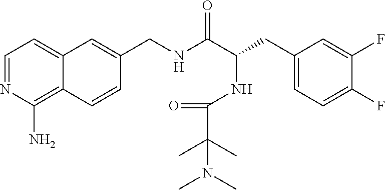 [SMILES: CN(C)C(C)(C)C(=O)N[C@@H](Cc1ccc(F)c(F)c1)C(=O)NCc1ccc2c(N)nccc2c1] | 0.86 |
| C₂₅H₂₉F₂N₅O₂ | |
| 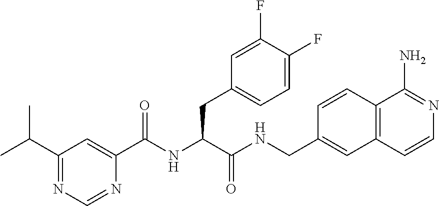 [SMILES: CC(C)c1cc(C(=O)N[C@@H](Cc2ccc(F)c(F)c2)C(=O)NCc2ccc3c(N)nccc3c2)ncn1] | 0.87 |
| C₂₇H₂₆F₂N₆O₂ | |
| 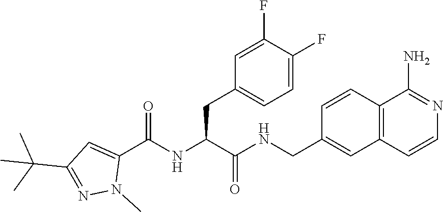 [SMILES: Cn1nc(C(C)(C)C)cc1C(=O)N[C@@H](Cc1ccc(F)c(F)c1)C(=O)NCc1ccc2c(N)nccc2c1] | 0.88 |
| C₂₈H₃₀F₂N₆O₂ | |
| 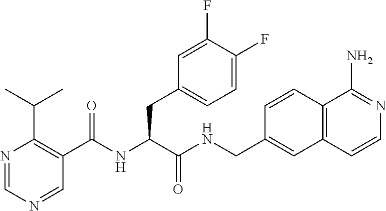 [SMILES: CC(C)c1ncncc1C(=O)N[C@@H](Cc1ccc(F)c(F)c1)C(=O)NCc1ccc2c(N)nccc2c1] | 0.89 |
| C₂₇H₂₆F₂N₆O₂ | |
| 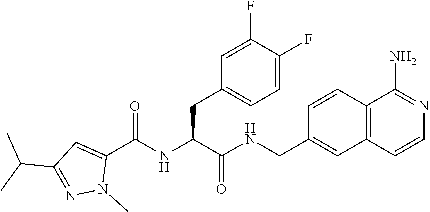 [SMILES: CC(C)c1cc(C(=O)N[C@@H](Cc2ccc(F)c(F)c2)C(=O)NCc2ccc3c(N)nccc3c2)n(C)n1] | 0.90 |
| C₂₇H₂₈F₂N₆O₂ | |
| 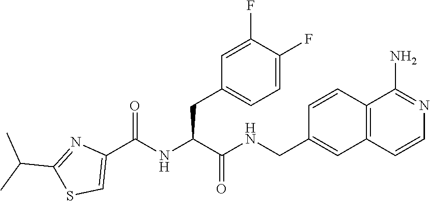 [SMILES: CC(C)c1nc(C(=O)N[C@@H](Cc2ccc(F)c(F)c2)C(=O)NCc2ccc3c(N)nccc3c2)cs1] | 0.91 |
| C₂₆H₂₅F₂N₅O₂S | |
| 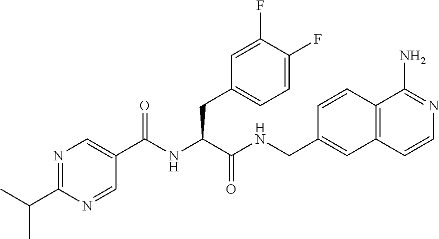 [SMILES: CC(C)c1ncc(C(=O)N[C@@H](Cc2ccc(F)c(F)c2)C(=O)NCc2ccc3c(N)nccc3c2)cn1] | 0.92 |
| C₂₇H₂₆F₂N₆O₂ | |
| 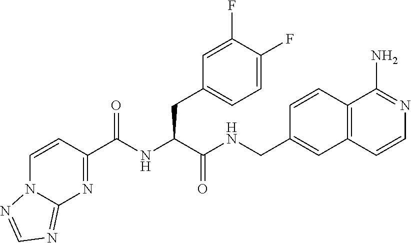 [SMILES: Nc1nccc2cc(CNC(=O)[C@H](Cc3ccc(F)c(F)c3)NC(=O)c3ccn4ncnc4n3)ccc12] | 0.93 |
| C₂₅H₂₀F₂N₈O₂ | |
| 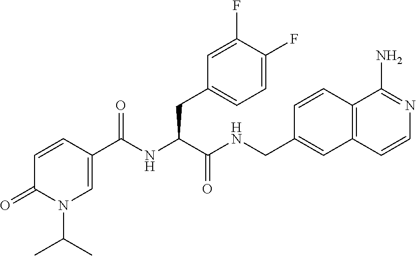 [SMILES: CC(C)n1cc(C(=O)N[C@@H](Cc2ccc(F)c(F)c2)C(=O)NCc2ccc3c(N)nccc3c2)ccc1=O] | 0.94 |
| C₂₈H₂₇F₂N₅O₃ | |
| 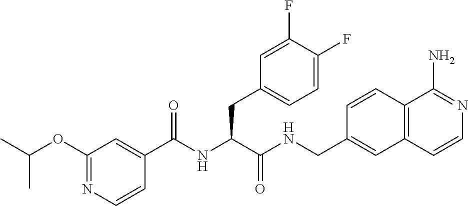 [SMILES: CC(C)Oc1cc(C(=O)N[C@@H](Cc2ccc(F)c(F)c2)C(=O)NCc2ccc3c(N)nccc3c2)ccn1] | 0.95 |
| C₂₈H₂₇F₂N₅O₃ | |
| 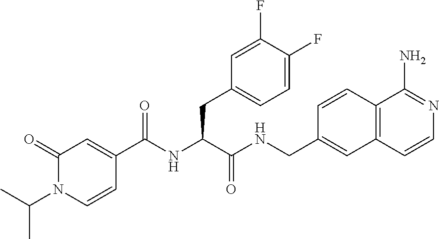 [SMILES: CC(C)n1ccc(C(=O)N[C@@H](Cc2ccc(F)c(F)c2)C(=O)NCc2ccc3c(N)nccc3c2)cc1=O] | 0.96 |
| C₂₈H₂₇F₂N₅O₃ | |
| 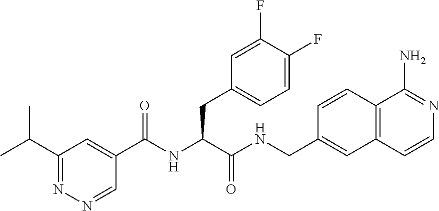 [SMILES: CC(C)c1cc(C(=O)N[C@@H](Cc2ccc(F)c(F)c2)C(=O)NCc2ccc3c(N)nccc3c2)cnn1] | 0.97 |
| C₂₇H₂₆F₂N₆O₂ | |
| 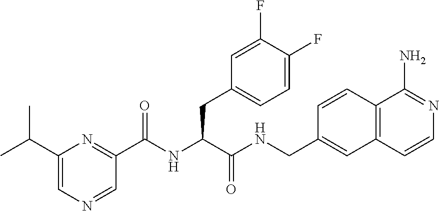 [SMILES: CC(C)c1cncc(C(=O)N[C@@H](Cc2ccc(F)c(F)c2)C(=O)NCc2ccc3c(N)nccc3c2)n1] | 0.98 |
| C₂₇H₂₆F₂N₆O₂ | |
| 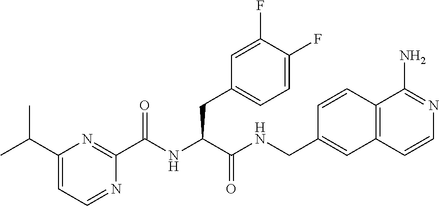 [SMILES: CC(C)c1ccnc(C(=O)N[C@@H](Cc2ccc(F)c(F)c2)C(=O)NCc2ccc3c(N)nccc3c2)n1] | 0.99 |
| C₂₇H₂₆F₂N₆O₂ | |

TABLE 2

| Structure | Example |
|---|---|
| Molecular formula | No. |
| 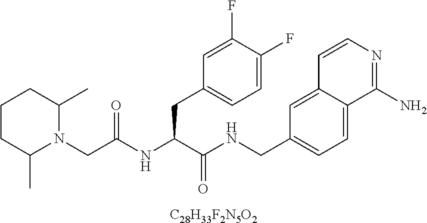 | 3.01 |
| 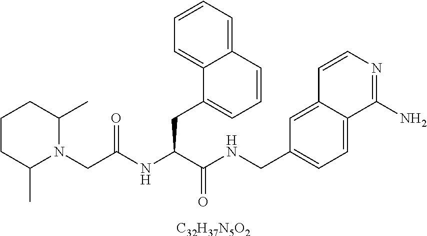 [SMILES: CC1CCCC(C)N1CC(=O)N[C@@H](Cc1cccc2ccccc12)C(=O)NCc1ccc2c(N)nccc2c1 ; "C32H37N5O2"] | 3.02 |
| 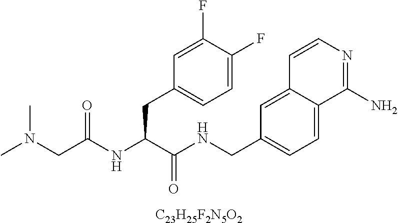 | 3.03 |
| 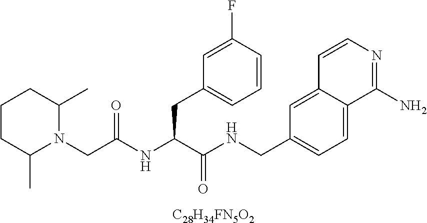 | 3.04 |
| 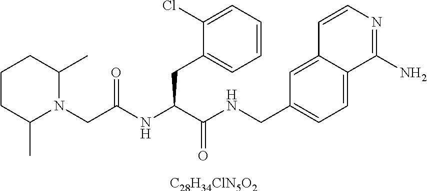 | 3.05 |
| 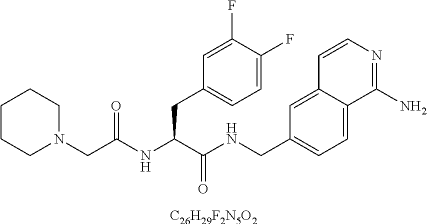 | 3.06 |
| 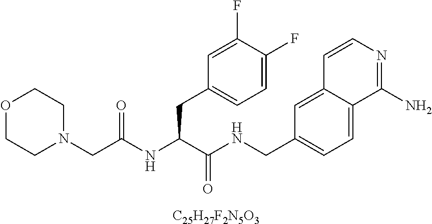 | 3.07 |
| 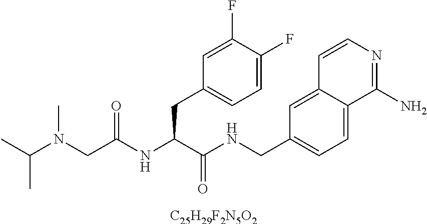 | 3.08 |
| 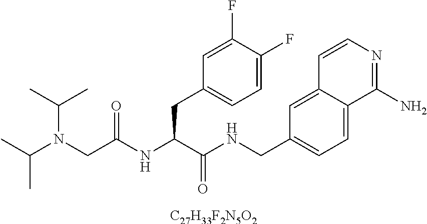 | 3.09 |
| 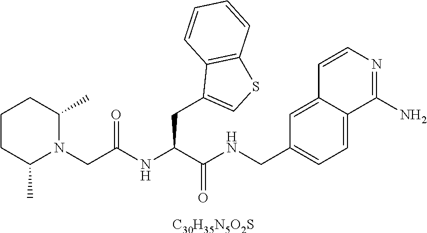 [SMILES: C[C@@H]1CCC[C@H](C)N1CC(=O)N[C@@H](Cc1csc2ccccc12)C(=O)NCc1ccc2c(N)nccc2c1 ; "C30H35N5O2S"] | 3.10 |
| 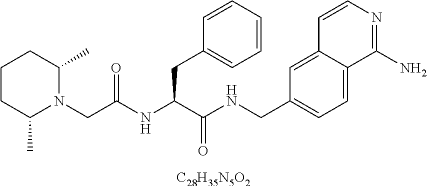 [SMILES: C[C@@H]1CCC[C@H](C)N1CC(=O)N[C@@H](Cc1ccccc1)C(=O)NCc1ccc2c(N)nccc2c1 ; "C28H35N5O2"] | 3.11 |
| 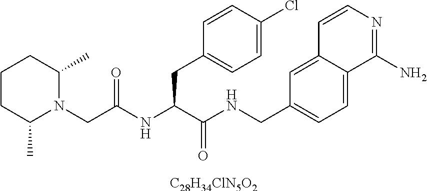 | 3.12 |
| 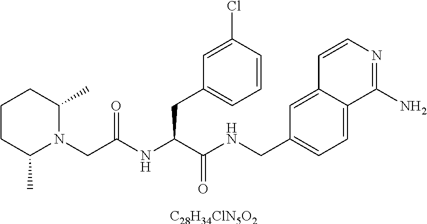 | 3.13 |
| 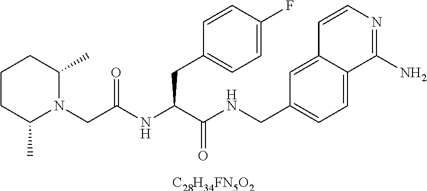 | 3.14 |
| 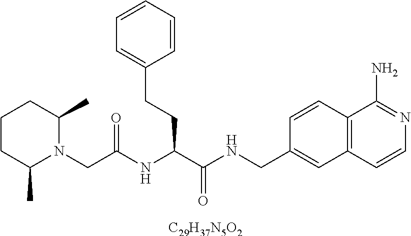 [SMILES: C[C@@H]1CCC[C@H](C)N1CC(=O)N[C@@H](CCc1ccccc1)C(=O)NCc1ccc2c(N)nccc2c1 ; "C29H37N5O2"] | 3.15 |
| 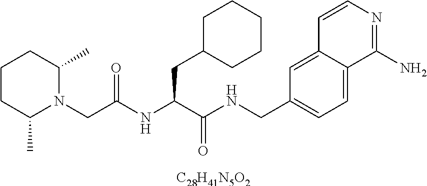 [SMILES: C[C@@H]1CCC[C@H](C)N1CC(=O)N[C@@H](CC1CCCCC1)C(=O)NCc1ccc2c(N)nccc2c1 ; "C28H41N5O2"] | 3.16 |
| 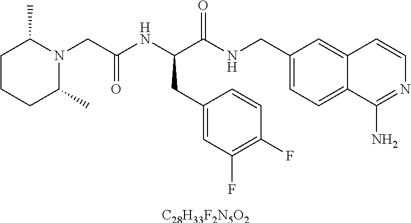 [SMILES: C[C@@H]1CCC[C@H](C)N1CC(=O)N[C@H](Cc1ccc(F)c(F)c1)C(=O)NCc1ccc2c(N)nccc2c1 ; "C28H33F2N5O2"] | 3.17 |
| 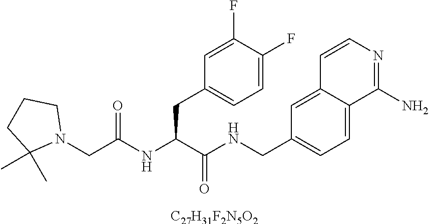 | 3.18 |
| 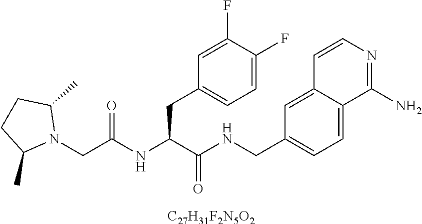 | 3.19 |
| 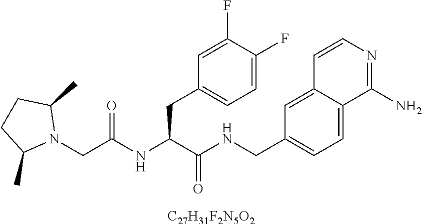 | 3.20 |
| 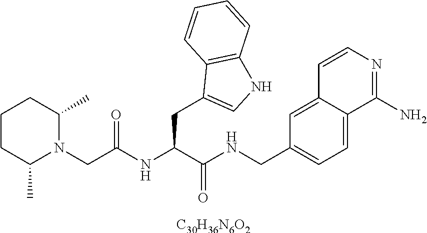 | 3.21 |
| 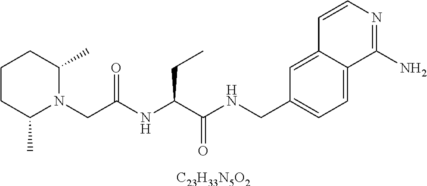 [SMILES: CC[C@H](NC(=O)CN1[C@H](C)CCC[C@@H]1C)C(=O)NCc1ccc2c(N)nccc2c1 ; "C23H33N5O2"] | 3.22 |
| 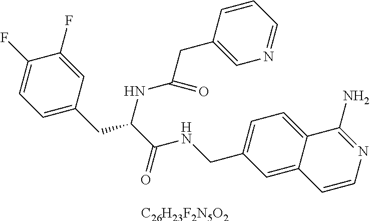 | 3.23 |
| 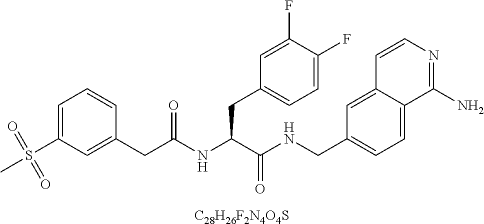 [SMILES: CS(=O)(=O)c1cccc(CC(=O)N[C@@H](Cc2ccc(F)c(F)c2)C(=O)NCc2ccc3c(N)nccc3c2)c1 ; "C28H26F2N4O4S"] | 3.24 |
| 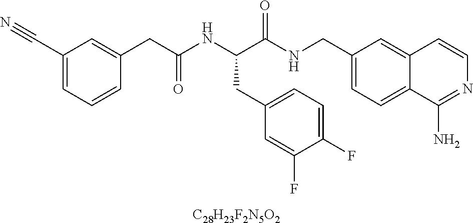 [SMILES: N#Cc1cccc(CC(=O)N[C@@H](Cc2ccc(F)c(F)c2)C(=O)NCc2ccc3c(N)nccc3c2)c1 ; "C28H23F2N5O2"] | 3.25 |
| 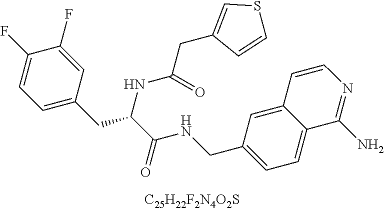 | 3.26 |
| 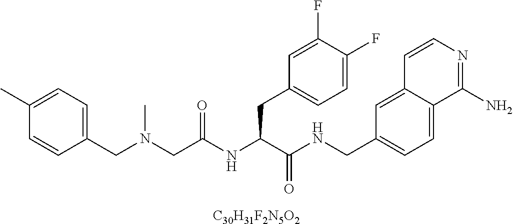 [SMILES: Cc1ccc(CN(C)CC(=O)N[C@@H](Cc2ccc(F)c(F)c2)C(=O)NCc2ccc3c(N)nccc3c2)cc1 ; "C30H31F2N5O2"] | 3.27 |
| 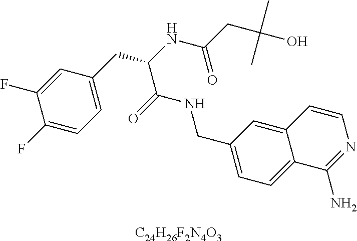 [SMILES: CC(C)(O)CC(=O)N[C@@H](Cc1ccc(F)c(F)c1)C(=O)NCc1ccc2c(N)nccc2c1 ; "C24H26F2N4O3"] | 3.28 |
| 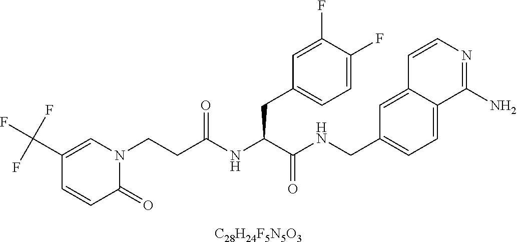 [SMILES: Nc1nccc2cc(CNC(=O)[C@H](Cc3ccc(F)c(F)c3)NC(=O)CCn3cc(C(F)(F)F)ccc3=O)ccc12 ; "C28H24F5N5O3"] | 3.29 |
| 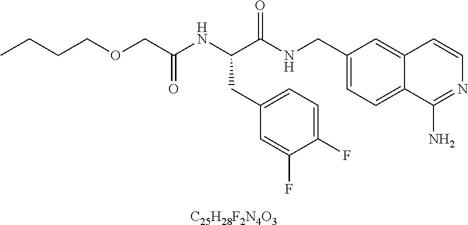 [SMILES: CCCCOCC(=O)N[C@@H](Cc1ccc(F)c(F)c1)C(=O)NCc1ccc2c(N)nccc2c1 ; "C25H28F2N4O3"] | 3.30 |
| 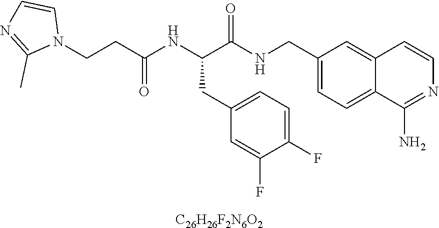 [SMILES: Cc1nccn1CCC(=O)N[C@@H](Cc1ccc(F)c(F)c1)C(=O)NCc1ccc2c(N)nccc2c1 ; "C26H26F2N6O2"] | 3.31 |
| 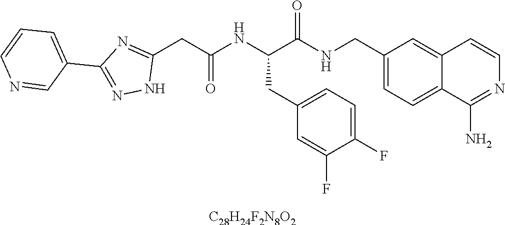 [SMILES: Nc1nccc2cc(CNC(=O)[C@H](Cc3ccc(F)c(F)c3)NC(=O)Cc3nc(-c4cccnc4)n[nH]3)ccc12 ; "C28H24F2N8O2"] | 3.32 |
| 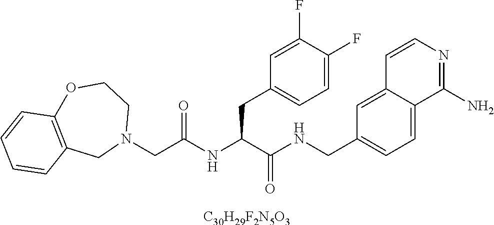 [SMILES: Nc1nccc2cc(CNC(=O)[C@H](Cc3ccc(F)c(F)c3)NC(=O)CN3CCOc4ccccc4C3)ccc12 ; "C30H29F2N5O3"] | 3.33 |
| 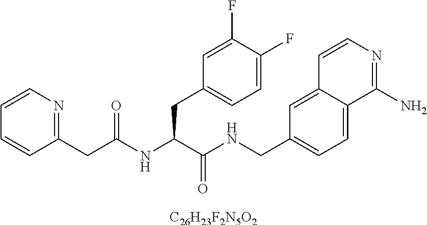 | 3.34 |
| 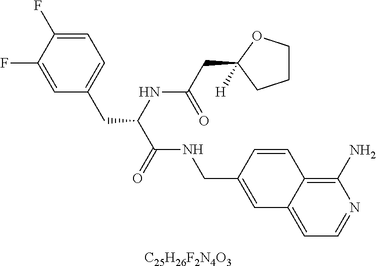 [SMILES: Nc1nccc2cc(CNC(=O)[C@H](Cc3ccc(F)c(F)c3)NC(=O)C[C@@H]3CCCO3)ccc12 ; "C25H26F2N4O3"] | 3.35 |
| 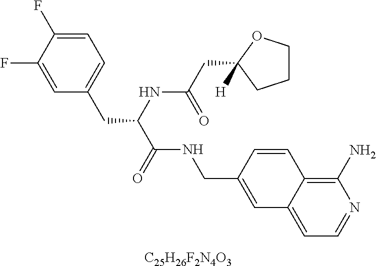 [SMILES: Nc1nccc2cc(CNC(=O)[C@H](Cc3ccc(F)c(F)c3)NC(=O)C[C@H]3CCCO3)ccc12 ; "C25H26F2N4O3"] | 3.36 |
| 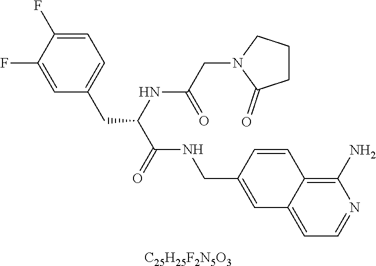 [SMILES: Nc1nccc2cc(CNC(=O)[C@H](Cc3ccc(F)c(F)c3)NC(=O)CN3CCCC3=O)ccc12 ; "C25H25F2N5O3"] | 3.37 |
| 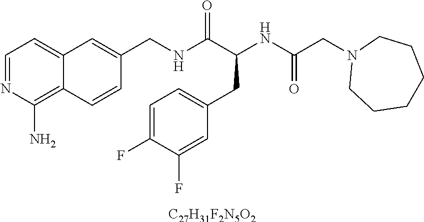 | 3.38 |
| 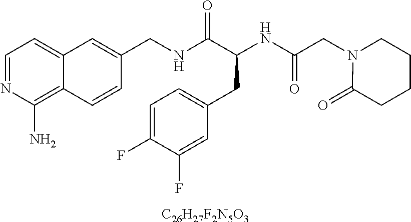 | 3.39 |
| 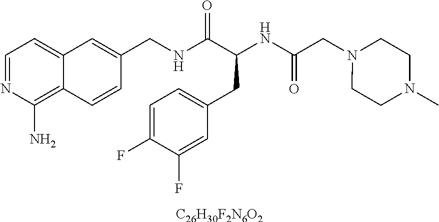 | 3.40 |
| 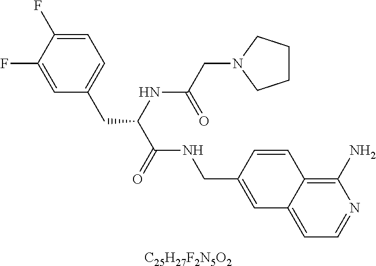 [SMILES: Nc1nccc2cc(CNC(=O)[C@H](Cc3ccc(F)c(F)c3)NC(=O)CN3CCCC3)ccc12 ; "C25H27F2N5O2"] | 3.41 |
| 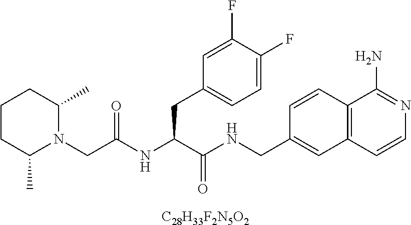 | 3.42 |

TABLE 3

| Structure | Example |
|---|---|
| Molecular formula | No. |
| 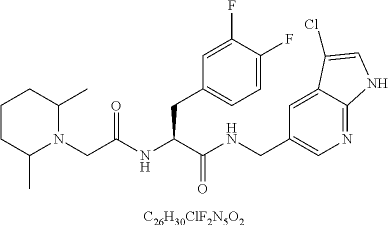 [SMILES: CC1CCCC(C)N1CC(=O)N[C@@H](Cc1ccc(F)c(F)c1)C(=O)NCc1cnc2[nH]cc(Cl)c2c1 ; "C26H30ClF2N5O2"] | 3.101 |
| 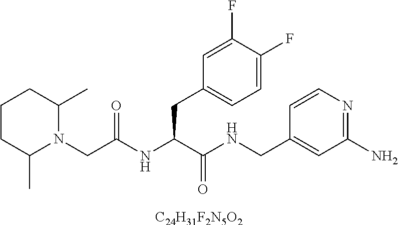 [SMILES: CC1CCCC(C)N1CC(=O)N[C@@H](Cc1ccc(F)c(F)c1)C(=O)NCc1ccnc(N)c1 ; "C24H31F2N5O2"] | 3.102 |
| 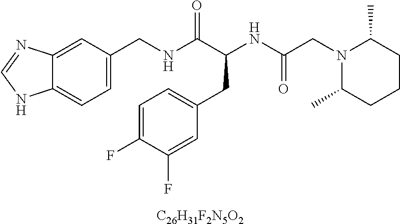 | 3.103 |
| 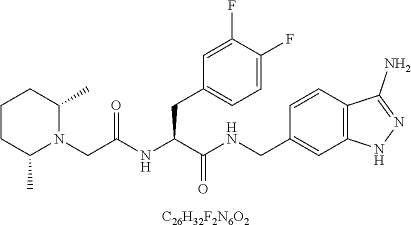 | 3.104 |
| 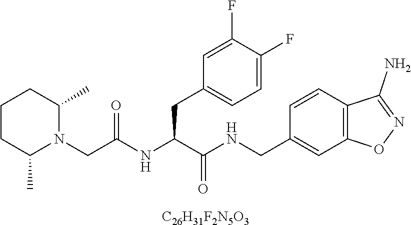 | 3.105 |

TABLE 4

| Structure | Example |
|---|---|
| Molecular formula | No. |
| 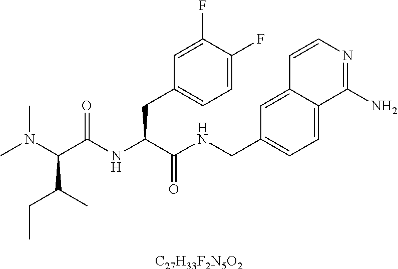 [SMILES: CCC(C)[C@H](C(=O)N[C@@H](Cc1ccc(F)c(F)c1)C(=O)NCc1ccc2c(N)nccc2c1)N(C)C ; "C27H33F2N5O2"] | 5.101 |
| 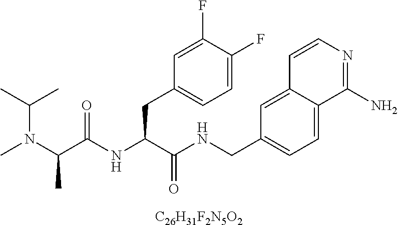 | 5.102 |
| 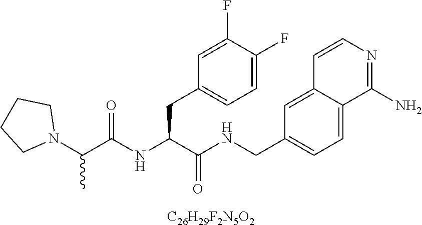 | 5.103 |
| 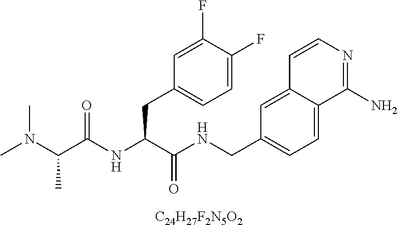 | 5.104 |
| 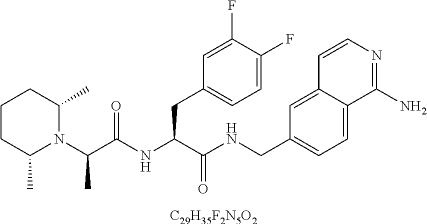 | 5.105 |
| 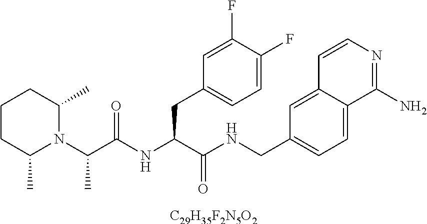 | 5.106 |

TABLE 5

| Structure | Example |
|---|---|
| Molecular formula | No. |
| 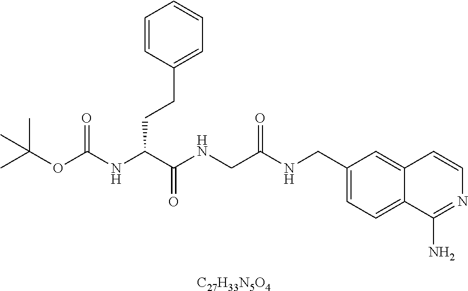 [SMILES: CC(C)(C)OC(=O)N[C@H](CCc1ccccc1)C(=O)NCC(=O)NCc1ccc2c(N)nccc2c1 ; "C27H33N5O4"] | 6.01 |
| 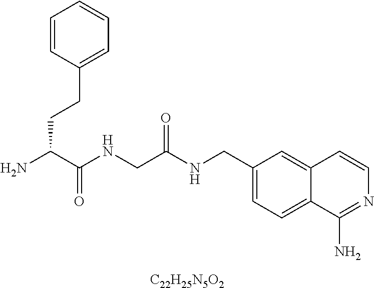 [SMILES: Nc1nccc2cc(CNC(=O)CNC(=O)[C@H](N)CCc3ccccc3)ccc12 ; "C22H25N5O2"] | 6.02 |
| 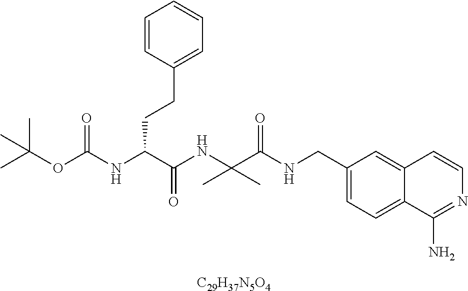 [SMILES: CC(C)(C)OC(=O)N[C@H](CCc1ccccc1)C(=O)NC(C)(C)C(=O)NCc1ccc2c(N)nccc2c1 ; "C29H37N5O4"] | 6.03 |
| 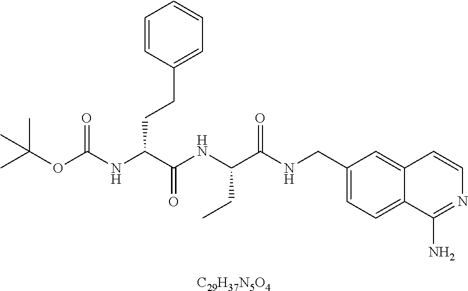 [SMILES: CC[C@H](NC(=O)[C@@H](CCc1ccccc1)NC(=O)OC(C)(C)C)C(=O)NCc1ccc2c(N)nccc2c1 ; "C29H37N5O4"] | 6.04 |
| 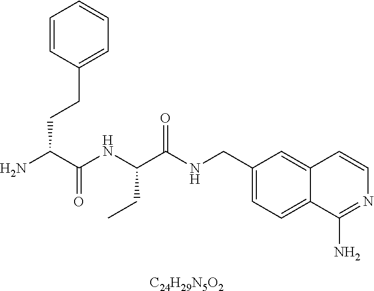 [SMILES: CC[C@H](NC(=O)[C@H](N)CCc1ccccc1)C(=O)NCc1ccc2c(N)nccc2c1 ; "C24H29N5O2"] | 6.05 |
| 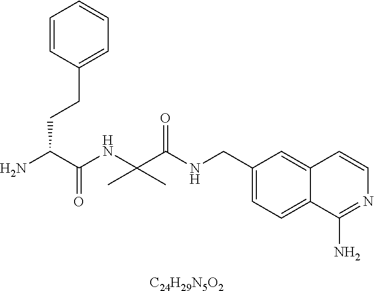 [SMILES: CC(C)(NC(=O)[C@H](N)CCc1ccccc1)C(=O)NCc1ccc2c(N)nccc2c1 ; "C24H29N5O2"] | 6.06 |
| 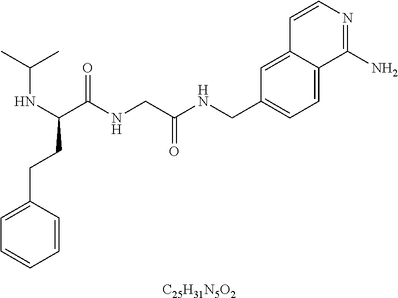 [SMILES: CC(C)N[C@H](CCc1ccccc1)C(=O)NCC(=O)NCc1ccc2c(N)nccc2c1 ; "C25H31N5O2"] | 6.07 |
| 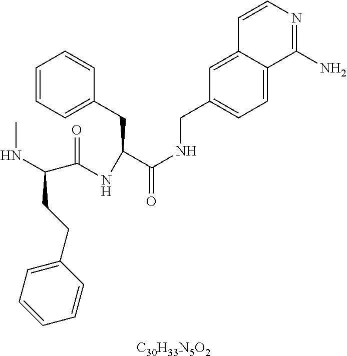 [SMILES: CN[C@H](CCc1ccccc1)C(=O)N[C@@H](Cc1ccccc1)C(=O)NCc1ccc2c(N)nccc2c1 ; "C30H33N5O2"] | 6.08 |
| 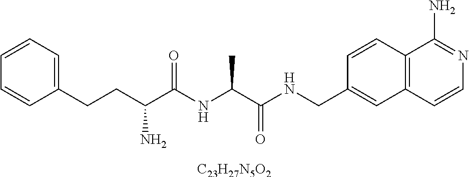 [SMILES: C[C@H](NC(=O)[C@H](N)CCc1ccccc1)C(=O)NCc1ccc2c(N)nccc2c1 ; "C23H27N5O2"] | 6.09 |
| 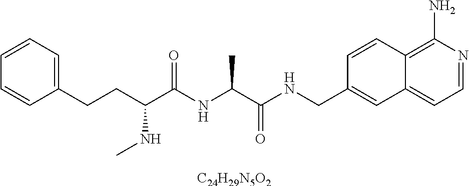 [SMILES: CN[C@H](CCc1ccccc1)C(=O)N[C@@H](C)C(=O)NCc1ccc2c(N)nccc2c1 ; "C24H29N5O2"] | 6.10 |
| 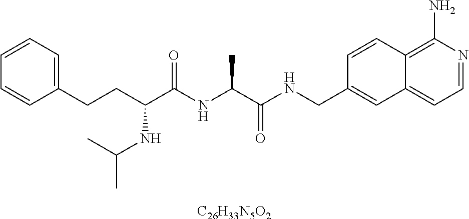 [SMILES: CC(C)N[C@H](CCc1ccccc1)C(=O)N[C@@H](C)C(=O)NCc1ccc2c(N)nccc2c1 ; "C26H33N5O2"] | 6.11 |
| 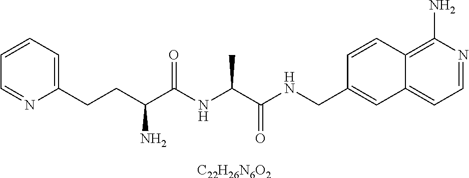 [SMILES: C[C@H](NC(=O)[C@@H](N)CCc1ccccn1)C(=O)NCc1ccc2c(N)nccc2c1 ; "C22H26N6O2"] | 6.12 |
| 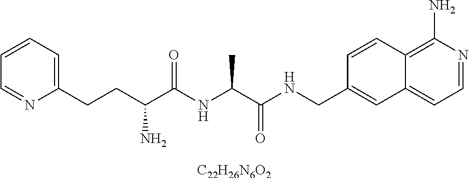 [SMILES: C[C@H](NC(=O)[C@H](N)CCc1ccccn1)C(=O)NCc1ccc2c(N)nccc2c1 ; "C22H26N6O2"] | 6.13 |
| 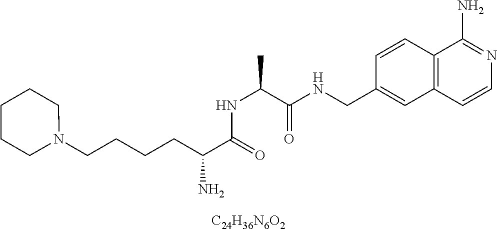 | 6.14 |
| 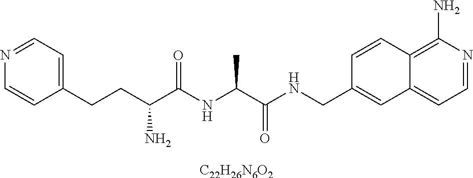 [SMILES: C[C@H](NC(=O)[C@H](N)CCc1ccncc1)C(=O)NCc1ccc2c(N)nccc2c1 ; "C22H26N6O2"] | 6.15 |
| 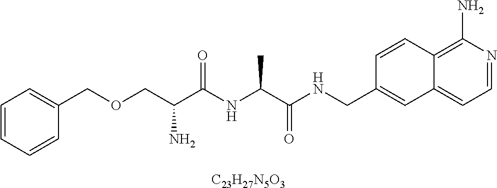 [SMILES: C[C@H](NC(=O)[C@H](N)COCc1ccccc1)C(=O)NCc1ccc2c(N)nccc2c1 ; "C23H27N5O3"] | 6.16 |
| 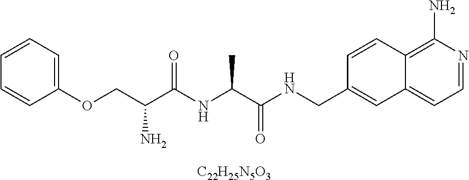 [SMILES: C[C@H](NC(=O)[C@H](N)COc1ccccc1)C(=O)NCc1ccc2c(N)nccc2c1 ; "C22H25N5O3"] | 6.17 |
| 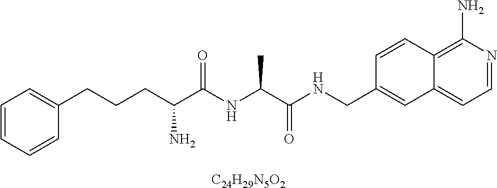 [SMILES: C[C@H](NC(=O)[C@H](N)CCCc1ccccc1)C(=O)NCc1ccc2c(N)nccc2c1 ; "C24H29N5O2"] | 6.18 |
| 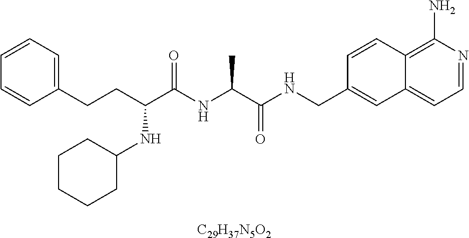 [SMILES: C[C@H](NC(=O)[C@@H](CCc1ccccc1)NC1CCCCC1)C(=O)NCc1ccc2c(N)nccc2c1 ; "C29H37N5O2"] | 6.19 |
| 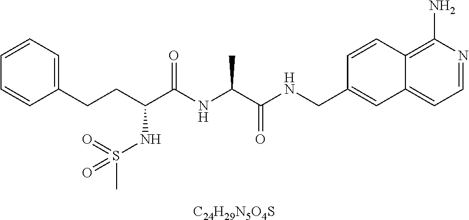 [SMILES: C[C@H](NC(=O)[C@@H](CCc1ccccc1)NS(C)(=O)=O)C(=O)NCc1ccc2c(N)nccc2c1 ; "C24H29N5O4S"] | 6.20 |
| 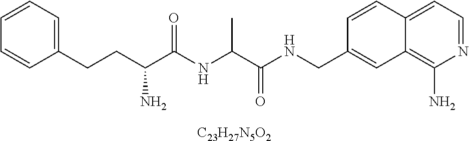 [SMILES: CC(NC(=O)[C@H](N)CCc1ccccc1)C(=O)NCc1ccc2ccnc(N)c2c1 ; "C23H27N5O2"] | 6.21 |
| 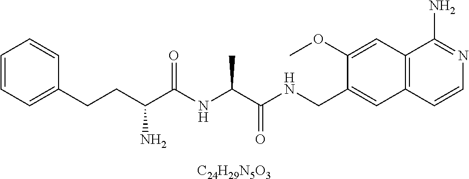 [SMILES: COc1cc2c(N)nccc2cc1CNC(=O)[C@H](C)NC(=O)[C@H](N)CCc1ccccc1 ; "C24H29N5O3"] | 6.22 |
| 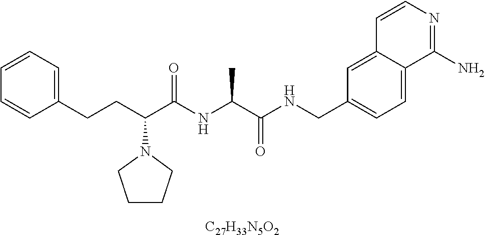 [SMILES: C[C@H](NC(=O)[C@@H](CCc1ccccc1)N1CCCC1)C(=O)NCc1ccc2c(N)nccc2c1 ; "C27H33N5O2"] | 6.23 |
| 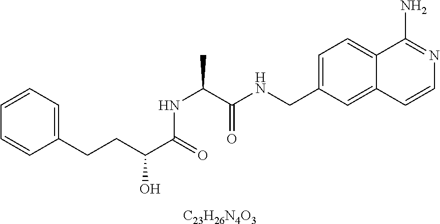 [SMILES: C[C@H](NC(=O)[C@H](O)CCc1ccccc1)C(=O)NCc1ccc2c(N)nccc2c1 ; "C23H26N4O3"] | 6.24 |
| 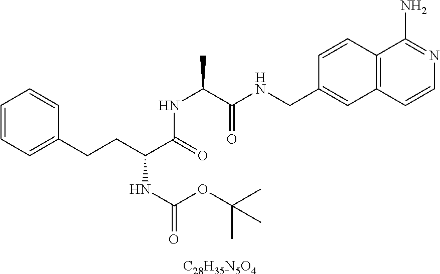 [SMILES: C[C@H](NC(=O)[C@@H](CCc1ccccc1)NC(=O)OC(C)(C)C)C(=O)NCc1ccc2c(N)nccc2c1 ; "C28H35N5O4"] | 6.25 |
| 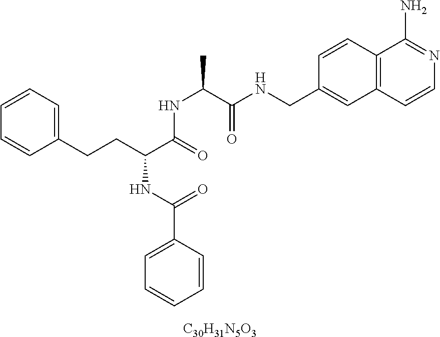 [SMILES: C[C@H](NC(=O)[C@@H](CCc1ccccc1)NC(=O)c1ccccc1)C(=O)NCc1ccc2c(N)nccc2c1 ; "C30H31N5O3"] | 6.26 |

TABLE 6

| Structure | Example |
|---|---|
| Molecular formula | No. |
| 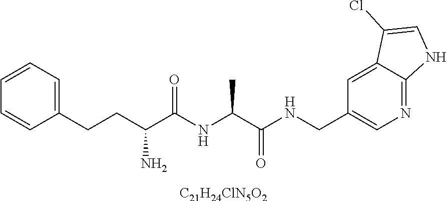 [SMILES: C[C@H](NC(=O)[C@H](N)CCc1ccccc1)C(=O)NCc1cnc2[nH]cc(Cl)c2c1 ; "C21H24ClN5O2"] | 9.01 |
| 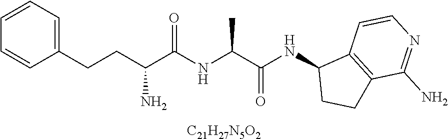 [SMILES: C[C@H](NC(=O)[C@H](N)CCc1ccccc1)C(=O)N[C@@H]1CCc2c1ccnc2N ; "C21H27N5O2"] | 9.02 |
| 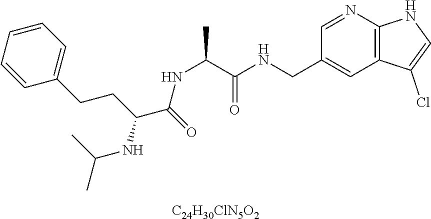 [SMILES: CC(C)N[C@H](CCc1ccccc1)C(=O)N[C@@H](C)C(=O)NCc1cnc2[nH]cc(Cl)c2c1 ; "C24H30ClN5O2"] | 9.03 |
| 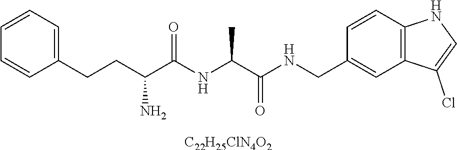 [SMILES: C[C@H](NC(=O)[C@H](N)CCc1ccccc1)C(=O)NCc1ccc2[nH]cc(Cl)c2c1 ; "C22H25ClN4O2"] | 9.04 |
| 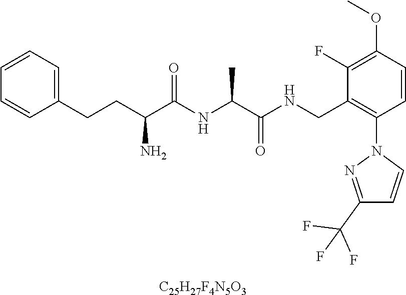 [SMILES: COc1ccc(-n2ccc(C(F)(F)F)n2)c(CNC(=O)[C@H](C)NC(=O)[C@@H](N)CCc2ccccc2)c1F ; "C25H27F4N5O3"] | 9.05 |
| 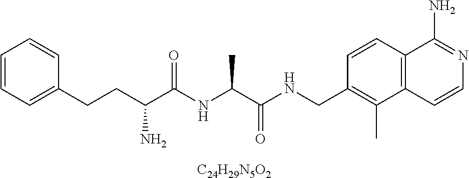 [SMILES: Cc1c(CNC(=O)[C@H](C)NC(=O)[C@H](N)CCc2ccccc2)ccc2c(N)nccc12 ; "C24H29N5O2"] | 9.06 |
| 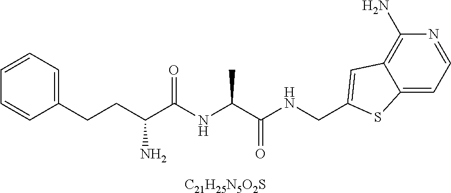 [SMILES: C[C@H](NC(=O)[C@H](N)CCc1ccccc1)C(=O)NCc1cc2c(N)nccc2s1 ; "C21H25N5O2S"] | 9.07 |
| 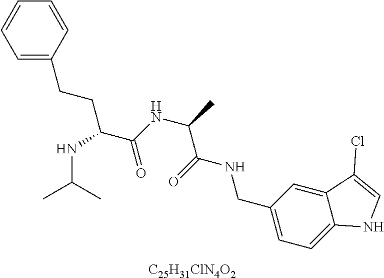 [SMILES: CC(C)N[C@H](CCc1ccccc1)C(=O)N[C@@H](C)C(=O)NCc1ccc2[nH]cc(Cl)c2c1 ; "C25H31ClN4O2"] | 9.08 |
| 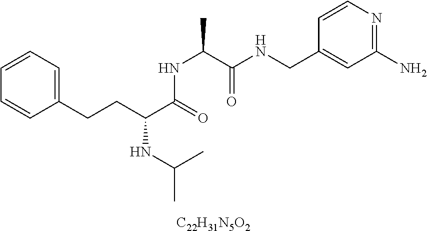 [SMILES: CC(C)N[C@H](CCc1ccccc1)C(=O)N[C@@H](C)C(=O)NCc1ccnc(N)c1 ; "C22H31N5O2"] | 9.09 |
| 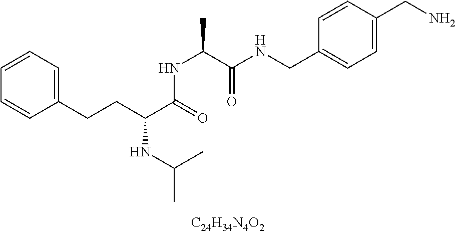 | 9.10 |
| 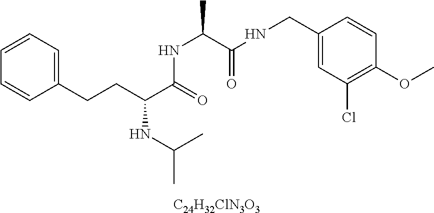 [SMILES: COc1ccc(CNC(=O)[C@H](C)NC(=O)[C@@H](CCc2ccccc2)NC(C)C)cc1Cl ; "C24H32ClN3O3"] | 9.11 |
| 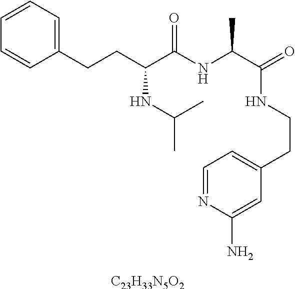 [SMILES: CC(C)N[C@H](CCc1ccccc1)C(=O)N[C@@H](C)C(=O)NCCc1ccnc(N)c1 ; "C23H33N5O2"] | 9.12 |
| 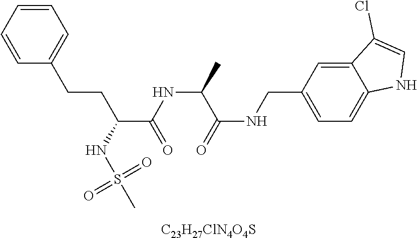 [SMILES: C[C@H](NC(=O)[C@@H](CCc1ccccc1)NS(C)(=O)=O)C(=O)NCc1ccc2[nH]cc(Cl)c2c1 ; "C23H27ClN4O4S"] | 9.16 |
| 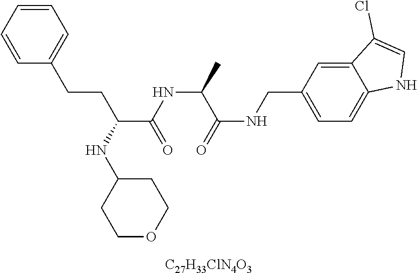 [SMILES: C[C@H](NC(=O)[C@@H](CCc1ccccc1)NC1CCOCC1)C(=O)NCc1ccc2[nH]cc(Cl)c2c1 ; "C27H33ClN4O3"] | 9.17 |
| 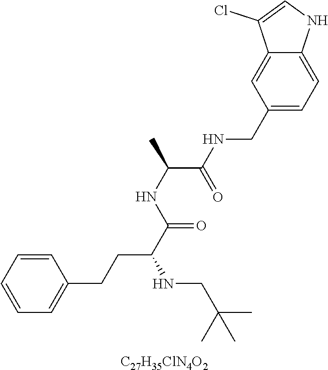 [SMILES: C[C@H](NC(=O)[C@@H](CCc1ccccc1)NCC(C)(C)C)C(=O)NCc1ccc2[nH]cc(Cl)c2c1 ; "C27H35ClN4O2"] | 9.18 |
| 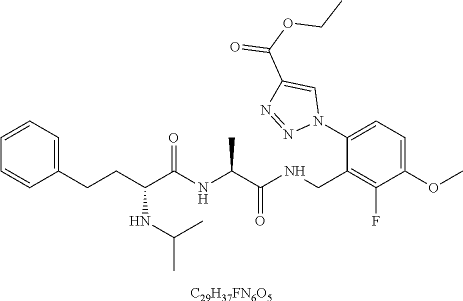 | 9.19 |
| 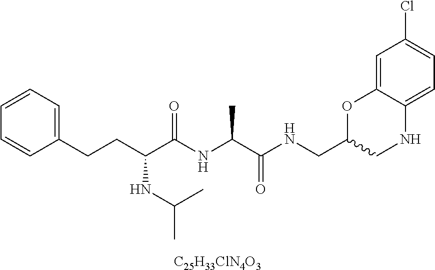 [SMILES: CC(C)N[C@H](CCc1ccccc1)C(=O)N[C@@H](C)C(=O)NCC1CNc2ccc(Cl)cc2O1 ; "C25H33ClN4O3"] | 9.20 |
| 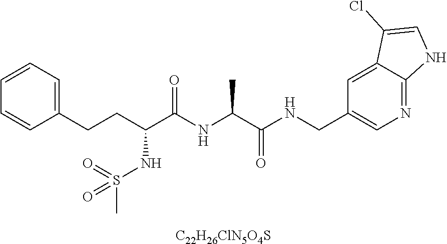 [SMILES: C[C@H](NC(=O)[C@@H](CCc1ccccc1)NS(C)(=O)=O)C(=O)NCc1cnc2[nH]cc(Cl)c2c1 ; "C22H26ClN5O4S"] | 9.21 |
| 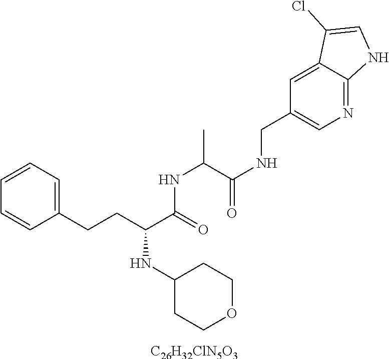 | 9.22 |
| 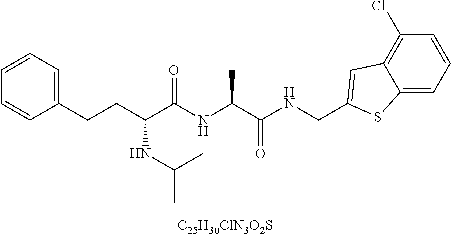 [SMILES: CC(C)N[C@H](CCc1ccccc1)C(=O)N[C@@H](C)C(=O)NCc1cc2c(Cl)cccc2s1 ; "C25H30ClN3O2S"] | 9.23 |
| 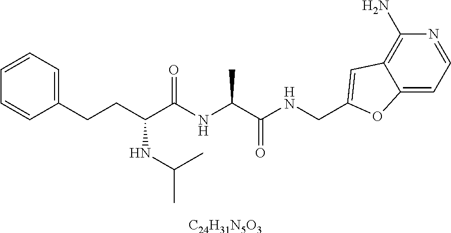 [SMILES: CC(C)N[C@H](CCc1ccccc1)C(=O)N[C@@H](C)C(=O)NCc1cc2c(N)nccc2o1 ; "C24H31N5O3"] | 9.24 |
| 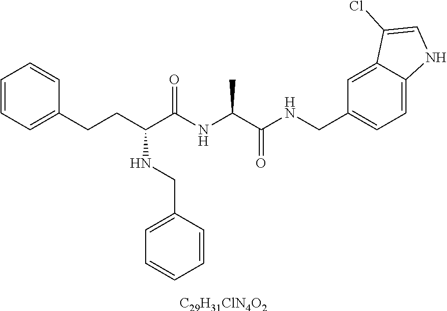 [SMILES: C[C@H](NC(=O)[C@@H](CCc1ccccc1)NCc1ccccc1)C(=O)NCc1ccc2[nH]cc(Cl)c2c1 ; "C29H31ClN4O2"] | 9.25 |
| 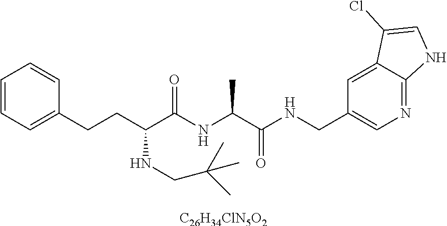 | 9.26 |
| 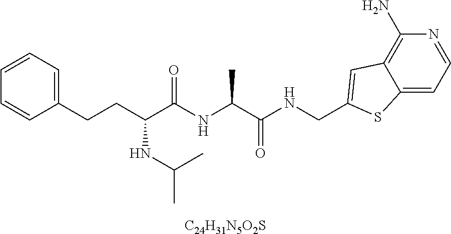 [SMILES: CC(C)N[C@H](CCc1ccccc1)C(=O)N[C@@H](C)C(=O)NCc1cc2c(N)nccc2s1 ; "C24H31N5O2S"] | 9.27 |

TABLE 7

| Structure | Example |
|---|---|
| Molecular formula | No. |
| 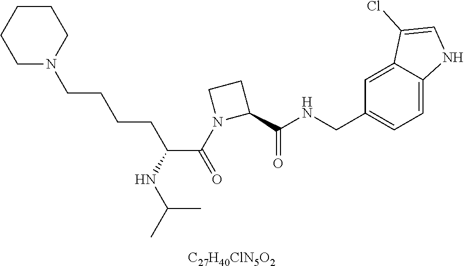 [SMILES: CC(C)N[C@H](CCCCN1CCCCC1)C(=O)N1CC[C@H]1C(=O)NCc1ccc2[nH]cc(Cl)c2c1 ; "C27H40ClN5O2"] | 10.01 |
| 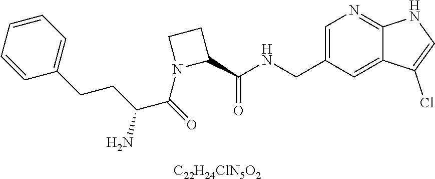 | 10.02 |
| 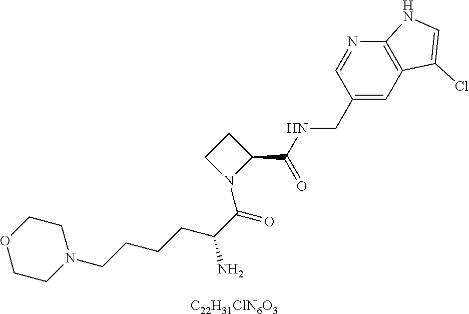 [SMILES: N[C@H](CCCCN1CCOCC1)C(=O)N1CC[C@H]1C(=O)NCc1cnc2[nH]cc(Cl)c2c1 ; "C22H31ClN6O3"] | 10.03 |
| 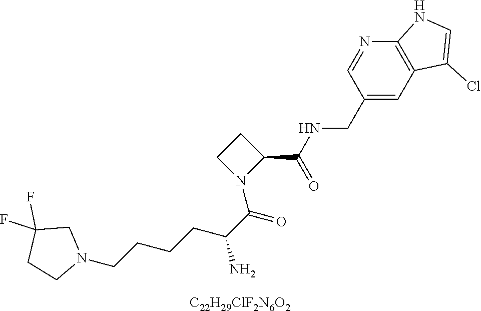 [SMILES: N[C@H](CCCCN1CCC(F)(F)C1)C(=O)N1CC[C@H]1C(=O)NCc1cnc2[nH]cc(Cl)c2c1 ; "C22H29ClF2N6O2"] | 10.04 |
| 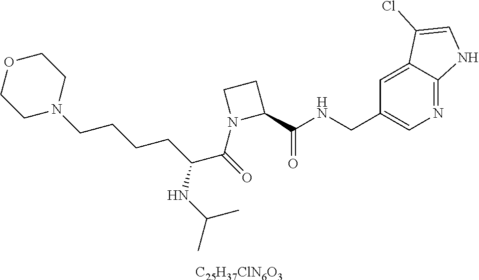 [SMILES: CC(C)N[C@H](CCCCN1CCOCC1)C(=O)N1CC[C@H]1C(=O)NCc1cnc2[nH]cc(Cl)c2c1 ; "C25H37ClN6O3"] | 10.05 |
| 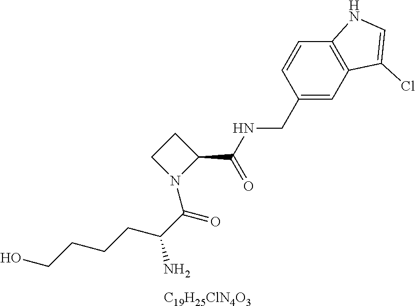 [SMILES: N[C@H](CCCCO)C(=O)N1CC[C@H]1C(=O)NCc1ccc2[nH]cc(Cl)c2c1 ; "C19H25ClN4O3"] | 10.06 |
| 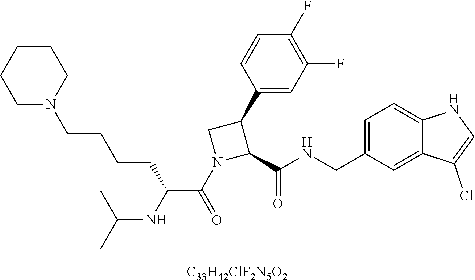 [SMILES: CC(C)N[C@H](CCCCN1CCCCC1)C(=O)N1C[C@@H](c2ccc(F)c(F)c2)[C@H]1C(=O)NCc1ccc2[nH]cc(Cl)c2c1 ; "C33H42ClF2N5O2"] | 10.07 |
| 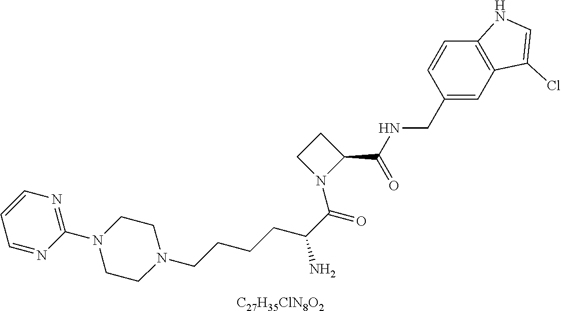 [SMILES: N[C@H](CCCCN1CCN(c2ncccn2)CC1)C(=O)N1CC[C@H]1C(=O)NCc1ccc2[nH]cc(Cl)c2c1 ; "C27H35ClN8O2"] | 10.08 |
| 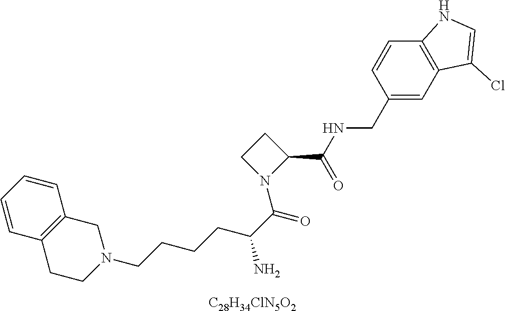 [SMILES: N[C@H](CCCCN1CCc2ccccc2C1)C(=O)N1CC[C@H]1C(=O)NCc1ccc2[nH]cc(Cl)c2c1 ; "C28H34ClN5O2"] | 10.09 |
| 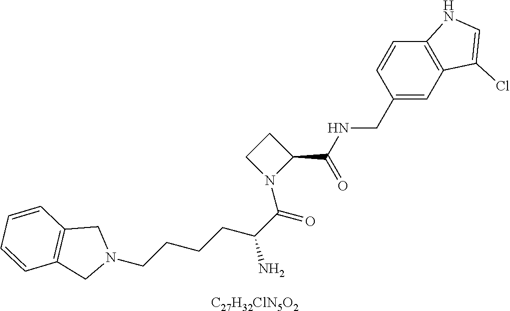 [SMILES: N[C@H](CCCCN1Cc2ccccc2C1)C(=O)N1CC[C@H]1C(=O)NCc1ccc2[nH]cc(Cl)c2c1 ; "C27H32ClN5O2"] | 10.10 |
| 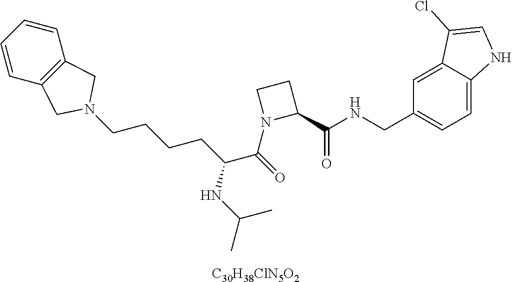 | 10.11 |
| 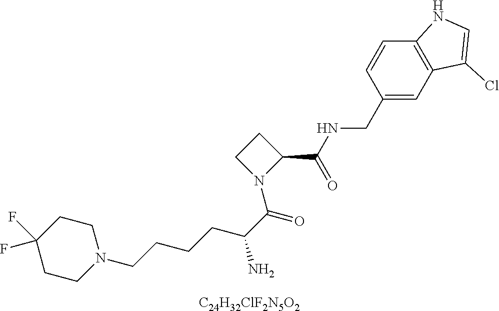 [SMILES: N[C@H](CCCCN1CCC(F)(F)CC1)C(=O)N1CC[C@H]1C(=O)NCc1ccc2[nH]cc(Cl)c2c1 ; "C24H32ClF2N5O2"] | 10.12 |
| 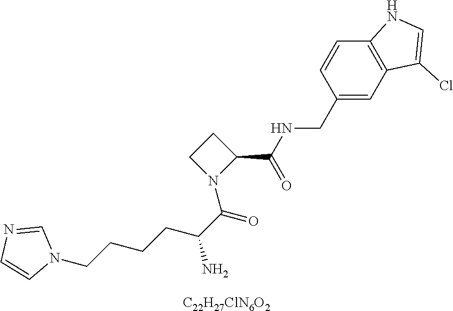 [SMILES: N[C@H](CCCCn1ccnc1)C(=O)N1CC[C@H]1C(=O)NCc1ccc2[nH]cc(Cl)c2c1 ; "C22H27ClN6O2"] | 10.13 |
| 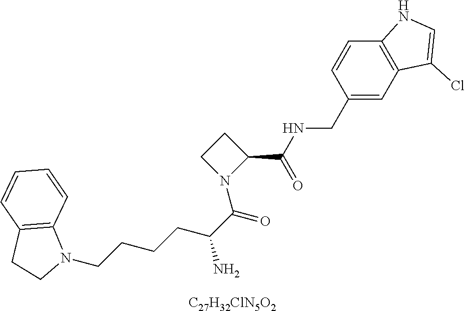 | 10.14 |
| 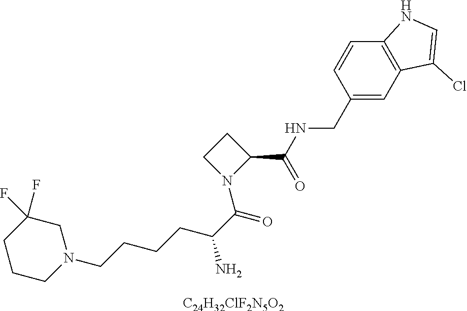 [SMILES: N[C@H](CCCCN1CCCC(F)(F)C1)C(=O)N1CC[C@H]1C(=O)NCc1ccc2[nH]cc(Cl)c2c1 ; "C24H32ClF2N5O2"] | 10.15 |
| 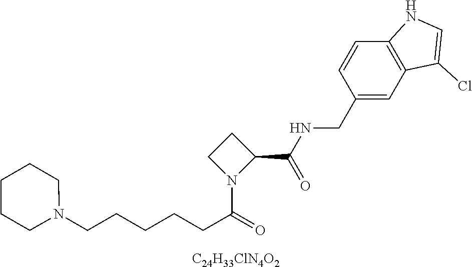 [SMILES: O=C(NCc1ccc2[nH]cc(Cl)c2c1)[C@@H]1CCN1C(=O)CCCCCN1CCCCC1 ; "C24H33ClN4O2"] | 10.16 |
| 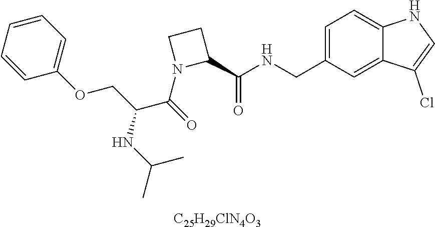 | 10.17 |
| 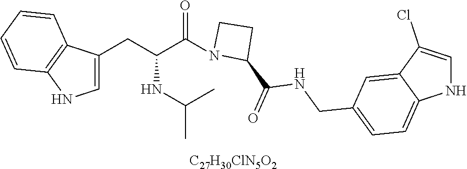 | 10.18 |
| 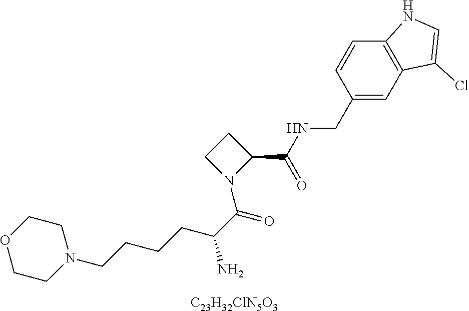 | 10.19 |
| 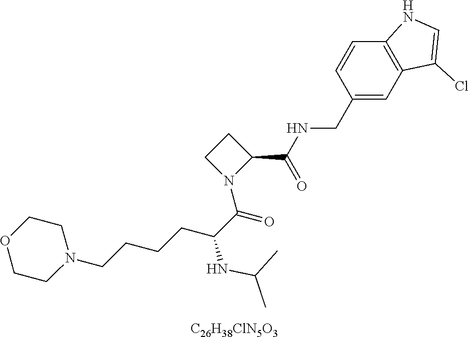 [SMILES: CC(C)N[C@H](CCCCN1CCOCC1)C(=O)N1CC[C@H]1C(=O)NCc1ccc2[nH]cc(Cl)c2c1 ; "C26H38ClN5O3"] | 10.20 |

TABLE 8

| Structure | Example |
|---|---|
| Molecular formula | No. |
| 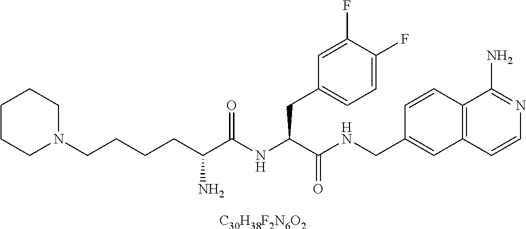 | 12.01 |
| 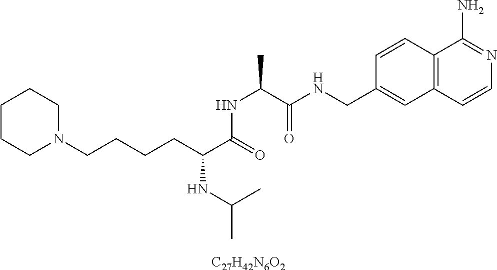 | 12.02 |
| 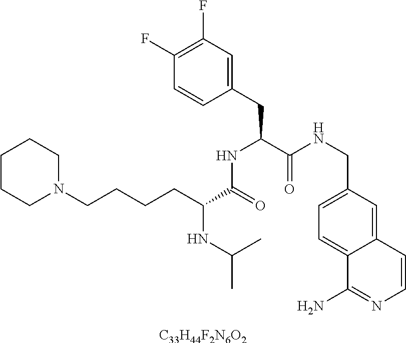 [SMILES: CC(C)N[C@H](CCCCN1CCCCC1)C(=O)N[C@@H](Cc1ccc(F)c(F)c1)C(=O)NCc1ccc2c(N)nccc2c1 ; "C33H44F2N6O2"] | 12.03 |
| 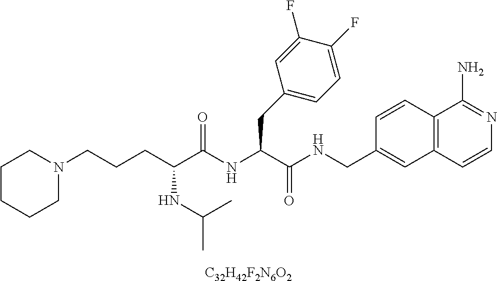 | 12.04 |
| 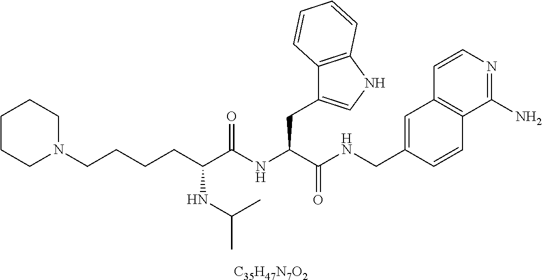 [SMILES: CC(C)N[C@H](CCCCN1CCCCC1)C(=O)N[C@@H](Cc1c[nH]c2ccccc12)C(=O)NCc1ccc2c(N)nccc2c1 ; "C35H47N7O2"] | 12.05 |
| 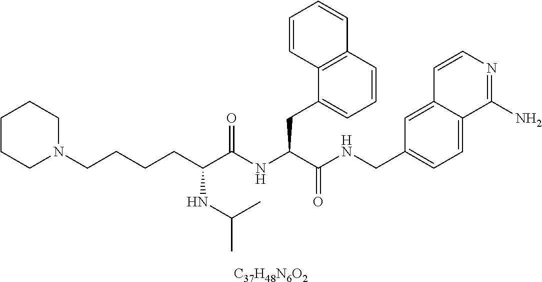 | 12.06 |
| 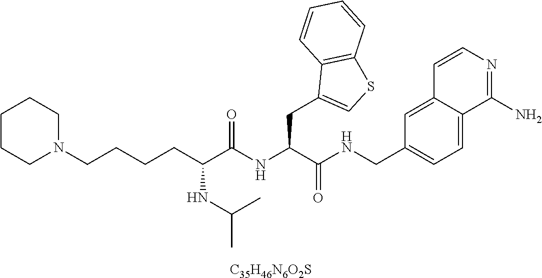 | 12.07 |
| 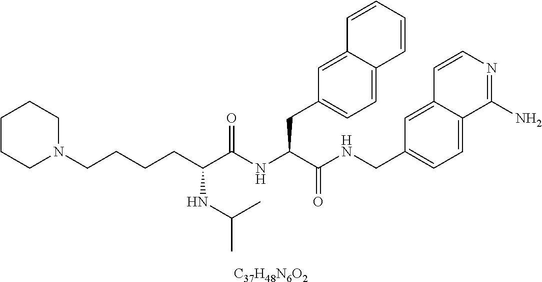 | 12.08 |
| 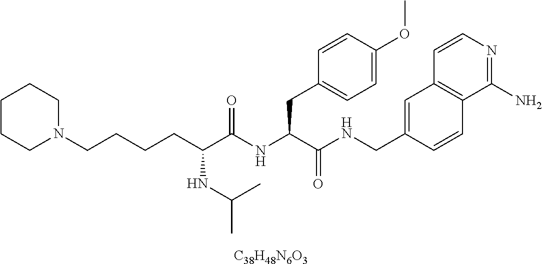 | 12.09 |
| 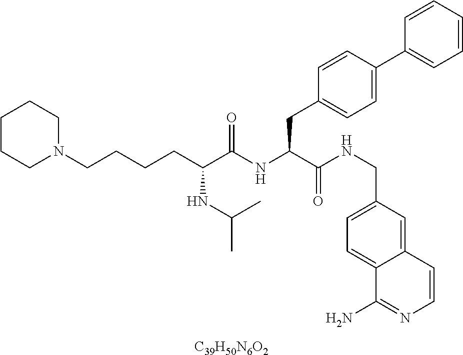 [SMILES: CC(C)N[C@H](CCCCN1CCCCC1)C(=O)N[C@@H](Cc1ccc(-c2ccccc2)cc1)C(=O)NCc1ccc2c(N)nccc2c1 ; "C39H50N6O2"] | 12.10 |
| 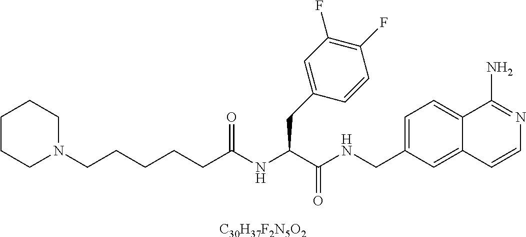 [SMILES: Nc1nccc2cc(CNC(=O)[C@H](Cc3ccc(F)c(F)c3)NC(=O)CCCCCN3CCCCC3)ccc12 ; "C30H37F2N5O2"] | 12.11 |
| 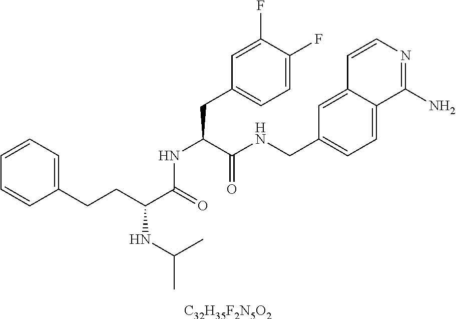 | 12.12 |

TABLE 9

| Structure | Example |
|---|---|
| Molecular formula | No. |
| 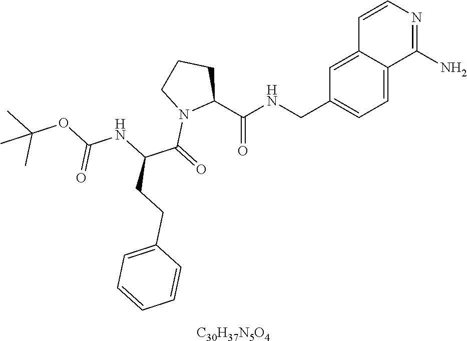 [SMILES: CC(C)(C)OC(=O)N[C@H](CCc1ccccc1)C(=O)N1CCC[C@H]1C(=O)NCc1ccc2c(N)nccc2c1 ; "C30H37N5O4"] | 17.01 |
| 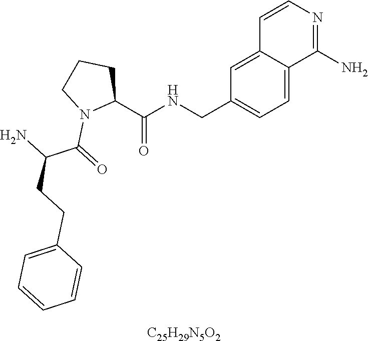 [SMILES: Nc1nccc2cc(CNC(=O)[C@@H]3CCCN3C(=O)[C@H](N)CCc3ccccc3)ccc12 ; "C25H29N5O2"] | 17.02 |
| 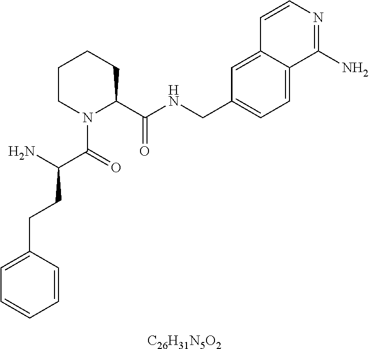 [SMILES: Nc1nccc2cc(CNC(=O)[C@@H]3CCCCN3C(=O)[C@H](N)CCc3ccccc3)ccc12 ; "C26H31N5O2"] | 17.03 |
| 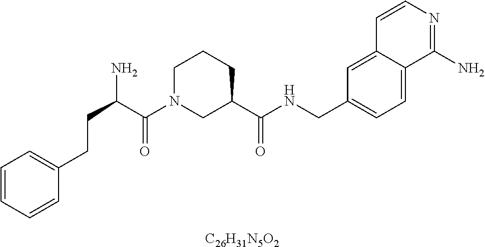 [SMILES: Nc1nccc2cc(CNC(=O)[C@@H]3CCCN(C(=O)[C@H](N)CCc4ccccc4)C3)ccc12 ; "C26H31N5O2"] | 17.04 |
| 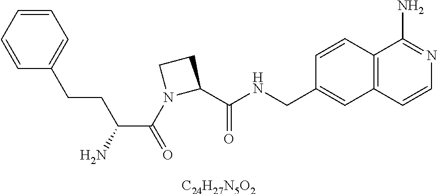 [SMILES: Nc1nccc2cc(CNC(=O)[C@@H]3CCN3C(=O)[C@H](N)CCc3ccccc3)ccc12 ; "C24H27N5O2"] | 17.05 |
| 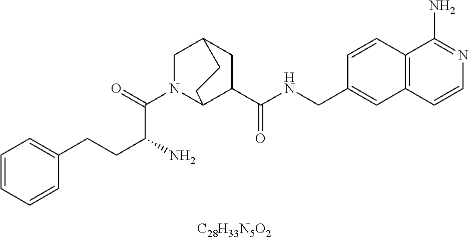 [SMILES: Nc1nccc2cc(CNC(=O)C3CC4CCC3N(C(=O)[C@H](N)CCc3ccccc3)C4)ccc12 ; "C28H33N5O2"] | 17.06 |
| 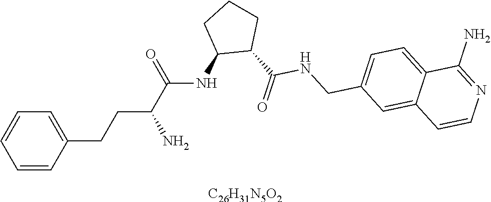 [SMILES: Nc1nccc2cc(CNC(=O)[C@H]3CCC[C@@H]3NC(=O)[C@H](N)CCc3ccccc3)ccc12 ; "C26H31N5O2"] | 17.07 |
| 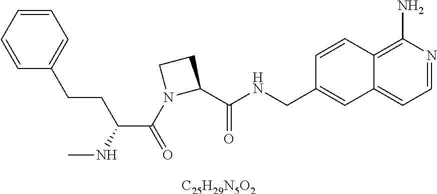 [SMILES: CN[C@H](CCc1ccccc1)C(=O)N1CC[C@H]1C(=O)NCc1ccc2c(N)nccc2c1 ; "C25H29N5O2"] | 17.08 |
| 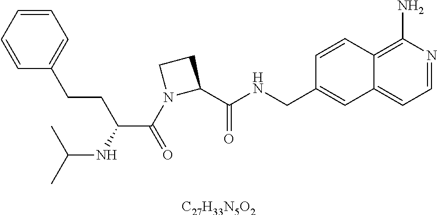 [SMILES: CC(C)N[C@H](CCc1ccccc1)C(=O)N1CC[C@H]1C(=O)NCc1ccc2c(N)nccc2c1 ; "C27H33N5O2"] | 17.09 |
| 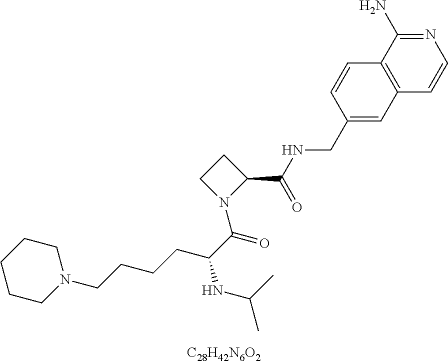 [SMILES: CC(C)N[C@H](CCCCN1CCCCC1)C(=O)N1CC[C@H]1C(=O)NCc1ccc2c(N)nccc2c1 ; "C28H42N6O2"] | 17.10 |
| 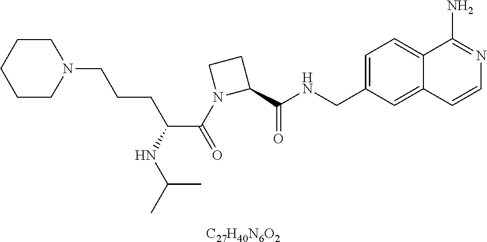 [SMILES: CC(C)N[C@H](CCCN1CCCCC1)C(=O)N1CC[C@H]1C(=O)NCc1ccc2c(N)nccc2c1 ; "C27H40N6O2"] | 17.11 |

TABLE 10

| Structure | Example |
|---|---|
| Molecular formula | No. |
| 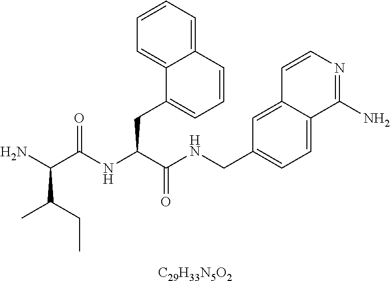 [SMILES: CCC(C)[C@@H](N)C(=O)N[C@@H](Cc1cccc2ccccc12)C(=O)NCc1ccc2c(N)nccc2c1 ; "C29H33N5O2"] | 18.101 |
| 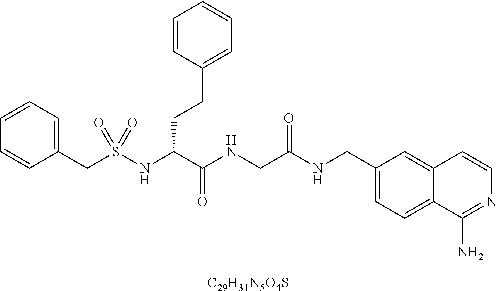 [SMILES: Nc1nccc2cc(CNC(=O)CNC(=O)[C@@H](CCc3ccccc3)NS(=O)(=O)Cc3ccccc3)ccc12 ; "C29H31N5O4S"] | 18.102 |
| 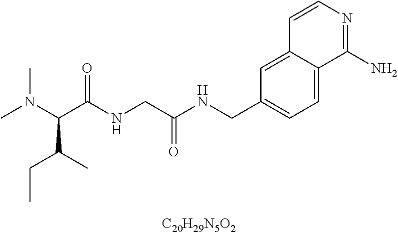 [SMILES: CCC(C)[C@H](C(=O)NCC(=O)NCc1ccc2c(N)nccc2c1)N(C)C ; "C20H29N5O2"] | 18.103 |
| 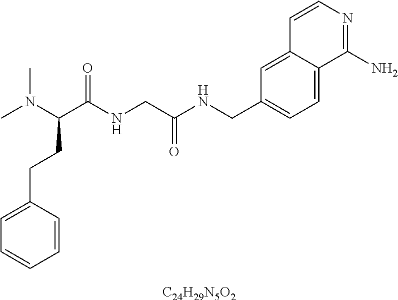 [SMILES: CN(C)[C@H](CCc1ccccc1)C(=O)NCC(=O)NCc1ccc2c(N)nccc2c1 ; "C24H29N5O2"] | 18.104 |
| 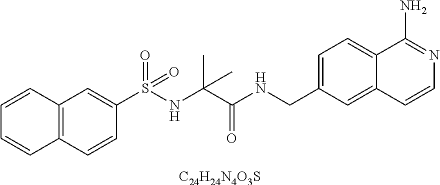 [SMILES: CC(C)(NS(=O)(=O)c1ccc2ccccc2c1)C(=O)NCc1ccc2c(N)nccc2c1 ; "C24H24N4O3S"] | 18.105 |
| 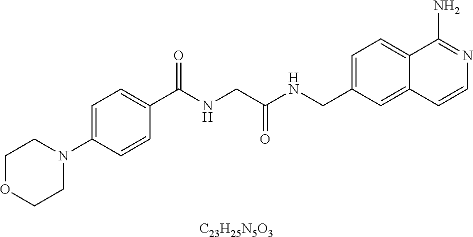 [SMILES: Nc1nccc2cc(CNC(=O)CNC(=O)c3ccc(N4CCOCC4)cc3)ccc12 ; "C23H25N5O3"] | 18.106 |
| 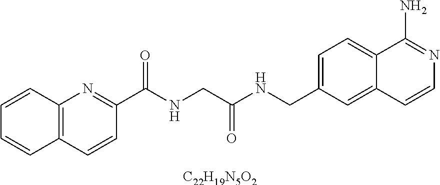 [SMILES: Nc1nccc2cc(CNC(=O)CNC(=O)c3ccc4ccccc4n3)ccc12 ; "C22H19N5O2"] | 18.107 |
| 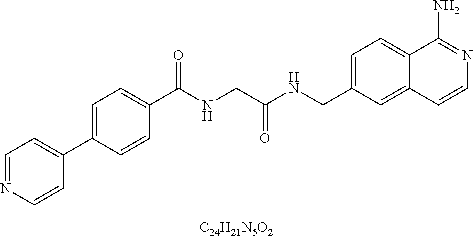 [SMILES: Nc1nccc2cc(CNC(=O)CNC(=O)c3ccc(-c4ccncc4)cc3)ccc12 ; "C24H21N5O2"] | 18.108 |
| 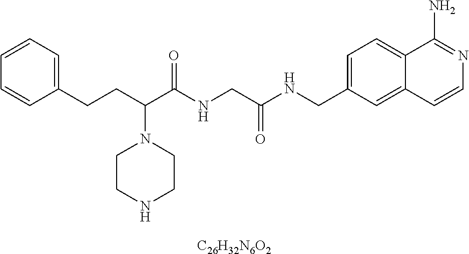 [SMILES: Nc1nccc2cc(CNC(=O)CNC(=O)C(CCc3ccccc3)N3CCNCC3)ccc12 ; "C26H32N6O2"] | 18.109 |

TABLE 11

| Structure | Example |
|---|---|
| Molecular formula | No. |
| 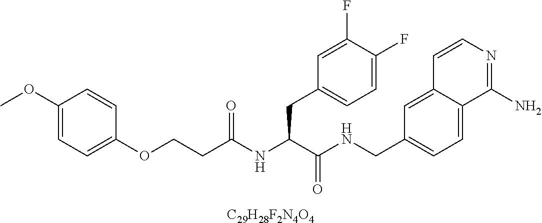 [SMILES: COc1ccc(OCCC(=O)N[C@@H](Cc2ccc(F)c(F)c2)C(=O)NCc2ccc3c(N)nccc3c2)cc1 ; "C29H28F2N4O4"] | 20.01 |
| 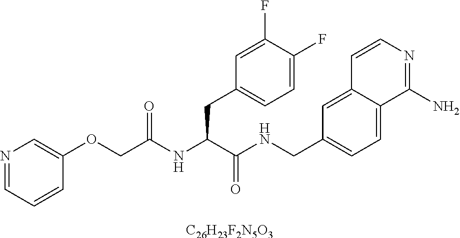 [SMILES: Nc1nccc2cc(CNC(=O)[C@H](Cc3ccc(F)c(F)c3)NC(=O)COc3cccnc3)ccc12 ; "C26H23F2N5O3"] | 20.02 |
| 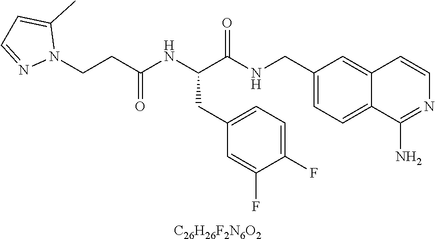 [SMILES: Cc1ccnn1CCC(=O)N[C@@H](Cc1ccc(F)c(F)c1)C(=O)NCc1ccc2c(N)nccc2c1 ; "C26H26F2N6O2"] | 20.03 |
| 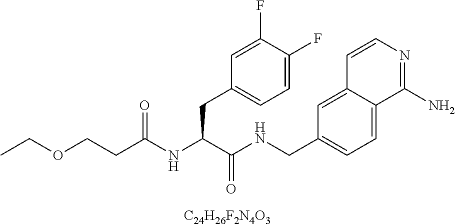 [SMILES: CCOCCC(=O)N[C@@H](Cc1ccc(F)c(F)c1)C(=O)NCc1ccc2c(N)nccc2c1 ; "C24H26F2N4O3"] | 20.04 |
| 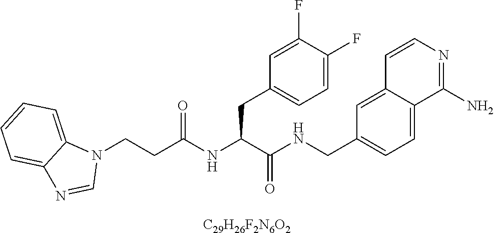 [SMILES: Nc1nccc2cc(CNC(=O)[C@H](Cc3ccc(F)c(F)c3)NC(=O)CCn3cnc4ccccc43)ccc12 ; "C29H26F2N6O2"] | 20.05 |
| 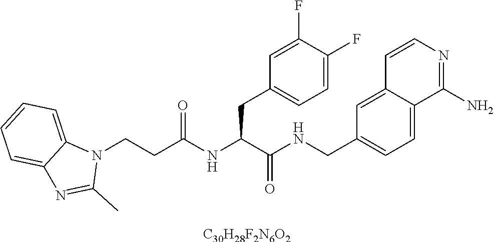 [SMILES: Cc1nc2ccccc2n1CCC(=O)N[C@@H](Cc1ccc(F)c(F)c1)C(=O)NCc1ccc2c(N)nccc2c1 ; "C30H28F2N6O2"] | 20.06 |
| 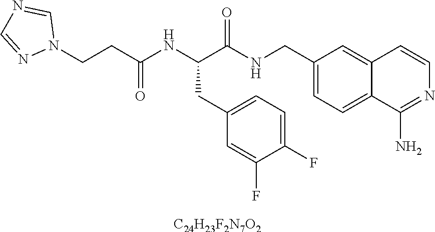 [SMILES: Nc1nccc2cc(CNC(=O)[C@H](Cc3ccc(F)c(F)c3)NC(=O)CCn3cncn3)ccc12 ; "C24H23F2N7O2"] | 20.07 |
| 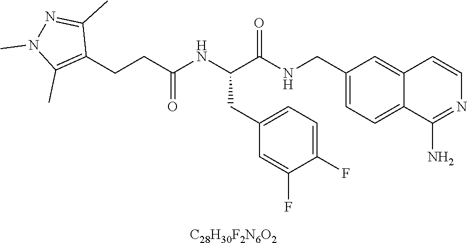 [SMILES: Cc1nn(C)c(C)c1CCC(=O)N[C@@H](Cc1ccc(F)c(F)c1)C(=O)NCc1ccc2c(N)nccc2c1 ; "C28H30F2N6O2"] | 20.08 |
| 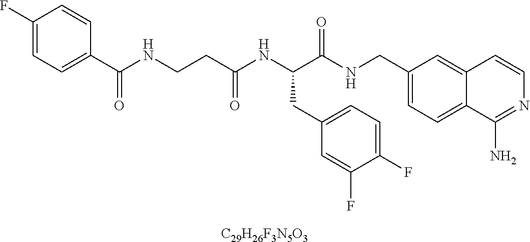 [SMILES: Nc1nccc2cc(CNC(=O)[C@H](Cc3ccc(F)c(F)c3)NC(=O)CCNC(=O)c3ccc(F)cc3)ccc12 ; "C29H26F3N5O3"] | 20.09 |
| 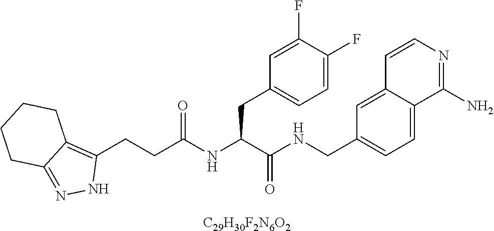 [SMILES: Nc1nccc2cc(CNC(=O)[C@H](Cc3ccc(F)c(F)c3)NC(=O)CCc3[nH]nc4c3CCCC4)ccc12 ; "C29H30F2N6O2"] | 20.10 |

TABLE 12

| Structure | Example |
|---|---|
| Molecular formula | No. |
| 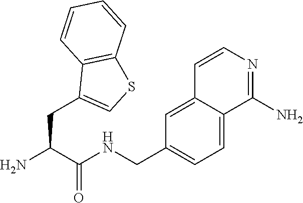 [SMILES: Nc1nccc2cc(CNC(=O)[C@@H](N)Cc3csc4ccccc34)ccc12] | 24.01 |
| C₂₁H₂₀N₄OS | |
| 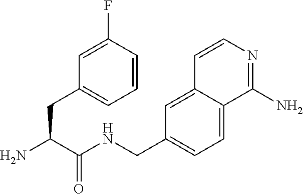 [SMILES: Nc1nccc2cc(CNC(=O)[C@@H](N)Cc3cccc(F)c3)ccc12] | 24.02 |
| C₁₉H₁₉FN₄O | |
| 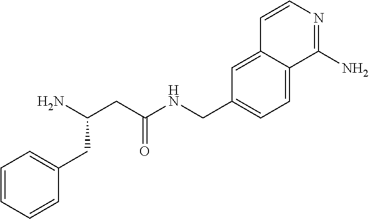 [SMILES: Nc1nccc2cc(CNC(=O)C[C@@H](N)Cc3ccccc3)ccc12] | 24.03 |
| C₂₀H₂₂N₄O | |

TABLE 13

| Structure | Example |
|---|---|
| Molecular formula | No. |
| 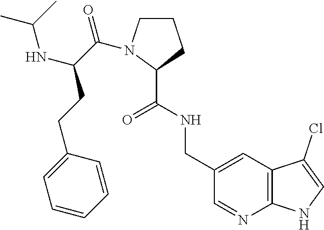 [SMILES: CC(C)N[C@H](CCc1ccccc1)C(=O)N1CCC[C@H]1C(=O)NCc1cnc2[nH]cc(Cl)c2c1] | 27.01 |
| C₂₆H₃₂ClN₅O₂ | |
| 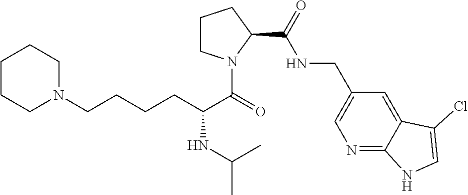 [SMILES: CC(C)N[C@H](CCCCN1CCCCC1)C(=O)N1CCC[C@H]1C(=O)NCc1cnc2[nH]cc(Cl)c2c1] | 27.02 |
| C₂₇H₄₁ClN₆O₂ | |
| 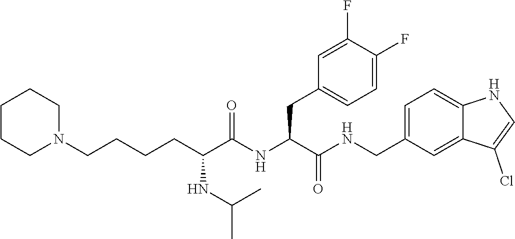 [SMILES: CC(C)N[C@H](CCCCN1CCCCC1)C(=O)N[C@@H](Cc1ccc(F)c(F)c1)C(=O)NCc1ccc2[nH]cc(Cl)c2c1] | 27.03 |
| C₃₂H₄₂ClF₂N₅O₂ | |
| 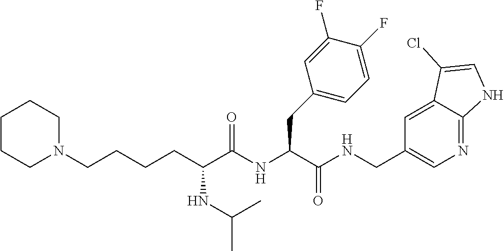 [SMILES: CC(C)N[C@H](CCCCN1CCCCC1)C(=O)N[C@@H](Cc1ccc(F)c(F)c1)C(=O)NCc1cnc2[nH]cc(Cl)c2c1] | 27.04 |
| C₃₁H₄₁ClF₂N₆O₂ | |
| 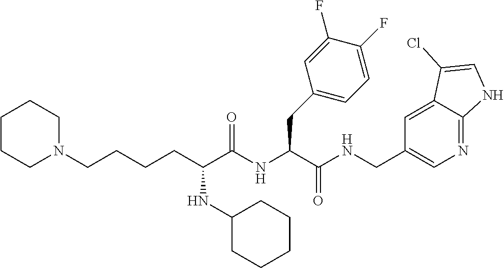 [SMILES: O=C(NCc1cnc2[nH]cc(Cl)c2c1)[C@H](Cc1ccc(F)c(F)c1)NC(=O)[C@@H](CCCCN1CCCCC1)NC1CCCCC1] | 27.05 |
| C₃₄H₄₅ClF₂N₆O₂ | |
| 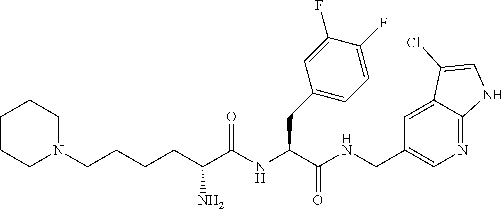 [SMILES: N[C@H](CCCCN1CCCCC1)C(=O)N[C@@H](Cc1ccc(F)c(F)c1)C(=O)NCc1cnc2[nH]cc(Cl)c2c1] | 27.06 |
| C₂₈H₃₅ClF₂N₆O₂ | |
| 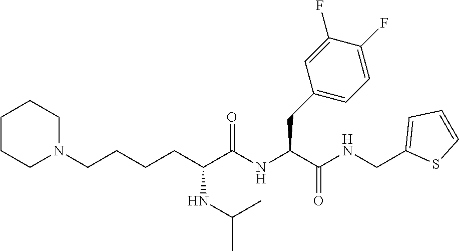 [SMILES: CC(C)N[C@H](CCCCN1CCCCC1)C(=O)N[C@@H](Cc1ccc(F)c(F)c1)C(=O)NCc1cccs1] | 27.07 |
| C₂₈H₄₀F₂N₄O₂S | |
| 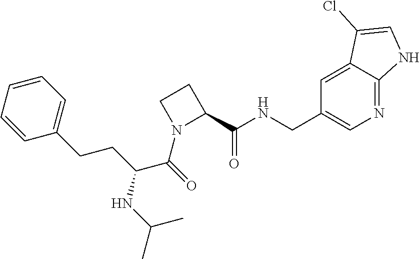 [SMILES: CC(C)N[C@H](CCc1ccccc1)C(=O)N1CC[C@H]1C(=O)NCc1cnc2[nH]cc(Cl)c2c1] | 27.08 |
| C₂₅H₃₀ClN₅O₂ | |

TABLE 14

| Structure | Example |
|---|---|
| Molecular formula | No. |
| 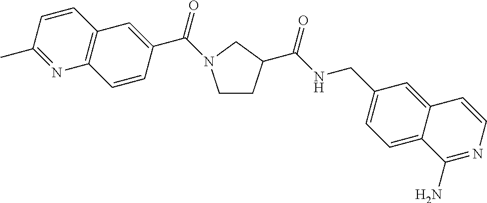 [SMILES: Cc1ccc2cc(C(=O)N3CCC(C(=O)NCc4ccc5c(N)nccc5c4)C3)ccc2n1] | 32.01 |
| C₂₆H₂₅N₅O₂ | |
| 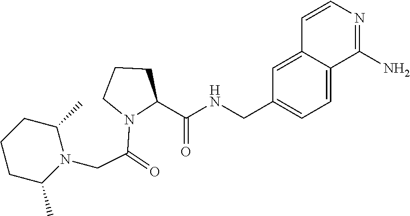 [SMILES: C[C@@H]1CCC[C@H](C)N1CC(=O)N1CCC[C@H]1C(=O)NCc1ccc2c(N)nccc2c1] | 32.02 |
| C₂₄H₃₃N₅O₂ | |
| 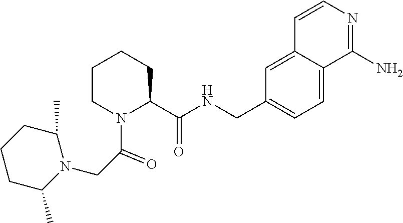 [SMILES: C[C@@H]1CCC[C@H](C)N1CC(=O)N1CCCC[C@H]1C(=O)NCc1ccc2c(N)nccc2c1] | 32.03 |
| C₂₅H₃₅N₅O₂ | |
| 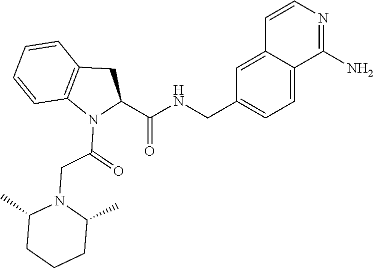 [SMILES: C[C@@H]1CCC[C@H](C)N1CC(=O)N1c2ccccc2C[C@H]1C(=O)NCc1ccc2c(N)nccc2c1] | 32.04 |
| C₂₈H₃₃N₅O₂ | |
| 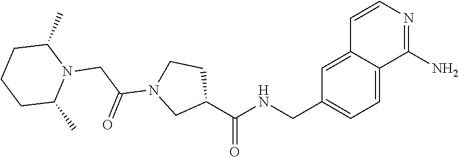 [SMILES: C[C@@H]1CCC[C@H](C)N1CC(=O)N1CC[C@H](C(=O)NCc2ccc3c(N)nccc3c2)C1] | 32.06 |
| C₂₄H₃₃N₅O₂ | |
| 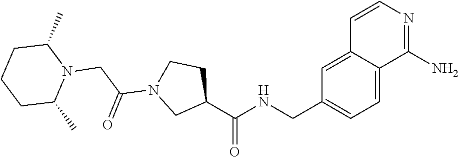 [SMILES: C[C@@H]1CCC[C@H](C)N1CC(=O)N1CC[C@@H](C(=O)NCc2ccc3c(N)nccc3c2)C1] | 32.07 |
| C₂₄H₃₃N₅O₂ | |
| 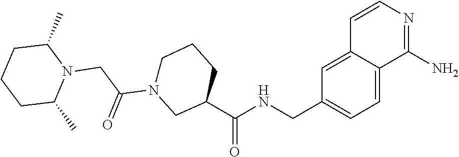 [SMILES: C[C@@H]1CCC[C@H](C)N1CC(=O)N1CCC[C@@H](C(=O)NCc2ccc3c(N)nccc3c2)C1] | 32.08 |
| C₂₅H₃₅N₅O₂ | |
| 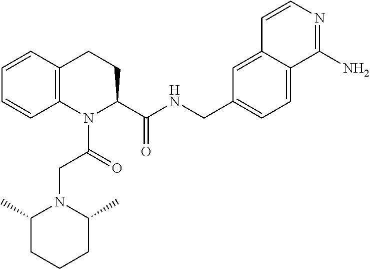 [SMILES: C[C@@H]1CCC[C@H](C)N1CC(=O)N1c2ccccc2CC[C@H]1C(=O)NCc1ccc2c(N)nccc2c1] | 32.09 |
| C₂₆H₃₅N₅O₂ | |
| 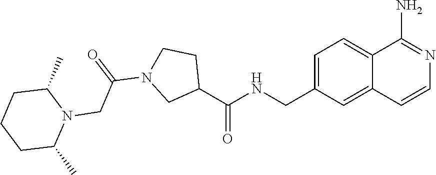 [SMILES: C[C@@H]1CCC[C@H](C)N1CC(=O)N1CCC(C(=O)NCc2ccc3c(N)nccc3c2)C1] | 32.10 |
| C₂₄H₃₃N₅O₂ | |
| 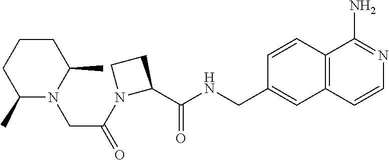 [SMILES: C[C@@H]1CCC[C@H](C)N1CC(=O)N1CC[C@H]1C(=O)NCc1ccc2c(N)nccc2c1] | 32.11 |
| C₂₃H₃₁N₅O₂ | |
| 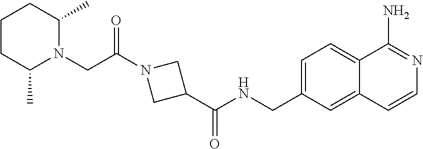 [SMILES: C[C@@H]1CCC[C@H](C)N1CC(=O)N1CC(C(=O)NCc2ccc3c(N)nccc3c2)C1] | 32.12 |
| C₂₃H₃₁N₅O₂ | |
| 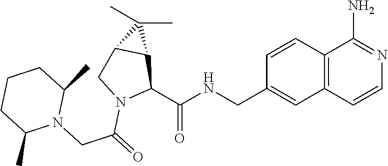 [SMILES: C[C@@H]1CCC[C@H](C)N1CC(=O)N1C[C@H]2[C@@H]([C@H]1C(=O)NCc1ccc3c(N)nccc3c1)C2(C)C] | 32.13 |
| C₂₇H₃₇N₅O₂ | |
| 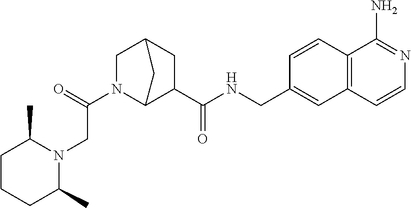 [SMILES: C[C@@H]1CCC[C@H](C)N1CC(=O)N1CC2CC(C(=O)NCc3ccc4c(N)nccc4c3)C1C2] | 32.14 |
| C₂₆H₃₅N₅O₂ | |
| 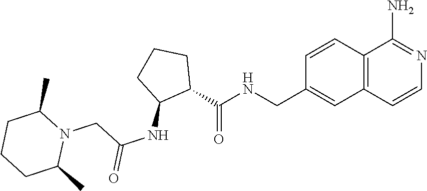 [SMILES: C[C@@H]1CCC[C@H](C)N1CC(=O)N[C@H]1CCC[C@@H]1C(=O)NCc1ccc2c(N)nccc2c1] | 32.15 |
| C₂₅H₃₅N₅O₂ | |

TABLE 15

| | Ex- |
|---|---|
| Structure | ample |
| Molecular formula | No. |
| 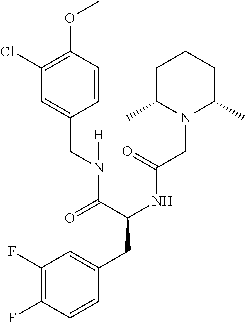 [SMILES: COc1ccc(CNC(=O)[C@H](Cc2ccc(F)c(F)c2)NC(=O)CN2[C@H](C)CCC[C@@H]2C)cc1Cl] | 34.01 |
| C₂₆H₃₂ClF₂N₃O₃ | |
| 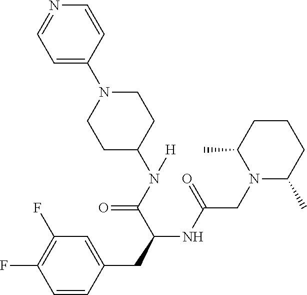 [SMILES: C[C@@H]1CCC[C@H](C)N1CC(=O)N[C@@H](Cc1ccc(F)c(F)c1)C(=O)NC1CCN(c2ccncc2)CC1] | 34.02 |
| C₂₈H₃₇F₂N₅O₂ | |
| 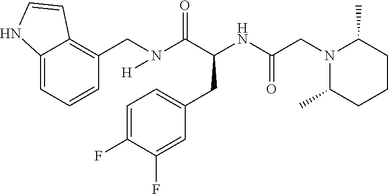 [SMILES: C[C@@H]1CCC[C@H](C)N1CC(=O)N[C@@H](Cc1ccc(F)c(F)c1)C(=O)NCc1cccc2[nH]ccc12] | 34.03 |
| C₂₇H₃₂F₂N₄O₂ | |
| 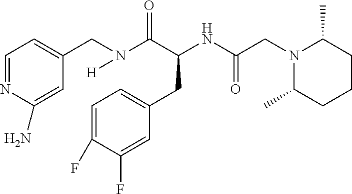 [SMILES: C[C@@H]1CCC[C@H](C)N1CC(=O)N[C@@H](Cc1ccc(F)c(F)c1)C(=O)NCc1ccnc(N)c1] | 34.04 |
| C₂₄H₃₁F₂N₅O₂ | |
| 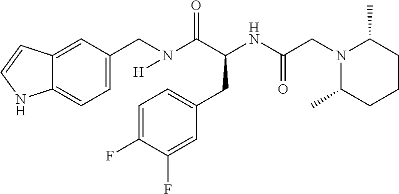 [SMILES: C[C@@H]1CCC[C@H](C)N1CC(=O)N[C@@H](Cc1ccc(F)c(F)c1)C(=O)NCc1ccc2[nH]ccc2c1] | 34.05 |
| C₂₇H₃₂F₂N₄O₂ | |
| 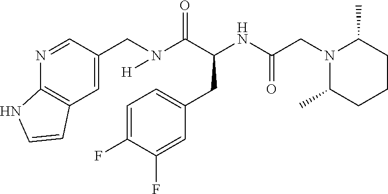 [SMILES: C[C@@H]1CCC[C@H](C)N1CC(=O)N[C@@H](Cc1ccc(F)c(F)c1)C(=O)NCc1cnc2[nH]ccc2c1] | 34.06 |
| C₂₆H₃₁F₂N₅O₂ | |
| 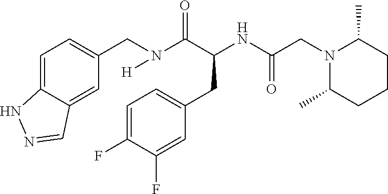 [SMILES: C[C@@H]1CCC[C@H](C)N1CC(=O)N[C@@H](Cc1ccc(F)c(F)c1)C(=O)NCc1ccc2[nH]ncc2c1] | 34.07 |
| C₂₆H₃₁F₂N₅O₂ | |
| 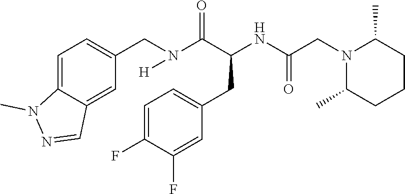 [SMILES: C[C@@H]1CCC[C@H](C)N1CC(=O)N[C@@H](Cc1ccc(F)c(F)c1)C(=O)NCc1ccc2c(cnn2C)c1] | 34.08 |
| C₂₇H₃₃F₂N₅O₂ | |

TABLE 16

| Structure | Example |
|---|---|
| Molecular formula | No. |
| 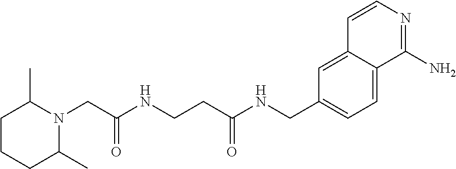 [SMILES: CC1CCCC(C)N1CC(=O)NCCC(=O)NCc1ccc2c(N)nccc2c1] | 39.01 |
| C₂₂H₃₁N₅O₂ | |
| 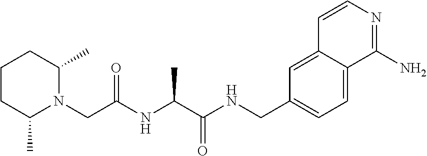 [SMILES: C[C@H](NC(=O)CN1[C@H](C)CCC[C@@H]1C)C(=O)NCc1ccc2c(N)nccc2c1] | 39.02 |
| C₂₂H₃₁N₅O₂ | |
| 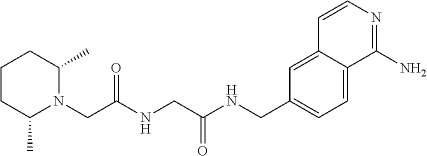 [SMILES: C[C@@H]1CCC[C@H](C)N1CC(=O)NCC(=O)NCc1ccc2c(N)nccc2c1] | 39.03 |
| C₂₁H₂₉N₅O₂ | |
| 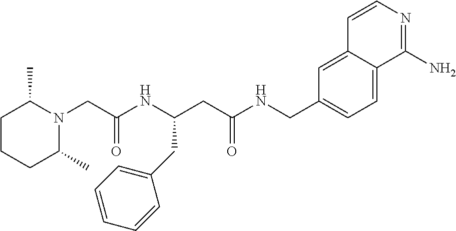 [SMILES: C[C@@H]1CCC[C@H](C)N1CC(=O)N[C@H](CC(=O)NCc1ccc2c(N)nccc2c1)Cc1ccccc1] | 39.04 |
| C₂₉H₃₇N₅O₂ | |
| 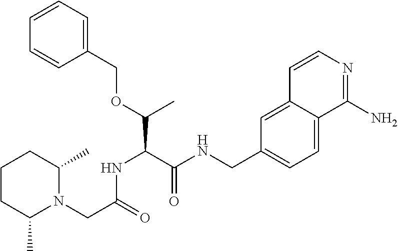 [SMILES: CC(OCc1ccccc1)[C@H](NC(=O)CN1[C@H](C)CCC[C@@H]1C)C(=O)NCc1ccc2c(N)nccc2c1] | 39.05 |
| C₃₀H₃₉N₅O₃ | |
| 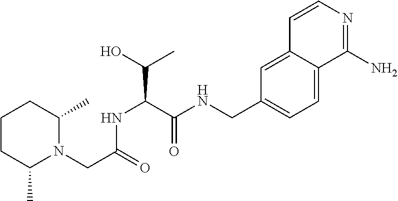 [SMILES: CC(O)[C@H](NC(=O)CN1[C@H](C)CCC[C@@H]1C)C(=O)NCc1ccc2c(N)nccc2c1] | 39.06 |
| C₂₃H₃₃N₅O₃ | |
| 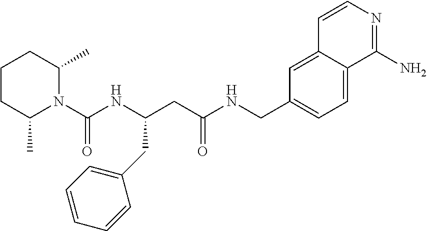 [SMILES: C[C@@H]1CCC[C@H](C)N1C(=O)N[C@H](CC(=O)NCc1ccc2c(N)nccc2c1)Cc1ccccc1] | 39.07 |
| C₂₈H₃₅N₅O₂ | |
| 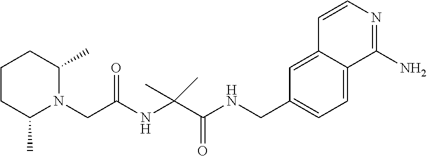 [SMILES: C[C@@H]1CCC[C@H](C)N1CC(=O)NC(C)(C)C(=O)NCc1ccc2c(N)nccc2c1] | 39.08 |
| C₂₃H₃₃N₅O₂ | |
| 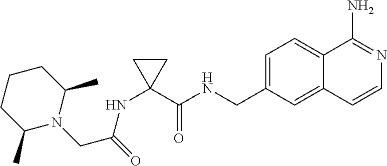 [SMILES: C[C@@H]1CCC[C@H](C)N1CC(=O)NC1(C(=O)NCc2ccc3c(N)nccc3c2)CC1] | 39.09 |
| C₂₃H₃₁N₅O₂ | |
| 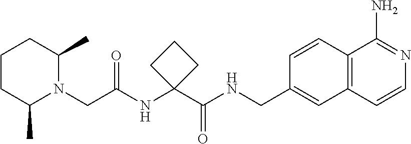 [SMILES: C[C@@H]1CCC[C@H](C)N1CC(=O)NC1(C(=O)NCc2ccc3c(N)nccc3c2)CCC1] | 39.10 |
| C₂₄H₃₃N₅O₂ | |
| 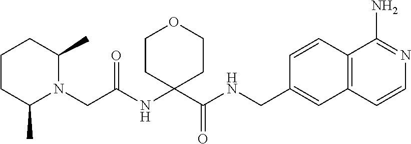 [SMILES: C[C@@H]1CCC[C@H](C)N1CC(=O)NC1(C(=O)NCc2ccc3c(N)nccc3c2)CCOCC1] | 39.11 |
| C₂₅H₃₅N₅O₃ | |

TABLE 17

| Structure | Example |
|---|---|
| Molecular formula | No. |
| 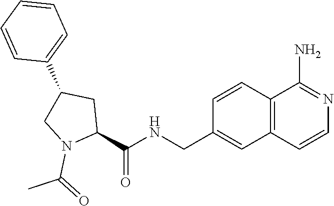 [SMILES: CC(=O)N1C[C@H](c2ccccc2)C[C@H]1C(=O)NCc1ccc2c(N)nccc2c1] | 43.01 |
| C₂₃H₂₄N₄O₂ | |
| 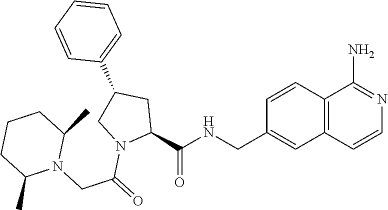 [SMILES: C[C@@H]1CCC[C@H](C)N1CC(=O)N1C[C@H](c2ccccc2)C[C@H]1C(=O)NCc1ccc2c(N)nccc2c1] | 43.02 |
| C₃₀H₃₇N₅O₂ | |
| 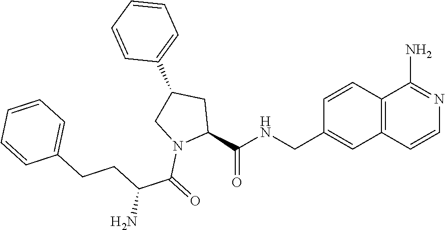 [SMILES: Nc1nccc2cc(CNC(=O)[C@@H]3C[C@@H](c4ccccc4)CN3C(=O)[C@H](N)CCc3ccccc3)ccc12] | 43.03 |
| C₃₁H₃₃N₅O₂ | |
| 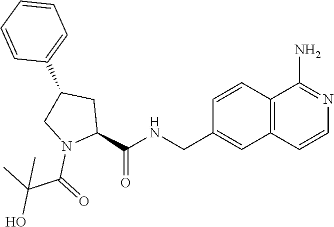 [SMILES: CC(C)(O)C(=O)N1C[C@H](c2ccccc2)C[C@H]1C(=O)NCc1ccc2c(N)nccc2c1] | 43.04 |
| C₂₅H₂₈N₄O₃ | |
| 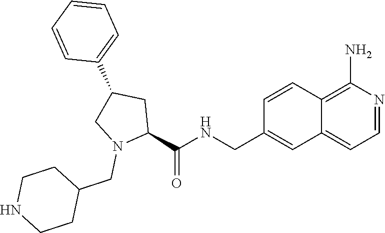 [SMILES: Nc1nccc2cc(CNC(=O)[C@@H]3C[C@@H](c4ccccc4)CN3CC3CCNCC3)ccc12] | 43.05 |
| C₂₇H₃₃N₅O | |
| 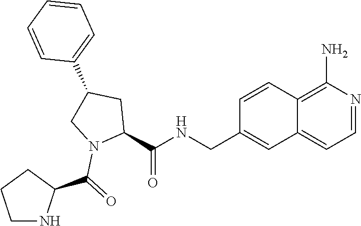 [SMILES: Nc1nccc2cc(CNC(=O)[C@@H]3C[C@@H](c4ccccc4)CN3C(=O)[C@@H]3CCCN3)ccc12] | 43.06 |
| C₂₆H₂₉N₅O₂ | |
| 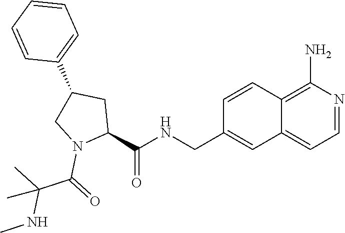 [SMILES: CNC(C)(C)C(=O)N1C[C@H](c2ccccc2)C[C@H]1C(=O)NCc1ccc2c(N)nccc2c1] | 43.07 |
| C₂₆H₃₁N₅O₂ | |

TABLE 18

| Structure | Example |
|---|---|
| Molecular formula | No. |
| 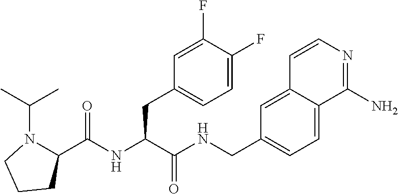 [SMILES: CC(C)N1CCC[C@@H]1C(=O)N[C@@H](Cc1ccc(F)c(F)c1)C(=O)NCc1ccc2c(N)nccc2c1] | 44.01 |
| C₂₇H₃₁F₂N₅O₂ | |
| 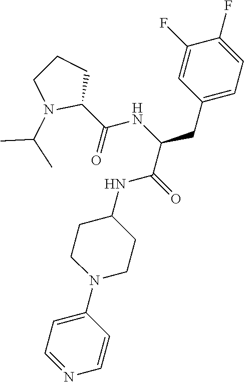 [SMILES: CC(C)N1CCC[C@@H]1C(=O)N[C@@H](Cc1ccc(F)c(F)c1)C(=O)NC1CCN(c2ccncc2)CC1] | 44.02 |
| C₂₇H₃₅F₂N₅O₂ | |
| 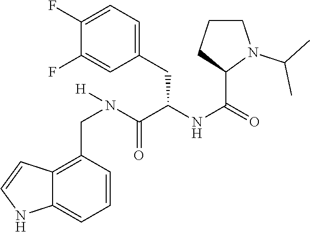 [SMILES: CC(C)N1CCC[C@@H]1C(=O)N[C@@H](Cc1ccc(F)c(F)c1)C(=O)NCc1cccc2[nH]ccc12] | 44.03 |
| C₂₆H₃₀F₂N₄O₂ | |
| 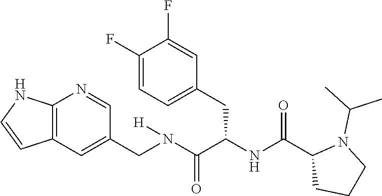 [SMILES: CC(C)N1CCC[C@@H]1C(=O)N[C@@H](Cc1ccc(F)c(F)c1)C(=O)NCc1cnc2[nH]ccc2c1] | 44.04 |
| C₂₅H₂₉F₂N₅O₂ | |
| 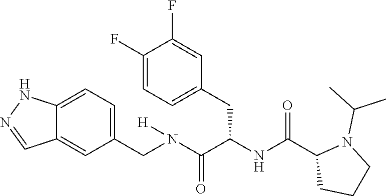 [SMILES: CC(C)N1CCC[C@@H]1C(=O)N[C@@H](Cc1ccc(F)c(F)c1)C(=O)NCc1ccc2[nH]ncc2c1] | 44.05 |
| C₂₅H₂₉F₂N₅O₂ | |
| 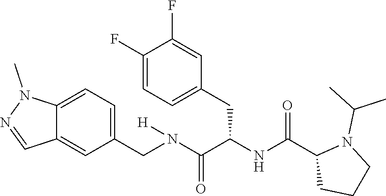 [SMILES: CC(C)N1CCC[C@@H]1C(=O)N[C@@H](Cc1ccc(F)c(F)c1)C(=O)NCc1ccc2c(cnn2C)c1] | 44.06 |
| C₂₆H₃₁F₂N₅O₂ | |

TABLE 19

| Structure | Example |
|---|---|
| Molecular formula | No. |
| 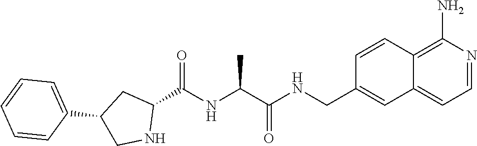 [SMILES: C[C@H](NC(=O)[C@H]1C[C@@H](c2ccccc2)CN1)C(=O)NCc1ccc2c(N)nccc2c1] | 45.01 |
| C₂₄H₂₇N₅O₂ | |
| 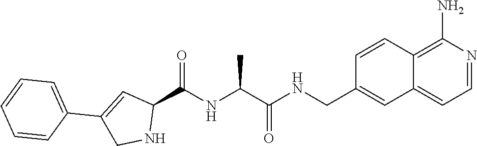 [SMILES: C[C@H](NC(=O)[C@@H]1C=C(c2ccccc2)CN1)C(=O)NCc1ccc2c(N)nccc2c1] | 45.02 |
| C₂₄H₂₅N₅O₂ | |
| 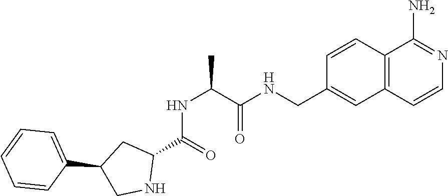 [SMILES: C[C@H](NC(=O)[C@H]1C[C@H](c2ccccc2)CN1)C(=O)NCc1ccc2c(N)nccc2c1] | 45.03 |
| C₂₄H₂₇N₅O₂ | |
| 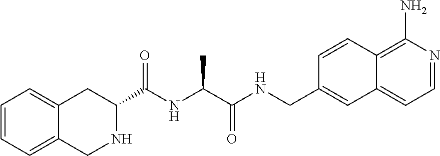 [SMILES: C[C@H](NC(=O)[C@H]1Cc2ccccc2CN1)C(=O)NCc1ccc2c(N)nccc2c1] | 45.04 |
| C₂₃H₂₅N₅O₂ | |
| 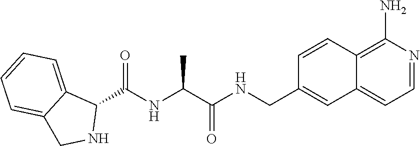 [SMILES: C[C@H](NC(=O)[C@@H]1NCc2ccccc21)C(=O)NCc1ccc2c(N)nccc2c1] | 45.05 |
| C₂₂H₂₃N₅O₂ | |

TABLE 20

| Structure | Example |
|---|---|
| Molecular formula | No. |
| 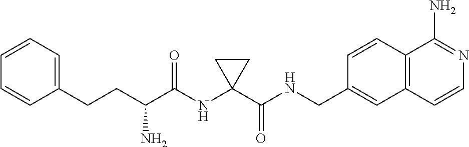 [SMILES: Nc1nccc2cc(CNC(=O)C3(NC(=O)[C@H](N)CCc4ccccc4)CC3)ccc12] | 54.01 |
| C₂₄H₂₇N₅O₂ | |
| 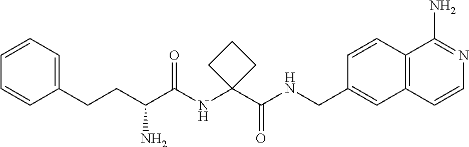 [SMILES: Nc1nccc2cc(CNC(=O)C3(NC(=O)[C@H](N)CCc4ccccc4)CCC3)ccc12] | 54.02 |
| C₂₅H₂₉N₅O₂ | |
| 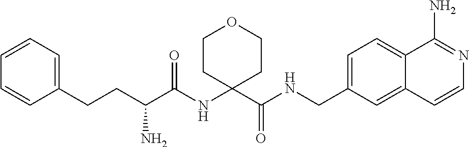 [SMILES: Nc1nccc2cc(CNC(=O)C3(NC(=O)[C@H](N)CCc4ccccc4)CCOCC3)ccc12] | 54.03 |
| C₂₆H₃₁N₅O₃ | |

TABLE 21

| Structure | Example |
|---|---|
| Molecular formula | No. |
| 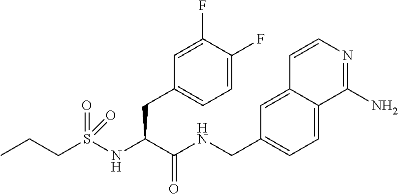 [SMILES: CCCS(=O)(=O)N[C@@H](Cc1ccc(F)c(F)c1)C(=O)NCc1ccc2c(N)nccc2c1] | 55.01 |
| C₂₂H₂₄F₂N₄O₃S | |
| 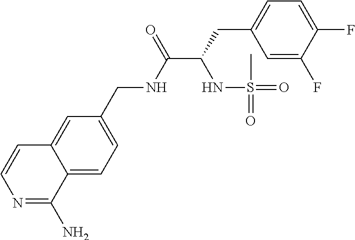 [SMILES: CS(=O)(=O)N[C@@H](Cc1ccc(F)c(F)c1)C(=O)NCc1ccc2c(N)nccc2c1] | 55.02 |
| C₂₀H₂₀F₂N₄O₃S | |
| 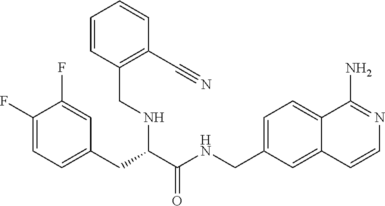 [SMILES: N#Cc1ccccc1CN[C@@H](Cc1ccc(F)c(F)c1)C(=O)NCc1ccc2c(N)nccc2c1] | 55.03 |
| C₂₇H₂₃F₂N₅O | |
| 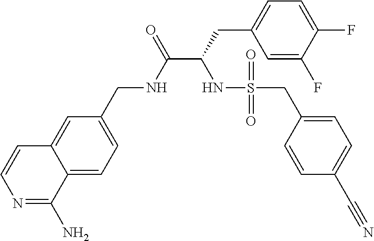 [SMILES: N#Cc1ccc(CS(=O)(=O)N[C@@H](Cc2ccc(F)c(F)c2)C(=O)NCc2ccc3c(N)nccc3c2)cc1] | 55.04 |
| C₂₇H₂₃F₂N₅O₃S | |
| 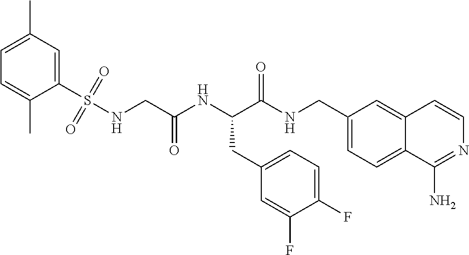 [SMILES: Cc1ccc(C)c(S(=O)(=O)NCC(=O)N[C@@H](Cc2ccc(F)c(F)c2)C(=O)NCc2ccc3c(N)nccc3c2)c1] | 55.05 |
| C₂₉H₂₉F₂N₅O₄S | |

TABLE 22

| Structure | Example |
|---|---|
| Molecular formula | No. |
| 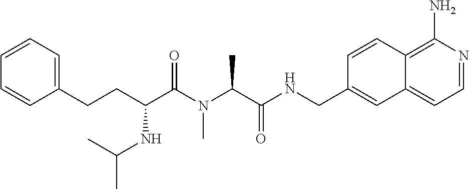 [SMILES: CC(C)N[C@H](CCc1ccccc1)C(=O)N(C)[C@@H](C)C(=O)NCc1ccc2c(N)nccc2c1] | 65.01 |
| C₂₇H₃₅N₅O₂ | |
| 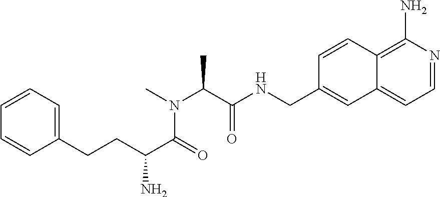 [SMILES: C[C@@H](C(=O)NCc1ccc2c(N)nccc2c1)N(C)C(=O)[C@H](N)CCc1ccccc1] | 65.02 |
| C₂₄H₂₉N₅O₂ | |

TABLE 23

| Structure | Example |
|---|---|
| Molecular formula | No. |
| 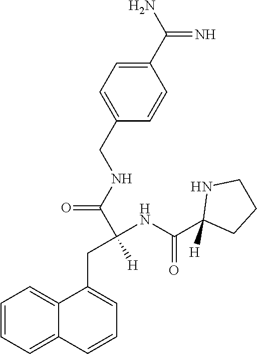 [SMILES: N=C(N)c1ccc(CNC(=O)[C@H](Cc2cccc3ccccc23)NC(=O)[C@H]2CCCN2)cc1] | 71.01 |
| C₂₆H₂₉N₅O₂ | |
| 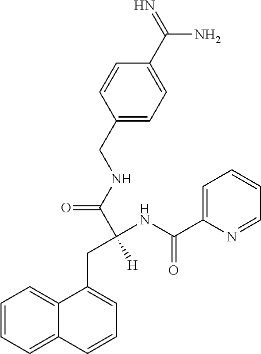 [SMILES: N=C(N)c1ccc(CNC(=O)[C@H](Cc2cccc3ccccc23)NC(=O)c2ccccn2)cc1] | 71.02 |
| C₂₇H₂₅N₅O₂ | |
| 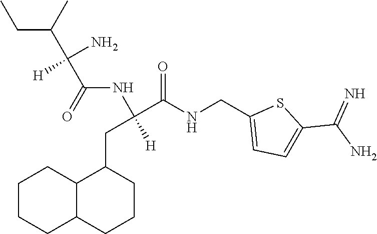 [SMILES: CCC(C)[C@@H](N)C(=O)N[C@H](CC1CCCC2CCCCC21)C(=O)NCc1ccc(C(=N)N)s1] | 71.03 |
| C₂₅H₄₁N₅O₂S | |
| 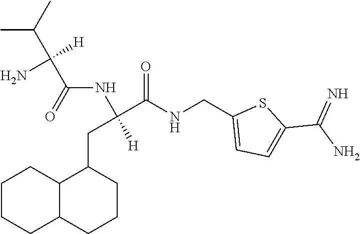 [SMILES: CC(C)[C@H](N)C(=O)N[C@H](CC1CCCC2CCCCC21)C(=O)NCc1ccc(C(=N)N)s1] | 71.04 |
| C₂₄H₃₉N₅O₂S | |
| 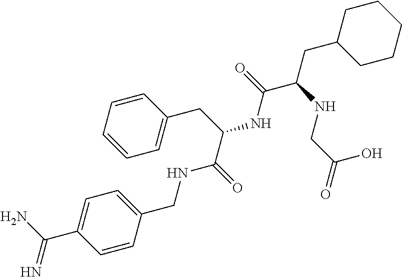 [SMILES: N=C(N)c1ccc(CNC(=O)[C@H](Cc2ccccc2)NC(=O)[C@@H](CC2CCCCC2)NCC(=O)O)cc1] | 71.05 |
| C₂₈H₃₇N₅O₄ | |

### Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 Inh HAE).

The disease or condition in which FXIIa activity is implicated can be selected from vascular hyperpermeability, stroke including ischemic stroke and haemorrhagic accidents; retinal edema; diabetic retinopathy; DME; retinal vein occlusion; and AMD. These conditions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

### Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A and by S. Patel in Retina, 2009 June; 29 (6 Suppl):S45-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®) and lanadelumab (Takhzyro®); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor VIIa inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### Definitions

As noted above, the term “alkoxy” is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R7)₂ and fluoro. Examples of such alkoxy groups include, but are not limited to, C1-methoxy, C₂-ethoxy, C₃-n-propoxy and C₄-n-butoxy for linear alkoxy, and C₃-iso-propoxy, and C₄-sec-butoxy and tert-butoxy for branched alkoxy, optionally substituted as noted above. More specifically, alkoxy can be linear groups of between 1 and 4 carbon atoms (C₁-C₄), more specifically, between 1 and 3 carbon atoms (C₁-C₃). More specifically, alkoxy can be branched groups of between 3 and 4 carbon atoms (C₃-C₄), optionally substituted as noted above.

As noted above, the term “alkyl” is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₁-C₆)alkoxy, OH, —NR8R9, —NHCOCH₃, —CO(heterocyclyl^{b}), —COOR8, —CONR8R9, CN, CF₃, halo, oxo and heterocyclyl^{b}. As noted above, the term “alkyl^{b}” is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₁-C₆)alkoxy, OH, —N(R7)₂, —NHCOCH₃, CF₃, halo, oxo and cyclopropane. Examples of such alkyl or alkyl^{b} groups include, but are not limited, to C₁-methyl, C₂-ethyl, C₃-propyl and C₄-n-butyl, C₃-iso-propyl, C₄-sec-butyl, C₄-isobutyl, C₄-tert-butyl and C₅-neo-pentyl), optionally substituted as noted above. More specifically, “alkyl” or “alkyl^{b}” can be a linear saturated hydrocarbon having up to 4 carbon atoms (C₁-C₄) or a branched saturated hydrocarbon of between 3 and 4 carbon atoms (C₃-C₄), optionally substituted as noted above, which is herein called “small alkyl” or “small alkyl^{b}”, respectively. Preferably, “alkyl” or “alkyl^{b}” can be defined as a “small alkyl” or “small alkyl^{b}”.

As noted above, the term “alkylene” is a bivalent linear saturated hydrocarbon having 1 to 5 carbon atoms (C₁-C₅); alkylene may optionally be substituted with 1 or 2 substituents independently selected from alkyl, (C₁-C₆)alkoxy, OH, CN, CF₃ and halo. More specifically, alkylene can be a bivalent linear saturated hydrocarbon having 2 to 4 carbon atoms (C₂-C₄), more specifically having 2 to 3 carbon atoms (C₂-C₃), optionally substituted as noted above.

As noted above, the term “aryl” is phenyl, biphenyl or naphthyl; aryl may be optionally substituted with 1, 2 or 3 substituents independently selected from alkyl, alkoxy, OH, —SO₂CH₃, halo, —SO₂NR8R9, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR8, —CONR8R9, —(CH₂)O₃—NR8R9, OCF₃ and CF₃; or two adjacent carbon ring atoms on the aryl may be optionally linked by a heteroalkylene to form a non-aromatic ring containing 5, 6, or 7 ring members which may be optionally substituted with OH; or optionally wherein two adjacent ring atoms on aryl are linked to form a 5- or 6-membered aromatic ring containing 1 or 2 heteroatoms that are selected from N, NR10, S, and O. Preferably, “aryl” is phenyl, biphenyl or naphthyl; aryl may be optionally substituted with 1, 2 or 3 substituents independently selected from alkyl, alkoxy, OH, —SO₂CH₃, halo, —(CH₂)₀₋₃-heteroaryl^{b}, —(CH₂)₀₋₃—NR8R9, CN, —SO₂NR8R9; or where two adjacent carbon ring atoms on the aryl are be optionally linked by a heteroalkylene to form a non-aromatic ring containing 5, 6, or 7 ring members which may be optionally substituted with OH.

As noted above, the term “aryl^{b}” is phenyl, biphenyl or naphthyl, which may be optionally substituted with 1, 2 or 3 substituents independently selected from methyl, ethyl, propyl, isopropyl, alkoxy, OH, —SO₂CH₃, N(R7)₂, halo, CN, and CF₃; or two adjacent carbon ring atoms on the aryl may be optionally linked by a heteroalkylene to form a non-aromatic ring containing 5, 6, or 7 ring members.

As noted above, the term “cycloalkyl” is monocyclic saturated hydrocarbon ring of between 3 and 6 carbon atoms (C₃-C₆); cycloalkyl may optionally be substituted with 1 or 2 substituents independently selected from methyl, ethyl, propyl, isopropyl, methoxy, ethoxy, propoxy, isopropoxy, OH, CN, CF₃ and halo; optionally wherein two adjacent ring atoms on cycloalkyl are linked to form a 5- or 6-membered saturated hydrocarbon ring. Examples of suitable monocyclic cycloalkyl groups include cyclopropyl, cyclobutyl, cyclopentyl, and cyclohexyl), optionally substituted as noted above.

As noted above, “halo” can be selected from F, Cl, Br and I. More specifically, halo can be selected from Cl and F.

As noted above, the term “heteroalkylene” is a bivalent linear saturated hydrocarbon having 2 to 5 carbon atoms (C₂-C₅), wherein 1 or 2 of the 2 to 5 carbon atoms are replaced with NR10, S, or O; heteroalkylene may optionally be substituted with 1 or 2 substituents independently selected from alkyl, (C₁-C₆)alkoxy, OH, CN, CF₃ and halo. More specifically, heteroalkylene can be a bivalent linear saturated hydrocarbon having 2 to 4 carbon atoms (C₂-C₄), wherein at least one of the 2 to 4 carbon atoms is replaced with NR10, S, or O; or having 2 to 3 carbon atoms (C₂-C₃), wherein at least one of the 2 to 3 carbon atoms is replaced with NR10, S, or O, each optionally substituted as noted above.

As noted above, the term “heteroaryl” is a 5- or 6-membered carbon-containing aromatic ring containing one, two or three ring members that are selected from N, NR10, S, and O; heteroaryl may be optionally substituted with 1, 2 or 3 substituents independently selected from alkyl, alkoxy, heteroaryl^{b}, phenyl, cycloalkyl, OH, OCF₃, halo, heterocyclyl^{b}, CN, and CF₃. As noted above, the term “heteroaryl^{b}” is a 5- or 6-membered carbon-containing aromatic ring containing one, two or three ring members that are selected from N, NR10, S, and O; heteroaryl^{b} may be optionally substituted with 1, 2 or 3 substituents independently selected from methyl, ethyl, propyl, isopropyl, alkoxy, OH, OCF₃, COOCH₃, COOCH₂CH₃, COO—(CH₂)₂—CH₃, COO-(iPr), halo, CN, and CF₃. Examples of suitable heteroaryl or heteraryl^{b} are thiophene, furan, pyrrole, pyrazole, imidazole, oxazole, isoxazole, thiazole, isothiazole, triazole, oxadiazole, thiadiazole, pyridine, pyridazine, pyrimidine, and pyrazine, optionally substituted as noted above.

As noted above, the term “heterocyclyl” is a 4-, 5-, 6-, or 7-membered carbon-containing non-aromatic ring containing one, two, three, or four ring members that are selected from N, NR10, S, SO, SO₂ and O; heterocyclyl may be optionally substituted with 1, 2, 3, or 4 substituents independently selected from alkyl, alkoxy, aryl^{b}, OH, OCF₃, halo, oxo, CN, NR8R9, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two ring atoms on heterocyclyl are linked with an heteroalkylene to form a non-aromatic ring containing 5, 6, or 7 ring members; or optionally wherein two adjacent ring atoms on heterocyclyl are linked to form a 5- or 6-membered aromatic ring which may optionally contain 1 or 2 heteroatoms that are selected from N, NR10, S, and O. More specifically, “heterocyclyl” can be a 5-, 6-, or 7-membered carbon-containing non-aromatic ring, wherein one or two of the ring members are independently selected from N, NR10 and O, which may be optionally substituted as above, or wherein two ring atoms may be linked as above.

As noted above, the term “heterocyclyl^{b}” is a 4-, 5-, 6-, or 7-membered carbon-containing non-aromatic ring containing one, two or three ring members that are selected from N, NR7, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃. More specifically, “heterocyclyl^{b}” can be a non-aromatic ring containing one, two or three ring members that are selected from N, NR7, and O, which can be optionally substituted as above.

As noted above, the term “O-linked”, such as in “O-linked hydrocarbon residue”, means that the hydrocarbon residue is joined to the remainder of the molecule via an oxygen atom.

In groups such as —(CH₂)₁₋₃-aryl, “-” denotes the point of attachment of the substituent group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

In the context of the present invention, references herein to “treatment” include references to curative, palliative and prophylactic treatment.

### General Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

The compounds of the invention can be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration. If administered by intra-vitreal injection a lower dose of between 0.0001 mg (0.1 μg) and 0.2 mg (200 μg) per eye is envisaged, or between 0.0005 mg (0.5 μg) and 0.05 mg (50 μg) per eye.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively, the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively, a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively, where a hydroxy group is protected as a benzyl ether, deprotection conditions comprise hydrogenation with a palladium catalyst under a hydrogen atmosphere.

The compounds according to general formula I can be prepared using conventional synthetic methods for example, but not limited to, the routes outlined in Schemes 1-3.

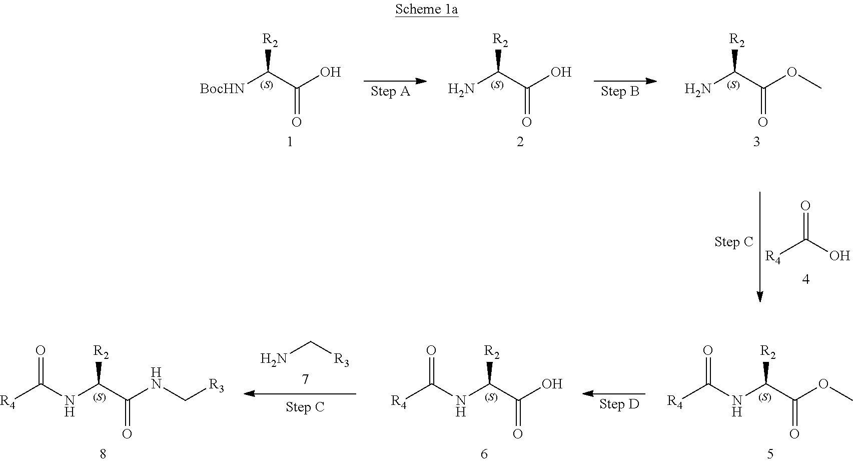

In Scheme 1a, the Boc protecting group is removed (Step A) using acidic conditions such as trifluoroacetic acid or HCl to give compound 2. Typically, this intermediate would be isolated in the form of the acid salt, for example the trifluoroacetate or HCl. The acid 2 is reacted with methanol (Step B) typically via the acid chloride using thionyl chloride to give ester 3. Alternatively, the methyl ester formation can take place using (Diazomethyl)trimethylsilane. The amine (or salt) 3 is coupled to acid 4 (Step C) to give compound 5. This coupling is typically carried out using standard coupling conditions such as hydroxybenzotriazole (HOBt) and carbodiimide such as water soluble carbodiimide in the presence of an organic base. Other standard coupling methods include the reaction of acids with amines in the presence of 2-(1H-benzotriazole-1-yl)-1,1,3,3-tetramethylaminium hexafluorophosphate (HBTU) or benzotriazole-1-yl-oxy-tris-pyrrolidino-phosphonium hexafluorophosphate (PyBOP) or bromo-trispyrrolidino-phosphonium hexafluorophosphate (PyBroP) or 2-(3H-[1,2,3]triazolo[4,5-b]pyridin-3-yl)-1,1,3,3-tetramethylisouronium hexafluorophosphate(V) (HATU), or 1-ethyl-3-(3-dimethylaminopropyl)carbodiimide (EDC) in the presence of organic bases such as triethylamine, diisopropylethylamine or N-methylmorpholine. Alternatively, the amide formation can take place via an acid chloride in the presence of an organic base. Such acid chlorides can be formed by methods well known in the literature, for example reaction of the acid with oxalyl chloride or thionyl chloride. Alternatively, the carboxylic acid can be activated using 1,1′-carbonyldiimidazole (CDI) and then amine added. The ester is hydrolysed (Step D) using standard literature conditions such as NaOH, KOH, LiOH, or TMSOK. The acid (or salt) 6 is coupled to amine (or salt) 7 to give compound 8 repeating step C conditions. The amine 7 may be commercially available or prepared from readily available starting materials using methods known in the art, or as detailed in specific examples herein. Depending on R₃ the final compound may require removal of protecting groups using methods known in the art.

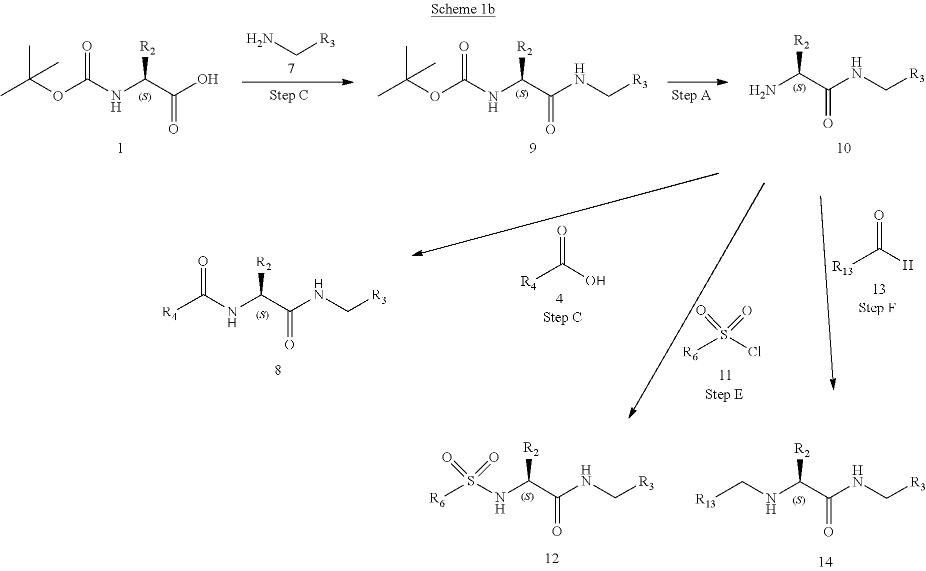

In Scheme 1b, the order in which the steps are completed is reversed but they utilise the same synthetic methodology as described for Scheme 1a. In addition, the final step via this route is optionally diverse. It can be an amide formation as described previously (Step C) to give compound 8. It can also be a sulfonamide formation by reaction of compound 10 with a sulfonyl chloride 11 in the presence of a base such as triethylamine or N,N-diisopropylethylamine (DIPEA) (Step E) to give sulfonamide 12. 4-Dimethylaminopyridine (DMAP) may also be added. Alternatively, alkylation of the amine (Step F) may be carried out using standard conditions for such a transformation. For example, amine 10 is treated with formaldehyde (37% in water) followed by the addition of a reducing agent such as sodium triacetoxyborohydride to give compound 14. Alternative alkylations may be carried out by use of the appropriate alkanone, for example amine 4 is treated with the alkanone, for example acetone, in an organic solvent such as DCM followed by the addition of a reducing agent such as sodium triacetoxyborohydride to give compound 14. Alternative reducing agents include sodium borohydride and sodium cyanoborohydride. Depending on R₃, the final compound may require removal of protecting groups using methods known in the art.

When the acid 4 in Scheme 1 above is an amino acid it may be prepared from readily available starting materials using methods known in the art, for example as shown in Scheme 2.

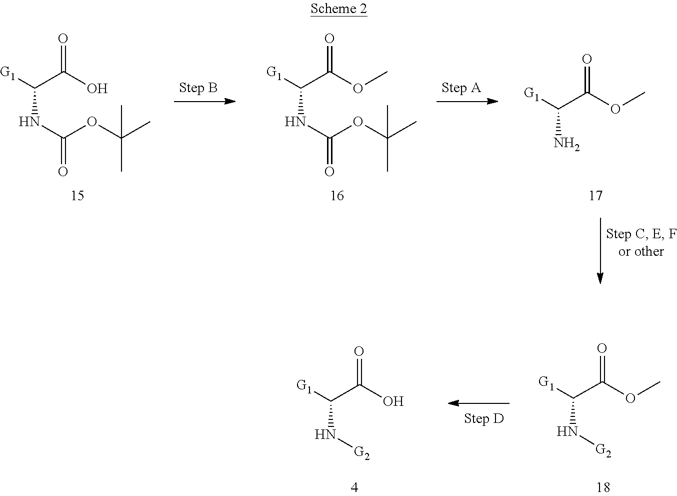

The acid 15 is protected via the methyl ester using standard conditions as for Step B to give compound 16. The Boc protecting group is removed as for Step A to give amine 17 which may be isolated as the salt or free amine. The amine 17 may be reacted under a variety of literature conditions to form compound 18 including, but not limited to sulfonylation, reductive amination, alkylation, Buchwald coupling, Chan-Lam coupling and amide coupling.

Alternatively, substitution of the amine can take place later in the synthetic sequence as shown in Scheme 3a, 3b and 3c.

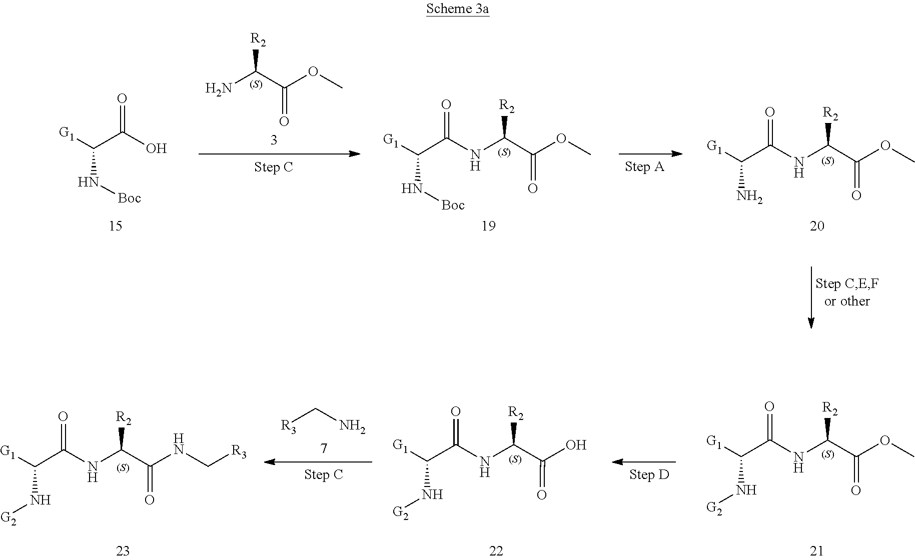

The protected amino acid 15 is reacted with amino acid methyl ester 3 under typical amide coupling conditions (Step C) to give compound 19. The Boc protecting group is removed (Step A) using acidic conditions such as trifluoroacetic acid or hydrogen chloride to give amine 20. Typically this intermediate may be isolated in the form of the acid salt, for example the trifluoroacetate or the hydrochloride. The amine 20 may be reacted under a variety of literature conditions to form compound 21 including, but not limited to, sulfonylation, reductive amination, alkylation, Buchwald coupling, Chan-Lam coupling and amide coupling. The ester is hydrolysed (Step D) using standard literature conditions such as LiOH or TMSOK. The acid (or salt) 22 is coupled to amine 7 (or salt) (Step C) to give compound 23.

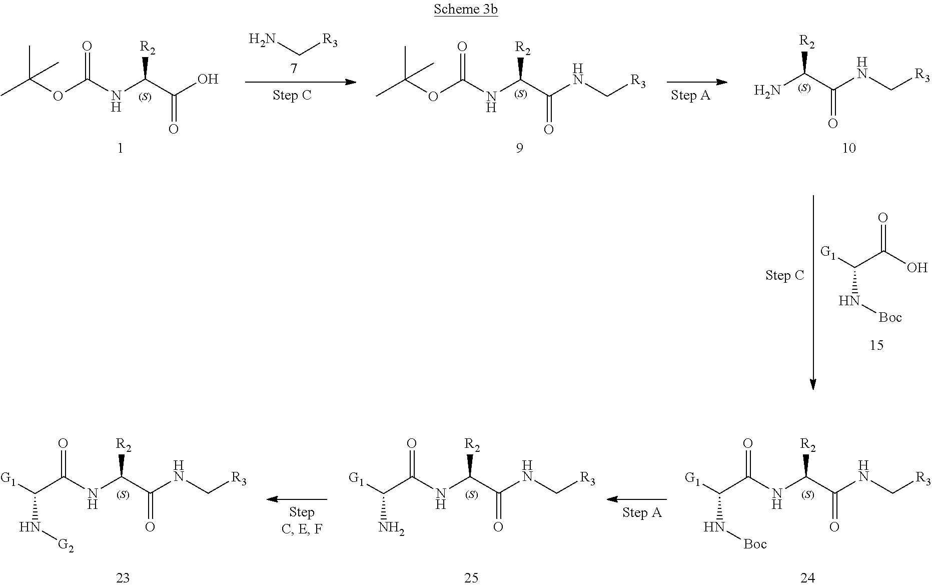

The amino acid 1 is coupled to amine 7 (or salt) (Step C) to give compound 9. The Boc protecting group is removed (Step A) using acidic conditions such as trifluoroacetic acid or hydrogen chloride to give amine 10. Typically this intermediate would be isolated in the form of the acid salt, for example the trifluoroacetate or the hydrochloride. The amino acid 10 is coupled to amino acid 15 (Step C) to give compound 24. The Boc protecting group is removed following the previous procedure (Step A) to give the amine 25. Alkylation of the amine 25 may be carried out using standard conditions for such a transformation, for example reductive alkylation (Step F). They may be carried out by use of the appropriate alkanone, for example amine 25 is treated with the alkanone, for example acetone, in an organic solvent such as DCM followed by the addition of a reducing agent such as sodium triacetoxyborohydride to give compound 23. Alternative reducing agents include sodium borohydride and sodium cyanoborohydride. The amine 25 may also undergo amide coupling (Step C), and sulfonylation (Step E), as previously described.

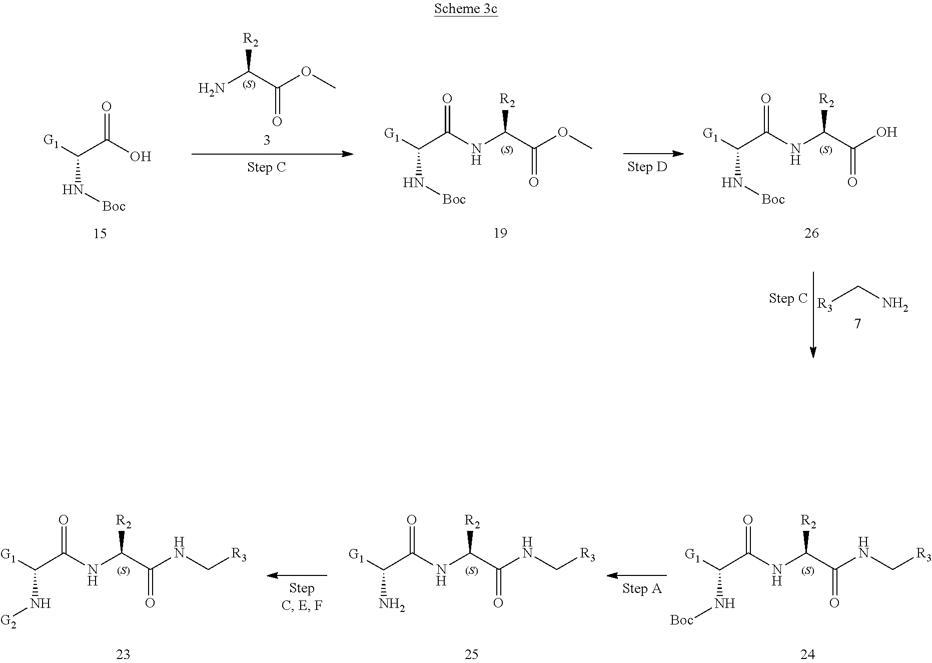

The protected amino acid 15 is reacted with amino acid methyl ester 3 under typical amide coupling conditions (Step C) to give compound 19. The ester is hydrolysed (Step D) using standard literature conditions such as LiOH or TMSOK. The acid (or salt) 26 is coupled to amine 7 (or salt) (Step C) to give compound 24. The Boc protecting group is removed (Step A) using acidic conditions such as trifluoroacetic acid or hydrogen chloride to give amine 25. Typically this intermediate would be isolated in the form of the acid salt, for example the trifluoroacetate or the hydrochloride. The amine 25 may be reacted under a variety of literature conditions to form compound 23 including, but not limited to, amide coupling (Step C), sulfonylation (Step E) and reductive amination (Step F), as previously described.

In the synthetic routes described in Schemes 1-3 above, the protecting group strategy is exemplified by the use of a Boc group. It will be recognised that alternative protecting groups may be utilised in these synthetic routes as have already been discussed above.

### EXAMPLES

The invention is illustrated by the following non-limiting examples in which the following abbreviations and definitions are used:

| | |
|---|---|
| Aq | Aqueous solution |
| AIBN | Azobisisobutyronitrile |
| Boc | tert-Butoxy carbonyl |
| tBu | Tert-Butyl |
| CDI | 1,1′-Carbonyldiimidazole |
| DCM | Dichloromethane |
| DIPEA | N,N-Diisopropylethylamine |
| DMF | N,N-Dimethylformamide |
| DMSO | Dimethyl sulfoxide |
| Eq | Equivalent |
| Et₂O | Diethyl ether |
| Et | Ethyl |
| EtOH | Ethanol |
| EtOAc | Ethyl Acetate |
| HATU | 2-(3H-[1,2,3]triazolo[4,5-b]pyridin-3-yl)-1,1,3,3- |
| | tetramethylisouronium hexafluorophosphate(V) |
| hrs | Hours |
| HOBt | Hydroxybenzotriazole |
| LCMS | Liquid chromatography mass spectrometry |
| Me | Methyl |
| MeCN | Acetonitrile |
| MsCl | Methanesulfonyl chloride |
| MeOH | Methanol |
| Min | Minutes |
| MS | Mass spectrum |
| Ms | Methanesulfonyl |
| NMR | Nuclear magnetic resonance spectrum |
| NMP | N-Methyl-2-pyrrolidone |
| Pet. | Petroleum ether fraction boiling at 60-80° C. |
| Ether | |
| Ph | Phenyl |
| iPr | Iso-propyl |
| nPr | n-Propyl |
| SWFI | Sterile water for injection |
| Rt | room temperature |
| TBDMS | tert-Butyldimethylsilyl |
| TBME | tert-Butyl methyl ether |
| THF | Tetrahydrofuran |
| TEA | Triethylamine |
| TFA | Trifluoroacetic acid |

All reactions were carried out under an atmosphere of nitrogen unless specified otherwise.

¹H NMR spectra were recorded on a Bruker (500 MHz or 400 MHz) spectrometer and reported as chemical shift (ppm).

Molecular ions were obtained using LCMS with appropriate conditions selected from
Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQ mass spectrometer with electospray ionisation in conjunction with a Thermofinnigan Surveyor LC system;
LCMS (Waters Acquity UPLC, C18, Waters X-Bridge UPLC C18, 1.7 μm, 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 μM column, 50×2.1 mm, with a linear gradient 10% to 90% 0.1% HCO₂H/MeCN into 0.1% HCO2H/H2O over 3 minutes, flow rate 1 mL/min. Data was collected using a Waters Acquity UPLC mass spectrometer with quadropole dalton, photodiode array and electrospray ionisation detectors.

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as ChemDraw (PerkinElmer) or the Autonom software provided as part of the ISIS Draw package from MDL Information Systems or the Chemaxon software provided as a component of MarvinSketch or as a component of the IDBS E-WorkBook.

### Examples of the Invention

### General Synthetic Methods

### General Method A: HCl Boc Deprotection

### (S)-2-amino-3-(3,4-difluorophenyl)propanoic Acid

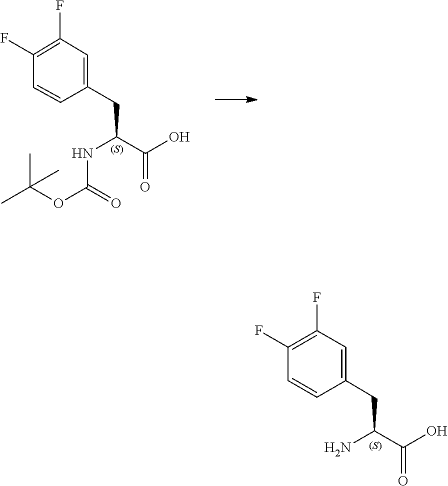

HCl (4M in dioxane) (249 mL, 996 mmol) was added to a solution of (S)-2-((tert-butoxycarbonyl)amino)-3-(3,4-difluorophenyl)propanoic acid (20 g, 66.4 mmol) in dioxane (100 mL) under N₂ and the resulting slurry stirred at rt for 2 hrs. The solvent was removed in vacuo to afford the title compound as an HCl salt as a white solid (16.22 g, 94% yield).

[M+H]⁺=202.1

### General Method B: (i) Methylester Formation (Via Acid Chloride)

### Methyl (S)-2-amino-3-(3,4-difluorophenyl)propanoate

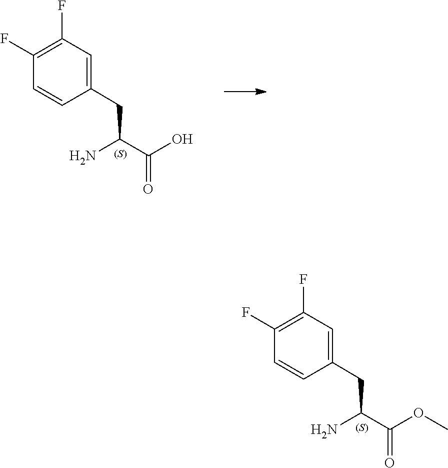

Thionyl chloride (44.2 mL, 606 mmol) was added dropwise over 30 min to a solution of (S)-2-amino-3-(3,4-difluorophenyl)propanoic acid hydrochloride (20.89 g, 80 mmol) in MeOH (250 mL, 6179 mmol) at 0° C. The resulting solution was stirred at 0° C. for 2 hrs then warmed to rt and stirred for 18 hrs before the solvent was removed in vacuo. The resulting beige solid was dissolved in MeOH (60 mL) and a white solid precipitated with diethyl ether (200 mL) and cooling to 0° C. The white solid was collected by filtration to afford the title compound as an HCl salt (19.15 g, 93% yield).

(no mass ion reported)

### General Method B: (ii) Methyl Ester Formation (Via Trimethylsilyldiazomethane)

### Methyl (R)-2-((tert-butoxycarbonyl)amino)-4-phenylbutanoate

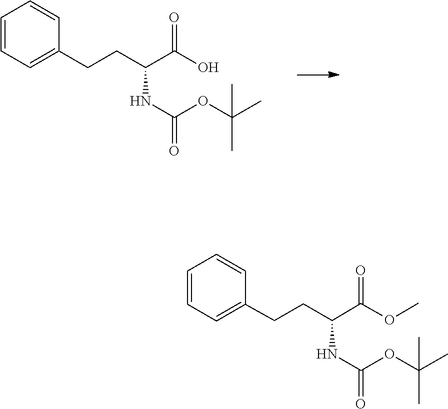 [SMILES: CC(C)(C)OC(=O)N[C@H](CCc1ccccc1)C(=O)O ; COC(=O)[C@@H](CCc1ccccc1)NC(=O)OC(C)(C)C]

A solution of trimethylsilyldiazomethane in hexane (1.1 mL, 2.15 mmol) was added dropwise to a solution of (R)-2-Boc-amino-4-phenyl-butyric acid (300 mg, 1.07 mmol) in anhydrous methanol (1 mL) and DCM (4 mL) at 0° C. The reaction was stirred for 1 h at 0° C. and for a further 2 hrs at rt. The solvent was removed under reduced pressure. The residue was dissolved in a 0.2M HCl solution (10 mL) and washed with ether (3×30 mL). A saturated solution of Na₂CO₃ was added to the aqueous phase until the pH of the solution was basic. The solution was extracted with CHCl₃ (3×20 mL), combined organics were washed with brine (20 mL), dried over Na₂SO₄, filtered and concentrated. The desired product was isolated as a colourless oil and used without further purification.

[M+H+]=294.4

### General Method C: (i) Amide Coupling (HATU)

### (S)-methyl 3-(3,4-difluorophenyl)-2-((R)-1-isopropylpyrrolidine-2-carboxamido)propanoate

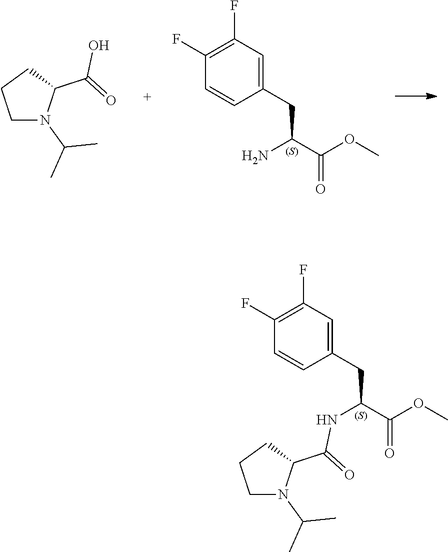

To a suspension of (S)-methyl 2-amino-3-(3,4-difluorophenyl)propanoate hydrochloride (500 mg, 1.99 mmol) in dry DCM (10 mL) under N₂ was added (R)-1-isopropylpyrrolidine-2-carboxylic acid (344 m g, 2.19 mmol) and the reaction mixture cooled to 0° C. DIPEA (1.04 mL, 5.96 mmol) was added, followed by HATU (831 m g, 2.19 mmol). The resulting solution was stirred at 0° C. for 2 hrs. The reaction was concentrated in vacuo and the resulting oil dissolved in EtOAc (150 mL). The organic layer was washed with 1 M HCl (50 mL). The aqueous layer was extracted with EtOAc (2×100 mL). The combined organic extractions were washed sequentially with sat. aq. NaHCO₃ (50 mL), water (50 mL) and brine (50 mL) and dried (Na₂SO₄), filtered and concentrated. The crude product was purified by flash chromatography (0-3% MeOH in DCM) to afford the title compound (540 mg, 72% yield) as a thick colourless oil.

[M+H]⁺=355.3

### General Method C: (ii) Amide Coupling (HOBT)

### (S)-methyl 3-(3,4-difluorophenyl)-2-(2-((2R,6S)-2,6-dimethylpiperidin-1-yl)acetamido)propanoate

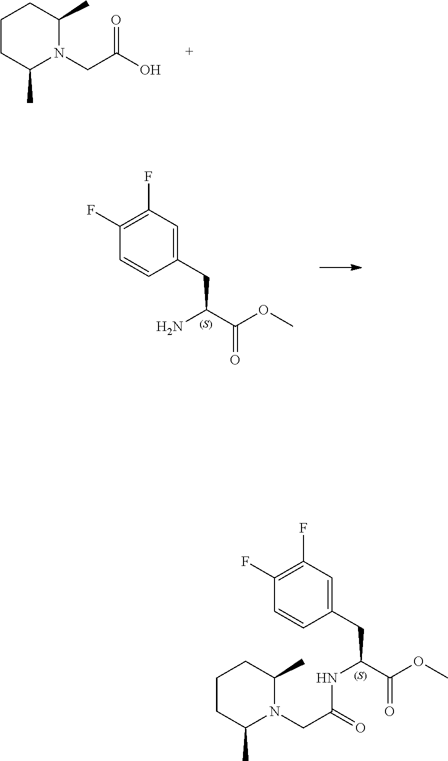

2-((2S,6R)-2,6-dimethylpiperidin-1-yl)acetic acid (0.748 g, 4.37 mmol) was dissolved in dry DCM (50 mL) and dry DMF (2.5 mL) under N₂ then cooled to 0° C. (S)-methyl 2-amino-3-(3,4-difluorophenyl)propanoate hydrochloride (1.00 g, 3.97 mmol) was added to the reaction, followed by HOBT (0.669 g, 4.37 mmol), triethylamine (1.66 mL, 11.92 mmol) and EDC (0.838 g, 4.37 mmol). The resulting solution was allowed to warm to rt and stirred at rt for 18 hrs. The reaction mixture was diluted with CHCl₃ (150 mL) and washed sequentially with sat. aq. NaHCO₃ (50 mL), water (50 mL) and brine (50 mL). The organic layer was dried (Na₂SO₄) and concentrated in vacuo. The crude product was purified by flash chromatography (0-10% MeOH in DCM) to afford the title compound (916 mg, 59% yield) as a thick colourless oil.

[M+H]⁺=369.3

1H-NMR (d6-DMSO) δ: 0.78 (3H, d, J=6.3 Hz), 0.88 (3H, d, J=6.3 Hz), 1.04-1.17 (2H, m), 1.20-1.33 (1H, m), 1.40-1.51 (2H, m), 1.58 (1H, dt, J=12.3, 3.3 Hz), 2.38 (2H, s), 2.83-2.95 (2H, m), 2.95-3.10 (1H, m), 3.10-3.20 (1H, m), 3.65 (3H, s), 4.63 (1H, ddd, J=9.4, 8.3, 5.1 Hz), 6.99-7.13 (1H, m), 7.25-7.39 (2H, m), 7.95 (1H, d, J=8.2 Hz) ppm.

The product was analysed by Chiral HPLC (Lab 1 Bay 4, Diacel Chiralpak IC, 5 um, 4.6×250 mm, 100 min method, 1.0 ml/min, 2-50% EtOH in isohexane (0.2% DEA): 07SDB2, RT=17.7 min, 99% ee @ 254 nm.

### General Method C: (iii) Amide Coupling (HBTU)

### Example 3.42

### (2S)—N-[(1-aminoisoquinolin-6-yl)methyl]-3-(3,4-difluorophenyl)-2-{2-[(2R,6S)-2,6-dimethylpiperidin-1-yl]acetamido}propanamide

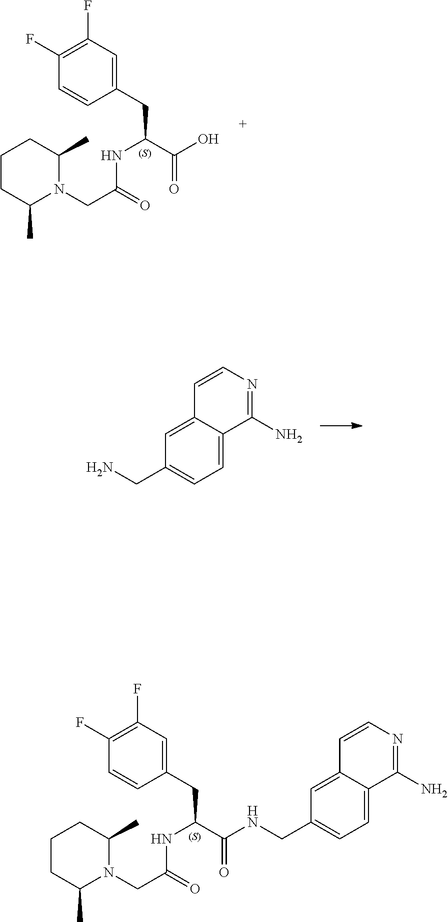

(2S)-3-(3,4-difluorophenyl)-2-{2-[(2R,6S)-2,6-dimethylpiperidin-1-yl]acetamido}propanoic acid 100 mg, 0.28 mmol) was dissolved in DCM (30 mL), HBTU (128 mg, 0.34 mmol) and N,N-diisopropylethyamine (55 mg, 0.42 mmol) was added at rt. After 20 min, 6-aminomethyl-isoquinolin-1-ylamine (54 mg, 0.31 mmol) was added and the reaction mixture stirred at rt for 18 hrs. The reaction mixture was diluted with DCM (50 mL) and washed sequentially with sat. aq. NaHCO₃ (50 mL), water (50 mL) and brine (50 mL). The organic layer was dried (Na₂SO₄) and concentrated in vacuo. The crude product was purified by flash chromatography (0-12% MeOH in CHCl₃) to afford the title compound which was freeze dried from MeCN/water to give a white solid (36 mg, 25% yield).

[M+H]⁺=510.0

1H-NMR (d6-DMSO) δ: 0.78 (3H, d, J=5.8 Hz), 0.88 (3H, t, J=5.4 Hz), 1.12-1.15 (2H, m), 1.23-1.26 (2H, m), 1.45-1; 48 (2H, m), 1.57-1.60 (2H, m), 2.33-2.34 (1H, m), 2.8-2.95 (2H, m), 3.03-3.07 (1H, m), 4.42 (2H, d, J=5.7 Hz), 4.65-4.71 (1H, m), 6.79 (2H, s), 6.81 (1H, d, J=5.8 Hz), 7.05 (1H, s, br), 7.23-7.36 (3H, m), 7.47 (1H, s), 7.76 (1H, d, J=5.9 Hz), 8.12 (1H, s, br), 8.13 (1H, d, J=8.6 Hz), 8.71 (1H, d, J=5.4 Hz).

### General Method D: (i) Ester Hydrolysis (LiOH)

### Lithium (S)-3-(3,4-difluorophenyl)-2-((R)-1-isopropylpyrrolidine-2-carboxamido)propanoate

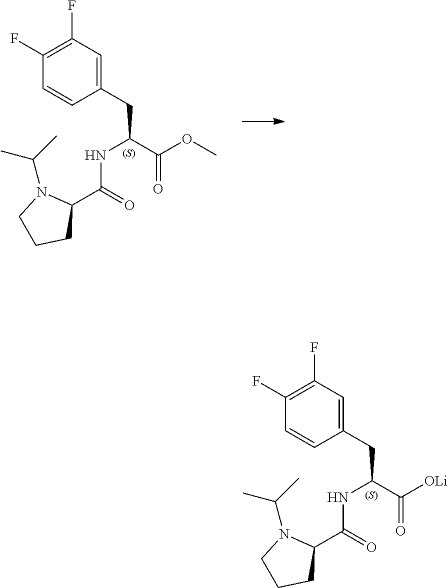

To a solution of lithium hydroxide (1M in water, 277 mg, 11.55 mmol) was added a solution of methyl (S)-3-(3,4-difluorophenyl)-2-((R)-1-isopropylpyrrolidine-2-carboxamido)propanoate (3.91 g, 11 mmol) in THF (37 mL) and water (12 mL) over 10 min at rt. The reaction was stirred for 3 hrs before the solvent was removed in vacuo and the residue azeotroped with MeCN (3×20 mL). The resulting white solid dried in a dessicator overnight to afford the title compound (4 g, 99% yield) as a white solid.

### General Method D: (ii) Ester Hydrolysis (TMSOK)

### Potassium (R)-4-phenyl-2-(pyrrolidin-1-yl)butanoate

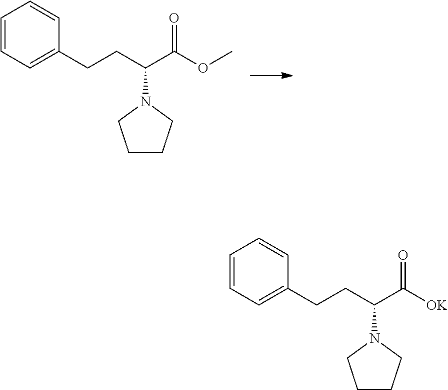

Methyl (R)-4-phenyl-2-(pyrrolidin-1-yl)butanoate (110 mg, 0.45 mmol) was dissolved in dry THF (10 mL) and treated with potassium trimethylsilanolate in THF (724 μL, 1.33 mmol). The reaction mixture was stirred at rt for 18 hrs. The solution was concentrated freeze dried in MeCN and water to afford the title compound as an off white solid (231 mg, 96% yield).

[M+H]⁺=234.33 @ 2.87 mins

### General Method E: Sulfonamide Formation

### Methyl ((R)-2-(methylsulfonamido)-4-phenylbutanoyl)-L-alaninate

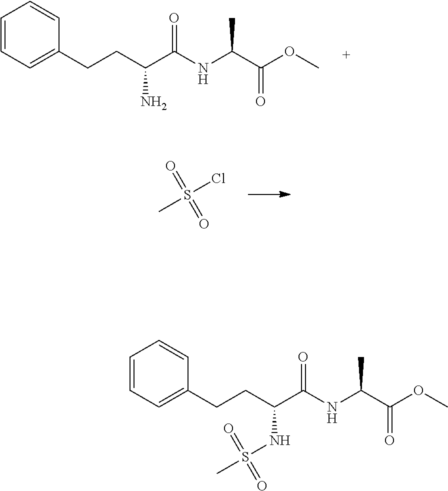

A solution of methyl ((R)-2-amino-4-phenylbutanoyl)-L-alaninate (131 mg, 0.44 mmol) in anhydrous DCM (5 mL) was cooled to 0° C. To this was added and triethylamine (0.18 mL, 1.31 mmol) followed by dropwise addition of methane sulfonylchloride (40 μL, 0.52 mmol). On completion of the addition the ice bath was kept in place and the reaction allowed to warm to rt for 2 hrs. The reaction mixture was diluted with DCM (20 mL) and washed with NaHCO₃ (20 mL), then brine (20 mL), dried over magnesium sulfate, filtered and concentrated. The crude was purified by flash chromatography eluting with (60% EtOAc in Pet. Ether) to afford the desired product (103 mg, 69% yield) as a white solid.

[M+H]⁺=343.0

### General Method F: Reductive Amination

### Methyl ((R)-2-(isopropylamino)-4-phenylbutanoyl)-L-alaninate

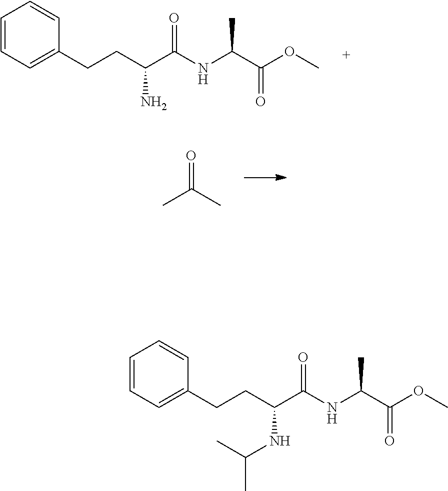

Methyl ((R)-2-amino-4-phenylbutanoyl)-L-alaninate (1.543 g, 5.13 mmol) was dissolved in dry DCM (50 mL) and dry Methanol (10 mL), acetone (415 μL, 5.64 mmol) was added followed by acetic acid (588 μL, 10.26 mmol) and stirred for 60 min. Sodium triacetoxyborohydride (4.348 g, 20.52 mmol) was added portionwise over 10 mins the suspension was stirred for 24 hrs. The reaction mixture was carefully quenched with water and diluted with DCM. The acidic aqueous was separated and washed with DCM (2×20 mL). To the aqueous was then added Na₂CO₃ until the solution reached a basic pH and was then washed with 10% IPA in CHCl₃ (6×25 mL). The combine organics were dried over sodium sulfate and concentrated in vacuo. The crude material was purified by flash chromatography (10% MeOH in DCM) to afford the desired product as a colourless oil (967 mg, 62% yield).

[M+H]⁺=307.1

### Synthesis of Intermediates

### Methyl 6-isopropylpyrimidine-4-carboxylate

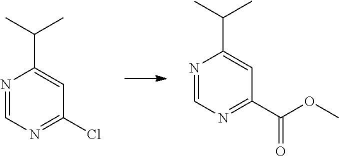

To a microwave vial was added 4-chloro-6-isopropyl-pyrimidine (140 mg, 0.67 mmol), N,N-diisopropylethylamine (173 mg, 1.34 mmol), dry Methanol (4 mL) then ferrous cyclopenta-2,4-dien-1-yl(diphenyl)phosphane dichloropalladium (24.5 mg, 0.034 mmol). The reaction was degassed and filled with CO (gas). The sealed vessel was heated at 70° C. for 18 hrs. The vessel was cooled to rt and purged with N₂. Volatiles were removed in vacuo and the residue was purified by flash chromatography (0-50% EtOAc in iso-hexanes) to afford the title compound (34 mg, 27% yield) as a yellow oil.

[M+H]⁺=181.2

1H NMR (DMSO, 400 MHz) d 1.26 (7H, d, J=6.9 Hz), 3.14 (1H, p, J=6.9 Hz), 3.92 (3H, s), 7.94 (1H, dd, J=1.4, 0.4 Hz), 9.26 (1H, d, J=1.3 Hz).

### Methyl 2-isopropylpyrimidine-5-carboxylate

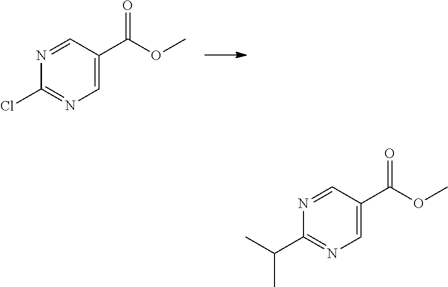

To a microwave vial was added THF (8 mL), chloro(isopropyl)magnesium (1.3 mL, 2.61 mmol) and zinc chloride (1.6 mL, 3.04 mmol). The solution was stirred at rt for 10 mins before methyl 2-chloropyrimidine-5-carboxylate (300 mg, 1.74 mmol) then tetrakis(triphenylphosphine)palladium(0) (60.3 mg, 0.052 mmol) were added. The reaction was heated in a microwave 80° C. for 60 min. The reaction mixture was partitioned between EtOAc (10 mL) and sat. NH₄Cl (10 mL). The aqueous layer was extracted with EtOAc (2×10 mL). Combined organic layers were washed with water (10 mL) and brine (10 mL) then dried (MgSO₄), filtered and concentrated in vacuo. Purification by flash chromatography (10% EtOAc in iso-hexanes) afforded the title compound (106 mg, 33% yield) as a colourless oil.

[M+H]⁺=181.3

### [1,2,4]triazolo[1,5-a]pyrimidine-5-carboxylic acid

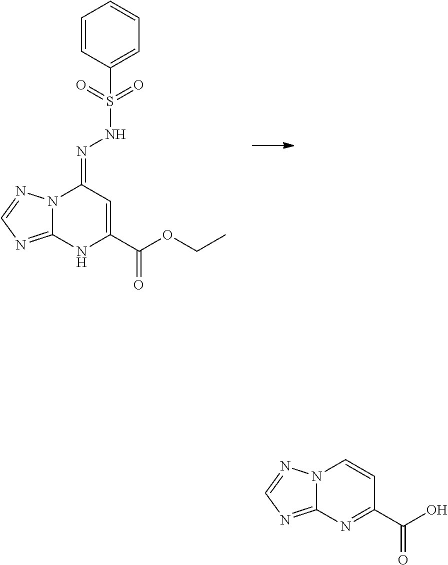 [SMILES: CCOC(=O)c1c/c(=N\NS(=O)(=O)c2ccccc2)n2ncnc2[nH]1 ; O=C(O)c1ccn2ncnc2n1]

A solution of lithium hydroxide (66.7 mg, 2.79 mmol) in water (10 mL) was treated with ethyl (7E)-7-(p-tolylsulfonylhydrazono)-4H-[1,2,4]triazolo[1,5-a]pyrimidine-5-carboxylate hydrochloride (115 mg, 0.279 mmol) and the mixture heated to 100° C. for 2 hrs. The mixture was cooled and partitioned over EtOAc (10 mL). The aqueous was extracted with further EtOAc (2×10 mL) then and adjusted to pH 2 with 1M HCl. The aqueous was then extracted with EtOAc (20×7.5 mL) and the combined organics dried (MgSO₄), filtered and concentrated to afford the title compound (66 mg, 49% yield)

[M+H]⁺=165.1

### Ethyl (7E)-7-(p-tolylsulfonylhydrazono)-4H-[1,2,4]triazolo[1,5-a]pyrimidine-5-carboxylate Hydrochloride

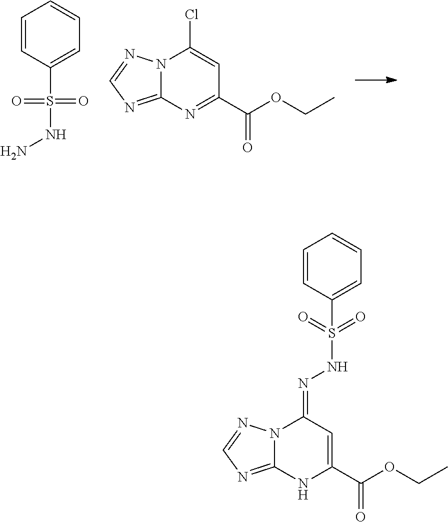

A mixture of ethyl 7-chloro-[1,2,4]triazolo[1,5-a]pyrimidine-5-carboxylate (30 mg, 0.132 mmol) and 4-methylbenzenesulfonohydrazide (24.7 mg, 0.132 mmol) in anhydrous DCM (0.5 mL) were stirred for 18 hrs at rt. The precipitate was filtered, washing with DCM (50 mL). The filtrate was concentrated to afford the title compound (35 mg, 61% yield).

[M+H]⁺=377.4

NMR (DMSO) δ: 1.36 (3H, t, J=7.1 Hz), 2.40 (3H, s), 4.39 (2H, q, J=7.1 Hz), 7.08 (1H, s), 7.42 (2H, d, J=8.5 Hz), 7.72 (2H, d, J=8.3 Hz), 8.67 (1H, s), 10.63 (1H, s), 11.14 (1H, s)

### Ethyl 7-chloro-[1,2,4]triazolo[1,5-a]pyrimidine-5-carboxylate

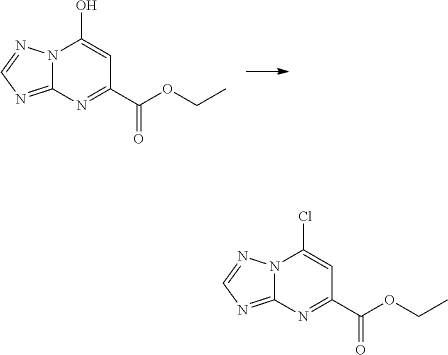

A mixture of ethyl 7-hydroxy-[1,2,4]triazolo[1,5-a]pyrimidine-5-carboxylate (285 mg, 1.37 mmol) and phosphorus oxychloride (2.55 mL, 27.4 mmol) were heated together at 90° C. for 2 hrs. On cooling, the mixture was concentrated under vacuum. The mixture was azeotroped with toluene (5 mL). The crude product was purified by flash chromatography (0 to 5% MeOH/DCM) to afford the title compound (88 mg, 28% yield) as a white powder.

[M+H]⁺=227.3/229.3

### Ethyl 7-hydroxy-[1,2,4]triazolo[1,5-a]pyrimidine-5-carboxylate

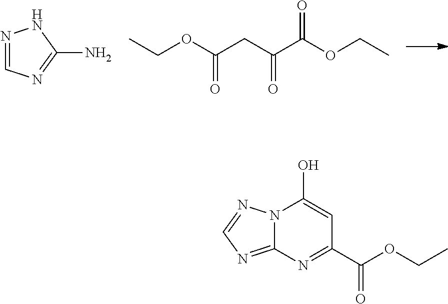

A solution of 1H-1,2,4-triazol-5-amine (0.54 g, 6.42 mmol) and diethyl 2-oxobutanedioate (1.33 g, 7.06 mmol) in acetic acid (10 mL) was heated to 90° C. for 18 hrs. Upon cooling to rt the volatiles were removed in vacuo and purified by flash chromatography (0-5% (1% AcOH in MeOH) in DCM). The title compound was isolated (0.67 g, 43% yield) as a yellow solid.

[M+H]⁺=209.2

### N-isopropyl-N-methyl-D-alanine

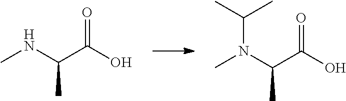

N-Methyl-D-alanine (1.0 g, 9.7 mmol) was dissolved in methanol (100 mL) to which acetone (5.63 g, 96.98 mmol) was added. 10% Pd/C (500 mg) was added. The reaction mixture was shaken on a Parr hydrogenator at 10 psi for 18 hrs after which time the catalyst was filtered off through celite and the residue washed with methanol (200 mL) and water (20 mL). The combined filtrates were evaporated in vacuo to give a white solid. The product was recrystallised from MeOH/diethyl ether to give a white solid identified as the title compound (1.39 g, 98% yield)

[M+H]⁺=146.24

### (R)-1-isopropylpyrrolidine-2-carboxylic Acid

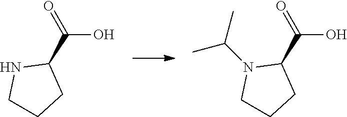

To a solution of (R)-pyrrolidine-2-carboxylic acid (14.8 g, 129 mmol) in MeOH (0.75 L) and acetone (12.74 mL, 174 mmol) was added a slurry of Pd—C(10% Pd/C with 50% water) (2.95 g, 1.39 mmol) in EtOH (10 mL). The resulting suspension was stirred at rt under H₂ (2 bar) for 18 hrs. The reaction was then filtered through celite, washing with MeOH (2×200 mL). The resulting solution was concentrated in vacuo to afford a yellow solid. This was dissolved in MeOH (30 mL) and precipitated with diethyl ether (300 mL). The resulting white solid was collected by filtration to afford the title compound (18.14 g, 88% yield) as a white solid.

### (R)-2-((tert-butoxycarbonyl)(methyl)amino)-4-phenylbutanoic Acid

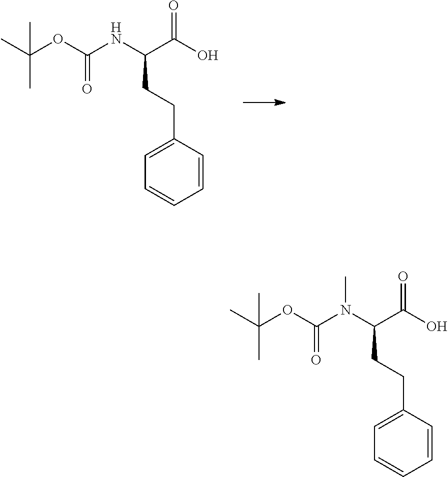 [SMILES: CC(C)(C)OC(=O)N[C@H](CCc1ccccc1)C(=O)O ; CN(C(=O)OC(C)(C)C)[C@H](CCc1ccccc1)C(=O)O]

To BOC-D-HomoPhe-OH (1 g, 3.58 mmol) in THF (25 mL) and DMF (5 mL), cooled in an ice bath to 0° C., was added sodium hydride (1.28 g, 32.22 mmol). The mixture was stirred in an ice bath for 60 mins. To the cooled reaction was added iodomethane (0.27 mL, 4.30 mmol). The mixture was stirred and allowed to warm to rt with ice bath in place and left stirring overnight. The reaction was cooled, quenched with water (40 mL) and concentrated, acidified with citric acid to pH 1.5 and extracted with DCM (5×35 mL). The organics were concentrated to afford a pale yellow oil. Crude product was purified by flash chromatography (40% EtOAc in Pet. Ether) to afford the title compound as a white solid (683 mg, 65% yield).

[M+Na]⁺ 316.0

### Methyl (R)-2-amino-4-phenylbutanoate

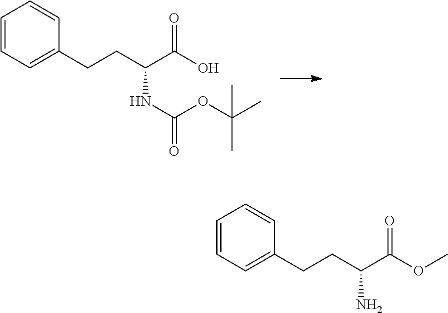 [SMILES: CC(C)(C)OC(=O)N[C@H](CCc1ccccc1)C(=O)O ; COC(=O)[C@H](N)CCc1ccccc1]

(R)-2-((tert-butoxycarbonyl)amino)-4-phenylbutanoic acid (320 mg, 1.09 mmol) was reacted following general method A to afford the title compound as a yellow solid as a hydrochloride salt (253 mg, 96% yield).

[M+H]⁺=194.4

### Methyl (R)-4-phenyl-2-(pyrrolidin-1-yl)butanoate

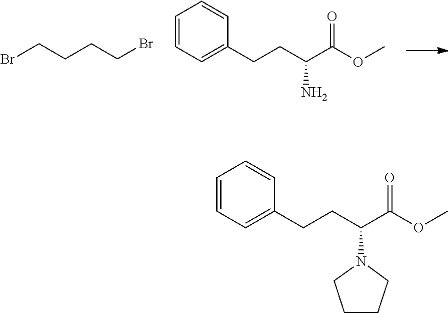

To a stirred solution of 1,4-dibromobutane (156 μL, 1.31 mmol) and methyl (R)-2-amino-4-phenylbutanoate (250 mg, 1.09 mmol) in acetonitrile (20 mL) was added K₂CO₃ (451 mg, 3.27 mmol) and the reaction was stirred at 80° C. for 3 days. The reaction mixture was diluted with EtOAc (50 mL) and washed with sat. NaHCO₃ (25 mL), water (25 mL), brine (25 mL), dried (MgSO4), filtered and concentrated. Purification via flash chromatography (0-60% EtOAc in Pet. Ether) afforded the title compound as a colourless oil (110 mg, 41% yield).

[M+H]⁺=248.3

### Methyl (S)-3-(3,4-difluorophenyl)-2-(2-((2R,6S)-2,6-dimethylpiperidin-1-yl)acetamido) propanoate

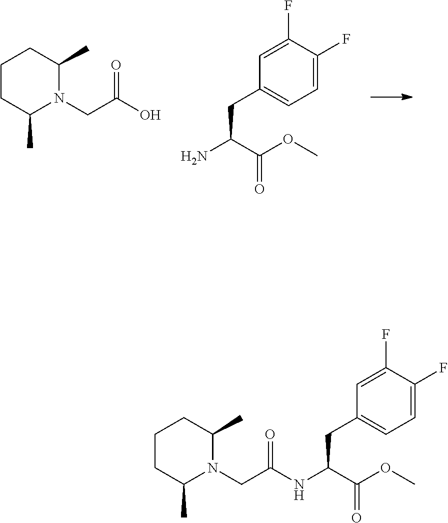 [SMILES: C[C@@H]1CCC[C@H](C)N1CC(=O)O ; COC(=O)[C@@H](N)Cc1ccc(F)c(F)c1 ; COC(=O)[C@H](Cc1ccc(F)c(F)c1)NC(=O)CN1[C@H](C)CCC[C@@H]1C]

Following general method C(ii), [(2R,6S)-2,6-Dimethylpiperidine-1-yl]acetic acid (561 mg, 3.28 mmol) was reacted with methyl (S)-2-amino-3-(3,4-difluorophenyl)propanoate (750 mg, 2.98 mmol). Flash chromatography (0-8% MeOH in CHCl₃) afforded the title compound as a white solid (1.05 g, 96% yield)

[M+H]⁺=369.2

### (S)-3-(3,4-difluorophenyl)-2-(2-((2R,6S)-2,6-dimethylpiperidin-1-yl)acetamido)propanoic Acid

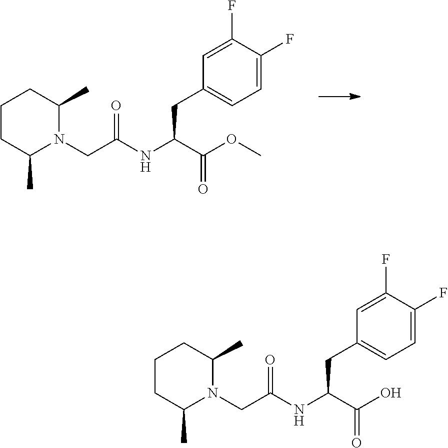 [SMILES: COC(=O)[C@H](Cc1ccc(F)c(F)c1)NC(=O)CN1[C@H](C)CCC[C@@H]1C ; C[C@@H]1CCC[C@H](C)N1CC(=O)N[C@@H](Cc1ccc(F)c(F)c1)C(=O)O]

Methyl (S)-3-(3,4-difluorophenyl)-2-(2-((2R,6S)-2,6-dimethylpiperidin-1-yl)acetamido) propanoate (1.05 g, 2.85 mmol) was reacted following general method D, to afford the title compound as a white solid, (844 mg, 84% yield).

[M+H]⁺=355.3

### Methyl (R)-2-((tert-butoxycarbonyl)amino)-6-(piperidin-1-yl)hexanoate

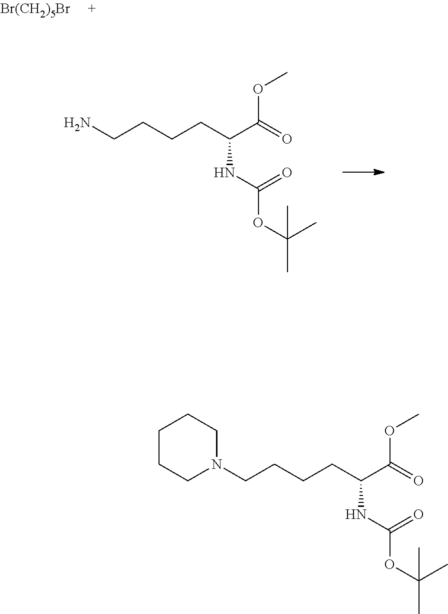 [SMILES: "Br(CH2)5Br" ; COC(=O)[C@@H](CCCCN)NC(=O)OC(C)(C)C ; COC(=O)[C@@H](CCCCN1CCCCC1)NC(=O)OC(C)(C)C]

Methyl (tert-butoxycarbonyl)-D-lysinate (1.82 g, 6.99 mmol) was dissolved in acetonitrile (150 mL), 1,5-dibromopentane (1.69 g, 7.34 mmol) was added followed by K₂CO₃ (2.89 g, 20.9 mmol) and the reaction was stirred at 60° C. for 3 hrs. Reaction mixture was cooled to rt and concentrated. The residue was taken up in CHCl₃ (75 mL) and washed with Na₂CO₃ (35 mL) and brine (35 mL). The organic layer was then dried over Na₂SO₄ filtered and evaporated. The crude product was purified by flash chromatography 0-10% (1% NH₃ in MeOH) in DCM visualising product on TLC by Ninhydrin staining. The title compound was isolated as a pale yellow oil (1.372 g, 60% yield)

[M+H]⁺=329.4

### (R)-2-((tert-butoxycarbonyl)amino)-6-hydroxyhexanoic Acid

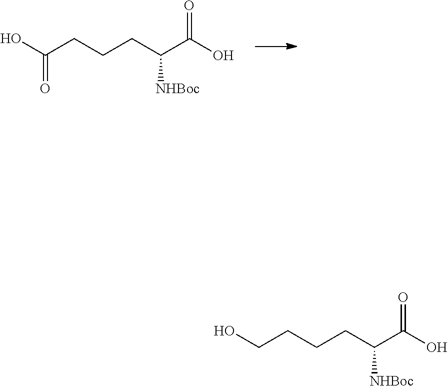 [SMILES: CC(C)(C)OC(=O)N[C@H](CCCC(=O)O)C(=O)O ; CC(C)(C)OC(=O)N[C@H](CCCCO)C(=O)O]

A solution of (R)-2-((tert-butoxycarbonyl)amino)hexanedioic acid (2.00 g, 7.65 mmol) in anhydrous THF (80 mL) under nitrogen was cooled to D° C. Sodium borohydride (0.87 mg, 22.96 mmol) was added followed by dropwise addition of a solution of Iodine (2.53 g, 9.95 mmol) in THF (15 mL) over 20 min.

The ice-bath was removed and the mixture allowed to warm to rt over 60 min. On completion the reaction was cooled to 0° C. and quenched by the dropwise addition of methanol (15 mL). The mixture was diluted with EtOAc (100 mL) and washed with water (3×50 mL). The combined aqueous extracts were acidified to pH 3 with 1M HCl and extracted with 2-methyl tetrahydrofuran (3×50 mL). The combine organic extracts were dried (Na₂SO₄), filtered and concentrated. The title compound was isolated (1.41 g, 75% yield) as a pale yellow foam.

### (R)-5-((tert-butoxycarbonyl)amino)-6-((S)-2-(((3-chloro-1H-indol-5-yl)methyl)carbamoyl)azetidin-1-yl)-6-oxohexyl Methanesulfonate

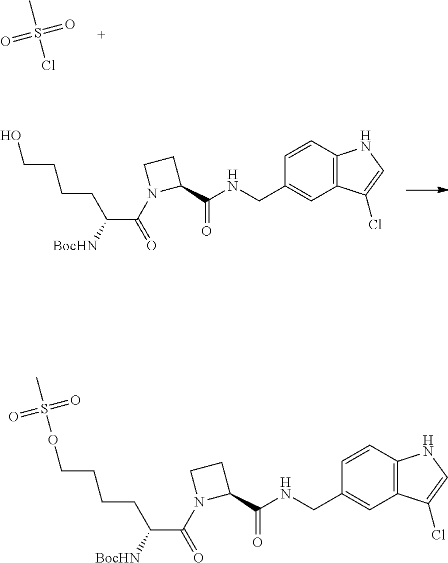

A solution of tert-butyl ((R)-1-((S)-2-(((3-chloro-1H-indol-5-yl)methyl)carbamoyl)azetidin-1-yl)-6-hydroxy-1-oxohexan-2-yl)carbamate (400 mg, 0.81 mmol) and triethylamine (0.14 mL, 0.97 mmol) in anhydrous DCM (5 mL) was cooled to 0° C. To this was added dropwise methane sulfonylchloride (75 μL, 0.97 mmol). On completion of the addition the ice bath was removed and the mixture stirred at rt for 18 hrs. The reaction mixture was diluted with DCM (20 mL) and washed with NaHCO₃ aq (20 mL), then brine (20 mL), dried over MgSO₄, filtered and concentrated. The crude product was purified by flash chromatography (0-5% MeOH in DCM) to afford the title compound (386 mg, 83% yield) as a colourless oil.

LCMS [M+]=570.9/572

### tert-butyl ((R)-1-((S)-2-(((3-chloro-1H-indol-5-yl)methyl)carbamoyl)azetidin-1-yl)-6-morpholino-1-oxohexan-2-yl)carbamate

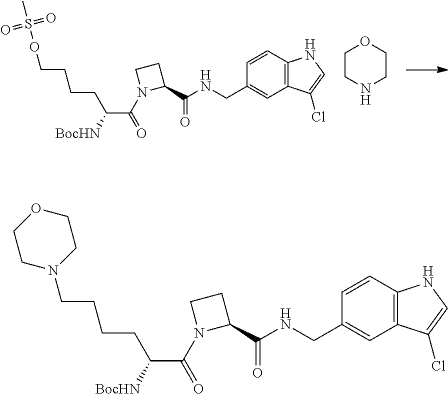

(R)-5-((tert-butoxycarbonyl)amino)-6-((S)-2-(((3-chloro-1H-indol-5-yl)methyl)carbamoyl)azetidin-1-yl)-6-oxohexyl methanesulfonate (190 mg, 0.33 mmol) was dissolved in acetonitrile (15 mL). Morpholine (44 μL, 0.50 mmol) was added followed by triethylamine (139 IL, 1.00 mmol) and the reaction was stirred at reflux for 18 hrs. The reaction mixture was concentrated, taken up in CHCl₃ and washed with sat. aq. Na₂CO₃ and brine. The organic layer was then dried over Na₂SO₄, filtered and evaporated. The crude was purified by flash chromatography (0-8% methanol in DCM) to afford the title product as a colourless oil (111 mg, 69% yield).

LCMS [M]⁺=561.7

### (S)-1-(2,2,2-trifluoroacetyl)azetidine-2-carboxylic Acid

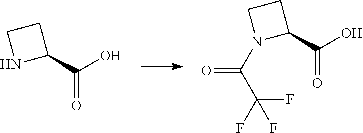 [SMILES: O=C(O)[C@@H]1CCN1 ; O=C(O)[C@@H]1CCN1C(=O)C(F)(F)F]

To a solution of (S)-azetidine-2-carboxylic acid (350 mg, 3.46 mmol) in dry DMF (7.5 mL) was added diisopropylamine (0.97 mL, 6.92 mmol) followed by ethyl trifluoroacetate (2.1 mL, 17.31 mmol). The reaction mixture was heated to 40° C. for 18 hrs. The solution was concentrated in vacuo to afford the title compound (682 mg, 100% yield).

### (S)—N-(quinolin-8-yl)-1-(2,2,2-trifluoroacetyl)azetidine-2-carboxamide

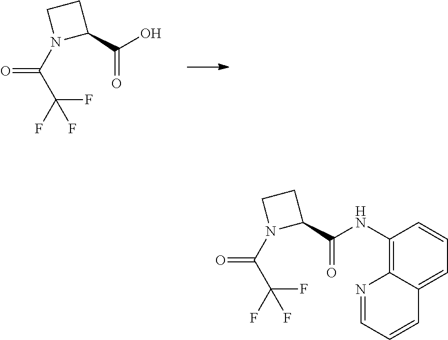 [SMILES: O=C(O)[C@@H]1CCN1C(=O)C(F)(F)F ; O=C(Nc1cccc2cccnc12)[C@@H]1CCN1C(=O)C(F)(F)F]

Following general method C(i), crude (S)-1-(2,2,2-trifluoroacetyl)azetidine-2-carboxylic acid (682 g, 3.46 mmol) was reacted with 8-aminoquinoline (499 mg, 3.46 mmol). The title compound was isolated as a yellow oil (890 mg, 80% yield, 99.5% ee)

[M+H]⁺=324.0

### (2S,3R)-3-(3,4-difluorophenyl)-N-(quinolin-8-yl)azetidine-2-carboxamide

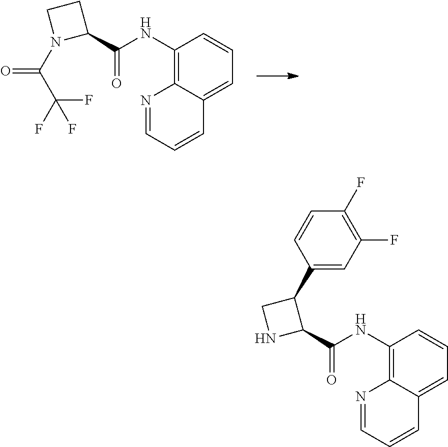 [SMILES: O=C(Nc1cccc2cccnc12)[C@@H]1CCN1C(=O)C(F)(F)F ; O=C(Nc1cccc2cccnc12)[C@H]1NC[C@H]1c1ccc(F)c(F)c1]

A solution of (S)—N-(quinolin-8-yl)-1-(2,2,2-trifluoroacetyl)azetidine-2-carboxamide (674 mg, 2.08 mmol), 3,4-difluoroiodobenzene (0.75 mL, 6.25 mmol), silver acetate (696 mg, 4.17 mmol), palladium acetate (47 mg, 0.208 mmol), and dibenzyl phosphate (116 mg, 0.417 mmol) in dry 1,2-dichloroethane (3 mL) under argon was heated at 110° C. for 24 hrs. The reaction mixture was cooled to rt and treated with 7M NH₃ in MeOH (31.3 mL, 218.9 mmol) for 2 hrs. The reaction mixture was concentrated in vacuo and purified by flash chromatography (10-100% EtOAc in cyclohexane) to afford the title compound (178 mg, 25% yield).

[M+H]⁺=340.0

### (2S,3R)-1-(tert-butoxycarbonyl)-3-(3,4-difluorophenyl)azetidine-2-carboxylic Acid

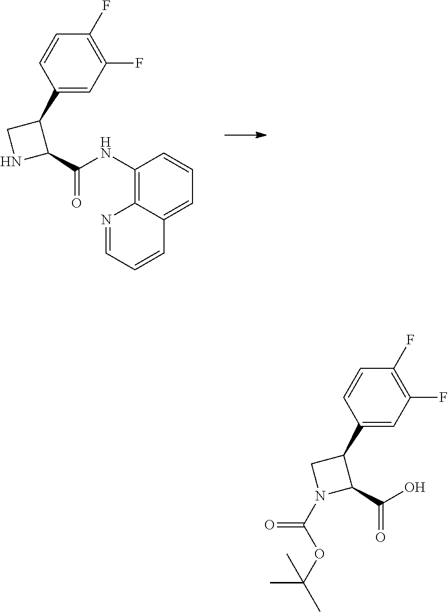 [SMILES: O=C(Nc1cccc2cccnc12)[C@H]1NC[C@H]1c1ccc(F)c(F)c1 ; CC(C)(C)OC(=O)N1C[C@@H](c2ccc(F)c(F)c2)[C@H]1C(=O)O]

To a solution of (2S,3R)-3-(3,4-difluorophenyl)-N-(quinolin-8-yl)azetidine-2-carboxamide (178 mg, 0.53 mmol) in dry acetonitrile (4 mL) was added Boc anhydride (343 mg, 1.57 mmol). The solution was heated at 50° C. for 15 min before 4-(dimethylamino)pyridine (6.4 mg, 0.053 mmol) was added and the reaction continued at 50° C. for 2 hrs. The reaction mixture was concentrated in vacuo and the residue was dissolved in THF (4 mL) and water (2 mL). After cooling to OC, 30% aqueous hydrogen peroxide solution (0.16 mL, 5.25 mmol) was added followed by lithium hydroxide (132 mg, 3.15 mmol). The reaction was warmed to rt the heated at 50° C. for 18 hrs. After cooling to rt, the solution was diluted with EtOAc (20 mL) and sodium sulfite (aq) (20 mL) was added. After stirring for 15 min, the layers were separated and the aqueous layer was acidified with 1M HCl to pH 3 and washed with EtOAc (4×20 mL). The combined organic extracts were dried (Na₂SO₄), filtered and concentrated to afford the title compound (79 mg, 48% yield) as a white solid.

[M-boc+H]⁺=214.0

### tert-Butyl ((4-aminofuro[3,2-c]pyridin-2-yl)methyl)carbamate

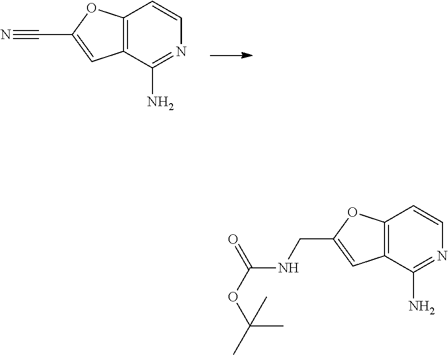 [SMILES: N#Cc1cc2c(N)nccc2o1 ; CC(C)(C)OC(=O)NCc1cc2c(N)nccc2o1]

Nickel(II) chloride hexahydrate (0.608 g, 2.56 mmol), di-tert-butyl dicarbonate (1.15 g, 5.27 mmol), and 4-aminofuro[3,2-c]pyridine-2-carbonitrile hydrochloride (500 mg, 2.56 mmol) were dissolved in MeOH (14 mL) and THE (7 mL). The solution was cooled to 0° C. in an ice-water bath and sodium borohydride (0.677 g, 17.89 mmol) was added portion wise. The reaction warmed to rt and stirred for 18 hrs. The reaction was quenched with water (0.5 mL) and the mixture was filtered through a plug of cotton wool and washed with THE (3×10 mL). The crude product purified by flash chromatography 0-10% (1% NH₃ in MeOH) in DCM. The title compound (273 mg, 38% yield) was isolated as a pale white solid.

[M+H]⁺=264.3

1H NMR (500 MHz, DMSO-d6) δ: 1.40 (s, 9H), 4.22 (d, J=6.0 Hz, 2H), 6.39 (s, 2H), 6.73 (dd, J=5.9, 1.0 Hz, 1H), 6.76 (s, 1H), 7.48 (s, 1H), 7.72 (d, J=5.8 Hz, 1H).

### tert-Butyl ((5-Chlorobenzo[b]thiophen-2-yl)methyl)carbamate

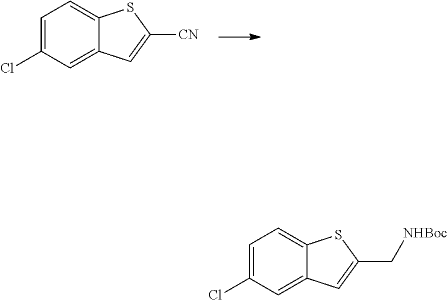 [SMILES: N#Cc1cc2cc(Cl)ccc2s1 ; CC(C)(C)OC(=O)NCc1cc2cc(Cl)ccc2s1]

5-Chlorobenzo[b]thiophene-2-carbonitrile (250 mg, 1.291 mmol), di-tert-butyl dicarbonate (564 mg, 2.58 mmol) and nickel(II) chloride hexahydrate (34 mg, 0.142 mmol) were dissolved in MeOH (25 mL). The solution was cooled to O ° C. and sodium borohydride (342 mg, 9.04 mmol) was added portion wise. The reaction was allowed to warm to rt and stirred for 18 hrs. The reaction was quenched with water (1 mL) and filtered through a plug of cotton wool and concentrated in vacuo. The crude was purified by flash chromatography (0-20% EtOAc/isohexane) to afford the title compound (214 mg, 50% yield) as a white solid.

No ionisation

1H NMR (500 MHz, DMSO-d6) δ 1.41 (s, 9H), 4.38 (d, J=6.2 Hz, 2H), 7.22 (s, 1H), 7.33 (dd, J=8.6, 2.2 Hz, 1H), 7.63 (t, J=6.1 Hz, 1H), 7.88 (d, J=2.1 Hz, 1H), 7.95 (d, J=8.6 Hz, 1H).

### Synthesis of (7-Chloro-3,4-dihydro-2H-benzo[b][1,4]oxazin-2-yl)methanamine

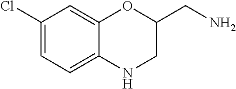 [SMILES: NCC1CNc2ccc(Cl)cc2O1]

### Ethyl 7-chloro-3,4-dihydro-2H-benzo[b][1,4]oxazine-2-carboxylate

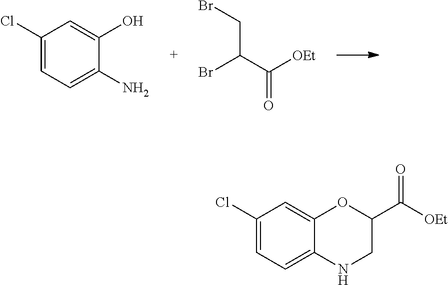 [SMILES: Nc1ccc(Cl)cc1O ; CCOC(=O)C(Br)CBr ; CCOC(=O)C1CNc2ccc(Cl)cc2O1]

To a solution of ethyl 2,3-dibromopropanoate (1 ml, 6.88 mmol) in acetone (25 mL) was added K₂CO3 (0.96 g, 6.95 mmol) and ethyl 2,3-dibromopropanoate (1 ml, 6.88 mmol). The reaction was left stirring at 60° C. 18 hrs. The precipitates were filtered off and the solvent was concentrated in vacuo. The residue was dissolved in cold 1N sodium hydroxide (30 mL) and extracted with EtOAc (3×30 mL). The combined organic layers were washed with water (30 mL) and dried over Na₂SO₄, filtered, and concentrated in vacuo. Flash chromatography (0-50% EtOAc/isohexane) afforded the title compound (1.21 g, 68% yield) as a dark brown solid.

[M+H]⁺=242.2

1H NMR (500 MHz, DMSO-d6) δ 1.18 (t, J=7.1 Hz, 3H), 3.35-3.49 (m, 2H), 4.09-4.21 (m, 2H), 4.95-5.04 (m, 1H), 6.02 (s, 1H), 6.57 (d, J=8.5 Hz, 1H), 6.72 (dd, J=8.4, 2.4 Hz, 1H), 6.80 (d, J=2.4 Hz, 1H).

### 7-Chloro-3,4-dihydro-2H-benzo[b][1,4]oxazine-2-carboxamide

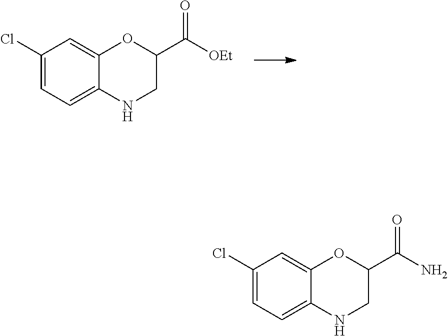 [SMILES: CCOC(=O)C1CNc2ccc(Cl)cc2O1 ; NC(=O)C1CNc2ccc(Cl)cc2O1]

Ethyl 7-chloro-3,4-dihydro-2H-benzo[b][1,4]oxazine-2-carboxylate (1.2 g, 4.97 mmol) and ammonium hydroxide, 28% in water (10 mL, 71.9 mmol) were mixed in a microwave tube. The mixture was heated to 70° C. for 30 min in a microwave reactor. The reaction was cooled to rt and partitioned in water (20 mL) and EtOAc (20 mL). The organic layer was separated and the aqueous layer was extracted with EtOAc (2×20 mL). The combined organic extracts were washed with NaHCO₃ (sat., aq., 10 mL), dried over Na₂SO₄, filtered and concentrated in vacuo to afford the title compound (760 mg, 70% yield) as a brown solid.

[M+H]⁺=213.3

1H NMR (500 MHz, DMSO-d6) δ 3.22-3.28 (m, 1H), 3.35-3.44 (m, 1H), 4.44-4.57 (m, 1H), 6.02 (t, J=2.7 Hz, 1H), 6.55-6.61 (m, 1H), 6.73 (dd, J=8.4, 2.4 Hz, 1H), 6.82 (d, J=2.4 Hz, 1H), 7.42 (s, 2H).

### (7-Chloro-3,4-dihydro-2H-benzo[b][1,4]oxazin-2-yl)methanamine

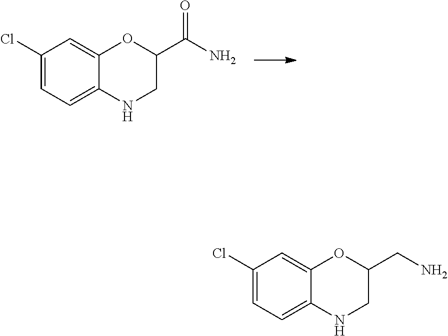 [SMILES: NC(=O)C1CNc2ccc(Cl)cc2O1 ; NCC1CNc2ccc(Cl)cc2O1]

To a solution of 7-chloro-3,4-dihydro-2H-benzo[b][1,4]oxazine-2-carboxamide (750 mg, 3.53 mmol) in THF (30 mL) at 0° C. was added LiAlH₄, 2M in THF (3.5 mL, 7.00 mmol). The reaction was allowed to warm to rt and then heated at 45° C. for 20 hrs. On completion, the reaction was cooled to 0° C., water (0.3 mL), NaOH (15 wt % aq. solution, 0.3 mL) and water (0.9 mL) were added dropwise sequentially. The resulting suspension was filtered and the solid was further washed with EtOAc (3×20 mL). The combined organic layer was dried on Na₂SO₄, filtered and concentrated in vacuo. Reverse phase flash chromatography (0-50% MeCN in 10 mM Ammonium Bicarbonate) afforded the title compound (245 mg, 32% yield) as a brown oil.

[M+H]⁺=199.2

1H NMR (500 MHz, DMSO-d6) δ 1.54 (s, 2H), 2.61-2.71 (m, 1H), 2.72-2.81 (m, 1H), 2.95-3.03 (m, 1H), 3.34-3.38 (m, 1H), 3.81-3.91 (m, 1H), 5.91 (s, 1H), 6.51-6.58 (m, 1H), 6.65-6.72 (m, 2H)

### Synthesis of 2-(Aminomethyl)thieno[3,2-c]pyridin-4-amine

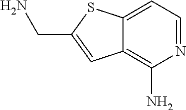 [SMILES: NCc1cc2c(N)nccc2s1]

### 4-Phenoxythieno[3,2-c]pyridine

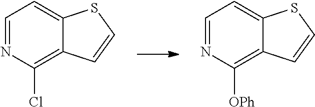 [SMILES: Clc1nccc2sccc12 ; c1ccc(Oc2nccc3sccc23)cc1]

A mixture of 4-chlorothieno[3,2-c]pyridine (10 g, 59.0 mmol) and phenol (36.6 g, 389 mmol) was warmed to 45° C. to form a homogeneous solution. KOH (5.6 g, 100 mmol) was added and the reaction was heated to 140° C. for 18 hrs. The reaction mixture was cooled to 50° C. and diluted with 2N NaOH (250 mL), before being further cooled to rt, extracted with DCM (3×400 mL) and washed with brine (100 mL). The combined organic layer was dried (MgSO₄), filtered and concentrate in vacuo to afford 4-phenoxythieno[3,2-c]pyridine (13.25 g, 92% yield) as an dark brown crystalline solid.

[M+H]⁺=228.2

1H NMR (500 MHz, DMSO-d6) δ 7.21-7.28 (m, 3H), 7.45 (dd, J=8.4, 7.3 Hz, 2H), 7.67 (d, J=5.5 Hz, 1H), 7.80 (d, J=5.6 Hz, 1H), 7.92 (dd, J=5.5, 4.3 Hz, 2H).

### Thieno[3,2-c]pyridin-4-amine

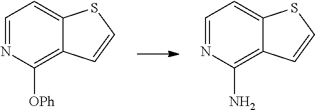 [SMILES: c1ccc(Oc2nccc3sccc23)cc1 ; Nc1nccc2sccc12]

4-phenoxythieno[3,2-c]pyridine (13.2 g, 58.1 mmol) and ammonium acetate (70 g, 908 mmol) were mixed and heated to 150° C. After 16 further ammonium acetate (35 g, 454 mmol) was added. After 72 hrs the reaction mixture was cooled to 50° C. and quenched with 2M NaOH (200 mL). The aqueous phase was then allowed to cool to rt and extracted with EtOAc (3×200 mL). The combined organic extracts were washed with brine (200 mL), dried (MgSO₄), filtered and concentrated in vacuo. The crude product was sonicated with 2M NaOH (100 mL). EtOAc (100 mL) was added and the organic layer was separated. The aqueous layer was extracted with 3×100 mL EtOAc. The combined organics were washed with 100 mL of brine, dried on MgSO₄, filtered and concentrated in vacuo, to afford thieno[3,2-c]pyridin-4-amine (5.6 g, 63% yield) as a dark brown solid.

1H NMR (500 MHz, DMSO-d6) δ 6.54 (s, 2H), 7.11-7.14 (m, 1H), 7.56 (d, J=5.5 Hz, 1H), 7.63-7.67 (m, 1H), 7.75 (d, J=5.7 Hz, 1H).

### N-(thieno[3,2-c]pyridin-4-yl)benzamide

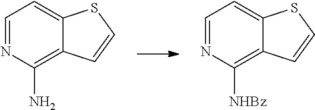

To a solution of thieno[3,2-c]pyridin-4-amine (5.6 g, 37.3 mmol) in pyridine (60 mL) was added benzoic anhydride (9.28 g, 41.0 mmol) at rt. The mixture was heated to 125° C. After 2 hrs the reaction was cooled to rt then concentrated in vacuo. The crude product mixture was partitioned between water (200 mL) and DCM (200 mL). The organic layer was separated and the aqueous layer extracted with DCM (2×200 mL). The combined organics were washed with brine (100 mL), dried (MgSO₄), filtered and concentrated in vacuo. The crude product was purified by flash chromatography (5% to 100% EtOAc in isohexane) to afford a thick yellow solid. The product was partitioned in DCM (100 mL) and Na₂CO₃ solution (aq., sat., 100 mL). The mixture was sonicated for 5 mins. The organic layer was separated and the aqueous layer was extracted with DCM (2×100 mL). The combined organic extract was dried (Na₂SO₄), filtered and concentrated in vacuo to afford N-(thieno[3,2-c]pyridin-4-yl)benzamide (6.62 g, 69% yield) as a foaming yellow solid.

[M+H]⁺=255.2

### N-(2-formylthieno[3,2-c]pyridin-4-yl)benzamide

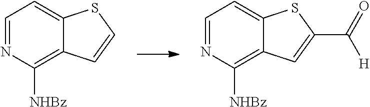

To a solution of N-(thieno[3,2-c]pyridin-4-yl)benzamide (6.6 g, 26.0 mmol) in THF (120 mL) at −78° C. was added LDA, 2M in THF/heptane/ethylbenzene (28.5 mL, 57.1 mmol) dropwise (internal temperature <−70° C.). After addition, the reaction mixture was stirred at −78° C. for 45 mins. DMF (7 mL, 90 mmol) was added dropwise then the cooling bath was removed to allow the reaction to warm to rt and stirred overnight. It was quenched with NH₄Cl (sat., aq., 100 mL). The aqueous layer was extracted with EtOAc (5×100 mL). The combined organic extracts were dried (Na₂SO₄), filtered and concentrated in vacuo. The crude product was purified by flash chromatography (5-100% THF in iso-hexane) to afford the title compound (4.62 g, 61% yield) as a pale yellow solid.

[M+H]⁺=283.2

### N-(2-(((2,4-dimethoxybenzyl)amino)methyl)thieno[3,2-c]pyridin-4-yl)benzamide)

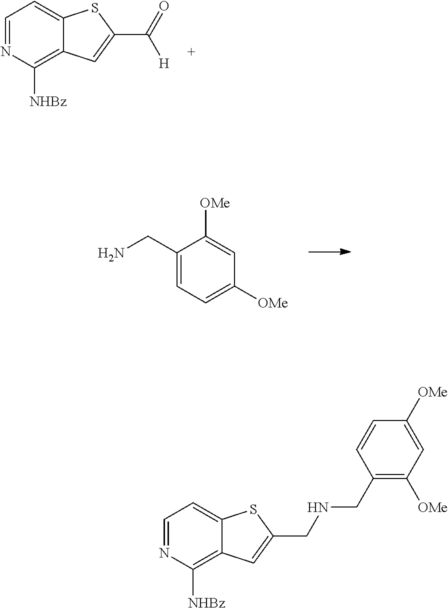

N-(2-formylthieno[3,2-c]pyridin-4-yl)benzamide (4.6 g, 16.29 mmol) and (2,4-dimethoxyphenyl)methanamine (3.27 g, 19.55 mmol) were mixed with AcOH (0.94 mL) and THF (110 mL). After 3 hrs, sodium triacetoxyborohydride (5.18 g, 24.44 mmol) was added. The reaction was stirred at rt for 3 hrs and then heated to 40° C. overnight. The reaction was quenched with NaHCO₃ (sat., aq., 100 mL). The organic layer was separated and the aqueous layer was extracted with EtOAc (3×100 mL). The combined organics were dried (Na₂SO₄), filtered and concentrated in vacuo. The crude product was purified by flash chromatography (0-100% EtOAc in iso-hexane) to afford the title compound (3.9 g, 49% yield) as a pale yellow solid.

### 2-(Aminomethyl)thieno[3,2-c]pyridin-4-amine

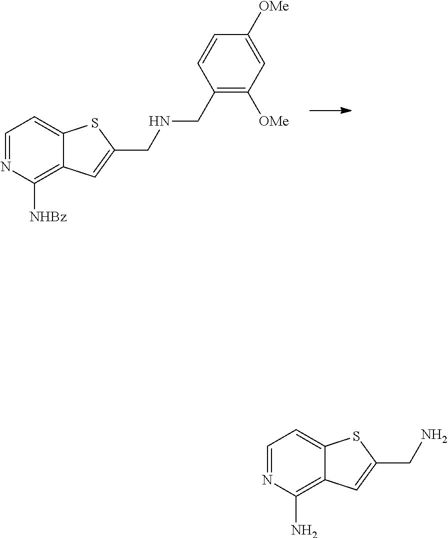

To a solution of N-(2-(((2,4-dimethoxybenzyl)amino)methyl)thieno[3,2-c]pyridin-4-yl)benzamide (650 mg, 1.5 mmol) in AcOH (6 mL) was added HCl (37 wt %, aq., 9 mL). The solution was heated to 100° C. The reaction cooled to rt and the solvent and excess acid were removed in vacuo. The reaction mixture was partitioned between 2M NaOH (aq. 150 mL) and EtOAc (150 mL). The aqueous phase was extracted with THF (5×200 mL). The combined organic extract was dried (Na₂SO₄), filtered and concentrated in vacuo to afford a dark red solid. The crude product was purified by reverse phase flash chromatography (0-50% MeCN in 10 mM Ammonium Bicarbonate) to afford the title compound (770 mg, 47% yield) as a pale red solid.

[M+H]⁺=180.2

1H NMR (500 MHz, DMSO-d6) δ 2.02 (s, 2H), 3.96 (d, J=1.3 Hz, 2H), 6.36 (s, 2H), 7.03 (d, J=5.7 Hz, 1H), 7.38-7.42 (m, 1H), 7.69 (d, J=5.6 Hz, 1H).

### Synthesis of Ethyl 1-(2-(aminomethyl)-3-fluoro-4-methoxyphenyl)-1H-pyrazole-3-carboxylate

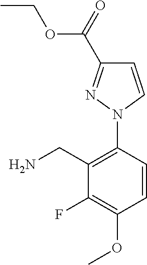 [SMILES: CCOC(=O)c1ccn(-c2ccc(OC)c(F)c2CN)n1]

### Ethyl 6-bromo-2-fluoro-3-methoxy-benzoate

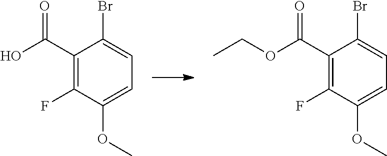

6-Bromo-2-fluoro-3-methoxy-benzoic acid (30.5 g, 123 mmol) was dissolved in MeCN (500 mL). Caesium carbonate (47.9 g, 147 mmol) was added followed by dropwise addition of iodoethane (15.2 mL, 189 mmol). The mixture was stirred at rt for 3 days. The mixture was filtered through Celite, washed with MeCN and concentrated in vacuo. The residue was separated between Et₂O (500 mL) and a brine-water mixture (1:2 brine:water, 750 mL). The aqueous phase was extracted with Et₂O (250 mL). The combined organics were dried over Na₂SO₄ and concentrated in vacuo to afford the title compound as an orange oil that solidified on standing (26.8 g, 79% yield).

### Ethyl 6-((tert-butoxycarbonyl)amino)-2-fluoro-3-methoxybenzoate

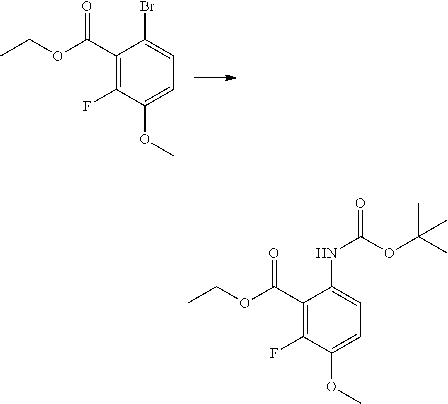 [SMILES: CCOC(=O)c1c(Br)ccc(OC)c1F ; CCOC(=O)c1c(NC(=O)OC(C)(C)C)ccc(OC)c1F]

Ethyl 6-bromo-2-fluoro-3-methoxy-benzoate (10 g, 36 mmol) was dissolved in dioxane (250 mL). tert-Butyl carbamate (4.65 g, 39.7 mmol), 4,5-(bis(diphenylphospheno)-9,9-dimethylxanthene (2.09 g, 3.6 mmol), palladium (II) acetate (810 mg, 3.61 mmol) and caesium carbonate (23.5 g, 72.1 mmol) were added and the mixture was stirred for 18 hrs at 100° C. The mixture was cooled, diluted with EtOAc (250 mL) and filtered through Celite washing with EtOAc (150 mL). The combined filtrates were concentrated in vacuo. Flash chromatography (10% EtOAc, 90% Pet. Ether) afforded the title compound as a colourless oil that solidified on standing (8.45 g, 75% yield).

### Ethyl 6-amino-2-fluoro-3-methoxybenzoate

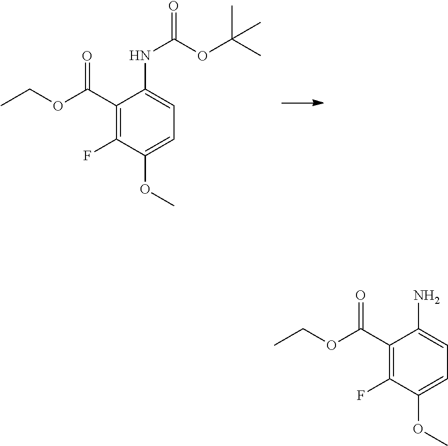 [SMILES: CCOC(=O)c1c(NC(=O)OC(C)(C)C)ccc(OC)c1F ; CCOC(=O)c1c(N)ccc(OC)c1F]

To ethyl 6-((tert-butoxycarbonyl)amino)-2-fluoro-3-methoxybenzoate (3.99 g, 12.7 mmol) was added 4M HCl in dioxane (50 mL) and the mixture stirred at rt for 6 hrs. The mixture was concentrated in vacuo to afford the HCl salt of the title compound as a beige solid. (2.83 g, 89% yield).

### (6-Azido-2-fluoro-3-methoxyphenyl)methanol

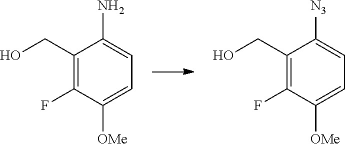 [SMILES: COc1ccc(N)c(CO)c1F ; COc1ccc(N=[N+]=[N-])c(CO)c1F]

A solution of (6-amino-2-fluoro-3-methoxyphenyl)methanol hydrochloride (2.40 g, 11.60 mmol) in methanol (40 mL) was cooled to 0° C. iso-Pentylnitrite (1.60 mL, 11.60 mmol) was added in one portion to the solution, followed by the addition of trimethylsilyl azide (1.60 mL, 11.60 mmol), added slowly over a period of 5 min. After the addition, the mixture was allowed to warm to rt and it was stirred for 3 hrs. The reaction mixture was added to water (100 mL) and methanol was removed in vacuo at 30° C. The mixture was extracted with ethyl acetate (2×100 mL, 1×50 mL), dried over sodium sulfate, filtered and concentrated under reduced pressure at 30° C. The isolated crude material was triturated in the minimum volume of heptane (20 mL). The solid was isolated upon filtration, was washed with heptane and dried to afford the title product (1.85 g, 81% yield).

### Ethyl 1-(3-fluoro-2-(hydroxymethyl)-4-methoxyphenyl)-1H-1,2,3-triazole-4-carboxylate

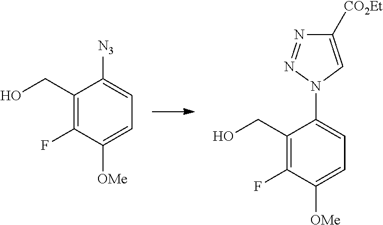 [SMILES: COc1ccc(N=[N+]=[N-])c(CO)c1F ; CCOC(=O)c1cn(-c2ccc(OC)c(F)c2CO)nn1]

Copper (1) iodide (87 mg, 0.457 mmol) and tris[(1-benzyl-1H-1,2,3-triazol-4-yl)methyl]amine (243 mg, 0.457 mmol) were added to a solution of ethyl propiolate (0.55 mL, 5.48 mmol) and (6-azido-2-fluoro-3-methoxyphenyl)methanol (900 mg, 4.57 mmol) in anhydrous acetonitrile (25 mL). The reaction mixture was stirred under nitrogen overnight in darkness. The reaction mixture was concentrated under reduced pressure and then diluted with ethyl acetate (30 mL). The mixture was filtered through a pad of celite and was washed with ethyl acetate (3×30 mL). The filtrates were washed with conc. ammonium chloride solution (30 mL), water (30 mL) and brine (30 mL). The organic layer was dried over sodium sulfate, filtered and concentrated under reduced pressure to give a pale brown solid. The crude material was purified via flash chromatography (50% EtOAc in hexane) to afford the title compound (1.10 g, 82% yield).

### Ethyl 1-(2-(chloromethyl)-3-fluoro-4-methoxyphenyl)-1H-1,2,3-triazole-4-carboxylate

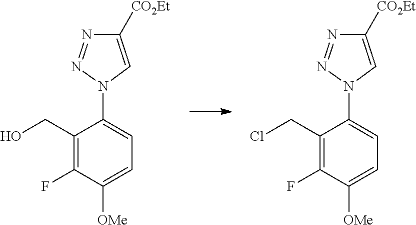 [SMILES: CCOC(=O)c1cn(-c2ccc(OC)c(F)c2CO)nn1 ; CCOC(=O)c1cn(-c2ccc(OC)c(F)c2CCl)nn1]

Triethylamine (0.96 mL, 6.90 mmol) was added to a stirred solution of ethyl 1-(3-fluoro-2-(hydroxymethyl)-4-methoxyphenyl)-1H-1,2,3-triazole-4-carboxylate (1.10 g, 3.73 mmol) in anhydrous dichloromethane (100 mL). The reaction mixture was stirred under nitrogen for 30 min. before adding dropwise methane sulfonyl chloride (0.495 mL, 6.40 mmol) to it. The reaction mixture was stirred at room temperature under nitrogen for 3 hrs. The mixture was partitioned between water (20 mL) and dichloromethane (25 mL). The organic layer was washed with more water (2×20 mL), an aqueous bicarbonate solution (20 mL) and brine (20 mL), then it was dried over sodium sulfate, filtered and concentrated under reduced pressure in order to afford the title compound (1.16 g, 83%).

### Ethyl 1-(2-(((bis-tert-butoxycarbonyl)amino)methyl)-3-fluoro-4-methoxyphenyl)-1H-1,2,3-triazole-4-carboxylate

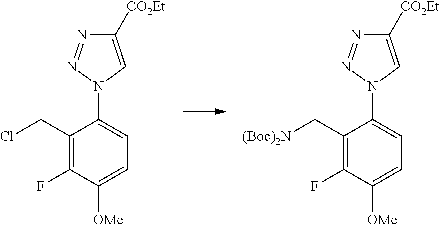

Caesium carbonate (3.04 g, 9.33 mmol) and di-tert-butylaminodicarboxylate (0.679 g, 3.11 mmol) were added to a mixture of ethyl 1-(2-(chloromethyl)-3-fluoro-4-methoxyphenyl)-1H-1,2,3-triazole-4-carboxylate (1.16 g, 3.11 mmol) in dimethylformamide (25 mL). The reaction mixture was stirred at rt for 2 hrs. The mixture was filtered and the filtrates were diluted with water. The aqueous layer was extracted with ethyl acetate (3×25 mL). The organic layers were combined, washed with water (20 mL) and brine (20 mL), dried over sodium sulfate, filtered and concentrated under reduced pressure in order to give the title compound as an orange oil (1.47 g, 96% yield).

### Ethyl 1-(2-(aminomethyl)-3-fluoro-4-methoxyphenyl)-1H-1,2,3-triazole-4-carboxylate Hydrochloride

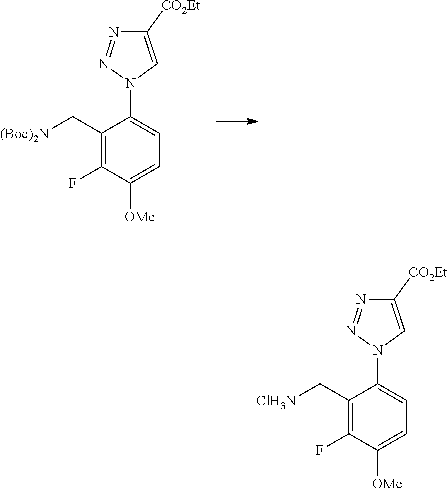

A 4M solution of hydrochloric acid in 1,4-dioxane (15 mL) was added dropwise to a solution of ethyl 1-(2-(((bis-tert-butoxycarbonyl)amino)methyl)-3-fluoro-4-methoxyphenyl)-1H-1,2,3-triazole-4-carboxylate (1.47 g, 2.98 mmol) in 1,4-dioxane (20 mL). The reaction mixture was stirred at rt for 12 hrs. After that time, it was heated to 40° C. for a further 12 hrs. A beige precipitate was isolated upon filtration and was washed with diethyl ether (2×50 mL) and dried in vacuo in order to give the title compound (875 mg, 89% yield).

[M+H]⁺=295.2

### Synthesis of [2-fluoro-3-methoxy-6-[3-(trifluoromethyl)pyrazol-1-yl]phenyl]methanamine

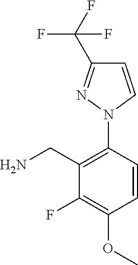 [SMILES: COc1ccc(-n2ccc(C(F)(F)F)n2)c(CN)c1F]

### (2-Fluoro-6-iodo-3-methoxyphenyl)methanol

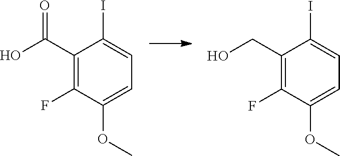

To a solution of 2-fluoro-6-iodo-3-methoxy-benzoic acid (10.0 g, 33.6 mmol) in THF was added 4-methyl morpholine (3.9 mL, 36 mmol) and isobutyl chloroformate (4.4 mL, 34 mmol) dropwise. After 60 min the reaction was filtered and washed with a minimum amount of THF. The filtrate was cooled in an ice-bath and a solution of sodium borohydride (2.0 g, 59 mmol) in cold water (3 mL) was added portion-wise over 20 min. The resulting solution was stirred at rt for 18 hrs. The reaction was acidified with 1M HCl and extracted with TBME. The organic layer was washed sequentially with 2M NaOH(aq), 1M HCl(aq) and brine and dried over MgSO₄ and concentrated in vacuo. Flash chromatography (0-40% EtOAc in Hexanes) afforded the title compound (4.9 g, 49% yield).

### [2-fluoro-3-methoxy-6-[3-(trifluoromethyl)pyrazol-1-yl]phenyl]methanol

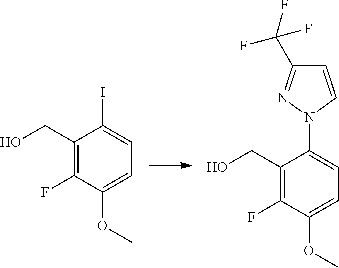 [SMILES: COc1ccc(I)c(CO)c1F ; COc1ccc(-n2ccc(C(F)(F)F)n2)c(CO)c1F]

A mixture of (2-fluoro-6-iodo-3-methoxy-phenyl)methanol (2.0 g, 7.1 mmol), 3-(trifluoromethyl)-1H-pyrazole (1.93 g, 14.2 mmol), (1S,2S)—N1,N2-dimethylcyclohexane-1,2-diamine (1.51 g, 10.6 mmol) and copper(I) iodide (96 mg, 0.50 mmol) was dissolved in DMF (12 mL) then treated with caesium carbonate (3.47 g, 10.7 mmol) and degassed with N₂, then heated at 120° C. for 60 min. The mixture was diluted with DCM (50 mL) and concentrated. Flash chromatography (0 to 75% EtOAc/iso-hexanes) to afforded the title compound (1.02 g, 49% yield).

[M+H]⁺=290.9

1H NMR (DMSO) δ: 3.91 (3H, s), 4.35 (2H, dd, J=5.4, 2.2 Hz), 5.26 (1H, t, J=5.4 Hz), 6.98 (1H, d, J=2.4 Hz), 7.24-7.36 (2H, m), 8.30 (1H, d, J=2.5 Hz)

### 1-[2-(chloromethyl)-3-fluoro-4-methoxyphenyl]-3-(trifluoromethyl)pyrazole

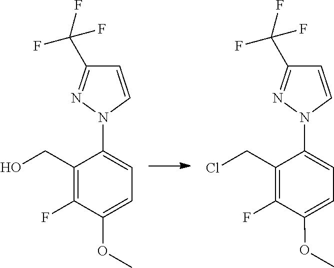 [SMILES: COc1ccc(-n2ccc(C(F)(F)F)n2)c(CO)c1F ; COc1ccc(-n2ccc(C(F)(F)F)n2)c(CCl)c1F]

A stirred solution [2-fluoro-3-methoxy-6-[3-(trifluoromethyl)pyrazol-1-yl]phenyl]methanol (1.02 g, 3.51 mmol) in DCM (25 mL) was treated with triethylamine (0.79 mL, 5.62 mmol) and cooled in an ice-bath under N₂. Methanesulfonyl chloride (0.38 mL, 4.91 mmol) was added slowly then the ice-bath removed and the mixture allowed to warm to rt and stirred for 2 days. The mixture was diluted with DCM (20 mL) and partitioned over saturated NaHCO₃(aq). The aqueous layer was extracted with further DCM. The combined organics were washed with brine (30 mL), dried (Na₂SO₄) and concentrated in vacuo to afford the title compound as a yellow solid (1.13 g, 100% yield).

[M+H]⁺=308.0/310.8

NMR (DMSO) δ: 3.94 (3H, s), 4.66 (2H, d, J=1.8 Hz), 7.04 (1H, d, J=2.5 Hz), 7.34-7.45 (2H, m), 8.26-8.34 (1H, m)

### 2-[[2-fluoro-3-methoxy-6-[3-(trifluoromethyl)pyrazol-1-yl]phenyl]methyl]isoindoline-1,3-dione

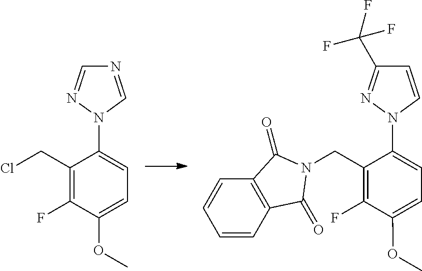 [SMILES: COc1ccc(-n2cncn2)c(CCl)c1F ; COc1ccc(-n2ccc(C(F)(F)F)n2)c(CN2C(=O)c3ccccc3C2=O)c1F]

Potassium phthalimide (0.745 g, 4.02 mmol) was added to a solution of 1-[2-(chloromethyl)-3-fluoro-4-methoxy-phenyl]-3-(trifluoromethyl)pyrazole (1.13 g, 3.66 mmol) in DMF (10 mL) and the mixture warmed to 55° C. for 2 hrs. Water (30 mL) was added to form a thick precipitate which was filtered, washed with water and dried in vacuo in the presence of CaCl₂ to afford the title compound (1.06 g, 68% yield) as a white solid.

[M+H1]=419.8

NMR (DMSO) δ: 3.91 (3H, s), 4.76 (2H, s), 6.86 (1H, d, J=2.5 Hz), 7.20-7.32 (2H, m), 7.71-7.83 (4H, m), 8.20-8.28 (1H, m)

### [2-fluoro-3-methoxy-6-[3-(trifluoromethyl)pyrazol-1-yl]phenyl]methanamine

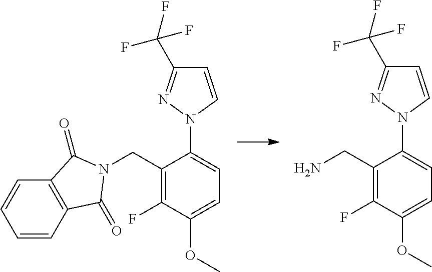 [SMILES: COc1ccc(-n2ccc(C(F)(F)F)n2)c(CN2C(=O)c3ccccc3C2=O)c1F ; COc1ccc(-n2ccc(C(F)(F)F)n2)c(CN)c1F]

Hydrazine hydrate (50-60% solution, 0.45 mL) was added to a suspension of 2-[[2-fluoro-3-methoxy-6-[3-(trifluoromethyl)pyrazol-1-yl]phenyl]methyl]isoindoline-1,3-dione (1.06 g, 2.53 mmol) in MeOH (15 mL) and the reaction mixture heated to 70° C. for 3 hrs. The mixture was filtered and the filtrate concentrated in vacuo. The residue was taken up in TBME (40 mL) and sonicated. This was filtered and the filtrate concentrated in vacuo then dried in vacuo overnight to afford the title compound (554 mg, 75% yield) as a white solid.

[M+H]⁺=289.9

NMR (DMSO) δ: 1.73 (2H, s), 3.46 (2H, d, J=2.2 Hz), 3.91 (3H, s), 7.00 (1H, d, J=2.5 Hz), 7.22 (1H, app t, J=8.9 Hz), 7.31 (1H, dd, J=8.9, 1.6 Hz), 8.34-8.41 (1H, m)

### Synthesis of 6-(Aminomethyl)-7-methoxyisoquinolin-1-amine dihydrochloride

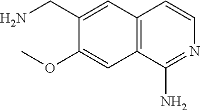

### Tert-Butyl Pivaloyloxycarbamate

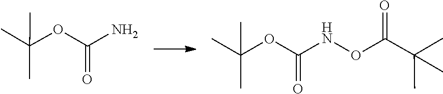

tert-Butyl hydroxycarbamate (15.0 g, 113 mmol) was dissolved in acetonitrile (300 mL, 5746 mmol). Pivalic anhydride (25.4 mL, 124 mmol) was added as a steady stream and the resulting mixture heated to reflux for 18 hrs. The reaction mixture was cooled to rt, then concentrated under vacuum. The residue was partitioned between EtOAc (350 mL) and bicarb (200 mL). The layers were separated and the organic layer washed with Na₂CO₃ (3×100 mL), dried (MgSO₄), filtered and concentrated under reduced pressure to afford the crude product as a white solid, (26.38 g, 86% yield).

Used without characterisation.

### O-Pivaloylhydroxylamine Trifluoromethanesulfonate

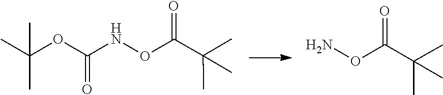

tert-Butyl pivaloyloxycarbamate (26.38 g, 97 mmol) was dissolved in dry diethyl ether (237 mL, 2277 mmol). The reaction mixture was cooled to 0° C. and then triflic acid (8.80 mL, 99 mmol) was added (via syringe) in one portion. The mixture was stirred at 0° C. for 5 min and then allowed to warm to rt. After 60 min iso-hexanes (250 mL) was added and the mixture stirred for 10 min. The resulting solid was filtered, washed with hexanes (3×50 mL) and then dried in the vacuum oven to afford the title compound (24.83 g, 91% yield) as a white powder. Used without characterisation.

### 4-Bromo-3-methoxybenzoyl Chloride

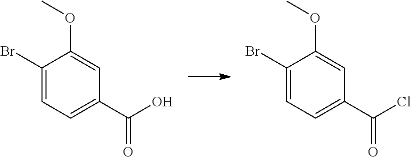

4-Bromo-3-methoxybenzoic acid (0.50 g, 2.164 mmol) was suspended in dry DCM (5.01 mL, 78 mmol). Oxalyl chloride (0.23 mL, 2.60 mmol) was added drop-wise over 5 min. Dry DMF (1 drop) was added. The resulting mixture was stirred at rt 1.5 hrs then solvents were removed under vacuum. The title compound (539 mg, 100% yield) was used directly in the next step.

### 4-Bromo-3-methoxy-N-(pivaloyloxy)benzamide

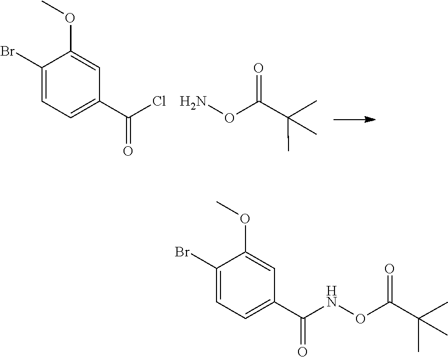

O-Pivaloylhydroxylamine trifluoromethanesulfonate (547 mg, 1.944 mmol) was dissolved in EtOAc (5.5 mL). Water (5.5 mL, 307 mmol) and then sodium carbonate (458 mg, 4.32 mmol) were added. The resulting mixture was cooled to 0° C. and then a solution of 4-bromo-3-methoxybenzoyl chloride (539 mg, 2.16 mmol) in EtOAc (5.5 mL) was added in one portion. The reaction was stirred at 0° C. for 1.5 hrs. The reaction mixture was diluted with EtOAc (10 mL) and quenched with water (5 mL) and Na₂CO₃ (15 mL). The layers were separated and the aqueous was extracted with EtOAc (2×20 mL). The combined organics were washed with brine (20 mL), dried (MgSO₄), filtered and concentrated under reduced pressure. The product was triturated with iso-hexanes (5 mL) and the solvent removed under vacuum to afford the title compound (378 mg, 48% yield) as a white foam.

[M+H]⁺=330.1/332.1

### 6-Bromo-7-methoxyisoquinolin-1 (2H)-one

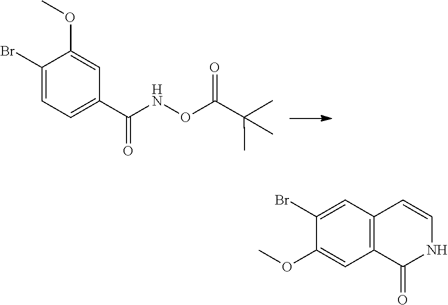

Vinyl acetate (62.8 μL, 0.68 mmol) was added to a N₂ degassed solution of [CpRhCl₂]₂ (5.62 mg, 9.09 μmol), caesium acetate (52.3 mg, 0.27 mmol) and 4-bromo-3-methoxy-N-(pivaloyloxy)benzamide (150 mg, 0.45 mmol) in anhydrous MeOH (1.5 mL) in a sealed microwave vial under a N₂ atmosphere. The reaction was stirred at 50° C. for 90 min. After cooling, the reaction mixture was filtered, washing with a small quantity of MeOH to afford the title compound as a white powder (73 mg, 62% yield).

[M+H]⁺=254.0/256.0

### 6-Bromo-1-chloro-7-methoxyisoquinoline

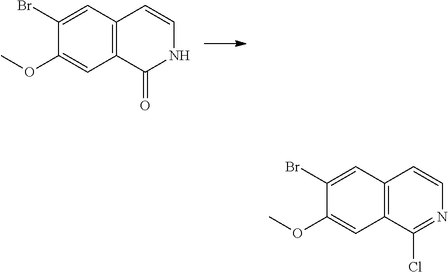

6-Bromo-7-methoxyisoquinolin-1 (2H)-one (2.06 g, 8.11 mmol) was suspended in phosphorus oxychloride (7.56 mL, 81 mmol) in a sealed 24 mL microwave vial and the mixture heated at 100° C., forming a dark solution. After 2 hrs the mixture was quenched carefully into lukewarm water (200 mL). The aqueous was taken to pH 7/8 with sat. aq. NaHCO₃ solution (250 mL). The aqueous was extracted with EtOAc (3×150 mL) and the combined organics washed with brine (100 mL), dried (MgSO₄), filtered and concentrated to afford (2.53 g, 91% yield) as an off-white solid after drying under vacuum.

[M+H]⁺=272.0/274.0

### 6-Bromo-7-methoxyisoquinolin-1-amine

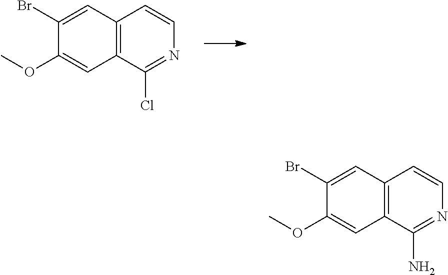

6-Bromo-1-chloro-7-methoxyisoquinoline (500 mg, 1.835 mmol), ammonium acetate (2121 mg, 27.5 mmol) and phenol (2590 mg, 27.5 mmol) were combined in a microwave vial. The mixture was heated thermally to 140° C. for 18 hrs. The mixture was cooled to rt then partitioned between DCM (50 mL) and 2N NaOH (40 mL). The organic layer was collected and the aqueous extracted with further DCM (2×50 mL). The combined organics were dried (Na₂CO₃), filtered and concentrated. The crude product was purified by flash chromatography (0 to 8% MeOH/DCM) to afford the title compound (348 mg, 70% yield) as a brown powder.

[M+H]⁺=253.1/255.0

### 1-Amino-7-methoxyisoquinoline-6-carbonitrile

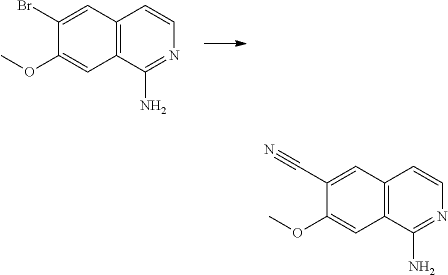

To a degassed solution of dicyanozinc (0.720 g, 6.13 mmol) and 6-bromo-7-methoxyisoquinolin-1-amine (1.35 g, 5.33 mmol) in DMA (24 mL) was added tetrakis(triphenylphosphine)palladium(0) (0.616 g, 0.533 mmol) and the mixture heated to 100° C. for 18 hrs. The mixture was cooled to rt and was poured into water (200 mL), forming a precipitate. This was filtered, washing with water (30 mL). The precipitate was dissolved in 1:1 MeOH/DCM and purified by flash chromatography (SCX, 1% NH₃/MeOH) to afford the title compound (836 mg, 77% yield) as a yellow powder.

[M+H]⁺=200.1 (M+H)+

### tert-Butyl ((1-amino-7-methoxyisoquinolin-6-yl)methyl)carbamate

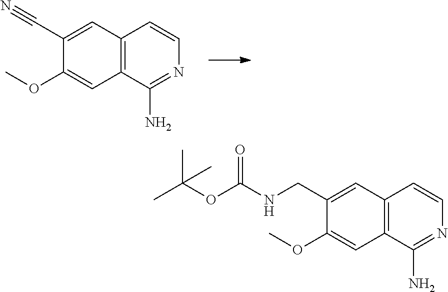

1-Amino-7-methoxyisoquinoline-6-carbonitrile (0.863 g, 4.33 mmol) was dissolved in a mixture of dry methanol (42.9 mL, 1061 mmol) and dry tetrahydrofuran (12.86 ml, 159 mmol) to which nickel chloride hexahydrate (0.105 g, 0.433 mmol) was added followed by di-tert-butyl dicarbonate (1.91 g, 8.66 mmol). The solution was cooled in an ice-salt bath to −5° C. and then sodium borohydride (1.147 g, 30.3 mmol) was added slowly portionwise maintaining the reaction temperature below 0° C. The mixture was stirred at 0° C. for 30 mins, then allowed to warm to rt and stirred for 3 hrs. The solvent was removed under reduced pressure and the resulting solid was taken up in chloroform (100 mL) and washed with sat. aq. NaHCO₃ (100 mL). The aqueous layer was extracted with further chloroform (2×75 mL) and the combined organics washed with water (75 mL) and brine (75 mL), dried (Na₂SO₄), filtered and concentrated. The crude material was purified by flash chromatography (0 to 10% (0.3% NH₃ in MeOH) in DCM) to afford the title compound (512 mg, 37% yield) as a beige powder.

[M+H]⁺=304.2

### 6-(Aminomethyl)-7-methoxyisoquinolin-1-amine dihydrochloride

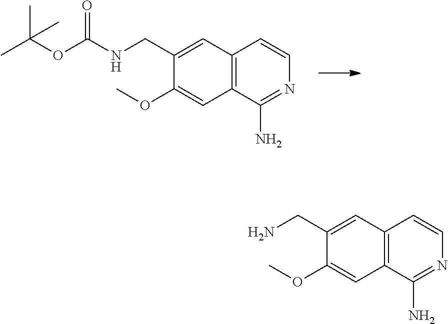 [SMILES: COc1cc2c(N)nccc2cc1CNC(=O)OC(C)(C)C ; COc1cc2c(N)nccc2cc1CN]

tert-Butyl ((1-amino-7-methoxyisoquinolin-6-yl)methyl)carbamate (0.508 g, 1.675 mmol) was reacted following general method A at 40° C. for 2 hrs to afford the title compound (449 mg, 94% yield) as a beige powder.

[M+H]⁺=204.2

### 6-(Aminomethyl)isoquinolin-1-amine CAS 215454-95-8

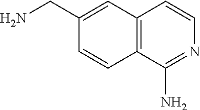 [SMILES: NCc1ccc2c(N)nccc2c1]

The title compound was synthesised according to procedures detailed in WO2016083816

### Synthesis of tert-butyl (6-(aminomethyl)isoquinolin-1-yl)(tert-butoxycarbonyl)carbamate

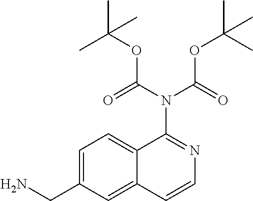 [SMILES: CC(C)(C)OC(=O)N(C(=O)OC(C)(C)C)c1nccc2cc(CN)ccc12]

### 2-Trimethylsilylethyl N-[(1-amino-6-isoquinolyl)methyl]carbamate

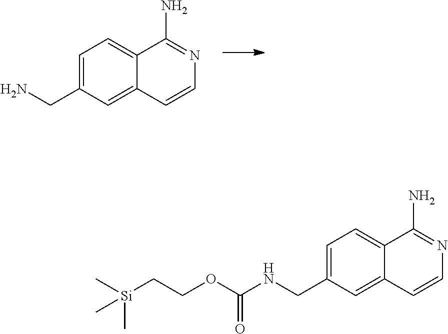 [SMILES: NCc1ccc2c(N)nccc2c1 ; C[Si](C)(C)CCOC(=O)NCc1ccc2c(N)nccc2c1]

6-(Aminomethyl)isoquinolin-1-amine dihydrochloride (85 g, 345 mmol) was stirred in a mixture of water (0.446 L) and DMF (1.36 L). The reaction vessel was cooled in an ice-bath before the addition of triethylamine (87.4 g, 863 mmol) and (2,5-dioxopyrrolidin-1-yl) 2-trimethylsilylethyl carbonate (98.5 g, 380 mmol). The mixture was stirred at rt for 18 hrs. Solvents were removed under vacuum. The mixture was partitioned between EtOAc (450 mL), water (75 mL) and 2N NaOH (500 mL). The aqueous was extracted with further EtOAc (4×125 mL) and the combined organics washed with brine (100 mL), dried (Na₂SO₄), filtered and concentrated. The residue was dried under vacuum then triturated with 2:1 Et₂O/Iso-Hexanes (375 mL) to afford the title compound (93.2 g, 82% yield) as a pale yellow powder.

[M+H]⁺=318.4

### tert-Butyl N-tert-butoxycarbonyl-N-[6-[(2-trimethylsilylethoxycarbonylamino)methyl]-1-isoquinolyl]carbamate

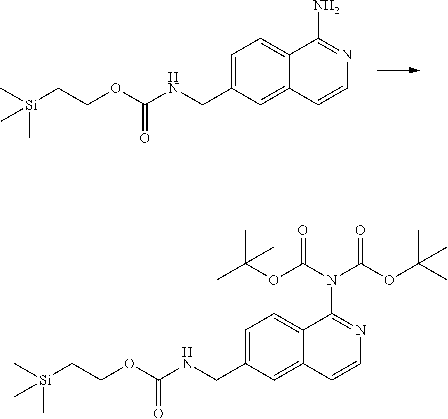 [SMILES: C[Si](C)(C)CCOC(=O)NCc1ccc2c(N)nccc2c1 ; CC(C)(C)OC(=O)N(C(=O)OC(C)(C)C)c1nccc2cc(CNC(=O)OCC[Si](C)(C)C)ccc12]

A mixture of di-tert-butyl dicarbonate (215 g, 986 mmol) and 2-trimethylsilylethyl N-[(1-amino-6-isoquinolyl)methyl]carbamate (31.3 g, 98.6 mmol) in anhydrous tert-butanol (283 mL) was heated at 66° C. for 48 hrs. Solvents were removed under vacuum. The crude material was purified by flash chromatography (0-50% EtOAc/Iso-Hexanes) to afford the title compound (33.9 g, 60% yield) as a sticky yellow gum.

[M+H]⁺=518.3

### tert-Butyl N-[6-(aminomethyl)-1-isoquinolyl]-N-tert-butoxycarbonyl-carbamate

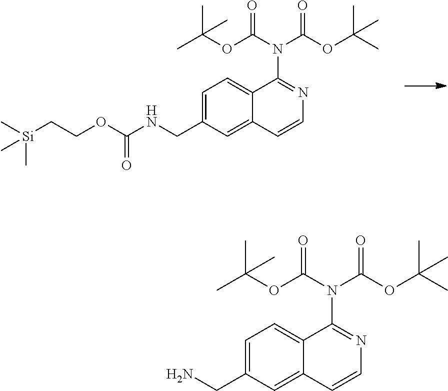 [SMILES: CC(C)(C)OC(=O)N(C(=O)OC(C)(C)C)c1nccc2cc(CNC(=O)OCC[Si](C)(C)C)ccc12 ; CC(C)(C)OC(=O)N(C(=O)OC(C)(C)C)c1nccc2cc(CN)ccc12]

A solution of tert-butyl N-tert-butoxycarbonyl-N-[6-[(2-trimethylsilylethoxycarbonylamino)methyl]-1-isoquinolyl]carbamate (31.9 g, 55.5 mmol) in THF (358 mL) was treated with tetra-nbutylammonium fluoride (185 mL, 185 mmol) at a steady stream via dropping funnel, and the mixture stirred at rt for 6 hrs. The residue was partitioned between EtOAc (1 L) and water (500 mL) containing brine (100 mL). The organic layer was washed with water (150 mL) containing brine (50 mL). The aqueous was then extracted with EtOAc (8×250 mL). The combined organics were dried (Na₂SO₄), filtered and concentrated. The residue was purified by flash chromatography (0 to 6% MeOH (1% NH₃) in DCM). The isolated solids were triturated with water (75 mL) for 3 hrs until a fine solid, then filtered and dried under vacuum in the presence of CaCl₂ to afford the title compound (12.9 g, 59% yield) as a yellow solid.

[M+H]⁺=374.2

### (3-Chloro-1H-pyrrolo[2,3-b]pyridin-5-yl)methanamine (CAS 754173-67-6)

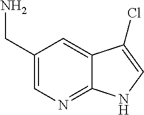 [SMILES: NCc1cnc2[nH]cc(Cl)c2c1]

The title compound was synthesised according to procedures detailed in WO2016083816

### (3-Chloro-1H-indol-5-yl)methanamine (CAS 267875-64-9)

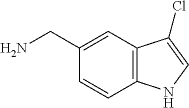 [SMILES: NCc1ccc2[nH]cc(Cl)c2c1]

The title compound was synthesised according to procedures detailed in WO2000026211

### 6-(Aminomethyl)-5-methylisoquinolin-1-amine

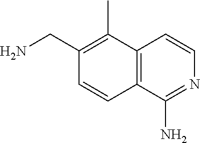

The title compound was synthesised according to procedures detailed in WO2016083816

### 4-(Aminomethyl)benzimidamide (CAS 226942-83-2)

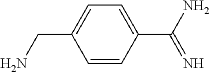 [SMILES: N=C(N)c1ccc(CN)cc1]

The title compound was synthesised and used Boc protected, according to procedures in WO2000069834.

### 5-(Aminomethyl)thiophene-2-carboximidamide (CAS 308846-05-1)

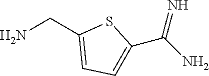

The title compound was synthesised and used Z protected, according to procedures in WO2000069834

The following intermediates are commercial from reliable and well known suppliers:
3-Chloro-4-methoxy-benzylamine: CAS 115514-77-7
(1H-Indol-4-yl)methanamine: CAS 3468-18-6
(1H-Indol-5-yl)methanamine: CAS 81881-74-5
(1H-Pyrrolo[2,3b]pyridin-5-yl)methanamine: CAS 267876-25-5
(1H-Indazol-5-yl)methanamine: CAS 267413-25-2
(1-Methyl-1H-indazol-5-yl)methanamine: CAS 267413-27-4
(5R)-5H,6H,7H-cyclopenta[c]pyridine-1,5-diamine dihydrochloride: CAS 2096419-45-1
4-(aminomethyl)pyridin-2-amine: CAS 199296-51-0
4-(2-aminoethyl)pyridin-2-amine dihydrochloride: CAS 165528-71-2
tert-Butyl N-[4-(aminomethyl)benzyl]carbamate: CAS 108468-00-4
1-(pyridin-4-yl)piperidin-4-amine: CAS 187084-44-2

### Specific Examples of the Present Invention

### Example 0.64

### (S)—N-(1-(((1-Aminoisoquinolin-6-yl)methyl)amino)-3-(3,4-difluorophenyl)-1-oxopropan-2-yl)-7-isopropyl-[1,2,4]triazolo[1,5-a]pyrimidine-5-carboxamide

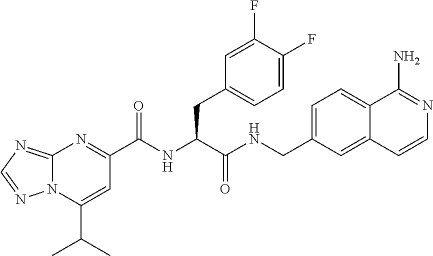 [SMILES: CC(C)c1cc(C(=O)N[C@@H](Cc2ccc(F)c(F)c2)C(=O)NCc2ccc3c(N)nccc3c2)nc2ncnn12]

### tert-Butyl N-tert-butoxycarbonyl-N-[6-[[[(2S)-3-(3,4-difluorophenyl)-2-(2-trimethylsilylethoxycarbonylamino)propanoyl]amino]methyl]-1-isoquinolyl]carbamate

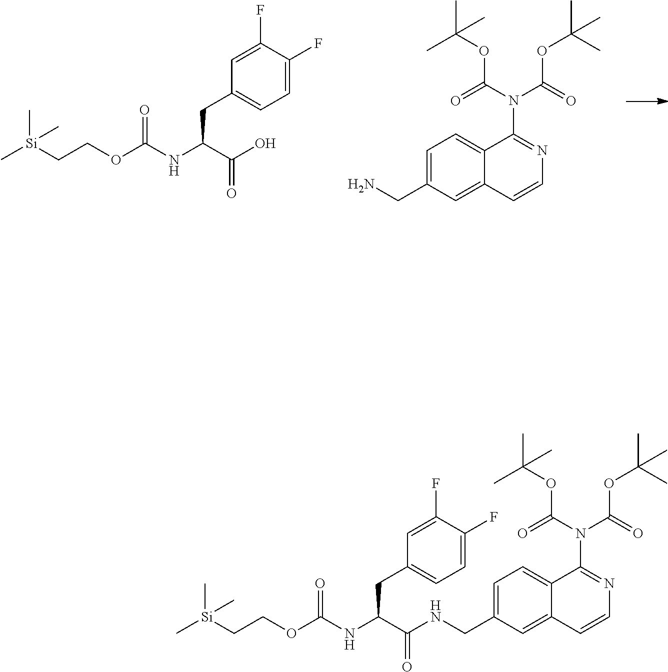 [SMILES: C[Si](C)(C)CCOC(=O)N[C@@H](Cc1ccc(F)c(F)c1)C(=O)O ; CC(C)(C)OC(=O)N(C(=O)OC(C)(C)C)c1nccc2cc(CN)ccc12 ; CC(C)(C)OC(=O)N(C(=O)OC(C)(C)C)c1nccc2cc(CNC(=O)[C@H](Cc3ccc(F)c(F)c3)NC(=O)OCC[Si](C)(C)C)ccc12]

Following general method C (i) (2S)-3-(3,4-difluorophenyl)-2-(2-trimethylsilylethoxycarbonylamino) propanoic acid (20.8 g, 60.1 mmol) was reacted with tert-butyl N-[6-(aminomethyl)-1-isoquinolyl]-N-tert-butoxycarbonyl carbamate (22.5 g, 60.1 mmol). Flash chromatography (0-35% EtOAc/Iso-hexanes) afforded the title compound (36.06 g, 75% yield) as an off-white powder.

[M+H]⁺=701.3

### tert-Butyl N-tertbutoxycarbonyl-N-[6-[[[(2S)-2-amino-3-(3,4-difluorophenyl)propanoyl]amino]methyl]-1-isoquinolyl]carbamate

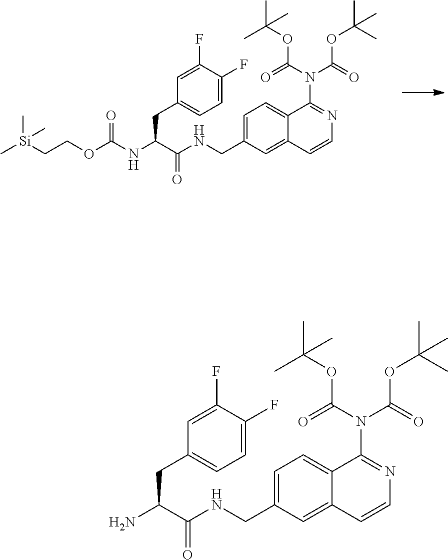 [SMILES: CC(C)(C)OC(=O)N(C(=O)OC(C)(C)C)c1nccc2cc(CNC(=O)[C@H](Cc3ccc(F)c(F)c3)NC(=O)OCC[Si](C)(C)C)ccc12 ; CC(C)(C)OC(=O)N(C(=O)OC(C)(C)C)c1nccc2cc(CNC(=O)[C@@H](N)Cc3ccc(F)c(F)c3)ccc12]

A solution of tert-butyl N-tert-butoxycarbonyl-N-[6-[[[(2S)-3-(3,4-difluorophenyl)-2-(2-trimethylsilylethoxycarbonylamino)propanoyl]amino]methyl]-1isoquinolyl]carbamate (36.1 g, 45.3 mmol) in dry THF (314 mL) was treated dropwise with a 1M solution of TBAF (136 mL, 136 mmol). The mixture was stirred at rt for 18 hrs. Solvents were removed under vacuum and the residue partitioned between EtOAc (250 mL) and a mixture of water (170 mL) and brine (170 mL). The aqueous layer was extracted with further EtOAc (2×250 mL) and the combined organics dried (Na₂SO₄), filtered and concentrated. The residue was purified by flash chromatography (0 to 5% MeOH (1% NH₃) in DCM) to afford the title compound (20.99 g, 79% yield).

### tert-Butyl N-tert-butoxycarbonyl-N-[6-[[[(2R)-3-(3,4-difluorophenyl)-2-[(7-isopropyl-[1,2,4]triazolo[1,5-a]pyrimidine-5-carbonyl)amino]propanoyl]amino]methyl]-1-isoquinolyl]carbamate

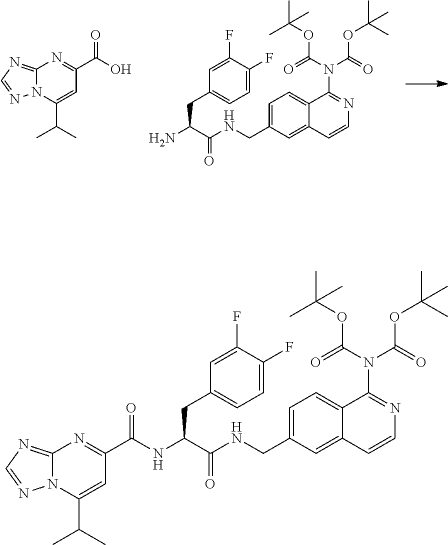 [SMILES: CC(C)c1cc(C(=O)O)nc2ncnn12 ; CC(C)(C)OC(=O)N(C(=O)OC(C)(C)C)c1nccc2cc(CNC(=O)[C@@H](N)Cc3ccc(F)c(F)c3)ccc12 ; CC(C)c1cc(C(=O)N[C@@H](Cc2ccc(F)c(F)c2)C(=O)NCc2ccc3c(N(C(=O)OC(C)(C)C)C(=O)OC(C)(C)C)nccc3c2)nc2ncnn12]

Following general method C(i) 7-isopropyl-[1,2,4]triazolo[1,5-a]pyrimidine-5-carboxylic acid (111 mg, 0.54 mmol) was coupled to tert-butyl N-tert-butoxycarbonyl-N-[6-[[[(2R)-2-amino-3-(3,4-difluorophenyl)propanoyl]amino]methyl]-1-isoquinolyl]carbamate (300 mg, 0.54 mmol). The crude product was purified by flash chromatography (0-100% EtOAc/Iso-Hexanes) to afford the title compound (305 mg, 76% yield).

[M+Na]⁺=767.5

### (S)—N-(1-(((1-aminoisoquinolin-6-yl)methyl)amino)-3-(3,4-difluorophenyl)-1-oxopropan-2-yl)-7-isopropyl-[1,2,4]triazolo[1,5-a]pyrimidine-5-carboxamide

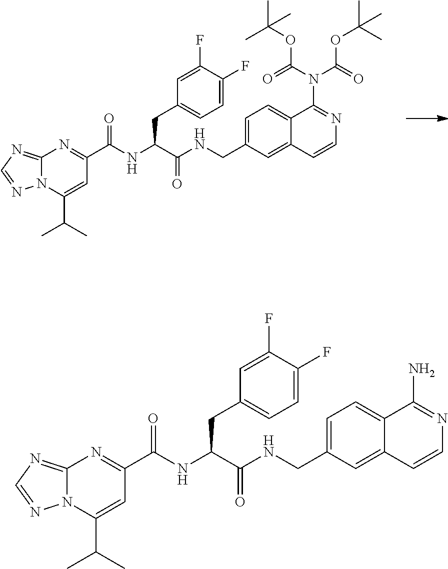 [SMILES: CC(C)c1cc(C(=O)N[C@@H](Cc2ccc(F)c(F)c2)C(=O)NCc2ccc3c(N(C(=O)OC(C)(C)C)C(=O)OC(C)(C)C)nccc3c2)nc2ncnn12 ; CC(C)c1cc(C(=O)N[C@@H](Cc2ccc(F)c(F)c2)C(=O)NCc2ccc3c(N)nccc3c2)nc2ncnn12]

Following a modification of general method A, a solution of tert-butyl N-tert-butoxycarbonyl-N-[6-[[[(2S)-3-(3,4-difluorophenyl)-2-[(7-isopropyl-[1,2,4]triazolo[1,5-a]pyrimidine-5 carbonyl)amino]propanoyl]amino]methyl]-1-isoquinolyl]carbamate (296 mg, 0.397 mmol) in anhydrous DCM (6 mL) was treated with trifluoroacetic acid (2.01 mL, 26 mmol). Flash chromatography (0 to 7% (1% NH₃ in MeOH) in DCM) afforded the title compound (175 mg, 80% yield) as a pale yellow powder.

[M+H]⁺=545.4

(DMSO) 1.40 (6H, d, J=6.9 Hz), 3.22 (2H, d, J=7.2 Hz), 3.76 (1H, sept, J=6.9 Hz), 4.37-4.53 (2H, m), 4.86 (1H, dt, J=8.6, 7.2 Hz), 6.74 (2H, s), 6.82 (1H, d, J=6.0 Hz), 7.09-7.15 (1H, m), 7.27 (1H, dt, J=10.9, 8.5 Hz), 7.32-7.42 (2H, m), 7.51 (1H, s), 7.66 (1H, s), 7.76 (1H, d, J=5.8 Hz), 8.13 (1H, d, J=8.6 Hz), 8.77 (1H, t, J=5.9 Hz), 8.86 (1H, s), 9.23 (1H, d, J=8.7 Hz).

### Example 3.104

### (2S)—N-[(3-amino-1H-indazol-6-yl)methyl]-3-(3,4-difluorophenyl)-2-{2-[(2R,6S)-2,6-dimethylpiperidin-1-yl]acetamido}propanamide

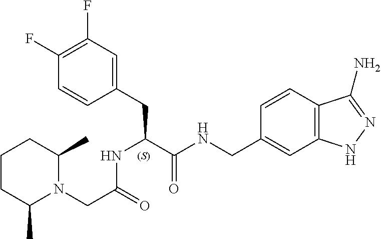

### (2S)—N-[(4-cyano-3-fluorophenyl)methyl]-3-(3,4-difluorophenyl)-2-{2-[(2R,6S)-2,6-dimethylpiperidin-1-yl]acetamido}propanamide

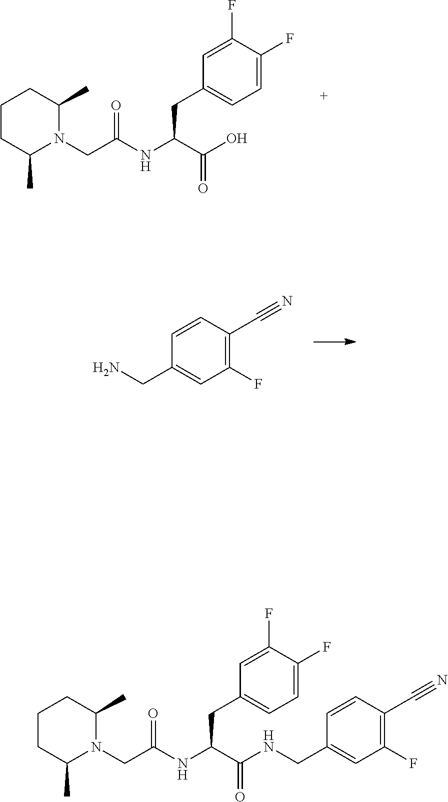 [SMILES: C[C@@H]1CCC[C@H](C)N1CC(=O)N[C@@H](Cc1ccc(F)c(F)c1)C(=O)O ; N#Cc1ccc(CN)cc1F ; C[C@@H]1CCC[C@H](C)N1CC(=O)N[C@@H](Cc1ccc(F)c(F)c1)C(=O)NCc1ccc(C#N)c(F)c1]

(2S)-3-(3,4-Difluorophenyl)-2-{2-[(2R,6S)-2,6-dimethylpiperidin-1-yl]acetamido}propanoic acid (250 mg, 0.71 mmol) was reacted with 4-(Aminomethyl)-2-fluorobenzonitrile (145 mg, 0.78 mmol) following general conditions C(ii) to give the title compound a white solid (320 mg, 93% yield).

[M+H]⁺=487.4

### (2S)—N-[(3-amino-1H-indazol-6-yl)methyl]-3-(3,4-difluorophenyl)-2-{2-[(2R,6S)-2,6-dimethylpiperidin-1-yl]acetamido}propanamide

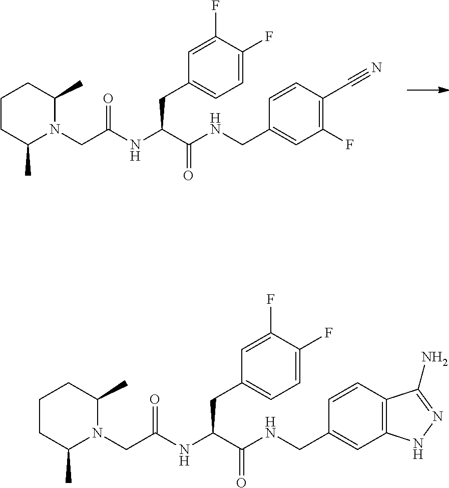 [SMILES: C[C@@H]1CCC[C@H](C)N1CC(=O)N[C@@H](Cc1ccc(F)c(F)c1)C(=O)NCc1ccc(C#N)c(F)c1 ; C[C@@H]1CCC[C@H](C)N1CC(=O)N[C@@H](Cc1ccc(F)c(F)c1)C(=O)NCc1ccc2c(N)n[nH]c2c1]

(2S)—N-[(4-cyano-3-fluorophenyl)methyl]-3-(3,4-difluorophenyl)-2-{2-[(2R,6S)-2,6-dimethylpiperidin-1-yl]acetamido}propanamide (120 mg, 0.25 mmol) was dissolved in n-butanol (25 mL) under nitrogen. Hydrazine hydrate (951 mg, 12.33 mmol) was added and the reaction mixture was stirred at 120° C. for 60 min after which time the solvent was removed in vacuo. The residue was diluted with EtOAc (100 mL), this solution was washed with water (30 mL), brine (30 mL), dried (Na₂SO₄) and filtered through PS paper and evaporated in vacuo. The residue was purified by flash chromatography (0-8% MeOH in CHCl₃). The residue was dissolved in 1M HCl in MeOH (10 mL), evaporated in vacuo and freeze dried from MeCN/water to give a white solid identified as the title compound (62 mg, 47% yield).

[M+H]⁺=499.6

1H NMR (d6-DMSO) δ: 0.82 (3H, d, J=6.5 Hz), 1.10 (3H, t, J=6.5 Hz), 1.28-1.34 (1H, m), 1.43-1.54 (1H, m), 1.60-1.70 (4H, m), 2.80 (1H, dd, J=10.5, 13.7 Hz), 3.11-3.18 (1H, m), 3.34-3.36 (1H, m), 3.79-3.96 (2H, m), 4.42 (2H, d, J=5.8 Hz), 4.69-4.75 (1H, m), 7.04 (1H, d, J=8.5 Hz), 7.13 (1H, s), 7.25-7.38 (4H, m), 7.90 (1H, d, J=8.4 Hz), 8.78 (1H, s), 8.93 (1H, t, J=5.8 Hz), 9.02 (1H, d, J=5.1 Hz), 9.06 (1H, d, J=11.9 Hz), 9.70 (1H, s)

### Example 3.105

### (2S)—N-[(3-amino-1,2-benzoxazol-6-yl)methyl]-3-(3,4-difluorophenyl)-2-{2-[(2R,6S)-2,6-dimethylpiperidin-1-yl]acetamido}propanamide

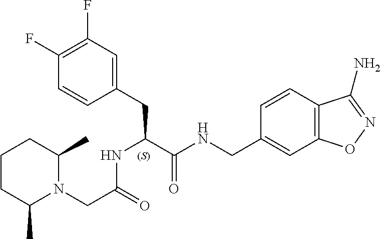

### (2S)—N-[(3-amino-1,2-benzoxazol-6-yl)methyl]-3-(3,4-difluorophenyl)-2-{2-[(2R,6S)-2,6-dimethylpiperidin-1-yl]acetamido}propanamide

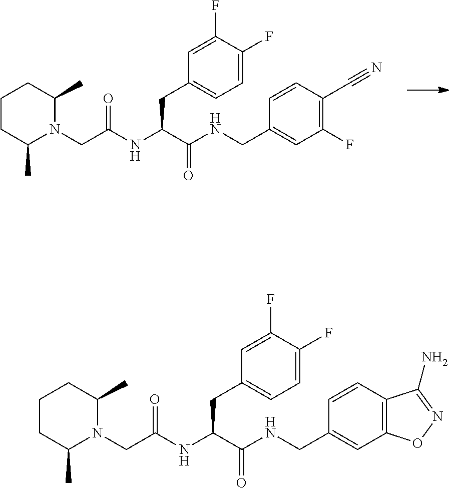 [SMILES: C[C@@H]1CCC[C@H](C)N1CC(=O)N[C@@H](Cc1ccc(F)c(F)c1)C(=O)NCc1ccc(C#N)c(F)c1 ; C[C@@H]1CCC[C@H](C)N1CC(=O)N[C@@H](Cc1ccc(F)c(F)c1)C(=O)NCc1ccc2c(N)noc2c1]

(2S)—N-[(4-cyano-3-fluorophenyl)methyl]-3-(3,4-difluorophenyl)-2-{2-[(2R,6S)-2,6-dimethylpiperidin-1-yl]acetamido}propanamide (120 mg, 0.25 mmol) was dissolved in DMF (15 mL) and water (1.5 mL) under nitrogen. Acetohydroxamic acid (111 mg, 1.48 mmol) and potassium carbonate (409 mg, 2.96 mmol) were added and the reaction mixture was stirred at 55° C. for 18 hrs. The reaction mixture was diluted with EtOAc (100 mL) and washed with water (30 mL), brine (30 mL), dried (Na₂SO₄) and filtered through PS paper and evaporated in vacuo. The residue was purified by flash chromatography (0-8% MeOH in CHCl₃). The residue was dissolved in 1M HCl in MeOH (10 mL), concentrated and freeze dried from MeCN/water to give a white solid identified as the title compound (42 mg, 32% yield).

[M+H]⁺=500.5

1H NMR (d6-DMSO) δ: 0.82 (3H, d, J=6.6 Hz), 1.11 (3H, t, J=6.5 Hz), 1.29-1.35 (1H, m), 1.44-1.51 (1H, m), 1.60-1.71 (4H, m), 2.79-2.84 (1H, m), 3.10-3.16 (1H, m), 3.34-3.35 (1H, m), 3.60-3.94 (2H, m), 4.42 (2H, d, J=5.7 Hz), 4.68-4.76 (1H, m), 7.11 (2H, t, J=0.8 Hz), 7.25 (1H, s), 7.26-7.38 (2H, m), 7.75 (1H, dd, J=2.1, 8.1 Hz), 8.80 (1H, s), 8.85 (1H, t, J=6.0 Hz), 8.94-9.04 (2H, m), 9.32 (1H, s)

### Example 9.07

### (2R)—N-[(1S)-1-[({4-Aminothieno[3,2-c]pyridin-2-yl}methyl)carbamoyl]ethyl]-2-amino-4-phenylbutanamide

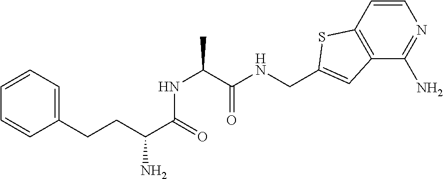 [SMILES: C[C@H](NC(=O)[C@H](N)CCc1ccccc1)C(=O)NCc1cc2c(N)nccc2s1]

### tert-butyl N-[(1S)-1-[({4-aminothieno[3,2-c]pyridin-2-yl}methyl)carbamoyl]ethyl]carbamate

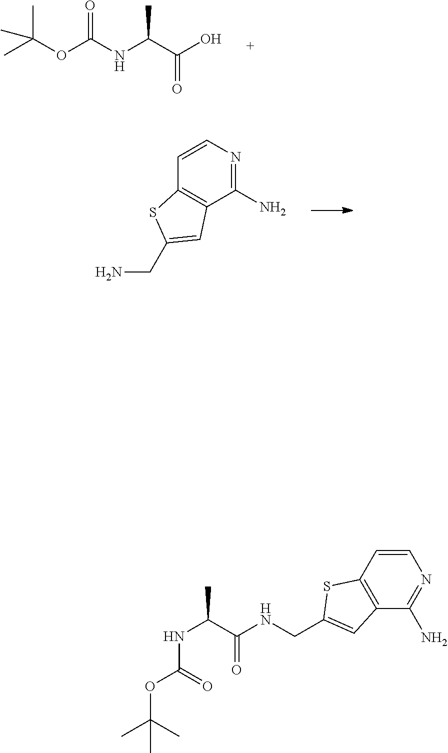 [SMILES: C[C@H](NC(=O)OC(C)(C)C)C(=O)O ; NCc1cc2c(N)nccc2s1 ; C[C@H](NC(=O)OC(C)(C)C)C(=O)NCc1cc2c(N)nccc2s1]

tert-Butyl N-[(1S)-1-[({4-aminothieno[3,2-c]pyridin-2-yl}methyl)carbamoyl]ethyl]carbamate was prepared from Boc-Ala-OH and 2-(aminomethyl)thieno[3,2-c]pyridin-4-amine according to general method C (i) (394 mg, 81% yield).

[M+H]⁺=351.2

### (2S)-2-Amino-N-({4-aminothieno[3,2-c]pyridin-2-yl}methyl)propanamide Dihydrochloride

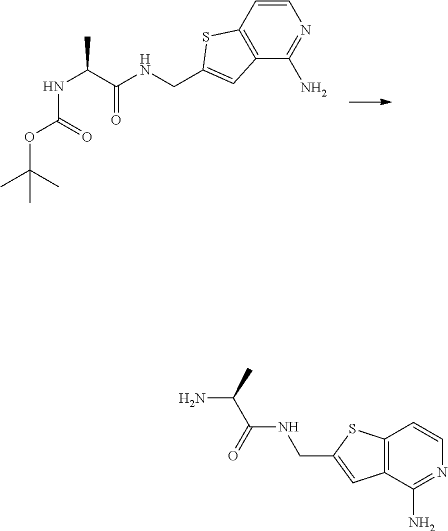 [SMILES: C[C@H](NC(=O)OC(C)(C)C)C(=O)NCc1cc2c(N)nccc2s1 ; C[C@H](N)C(=O)NCc1cc2c(N)nccc2s1]

(2S)-2-Amino-N-({4-aminothieno[3,2-c]pyridin-2-yl}methyl)propanamide dihydrochloride was prepared from tert-butyl N-[(1S)-1-[({4-aminothieno[3,2-c]pyridin-2-yl}methyl)carbamoyl]ethyl]carbamate to general method A (i) (257 mg, 100% yield).

[M+H]⁺=251.1

### tert-Butyl N-[(1R)-1-{[(1S)-1-[({4-aminothieno[3,2-c]pyridin-2-yl}methyl)carbamoyl]ethyl]carbamoyl}-3-phenylpropyl]carbamate

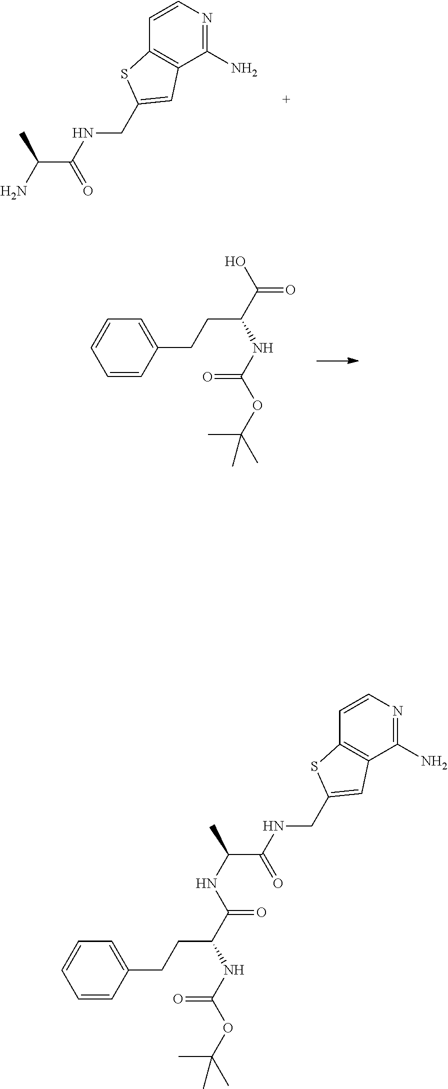 [SMILES: C[C@H](N)C(=O)NCc1cc2c(N)nccc2s1 ; CC(C)(C)OC(=O)N[C@H](CCc1ccccc1)C(=O)O ; C[C@H](NC(=O)[C@@H](CCc1ccccc1)NC(=O)OC(C)(C)C)C(=O)NCc1cc2c(N)nccc2s1]

tert-Butyl N-[(1R)-1-{[(1S)-1-[({4-aminothieno[3,2-c]pyridin-2-yl}methyl)carbamoyl]ethyl]carbamoyl}-3-phenylpropyl]carbamate was prepared from Boc-Hph-OH and (2S)-2-amino-N-({4-aminothieno[3,2-c]pyridin-2-yl}methyl)propanamide dihydrochloride according to general method C (i) (520 mg, 98% yield).

[M+H]⁺=412.4

### (2R)—N-[(1S)-1-[({4-Aminothieno[3,2-c]pyridin-2-yl}methyl)carbamoyl]ethyl]-2-amino-4-phenylbutanamide Dihydrochloride

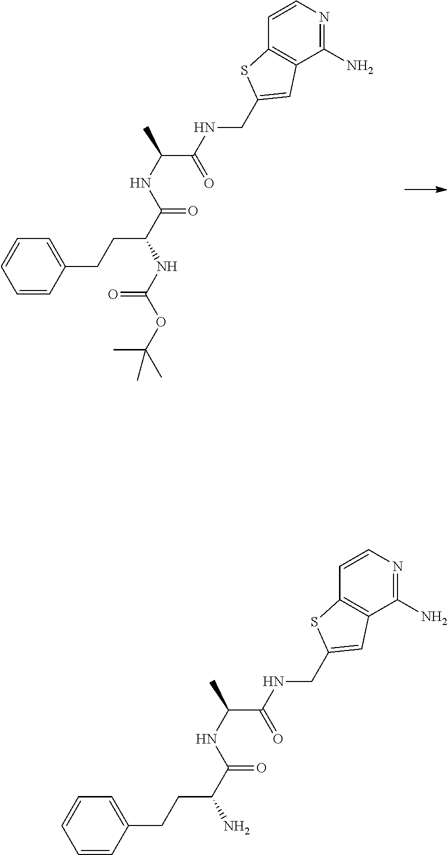 [SMILES: C[C@H](NC(=O)[C@@H](CCc1ccccc1)NC(=O)OC(C)(C)C)C(=O)NCc1cc2c(N)nccc2s1 ; C[C@H](NC(=O)[C@H](N)CCc1ccccc1)C(=O)NCc1cc2c(N)nccc2s1]

(2R)—N-[(1S)-1-[({4-Aminothieno[3,2-c]pyridin-2-yl}methyl)carbamoyl]ethyl]-2-amino-4-phenylbutanamide dihydrochloride was prepared from tert butyl N-[(1R)-1-{[(1S)-1-[({4-aminothieno[3,2-c]pyridin-2-yl}methyl)carbamoyl]ethyl]carbamoyl}-3-phenylpropyl]carbamate A (i) (12 mg, 3% yield).

[M+H]⁺=251.1

¹H NMR (400 MHz, DMSO): 1.28-1.25 (3H, m), 1.69-1.58 (1H, m), 1.94-1.83 (1H, m), 2.09-2.03 (2H, m), 2.79-2.55 (2H, m), 3.22-3.15 (1H, m), 4.33 (1H, t, J=6.1 Hz), 4.53-4.47 (2H, m), 6.45 (2H, s), 7.01 (1H, d, J=6.0 Hz), 7.21-7.17 (3H, m), 7.30-7.25 (2H, m), 7.46 (1H, s), 7.71 (1H, d, J=5.6 Hz), 8.19-8.14 (1H, m), 8.66 (1H, t, J=5.9 Hz).

### Example 9.27

### (2R)—N-[(1S)-1-[({4-Aminothieno[3,2-c]pyridin-2-yl}methyl)carbamoyl]ethyl]-2-(isopropylamino)-4-phenylbutanamide

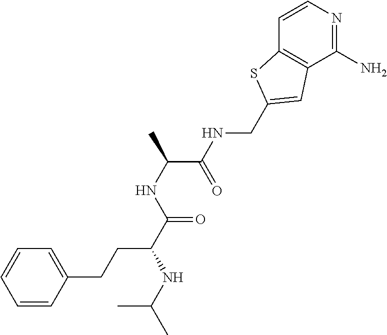 [SMILES: CC(C)N[C@H](CCc1ccccc1)C(=O)N[C@@H](C)C(=O)NCc1cc2c(N)nccc2s1]

(2R)—N-[(1S)-1-[({4-Aminothieno[3,2-c]pyridin-2-yl}methyl)carbamoyl]ethyl]-2-(isopropylamino)-4-phenylbutanamide

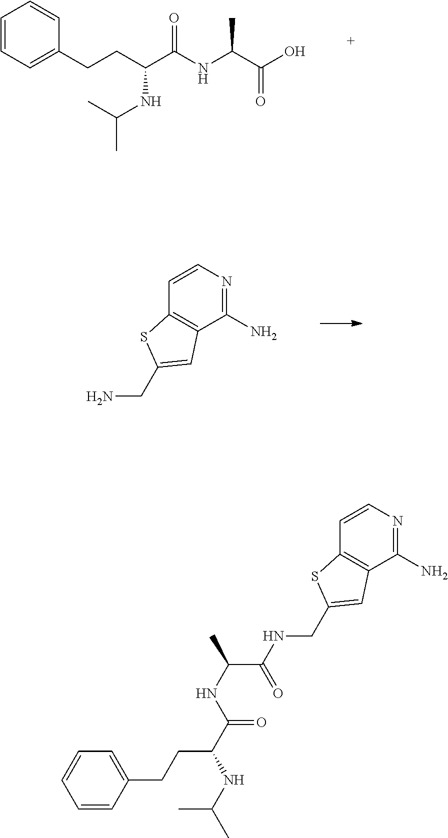 [SMILES: CC(C)N[C@H](CCc1ccccc1)C(=O)N[C@@H](C)C(=O)O ; NCc1cc2c(N)nccc2s1 ; CC(C)N[C@H](CCc1ccccc1)C(=O)N[C@@H](C)C(=O)NCc1cc2c(N)nccc2s1]

(2R)—N-[(1S)-1-[({4-Aminothieno[3,2-c]pyridin-2-yl}methyl)carbamoyl]ethyl]-2-(isopropylamino)-4-phenylbutanamide was prepared from (2S)-2-[(2R)-2-(isopropylamino)-4-phenylbutanamido]propanoic acid and 2-(aminomethyl)thieno[3,2-c]pyridin-4-amine according to general method C (ii) (24 mg, 25% yield).

[M+H]⁺=454.1

NMR 1H (DMSO, 400M Hz): 0.92-0.96 (6H, m), 1.24 (3H, d, J=7.0 Hz), 1.61-1.70 (1H, m), 1.72-1.82 (1H, m), 2.15 (1H, s), 2.54-2.67 (3H, m), 3.09-3.13 (1H, m), 4.31-4.39 (1H, m), 4.44-4.55 (2H, m), 6.46 (2H, s), 6.98 (1H, d, J=5.7 Hz), 7.14-7.18 (3H, m), 7.24-7.28 (2H, m), 7.45 (1H, s), 7.70 (1H, d, J=5.6 Hz), 8.19 (2H, s), 8.21 (1H, s), 8.68 (1H, t, J=5.8 Hz)

### Example 10.01

### (2S)—N-[(3-Chloro-1H-indol-5-yl)methyl]-1-[(2R)-2-(isopropylamino)-6-(piperidin-1-yl)hexanoyl]azetidine-2-carboxamide

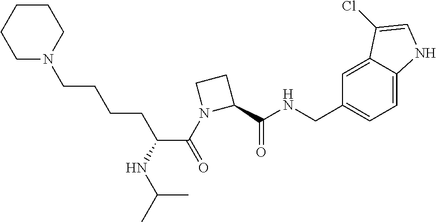 [SMILES: CC(C)N[C@H](CCCCN1CCCCC1)C(=O)N1CC[C@H]1C(=O)NCc1ccc2[nH]cc(Cl)c2c1]

### tert-Butyl (2S)-2-{[(3-Chloro-1H-indol-5-yl)methyl]carbamoyl}azetidine-1-carboxylate

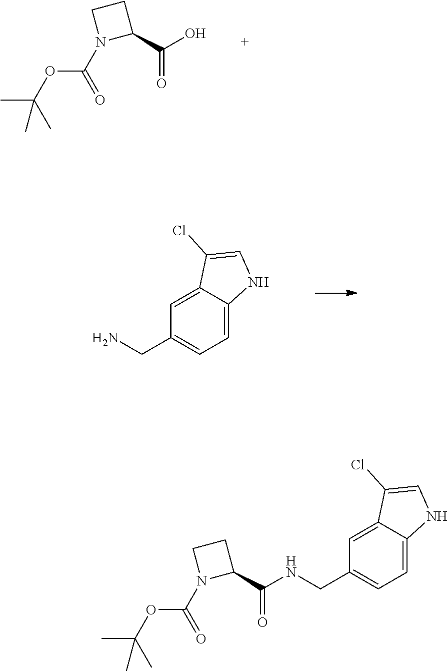 [SMILES: CC(C)(C)OC(=O)N1CC[C@H]1C(=O)O ; NCc1ccc2[nH]cc(Cl)c2c1 ; CC(C)(C)OC(=O)N1CC[C@H]1C(=O)NCc1ccc2[nH]cc(Cl)c2c1]

tert-Butyl (2S)-2-{[(3-chloro-1H-indol-5-yl)methyl]carbamoyl}azetidine-1-carboxylate was prepared from Boc-L-azetine-2-carboxylic acid and (3-chloro-1H-indol-5-yl)methanamine according to general method C (i).

[M+H]⁺=362.1

### (2S)—N-[(3-Chloro-1H-indol-5-yl)methyl]azetidine-2-carboxamide Hydrochloride

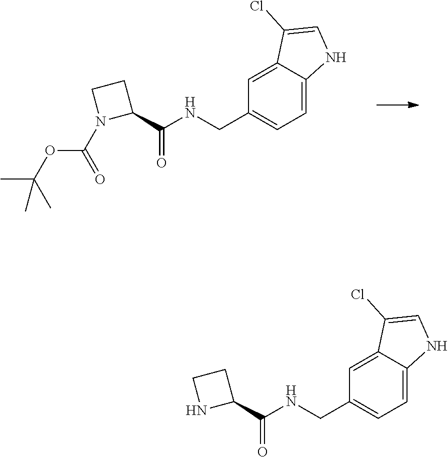 [SMILES: CC(C)(C)OC(=O)N1CC[C@H]1C(=O)NCc1ccc2[nH]cc(Cl)c2c1 ; O=C(NCc1ccc2[nH]cc(Cl)c2c1)[C@@H]1CCN1]

(2S)—N-[(3-Chloro-1H-indol-5-yl)methyl]azetidine-2-carboxamide hydrochloride was prepared from tert-butyl (2S)-2-{[(3-chloro-1H-indol-5-yl)methyl]carbamoyl}azetidine-1-carboxylate according to general method A (i).

[M+H1]=264.1

### tert-Butyl N-[(2R)-1-[(2S)-2-{[(3-chloro-1H-indol-5-yl)methyl]carbamoyl}azetidin-1-yl]-1-oxo-6-(piperidin-1-yl)hexan-2-yl]carbamate

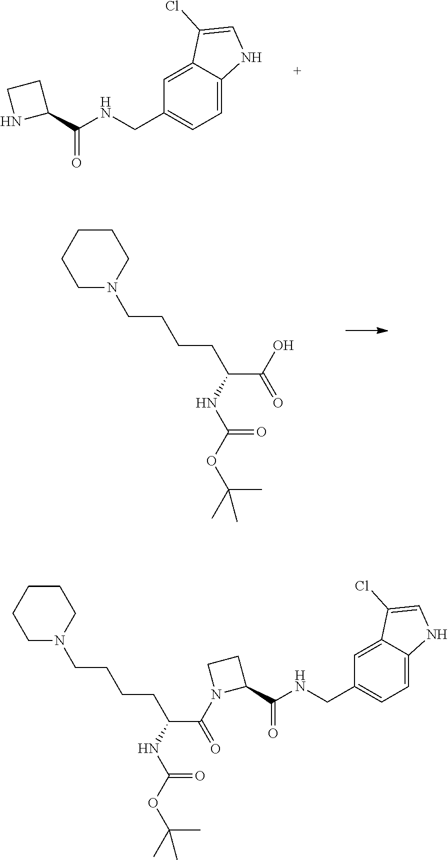 [SMILES: O=C(NCc1ccc2[nH]cc(Cl)c2c1)[C@@H]1CCN1 ; CC(C)(C)OC(=O)N[C@H](CCCCN1CCCCC1)C(=O)O ; CC(C)(C)OC(=O)N[C@H](CCCCN1CCCCC1)C(=O)N1CC[C@H]1C(=O)NCc1ccc2[nH]cc(Cl)c2c1]

tert-Butyl N-[(2R)-1-[(2S)-2-{[(3-chloro-1H-indol-5-yl)methyl]carbamoyl}azetidin-1-yl]-1-oxo-6-(piperidin-1-yl)hexan-2-yl]carbamate was prepared from (2S)—N-[(3-chloro-1H-indol-5-yl)methyl]azetidine-2-carboxamide and (2R)-2-[(tert-butoxycarbonyl)amino]-6-(piperidin-1-yl)hexanoic acid according to general method C (i).

[M+H]⁺=560.4

### (2S)-1-[(2R)-2-Amino-6-(piperidin-1-yl)hexanoyl]-N-[(3-chloro-1H-indol-5-yl)methyl]azetidine-2-carboxamide Hydrochloride

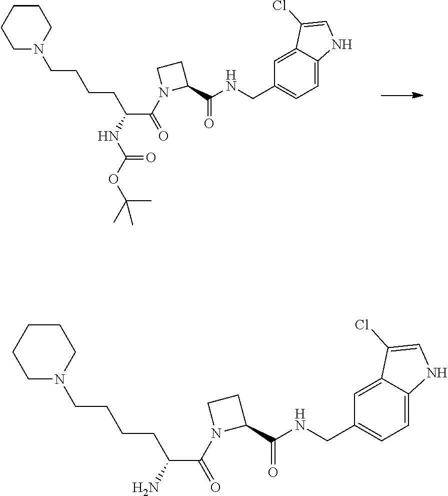 [SMILES: CC(C)(C)OC(=O)N[C@H](CCCCN1CCCCC1)C(=O)N1CC[C@H]1C(=O)NCc1ccc2[nH]cc(Cl)c2c1 ; N[C@H](CCCCN1CCCCC1)C(=O)N1CC[C@H]1C(=O)NCc1ccc2[nH]cc(Cl)c2c1]

(2S)-1-[(2R)-2-Amino-6-(piperidin-1-yl)hexanoyl]-N-[(3-chloro-1H-indol-5-yl)methyl]azetidine-2-carboxamide hydrochloride was prepared from tert-butyl N-[(2R)-1-[(2S)-2-{[(3-chloro-1H-indol-5-yl)methyl]carbamoyl}azetidin-1-yl]-1-oxo-6-(piperidin-1-yl)hexan-2-yl]carbamate according to general method A (i).

[M+H]⁺=460.2

### (2S)—N-[(3-Chloro-1H-indol-5-yl)methyl]-1-[(2R)-2-(isopropylamino)-6-(piperidin-1-yl)hexanoyl]azetidine-2-carboxamide

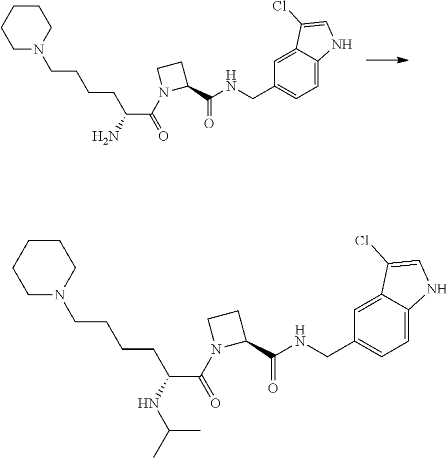 [SMILES: N[C@H](CCCCN1CCCCC1)C(=O)N1CC[C@H]1C(=O)NCc1ccc2[nH]cc(Cl)c2c1 ; CC(C)N[C@H](CCCCN1CCCCC1)C(=O)N1CC[C@H]1C(=O)NCc1ccc2[nH]cc(Cl)c2c1]

(2S)—N-[(3-Chloro-1H-indol-5-yl)methyl]-1-[(2R)-2-(isopropylamino)-6-(piperidin-1-yl)hexanoyl]azetidine-2-carboxamide was prepared from (2S)-1-[(2R)-2-Amino-6-(piperidin-1-yl)hexanoyl]-N-[(3-chloro-1H-indol-5-yl)methyl]azetidine-2-carboxamide hydrochloride and acetone according to general method F.

[M+H]⁺=502.4

¹H NMR (400 MHz, DMSO): 0.92-0.81 (6H, m), 1.49-1.20 (12H, m), 2.31-2.00 (6H, m), 2.52-2.49 (3H, m), 2.93-2.88 (1H, m), 3.09 (1H, t, J=5.8 Hz), 3.86-3.80 (1H, m), 4.21-4.04 (1H, m), 4.45-4.38 (2H, m), 4.73-4.68, 4.90-4.85 (1H, m), 7.13-7.06 (1H, m), 7.37 (2H, dd, J=8.3, 12.9 Hz), 7.51-7.48 (1H, m), 8.45-8.40, 8.80-8.75 (1H, m), 11.32 (1H, d, J=6.3 Hz).

### Example 12.06

### (2R)—N-[(1S)-1-{[(1-Aminoisoquinolin-6-yl)methyl]carbamoyl}-2-(naphthalen-1-yl)ethyl]-2-(isopropylamino)-6-(piperidin-1-yl)hexanamide

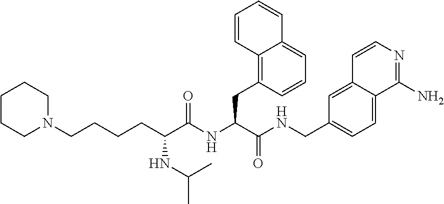 [SMILES: CC(C)N[C@H](CCCCN1CCCCC1)C(=O)N[C@@H](Cc1cccc2ccccc12)C(=O)NCc1ccc2c(N)nccc2c1]

### tert-Butyl N-[(1S)-1-{[(1-aminoisoquinolin-6-yl)methyl]carbamoyl}-2-(naphthalen-1-yl)ethyl]carbamate

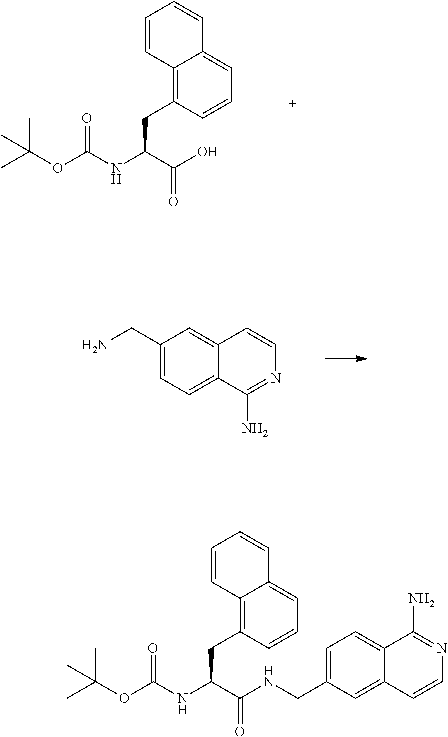 [SMILES: CC(C)(C)OC(=O)N[C@@H](Cc1cccc2ccccc12)C(=O)O ; NCc1ccc2c(N)nccc2c1 ; CC(C)(C)OC(=O)N[C@@H](Cc1cccc2ccccc12)C(=O)NCc1ccc2c(N)nccc2c1]

According to general method C (ii), the title compound was prepared from N-Boc-3-(2-napthyl)-L-alanine and 6-aminomethyl-isoquinolin-1-amine. (141 mg, 41% yield).

[M+H]⁺=471.4

### (2S)-2-Amino-N-[(1-aminoisoquinolin-6-yl)methyl]-3-(naphthalen-1-yl)propanamide Dihydrochloride

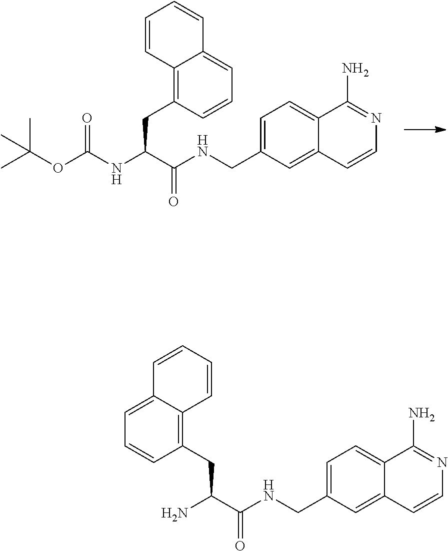 [SMILES: CC(C)(C)OC(=O)N[C@@H](Cc1cccc2ccccc12)C(=O)NCc1ccc2c(N)nccc2c1 ; Nc1nccc2cc(CNC(=O)[C@@H](N)Cc3cccc4ccccc34)ccc12]

(2S)-2-Amino-N-[(1-aminoisoquinolin-6-yl)methyl]-3-(naphthalen-1-yl)propanamide dihydrochloride was prepared from tert-butyl N-[(1S)-1-{[(1-aminoisoquinolin-6-yl)methyl]carbamoyl}-2-(naphthalen-1-yl)ethyl]carbamate according to general method A (i). (136 mg, 97% yield).

[M+H]⁺=371.3

### Methyl (2R)-2-amino-6-(piperidin-1-yl)hexanoate Dihydrochloride

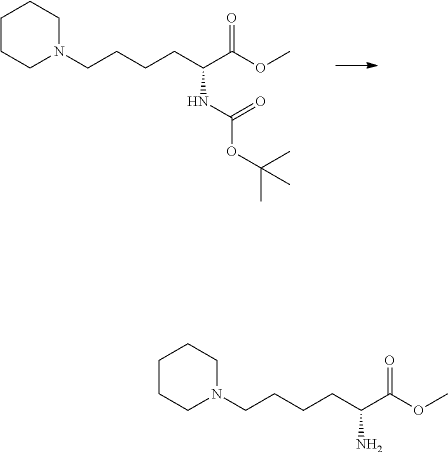 [SMILES: COC(=O)[C@@H](CCCCN1CCCCC1)NC(=O)OC(C)(C)C ; COC(=O)[C@H](N)CCCCN1CCCCC1]

Methyl (2R)-2-amino-6-(piperidin-1-yl)hexanoate dihydrochloride was prepared from methyl (2R)-2-[(tert-butoxycarbonyl)amino]-6-(piperidin-1-yl)hexanoate according to general method A (i). (541 mg, 100% yield).

[M+H]⁺=229.4

### Methyl (2R)-2-(isopropylamino)-6-(piperidin-1-yl)hexanoate

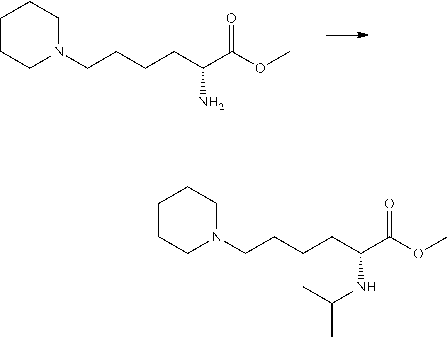 [SMILES: COC(=O)[C@H](N)CCCCN1CCCCC1 ; COC(=O)[C@@H](CCCCN1CCCCC1)NC(C)C]

Methyl (2R)-2-(isopropylamino)-6-(piperidin-1-yl)hexanoate was prepared from methyl (2R)-2 methyl (2R)-2-amino-6-(piperidin-1-yl)hexanoate dihydrochloride and acetone according to general method F (443 mg, 82% yield).

[M+H]⁺=271.4

### (2R)-2-(isopropylamino)-6-(piperidin-1-yl) Hexanoic Acid

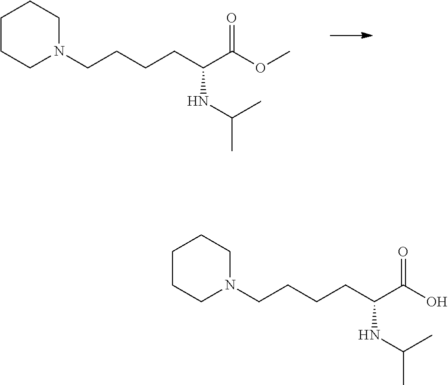 [SMILES: COC(=O)[C@@H](CCCCN1CCCCC1)NC(C)C ; CC(C)N[C@H](CCCCN1CCCCC1)C(=O)O]

(2R)-2-(Isopropylamino)-6-(piperidin-1-yl)hexanoic acid was prepared from methyl (2R)-2 methyl (2R)-2-amino-6-(piperidin-1-yl)hexanoate dihydrochloride and acetone according to general method D (i) (443 mg, 82% yield).

[M+H]⁺=271.4

### (2R)—N-[(1S)-1-{[(1-aminoisoquinolin-6-yl)methyl]carbamoyl}-2-(naphthalen-1-yl)ethyl]-2-(isopropylamino)-6-(piperidin-1-yl)hexanamide

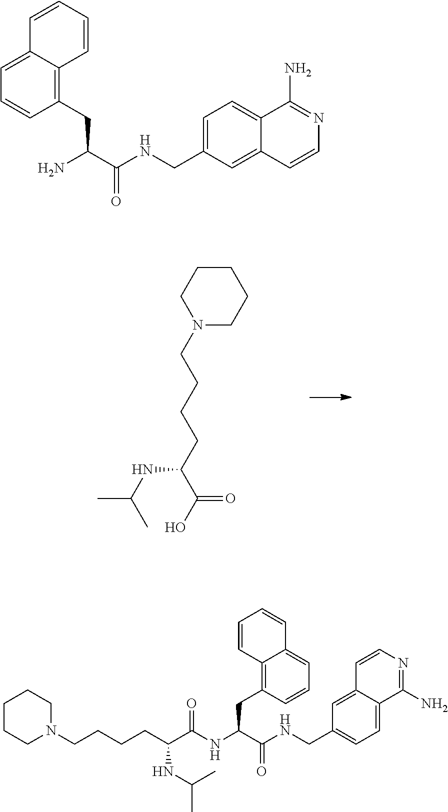 [SMILES: Nc1nccc2cc(CNC(=O)[C@@H](N)Cc3cccc4ccccc34)ccc12 ; CC(C)N[C@H](CCCCN1CCCCC1)C(=O)O ; CC(C)N[C@H](CCCCN1CCCCC1)C(=O)N[C@@H](Cc1cccc2ccccc12)C(=O)NCc1ccc2c(N)nccc2c1]

(2R)—N-[(1S)-1-{[(1-Aminoisoquinolin-6-yl)methyl]carbamoyl}-2-(naphthalen-1-yl)ethyl]-2-(isopropylamino)-6-(piperidin-1-yl)hexanamide was prepared from (2S)-2-amino-N-[(1-aminoisoquinolin-6-yl)methyl]-3-(naphthalen-1-yl)propanamide dihydrochloride and (2R)-2-(isopropylamino)-6-(piperidin-1-yl)hexanoic acid according to general method C (i). 15 mg, 34% yield.

[M+H]⁺=609.3

1H NMR (DMSO): 0.81 (6H, dd, J=20.0, 6.1 Hz), 1.01-1.11 (2H, m), 1.21-1.28 (4H, m), 1.29-1.34 (3H, m), 1.41-1.45 (5H, m), 2.03 (2H, t, J=7.2 Hz), 2.20 (4H, s), 2.34-2.41 (1H, m), 2.97 (1H, t, J=6.8 Hz), 3.47-3.52 (1H, m), 4.40 (2H, d, J=5.8 Hz), 4.76 (1H, q, J=5.9 Hz), 6.71 (2H, s), 6.78 (1H, d, J=5.8 Hz), 7.23 (1H, dd, J=8.6, 1.4 Hz), 7.37-7.40 (3H, m), 7.50-7.59 (2H, m), 7.75 (1H, d, J=5.8 Hz), 7.80 (1H, dd, J=6.6, 2.4 Hz), 7.92 (1H, d, J=7.0 Hz), 8.08 (1H, d, J=8.6 Hz), 8.27 (2H, t, J=7.7 Hz), 8.61 (1H, t, J=5.7 Hz)

### Example 17.09

### (2S)—N-[(1-aminoisoquinolin-6-yl)methyl]-1-[(2R)-2-(isopropylamino)-4-phenylbutanoyl]azetidine-2-carboxamide

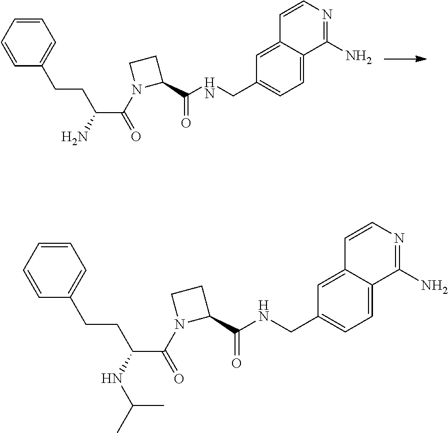 [SMILES: Nc1nccc2cc(CNC(=O)[C@@H]3CCN3C(=O)[C@H](N)CCc3ccccc3)ccc12 ; CC(C)N[C@H](CCc1ccccc1)C(=O)N1CC[C@H]1C(=O)NCc1ccc2c(N)nccc2c1]

Following general method F, (2S)-1-[(2R)-2-amino-4-phenylbutanoyl]-N-[(1-aminoisoquinolin-6-yl)methyl]azetidine-2-carboxamide (67 mg, 0.16 mmol) was dissolved in dry DMF (1 mL), acetone 1.2 μL, 0.16 mmol) was added followed by acetic acid (1.8 μL, 0.03 mmol) and stirred for 60 min. Sodium triacetoxyborohydride (170 mg, 0.8 mmol) was added portionwise over 10 min the suspension was stirred for 24 hrs. The reaction mixture was carefully quenched with water and diluted with DCM. The acidic aqueous was separated and washed with DCM (2×20 mL). To the aqueous was then added Na₂CO₃ until the solution reached a basic pH and was then washed with 10% IPA in CHCl₃ (6×25 mL). The combine organics were dried over sodium sulfate and concentrated in vacuo. The crude material was purified by flash chromatography (10% MeOH in DCM) to afford the desired product as a colourless oil (33 mg, 45% yield).

[M+H]⁺=460.5

1H NMR (d6-DMSO) δ: 0.98-0.89 (6H, m), 1.83-1.63 (2H, m), 2.23-2.10 (1H, m), 2.78-2.60 (3H, m), 3.20-3.00 (1H, m), 4.19-3.90 (2H, m), 4.56-4.38 (3H, m), 4.74 (1H, dd, J=5.4, 8.9 Hz), 6.87-6.78 (3H, m), 7.38-6.96 (61H, m), 7.59-7.54 (1H, m), 7.77 (1H, d, J=5.8 Hz), 8.16-8.10 (1H, m), 8.96-8.58 (1H, m).

### Example 27.05

### (2R)—N-[(1S)-1-[({5-chloro-7H-pyrrolo[2,3-b]pyridin-3-yl}methyl)carbamoyl]-2-(3,4-difluorophenyl)ethyl]-2-(cyclohexylamino)-6-(piperidin-1-yl)hexanamide

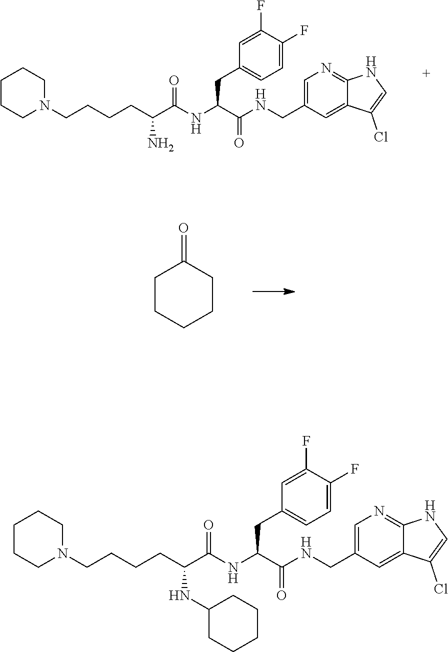 [SMILES: N[C@H](CCCCN1CCCCC1)C(=O)N[C@@H](Cc1ccc(F)c(F)c1)C(=O)NCc1cnc2[nH]cc(Cl)c2c1 ; O=C1CCCCC1 ; O=C(NCc1cnc2[nH]cc(Cl)c2c1)[C@H](Cc1ccc(F)c(F)c1)NC(=O)[C@@H](CCCCN1CCCCC1)NC1CCCCC1]

In a modification to general method F, (2R)-2-amino-N-[(1S)-1-[({5-chloro-7H-pyrrolo[2,3-b]pyridin-3-yl}methyl)carbamoyl]-2-(3,4-difluorophenyl)ethyl]-6-(piperidin-1-yl)hexanamide (70 mg, 0.12 mmol) was dissolved in dry methanol (5 mL), cyclohexanone (150 μL, 1.25 mmol) was added followed by acetic acid (3.6 μL, 0.06 mmol) and stirred for 60 min. Polymer supported sodium cyanoborohydride (249 mg, 0.5 mmol) was added and the suspension was stirred for 18 hrs. The solid was filtered off and washed with methanol (5 mL) and the combined filtrates were concentrated in vacuo. The crude material was purified by flash chromatography (10% MeOH in DCM) to afford the desired product as a colourless oil (31 mg, 38% yield).

[M+H]⁺=643.5

1H NMR (d6-DMSO) δ: 0.82 (2H, q, J=10.2 Hz), 1.10-0.94 (6H, m), 1.49-1.21 (14H, m), 1.76-1.71 (1H, m), 2.07-1.97 (3H, m), 2.21-2.17 (4H, m), 2.83 (1H, dd, J=10.0, 13.8 Hz), 3.03-2.93 (2H, m), 4.47-4.35 (2H, m), 4.63-4.55 (1H, m), 7.04-7.01 (1H, m), 7.30-7.20 (2H, m), 7.66 (1H, s), 7.80-7.78 (1H, m), 8.11-8.07 (1H, m), 8.21 (1H, d, J=2.0 Hz), 8.53-8.47 (1H, m), 11.96-11.93 (1H, m).

### Example 39.06

### (2S)—N-[(1-Aminoisoquinolin-6-yl)methyl]-2-{2-[(2R,6S)-2,6-dimethylpiperidin-1-yl]acetamido}-3-hydroxybutanamide

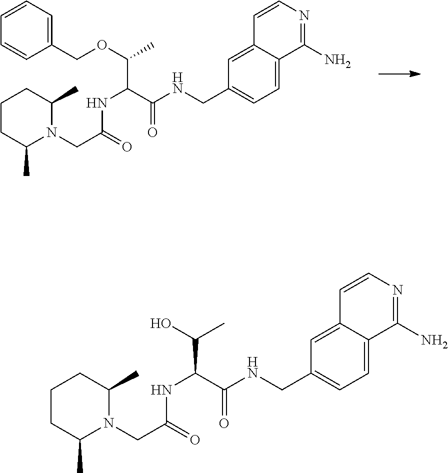 [SMILES: C[C@@H]1CCC[C@H](C)N1CC(=O)NC(C(=O)NCc1ccc2c(N)nccc2c1)[C@@H](C)OCc1ccccc1 ; CC(O)[C@H](NC(=O)CN1[C@H](C)CCC[C@@H]1C)C(=O)NCc1ccc2c(N)nccc2c1]

(2S)—N-[(1-aminoisoquinolin-6-yl)methyl]-3-(benzyloxy)-2-{2-[(2R,6S)-2,6-dimethylpiperidin-1-yl]acetamido}butanamide (150 mg, 0.29 mmol) was dissolved in methanol (50 mL). The solution was hydrogenated over 10% Pd/C (100 mg) at atmospheric pressure for 5 hrs after which time the catalyst was filtered off and washed with methanol (100 mL), the combined filtrates were evaporated in vacuo.

The residue was purified by Prep HPLC, (0 to 60% (0.1% TFA/MeCN) in (0.1% TFA/H2O)) over 35 min at 20 mL/min. The product was freeze dried in MeCN/water to give a white solid identified as the title compound (14 mg, 7% yield).

[M+H]⁺=428.3

1H NMR (d6-DMSO) δ: 1.11-1.19 (6H, m), 1.24-1.29 (4H, m), 1.64-1.78 (4H, m), 2.33 (3H, d, J=1.7 Hz), 2.54 (3H, d, J=1.7 Hz), 4.19-4.34 (2H, m), 4.52 (2H, d, J=5.2 Hz), 6.95 (1H, s), 7.08 (1H, s), 7.20 (1H, s), 6.67-7.72 (2H, m), 7.77 (1H, s), 8.44-8.52 (1H, m), 8.59-8.65 (1H, m), 8.90 (1H, s).

TABLE 24
¹H NMR data of examples (solvent d6 DMSO unless otherwise indicated)

| Example No. | NMR write-up |
|---|---|
| 0.01 | 2.14 (3H, s), 2.16 (3H, s), 2.91-2.97 (1H, m), 3.08-3.12 (1H, m), 4.42 (2H, d, J = |
| | 5.8 Hz), 4.72-4.77 (1H, m), 5.65 (1H, d, J = 1.7 Hz), 6.82 (1H, d, J = 5.9 Hz), 6.90 |
| | (2H, s), 7.09-7.11 (1H, m), 7.26-7.34 (4H, m), 7.49 (1H, s), 7.70 (1H, d, J = 5.6 Hz), |
| | 8.14 (1H, d, J = 8.6 Hz), 8.72 (1H, t, J = 5.9 Hz), 10.89 (1H, s) |
| 0.02 | 1.11 (3H, d, J = 6.7 Hz), 1.21 (3H, d, J = 6.7 Hz), 1.41-1.47 (1H, m), 1.70-1.76 (4H, |
| | m), 2.80-2.86 (2H, m), 3.11-3.15 (1H, m), 3.28 (1H, d, J = 12.2 Hz), 3.39-3.42 |
| | (1H, m), 3.88 (1H, t, J = 9.6 Hz), 4.50-4.54 (2H, m), 4.68-4.74 (1H, m), 7.07-7.09 |
| | (1H, m), 7.17 (1H, d, J = 7.0 Hz), 7.29-7.36 (2H, m), 7.61 (1H, dd, J = 1.4, 8.6 Hz), |
| | 7.69 (1H, d, J = 7.0 Hz), 7.74 (1H, s), 8.49 (1H, d, J = 8.7 Hz), 9.09 (3H, d, J = 8.4 Hz), |
| | 9.38 (1H, br.s), 13.36 (1H, br.s). |
| 0.19 | 1.85-2.01(1H, m), 2.11-2.16 (2H, m), 2.74 (6H, s), 2.88 (1H, q, J = 11.0 Hz), 3.21 |
| | (1H, t, J = 9.5 Hz), 3.73 (1H, br.s), 4.50-4.60 (2H, m), 4.72-4.78 (1H, m), 6.95 (1H, |
| | d, J = 7.1 Hz), 7.16-7.27 (5H, m), 7.36-7.41 (1H, m), 7.61 (1H, d, J = 1.3 Hz), 7.64 |
| | (1H, d, J = 1.3 Hz), 7.68 (1H, d, 7.0 Hz), 7.75 (1H, s), 8.48 (1H, d, J = 8.6 Hz), 8.99 |
| | (3H, t, J = 5.5 Hz), 9.24 (1H, br.s), 9.96(1H, br.s), 13.40 (1H, br.s) |
| 0.64 | 1.40 (6H, d, J = 6.9 Hz), 3.22 (2H, d, J = 7.2 Hz), 3.76 (1H, sept, J = 6.9 Hz), 4.37- |
| | 4.53 (2H, m), 4.86 (1H, dt, J = 8.6, 7.2 Hz), 6.74 (2H, s), 6.82 (1H, d, J = 6.0 Hz), 7.09- |
| | 7.15 (1H, m), 7.27 (1H, dt, J = 10.9, 8.5 Hz), 7.32-7.42 (2H, m), 7.51 (1H, s), 7.66 |
| | (1H, s), 7.76 (1H, d, J = 5.8 Hz), 8.13 (1H, d, J = 8.6 Hz), 8.77 (1H, t, J = 5.9 Hz), 8.86 |
| | (1H, s), 9.23 (1H, d, J = 8.7 Hz). |
| 0.87 | 1.24 (6H, d, J = 7.0 Hz), 3.06-3.26 (3H, m), 4.36-4.51 (2H, m), 4.82 (1H, td, J = 8.8, |
| | 5.3 Hz), 6.73 (2H, s), 6.81 (1H, d, J = 5.4 Hz), 7.05-7.12 (1H, m), 7.21-7.36 (3H, m), |
| | 7.48 (1H, d, J = 1.7 Hz), 7.76 (1H, d, J = 5.8 Hz), 7.86 (1H, d, J = 1.3 Hz), 8.12 (1H, d, |
| | J = 8.6 Hz), 8.77 (1H, t, J = 5.9 Hz), 9.00 (1H, d, J = 8.6 Hz), 9.23 (1H, d, J = 1.3 Hz) |
| 0.88 | 1.24 (9H, s), 2.98 (1H, dd, J = 13.7, 10.4 Hz), 3.16 (1H, dd, J = 13.7, 4.8 Hz), 3.90 |
| | (3H, s), 4.34-4.52 (2H, m), 4.69 (1H, ddd, J = 10.4, 8.4, 4.8 Hz), 6.78-6.83 (2H, m), |
| | 6.88 (2H, s), 7.11-7.19 (1H, m), 7.26-7.44 (3H, m), 7.50 (1H, s), 7.75 (1H, d, J = 5.9 |
| | Hz), 8.15 (1H, d, J = 8.6 Hz), 8.61 (1H, d, J = 8.4 Hz), 8.73 (1H, t, J = 6.0 Hz) |
| 0.89 | 1.04 (6H, dd, J = 6.8, 2.7 Hz), 2.81-2.99 (2H, m), 3.19 (1H, dd, J = 13.8, 4.5 Hz), |
| | 4.39-4.57 (2H, m), 4.85 (1H, ddd, J = 10.9, 8.5, 4.5 Hz), 6.86 (1H, d, J = 5.8 Hz), 6.94 |
| | (2H, s), 7.14-7.19 (1H, m), 7.29-7.43 (3H, m), 7.55 (1H, s), 7.76 (1H, d, J = 5.9 Hz), |
| | 8.17 (1H, d, J = 8.6 Hz), 8.54 (1H, s), 8.77 (1H, t, J = 6.0 Hz), 9.06 (1H, d, J = 8.5 Hz), |
| | 9.13 (1H, s) |
| 0.90 | 1.19 (6H, dd, J = 6.9, 2.8 Hz), 2.86 (1H, hept, J = 6.9 Hz), 2.98 (1H, dd, J = 13.7, 10.5 |
| | Hz), 3.15 (1H, dd, J = 13.7, 4.7 Hz), 3.90 (3H, s), 4.36-4.52 (2H, m), 4.68 (1H, ddd, |
| | J = 10.5, 8.3, 4.7 Hz), 6.71-6.78 (1H, m), 6.84 (1H, d, J = 5.7 Hz), 7.04 (2H, s), 7.12- |
| | 7.19 (1H, m), 7.26-7.44 (3H, m), 7.52 (1H, s), 7.75 (1H, d, J = 6.0 Hz), 8.17 (1H, d, |
| | J = 8.6 Hz), 8.62 (1H, d, J = 8.4 Hz), 8.74 (1H, t, J = 6.0 Hz) |
| 0.91 | 1.35 (6H, dd, J = 6.9, 1.2 Hz), 3.06-3.18 (2H, m), 3.27-3.38 (1H, m), 4.44 (2H, d, |
| | J = 5.9 Hz), 4.78 (1H, td, J = 8.3, 6.0 Hz), 6.84 (1H, d, J = 6.0 Hz), 6.97 (2H, s), 7.03- |
| | 7.10 (1H, m), 7.22-7.38 (3H, m), 7.50 (1H, s), 7.75 (1H, d, J = 5.9 Hz), 8.14 (1H, s), |
| | 8.16 (1H, d, J = 8.6 Hz), 8.21 (1H, d, J = 8.5 Hz), 8.78 (1H, t, J = 6.0 Hz) |
| 0.92 | 1.28 (6H, d, J = 6.9 Hz), 3.00 (1H, dd, J = 13.7, 10.4 Hz), 3.12-3.24 (2H, m), 4.47 |
| | (2H, d, J = 6.0 Hz), 4.77 (1H, ddd, J = 10.4, 8.2, 4.7 Hz), 6.93 (1H, d, J = 6.2 Hz), 7.12- |
| | 7.20 (1H, m), 7.26-7.55 (5H, m), 7.58 (1H, d, J = 1.6 Hz), 7.73 (1H, d, J = 6.2 Hz), |
| | 8.25 (1H, d, J = 8.6 Hz), 8.78 (1H, t, J = 5.9 Hz), 9.01-9.07 (3H, m) |
| 0.93 | 3.22 (2H, d, J = 7.3 Hz), 4.38-4.52 (2H, m), 4.79-4.89 (1H, m), 6.77 (2H, s), 6.83 |
| | (1H, d, J = 5.7 Hz), 7.08-7.16 (1H, m), 7.27 (1H, dt, J = 10.9, 8.5 Hz), 7.31-7.40 (2H, |
| | m), 7.51 (1H, s), 7.76 (1H, d, J = 5.8 Hz), 7.79 (1H, d, J = 6.9 Hz), 8.13 (1H, d, J = 8.6 |
| | Hz), 8.79 (1H, t, J = 6.0 Hz), 8.86 (1H, s), 9.24 (1H, d, J = 8.7 Hz), 9.56 (1H, d, J = 7.0 |
| | Hz) |
| 0.94 | 1.32 (6H, dd, J = 8.6, 6.8 Hz), 2.98 (1H, dd, J = 13.6, 10.5 Hz), 3.15 (1H, dd, J = 13.6, |
| | 4.8 Hz), 4.38-4.50 (2H, m), 4.72 (1H, ddd, J = 10.5, 8.3, 4.8 Hz), 5.03 (1H, hept, J = |
| | 6.8 Hz), 6.41 (1H, d, J = 9.5 Hz), 6.74-6.92 (3H, m), 7.11-7.19 (1H, m), 7.25-7.44 |
| | (3H, m), 7.50 (1H, s), 7.76 (1H, d, J = 5.8 Hz), 7.81 (1H, dd, J = 9.5, 2.5 Hz), 8.14 (1H, |
| | d, J = 8.6 Hz), 8.21 (1H, d, J = 2.5 Hz), 8.61 (1H, d, J = 8.3 Hz), 8.73 (1H, t, J = 6.0 Hz) |
| 0.95 | 1.29 (6H, dd, J = 6.2, 3.1 Hz), 3.01 (1H, dd, J = 13.7, 10.6 Hz), 3.17 (1H, dd, J = 9.8, |
| | 4.7 Hz), 4.38-4.51 (2H, m), 4.74 (1H, ddd, J = 10.6, 8.3, 4.5 Hz), 5.26 (1H, hept, J = |
| | 6.2 Hz), 6.70-6.84 (3H, m), 7.10-7.20 (2H, m), 7.24 (1H, dd, J = 5.3, 1.5 Hz), 7.26- |
| | 7.44 (3H, m), 7.49 (1H, s), 7.76 (1H, d, J = 5.8 Hz), 8.13 (1H, d, J = 8.6 Hz), 8.25 (1H, |
| | d, J = 5.3 Hz), 8.71 (1H, t, J = 6.0 Hz), 8.90 (1H, d, J = 8.3 Hz) |
| 0.96 | 1.28 (6H, d, J = 6.8 Hz), 3.00 (1H, dd, J = 13.7, 10.7 Hz), 3.16 (1H, dd, J = 13.7, 4.4 |
| | Hz), 4.34-4.54 (2H, m), 4.70 (1H, ddd, J = 10.7, 8.3, 4.5 Hz), 5.02 (1H, hept, J = 6.8 |
| | Hz), 6.50 (1H, dd, J = 7.2, 2.0 Hz), 6.80 (1H, d, J = 1.9 Hz), 6.84 (1H, d, J = 5.9 Hz), |
| | 6.89 (2H, s), 7.10-7.19 (1H, m), 7.26-7.44 (3H, m), 7.51 (1H, s), 7.76 (1H, d, J = 5.9 |
| | Hz), 7.81 (1H, d, J = 7.2 Hz), 8.15 (1H, d, J = 8.6 Hz), 8.71 (1H, t, J = 6.0 Hz), 8.83 (1H, |
| | d, J = 8.4 Hz) |
| 0.97 | 1.34 (6H, dd, J = 6.9, 3.9 Hz), 3.02 (1H, dd, J = 13.7, 10.3 Hz), 3.23 (1H, dd, J = 13.6, |
| | 5.0 Hz), 1H under water peak, 4.46 (2H, d, J = 5.8 Hz), 4.81 (1H, td, J = 9.6, 5.0 Hz), |
| | 6.84 (3H, d, J = 6.3 Hz), 7.17 (1H, d, J = 5.4 Hz), 7.26-7.44 (3H, m), 7.50 (1H, s), |
| | 7.77 (1H, d, J = 5.8 Hz), 7.86 (1H, d, J = 2.0 Hz), 8.15 (1H, d, J = 8.6 Hz), 8.80 (1H, t, |
| | J = 6.0 Hz), 9.26 (1H, d, J = 8.4 Hz), 9.34 (1H, d, J = 2.0 Hz) |
| 0.98 | 1.32 (6H, dd, J = 6.9, 1.8 Hz), 3.14-3.26 (3H, m), 4.46 (2H, d, J = 5.9 Hz), 4.81 (1H, |
| | td, J = 8.1, 6.0 Hz), 6.77 (2H, s), 6.83 (1H, d, J = 5.8 Hz), 7.09 (1H, dd, J = 8.3, 4.5 Hz), |
| | 7.21-7.39 (3H, m), 7.51 (1H, d, J = 1.6 Hz), 7.77 (1H, d, J = 5.8 Hz), 8.15 (1H, d, J = |
| | 8.6 Hz), 8.68 (1H, d, J = 8.4 Hz), 8.82 (2H, d, J = 9.1 Hz), 8.95 (1H, s) |
| 0.99 | 1.26 (6H, dd, J = 6.9, 1.2 Hz), 3.07 (1H, dt, J = 13.8, 6.9 Hz), 3.12-3.21 (2H, m), |
| | 4.45 (2H, d, J = 5.8 Hz), 4.81 (1H, td, J = 8.4, 5.6 Hz), 6.87 (1H, d, J = 6.2 Hz), 6.98- |
| | 7.13 (3H, m), 7.21-7.41 (3H, m), 7.53 (1H, s), 7.59 (1H, d, J = 5.2 Hz), 7.75 (1H, d, |
| | J = 6.0 Hz), 8.18 (1H, d, J = 8.6 Hz), 8.76-8.86 (3H, m) |
| 3.02 | 0.64 (3H, d, J = 6.0 Hz), 0.87 (3H, t, J = 6.2 Hz), 0.97-1.28 (3H, m), 1.38-1.46 (2H, |
| | m), 1.54-1.57 (2H, m), 1.74-1.77 (1H, m), 2.32-2.33 (1H, m), 2.88 (2H, s), 3.58- |
| | 3.62 (1H, m), 4.35-4.44 (2H, m), 4.72-4.78 (1H, m), 6.73 (2H, s), 6.79 (1H, d, J = |
| | 5.8 Hz), 7.25 (1H, dd, J = 1.4, 8.6 Hz), 7.36 (2H, s), 7.43 (1H, s), 7.50-7.59 (2H, m), |
| | 7.76 (1H, d, J = 5.8 Hz), 7.82 (1H, dd, J = 3.6, 8.5 Hz), 7.91 (1H, d, J = 1.0 Hz), 7.93 |
| | (1H, s), 8.10 (1H, d, J = 8.6 Hz), 8.27 (1H, d, J = 8.4 Hz), 8.71 (1H, t, J = 5.7 Hz) |
| 3.03 | 2.50 (6H, s), 2.83 (1H, dd, J = 3.4, 7.4 Hz), 3.10 (1H, dd, J = 5.0, 13.8 Hz), 3.87 (2H, |
| | q, 5.2 Hz), 4.43-4.55 (2H, m), 4.67-4.72 (1H, m), 7.06-7.09 (1H, m), 7.15 (1H, d, |
| | J = 7.0 Hz), 7.27-7.32 (2H, m), 7.61 (1H, dd, J = 1.4, 8.6 Hz), 7.69 (1H, s), 7.70 (1H, d, |
| | J = 8.4 Hz), 8.48 (1H, d, J = 8.6 Hz), 8.91-8.94 (1H, m), 8.98-9.05 (2H, m), 9.67 (1H, |
| | br.s) |
| 3.04 | 0.77 (3H, d, J = 6.2 Hz), 0.87 (3H, d, J = 6.1 Hz), 1.07-1.13 (2H, m), 1.21-1.28 (2H, |
| | m), 1.44-1.47 (2H, m), 1.56-1.59 (2H, m), 2.32-2.39 (1H, m), 2.91-2.98 (4H, m), |
| | 3.04-3.09 (1H, m), 4.41 (2H, d, J = 5.8 Hz), 4.67-4.71 (1H, m), 6.74 (1H, s), 6.81 |
| | (1H, d, J = 5.9 Hz), 7.03 (1H, d, J = 8.8 Hz), 7.26-7.29 (3H, m), 7.47 (1H, s), 7.76 (1H, |
| | d, J = 5.8 Hz), 7.88 (1H, d, J = 7.5 Hz), 8.11 (1H, d, J = 8.6 Hz), 8.69 (1H, t, J = 5.4 Hz) |
| 3.05 | 0.75 (3H, d, J = 5.7 Hz), 0.88 (3H, d, J = 5.9 Hz), 1.12-1.15 (2H, m), 1.22-1.28 (2H, |
| | m), 1.40-1.49 (2H, m), 1.57-1.60 (2H, m), 2.32-2.34 (1H, m), 2.91-2.98 (2H, m), |
| | 3.09-3.17 (2H, m), 4.35-4.47 (2H, m), 4.69-4.74 (1H, m), 6.76 (1H, s), 6.80 (1H, |
| | d, J = 5.8 Hz), 7.19-7.29 (3H, m), 7.33-7.37 (2H, m), 7.40-7.42 (1H, m), 7.76 (1H, |
| | d, J = 5.8 Hz), 7.91 (1H, br.s), 8.11 (1H, d, J = 8.6 Hz), 8.69 (1H, t, J = 5.5 Hz) |
| 3.06 | 1.33-1.44 (6H, m), 2.20-2.30 (3H, m), 2.67-2.75 (1H, m), 2.91-2.98 (3H, m), |
| | 3.05-3.09 (1H, m), 4.42 (2H, d, J = 5.8 Hz), 4.49 (1H, d, J = 5.8 Hz), 6.75 (1H, s), 6.82 |
| | (1H, d, J = 5.8 Hz), 7.05-7.15 (1H, m), 7.28-7.33 (4H, m), 7.48 (1H, s), 7.77 (1H, d, |
| | J = 5.7 Hz), 7.82 (1H, d, J = 8.6 Hz), 8.13 (1H, d, J = 8.6 Hz), 8.66(1H, t, J = 5.9 Hz) |
| 3.07 | 2.20-2.33 (4H, m), 2.67 (2H, d, J = 1.8 Hz), 2.81-2.97 (1H, m), 3.04-3.09 (1H, m), |
| | 3.49-3.56 (4H, m), 4.42 (2H, d, J = 5.9 Hz), 4.62-4.66 (1H, m), 6.80 (1H, s), 6.83 |
| | (1H, d, J = 5.9 Hz), 7.06-7.08 (1H, m), 7.27-7.34 (4H, m), 7.48 (1H, s), 7.76 (1H, d, |
| | J = 5.8 Hz), 7.89 (1H, d, J = 8.6 Hz), 8.13 (1H, d, J = 8.6 Hz), 8.65(1H, t, J = 5.9 Hz) |
| 3.08 | 1.16 (6H, d, J = 4.6 Hz), 2.53-2.61 (3H, m), 2.83 (1H, dd, J = 9.8, 13.7 Hz), 3.13 |
| | (1H, dd, J = 4.8, 13.7 Hz), 3.23-3.26 (1H, m), 4.46-4.53 (2H, m), 4.69-4.75 (1H, |
| | m), 7.07-7.10 (1H, m), 7.16 (1H, d, J = 7.0 Hz), 7.28-7.35 (2H, m), 7.61 (1H, dd, J = |
| | 1.4, 8.8 Hz), 7.69 (1H, s), 7.72 (1H, d, J = 11.1 Hz), 8.49 (1H, d, J = 8.7 Hz), 8.96-9.01 |
| | (2H, m), 9.19 (2H, s), 9.44 (1H, br.s), 13.57 (1H, br.s) |
| 3.09 | 0.90 (12H, d, J = 4.7 Hz), 2.89-2.95 (4H, m), 3.04-3.08 (2H, m), 4.43 (2H, d, J = 5.4 |
| | Hz), 4.66-4.72 (1H, m), 5.11 (1H, s), 6.86 (1H, d, J = 6.0 Hz), 7.01-7.11 (3H, m), |
| | 7.18-7.27 (2H, m), 7.37 (1H, s), 7.50 (1H, s), 7.76 (1H, d, J = 6.0 Hz), 8.17 (1H, d, J = |
| | 8.6 Hz), 8.76 (1H, br.s) |
| 3.10 | 0.67 (3H, d, J = 6.2 Hz), 0.86 (3H, d, J = 6.2 Hz), 0.97-1.14 (2H, m), 1.22-1.28 (1H, |
| | m), 1.38-1.45 (2H, m), 1.54-1.57 (1H, m), 2.33-2.36 (2H, m), 2.91 (2H, s), 3.18- |
| | 3.32 (2H, m), 4.37-4.48 (2H, m), 4.75-4.80 (1H, m), 6.75 (2H, s), 6.80 (1H, d, J = |
| | 5.9 Hz), 7.29 (1H, dd, J = 8.6, 1.4 Hz), 7.35-7.43 (2H, m), 7.46 (2H, s), 7.76 (1H, d, |
| | J = 5.7 Hz), 7.93-7.98 (3H, m), 8.11 (1H, d, J = 8.6 Hz), 8.81 (1H, t, J = 5.9 Hz). |
| 3.11 | 0.77 (3H, d, J = 6.0 Hz), 0.88 (3H, d, J = 5.8 Hz), 1.00-1.28 (4H, m), 1.45-1.60 (3H, |
| | m), 2.33-2.39 (2H, m), 2.88-2.94 (2H, m), 3.02-3.07 (1H, m), 4.41 (2H, d, J = 5.8 |
| | Hz), 4.63-4.69 (1H, m), 6.77 (2H, s), 6.82 (1H, d, J = 5.8 Hz), 7.18-7.29 (6H, m), |
| | 7.46 (1H, s), 7.77 (1H, d, J = 5.8 Hz), 7.87 (1H, s), 8.12 (1H, d, J = 8.6 Hz), 8.68 (1H, t, |
| | J = 5.6 Hz) |
| 3.12 | 0.79 (3H, d, J = 5.8 Hz), 0.88 (3H, d, J = 5.4 Hz), 0.92-1.29 (4H, m), 1.45-1.60 (3H, |
| | m), 2.33-2.39 (2H, m), 2.88-2.93 (2H, m), 2.98-3.06 (1H, m), 4.41 (2H, d, J = 5.8 |
| | Hz), 4.64-4.69 (1H, m), 6.77 (2H, s), 6.82 (1H, d, J = 5.8 Hz), 7.20 (1H, d, J = 8.4 Hz), |
| | 7.23-7.35 (4H, m), 7.46 (1H, s), 7.77 (1H, d, J = 5.8 Hz), 7.88 (1H, s), 8.13 (1H, d, J = |
| | 8.6 Hz), 8.71 (1H, t, J = 5.8 Hz) |
| 3.13 | 0.79 (3H, d, J = 6.1 Hz), 0.89 (3H, d, J = 5.8 Hz), 1.00-1.29 (4H, m), 1.45-1.60 (3H, |
| | m), 2.33-2.40 (2H, m), 2.88-2.96 (2H, m), 3.04-3.08 (1H, m), 4.43 (2H, d, J = 5.8 |
| | Hz), 4.65-4.71 (1H, m), 6.76 (2H, s), 6.83 (1H, d, J = 5.8 Hz), 7.15-7.17 (1H, m), |
| | 7.23-7.32 (4H, m), 7.48 (1H, s), 7.77 (1H, d, J = 5.8 Hz), 7.90 (1H, s), 8.13 (1H, d, J = |
| | 8.6 Hz), 8.71 (1H, t, J = 5.5 Hz) |
| 3.14 | 0.78 (3H, d, J = 5.7 Hz), 0.87 (3H, d, J = 4.9 Hz), 1.07-1.13 (2H, m), 1.24-1.28 (2H, |
| | m), 1.40-1.47 (2H, m), 1.57-1.60 (2H, m), 2.32-2.33 (2H, m), 2.87-2.92 (2H, m), |
| | 3.00-3.06 (1H, m), 4.41 (2H, d, J = 5.7 Hz), 4.64-4.68 (1H, m), 6.78 (1H, s), 6.81 |
| | (1H, d, J = 5.8 Hz), 7.02-7.06 (3H, m), 7.19-7.23 (2H, m), 7.30 (1H, dd, J = 1.4, 8.6 |
| | Hz), 7.46 (1H, s), 7.76 (1H, d, J = 5.8 Hz), 8.12 (1H, d, J = 8.6 Hz), 8.69 (1H, br.s) |
| 3.15 | 0.96(3H, d, J = 6.2 Hz), 1.01 (3H, d, J = 6.24 Hz), 1.15-1.36 (4H, m), 1.48-1.63(4H, |
| | m), 1.82-2.02 (3H, m), 2.89-2.91 (1H, m), 2.95-3.08 (2H, m), 4.37-4.50 (3H, m), |
| | 6.72(2H, s), 6.80 (1H, d, J = 5.8 Hz), 7.10-7.19 (3H, m), 7.24-7.28 (2H, m), 7.31- |
| | 7.34 (1H, m), 7.50(1H, s), 7.76(1H, d, J = 5.8 Hz), 8.09 (1H, d, J = 8.8 Hz), 8.13 (1H, d, |
| | J = 8.6 Hz), 8.72 (1H, t, J = 5.9 Hz) |
| 3.16 | 0.79-0.90 (2H, m), 0.94 (3H, d, J = 6.2 Hz), 0.97 (3H, d, J = 6.3 Hz), 1.00-1.35 (8H, |
| | m), 1.44-1.78 (10H, m), 2.87-3.08 (3H, m), 4.38-4.51 (3H, m), 6.73 (2H, s), 6.80 |
| | (1H, d, J = 5.8 Hz), 7.30 (1H, dd, J = 8.6, 1.5 Hz), 7.47 (1H, s), 7.76 (1H, d, J = 5.8 Hz), |
| | 7.87 (1H, d, J = 9.1 Hz), 8.12 (1H, d, J = 8.6 Hz), 8.67 (1H, t, J = 5.9 Hz) |
| 3.17 | 0.78 (3H, d, J = 6.2 Hz), 0.88 (3H, t, J = 6.3 Hz), 0.98-1.31 (3H, m), 1.44-1.60 (3H, |
| | m), 2.45-2.49 (2H, m), 2.86-3.07 (4H, m), 4.42 (2H, d, J = 5.8 Hz), 4.62-4.70 (1H, |
| | m), 6.73 (2H, s), 6.81 (1H, d, J = 5.8 Hz), 7.02-7.04 (1H, m), 7.22-7.31 (3H, m), |
| | 7.46 (1H, s), 7.77 (1H, d, J = 5.7 Hz), 7.90 (1H, d, J = 8.6 Hz), 8.12 (1H, d, J = 8.6 Hz), |
| | 8.69(1H, t, J = 5.8 Hz) |
| 3.18 | 0.89 (3H, s), 0.92 (3H, s), 1.55-1.74 (4H, m), 2.56-2.62 (1H, m), 2.87 (2H, d, J = |
| | 10.6 Hz), 2.91-2.97 (1H, m), 3.03-3.08 (1H, m), 3.18 (1H, br.s), 4.42 (2H, d, J = 5.8 |
| | Hz), 4.59-4.65 (1H, m), 6.73 (2H, s), 6.82 (1H, d, J = 5.8 Hz), 7.00-7.06 (1H, m), |
| | 7.21-7.33 (3H, m), 7.48 (1H, s), 7.77 (1H, d, J = 5.8 Hz), 7.80 (1H, s), 8.12 (1H, d, J = |
| | 8.6 Hz), 8.66 (1H, t, J = 5.9 Hz) |
| 3.19 | 1.13 (3H, d, J = 6.7 Hz), 1.19 (3H, d, J = 6.3 Hz), 1.24-1.28 (1H, m), 1.58 (2H, s), |
| | 2.32-2.45 (2H, m), 2.82-2.87 (1H, m), 3.07-3.17 (1H, m), 3.60 (1H, t, J = 6.5 Hz), |
| | 3.78-3.88 (2H, m), 4.48-4.55 (2H, m), 4.65-4.75 (1H, m), 7.10 (1H, s), 7.15 (1H, |
| | d, J = 7.0 Hz), 7.29-7.36 (2H, m), 7.59-7.61 (1H, m), 7.69 (1H, t, J = 7.0 Hz), 8.47 |
| | (1H, d, J = 8.5 Hz), 8.91-8.97 (3H, m), 8.99-9.04 (2H, m), 9.49 (1H, s), 13.92 (1H, s) |
| 3.20 | 0.92 (3H, s), 1.14-1.19 (1H, m), 1.27 (3H, s), 1.57 (2H, s), 2.09-2.14 (2H, m), 2.80- |
| | 2.86 (1H, m), 3.12-3.16 (1H, m), 3.67-3.71 (2H, m), 3.99 (1H, d, J = 15.9 Hz), 4.45- |
| | 4.58 (2H, m), 4.73-4.78 (1H, m), 7.09 (1H, d, J = 4.3 Hz), 7.16 (1H, d, J = 7.0 Hz), |
| | 7.29-7.36 (2H, m), 7.59-7.62 (1H, m), 7.69 (1H, t, J = 6.9 Hz), 8.48 (1H, d, J = 8.6 |
| | Hz), 8.93 (1H, t, J = 5.9 Hz), 8.99-9.04 (2H, m), 9.49 (1H, s), 13.26 (1H, s) |
| 3.21 | 0.70 (3H, d, J = 19.0 Hz), 0.87 (3H, d, J = 11.7 Hz), 1.00-1.23 (2H, m), 1.45-1.54 |
| | (4H, m), 1.76-1.77 (1H, m), 2.32-2.33 (2H, m), 2.49-2.50 (3H, m), 2.98-3.09 |
| | (2H, m), 3.16-3.21 (1H, m), 4.39-4.46 (2H, m), 4.58-4.66 (1H, m), 6.79 (1H, d, J = |
| | 6.0 Hz), 6.89 (1H, s), 6.94 (1H, d, J = 0.7 Hz), 6.97 (1H, d, J = 7.2 Hz), 7.04-7.14 (1H, |
| | m), 7.29-7.35 (2H, m), 7.45 (1H, s), 7.61 (1H, d, J = 7.8 Hz), 7.74 (1H, d, J = 5.8 Hz), |
| | 8.13 (1H, d, J = 8.6 Hz), 8.70 (1H, s) |
| 3.22 | 0.82 (3H, t, J = 7.4 Hz), 0.93-0.99 (6H, m), 1.15-1.35 (4H, m), 1.48-1.77 (6H, m), |
| | 2.89-3.08 (2H, m), 4.33-4.47 (3H, m), 6.73 (2H, s), 6.81 (1H, d, J = 5.8 Hz), 7.31 |
| | (1H, dd, J = 8.6, 1.6 Hz), 7.49 (1H, s), 7.76 (1H, d, J = 5.7 Hz), 7.98 (1H, d, J = 6.4 Hz), |
| | 8.12 (1H, d, J = 8.6 Hz), 8.69 (1H, t, J = 5.9 Hz) |
| 3.42 | 0.80 (3H, d, J = 6.3 Hz), 0.91-0.87 (3H, m), 1.30-1.08 (3H, m), 1.47 (2H, d, J = 11.5 |
| | Hz), 1.61-1.57 (1H, m), 2.43-2.40 (2H, m), 3.06-2.87 (4H, m), 4.43 (2H, d, J = 5.8 |
| | Hz), 4.72-4.65 (1H, m), 6.73 (2H, s), 6.83 (1H, d, J = 5.8 Hz), 7.07-7.02 (1H, m), |
| | 7.34-7.23 (3H, m), 7.48-7.47 (1H, m), 7.78 (1H, d, J = 5.9 Hz), 7.93-7.89 (1H, m), |
| | 8.16-8.12 (1H, m), 8.70 (1H, t, J = 6.0 Hz) |
| 3.101 | 0.81 (3H, d, J = 6.5 Hz), 1.08 (3H, t, J = 6.6 Hz), 1.25-1.31 (2H, m), 1.42-1.54 (2H, |
| | m), 1.59-1.63 (4H, m), 2.78-2.81 (1H, m), 3.03-3.07 (1H, m), 3.34 (2H, br.s), 4.42 |
| | (2H, d, J = 5.7 Hz), 4.70-4.75 (1H, m), 7.04 (1H, br.s), 7.24-7.31 (2H, m), 7.68 (1H, |
| | d, J = 2.9 Hz), 7.79 (1H, d, J = 1.3 Hz), 8.22 (1H, d, J = 1.7 Hz), 8.76-8.81 (2H, m), |
| | 11.96 (1H, s) |
| 3.102 | 0.77 (3H, d, J = 6.2 Hz), 0.87 (3H, t, J = 6.3 Hz), 1.07-1.16 (2H, m), 1.21-1.28 (1H, |
| | m), 1.45 (1H, d, J = 12.4 Hz), 1.56-1.59 (1H, m), 2.33-2.38 (2H, m), 2.88-2.95 |
| | (2H, m), 3.03-3.07 (1H, m), 3.34 (2H, br.s), 4.06-4.20 (2H, m), 4.62-4.68 (1H, m), |
| | 5.85 (2H, s), 6.28 (2H, s), 7.02-7.05 (1H, m), 7.22-7.33 (2H, m), 7.79 (1H, d, J = 5.2 |
| | Hz), 7.86 (1H, d, J = 8.7 Hz), 8.58 (1H, t, J = 5.8 Hz) |
| 3.104 | 0.82 (3H, d, J = 6.5 Hz), 1.10 (3H, t, J = 6.5 Hz), 1.28-1.34 (1H, m), 1.43-1.54 (1H, |
| | m), 1.60-1.70 (4H, m), 2.80 (1H, dd, J = 10.5, 13.7 Hz), 3.11-3.18 (1H, m), 3.34- |
| | 3.36 (1H, m), 3.79-3.96 (2H, m), 4.42 (2H, d, J = 5.8 Hz), 4.69-4.75 (1H, m), 7.04 |
| | (1H, d, J = 8.5 Hz), 7.13 (1H, s), 7.25-7.38 (4H, m), 7.90 (1H, d, J = 8.4 Hz), 8.78 (1H, |
| | s), 8.93 (1H, t, J = 5.8 Hz), 9.02 (1H, d, J = 5.1 Hz), 9.06 (1H, d, J = 11.9 Hz), 9.70 (1H, |
| | s) |
| 3.105 | 0.82 (3H, d, J = 6.6 Hz), 1.11 (3H, t, J = 6.5 Hz), 1.29-1.35 (1H, m), 1.44-1.51 (1H, |
| | m), 1.60-1.71 (4H, m), 2.79-2.84 (1H, m), 3.10-3.16 (1H, m), 3.34-3.35 (1H, m), |
| | 3.60-3.94 (2H, m), 4.42 (2H, d, J = 5.7 Hz), 4.68-4.76 (1H, m), 7.11 (2H, t, J = 0.8 |
| | Hz), 7.25 (1H, s), 7.26-7.38 (2H, m), 7.75 (1H, dd, J = 2.1, 8.1 Hz), 8.80 (1H, s), 8.85 |
| | (1H, t, J = 6.0 Hz), 8.94-9.04 (2H, m), 9.32 (1H, s) |
| 5.101 | 0.40 (3H, t, J = 6.6 Hz), 0.76 (3H, t, J = 7.4 Hz), 0.89-0.94 (2H, m), 1.45-1.47 (1H, |
| | m), 1.60-1.62 (1H, m), 2.13 (6H, s), 2.61-2.63 (1H, m), 2.67-2.68 (1H, m), 2.79- |
| | 2.85 (1H, m), 2.98-3.03 (1H, m), 4.34-4.39 (1H, m), 4.46-4.52 (1H, m), 4.66- |
| | 4.72 (1H, m), 6.79 (1H, d, J = 5.4 Hz), 6.81 (1H, d, J = 5.9 Hz), 7.11 (1H, br.s), 7.27- |
| | 7.34 (2H, m), 7.50 (1H, s), 7.76 (1H, d, J = 5.8 Hz), 8.12 (2H, d, J = 8.6 Hz), 8.62 (1H, |
| | t, J = 5.9 Hz) |
| 5.102 | 0.90 (3H, d, J = 6.5 Hz), 0.92 (3H, d, J = 6.6 Hz), 1.04 (3H, d, J = 7.0 Hz), 2.03 (3H, s), |
| | 2.74-2.75 (1H, m), 2.89-2.95 (1H, m), 3.01-3.06 (1H, m), 3.15-3.17 (1H, m), 4.42 |
| | (2H, d, J = 5.8 Hz), 4.58-4.64 (1H, m), 6.76 (2H, s), 6.81 (1H, d, J = 5.8 Hz), 7.00- |
| | 7.06 (1H, m), 7.21-7.32 (3H, m), 7.47 (1H, s), 7.77 (1H, d, 5.7 Hz), 7.98(1H, br.s), |
| | 8.12 (1H, d, J = 8.6 Hz), 8.64 (1H, t, J = 5.8 Hz) |
| 5.103 | 1.20, 1.38 (3H, 2xd, J = 6.8, 6.8 Hz), 1.76-1.93 (4H, m), 2.80-2.89 (1H, m), 3.01 |
| | (1H, br.s), 3.12-3.17 (1H, m), 3.04-3.58 (3H, m), 3.88 (1H, d, J = 4.7 Hz), 4.45- |
| | 4.58 (2H, m), 4.68-4.76 (1H, m), 7.09 (1H, br.s), 7.16-7.19 (1H, m), 7.29-7.36 |
| | (2H, m), 7.62-7.74 (1H, m), 8.49 (1H, d, J = 8.6 Hz), 8.90-8.95 (1H, m), 9.00-9.11 |
| | (3H, m), 9.94 (1H, s), 13.33 (1H, br.s) |
| 5.104 | 1.01 (3H, d, J = 6.8 Hz), 2.09 (3H, s), 2.66 (2H, s), 2.84-2.98 (1H, m), 4.43 (2H, d, |
| | J = 5.8 Hz), 4.60-4.65 (1H, m), 6.84 (1H, d, J = 5.9 Hz), 6.95 (1H, s), 7.09 (1H, d, J = |
| | 4.1 Hz), 7.26-7.33 (4H, m), 7.34-7.42 (2H, m), 7.49 (1H, s), 7.60 (1H, d, J = 8.4 Hz), |
| | 7.76 (1H, d, J = 5.9 Hz), 8.09 (1H, d, J = 8.5 Hz), 8.17 (1H, d, J = 8.6 Hz), 8.72 (1H, t, |
| | J = 5.7 Hz) |
| 5.105 | 1.16 (3H, d, J = 7.3 Hz), 1.24 (3H, t, J = 6.2 Hz), 1.44 (3H, t, J = 7.0 Hz), 1.61-1.63 |
| | (2H, m), 1.75-1.84 (2H, m), 3.17-3.22 (1H, m), 3.58-3.62 (1H, m), 4.23-4.24 (1H, |
| | m), 4.40-4.50 (3H, m), 4.53-4.79 (1H, m), 6.96-7.08 (1H, m), 7.17 (1H, d, J = 6.9 |
| | Hz), 7.21-7.40 (5H, m), 7.59-7.69 (3H, m), 7.73 (1H, d, J = 6.3 Hz), 8.50 (1H, t, J = |
| | 8.6 Hz), 8.53-9.01 (2H, m), 9.40 (1H, br.s), 12.98 (1H, br.s) |
| 5.106 | 1.16 (3H, d, J = 7.3 Hz), 1.24 (3H, t, J = 6.2 Hz), 1.44 (3H, t, J = 7.0 Hz), 1.61-1.63 |
| | (2H, m), 1.75-1.84 (2H, m), 3.17-3.22 (1H, m), 3.58-3.62 (1H, m), 4.23-4.24 (1H, |
| | m), 4.40-4.50 (3H, m), 4.53-4.79 (1H, m), 6.96-7.08 (1H, m), 7.17 (1H, d, J = 6.9 |
| | Hz), 7.21-7.40 (5H, m), 7.59-7.69 (3H, m), 7.73 (1H, d, J = 6.3 Hz), 8.50 (1H, t, J = |
| | 8.6 Hz), 8.53-9.01 (2H, m), 9.40 (1H, br.s), 12.98 (1H, br.s) |
| 6.01 | 1.35 (9H, s), 1.76-1.96 (2H, m), 2.52-2.67 (2H, m), 3.78 (2H, d, J = 5.7 Hz), 3.89- |
| | 3.94 (1H, m), 4.41-4.48 (2H, m), 6.82 (2H, s), 6.84 (1H, s), 7.14-7.21 (4H, m), 7.24- |
| | 7.28 (2H, m), 7.34 (1H, dd, J = 8.6, 1.5 Hz), 7.51 (1H, s), 7.75 (1H, d, J = 5.8 Hz), |
| | 8.13 (1H, d, J = 8.6 Hz), 8.23 (1H, t, J = 5.5 Hz), 8.32 (1H, t, J = 5.8 Hz) |
| 6.02 | 2.00-2.06 (2H, m), 2.63-2.67 (2H, m), 3.90-3.93 (3H, m), 4.51 (2H, d, J = 5.8 Hz), |
| | 7.10 (1H, s), 7.13-7.33 (5H, m), 7.35 (1H, s), 7.68 (2H, d, J = 7.1 Hz), 7.80 (1H, s), |
| | 8.46 (3H, d, J = 3.2 Hz), 8.56 (1H, d, J = 8.6 Hz), 8.85-8.88 (1H, m), 8.99-9.02 (1H, |
| | m), 9.14 (1H, s), 13.30 (1H, s) |
| 6.03 | 1.26 (9H, s), 1.40 (3H, s), 1.44 (3H, s), 1.74-1.86 (2H, m), 2.54-2.75 (2H, m), 3.89- |
| | 3.94 (1H, m), 4.41-4.52 (2H, m), 7.12-7.31 (7H, m), 7.62-7.68 (2H, m), 7.80 (1H, |
| | s), 8.14 (1H, t, J = 5.8 Hz), 8.20 (1H, s), 8.46 (1H, d, J = 8.6 Hz), 8.90 (2H, s), 12.81 |
| | (1H, s) |
| 6.04 | 0.85 (3H, t, J = 7.3 Hz), 1.34 (9H, s), 1.53-1.64 (1H, m), 1.76-1.92 (3H, m), 2.54- |
| | 2.67 (2H, m), 3.93-3.99 (1H, m), 4.22-4.31 (1H, m), 4.37-4.48 (2H, m), 6.72 (2H, |
| | s), 6.81 (1H, d, J = 5.8 Hz), 7.15-7.20 (4H, m), 7.25-7.29 (2H, m), 7.32 (1H, dd, J = |
| | 8.6, 1.3 Hz), 7.49 (1H, s), 7.75 (1H, d, J = 5.8 Hz), 8.05 (1H, d, J = 7.9 Hz), 8.11 (1H, d, |
| | J = 8.6 Hz), 8.47 (1H, t, J = 5.6 Hz) |
| 6.05 | 0.91 (3H, t, J = 7. 3 Hz), 1.59-1.70 (1H, s), 1.78-1.88 (1H, m), 2.00-2.07 (2H, m), |
| | 2.60-2.72 (2H, m), 3.98-4.05 (1H, m), 4.27-4.33 (1H, m), 4.45-4.55(2H, m), 7.09 |
| | (1H, s), 7.17-7.35 (5H, m), 7.67-7.69 (2H, m), 7.79 (1H, s), 8.48 (3H, d, J = 3.6 Hz), |
| | 8.57 (1H, d, J = 8.6 Hz), 8.93-8.96 (2H, m), 9.14 (2H, s), 13.31 (1H, s) |
| 6.06 | 1.46 (6H, s), 1.94-2.06 (2H, m), 2.55-2.73 (2H, m), 3.89-3.90 (1H, m), 4.38-4.55 |
| | (2H, m), 7.12-7.30 (6H, m), 7.65-7.69 (2H, m), 7.78 (1H, s), 8.42 (3H, s), 8.49- |
| | 8.53 (2H, m), 8.80 (1H, s), 9.01 (2H, s), 13.07 (1H, s) |
| 6.07 | 1.22 (6H, t, J = 6.0 Hz), 2.01-2.05 (2H, m), 2.63-2.67 (2H, m), 3.24 (1H, br.s), 4.53 |
| | (2H, s), 3.96-4.02 (2H, m), 4.54 (2H, d, J = 5.9 Hz), 7.15-7.21 (5H, m), 7.28 (2H, t, |
| | J = 7.5 Hz), 7.66 (1H, s), 7.67 (1H, d, J = 2.8 Hz), 7.80 (1H, s), 8.48 (1H, d, J = 8.6 Hz), |
| | 8.72 (1H, br.s), 8.79 (2H, t, J = 5.9 Hz), 9.04 (2H, t, J = 5.8 Hz) |
| 6.08 | 1.76-1.85 (2H, m), 2.10-2.24 (2H, m), 2.88 (1H, dd, J = 13.7, 10.6 Hz), 3.18 (1H, |
| | dd, J = 13.8, 4.5 Hz), 3.59 (3H, d, J = 6.7 Hz), 3.78 (1H, br.s), 4.49 (1H, dd, J = 16.4, |
| | 5.6 Hz), 4.59 (1H, dd, J = 16.3, 5.9 Hz), 4.76-4.82 (1H, m), 6.95 (2H, d, J = 7.2 Hz), |
| | 7.11-7.33 (9H, m), 7.58 (1H, dd, J = 8.6, 1.3 Hz), 7.67 (1H, d, J = 7.2 Hz), 7.73 (1H, |
| | s), 8.48 (1H, d, J = 8.6 Hz), 8.80 (2H, br.s), 8.98 (1H, t, J = 5.9 Hz), 9.00 (1H, br.s), |
| | 9.09 (1H, d, J = 8.2 Hz), 13.14 (1H, br.s) |
| 6.09 | 1.31 (3H, d, J = 7.0 Hz), 1.86-1.75 (1H, m), 2.02-1.91 (1H, m), 2.67-2.55 (2H, m), |
| | 3.52 (1H, dd, J = 5.5, 7.3 Hz), 4.47-4.36 (3H, m), 6.86-6.79 (3H, m), 7.22-7.18 |
| | (3H, m), 7.32-7.27 (2H, m), 7.36 (1H, dd, J = 1.7, 8.6 Hz), 7.52 (1H, s), 7.77 (1H, d, |
| | J = 5.9 Hz), 8.14 (1H, d, J = 8.5 Hz), 8.25 (2H, s), 8.49 (1H, d, J = 7.2 Hz), 8.65 (1H, dd, |
| | J = 6.0, 6.0 Hz). |
| 6.10 | 1.31 (3H, d, J = 7.1 Hz), 1.90-1.73 (2H, m), 2.28 (3H, s), 2.69-2.55 (2H, m), 3.10 |
| | (1H, t, J = 6.6 Hz), 4.46-4.39 (3H, m), 6.84-6.77 (3H, m), 7.21-7.15 (3H, m), 7.37- |
| | 7.25 (3H, m), 7.52 (1H, s), 7.78-7.75 (1H, m), 8.16-8.11 (1H, m), 8.29-8.25 (1H, |
| | m), 8.62-8.57 (1H, m). |
| 6.11 | 0.99-0.94 (6H, m), 1.31-1.27 (3H, m), 1.73-1.66 (1H, m), 1.86-1.78 (1H, m), |
| | 2.73-2.55 (4H, m), 3.17 (1H, dd, J = 6.0, 7.2 Hz), 4.46-4.38 (3H, m), 6.84-6.75 |
| | (3H, m), 7.20-7.16 (3H, m), 7.36-7.24 (3H, m), 7.52 (1H, s), 7.77-7.75 (1H, m), |
| | 8.16-8.10 (1H, m), 8.27-8.22 (1H, m,), 8.59-8.54 (1H, m). |
| 6.12 | 1.33-1.28 (3H, m), 1.85-1.72 (1H, m), 2.06-1.92 (1H, m), 2.86-2.68 (2H, m), |
| | 3.30-3.26 (1H, m), 4.45-4.36 (3H, m), 6.71 (2H, s), 6.83-6.80 (1H, m), 7.23-7.17 |
| | (2H, m), 7.33 (1H, dd, J = 1.5, 8.7 Hz), 7.52-7.49 (1H, m), 7.78-7.64 (2H, m), 8.15- |
| | 8.11 (1H, m), 8.24 (1H, s), 8.47-8.45 (1H, m), 8.61-8.57 (1H, m). |
| 6.13 | (CD2Cl2) 1.49-1.46 (3H, m), 2.02-1.91 (1H, m), 2.23-2.13 (1H, m), 2.85 (2H, t, J = |
| | 7.5 Hz), 3.44 (1H, dd, J = 5.3, 7.5 Hz), 4.69-4.48 (3H, m), 7.00-6.97 (1H, m), 7.16- |
| | 7.11 (2H, m), 7.44-7.37 (2H, m), 7.65-7.55 (2H, m), 7.79-7.75 (1H, m), 7.90- |
| | 7.87 (1H, m), 8.08-8.02 (1H, m), 8.47-8.44 (1H, m). |
| 6.14 | 1.38-1.31 (6H, m), 1.76 (9H, d, J = 19.4 Hz), 2.88-2.78 (2H, m), 3.01-2.93 (2H, |
| | m), 3.88-3.84 (1H, m), 4.53-4.35 (3H, m), 7.22-7.19 (1H, m), 7.72-7.67 (2H, m), |
| | 7.80 (1H, s), 8.49-8.46 (3H, m), 8.59-8.56 (1H, m), 8.96-8.90 (1H, m), 9.14-9.02 |
| | (2H, m), 10.31-10.23 (1H, m), 13.28-13.27 (1H, m). |
| 6.15 | (CD2Cl2) 1.50-1.44 (3H, m), 1.85-1.77 (1H, m), 2.17-2.04 (2H, m), 2.25-2.19 |
| | (1H, m), 2.70-2.62 (2H, m), 3.45-3.39 (1H, m), 4.59-4.45 (2H, m), 4.70-4.62 (1H, |
| | m), 6.96-6.89 (1H, m), 7.01-6.97 (1H, m), 7.10-7.05 (2H, m), 7.45-7.39 (1H, m), |
| | 7.62-7.58 (1H, m), 7.76-7.66 (2H, m), 7.88-7.83 (1H, m), 8.50-8.41 (2H, m) |
| 6.16 | 1.31-1.28 (3H, m), 1.99-1.92 (2H, m), 3.59-3.44 (3H, m), 4.47-4.38 (5H, m), |
| | 6.74-6.71 (2H, m), 6.84 (1H, d, J = 5.6 Hz), 7.35-7.29 (6H, m), 7.50 (1H, d, J = 0.8 |
| | Hz), 7.78 (1H, d, J = 5.8 Hz), 8.22-8.12 (2H, m), 8.48 (1H, t, J = 6.0 Hz). |
| 6.17 | 1.33-1.24 (3H, m), 3.64 (1H, t, J = 5.4 Hz), 4.11-4.00 (2H, m), 4.46-4.37 (3H, m), |
| | 6.73 (2H, s), 6.95-6.84 (4H, m), 7.36-7.24 (3H, m), 7.54-7.49 (1H, m), 7.78 (1H, |
| | d, J = 5.8 Hz), 8.16-8.12 (1H, m), 8.37-8.28 (1H, m), 8.53 (1H, t, J = 6.0 Hz). |
| 6.18 | 1.29 (3H, d, J = 7.2 Hz), 1.71-1.48 (4H, m), 2.60-2.54 (2H, m), 3.52-3.45 (1H, m), |
| | 4.44-4.21 (3H, m), 6.78 (2H, s), 6.86-6.84 (1H, m), 7.20-7.17 (3H, m), 7.37-7.26 |
| | (3H, m), 7.51 (1H, s), 7.78 (1H, d, J = 5.8 Hz),, 8.13 (1H, d, J = 8.8 Hz), 8.25 (2H, s), |
| | 8.45 (1H, d, J = 7.3 Hz), 8.64 (1H, t, J = 6.0 Hz). |
| 6.19 | 1.16-0.97 (5H, m), 1.29 (3H, d, J = 7.0 Hz), 1.69-1.47 (5H, m), 1.85-1.71 (2H, m), |
| | 1.96-1.90 (1H, m), 2.28-2.22 (1H, m), 2.62-2.55 (1H, m), 2.72-2.64 (1H, m), |
| | 3.15-3.08 (1H, m), 4.46-4.37 (3H, m), 6.71 (2H, s), 6.82 (1H, d, J = 5.6 Hz), 7.19- |
| | 7.16 (3H, m), 7.36-7.23 (3H, m), 7.52-7.50 (1H, m), 7.77-7.75 (1H, m), 8.19- |
| | 8.11 (2H, m), 8.56 (1H, t, J = 6.0 Hz). |
| 6.20 | 1.33-1.26 (3H, m), 1.97-1.78 (2H, m), 2.63-2.55 (1H, m), 2.76-2.66 (1H, m), |
| | 2.80 (3H, s), 3.99-3.91 (1H, m), 4.56-4.31 (3H, m), 7.06 (1H, d, J = 6.5 Hz), 7.22- |
| | 7.18 (3H, m), 7.31-7.27 (2H, m), 7.56-7.50 (2H, m), 7.73-7.70 (2H, m), 8.16- |
| | 8.13 (2H, m), 8.44-8.34 (2H, m), 8.66-8.61 (1H, m). |
| 6.21 | 1.33-1.29 (3H, m), 1.75-1.63 (1H, m), 1.96-1.85 (1H, m), 2.71-2.55 (2H, m), |
| | 3.29-3.26 (1H, m), 4.56-4.29 (3H, m), 6.81-6.75 (2H, m), 6.88 (1H, dd, J = 3.1, 5.6 |
| | Hz), 7.27-7.12 (5H, m), 7.51-7.47 (1H, m), 7.63 (1H, dd, J = 5.4, 8.3 Hz), 7.77 (1H, |
| | dd, J = 1.6, 5.7 Hz), 8.04 (1H, s), 8.30-8.24 (1H, m), 8.58-8.50 (1H, m). |
| 6.22 | 1.36 (3H, d, J = 7.0 Hz), 2.09-1.99 (2H, m), 2.69-2.61 (2H, m), 4.01-3.92 (4H, m), |
| | 4.52-4.37 (3H, m), 6.58-6.50 (2H, m), 7.26-7.17 (4H, m), 7.33 (2H, t, J = 7.3 Hz), |
| | 7.60 (1H, d, J = 6.9 Hz), 7.69 (1H, s), 7.99 (1H, s), 8.25 (3H, d, J = 4.4 Hz), 8.67 (1H, t, |
| | J = 6.0 Hz), 8.95-8.82 (2H, m). |
| 6.23 | 1.38 (3H, d, J = 7.0 Hz), 1.83-1.85 (2H, m), 1.91-1.94 (2H, m), 2.04-2.09 (1H, m), |
| | 2.18-2.21 (1H, m), 2.54-2.60 (2H, m), 2.78 (1H, s), 3.06-3.07 (1H, m), 3.17-3.21 |
| | (1H, m), 3.52-3.57 (1H, m), 3.87-3.91 (1H, m), 4.42 (1H, t, J = 6.9 Hz), 4.52 (2H, d, |
| | J = 6.7 Hz), 7.18-7.24 (4H, m), 7.30-7.34 (2H, m), 7.64-7.69 (2H, m), 7.77 (1H, s), |
| | 8.49 (1H, d, J = 8.7 Hz), 8.84 (1H, t, J = 5.9 Hz), 8.97-9.01 (2H, m), 9.08 (1H, d, J = 6.7 |
| | Hz), 10.09 (1H, br, s), 13.08 (1H, br, s) |
| 6.24 | 1.30 (3H, d, J = 7.0 Hz), 1.81-1.69 (1H, m), 1.99-1.88 (1H, m), 2.64 (2H, t, J = 8.1 |
| | Hz), 3.90 (1H, td, J = 4.1, 8.3 Hz), 4.35 (1H, quin, J = 7.2 Hz), 4.43 (2H, d, J = 5.9 Hz), |
| | 5.70 (1H, d, J = 5.2 Hz), 6.71 (2H, s), 6.80 (1H, d, J = 5.9 Hz), 7.18-7.13 (3H, m), |
| | 7.27-7.22 (2H, m), 7.32 (1H, dd, J = 1.4, 8.6 Hz), 7.50 (1H, s), 7.74 (1H, d, J = 5.8 |
| | Hz), 7.81 (1H, d, J = 7.6 Hz), 8.12 (1H, d, J = 8.6 Hz), 8.57 (1H, t, J = 5.9 Hz). |
| 6.25 | 1.27 (3H, d, J = 7.1 Hz), 1.33 (9H, s), 1.92-1.75 (2H, m), 2.57-2.51 (1H, m), 2.69- |
| | 2.58 (1H, m), 3.96-3.90 (1H, m), 4.33 (1H, quin, J = 7.2 Hz), 4.38 (1H, dd, J = 6.0, |
| | 16.0 Hz), 4.45 (1H, dd, J = 6.1, 15.7 Hz), 6.71 (2H, s), 6.82 (1H, d, J = 5.8 Hz), 7.22- |
| | 7.13 (4H, m), 7.30-7.24 (2H, m), 7.32 (1H, dd, J = 1.6, 8.6 Hz), 7.49 (1H, s), 7.75 |
| | (1H, d, J = 5.8 Hz), 8.11 (1H, d, J = 8.6 Hz), 8.17 (1H, d, J = 7.5 Hz), 8.40 (1H, t, J = 5.9 |
| | Hz). |
| 6.26 | 1.30 (3H, d, J = 7.2 Hz), 2.05 (2H, q, J = 7.8 Hz), 2.62 (1H, td, J = 8.2, 13.8 Hz), 2.73 |
| | (1H, td, J = 7.7, 14.0 Hz), 4.38-4.30 (1H, m), 4.50-4.38 (3H, m), 6.67 (2H, s), 6.77 |
| | (1H, d, J = 5.8 Hz), 7.24-7.16 (3H, m), 7.31-7.26 (2H, m), 7.33 (1H, dd, J = 1.6, 8.7 |
| | Hz), 7.38 (2H, t, J = 7.6 Hz), 7.52-7.48 (2H, m), 7.71 (1H, d, J = 5.8 Hz), 7.78-7.74 |
| | (2H, m), 8.09 (1H, d, J = 8.6 Hz), 8.42 (1H, d, J = 7.6 Hz), 8.47 (1H, t, J = 6.1 Hz), 8.68 |
| | (1H, d, J = 7.0 Hz) |
| 9.01 | 1.24 (3H, d, J = 7.0 Hz), 1.66-1.55 (1H, m), 1.96-1.81 (3H, m), 2.68-2.52 (2H, m), |
| | 3.16 (1H, dd, J = 4.8, 8.1 Hz), 4.33-4.25 (1H, m), 4.42-4.39 (2H, m), 7.18-7.14 |
| | (3H, m), 7.27-7.23 (2H, m), 7.65 (1H, s), 7.79 (1H, d, J = 2.0 Hz), 8.12 (1H, d, J = 6.6 |
| | Hz), 8.22 (1H, d, J = 2.0 Hz), 8.51-8.46 (1H, m), 11.93 (1H, s). |
| 9.02 | 8.29-8.24 (1H, m), 8.14 (1H, d, J = 7.0 Hz), 7.77-7.71 (1H, m), 7.31-7.17 (5H, m), |
| | 6.42-6.34 (1H, m), 5.80-5.76 (2H, m), 5.20 (1H, q, J = 8.3 Hz), 4.34-4.27 (1H, m), |
| | 3.15-3.22 (1H, m), 2.61-2.80 (2H, m), 2.35-2.48 (1H, m), 1.94-1.84 (1H, m), |
| | 1.80-1.60 (2H, m), 1.26 (3H, t, J = 7.0 Hz). |
| 9.03 | 0.92 (6H, t, J = 5.6 Hz), 1.24 (3H, d, J = 6.9 Hz), 1.82-1.60 (2H, m), 2.56-2.67 (2H, |
| | m). 3.06 (1H, dd, J = 5.6, 7.6 Hz), 3.31-3.33 (2H, m), 4.45-4.34 (3H, m), 7.19-7.15 |
| | (3H, m), 7.29-7.23 (2H, m), 7.68-7.66 (1H, m), 7.81 (1H, d, J = 1.9 Hz), 8.14 (1H, d, |
| | J = 7.8 Hz), 8.24 (1H, d, J = 1.9 Hz), 8.48 (1H, t, J = 5.9 Hz), 11.96-11.91 (1H, m). |
| 9.04 | 1.26-1.23 (3H, m), 1.67-1.56 (1H, m), 1.93-1.83 (1H, m), 2.08 (2H, s), 2.70-2.53 |
| | (2H, m), 3.20-3.15 (1H, m), 4.39-4.30 (3H, m), 7.08 (1H, dd, J = 1.6, 7.9 Hz), 7.19- |
| | 7.13 (3H, m), 7.28-7.23 (2H, m), 7.36-7.33 (2H, m), 7.48 (1H, d, J = 2.6 Hz), 8.12 |
| | (1H, d, J = 6.9 Hz), 8.43 (1H, t, J = 5.9 Hz), 11.30-11.28 (1H, m). |
| 9.05 | 1.12-1.09 (3H, m), 1.65-1.55 (1H, m), 1.92-1.79 (3H, m), 2.66-2.54 (2H, m), |
| | 3.13 (1H, dd, J = 4.6, 7.8 Hz), 3.93 (3H, s), 4.24-4.09 (3H, m), 6.92 (1H, d, J = 2.1 |
| | Hz), 7.20-7.18 (3H, m), 7.31-7.25 (4H, m), 8.04-8.01 (1H, m), 8.23-8.15 (2H, m). |
| 9.06 | 1.33-1.30 (3H, m), 2.06-1.98 (2H, m), 2.56 (3H, s), 2.70-2.57 (2H, m), 3.95-3.90 |
| | (1H, m), 4.55-4.44 (3H, m), 7.26-7.18 (3H, m), 7.40-7.30 (3H, m), 7.64-7.60 (1H, |
| | m), 7.73 (1H, d, J = 7.3 Hz), 8.39-8.22 (4H, m), 8.70 (1H, t, J = 5.8 Hz), 8.83-8.80 |
| | (1H, m), 8.95-8.88 (1H, m). |
| 9.07 | 1.28-1.25 (3H, m), 1.69-1.58 (1H, m), 1.94-1.83 (1H, m), 2.09-2.03 (2H, m), |
| | 2.79-2.55 (2H, m), 3.22-3.15 (1H, m), 4.33 (1H, t, J = 6.1 Hz), 4.53-4.47 (2H, m), |
| | 6.45 (2H, s), 7.01 (1H, d, J = 6.0 Hz), 7.21-7.17 (3H, m), 7.30-7.25 (2H, m), 7.46 |
| | (1H, s), 7.71 (1H, d, J = 5.6 Hz), 8.19-8.14 (1H, m), 8.66 (1H, t, J = 5.9 Hz). |
| 9.08 | 0.93-0.95 (6H, m), 1.24 (4H, d, J = 6.9 Hz), 1.64-1.70 (1H, m), 1.74-1.78 (1H, m), |
| | 2.56-2.68 (3H, m), 3.08-3.12 (1H, m), 4.32-4.39 (3H, m), 7.07 (1H, dd, J = 1.4, 8.4 |
| | Hz), 7.14-7.18 (3H, m), 7.24-7.28 (2H, m), 7.33-7.38 (2H, m), 7.49 (1H, d, J = 2.7 |
| | Hz), 8.16-8.21 (1H, m), 8.43 (1H, t, J = 5.8 Hz), 11.31 (1H, s) |
| 9.09 | 0.95-0.97 (6H, m), 1.26 (3H, d, J = 7.0 Hz), 1.66-1.71 (1H, m), 1.75-1.82 (1H, m), |
| | 2.54-2.71 (3H, m), 3.10 (2H, t, J = 5.6 Hz), 4.05-4.19 (2H, m), 4.33-4.40 (1H, m), |
| | 5.84 (2H, s), 6.27 (1H, s), 6.33 (1H, dd, J = 5.2, 1.2 Hz), 7.15-7.19 (3H, m), 7.25- |
| | 7.29 (2H, m), 7.77 (1H, d, J = 5.2 Hz), 8.17 (1H, s), 8.45 (1H, t, J = 6.0 Hz) |
| 9.10 | 1.23-1.27 (6H, m), 1.31 (3H, d, J = 7.1 Hz), 2.00-2.11 (2H, m), 2.57-2.68 (2H, m), |
| | 3.15-3.24 (1H, m), 3.99 (3H, s), 4.31 (2H, d, J = 5.9 Hz), 4.39 (1H, t, J = 7.2 Hz), 7.19- |
| | 7.24 (3H, m), 7.28-7.33 (4H, m), 7.41 (2H, d, J = 8.1 Hz), 8.32 (3H, s), 8.72 (1H, t, J = |
| | 5.9 Hz), 8.96 (1H, s), 9.08 (1H, d, J = 7.1 Hz), 9.13 (1H, s) |
| 9.11 | 0.94-0.96 (6H, m), 1.23 (3H, d, J = 7.0 Hz), 1.61-1.82 (2H, m), 2.54-2.68 (3H, m), |
| | 3.09-3.12 (2H, m), 3.80 (3H, s), 4.21 (2H, t, J = 5.4 Hz), 4.33 (1H, t, J = 7.4 Hz), 7.05 |
| | (1H, d, J = 8.5 Hz), 7.14-7.18 (4H, m), 7.24-7.29 (3H, m), 8.17-8.19 (2H, m), 8.43 |
| | (1H, t, J = 5.9 Hz) |
| 9.12 | 0.99 (6H, t, J = 6.3 Hz), 1.18 (3H, d, J = 6.9 Hz), 1.62-1.86 (2H, m), 2.50-2.56 (2H, |
| | m), 2.61-2.78 (2H, m), 3.09-3.39 (5H, m), 4.20-4.32 (1H, m), 5.76 (2H, s), 6.25 |
| | (1H, s), 6.33 (1H, dd, J = 5.2, 1.5 Hz), 7.15-7.22 (3H, m), 7.24-7.31 (2H, m), 7.77 |
| | (1H, d, J = 5.2 Hz), 8.05 (1H, t, J = 5.7 Hz), 8.17 (1H, s) |
| 9.16 | 1.26 (3H, d, J = 7.1 Hz), 1.74-1.93 (2H, m), 2.45-2.57 (2H, m), 2.80 (3H), 3.88- |
| | 3.94 (1H, m), 4.28-4.39 (3H, m), 7.07 (1H, dd, J = 8.5, 1.5 Hz), 7.16-7.19 (2H, m), |
| | 7.26-7.29 (2H, m), 7.33-7.35 (2H, m), 7.48-7.52 (2H, m), 8.33 (1H, d, J = 7.4 Hz), |
| | 8.43 (1H, t, J = 5.9 Hz), 8.55 (1H, s), 11.31 (1H, s) |
| 9.17 | 1.26 (3H, d, J = 5.6 Hz), 1.60-1.91 (4H, m), 2.54-2.65 (3H, m), 3.15-3.22 (3H, |
| | m), 3.75-3.82 (3H, m), 4.38 (4H, d, J = 5.8 Hz), 7.07 (1H, dd, J = 8.4, 1.0 Hz), 7.17 |
| | (4H, d, J = 7.4 Hz), 7.24-7.28 (3H, m), 7.31-7.36 (3H, m), 7.49 (1H, d, J = 2.5 Hz), |
| | 8.14 (1H, br.s), 11.31 (1H, br.s) |
| 9.18 | 0.85 (9H, s), 1.25 (3H, d, J = 7.0 Hz), 1.68-1.88 (2H, m), 2.06-2.23 (2H, m), 2.55- |
| | 2.70 (2H, m), 2.90-2.97 (1H, m), 4.34-4.44 (3H, m), 7.08 (1H, dd, J = 8.4, J = 1.5 |
| | Hz), 7.14-7.20 (3H, m), 7.23-7.29 (2H, m), 7.32-7.37 (2H, m), 7.49 (1H, d, J = 2.6 |
| | Hz), 8.04 (1H, d, J = 8.00 Hz), 8.44 (1H, t, J = 8.4 Hz), 11.32 (1H, s). |
| 9.19 | 0.89-0.92 (6H, m), 1.06 (3H, d, J = 7.0 Hz), 1.32 (3H, t, J = 7.1 Hz), 1.56-1.64 (1H, |
| | m), 1.68-1.78 (1H, m), 2.52-2.58 (2H, m), 2.59-2.63 (1H, m), 2.99-3.02 (1H, m), |
| | 3.92 (3H, s), 4.05-4.11 (1H, m), 4.17-4.23 (2H, m), 4.34 (2H, q, J = 7.1 Hz), 7.14- |
| | 7.17 (3H, m), 7.24-7.27 (2H, m), 7.30-7.38 (2H, m), 8.01 (1H, d, J = 7.9 Hz), 8.18 |
| | (1H, t, J = 4.9 Hz), 8.28 (1H, s), 9.06 (1H, s) |
| 9.20 | 0.90-0.95 (6H, m), 1.21 (3H, d, J = 7.0 Hz), 1.63-1.67 (1H, m), 1.73-1.77 (1H, m), |
| | 2.53-2.68 (3H, m), 2.93-3.00 (1H, m), 3.02-3.07 (1H, m), 3.26-3.29 (3H, m), |
| | 4.00-4.02 (1H, m), 4.30-4.34 (1H, m), 5.95 (1H, s), 6.56 (1H, d, J = 8.3 Hz), 6.67- |
| | 6.71 (2H, m), 7.14-7.18 (3H, m), 7.24-7.28 (2H, m), 8.11-8.16 (2H, m), 8.30 (1H, |
| | s) |
| 9.21 | 1.25 (3H, d, J = 7.1 Hz), 1.77-1.89 (2H, m), 2.52-2.71 (2H, m), 2.78 (3H, s), 3.87- |
| | 3.93 (1H, m), 4.28 (1H, t, J = 7.2 Hz), 4.33-4.46 (2H, m), 7.15-7.19 (3H, m), 7.25- |
| | 7.30 (2H, m), 7.49 (1H, d, J = 8.0 Hz), 7.66 (1H, s), 7.78 (1H, d, J = 2.0 Hz), 8.21 (1H, |
| | d, J = 2.0 Hz), 8.35 (1H, d, J = 7.3 Hz), 8.50 (1H, t, J = 5.8 Hz), 11.92 (1H, s) |
| 9.22 | 1.17-1.30 (7H, m), 1.55-1.58 (1H, m), 1.64-1.68 (1H, m), 1.73-1.76 (2H, m), |
| | 3.11-3.21 (4H, m), 3.72-3.80 (2H, m), 4.32-4.45 (3H, m), 7.11-7.30 (6H, m), |
| | 7.66 (1H, d, J = 2.6 Hz), 7.79 (1H, br.s), 8.15 (1H, dd, J = 20.0, 7.9 Hz), 8.21 (1H, t, J = |
| | 1.8 Hz), 8.43 (1H, br.s), 8.48-8.59 (1H, m), 11.93 (1H, s) |
| 9.23 | 0.90-0.94 (6H, m), 1.25 (3H, d, J = 7.0 Hz), 1.62-1.68 (1H, m), 1.71-1.83 (1H, m), |
| | 2.54-2.67 (3H, m), 3.05-3.08 (1H, m), 4.34-4.41 (1H, m), 4.52-4.64 (2H, m), |
| | 7.13-7.17 (3H, m), 7.23-7.33 (3H, m), 7.35 (1H, s), 7.43 (1H, d, J = 7.2 Hz), 7.85 |
| | (1H, d, J = 8.0 Hz), 8.16 (1H, d, J = 7.9 Hz), 8.34 (1H, s), 8.73 (1H, t, J = 5.9 Hz) |
| 9.24 | 0.94-0.97 (6H, m), 1.25 (3H, d, J = 7.0 Hz), 1.63-1.73 (1H, m), 1.76-1.84 (1H, m), |
| | 2.54-2.68 (3H, m), 3.15 (1H, t, J = 6.8 Hz), 4.33-4.42 (3H, m), 6.43 (2H, s), 6.71 |
| | (1H, d, J = 5.5 Hz), 6.79 (1H, s), 7.15-7.18 (3H, m), 7.25-7.28 (2H, m), 7.72 (1H, d, |
| | J = 5.9 Hz), 8.18 (2H, s), 8.24 (1H, d, J = 7.9 Hz), 8.58 (1H, t, J = 5.6 Hz) |
| 9.25 | 1.25 (3H, d, J = 7.0 Hz), 1.67-1.85 (2H, m), 2.53-2.60 (1H, m), 2.61-2.70 (1H, m), |
| | 3.00-3.05 (1H, m), 3.53 (1H, d, J = 13.4 Hz), 3.66 (1H, d, J = 13.4 Hz), 4.35-4.43 |
| | (3H, m), 7.08 (1H, dd, J = 8.4, 1.5 Hz), 7.11-7.17 (3H, m), 7.19-7.26 (3H, m), 7.28- |
| | 7.31 (4H, m), 7.33 (1H, d, J = 8.4 Hz), 7.37 (1H, s), 7.48 (1H, d, J = 2.6 Hz), 8.15 (1H, |
| | d, J = 7.8 Hz), 8.30 (1H, s), 8.44 (1H, t, J = 5.9 Hz), 11.32 (1H, s) |
| 9.26 | 0.83 (9H, s), 1.23 (3H, d, J = 7.0 Hz), 1.68-1.86 (2H, m), 2.12 (2H, s), 2.56-2.67 |
| | (2H, m), 2.88-2.94 (1H, m), 4.29-4.47 (3H, m), 7.12-7.21 (3H, m), 7.22-7.29 (2H, |
| | m), 7.65 (1H, d, J = 2.7 Hz), 7.79 (1H, d, J = 1.6 Hz), 8.04 (1H, d, J = 7.9 Hz), 8.22 (1H, |
| | d, J = 2.0 Hz), 8.33 (1H, s), 8.50 (1H, t, J = 5.9 Hz), 11.93 (1H, s) |
| 9.27 | 0.92-0.96 (6H, m), 1.24 (3H, d, J = 7.0 Hz), 1.61-1.70 (1H, m), 1.72-1.82 (1H, m), |
| | 2.15 (1H, s), 2.54-2.67 (3H, m), 3.09-3.13 (1H, m), 4.31-4.39 (1H, m), 4.44-4.55 |
| | (2H, m), 6.46 (2H, s), 6.98 (1H, d, J = 5.7 Hz), 7.14-7.18 (3H, m), 7.24-7.28 (2H, |
| | m), 7.45 (1H, s), 7.70 (1H, d, J = 5.6 Hz), 8.19 (2H, s), 8.21 (1H, s), 8.68 (1H, t, J = 5.8 |
| | Hz) |
| 10.01 | 0.92-0.81 (6H, m), 1.49-1.20 (12H, m), 2.31-2.00 (6H, m), 2.52-2.49 (3H, m), |
| | 2.93-2.88 (1H, m), 3.09 (1H, t, J = 5.8 Hz), 3.86-3.80 (1H, m), 4.21-4.04 (1H, m), |
| | 4.45-4.38 (2H, m), 4.73-4.68, 4.90-4.85 (1H, m), 7.13-7.06 (1H, m), 7.37 (2H, |
| | dd, J = 8.3, 12.9 Hz), 7.51-7.48 (1H, m), 8.45-8.40, 8.80-8.75 (1H, m), 11.32 (1H, |
| | d, J = 6.3 Hz). |
| 10.03 | 1.15-1.59 (8H, m), 2.33-2.00 (8H, m), 2.92-2.86 (0.6H, m), 3.18 (0.4H, t, J = 6.5 |
| | Hz), 3.58-3.48 (4H, m), 3.81 (1H, t, J = 7.6 Hz), 4.10-4.03 (0.6H, m), 4.22-4.15 |
| | (0.4H, m), 4.47-4.41 (2H, m), 4.61 (0.6H, dd, J = 5.6, 9.0 Hz), 4.94-4.88 (0.4H, m), |
| | 7.67 (1H, d, J = 3.7 Hz), 7.84-7.82 (1H, m), 8.26-8.23 (1H, m), 8.59-8.53 (0.6H, |
| | m), 8.81-8.78 (0.4H, m), 11.93 (1H, s). |
| 10.04 | 1.70-0.94 (4H, m), 2.29-2.00 (6H, m), 2.85-2.35 (8H, m), 2.89 (0.5H, t, J = 6.5 |
| | Hz), 3.17 (0.5H, t, J = 6.1 Hz), 3.85-3.77 (1H, m), 4.11-4.03 (0.5H, m), 4.23-4.15 |
| | (0.5H, m), 4.47-4.40 (2H, m), 4.61 (0.5H, dd, J = 5.6, 9.1 Hz), 4.91 (0.5H, dd, J = 5.3, |
| | 9.0 Hz), 7.67-7.65 (1H, m), 7.83 (1H, s), 8.27-8.23 (1H, m), 8.57 (0.5H, t, J = 5.8 |
| | Hz), 8.80 (0.5H, t, J = 5.7 Hz), 11.94-11.88 (1H, m). |
| 10.05 | 0.94-0.83 (6H, m), 1.14-1.00 (2H, m), 1.40-1.25 (4H, m), 2.59-1.98 (10H, m), |
| | 2.88-2.85 (0.5H, m), 3.10-3.07 (0.5H, m), 3.57-3.47 (4H, m), 4.23-3.80 (2H, m), |
| | 4.50-4.37 (2H, m), 4.69 (0.5H, dd, J = 5.5, 9.0 Hz), 4.85 (0.5H, dd, J = 5.3, 8.9 Hz), |
| | 7.67 (1H, d, J = 6.0 Hz), 7.82 (1H, t, J = 2.2 Hz), 8.27-8.22 (1H, m), 8.55-8.49 (0.5H, |
| | m), 8.86 (0.5H, t, J = 5.7 Hz), 11.93 (1H, s) |
| 10.06 | 1.46-1.14 (6H, m), 1.70-1.58 (2H, m), 2.19-2.03 (1H, m), 2.49-2.44 (1H, m), |
| | 2.97-2.91 (0.3H, m), 3.19-3.13 (0.7H, m), 3.42-3.28 (1H, m), 3.84-3.77 (1H, m), |
| | 4.10-4.02 (1H, m), 4.23-4.15 (1H, m), 4.46-4.32 (3H, m), 4.66-4.60 (0.7H, m), |
| | 4.95-4.89 (0.3H, m), 7.13-7.08 (1H, m), 7.40-7.34 (2H, m), 7.50-7.48 (1H, m), |
| | 8.49 (0.7H, t, J = 5.7 Hz), 8.70 (0.3H, t, J = 5.6 Hz), 11.32 (1H, s). |
| 10.07 | 0.95-0.94 (6H, d, J = 6.0 Hz), 1.58-0.97 (8H, m), 2.34-2.06 (2H, m), 3.08-2.62 |
| | (4H, m), 3.63-3.24 (5H, m), 4.32-3.96 (5H, m), 4.40 (1H, t, J = 8.6 Hz), 4.57-4.52 |
| | (1H, m), 4.92 (0.5H, d, J = 9.3 Hz), 5.23-5.20 (0.5H, m), 6.52 (0.5H, dd, J = 0.9, 8.5 |
| | Hz), 6.61 (0.5H, dd, J = 1.2, 8.4 Hz), 7.35-7.09 (5H, m), 7.48 (1H, dd, J = 2.6, 5.3 |
| | Hz), 8.20-8.19 (0.5H, m), 8.52-8.49 (0.5H, m), 11.30-11.26 (1H, m). |
| 10.08 | 1.49-0.98 (6H, m), 1.69-1.66 (1H, m), 2.19-2.04 (2H, m), 2.47-2.27 (7H, m), |
| | 2.97-2.91 (0.5H, m), 3.21-3.17 (0.5H, m), 3.73-3.64 (4H, m), 3.81 (1H, t, J = 7.6 |
| | Hz), 4.12-4.04 (0.5H, m), 4.24-4.16 (0.5H, m), 4.40 (2H, ddd, J = 5.9, 14.7, 23.1 |
| | Hz), 4.64 (0.5H, dd, J = 5.6, 9.1 Hz), 4.93 (0.5H, dd, J = 5.3, 9.0 Hz), 6.62-6.58 (1H, |
| | m), 7.13-7.08 (1H, m), 7.40-7.35 (2H, m), 7.50-7.48 (1H, m), 8.34 (2H, dd, J = |
| | 1.8, 4.7 Hz), 8.49 (0.5H, t, J = 5.8 Hz), 8.72 (0.5H, t, J = 5.7 Hz), 11.31 (1H, s). |
| 10.09 | 1.56-1.06 (6H, m), 1.76-1.65 (1H, m), 2.17-2.05 (1H, m), 2.25 (1H, t, J = 7.2 Hz), |
| | 2.65-2.38 (6H, m), 2.82-2.73 (2H, m), 2.98-2.93 (0.5H, m), 3.21-3.17 (0.5H, m), |
| | 3.44 (1H, s), 3.52-3.49 (1H, m), 3.81 (1H, t, J = 7.6 Hz), 4.11-4.04 (0.5H, m), 4.22- |
| | 4.15 (0.5H, m), 4.46-4.32 (2H, m), 4.65-4.60 (0.5H, m), 4.96-4.90 (0.5H, m), 7.12- |
| | 7.00 (4H, m), 7.39-7.33 (2H, m), 7.49 (1H, d, J = 2.6 Hz), 8.48 (0.5H, t, J = 5.8 Hz), |
| | 8.71 (0.5H, t, J = 5.7 Hz), 11.31 (1H, s). |
| 10.10 | 1.51-1.09 (6H, m), 2.18-1.59 (4H, m), 2.68-2.48 (3H, m), 2.97-2.93 (0.5H, m), |
| | 3.20-3.18 (0.5H, m), 3.75-3.73 (2H, m), 3.83-3.79 (3H, m), 4.08 (0.5H, dd, J = |
| | 8.4, 14.3 Hz), 4.23-4.16 (0.5H, dd, J = 8.4, 14.3 Hz), 4.47-4.33 (2H, m), 4.66-4.60 |
| | (0.5H, m), 4.96-4.90 (0.5H, m), 7.24-7.07 (4H, m), 7.39-7.33 (2H, m), 7.49 (1H, d, |
| | J = 2.4 Hz), 8.48 (0.5H, t, J = 5.8 Hz), 8.72 (0.5H, t, J = 5.7 Hz), 11.31 (1H, s). |
| 10.11 | 0.93-0.82 (6H, m), 1.37-1.12 (4H, m), 1.58-1.43 (3H, m), 2.25-2.05 (1H, m), |
| | 2.65-2.38 (5H, m), 2.93 (0.5H, s), 3.15-3.11 (0.5H, m), 3.86-3.71 (4.5H, m), 4.21- |
| | 4.07 (1.5H, m), 4.49-4.33 (2H, m), 4.71 (0.5H, dd, J = 5.6, 9.0 Hz), 4.88 (0.5H, dd, |
| | J = 5.3, 8.9 Hz), 7.24-7.07 (4H, m), 7.38-7.33 (2H, m), 7.49 (1H, s), 8.43 (0.5H, t, J = |
| | 5.7 Hz), 8.79 (0.5H, t, J = 5.6 Hz), 11.34-11.29 (1H, m). |
| 10.12 | 1.50-0.97 (7H, m), 1.98-1.82 (5H, m), 2.16-2.06 (2H, m), 2.47-2.27 (6H, m), |
| | 2.96-2.90 (0.5H, m), 3.19-3.15 (0.5H, m), 3.80 (1H, t, J = 7.6 Hz), 4.10-4.03 |
| | (0.5H, m), 4.23-4.16 (0.5H, m), 4.47-4.35 (2H, m), 4.63 (0.5H, dd, J = 5.6, 9.1 Hz), |
| | 4.95-4.90 (0.5H, m), 7.13-7.08 (1H, m), 7.40-7.34 (2H, m), 7.50-7.48 (1H, m), |
| | 8.48 (0.5H, t, J = 5.8 Hz), 8.71 (0.5H, t, J = 5.8 Hz), 11.31-11.31 (1H, m). |
| 10.13 | 1.49-0.95 (6H, m), 1.73-1.60 (3H, m), 2.16-2.06 (1H, m), 2.93-2.88 (0.5H, m), |
| | 3.18-3.12 (0.5H, m), 3.84-3.69 (2H, m), 3.93 (1H, t, J = 7.0 Hz), 4.07-4.00 (0.5H, |
| | m), 4.22-4.14 (0.5H, m), 4.47-4.26 (2H, m), 4.61 (0.5H, dd, J = 5.6, 9.1 Hz), 4.93 |
| | (0.5H, dd, J = 5.3, 9.1 Hz), 6.86 (1H, d, J = 10.0 Hz), 7.10 (2H, dd, J = 13.3, 21.7 Hz), |
| | 7.41-7.34 (2H, m), 7.53-7.49 (1.5H, m), 7.60 (0.5H, s), 8.48 (0.5H, t, J = 5.8 Hz), |
| | 8.73 (0.5H, t, J = 5.8 Hz), 11.36-11.29 (1H, m). |
| 10.14 | 1.60-1.11 (6H, m), 2.18-2.04 (1H, m), 2.47-2.37 (1H, m), 2.90-2.78 (3H, m), |
| | 3.03-2.93 (1.5H, m), 3.30-3.17 (4.5H, m), 3.85-3.78 (1H, m), 4.10-4.02 (0.5H, |
| | m), 4.23-4.16 (0.5H, m), 4.47-4.31 (2H, m), 4.62 (0.5H, dd, J = 5.6, 9.1 Hz), 4.93 |
| | (0.5H, dd, J = 5.3, 9.1 Hz), 6.37 (0.5H, d, J = 7.7 Hz), 6.56-6.44 (1.5H, m), 7.02- |
| | 6.91 (2H, m), 7.10 (1H, dd, J = 2.8, 8.3 Hz), 7.39-7.34 (2H, m), 7.50-7.48 (1H, m), |
| | 8.49 (0.5H, t, J = 5.8 Hz), 8.75-8.70 (0.5H, m), 11.31 (1H, s). |
| 10.15 | 1.33-1.01 (4H, m), 1.50-1.35 (3H, m), 1.65-1.58 (3H, m), 1.90-1.77 (2H, m), |
| | 2.19-2.05 (2H, m), 2.38-2.27 (3H, m), 2.60-2.40 (3H, m), 2.93 (0.5H, dd, J = 5.0, |
| | 7.3 Hz), 3.18-3.14 (0.5H, m), 3.84-3.77 (1H, m), 4.10-4.03 (0.5H, m), 4.22-4.16 |
| | (0.5H, m), 4.47-4.32 (2H, m), 4.63 (0.5H, dd, J = 5.6, 9.0 Hz), 4.95-4.89 (0.5H, m), |
| | 7.13-7.08 (1H, m), 7.40-7.34 (2H, m), 7.50-7.48 (1H, m), 8.48 (0.5H, t, J = 5.8 |
| | Hz), 8.71 (0.5H, t, J = 5.6 Hz), 11.32 (1H, d, J = 0.7 Hz). |
| 10.16 | 1.05-1.22 (13H, m), 2.25-2.02 (9H, m), 3.82-3.74 (1H, m), 4.08-4.01 (1H, m), |
| | 4.44-4.37 (2H, m), 4.65-4.61 (0.5H, m), 4.81-4.76 (0.5H, m), 7.12-7.07 (1H, m), |
| | 7.39-7.34 (2H, m), 7.50-7.48 (1H, m), 8.45 (0.5H, m), 8.76 (0.5H, m,), 11.31 (1H, |
| | s). |
| 10.17 | 0.96-0.86 (6H, m), 1.93-1.84 (1H, m), 2.26-2.06 (1H, m), 2.54-2.36 (2H, m), |
| | 2.76-2.65 (1H, m), 3.64-3.42 (1H, m), 3.93-3.83 (2H, m), 4.12-3.96 (1H, m), |
| | 4.22 (1H, t, J = 7.6 Hz), 4.43-4.35 (2H, m), 4.72 (0.7H, dd, J = 6.8, 9.3 Hz), 5.02- |
| | 4.99 (0.3H, m), 6.97-6.75 (2H, m), 7.11-7.07 (1H, m), 7.38-7.19 (4H, m), 7.50- |
| | 7.48 (1H, m), 8.42-8.39 (0.7H, m), 8.76-8.72 (0.3H, m), 11.33-11.28 (1H, m). |
| 10.18 | 0.84-0.87 (6H, m), 1.48-1.78 (1H, m), 1.82-2.03 (1H, m), 2.57-2.58 (1H, m), |
| | 2.73 (1H, s), 2.84-2.94 (1H, m), 3.82-3.86 (2H, m), 4.36-4.40 (4H, m), 6.98-7.09 |
| | (3H, m), 7.19 (1H, d, J = 2.2 Hz), 7.33-7.36 (3H, m), 7.49 (2H, m), 8.17 (1H, s), 8.34 |
| | (1H, t, J = 5.8 Hz), 10.90 (1H, s), 11.32 (1H, s) |
| 10.19 | 1.17-1.31 (2H, m), 1.33-1.51 (4H, m), 2.06 (1H, t, J = 6.5 Hz), 2.21-2.24 (4H, m), |
| | 2.30 (2H, br.s), 3.51 (2H, t, J = 4.5 Hz), 3.55 (2H, t, J = 4.5 Hz), 3.82 (1H, t, J = 7.5 |
| | Hz), 4.04-4.10 (1H, m), 4.18-4.26 (1H, m), 4.37-4.45 (2H, m), 4.64 (1H, dd, J = |
| | 9.2, 5.6 Hz), 4.93 (1H, dd, J = 9.1, 5.2 Hz), 7.11 (1H, d, J = 8.4 Hz), 7.35-7.39 (2H, |
| | m), 7.49 (1H, app.t, J = 2.7 Hz), 8.24 (1H, s), 8.52 (1H, t, J = 5.8 Hz), 8.74 (1H, t, J = |
| | 5.8 Hz), 11.33 (1H, d, J = 5.5 Hz) |
| 10.20 | 0.87-0.92 (6H, m), 1.02-1.20 (2H, m), 1.23-1.45 (4H, m), 2.15-2.24 (4H, m), |
| | 3.31 (2H, br.s), 2.55-2.65 (1H, m), 2.94 (1H, dd, J = 7.5, 4.3 Hz), 3.55 (4H, t, J = 4.5 |
| | Hz), 3.84 (1H, t, J = 7.7 Hz), 4.09 (1H, dd, J = 14.8, 8.7 Hz), 4.20 (1H, dd, J = 14.8, 8.4 |
| | Hz), 4.36-4.46 (2H, m), 4.71 (1H, dd, J = 9.0, 5.5 Hz), 4.88 (1H, dd, J = 8.9, 5.2 Hz), |
| | 7.10 (1H, t, J = 9.7 Hz), 7.34-7.39 (2H, m), 7.49-7.51 (1H, m), 8.17 (1H, s), 8.46 |
| | (1H, t, J = 5.7 Hz), 8.81 (1H, t, J = 5.7 Hz), 11.32 (1H, d, J = 7.1 Hz) |
| 12.01 | 1.08-0.92 (2H, m), 1.29-1.16 (3H, m), 1.39-1.32 (3H, m), 1.48-1.43 (3H, m), 1.73 |
| | (2H, br s), 2.06 (2H, t, J = 7.5 Hz), 2.23 (4H, br s), 2.83 (1H, dd, J = 13.8, 10.0 Hz), |
| | 3.11-3.03 (2H, m), 4.42 (2H, d, J = 5.8 Hz), 4.47-4.37 (2H, m), 4.58 (1H, br s), 6.71 |
| | (2H, br s), 6.82 (1H, d, J = 6.8 Hz), 7.09-7.05 (1H, m), 7.32-7.25 (2H, m), 7.46 (1H, |
| | br s), 7.76 (1H, d, J = 5.7 Hz), 8.12 (1H, d, J = 8.5 Hz), 8.15 (1H, br s), 8.58 (1H, t, J = |
| | 5.9 Hz). |
| 12.02 | 0.95-0.90 (6H, m), 1.50-1.24 (16H, m), 2.29-2.12 (7H, m), 2.63-2.55 (1H, m), |
| | 3.07-3.02 (1H, m), 4.45-4.37 (3H, m), 6.73-6.70 (2H, m), 6.83 (1H, d, J = 5.6 Hz), |
| | 7.33 (1H, dd, J = 1.6, 8.7 Hz), 7.51 (1H, s), 7.77 (1H, d, J = 5.8 Hz), 8.15-8.11 (2H, |
| | m), 8.51 (1H, t, J = 5.9 Hz). |
| 17.01 | 1.27-1.41 (9H, m), 1.68-1.88 (5H, m), 2.04-2.26 (2H, m), 2.55-2.73 (2H, m), |
| | 3.37-3.65 (2H, m), 3.96-4.22 (1H, m), 4.33-4.46 (2H, m), 6.88-6.94 (2H, m), |
| | 7.11-7.30 (7H, m), 7.37-7.39 (1H, m), 7.56-7.59 (1H, m), 7.73 (1H, d, J = 6.0 Hz), |
| | 8.09-8.21 (2H, m). |
| 17.02 | 1.84-1.91 (3H, m), 2.00-2.11 (3H, m), 2.66-2.79 (2H, m), 3.57-3.77 (2H, m), |
| | 4.06-4.14 (1H, m), 4.28-4.31 (1H, m), 4.37-4.51 (2H, m), 7.07-7.37 (7H, m), |
| | 7.63-7.69 (2H, m), 7.78 (1H, s), 8.50-8.59 (3H, m), 8.91 (1H, t, J = 6.0 Hz), 9.23 |
| | (2H, s), 13.32 (1H, s). |
| 17.03 | 1.27-1.29 (2H, m), 1.46-1.51 (1H, m), 1.59-1.62 (2H, m), 1.98-2.01 (2H, m), |
| | 2.27-2.38 (1H, m), 2.68-2.75 (2H, m), 3.09-3.13 (1H, m), 3.59-3.65 (2H, m), |
| | 4.47-4.48 (2H, m), 5.06-5.08 (1H, m), 7.03-7.35 (6H, m), 7.62-7.75 (3H, m), |
| | 8.48-8.57 (3H, m), 8.86 (1H, t, J = 6.0 Hz), 9.11 (2H, s), 13.33 (1H, s) |
| 17.04 | 1.35-1.41 (1H, m), 1.64-1.74 (2H, m), 1.95-2.00 (3H, m), 2.60-2.77 (3H, m), |
| | 2.94-3.26 (1H, m), 3.59-3.89 (2H, m), 4.28-4.63 (4H, m), 7.16-7.35 (5H, m), |
| | 7.63-7.77 (3H, m), 8.38 (2H, s), 8.58 (1H, dd, J = 2.1, 8.6 Hz), 8.73-8.93 (1H, m), |
| | 9.15 (2H, s), 13.41 (1H, s) |
| 17.05 | 1.70-1.65 (1H, m), 1.90-1.84 (1H, m), 2.26-2.18 (1H, m), 2.74-2.58 (3H, m), |
| | 3.27-3.23 (1H, m), 4.09-3.94 (2H, m), 4.50-4.48 (2H, m), 4.83-4.77 (1H, m), |
| | 6.28-6.22 (2H, m), 6.89-6.87 (1H, m), 7.29-7.14 (5H, m), 7.42-7.36 (1H, m), |
| | 7.60-7.55 (1H, m), 7.80 (1H, d, J = 5.9 Hz), 8.13-8.07 (1H, m), 8.37-8.33 (1H, m). |
| 17.06 | 1.85-1.39 (7H, m), 2.20-1.87 (3H, m), 2.71-2.55 (3H, m), 3.51-3.12 (3H, m), |
| | 4.43-3.95 (2H, m), 4.57-4.17 (2H, m), 6.75-6.66 (2H, m), 6.99-6.86 (1H, m), |
| | 7.41-7.05 (6H, m), 7.55-7.50 (1H, m), 7.79-7.75 (1H, m), 8.17-8.08 (1H, m), |
| | 8.53-8.38 (1H, m). |
| 17.07 | (CDCl3): 2.15-1.73 (6H, m), 2.70-2.54 (2H, m), 2.92 (1H, q, J = 7.7 Hz), 3.24 (1H, |
| | dd, J = 4.2, 8.7 Hz), 4.46-4.33 (2H, m), 4.56 (1H, dd, J = 6.3, 14.9 Hz), 5.06-5.00 |
| | (2H, m), 6.20-6.16 (1H, m), 6.95 (1H, d, J = 5.9 Hz), 7.21-7.14 (3H, m), 7.33-7.27 |
| | (3H, m), 7.49 (1H, s), 7.67-7.61 (2H, m), 7.93-7.90 (1H, m). |
| 17.08 | 1.86-1.70 (2H, m), 2.20-2.09 (1H, m), 2.24 (3H, s), 2.48-2.40 (1H, m), 2.75- |
| | 2.57 (2H, m), 3.12-2.95 (2H, m), 4.07-3.83 (1H, m), 4.16 (1H, dd, J = 8.8, 14.9 Hz), |
| | 5.02-4.39 (3H, m), 6.87-6.76 (3H, m), 7.33-6.96 (5H, m), 7.41-7.34 (1H, m), |
| | 7.58-7.55 (1H, m), 7.77 (1H, d, J = 5.8 Hz), 8.22-8.14 (3H, m), 8.90-8.66 (1H, m). |
| 17.09 | 0.98-0.89 (6H, m), 1.83-1.63 (2H, m), 2.23-2.10 (1H, m), 2.78-2.60 (3H, m), |
| | 3.20-3.00 (1H, m), 4.19-3.90 (2H, m), 4.56-4.38 (3H, m), 4.74 (1H, dd, J = 5.4, 8.9 |
| | Hz), 6.87-6.78 (3H, m), 7.38-6.96 (6H, m), 7.59-7.54 (1H, m), 7.77 (1H, d, J = 5.8 |
| | Hz), 8.16-8.10 (1H, m), 8.29 (2H, s), 8.96-8.58 (1H, m). |
| 17.10 | 0.97-0.83 (6H, m), 1.18-1.03 (2H, m), 1.23 (1H, s), 1.51-1.30 (12H, m), 1.99 (1H, |
| | s), 2.34-2.12 (6H, m), 2.91 (0.3H, t, J = 3.9 Hz), 3.15-3.08 (0.7H, m), 3.87-3.81 |
| | (1H, m), 4.25-4.08 (1H, m), 4.54-4.36 (2H, m), 4.73 (0.7H, dd, J = 6.0, 9.6 Hz), 4.93- |
| | 4.88 (0.3H, m), 6.74-6.69 (2H, m), 6.85-6.81 (1H, m), 7.37-7.31 (1H, m), 7.57- |
| | 7.50 (1H, m), 7.79-7.75 (1H, m), 8.17-8.09 (1H, m), 8.63 (0.7H, t, J = 5.9 Hz), 8.91 |
| | (0.3H, t, J = 5.9 Hz). |
| 17.11 | 0.96-0.83 (6H, m), 1.51-1.34 (10H, m), 2.26-1.95 (8H, m), 2.68-2.56 (3H, m), |
| | 3.91-3.78 (0.6H, m), 4.25-4.10 (1.4H, m), 4.55-4.36 (2.3H, m), 4.76-4.74 (0.7H, |
| | m), 6.71 (2H, d, J = 6.8 Hz), 6.85-6.81 (1H, m), 7.34 (1H, t, J = 9.0 Hz), 7.57-7.50 |
| | (1H, m), 7.79-7.75 (1H, m), 8.15-8.09 (1H, m), 8.64-8.56 (0.7H, m), 8.92-8.86 |
| | (0.3H, m). |
| 18.101 | 0.44 (3H, d, J = 6.9 Hz), 0.66 (3H, d, J = 5.4 Hz), 1.11-1.15 (1H, m), 1.51-1.57 (1H, |
| | m), 3.17-3.37 (1H, m), 3.45-3.47 (1H, m), 3.70-3.74 (1H, m), 3.82-3.907 (1H, |
| | m), 4.53 (2H, d, J = 6.1 Hz), 4.66-4.71 (1H, m), 7.18 (1H, d, J = 7.1 Hz), 7.37-7.40 |
| | (1H, m), 7.49-7.60 (5H, m), 7.68-7.71 (2H, m), 7.80 (2H, d, J = 8.4 Hz), 7.93 (1H, d, |
| | J = 7.4 Hz), 8.24 (1H, d, J = 8.4 Hz), 8.39 (1H, d, J = 3.0 Hz), 8.58 (1H, d, J = 8.7 Hz), |
| | 9.14 (1H, t, J = 6.0z Hz), 9.22 (1H, d, J = 8.1 Hz), 13.38 (1H, br.s). |
| 18.102 | 1.80-1.96 (2H, m), 2.54-2.69 (2H, m), 3.80-3.86 (2H, m), 3.89-3.99 (1H, m), |
| | 4.27-4.38 (2H, m), 4.41-4.47 (2H, m), 6.82 (1H, d, J = 5.8 Hz), 6.84 (2H, s), 7.15- |
| | 7.18 (3H, m), 7.21-7.29 (2H, m), 7.31-7.38 (6H, m), 7.52 (1H, s), 7.63 (1H, d, J = |
| | 8.1 Hz), 7.74 (1H, d, J = 5.8 Hz), 8.12 (1H, d, J = 8.6 Hz), 8.41 (1H, t, J = 5.8 Hz), 8.46 |
| | (1H, t, J = 6.0H z). |
| 18.103 | 0.88 (3H, d, J = 7.0 Hz), 0.92 (3H, d, J = 7.3 Hz), 1.16-1.24 (2H, m), 1.49-1.56 |
| | (2H, m), 2.02 (1H, br.s), 2.77 (6H, s), 3.91-3.96 (1H, m), 4.52 (2H, d, J = 4.8 Hz), |
| | 7.16 (1H, d, J = 6.9 Hz), 7.64-7.68 (1H, m), 7.79 (1H, s), 8.48 (1H, d, J = 8.6 Hz), |
| | 8.70-8.80 (1H, m), 8.94 (2H, br.s), 9.46 (1H, br.s), 13.02 (1H, br.s). |
| 18.104 | 2.02-2.09 (1H, m), 2.14-2.19 (1H, m), 2.54-2.59 (1H, m), 2.82 (6H, s), 3.85 (2H, |
| | q, J = 3.2 Hz), 3.99 (2H, t, J = 5.3 Hz), 4.52 (2H, d, J = 5.9 Hz), 7.17-7.21 (3H, m), |
| | 7.26 (1H, d, J = 7.2 Hz), 7.66-7.69 (2H, m), 7.81 (1H, s), 8.49 (1H, d, J = 8.6 Hz), |
| | 8.81 (1H, t, J = 5.8 Hz), 9.03 (2H, br.s), 9.13 (1H, s), 9.97 (1H, br.s), 13.19 (1H, br.s). |
| 18.105 | 1.33 (6H, s), 4.40 (2H, d, J = 5.8 Hz), 6.77 (2H, s), 6.88 (1H, d, J = 5.8 Hz), 7.42 (1H, |
| | d, J = 8.8 Hz), 7.60 (1H, s), 7.79-7.68 (2H, m), 7.82 (1H, d, J = 5.8 Hz), 7.99-7.96 |
| | (1H, m), 8.13-8.07 (2H, m), 8.16 (3H, dd, J = 4.4, 8.2 Hz), 8.31 (1H, s), 8.34 (1H, dd, |
| | J = 5.9, 5.9 Hz), 8.52 (1H, s). |
| 18.106 | 3.23 (4H, t, J = 4.9 Hz), 3.77-3.72 (4H, m), 3.95-3.91 (2H, m), 4.46-4.43 (2H, m), |
| | 6.78-6.74 (2H, m), 6.85 (1H, d, J = 5.8 Hz), 7.02-6.97 (2H, m), 7.37 (1H, dd, J = 1.7, |
| | 8.6 Hz), 7.54 (1H, s), 7.84-7.77 (3H, m), 8.20-8.12 (2H, m), 8.61-8.47 (2H, m). |
| 18.107 | 4.13-4.10 (2H, m), 4.49-4.46 (2H, m), 6.73 (2H, s), 6.87 (1H, d, J = 5.6 Hz), 7.41- |
| | 7.37 (1H, m), 7.57-7.56 (1H, m), 7.80-7.73 (2H, m), 7.93-7.88 (1H, m), 8.22- |
| | 8.10 (4H, m), 8.68-8.59 (2H, m), 9.18 (1H, t, J = 5.9 Hz). |
| 18.108 | 4.00 (2H, d, J = 5.9 Hz), 4.46 (2H, d, J = 6.0 Hz), 6.72 (2H, s), 6.86 (1H, d, J = 5.6 Hz), |
| | 7.40-7.36 (1H, m), 7.56 (1H, s), 7.81-7.78 (3H, m), 7.98-7.94 (2H, m), 8.16-8.06 |
| | (3H, m), 8.59 (1H, t, J = 6.0 Hz), 8.70-8.68 (2H, m), 8.98 (1H, t, J = 5.9 Hz). |
| 24.01 | 3.39-3.44 (2H, m), 4.20 (1H, br.s), 4.47 (2H, t, J = 5.1 Hz), 7.14 (1H, d, J = 7.0 Hz), |
| | 7.37-7.44 (2H, m), 7.54 (1H, dd, J = 8.7, 1.5 Hz), 7.59 (1H, s), 7.69 (1H, d, J = 6.9 |
| | Hz), 7.71 (1H, s), 8.00-8.05 (2H, m), 8.53 (1H, d, J = 8.6 Hz), 8.60 (3H, s), 9.18 (2H, |
| | s), 9.38 (1H, t, J = 5.9 Hz), 13.39 (1H, s). |
| 24.02 | 3.14-3.19 (2H, m), 4.21 (1H, s), 4.48 (2H, d, J = 4.7 Hz), 7.10-7.14 (4H, m), 7.17 |
| | (1H, d, J = 7.0 Hz), 7.33-7.37 (1H, m), 7.51-7.54 (1H, m), 7.70 (1H, d, J = 7.0 Hz), |
| | 7.77 (1H, s), 8.57 (1H, s), 9.28 (2H, br.s), 9.50 (1H, t, J = 5.9 Hz), 13.54 (1H, br.s). |
| 24.03 | 2.55 (2H, d, J = 6.2 Hz), 2.78-2.84 (1H, m), 3.01-3.06 (1H, m), 3.70-3.72 (1H, m), |
| | 4.41-4.52 (2H, m), 7.18 (1H, d, J = 7.0 Hz), 7.21-7.37 (5H, m), 7.63 (1H, dd, J = 8.6, |
| | 1.4 Hz), 7.69 (1H, d, J = 7.1 Hz), 7.77 (1H, s), 8.13 (3H, s), 8.55 (1H, d, J = 8.6 Hz), |
| | 8.90 (1H, t, J = 5.9 Hz), 9.06 (2H, s), 13.29 (1H, s). |
| 27.01 | 1.15-1.25 (7H, m), 1.76-1.82 (1H, m), 1.89-1.99 (2H, m), 2.05-2.09 (2H, m), |
| | 2.12-2.19 (1H, m), 2.65-2.73 (2H, m), 3.08-3.17 (1H, m), 3.47-3.51 (1H, m), |
| | 3.62-3.68 (2H, m), 4.28 (1H, d, J = 6.8 Hz), 4.34 (1H, dd, J = 3.8, 8.6 Hz), 4.40 (2H, |
| | d, J = 5.8 Hz), 7.20-7.24 (3H, m), 7.30-7.34 (2H, m), 7.67 (1H, d, J = 2.7 Hz), 7.79 |
| | (1H, d, J = 1.6 Hz), 8.23 (1H, d, J = 1.9 Hz), 8.54 (1H, t, J = 5.8 Hz), 11.92 (1H, s) |
| 27.03 | 0.79 (3H, d, J = 6.2 Hz), 0.84 (3H, d, J = 6.0 Hz), 0.92-1.00 (1H, m), 1.00-1.11 (1H, |
| | m), 1.21-1.30 (4H, m), 1.32-1.39 (2H, m), 1.41-1.49 (4H, m), 1.62 (1H, br s), 2.05 |
| | (2H, t, J = 7.5 Hz), 2.25 (4H, br.s), 2.31-2.38 (1H, m), 2.83 (1H, dd, J = 10.0, 13.7 |
| | Hz), 2.98 (2H, dt, J = 5.2, 13.1 Hz), 4.37 (2H, d, J = 5.7 Hz), 4.60 (1H, ddd, J = 9.2, 9.2, |
| | 4.8 Hz), 7.02-7.07 (2H, m), 7.21-7.30 (2H, m), 7.32-7.38 (2H, m), 7.49 (1H, s), |
| | 8.13 (1H, d, J = 8.8 Hz), 8.41 (1H, t, J = 5.8 Hz), 11.32 (1H, s) |
| 27.04 | 0.84-0.75 (9H, m), 1.28-1.21 (6H, m), 1.47-1.31 (6H, m), 2.08-2.00 (2H, m), |
| | 2.28-2.16 (2H, m), 2.37-2.29 (1H, m), 2.87-2.78 (1H, m), 3.02-2.92 (2H, m), |
| | 4.48-4.34 (2H, m), 4.63-4.54 (1H, m), 7.04-7.02 (1H, m), 7.29-7.20 (2H, m), |
| | 7.67-7.65 (1H, m), 7.79-7.77 (1H, m), 8.21-8.11 (2H, m), 8.48 (1H, t, J = 5.7 Hz), |
| | 11.95 (1H, s). |
| 27.05 | 0.82 (2H, q, J = 10.2 Hz), 1.10-0.94 (6H, m), 1.49-1.21 (14H, m), 1.76-1.71 (1H, |
| | m), 2.07-1.97 (3H, m), 2.21-2.17 (4H, m), 2.83 (1H, dd, J = 10.0, 13.8 Hz), 3.03- |
| | 2.93 (2H, m), 4.47-4.35 (2H, m), 4.63-4.55 (1H, m), 7.04-7.01 (1H, m), 7.30- |
| | 7.20 (2H, m), 7.66 (1H, s), 7.80-7.78 (1H, m), 8.11-8.07 (1H, m), 8.21 (1H, d, J = |
| | 2.0 Hz), 8.53-8.47 (1H, m), 11.96-11.93 (1H, m). |
| 27.06 | 1.28-0.97 (6H, m), 1.40-1.28 (3H, m), 1.49-1.41 (4H, m), 2.06 (2H, t, J = 7.5 Hz), |
| | 2.23-2.19 (4H, m), 2.80 (1H, dd, J = 9.7, 13.6 Hz), 3.11-2.96 (2H, m), 3.42-3.34 |
| | (1H, m), 4.54-4.38 (3H, m), 7.02-6.98 (1H, m), 7.28-7.19 (2H, m), 7.66 (1H, s), |
| | 7.77 (1H, d, J = 2.0 Hz), 8.21-8.19 (2H, m), 8.56-8.50 (1H, m), 11.96 (1H, s). |
| 27.07 | 0.88-0.77 (2H, m), 1.11-1.07 (1H, m), 1.22 (6H, dd, J = 6.4, 18.3 Hz), 1.41-1.35 |
| | (1H, m), 1.83-1.51 (9H, m), 2.86-2.75 (4H, m), 3.07 (2H, dd, J = 3.9, 13.6 Hz), 3.85- |
| | 3.78 (1H, m), 4.47 (2H, d, J = 6.4 Hz), 4.64-4.57 (1H, m), 6.97-6.94 (2H, m), 7.18- |
| | 7.11 (1H, m), 7.44-7.37 (3H, m), 8.77-8.72 (1H, m), 8.89 (1H, t, J = 5.8 Hz), 9.16 |
| | (2H, d, J = 8.3 Hz), 10.15 (1H, s). |
| 27.08 | 0.94-0.82 (6H, m), 1.48-1.38 (1H, m), 1.77-1.56 (3H, m), 2.19-2.02 (1H, m), |
| | 2.44-2.24 (1H, m), 2.64-2.54 (1.3H, m), 2.75-2.67 (0.7H, m), 2.95 (0.3H, d, J = 1.3 |
| | Hz), 3.06 (0.7H, s), 3.93-3.79 (1.3H, m), 4.06-3.98 (0.7H, m), 4.51-4.36 (2H, m), |
| | 4.67 (0.7H, dd, J = 5.4, 9.0 Hz), 4.87 (0.3H, dd, J = 5.4, 9.0 Hz), 6.98 (1H, d, J = 7.2 |
| | Hz), 7.30-7.08 (4H, m), 7.66 (1H, s), 7.82 (1H, d, J = 2.0 Hz), 8.27-8.22 (1H, m), |
| | 8.55-8.49 (0.7H, m), 8.86 (0.3H, t, J = 5.7 Hz), 11.96-11.89 (1H, m). |
| 32.01 | 2.04-2.21 (1H, m), 2.68 (3H, s), 3.07 (1H, t, J = 7.4 Hz), 3.16 (1H, t, J = 7.4 Hz), 3.53- |
| | 3.60 (2H, m), 3.68 (2H, d, J = 7.5 Hz), 3.78-3.83 (1H, m), 4.38 (1H, d, J = 5.0 Hz), |
| | 4.47 (1H, d, J = 5.7 Hz), 6.79 (1H, d, J = 5.0 Hz), 6.90 (1H, d, J = 6.0 Hz), 7.29-7.40 |
| | (1H, m), 7.44-7.55 (2H, m), 7.75 (1H, dd, J = 17.2, 5.8 Hz), 7.82 (1H, d, J = 8.6 Hz), |
| | 7.95 (1H, d, J = 8.6 Hz), 8.10-8.19 (2H, m), 8.33 (1H, d, J = 8.4 Hz), 8.54-8.70 (1H, |
| | br.m) |
| 32.02 | 0.93 (3H, d, J = 6.3 Hz), 0.99 (3H, d, J = 6.2 Hz), 1.05-1.17 (1H, m), 1.21-1.66 (4H, |
| | m), 1.75-2.33 (5H, m), 2.87-3.20 (3H, m), 3.43-3.49 (1H, m), 3.55 (2H, s), 4.32- |
| | 4.47 (3H, m), 6.69 (2H, s), 6.83-6.85 (1H, m), 7.32 (1H, dd, J = 8.5, 1.5 Hz), 7.56 |
| | (1H, s), 7.75 (1H, d, J = 5.7 Hz), 8.09 (1H, d, J = 8.6 Hz), 8.35 (1H, t, J = 5.9 Hz). |
| 32.03 | 0.89 (1H, d, J = 6.6 Hz), 1.04 (3H, d, J = 6.2 Hz), 1.06-1.76 (14H, m), 3.32-3.40 |
| | (1H, m), 3.88-4.32 (6H, m), 4.45-4.64 (2H, m), 7.15-7.21 (1H, m), 7.60-7.72 (3H, |
| | m), 8.48-8.52 (1H, m), 8.70-8.93 (2H, m), 9.19 (2H, s), 13.53 (1H, s). |
| 32.04 | 0.90 (3H, d, J = 6.5 Hz), 1.0-1.11 (1H, m), 1.16 (3H, t, J = 6.3 Hz), 1.27-1.31 (1H, |
| | m), 1.45-1.55 (3H, m), 1.61-1.79 (3H, m), 3.07-3.17 (1H, m), 3.67-3.74 (1H, m), |
| | 4.28 (1H, d, J = 17.6 Hz), 4.45-4.50 (1H, m), 4.57-4.63 (1H, m), 5.50 (1H, d, J = 9.5 |
| | Hz), 7.13-7.16 (2H, m), 7.25-7.29 (1H, m), 7.35 (1H, d, J = 7.4 Hz), 7.69 (2H, d, J = |
| | 6.9 Hz), 7.83 (1H, s), 8.04 (1H, d, J = 8.0 Hz), 8.48 (1H, d, J = 8.6 Hz), 9.07 (3H, br.s), |
| | 9.31 (1H, d, J = 5.6 Hz). |
| 32.06 | 1.09 (3H, d, J = 6.1 Hz), 1.05 (3H, d, J = 6.4 Hz), 1.20-1.24 (1H, m), 1.52-1.76 (4H, |
| | m), 1.96-2.03 (1H, m), 2.07-2.25 (1H, m), 3.07-3.20 (1H, m), 3.36-3.89 (5H, m), |
| | 3.91-3.95 (2H, m), 4.50 (2H, d, J = 5.9 Hz), 7.21 (1H, dd, J = 2.5, 7.0 Hz), 7.64-7.70 |
| | (2H, m), 7.77 (1H, s), 8.51 (1H, dd, J = 2.5, 8.6 Hz), 8.79-8.89 (2H, m), 9.14 (2H, s), |
| | 13.42 (1H, s). |
| 32.07 | 1.08 (3H, d, J = 5.8 Hz), 1.10 (3H, d, J = 6.2 Hz), 1.20-1.23 (1H, m), 1.52-1.77 (4H, |
| | m), 1.91-2.05 (1H, m), 2.07-2.24 (1H, m), 3.09-3.20 (1H, m), 3.37-3.76 (5H, m), |
| | 3.91-3.94 (2H, m), 4.50 (2H, dd, J = 5.6, 9.2 Hz), 7.21 (1H, dd, J = 2.9, 7.0 Hz), 7.65- |
| | 7.69 (2H, m), 7.77 (1H, s), 8.50 (1H, dd, J = 2.2, 8.6 Hz), 8.78-8.86 (2H, m), 9.04 |
| | (2H, s), 13.20 (1H, s). |
| 32.08 | 1.07 (3H, d, J = 2.9 Hz), 1.10 (3H, d, J = 2.9 Hz), 1.16-1.21 (1H, m), 1.43-1.69 (4H, |
| | m), 1.78-1.82 (1H, m), 1.98-2.00 (1H, m), 2.91 (1H, t, J = 12.5 Hz), 3.08 (1H, t, J = |
| | 11.5 Hz), 3.25-3.30 (1H, m), 3.46-4.04 (5H, m), 4.07-4.18 (1H, m), 4.37-4.49 |
| | (3H, m), 7.19 (1H, t, J = 7.2 Hz), 7.63-7.69 (2H, m), 7.75 (1H, s), 8.49 (1H, d, J = 8.6 |
| | Hz), 8.68-8.73 (1H, m), 8.83 (1H, s), 9.06 (2H, s), 13.32 (1H, s). |
| 32.09 | 1.10 (6H, d, J = 6.7 Hz), 1.2-1.30 (1H, m), 1.50-1.60 (6H, m), 1.65-1.70 (2H, m), |
| | 2.65-2.71 (2H, m), 2.89-3.04 (3H, m), 3.51-3.61 (1H, m), 4.29-4.35 (1H, m), |
| | 4.43-4.47 (1H, m), 5.00-5.05 (1H, m), 6.77 (1H, d, J = 5.9 Hz), 6.82 (1H, s), 7.20- |
| | 7.24 (5H, m), 7.38 (1H, s), 7.74 (1H, d, J = 5.9 Hz), 8.08 (1H, d, J = 8.6 Hz), 8.51 (1H, |
| | br.s). |
| 32.12 | (CDCl3): 1.10 (6H, dd, J = 6.3, 9.6 Hz), 1.43-1.19 (4H, m), 1.67-1.62 (2H, m), 2.72- |
| | 2.66 (2H, m), 3.36-3.28 (3H, m), 4.20-4.15 (2H, m), 4.41 (1H, t, J = 8.8 Hz), 4.65- |
| | 4.56 (3H, m), 5.11 (2H, s), 5.87 (1H, t, J = 5.6 Hz), 7.02-6.99 (1H, m), 7.40 (1H, dd, |
| | J = 1.8, 8.5 Hz), 7.55 (1H, d, J = 20.8 Hz), 7.80-7.76 (1H, m), 7.97-7.94 (1H, m). |
| 32.13 | 1.17-0.73 (15H, m), 1.67-1.17 (7H, m), 3.02-2.88 (2H, m), 3.80-3.11 (4H, m), |
| | 4.6-4.2 (3H, m), 6.17 (2H, s), 6.93-6.83 (1H, m), 7.45-7.33 (1H, m), 7.63-7.51 |
| | (1H, m), 7.82-7.72 (1H, m), 8.21-7.88 (2H, m) |
| 32.14 | 1.01-0.94 (6H, m), 1.17-1.04 (2H, m), 1.36-1.24 (1H, m), 1.53-1.37 (3H, m), |
| | 1.72-1.53 (2H, m), 1.92-1.73 (2H, m), 2.62-2.54 (1H, m), 2.81-2.65 (1H, m), |
| | 3.02-2.85 (2H, m), 3.18-3.08 (1H, m), 3.33-3.28 (2H, m), 3.62-3.36 (1H, m), |
| | 4.47-4.31 (3H, m), 6.72 (2H, d, J = 3.4 Hz), 6.85 (1H, dd, J = 3.0, 5.6 Hz), 7.36-7.31 |
| | (1H, m), 7.50 (1H, d, J = 1.5 Hz), 7.78 (1H, dd, J = 1.0, 5.8 Hz), 8.16-8.12 (1H, m), |
| | 8.59-8.50 (1H, m). |
| 39.01 | 0.92 (6H, br.s), 1.04-1.17 (4H, m), 1.20-1.24 (2H, m), 1.55-1.58 (2H, m), 1.74- |
| | 1.77 (2H, m), 2.34-2.46 (2H, m), 2.94 (1H, br.s), 3.35-3.38 (2H, m), 4.39 (2H, d, J = |
| | 5.8 Hz), 6.83 (1H, s), 6.86 (1H, d, J = 5.9 Hz), 7.35 (1H, dd, J = 1.6, 8.6 Hz), 7.51 (1H, |
| | s), 7.75 (1H, d, J = 5.9 Hz), 7.84 (1H, br.s), 8.14 (1H, d, J = 8.6 Hz), 8.50 (1H, t, J = 5.8 |
| | Hz). |
| 39.02 | 0.96 (6H, t, J = 7.3 Hz), 1.15-1.18 (2H, m), 1.26 (3H, d, J = 7.0 Hz), 1.29-1.35 (1H, |
| | m), 1.50-1.62 (3H, m), 3.00 (2H, s), 3.30-3.41 (2H, m), 4.39-4.46 (3H, m), 6.74 |
| | (2H, s), 6.83 (1H, d, J = 5.8 Hz), 7.31 (1H, dd, J = 8.6, 1.4 Hz), 7.49 (1H, s), 7.76 (1H, |
| | d, J = 5.7 Hz), 8.02 (1H, s), 8.13 (1H, d, J = 8.6 Hz), 8.64 (1H, t, J = 5.7 Hz). |
| 39.03 | 1.13 (3H, d, J = 6.6 Hz), 1.27 (3H, d, J = 6.3 Hz), 1.35-1.42 (2H, m), 1.52-1.67 (2H, |
| | m), 1.74-1.81 (2H, m), 3.89-3.98 (4H, m), 4.08 (1H, br.s), 4.52 (2H, d, J = 5.8 Hz), |
| | 7.17 (1H, d, J = 7.0 Hz), 7.64-7.68 (2H, m), 7.77 (1H, s), 7.98 (1H, d, J = 8.4 Hz), |
| | 8.49 (1H, d, J = 8.6 Hz), 8.75 (1H, t, J = 6.3 Hz), 8.91 (1H, d, J = 5.6 Hz), 9.05 (1H, |
| | br.s), 13.09 (1H, brs). |
| 39.04 | 0.85 (2H, d, J = 6.4 Hz), 1.00-1.04 (3H, m), 1.11 (1H, d, J = 6.1 Hz), 1.21-1.66 (6H, |
| | m), 2.68-2.73 (1H, m), 2.84-2.87 (1H, m), 3.17-3.34 (3H, m), 3.75-3.88 (1H, m), |
| | 4.47 (2H, s), 4.48 (2H, s), 7.18-7.38 (7H, m), 7.67 (2H, d, J = 6.3 Hz), 7.78 (1H, s), |
| | 8.50 (1H, d, J = 8.6 Hz), 8.59 (1H, d, J = 8.4 Hz), 8.70-8.86 (2H, m), 9.11 (2H, s), |
| | 13.34 (1H, s). |
| 39.05 | 0.98 (3H, d, J = 6.3 Hz), 1.10 (3H, d, J = 6.2 Hz), 1.19-1.21 (1H, m), 1.25-1.32 (3H, |
| | m), 1.45-1.61 (2H, m), 1.57-1.60 (2H, m), 2.06-2.18 (1H, m), 2.33-2.49 (2H, m), |
| | 2.95-2.98 (1H, m), 3.03 (1H, d, J = 7.2 Hz), 3.94-3.99 (1H, m), 4.41-4.54 (6H, m), |
| | 6.70 (1H, s), 6.73 (1H, d, J = 5.8 Hz), 7.24-7.26 (6H, m), 7.27-7.35 (1H, m), 7.50 |
| | (1H, s), 7.74 (1H, d, J = 5.8 Hz), 8.10-8.14 (1H, d, J = 8.6 Hz), 8.54 (1H, t, J = 5.8 Hz). |
| 39.06 | 1.11-1.19 (6H, m), 1.24-1.29 (4H, m), 1.64-1.78 (4H, m), 2.33 (3H, d, J = 1.7 Hz), |
| | 2.54 (3H, d, J = 1.7 Hz), 4.19-4.34 (2H, m), 4.52 (2H, d, J = 5.2 Hz), 6.95 (1H, s), |
| | 7.08 (1H, s), 7.20 (1H, s), 6.67-7.72 (2H, m), 7.77(1H, s), 8.44-8.52 (1H, m), 8.59- |
| | 8.65 (1H, m), 8.90 (1H, s). |
| 39.07 | 0.95-0.98 (6H, m), 1.19-1.44 (5H, m), 1.61-1.78 (1H, m), 2.33-2.47 (2H, m), |
| | 2.67-2.93 (2H, m), 3.96-4.29 (2H, m), 4.47 (2H, d, J = 5.9 Hz), 6.03 (1H, d, J = 7.9 |
| | Hz), 7.16-7.28 (7H, m), 7.64-7.66 (2H, m), 7.76 (1H, s), 8.48 (1H, d, J = 8.6 Hz), |
| | 8.57 (1H, t, J = 5.8 Hz), 8.96 (2H, s), 12.98 (1H, s). |
| 39.08 | 1.09 (4H, d, J = 6.6 Hz), 1.22 (2H, d, J = 6.3 Hz), 1.26-1.42 (2H, m), 1.45 (3H, s), |
| | 1.47 (3H, s), 1.50-1.79 (5H, m), 3.81-3.86 (2H, m), 4.00-4.01 (1H, d, J = 2.5 Hz), |
| | 4.46 (2H, d, J = 5.9 Hz), 7.14-7.18 (1H, m), 7.61-7.68 (2H, m), 7.75 (1H, d, J = 7.8 |
| | Hz), 8.44-8.52 (2H, m), 8.67-8.87 (2H, m), 9.03 (2H, s), 13.16 (1H, s). |
| 39.09 | 1.03-0.94 (9H, m), 1.40-1.19 (6H, m), 1.50 (2H, d, J = 11.4 Hz), 1.63 (1H, d, J = |
| | 12.4 Hz), 3.18 (2H, s), 4.48 (2H, d, J = 5.8 Hz), 6.74 (2H, s), 6.86 (1H, d, J = 5.6 Hz), |
| | 7.38 (1H, d, J = 8.6 Hz), 7.56 (1H, s), 7.81 (1H, d, J = 5.6 Hz), 7.96 (1H, dd, J = 5.9, 5.9 |
| | Hz), 8.16 (1H, d, J = 8.6 Hz), 8.26 (1H, s). |
| 39.10 | 1.01-0.98 (6H, m), 1.33-1.11 (3H, m), 1.49 (2H, dd, J = 1.6, 11.0 Hz), 1.64-1.56 |
| | (1H, m), 1.96-1.78 (2H, m), 2.23-2.13 (2H, m), 2.65-2.55 (4H, m), 3.12-3.10 (2H, |
| | m), 4.43 (2H, d, J = 5.9 Hz), 6.70 (2H, s), 6.82-6.80 (1H, m), 7.32 (1H, dd, J = 1.6, |
| | 8.7 Hz), 7.49 (1H, s), 7.78-7.76 (1H, m), 7.99 (1H, t, J = 6.0 Hz), 8.12-8.09 (1H, m), |
| | 8.19 (1H, s). |
| 39.11 | 1.01 (6H, d, J = 6.3 Hz), 1.33-1.10 (3H, m), 1.53-1.49 (2H, m), 1.60 (1H, dd, J = |
| | 2.9, 9.2 Hz), 2.10-1.97 (4H, m), 2.65-2.56 (2H, m), 3.16 (2H, s), 3.58-3.45 (2H, m), |
| | 3.78-3.71 (2H, m), 4.43 (2H, d, J = 5.9 Hz), 6.70 (2H, s), 6.82 (1H, d, J = 5.6 Hz), |
| | 7.33 (1H, dd, J = 1.6, 8.6 Hz), 7.51 (1H, s), 7.81-7.76 (2H, m), 8.12-8.01 (2H, m). |
| 43.01 | 2.07-1.85 (3H, m), 2.47-2.19 (2H, m), 3.62-3.48 (2H, m), 4.01-3.87 (1H, m), |
| | 4.63-4.44 (3H, m), 6.78 (2H, s), 6.85 (1H, t, J = 5.2 Hz), 7.37-7.34 (6H, m), 7.55 |
| | (1H, d, J = 12.0 Hz), 7.80-7.77 (1H, m), 8.19-8.14 (1H, m), 8.89-8.59 (1H, m). |
| 43.02 | 0.85-0.81 (1H, m), 1.05-0.92 (6H, m), 1.44-1.18 (4H, m), 1.65-1.50 (3H, m), |
| | 2.37-2.18 (2H, m), 3.18 (2H, dd, J = 10.7, 17.1 Hz), 3.70-3.45 (1H, m), 4.12-4.06 |
| | (1H, m), 4.59-4.38 (4H, m), 6.80 (2H, s), 6.86 (1H, d, J = 5.9 Hz), 7.38-7.21 (6H, |
| | m), 7.57 (1H, d, J = 11.8 Hz), 7.77 (1H, t, J = 5.8 Hz), 8.23-8.10 (3H, m), 8.87-8.49 |
| | (1H, m). |
| 43.03 | 1.95-1.56 (2H, m), 2.45-2.19 (3H, m), 2.77-2.62 (2H, m), 3.27 (1H, t, J = 9.7 Hz), |
| | 3.59-3.46 (1H, m), 3.68 (1H, dd, J = 4.4, 8.0 Hz), 4.03-3.80 (1H, m), 4.91-4.37 |
| | (4H, m), 6.87-6.75 (3H, m), 7.39-6.97 (11H, m), 7.58-7.54 (1H, m), 7.79-7.75 |
| | (1H, m), 8.16-8.12 (1H, m), 8.90-8.59 (1H, m). |
| 43.04 | 1.52-1.24 (6H, m), 2.36-2.11 (2H, m), 3.83-3.58 (2H, m), 4.61-4.35 (4H, m), |
| | 5.26-5.25 (1H, m), 6.73 (2H, s), 6.88-6.82 (1H, m), 7.40-7.19 (6H, m), 7.61-7.58 |
| | (1H, m), 7.79-7.76 (1H, m), 8.16-8.12 (1H, m), 8.44-8.39 (1H, m). |
| 43.05 | 1.04-0.86 (2H, m), 1.61-1.40 (3H, m), 1.88-1.81 (1H, m), 2.29-2.11 (2H, m), |
| | 2.46-2.33 (5H, m), 2.97-2.86 (3H, m), 3.26 (1H, dd, J = 3.6, 10.4 Hz), 3.61-3.59 |
| | (1H, m), 4.59-4.38 (2H, m), 6.73 (2H, s), 6.85 (1H, d, J = 5.8 Hz), 7.33-7.29 (5H, |
| | m), 7.41-7.35 (1H, m), 7.54-7.52 (1H, m), 7.79 (1H, d, J = 5.8 Hz), 8.18-8.14 (1H, |
| | m), 8.34 (1H, t, J = 6.1 Hz). |
| 43.06 | 1.83-1.56 (3H, m), 2.10-1.98 (1H, m), 2.37-2.20 (2H, m), 2.73-2.63 (1H, m), |
| | 3.07-2.96 (1H, m), 3.52-3.41 (1H, m), 3.84-3.63 (2H, m), 4.07-3.95 (1H, m), |
| | 4.51-4.43 (2H, m), 4.64-4.56 (1H, m), 6.72 (2H, d, J = 7.3 Hz), 6.89-6.82 (1H, m), |
| | 7.37-7.34 (7H, m), 7.54 (1H, d, J = 13.1 Hz), 7.80-7.77 (1H, m), 8.17-8.11 (1H, |
| | m), 8.91-8.53 (1H, m). |
| 43.07 | 1.25 (6H, d, J = 14.9 Hz), 2.34-2.12 (6H, m), 3.92-3.80 (1H, m), 4.48-4.42 (3H, |
| | m), 4.66-4.60 (1H, m), 6.77-6.73 (2H, m), 6.85 (1H, d, J = 5.8 Hz), 7.28-7.23 (1H, |
| | m), 7.40-7.32 (5H, m), 7.60 (1H, s), 7.78 (1H, d, J = 5.8 Hz), 8.16-8.12 (1H, m), |
| | 8.30 (3H, s), 8.47-8.45 (1H, m). |
| 44.01 | 0.84-0.93 (10H, m), 1.50-1.60 (2H, m), 2.87-2.94 (2H, m), 3.04-3.08 (2H, m), |
| | 4.43 (2H, d, J = 5.8 Hz), 4.62 (1H, br.s), 6.84 (1H, d, J = 5.9 Hz), 6.94 (1H, br.s), 7.06 |
| | (1H, br.s), 7.26-7.3 (4H, m), 7.49 (1H, s), 7.69 (1H, d, J = 7.0 Hz), 7.76 (1H, d, J = |
| | 5.9 Hz), 8.15 (1H, d, J = 8.6 Hz), 8.67 (1H, br.s). |
| 45.01 | 1.33-1.30 (3H, m), 1.81-1.71 (1H, m), 2.59-2.52 (1H, m), 2.71 (1H, t, J = 10.3 |
| | Hz), 3.25-3.09 (1H, m), 3.43-3.30 (1H, m), 3.88-3.81 (1H, m), 4.48-4.34 (3H, m), |
| | 6.85-6.76 (3H, m), 7.27-7.15 (5H, m), 7.37-7.32 (1H, m), 7.52 (1H, s), 7.77-7.75 |
| | (1H, m), 8.17-8.11 (1H, m), 8.20 (2H, s), 8.36-8.31 (1H, m), 8.65-8.60 (1H, m). |
| 45.02 | 1.33-1.30 (3H, m), 4.20-4.03 (2H, m), 4.48-4.33 (3H, m), 4.63-4.59 (1H, m), |
| | 6.39-6.32 (1H, m), 6.77-6.73 (2H, m), 6.82-6.78 (1H, m), 7.39-7.28 (4H, m), |
| | 7.45-7.40 (2H, m), 7.49-7.45 (1H, m), 7.73 (1H, d, J = 5.8 Hz), 8.13 (1H, d, J = 8.8 |
| | Hz), 8.20 (2H, s), 8.30 (1H, d, J = 7.6 Hz), 8.63 (1H, t, J = 6.0 Hz). |
| 45.03 | 1.33-1.27 (3H, m), 1.80-1.71 (1H, m), 2.70-2.62 (2H, m), 3.20-3.11 (1H, m), |
| | 3.31-3.27 (2H, m), 3.81-3.74 (1H, m), 4.47-4.36 (3H, m), 6.74-6.70 (2H, m), |
| | 6.84 (1H, dd, J = 5.8, 11.7 Hz), 7.36-7.15 (6H, m), 7.55-7.50 (1H, m), 7.77 (1H, t, |
| | J = 6.1 Hz), 8.16-8.12 (1H, m), 8.29-8.25 (1H, m), 8.65-8.58 (1H, m). |
| 45.04 | 1.34-1.31 (3H, m), 2.79-2.68 (2H, m), 2.98-2.91 (1H, m), 3.52-3.47 (1H, m), |
| | 3.98-3.88 (2H, m), 4.47-4.37 (3H, m), 6.74-6.71 (2H, m), 6.85 (1H, d, J = 5.5 Hz), |
| | 7.07-7.01 (1H, m), 7.13-7.10 (3H, m), 7.36-7.32 (1H, m), 7.51 (1H, s), 7.78 (1H, |
| | d, J = 5.8 Hz), 8.16-8.10 (2H, m), 8.57-8.52 (1H, m). |
| 45.05 | 1.35-1.31 (3H, m), 4.42-4.18 (6H, m), 4.86-4.83 (1H, m), 6.84-6.80 (3H, m), |
| | 7.33-7.19 (4H, m), 7.48-7.44 (2H, m), 7.78-7.76 (1H, m), 8.15-8.09 (1H, m), |
| | 8.45-8.41 (1H, m), 8.66-8.60 (1H, m). |
| 54.01 | 0.92-0.89 (2H, m), 1.36-1.25 (2H, m), 1.69-1.58 (1H, m), 2.05-1.80 (3H, m), |
| | 2.70-2.55 (2H, m), 3.17 (1H, dd, J = 5.0, 8.1 Hz), 4.46-4.42 (2H, m), 6.71-6.68 |
| | (2H, m), 6.83-6.80 (1H, m), 7.19-7.15 (3H, m), 7.36-7.24 (3H, m), 7.51-7.50 (1H, |
| | m), 7.78-7.75 (1H, m), 8.13-8.09 (1H, m), 8.26 (1H, t, J = 6.2 Hz), 8.45-8.42 (1H, |
| | m). |
| 54.02 | 1.81-1.70 (1H, m), 2.00-1.86 (3H, m), 2.20-2.09 (2H, m), 2.75-2.55 (4H, m), |
| | 3.36 (1H, dd, J = 5.4, 7.7 Hz), 4.45-4.40 (2H, m), 6.75-6.71 (2H, m), 6.81-6.78 |
| | (1H, m), 7.35-7.14 (6H, m), 7.49 (1H, s), 7.75 (1H, d, J = 5.8 Hz), 8.17-8.08 (2H, |
| | m), 8.24 (2H, s) 8.63-8.63 (1H, br.s). |
| 54.03 | 1.77-1.67 (1H, m), 2.05-1.90 (6H, m), 2.75-2.56 (2H, m), 3.61-3.50 (2H, m), |
| | 3.74 (2H, d, J = 11.7 Hz), 4.44-4.40 (2H, m), 6.72-6.67 (2H, m), 6.80 (1H, d, J = 5.9 |
| | Hz), 7.28-7.13 (6H, m), 7.34 (1H, dd, J = 1.4, 8.7 Hz), 7.51 (1H, s), 7.76 (1H, d, J = |
| | 5.8 Hz), 8.13-8.08 (2H, m), 8.22 (1H, t, J = 5.9 Hz). |
| 55.01 | 0.73 (3H, t, J = 7.5 Hz), 1.32-1.40 (2H, m), 1.42-1.49 (2H, m), 2.54-2.66 (2H, m), |
| | 2.68-2.82 (1H, m), 2.95-3.00 (1H, m), 4.05-4.11 (1H, m), 4.33-4.50 (2H, m), |
| | 6.81 (2H, d, J = 5.6 Hz), 7.12 (1H, br.s), 7.31-7.38 (2H, m), 7.48 (1H, s), 7.63 (1H, d, |
| | J = 9.1 Hz), 7.75 (1H, d, J = 5.8 Hz), 8.13 (1H, t, J = 8.6 Hz), 8.67 (1H, t, J = 5.8 Hz). |
| 65.01 | 0.99-0.92 (6H, m), 1.35-1.28 (3H, m), 1.63-1.54 (1H, m), 1.82-1.71 (1H, m), |
| | 2.75-2.57 (2H, m), 2.86-2.76 (3H, m), 3.61-3.55 (1H, m), 4.49-4.31 (3H, m), |
| | 5.04-4.95 (1H, m), 6.85-6.76 (3H, m), 7.34-7.06 (6H, m), 7.55-7.45 (1H, m), |
| | 7.77 (1H, d, J = 5.8 Hz), 8.12 (1H, d, J = 8.4 Hz), 8.20 (2H, s), 8.35 (1H, t, J = 6.0 Hz). |
| 65.02 | 1.38 (3H, d, J = 7.2 Hz), 2.09-1.85 (2H, m), 2.77-2.61 (2H, m), 2.89-2.81 (3H, m), |
| | 4.46-4.36 (1H, m), 4.55-4.47 (2H, m), 4.95-4.64 (1H, m), 7.03-6.51 (1H, m), |
| | 7.16-7.06 (1H, m), 7.27-7.16 (3H, m), 7.36-7.28 (1H, m), 7.76-7.63 (3H, m), |
| | 8.26 (3H, s), 8.52-8.42 (1H, m), 8.72-8.52 (1H, m), 8.99-8.89 (1H, m). |

### Biological Methods

### Determination of the % Inhibition for FXIIa

Factor XIIa inhibitory activity in vitro was determined using standard published methods (see e.g. Shori et al., Biochem. Pharmacol., 1992, 43, 1209; Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC50 value for the test compound was determined.

Data acquired from this assay are shown in Table 25 using the following scale:

| Category | IC₅₀ (nM) |
|---|---|
| A | <30 |
| B | 30-100 |
| C | 100-279 |
| D | 280-1,000 |
| E | 1,000-3,000 |
| F | 3,000-10,000 |
| G | 10,000-40,000 |

TABLE 25
Human FXIIa data, molecular weight and LCMS data

| Example | Human FXIIa | Molecular | LCMS |
|---|---|---|---|
| number | IC50 (nM) | weight | Mass Ion |
| 0.01 | F | 477.2 | 478.3 |
| 0.02 | C | 509.3 | 510.4 |
| 0.03 | G | 464.2 | 465.4 |
| 0.04 | G | 545.2 | 546.5 |
| 0.05 | F | 526.2 | 527.4 |
| 0.06 | G | 479.2 | 480.4 |
| 0.07 | G | 492.2 | 493.1 |
| 0.08 | F | 558.3 | 559.2 |
| 0.09 | F | 526.2 | 527.1 |
| 0.10 | G | 527.2 | 528.2 |
| 0.11 | G | 486.2 | 487.1 |
| 0.12 | F | 491.2 | 492.1 |
| 0.13 | F | 477.2 | 478.1 |
| 0.14 | F | 464.2 | 465.1 |
| 0.15 | F | 462.2 | 463.1 |
| 0.16 | F | 462.2 | 463.1 |
| 0.17 | G | 464.2 | 465.1 |
| 0.18 | G | 462.2 | 463.1 |
| 0.19 | G | 545.3 | 545.8 |
| 0.20 | F | 462.2 | 463.1 |
| 0.21 | F | 465.2 | 466.1 |
| 0.22 | F | 476.2 | 477.1 |
| 0.23 | G | 461.2 | 462.1 |
| 0.24 | G | 532.2 | 533.2 |
| 0.25 | F | 485.2 | 486.1 |
| 0.26 | G | 526.2 | 527.1 |
| 0.27 | G | 538.2 | 539.2 |
| 0.28 | G | 511.2 | 512.1 |
| 0.29 | F | 450.2 | 451.1 |
| 0.30 | G | 451.1 | 452.1 |
| 0.31 | G | 512.2 | 513.1 |
| 0.32 | F | 481.2 | 482.1 |
| 0.33 | F | 467.1 | 468.1 |
| 0.34 | G | 460.2 | 461.2 |
| 0.35 | G | 461.2 | 462.1 |
| 0.36 | F | 529.2 | 530.2 |
| 0.37 | E | 536.1 | 537.1 |
| 0.38 | G | 543.2 | 544.2 |
| 0.39 | G | 504.2 | 505.2 |
| 0.40 | G | 517.1 | 518.1 |
| 0.41 | F | 552.2 | 553.2 |
| 0.42 | G | 491.2 | 492.2 |
| 0.43 | F | 481.2 | 482.2 |
| 0.44 | F | 480.2 | 481.1 |
| 0.45 | G | 546.2 | 547.2 |
| 0.46 | G | 488.2 | 489.1 |
| 0.47 | F | 538.1 | 539.2 |
| 0.48 | G | 500.2 | 501.1 |
| 0.49 | G | 496.2 | 497.1 |
| 0.50 | F | 475.2 | 476.1 |
| 0.51 | G | 556.2 | 557.2 |
| 0.52 | F | 491.2 | 492.1 |
| 0.53 | G | 514.2 | 515.2 |
| 0.54 | F | 492.2 | 493.2 |
| 0.55 | G | 492.2 | 493.2 |
| 0.56 | G | 482.2 | 483.2 |
| 0.57 | E | 477.2 | 478.2 |
| 0.58 | F | 508.2 | 509.1 |
| 0.59 | F | 491.2 | 492.1 |
| 0.60 | F | 553.2 | 554.2 |
| 0.61 | G | 490.2 | 491.1 |
| 0.62 | F | 477.2 | 478.1 |
| 0.63 | >12,650 | 543.2 | 544.2 |
| 0.64 | D | 544.2 | 545.4 |
| 0.65 | G | 450.2 | 451.1 |
| 0.66 | F | 434.1 | 435.1 |
| 0.67 | E | 542.2 | 543.2 |
| 0.68 | G | 540.2 | 541.2 |
| 0.69 | G | 464.2 | 465.1 |
| 0.70 | G | 506.2 | 507.2 |
| 0.71 | F | 462.2 | 463.1 |
| 0.72 | G | 492.2 | 493.1 |
| 0.73 | G | 496.2 | 497.1 |
| 0.74 | G | 528.2 | 529.1 |
| 0.75 | F | 493.2 | 494.1 |
| 0.76 | G | 529.2 | 530.2 |
| 0.77 | G | 466.1 | 467.1 |
| 0.78 | F | 450.2 | 451.1 |
| 0.79 | F | 506.2 | 507.2 |
| 0.80 | G | 511.2 | 512.2 |
| 0.81 | F | 543.2 | 544.2 |
| 0.82 | G | 466.1 | 467.1 |
| 0.83 | >12,650 | 540.2 | 541.2 |
| 0.84 | G | 491.2 | 492.1 |
| 0.85 | G | 499.2 | 500.2 |
| 0.86 | E | 469.2 | 470.2 |
| 0.87 | D | 504.2 | 505.4 |
| 0.88 | G | 520.2 | 521.5 |
| 0.89 | G | 504.2 | 505.5 |
| 0.90 | G | 506.2 | 507.5 |
| 0.91 | G | 509.2 | 510.5 |
| 0.92 | F | 504.2 | 505.5 |
| 0.93 | E | 502.2 | 503.5 |
| 0.94 | E | 519.2 | 520.4 |
| 0.95 | G | 519.2 | 520.4 |
| 0.96 | F | 519.2 | 520.4 |
| 0.97 | E | 504.2 | 505.2 |
| 0.98 | G | 504.2 | 505.2 |
| 0.99 | G | 504.2 | 505.5 |
| 3.01 | C | 509.3 | 510.1 |
| 3.02 | C | 523.3 | 524.2 |
| 3.03 | E | 441.2 | 441.9 |
| 3.04 | C | 491.3 | 492.1 |
| 3.05 | E | 507.2 | 508.0 |
| 3.06 | E | 481.2 | 481.8 |
| 3.07 | G | 483.2 | 484.1 |
| 3.08 | D | 469.2 | 470.4 |
| 3.09 | D | 497.3 | 497.8 |
| 3.10 | B | 529.3 | 530.4 |
| 3.11 | C | 473.3 | 474.4 |
| 3.12 | F | 507.2 | 508.4 |
| 3.13 | C | 507.2 | 508.4 |
| 3.14 | C | 491.3 | 492.1 |
| 3.15 | E | 487.3 | 488.4 |
| 3.16 | F | 479.3 | 480.4 |
| 3.17 | E | 509.3 | 510.4 |
| 3.18 | D | 495.2 | 495.1 |
| 3.19 | D | 495.2 | 495.1 |
| 3.20 | D | 495.2 | 495.1 |
| 3.21 | C | 512.3 | 513.4 |
| 3.22 | F | 411.3 | 412.3 |
| 3.23 | G | 475.2 | 476.1 |
| 3.24 | F | 552.2 | 553.1 |
| 3.25 | G | 499.2 | 500.2 |
| 3.26 | G | 480.1 | 481.1 |
| 3.27 | G | 531.2 | 532.3 |
| 3.28 | G | 456.2 | 457.1 |
| 3.29 | G | 573.2 | 574.2 |
| 3.30 | G | 470.2 | 471.2 |
| 3.31 | F | 492.2 | 493.2 |
| 3.32 | G | 542.2 | 543.2 |
| 3.33 | G | 545.2 | 546.2 |
| 3.34 | G | 475.2 | 476.1 |
| 3.35 | G | 468.2 | 469.2 |
| 3.36 | G | 468.2 | 469.2 |
| 3.37 | G | 481.2 | 482.2 |
| 3.38 | E | 495.2 | 496.2 |
| 3.39 | G | 495.2 | 496.2 |
| 3.40 | G | 496.2 | 497.2 |
| 3.41 | E | 467.2 | 468.2 |
| 3.42 | B | 509.3 | 510.4 |
| 3.101 | E | 517.2 | 518.0 |
| 3.102 | G | 459.2 | 460.1 |
| 3.103 | G | 483.2 | 484.2 |
| 3.104 | G | 498.3 | 499.6 |
| 3.105 | G | 499.2 | 500.5 |
| 5.101 | F | 497.3 | 497.8 |
| 5.102 | D | 483.2 | 484.2 |
| 5.103 | E | 481.2 | 481.9 |
| 5.104 | G | 455.2 | 456.3 |
| 5.105 | D | 523.3 | 524.1 |
| 5.106 | F | 523.3 | 524.1 |
| 6.01 | G | 491.3 | 492.4 |
| 6.02 | D | 391.2 | 392.3 |
| 6.03 | G | 519.3 | 520.4 |
| 6.04 | G | 519.3 | 520.4 |
| 6.05 | E | 419.2 | 420.3 |
| 6.06 | D | 419.2 | 420.3 |
| 6.07 | D | 433.2 | 434.3 |
| 6.08 | F | 495.3 | 496.4 |
| 6.09 | C | 405.2 | 406.3 |
| 6.10 | C | 419.2 | 420.4 |
| 6.11 | D | 447.3 | 448.4 |
| 6.12 | G | 406.2 | 407.4 |
| 6.13 | D | 406.2 | 407.4 |
| 6.14 | E | 440.3 | 441.4 |
| 6.15 | F | 406.2 | 407.3 |
| 6.16 | G | 421.2 | 422.3 |
| 6.17 | G | 407.2 | 408.3 |
| 6.18 | F | 419.2 | 420.3 |
| 6.19 | B | 487.3 | 488.5 |
| 6.20 | D | 483.2 | 484.4 |
| 6.21 | G | 405.2 | 406.3 |
| 6.22 | G | 435.2 | 436.3 |
| 6.23 | C | 459.3 | 460.4 |
| 6.24 | G | 406.2 | 407.1 |
| 6.25 | F | 505.3 | 506.1 |
| 6.26 | G | 509.2 | 510.1 |
| 9.01 | D | 413.2 | 414.3 |
| 9.02 | D | 381.2 | 382.4 |
| 9.03 | E | 455.2 | 456.3 |
| 9.04 | F | 412.2 | 413.3 |
| 9.05 | G | 521.2 | 522.5 |
| 9.06 | C | 419.2 | 420.4 |
| 9.07 | C | 411.2 | 412.4 |
| 9.08 | E | 454.2 | 454.9 |
| 9.09 | F | 397.2 | 398.2 |
| 9.10 | F | 410.3 | 411.2 |
| 9.11 | G | 445.2 | 446.1 |
| 9.12 | G | 411.3 | 412.2 |
| 9.16 | G | 490.1 | 491.0 |
| 9.17 | F | 496.2 | 497.1 |
| 9.18 | D | 482.2 | 483.2 |
| 9.19 | G | 568.3 | 569.2 |
| 9.20 | G | 472.2 | 473.1 |
| 9.21 | G | 491.1 | 492.0 |
| 9.22 | G | 497.2 | 498.2 |
| 9.23 | G | 471.2 | 472.1 |
| 9.24 | C | 437.2 | 438.1 |
| 9.25 | E | 502.2 | 503.1 |
| 9.26 | F | 483.2 | 484.1 |
| 9.27 | B | 453.2 | 454.1 |
| 10.01 | B | 501.3 | 502.4 |
| 10.02 | D | 425.2 | 426.1 |
| 10.03 | D | 462.2 | 463.1 |
| 10.04 | D | 482.2 | 483.1 |
| 10.05 | D | 504.3 | 505.1 |
| 10.06 | G | 392.2 | 393.0 |
| 10.07 | D | 613.3 | 614.1 |
| 10.08 | F | 538.3 | 539.1 |
| 10.09 | E | 507.2 | 508.1 |
| 10.10 | E | 493.2 | 494.0 |
| 10.11 | D | 535.3 | 536.1 |
| 10.12 | E | 495.2 | 496.0 |
| 10.13 | F | 442.2 | 443.0 |
| 10.14 | G | 493.2 | 492.2 |
| 10.15 | F | 495.2 | 496.1 |
| 10.16 | G | 444.2 | 445.3 |
| 10.17 | G | 468.2 | 469.1 |
| 10.18 | G | 491.2 | 491.9 |
| 10.19 | F | 461.2 | 462.1 |
| 10.20 | D | 503.3 | 504.2 |
| 12.01 | B | 552.3 | 553.4 |
| 12.02 | D | 482.3 | 483.4 |
| 12.03 | A | 594.3 | 595.5 |
| 12.04 | D | 580.3 | 581.3 |
| 12.05 | B | 597.4 | 598.4 |
| 12.06 | A | 608.4 | 609.3 |
| 12.07 | A | 614.3 | 615.4 |
| 12.08 | E | 608.4 | 609.4 |
| 12.09 | E | 588.4 | 589.5 |
| 12.10 | F | 634.4 | 635.5 |
| 12.11 | D | 537.3 | 538.4 |
| 12.12 | F | 559.3 | 560.2 |
| 17.01 | F | 531.3 | 532.5 |
| 17.02 | D | 431.2 | 432.3 |
| 17.03 | E | 445.2 | 446.3 |
| 17.04 | F | 445.2 | 446.3 |
| 17.05 | D | 417.2 | 418.3 |
| 17.06 | F | 471.3 | 472.4 |
| 17.07 | G | 445.2 | 446.4 |
| 17.08 | D | 431.2 | 432.3 |
| 17.09 | C | 459.3 | 460.5 |
| 17.10 | D | 494.3 | 495.2 |
| 17.11 | D | 480.3 | 481.2 |
| 18.101 | E | 483.3 | 484.2 |
| 18.102 | E | 545.2 | 546.4 |
| 18.103 | G | 371.2 | 372.2 |
| 18.104 | E | 419.2 | 420.3 |
| 18.105 | G | 448.2 | 449.3 |
| 18.106 | G | 419.2 | 420.3 |
| 18.107 | G | 385.2 | 386.3 |
| 18.108 | G | 411.2 | 412.3 |
| 18.109 | G | 460.3 | 461.4 |
| 20.01 | G | 534.2 | 535.2 |
| 20.02 | G | 491.2 | 492.1 |
| 20.03 | G | 492.2 | 493.2 |
| 20.04 | G | 456.2 | 457.1 |
| 20.05 | G | 528.2 | 529.2 |
| 20.06 | G | 542.2 | 543.2 |
| 20.07 | G | 479.2 | 480.1 |
| 20.08 | G | 520.2 | 521.2 |
| 20.09 | G | 549.2 | 550.1 |
| 20.10 | G | 532.2 | 533.2 |
| 24.01 | G | 376.1 | 376.9 |
| 24.02 | G | 338.2 | 338.9 |
| 24.03 | G | 334.2 | 335.3 |
| 27.01 | D | 481.2 | 482.3 |
| 27.02 | C | 516.3 | 517.4/519.4 |
| 27.03 | A | 601.3 | 602.4 |
| 27.04 | B | 602.3 | 603.5 |
| 27.05 | A | 642.3 | 643.5 |
| 27.06 | B | 560.2 | 561.1 |
| 27.07 | F | 534.3 | 535.4 |
| 27.08 | D | 467.2 | 468.1 |
| 32.01 | G | 439.2 | 440.0 |
| 32.02 | G | 423.3 | 424.2 |
| 32.03 | E | 437.3 | 438.2 |
| 32.04 | G | 471.3 | 472.4 |
| 32.06 | G | 423.3 | 424.4 |
| 32.07 | G | 423.3 | 424.3 |
| 32.08 | E | 437.3 | 438.4 |
| 32.09 | G | 485.3 | 485.4 |
| 32.10 | G | 423.3 | 424.4 |
| 32.11 | F | 409.2 | 410.3 |
| 32.12 | G | 409.2 | 410.4 |
| 32.13 | G | 463.3 | 464.4 |
| 32.14 | G | 449.3 | 450.4 |
| 32.15 | G | 437.3 | 438.4 |
| 34.01 | G | 507.2 | 508.1 |
| 34.02 | F | 513.3 | 514.2 |
| 34.03 | F | 482.2 | 483.2 |
| 34.04 | G | 459.2 | 460.1 |
| 34.05 | G | 482.2 | 483.2 |
| 34.06 | F | 483.2 | 484.2 |
| 34.07 | G | 483.2 | 484.2 |
| 34.08 | G | 497.3 | 498.2 |
| 39.01 | G | 397.2 | 397.8 |
| 39.02 | F | 397.2 | 398.2 |
| 39.03 | G | 383.2 | 384.3 |
| 39.04 | G | 487.3 | 488.4 |
| 39.05 | F | 517.3 | 518.5 |
| 39.06 | G | 427.3 | 428.3 |
| 39.07 | G | 473.3 | 474.4 |
| 39.08 | G | 411.3 | 412.4 |
| 39.09 | F | 409.2 | 410.3 |
| 39.10 | F | 423.3 | 424.4 |
| 39.11 | G | 453.3 | 454.4 |
| 43.01 | F | 388.2 | 389.3 |
| 43.02 | D | 499.3 | 500.4 |
| 43.03 | D | 507.3 | 508.4 |
| 43.04 | G | 432.2 | 433.3 |
| 43.05 | G | 443.3 | 444.5 |
| 43.06 | E | 443.2 | 444.4 |
| 43.07 | F | 445.2 | 446.4 |
| 44.01 | D | 495.2 | 496.1 |
| 44.02 | G | 499.3 | 500.2 |
| 44.03 | G | 468.2 | 469.2 |
| 44.04 | G | 469.2 | 470.2 |
| 44.05 | G | 469.2 | 470.2 |
| 44.06 | G | 483.2 | 484.2 |
| 45.01 | D | 417.2 | 418.3 |
| 45.02 | F | 415.2 | 416.3 |
| 45.03 | E | 417.2 | 418.3 |
| 45.04 | G | 403.2 | 404.3 |
| 45.05 | G | 389.2 | 390.3 |
| 54.01 | D | 417.2 | 418.3 |
| 54.02 | E | 431.2 | 432.3 |
| 54.03 | F | 461.2 | 462.4 |
| 55.01 | G | 462.2 | 463.3 |
| 55.02 | G | 434.1 | 435.1 |
| 55.03 | G | 471.2 | 472.1 |
| 55.04 | G | 535.1 | 536.1 |
| 55.05 | E | 581.2 | 582.2 |
| 65.01 | D | 461.3 | 462.5 |
| 65.02 | E | 419.2 | 420.4 |
| 71.01 | C | 443.2 | 444.2 |
| 71.02 | E | 451.2 | 452.1 |
| 71.03 | E | 475.3 | 476.3 |
| 71.04 | E | 461.3 | 462.3 |
| 71.05 | F | 507.3 | 508.3 |

### Determination of the % Inhibition for FXIa

FXIa inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human FXIa (Enzyme Research Laboratories) was incubated at 25° C. with the fluorogenic substrate Z-Gly-Pro-Arg-AFC and various concentrations of the test compound. Residual enzyme activity (initial rate of reaction) was determined by measuring the change in fluorescence at 410 nm and the IC50 value for the test compound was determined.

Data acquired from this assay are shown in Table 26 using the following scale:

| Category | IC₅₀ (nM) |
|---|---|
| A | <30 |
| B | 30-100 |
| C | 100-300 |
| D | 300-1,000 |
| E | 1,000-3,000 |
| F | 3,000-10,000 |
| G | 10,000-40,000 |
| H | >40,000 |

TABLE 26
Selectivity; FXIa data

| Example number | Human FXIa IC50 (nM) |
|---|---|
| 0.64 | H |
| 0.94 | H |
| 0.95 | H |
| 0.96 | H |
| 0.97 | H |
| 0.98 | H |
| 0.99 | H |
| 3.01 | H |
| 3.42 | H |
| 6.09 | G |
| 6.10 | G |
| 6.11 | F |
| 6.12 | H |
| 6.13 | H |
| 6.14 | G |
| 6.15 | H |
| 6.16 | G |
| 6.17 | G |
| 6.18 | F |
| 6.19 | D |
| 6.20 | D |
| 6.21 | H |
| 6.22 | F |
| 6.23 | H |
| 6.24 | G |
| 6.25 | G |
| 9.01 | G |
| 9.02 | H |
| 9.03 | H |
| 9.04 | H |
| 9.05 | E |
| 9.06 | G |
| 9.07 | H |
| 9.08 | G |
| 9.24 | H |
| 9.27 | H |
| 10.01 | G |
| 10.02 | H |
| 10.03 | H |
| 10.04 | H |
| 10.05 | H |
| 10.06 | H |
| 10.07 | H |
| 10.08 | H |
| 10.09 | H |
| 10.10 | H |
| 10.11 | G |
| 10.12 | H |
| 10.13 | H |
| 10.14 | H |
| 10.15 | H |
| 10.16 | H |
| 10.17 | G |
| 10.18 | G |
| 12.01 | H |
| 12.02 | G |
| 12.03 | H |
| 12.04 | G |
| 12.05 | F |
| 12.06 | F |
| 12.07 | E |
| 12.08 | G |
| 12.09 | H |
| 12.10 | H |
| 12.11 | H |
| 17.05 | G |
| 17.06 | E |
| 17.07 | F |
| 17.08 | H |
| 17.09 | G |
| 17.10 | H |
| 17.11 | H |
| 18.105 | G |
| 18.106 | H |
| 18.107 | H |
| 18.108 | H |
| 18.109 | H |
| 27.01 | H |
| 27.02 | F |
| 27.03 | G |
| 27.04 | H |
| 27.05 | G |
| 27.06 | H |
| 27.07 | H |
| 27.08 | H |
| 32.10 | H |
| 32.11 | G |
| 32.12 | H |
| 32.13 | H |
| 32.14 | H |
| 32.15 | H |
| 39.09 | H |
| 39.10 | G |
| 39.11 | H |
| 43.01 | H |
| 43.02 | G |
| 43.03 | G |
| 43.04 | G |
| 43.05 | H |
| 43.06 | G |
| 43.07 | G |
| 45.01 | F |
| 45.02 | F |
| 45.03 | F |
| 45.04 | F |
| 45.05 | H |
| 54.01 | H |
| 54.02 | G |
| 54.03 | H |
| 65.01 | F |
| 65.02 | H |
| 71.01 | >10,000 |
| 71.02 | >10,000 |
| 71.03 | E |
| 71.04 | F |
| 71.05 | D |

### Numbered Embodiments

1. A compound of formula (I),

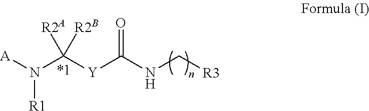

wherein
*1 denotes a chiral centre
n=0, 1 or 2;
A is selected from H, —(C═O)R4, —SO₂R6, and —(CH₂)—R13;
Y is either a bond, or —[CHR5]-;
R1 is H or alkyl^{b};
R2^{A} is selected from H, alkyl, —(CH₂)₀₋₃aryl, —(CH₂)₀₋₃heteroaryl, —(CH₂)₀₋₃cycloalkyl, —(CH₂)₀₋₃-[benzothiophene], —(CH₂)₀₋₃-[indole], and

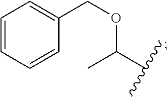 [SMILES: *C(C)OCc1ccccc1]

or, when Y is a bond, R1 and R2^{A}, together with nitrogen atom to which R1 is attached and the carbon atom to which R2^{A} is attached, may be linked by alkylene to form a 4-, 5-, or 6-membered saturated heterocycle, optionally wherein the 4-, 5-, or 6-membered saturated heterocycle may be substituted with aryl, or wherein two adjacent carbon atoms on the 4-, 5-, or 6-membered saturated heterocycle may be linked to form a 6-membered aromatic ring, or wherein two adjacent carbon atoms on the 4-, 5-, or 6-membered saturated heterocycle may be linked to form a 3-, 4-, or 5-membered saturated hydrocarbon ring which may be optionally mono- or di-substituted by alkyl^{b};
when Y is —[CHR5]-, R5 is H; or,
when Y is —[CHR5]-, together with the carbon atoms to which each of R5 and R2^{A} are attached, R5 and R2^{A} may be linked by alkylene to form a 4-, 5-, 6-membered saturated ring; or,
when Y is —[CHR5]-, together with the nitrogen atom to which R1 is attached, the carbon atom to which R5 is attached, and the carbon atom to which R2^{A} and R2^{B} are both attached, R5 and R1 may be linked by alkylene to form a saturated 4-, 5-, or 6-membered heterocycle, optionally wherein one atom on the saturated 4-, 5-, or 6-membered heterocycle may be linked by alkylene to join with R2^{A};
R2^{B} is H or alkyl^{b}; or,
R2^{A} and R2^{B}, together with the carbon to which R2^{A} and R2^{B} are both attached, may be linked by alkylene or heteroalkylene to form a 3-, 4-, 5-, or 6-membered saturated ring, optionally wherein the 3-, 4-, 5-, or 6-membered saturated ring contains one or two ring members that are selected from N and O;
R3 is:
(i) a fused 6,5- or 6,6-bicyclic ring, containing one heteroatom selected from S and N, wherein at least one of the rings is aromatic and, optionally the bicyclic ring contains one additional heteroatom independently selected from N, O and S;
   optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring; or
(ii) phenyl, pyridyl, or thiophenyl, which may be optionally substituted with 1, 2 or 3 substituents independently selected from alkyl^{b}, alkoxy, OH, NH₂ halo, CN, CF₃, —C(═NH)NH₂, and heteroaryl^{b};
   wherein when n=1, and R3 is phenyl substituted with at least one —(CH₂NH₂), R2^{A} is alkyl and R2^{B} is H; or
(iii)

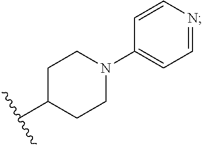 [SMILES: *C1CCN(c2ccncc2)CC1]

R4 is one of:
(i) a group of formula (II),

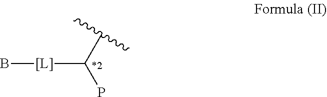

wherein -[L]- is a bond, —[(CH₂)₁₋₄]—, —[(CH₂)—O—(CH₂)]—, or —[O—(CH₂)]—; and P is alkoxy, OH or NR11R12;
wherein *2 denotes a chiral centre, and
wherein when -[L]- is a bond, B is a C₁₋₄ linear or branched chain hydrocarbon, and
wherein when -[L]- is —[(CH₂)₁₋₄]—, —[(CH₂)—O—(CH₂)]—, or —[O—(CH₂)]—, B is OH, aryl, heteroaryl, heterocyclyl, cycloalkyl or N or,

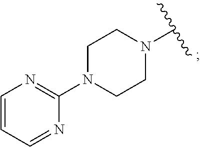 [SMILES: *N1CCN(c2ncccn2)CC1]

(ii) —(CH₂)ₘ-[fused 6,5- or 6,6-heteroaromatic bicyclic ring], wherein at least one ring atom is a heteroatom selected from O, N or S, and optionally, 1, 2 or 3 additional ring atoms may be selected from N or NH; wherein the fused 6,5- or 6,6-heteroaromatic bicyclic ring may be optionally substituted with 1, 2 or 3 substituents independently selected from alkyl^{b}; wherein the 6,5-heteroaromatic bicyclic ring may be attached to —(CH₂)ₘ— via the 6- or 5-membered ring; or, (iii) methyl, —C(CH₃)₂(OH), —C(CH₃)₂(NHMe), —(CH₂)ₘ-(aryl), —(CH₂)ₘ-(cycloalkyl), —(CH₂)ₘ-(heteroaryl), —(CH₂)ₘ-(heterocyclyl), —(CH₂)-(alkyl), —(CH(halo)₂), —(CH₂)ₘ—(NR8R9), —(CH₂)ₘ—(NR10R7), —(CH₂)ₘ—O—(CH₂)ₖ-(aryl), —(CH₂)ₘ—(SO₂)—(CH₂)ₖ-(aryl),
   —(CH₂)ₘ-(alkoxy), —(CH₂)ₘ—O—(CH₂)ₖ-(heteroaryl), or —(CH₂)ₘ-[pyridone, which may be optionally substituted by alkyl^{b}, or CF₃];
wherein k=0, 1, 2, or 3;
wherein m=0, 1, 2 or 3;
wherein:
   when Y is —[CHR5]- and R5 is H, R2^{A} is CH₂-aryl or H; and
   when Y is —[CHR5]-, R3 is;

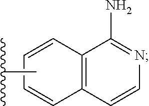

when A is H, R3 is

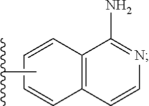

and
when R3 is

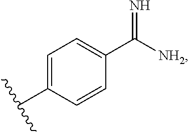 [SMILES: *c1ccc(C(=N)N)cc1]

R2^{A} is not H;
wherein:
R6 is alkyl or —(CH₂)₀₋₃-(aryl);
R7 is independently selected from H, —SO₂CH₃, methyl, ethyl, propyl, isopropyl, and cycloalkyl;
R8 and R9 are independently selected from H, —SO₂CH₃, alkyl^{b}, heteroaryl^{b}, and cycloalkyl; or R8 and R9 together with the nitrogen atom to which they are attached form a carbon-containing 4-, 5-, 6- or 7-membered heterocyclic ring, optionally containing an additional heteroatom selected from N, NR10, S, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃; or R8 and R9 together with the nitrogen atom to which they are attached form a carbon-containing 5- or 6-membered heterocyclic ring, which is fused to an aryl^{b} or a heteroaryl^{b};
R10 is independently selected from H, —SO₂R6, alkyl^{b}, —(CH₂)₀₋₃aryl^{b}, —(CH₂)₀₋₃heteroaryl^{b}, cycloalkyl, —(C═O)-(aryl), and —(CH₂)₀₋₃heterocyclyl^{b}; or R10 is a carbon-containing 4-, 5-, 6- or 7-membered heterocyclic ring, optionally containing an additional heteroatom selected from N, NR7, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃;
R11 and R12 are independently selected from H, alkyl^{b}, —SO₂R6, cycloalkyl, —(C═O)O-(alkyl^{b}), —(C═O)-phenyl, —CH₂-phenyl, and CH₂—COOH; or R11 and R12 together with the nitrogen atom to which they are attached form a carbon-containing 4-, 5-, 6- or 7-membered heterocyclic ring optionally containing an additional heteroatom selected from N, O, and NR10, wherein the heterocyclic ring may be optionally mono- or di-substituted with substituents independently selected from alkyl^{b}, OH, halo and CF₃;
R13 is selected from heteroaryl, cycloalkyl, heterocyclyl and aryl^{b};
wherein:
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R7)₂ and fluoro;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₁-C₆)alkoxy, OH, —NR8R9, —NHCOCH₃, —CO(heterocyclyl^{b}), —COOR8, —CONR8R9, CN, CF₃, halo, oxo and heterocyclyl^{b};
alkyl^{b} is a linear saturated hydrocarbon having up to 6 carbon atoms (C₁-C₆) or a branched saturated hydrocarbon of between 3 and 6 carbon atoms (C₃-C₆); alkyl may optionally be substituted with 1 or 2 substituents independently selected from (C₃-C₆)alkoxy, OH, —N(R7)₂, —NHCOCH₃, CF₃, halo, oxo and cyclopropane;
alkylene is a bivalent linear saturated hydrocarbon having 1 to 5 carbon atoms (C₁-C₅); alkylene may optionally be substituted with 1 or 2 substituents independently selected from alkyl, (C₁-C₆)alkoxy, OH, CN, CF₃ and halo;
aryl is phenyl, biphenyl or naphthyl; aryl may be optionally substituted with 1, 2 or 3 substituents independently selected from alkyl, alkoxy, OH, —SO₂CH₃, halo, —SO₂NR8R9, CN, —(CH₂)₀₋₃—O-heteroaryl^{b}, aryl^{b}, —O-aryl^{b}, —(CH₂)₀₋₃-heterocyclyl^{b},
—(CH₂)₁₋₃-aryl^{b}, —(CH₂)₀₋₃-heteroaryl^{b}, —COOR8, —CONR8R9, —(CH₂)₀₋₃—NR8R9, OCF₃ and CF₃; or two adjacent carbon ring atoms on the aryl may be optionally linked by a heteroalkylene to form a non-aromatic ring containing 5, 6, or 7 ring members which may be optionally substituted with OH; or optionally wherein two adjacent ring atoms on aryl are linked to form a 5- or 6-membered aromatic ring containing 1 or 2 heteroatoms that are selected from N, NR10, S, and O;
aryl^{b} is phenyl, biphenyl or naphthyl, which may be optionally substituted with 1, 2 or 3 substituents independently selected from methyl, ethyl, propyl, isopropyl, alkoxy, OH, —SO₂CH₃, N(R7)₂, halo, CN, and CF₃; or two adjacent carbon ring atoms on the aryl may be optionally linked by a heteroalkylene to form a non-aromatic ring containing 5, 6, or 7 ring members;
cycloalkyl is monocyclic saturated hydrocarbon ring of between 3 and 6 carbon atoms (C3-C₆); cycloalkyl may optionally be substituted with 1 or 2 substituents independently selected from methyl, ethyl, propyl, isopropyl, methoxy, ethoxy, propoxy, isopropoxy, OH, CN, CF₃ and halo; optionally wherein two adjacent ring atoms on cycloalkyl are linked to form a 5- or 6-membered saturated hydrocarbon ring;
halo is F, Cl, Br, or I;
heteroalkylene is a bivalent linear saturated hydrocarbon having 2 to 5 carbon atoms (C₂-C₅), wherein 1 or 2 of the 2 to 5 carbon atoms are replaced with NR10, S, or O; heteroalkylene may optionally be substituted with 1 or 2 substituents independently selected from alkyl, (C₁-C₆)alkoxy, OH, CN, CF₃ and halo;
heteroaryl is a 5- or 6-membered carbon-containing aromatic ring containing one, two or three ring members that are selected from N, NR10, S, and O; heteroaryl may be optionally substituted with 1, 2 or 3 substituents independently selected from alkyl, alkoxy, heteroaryl^{b}, phenyl, cycloalkyl, OH, OCF₃, halo, heterocyclyl^{b}, CN, and CF₃;
heteroaryl^{b} is a 5- or 6-membered carbon-containing aromatic ring containing one, two or three ring members that are selected from N, NR10, S, and O; heteroaryl^{b} may be optionally substituted with 1, 2 or 3 substituents independently selected from methyl, ethyl, propyl, isopropyl, alkoxy, OH, OCF₃, COOCH₃, COOCH₂CH₃, COO—(CH₂)₂—CH₃, COO-(iPr), halo, CN, and CF₃;
heterocyclyl is a 4-, 5-, 6-, or 7-membered carbon-containing non-aromatic ring containing one, two, three, or four ring members that are selected from N, NR10, S, SO, SO₂ and O; heterocyclyl may be optionally substituted with 1, 2, 3, or 4 substituents independently selected from alkyl, alkoxy, aryl^{b}, OH, OCF₃, halo, oxo, CN, NR8R9, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two ring atoms on heterocyclyl are linked with an heteroalkylene to form a non-aromatic ring containing 5, 6, or 7 ring members; or optionally wherein two adjacent ring atoms on heterocyclyl are linked to form a 5- or 6-membered aromatic ring which may optionally contain 1 or 2 heteroatoms that are selected from N, NR10, S, and O;
heterocyclyl^{b} is a 4-, 5-, 6-, or 7-membered carbon-containing non-aromatic ring containing one, two or three ring members that are selected from N, NR7, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃;
and tautomers, isomers, stereoisomers (including enantiomers, diastereoisomers and racemic and scalemic mixtures thereof), deuterated isotopes, and pharmaceutically acceptable salts and/or solvates thereof.
2. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein n=0.
3. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein n=1.
4. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein n=2.
5. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein A is H.
6. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein A is —(C═O)R4.
7. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is a group of formula (II),

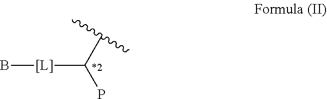

wherein -[L]- is a bond; P is alkoxy, OH or NR11R12;
and B is a C₁₋₄ linear or branched chain hydrocarbon.
8. A compound of formula (I) according to numbered embodiment 7, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is methyl.
9. A compound of formula (I) according to numbered embodiment 7, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is sec-butyl.
10. A compound of formula (I) according to any one of numbered embodiments 7 to 9, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is alkoxy.
11. A compound of formula (I) according to any one of numbered embodiments 7 to 9, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is OH.
12. A compound of formula (I) according to any one of numbered embodiments 7 to 9, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NR11R12.
13. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is N(CH₃)₂.
14. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is N(CH₃)(iPr).
15. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is pyrrolidinyl.
16. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is

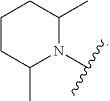 [SMILES: *N1C(C)CCCC1C]

17. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NH₂.
18. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is a group of formula (II),

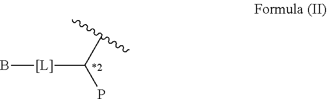

wherein -[L]- is —[(CH₂)₁₋₄]—, —[(CH₂)—O—(CH₂)]—, or —[O—(CH₂)]—
P is alkoxy, OH or NR11R12;
and B is OH, aryl, heteroaryl, heterocyclyl, cycloalkyl or

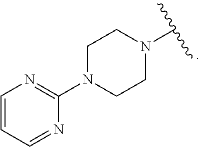 [SMILES: *N1CCN(c2ncccn2)CC1]

19. A compound of formula (I) according to numbered embodiment 18, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is alkoxy.
20. A compound of formula (I) according to numbered embodiment 18, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is OH.
21. A compound of formula (I) according to numbered embodiment 18, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NR11R12.
22. A compound of formula (I) according to numbered embodiment 21, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NHR11.
23. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof, wherein P is NH₂.
24. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NH(iPr).
25. A compound of formula (I) according to numbered embodiment 21, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is N(CH₃)₂.
26. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NH(Boc).
27. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NH(CH₃).
28. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NH(cyclohexyl).
29. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NH(SO₂CH₃).
30. A compound of formula (I) according to numbered embodiment 21, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is pyrrolidinyl.
31. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NHC(═O)(phenyl).
32. A compound of formula (I) according to numbered embodiment 21, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is piperazinyl.
33. A compound of formula (I) according to any one of numbered embodiments 21 and 22, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein P is NHCH₂COOH.
34. A compound of formula (I) according to any one of numbered embodiments 18 to 33, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[(CH₂)₁₋₄]—.
35. A compound of formula (I) according to numbered embodiment 34, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[(CH₂)]—.
36. A compound of formula (I) according to numbered embodiment 34, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[(CH₂)₂]—.
37. A compound of formula (I) according to numbered embodiment 34, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[(CH₂)₃]—.
38. A compound of formula (I) according to numbered embodiment 34, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[(CH₂)₄]—.
39. A compound of formula (I) according to any one of numbered embodiments 18 to 33, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[(CH₂)—O—(CH₂)]—.
40. A compound of formula (I) according to any one of numbered embodiments 18 to 33, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein -[L]- is —[O—(CH₂)]—.
41. A compound of formula (I) according to any one of numbered embodiments 18 to 40, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is OH.
42. A compound of formula (I) according to any one of numbered embodiments 18 to 40, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is aryl.
43. A compound of formula (I) according to numbered embodiment 42, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is phenyl.
44. A compound of formula (I) according to any one of numbered embodiments 18 to 40, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is heteroaryl.
45. A compound of formula (I) according to numbered embodiment 44, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is pyridyl.
46. A compound of formula (I) according to any one of numbered embodiments 18 to 40, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is heterocyclyl.
47. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is piperidinyl, which may be optionally substituted with up to two substituents selected from halo, optionally wherein the halo substituents are fluoro.
48. A compound of formula (I) according to numbered embodiment 47, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is piperidinyl.
49. A compound of formula (I) according to numbered embodiment 47, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is piperidinyl, which is substituted by two fluoro substituents.
50. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is pyrrolidinyl, which may be optionally substituted with up to two substituents selected from halo, optionally wherein the halo substituents are fluoro.
51. A compound of formula (I) according to numbered embodiment 50, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is pyrrolidinyl.
52. A compound of formula (I) according to numbered embodiment 50, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is pyrrolidinyl which is substituted by two fluoro substituents.
53. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is 1,2,3,4-tetrahydroisoquinolyl.
54. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is indolinyl.
55. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is isoindolinyl.
56. A compound of formula (I) according to numbered embodiment 46, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is morpholinyl.
57. A compound of formula (I) according to any one of numbered embodiments 18 to 40, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is cycloalkyl.
58. A compound of formula (I) according to numbered embodiment 57, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is cyclohexyl.
59. A compound of formula (I) according to any one of numbered embodiments 18 to 40, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein B is

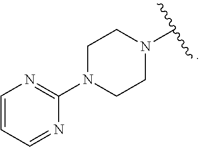 [SMILES: *N1CCN(c2ncccn2)CC1]

60. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is
   —(CH₂)ₘ-[fused 6,5- or 6,6-heteroaromatic bicyclic ring], wherein at least one ring atom is a heteroatom selected from O, N or S, and optionally, 1, 2 or 3 additional ring atoms may be selected from N or NH; wherein the fused 6,5- or 6,6-heteroaromatic bicyclic ring may be optionally substituted with 1, 2 or 3 substituents independently selected from alkyl^{b}; wherein the 6,5-heteroaromatic bicyclic ring may be attached to —(CH₂)ₘ— via the 6- or 5-membered ring.
61. A compound of formula (I) according to numbered embodiment 60, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein at least one ring atom is selected from O.
62. A compound of formula (I) according to any of numbered embodiments 60 to 61, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[benzofuranyl].
63. A compound of formula (I) according to numbered embodiment 60, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein two ring atoms are N.
64. A compound of formula (I) according to numbered embodiment 60, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein three ring atoms are N.
65. A compound of formula (I) according to numbered embodiment 60, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein four ring atoms are N.
66. A compound of formula (I) according to any of numbered embodiments 60 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the 6,5-heteroaromatic bicyclic ring is be attached to —(CH₂)ₘ— via the 6-membered ring.
67. A compound of formula (I) according to any of numbered embodiments 60 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the 6,5-heteroaromatic bicyclic ring is be attached to —(CH₂)ₘ— via the 5-membered ring.
68. A compound of formula (I) according to any of numbered embodiments 60 to 61 and 63 to 67, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the fused 6,5- or 6,6-heteroaromatic bicyclic ring is substituted with 1, 2 or 3 substituents independently selected from alkyl^{b}.
69. A compound of formula (I) according to numbered embodiment 68, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the fused 6,5- or 6,6-heteroaromatic bicyclic ring is be substituted with 1, 2 or 3 substituents independently selected from methyl and isopropyl.
70. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is methyl.
71. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —C(CH₃)₂(OH).
72. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —C(CH₃)₂(NHMe).
73. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(aryl).
74. A compound of formula (I) according to numbered embodiment 73, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(phenyl).
75. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(cycloalkyl).
76. A compound of formula (I) according to numbered embodiment 75, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[cyclopropyl, substituted by one —CF₃].
77. A compound of formula (I) according to numbered embodiment 75, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[cyclohexyl, substituted by one OH].
78. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(heteroaryl).
79. A compound of formula (I) according to numbered embodiment 78, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is

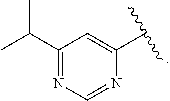 [SMILES: *c1cc(C(C)C)ncn1]

80. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(heterocyclyl).
81. A compound of formula (I) according to numbered embodiment 80, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is

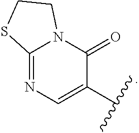 [SMILES: *c1cnc2n(c1=O)CCS2]

82. A compound of formula (I) according to numbered embodiment 80, or a tautomer, isomer, stereoisomer (including an enantiomer, a diastereoisomer and a racemic and scalemic mixture thereof), a deuterated isotope, and a pharmaceutically acceptable salt and/or solvate thereof,

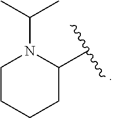 [SMILES: *C1CCCCN1C(C)C]

wherein R4 is
83. A compound of formula (I) according to numbered embodiment 80, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is pyrrolidinyl.
84. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)-(alkyl).
85. A compound of formula (I) according to numbered embodiment 84, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is

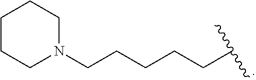 [SMILES: *CCCCCN1CCCCC1]

86. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH(halo)₂).
87. A compound of formula (I) according to numbered embodiment 86, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —CHF₂.
88. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—(NR8R9).
90. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is

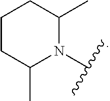 [SMILES: *N1C(C)CCCC1C]

90. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is —N(CH₃)₂.
91. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is piperidinyl.
92. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is pyrrolidinyl.
93. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is morpholinyl.
94. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is —N(CH₃)(iPr).
95. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is —N(iPr)₂.
96. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is

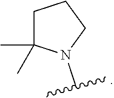 [SMILES: *N1CCCC1(C)C]

97. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is

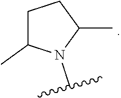

98. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is

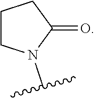 [SMILES: *N1CCCC1=O]

99. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is

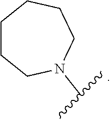 [SMILES: *N1CCCCCC1]

100. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is o

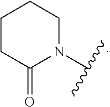 [SMILES: *N1CCCCC1=O]

101. A compound of formula (I) according to numbered embodiment 88, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR8R9) is

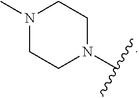 [SMILES: *N1CCN(C)CC1]

102. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—(NR10R7).
103. A compound of formula (I) according to numbered embodiment 102, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein —(NR10R7) is

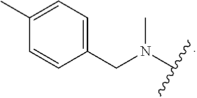 [SMILES: *N(C)Cc1ccc(C)cc1]

104. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—O—(CH₂)ₖ-(aryl).
105. A compound of formula (I) according to numbered embodiment 104, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—O—(CH₂)ₖ-(phenyl).
106. A compound of formula (I) according to numbered embodiment 104, or a tautomer, isomer, stereoisomer (including an enantiomer, a diastereoisomer and a racemic and scalemic mixture thereof), a deuterated isotope, and a pharmaceutically acceptable salt and/or solvate thereof, wherein R4 is

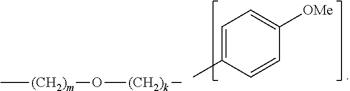

107. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—(SO₂)—(CH₂)ₖ-(aryl).
108. A compound of formula (I) according to numbered embodiment 107, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—(SO₂)—(CH₂)ₖ-(phenyl).
109. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(alkoxy).
110. A compound of formula (I) according to numbered embodiment 109, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(methoxy).
111. A compound of formula (I) according to numbered embodiment 109, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(ethoxy).
112. A compound of formula (I) according to numbered embodiment 109, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(propoxy).
113. A compound of formula (I) according to numbered embodiment 109, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-(butoxy).
114. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—O—(CH₂)ₖ-(heteroaryl).
115. A compound of formula (I) according to numbered embodiment 114, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ—O—(CH₂)ₖ-(pyridyl).
116. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)-[pyridone, which may be optionally substituted by alkyl^{b}, or CF₃].
117. A compound of formula (I) according to numbered embodiment 116, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[pyridonyl].
118. A compound of formula (I) according to numbered embodiment 116, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[pyridone, substituted by —CH₃].
119. A compound of formula (I) according to numbered embodiment 116, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[pyridone, substituted by -(iPr)].
120. A compound of formula (I) according to numbered embodiment 116, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R4 is —(CH₂)ₘ-[pyridone, substituted by —CF₃].
121. A compound of formula (I) according to any one of numbered embodiment 104 to 108, 114 to 115, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein k=0.
122. A compound of formula (I) according to any one of numbered embodiment 104 to 108, 114 to 115, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein k=1.
123. A compound of formula (I) according to any one of numbered embodiment 104 to 108, 114 to 115, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein k=2.
124. A compound of formula (I) according to any one of numbered embodiment 104 to 108, 114 to 115, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein k=3.
125. A compound of formula (I) according to any one of numbered embodiments 60 to 69, 73 to 78, 80, 88 to 124, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein m=0.
126. A compound of formula (I) according to any one of numbered embodiments 60 to 69, 73 to 78, 80, 88 to 124, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein m=1.
127. A compound of formula (I) according to any one of numbered embodiments 60 to 69, 73 to 78, 80, 88 to 124, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein m=2.
128. A compound of formula (I) according to any one of numbered embodiments 60 to 69, 73 to 78, 80, 88 to 124, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein m=3.
129. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein A is —SO₂R6.
130. A compound of formula (I) according to numbered embodiment 129, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is alkyl.
131. A compound of formula (I) according to numbered embodiment 130, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is methyl.
132. A compound of formula (I) according to numbered embodiment 130, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is ethyl.
133. A compound of formula (I) according to numbered embodiment 130, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is propyl.
134. A compound of formula (I) according to numbered embodiment 129, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is (CH₂)₀₋₃-(aryl).
135. A compound of formula (I) according to numbered embodiment 134, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is (CH₂)-(phenyl).
136. A compound of formula (I) according to numbered embodiment 134, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is naphthyl.
137. A compound of formula (I) according to numbered embodiment 134, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is

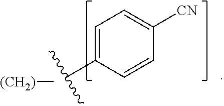

138. A compound of formula (I) according to numbered embodiment 134, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R6 is dimethyl-substituted phenyl.
139. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein A is —(CH₂)—R13.
140. A compound of formula (I) according to numbered embodiment 139, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R13 is heteroaryl.
141. A compound of formula (I) according to numbered embodiment 139, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R13 is cycloalkyl.
142. A compound of formula (I) according to numbered embodiment 139, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R13 is heterocyclyl.
143. A compound of formula (I) according to numbered embodiment 142, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R13 is piperidinyl.
144. A compound of formula (I) according to numbered embodiment 139, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R13 is aryl^{b}.
145. A compound of formula (I) according to numbered embodiment 144, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R13 is

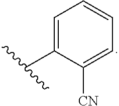 [SMILES: *c1ccccc1C#N]

146. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 is H or alkyl.
147. A compound of formula (I) according numbered embodiment 146, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 is H.
148. A compound of formula (I) according to numbered embodiments 146, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 is alkyl.
149. A compound of formula (I) according to numbered embodiment 148, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 is methyl.
150. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is selected from H, alkyl, —(CH₂)₀₋₃aryl, —(CH₂)O₃heteroaryl, —(CH₂)₀₋₃cycloalkyl, —(CH₂)₀₋₃-[benzothiophene], —(CH₂)₀₋₃-[indole], and

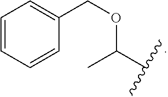 [SMILES: *C(C)OCc1ccccc1]

151. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is H.
152. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is alkyl.
153. A compound of formula (I) according to numbered embodiment 152, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is methyl.
154. A compound of formula (I) according to numbered embodiment 152, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is ethyl.
155. A compound of formula (I) according to numbered embodiment 152, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —CH(OH)CH₃.
156. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is

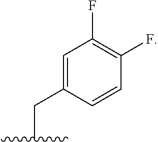 [SMILES: *Cc1ccc(F)c(F)c1]

158. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)-[naphthyl].
159. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)-[mono-, or di-chlorophenyl].
160. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)-[mono-, or di-fluorophenyl].
161. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)-phenyl.
162. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)₂-phenyl.
163. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is

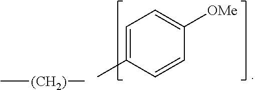

164. A compound of formula (I) according to numbered embodiment 156, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)-biphenyl.
165. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)₀₋₃heteroaryl.
166. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)₀₋₃cycloalkyl.
167. A compound of formula (I) according to numbered embodiment 166, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)-cyclohexyl.
168. A compound of formula (I) according to numbered embodiment 166, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is

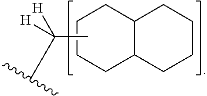

169. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)₀₋₃-[benzothiophene].
170. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is —(CH₂)₀₋₃-[indole].
171. A compound of formula (I) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} is

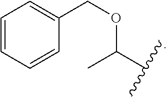 [SMILES: *C(C)OCc1ccccc1]

172. A compound of formula (I) according to any one of numbered embodiments 1 to 145, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 and R2^{A}, together with nitrogen atom to which R1 is attached and the carbon atom to which R2^{A} is attached, are linked by alkylene to form a 4-, 5-, or 6-membered saturated heterocycle.
173. A compound of formula (I) according to numbered embodiment 172, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 and R2^{A}, together with nitrogen atom to which R1 is attached and the carbon atom to which R2^{A} is attached, are linked by alkylene to form a 4-membered saturated heterocycle.
174. A compound of formula (I) according to numbered embodiment 172, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 and R2^{A}, together with nitrogen atom to which R1 is attached and the carbon atom to which R2^{A} is attached, are linked by alkylene to form a 5-membered saturated heterocycle.
175. A compound of formula (I) according to numbered embodiment 172, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R1 and R2^{A}, together with nitrogen atom to which R1 is attached and the carbon atom to which R2^{A} is attached, are linked by alkylene to form a 6-membered saturated heterocycle.
176. A compound of formula (I) according to any one of numbered embodiments 172 to 176, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the saturated heterocycle is substituted with aryl.
177. A compound of formula (I) according to numbered embodiment 176, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the saturated heterocycle is substituted with

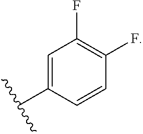 [SMILES: *c1ccc(F)c(F)c1]

178. A compound of formula (I) according to any one of numbered embodiments 172 to 176, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein two adjacent carbon atoms on the saturated heterocycle are linked to form a 6-membered aromatic ring.
179. A compound of formula (I) according to any one of numbered embodiments 172 to 176, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein two adjacent carbon atoms on the saturated heterocycle are linked to form a 3-, 4-, or 5-membered saturated cycloalkyl ring.
180. A compound of formula (I) according to numbered embodiment 179, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein the 3-, 4-, or 5-membered saturated cycloalkyl ring is mono- or di-substituted by alkyl.
181. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein Y is a bond.
182. A compound of formula (I) according to any one of numbered embodiments 1 to 180, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein Y is —[CHR5]-; and R5 is H.
183. A compound of formula (I) according to any one of numbered embodiments 1 to 150, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein Y is —[CHR5]-; and
   together with the carbon atoms to which each of R5 and R2^{A} are attached; R5 and R2^{A} are linked by alkylene to form a 4-, 5-, 6-membered saturated ring.
184. A compound of formula (I) according to any one of numbered embodiments 1 to 146 and 150 to 171, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein Y is —[CHR5]-; and
   together with the nitrogen atom to which R1 is attached, the carbon atom to which R5 is attached, and the carbon atom to which R2^{A} and R2^{B} are both attached, R5 and R1 are linked by alkylene to form a saturated 4-, 5-, or 6-membered heterocycle.
185. A compound of formula (I) according to any one of numbered embodiments 1 to 146, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein Y is —[CHR5]-; and
   together with the nitrogen atom to which R1 is attached, the carbon atom to which R5 is attached, and the carbon atom to which R2^{A} and R2^{B} are both attached, R5 and R1 are linked by alkylene to form a saturated 4-, 5-, or 6-membered heterocycle;
   wherein one atom on the saturated 4-, 5-, or 6-membered heterocycle is linked by alkylene to join with R2^{A}.
186. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{B} is H or alkyl^{b}.
187. A compound of formula (I) according to numbered embodiment 186, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{B} is H.
188. A compound of formula (I) according to numbered embodiment 186, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{B} is alkyl^{b}.
189. A compound of formula (I) according to numbered embodiment 188, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{B} is methyl.
190. A compound of formula (I) according to any one of numbered embodiments 1 to 149, 181 and 182, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} and R2^{B}, together with the carbon to which R2^{A} and R2^{B} are both attached, are linked by alkylene or heteroalkylene to form a 3-, 4-, 5-, or 6-membered saturated ring.
191. A compound of formula (I) according to numbered embodiment 190, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} and R2^{B}, together with the carbon to which R2^{A} and R2^{B} are both attached, are linked by alkylene to form a 3-membered saturated ring.
192. A compound of formula (I) according to numbered embodiment 190, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} and R2^{B}, together with the carbon to which R2^{A} and R2^{B} are both attached, are linked by alkylene to form a 4-membered saturated ring.
193. A compound of formula (I) according to numbered embodiment 190, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} and R2^{B}, together with the carbon to which R2^{A} and R2^{B} are both attached, are linked by heteroalkylene to form a 3-, 4-, 5-, or 6-membered saturated ring containing one or two ring members that are selected from N and O.
194. A compound of formula (I) according to numbered embodiment 190, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R2^{A} and R2^{B}, together with the carbon to which R2^{A} and R2^{B} are both attached, are linked by heteroalkylene to form a 6-membered saturated ring containing O.
195. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing one heteroatom selected from S and N, wherein at least one of the rings is aromatic and, optionally the bicyclic ring contains one additional heteroatom independently selected from N, O and S;
   optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring.
196. A compound of formula (I) according to numbered embodiment 195, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing a N atom and, optionally the bicyclic ring contains one additional heteroatom independently selected from N, and O.
197. A compound of formula (I) according to numbered embodiment 195, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing one S atom, wherein at least one of the rings is aromatic;
   wherein the fused 6,5- or 6,6-bicyclic ring is substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring.
198. A compound of formula (I) according to numbered embodiment 197, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is

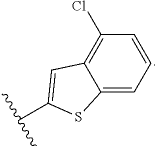 [SMILES: *c1cc2c(Cl)cccc2s1]

199. A compound of formula (I) according to any one of numbered embodiments 195 to 196, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing two N atoms, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring.
200. A compound of formula (I) according to numbered embodiment 199, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

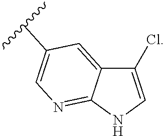 [SMILES: *c1cnc2[nH]cc(Cl)c2c1]

201. A compound of formula (I) according to numbered embodiment 199, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

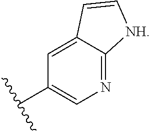 [SMILES: *c1cnc2[nH]ccc2c1]

202. A compound of formula (I) according to numbered embodiment 199, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

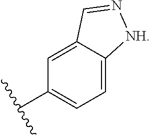 [SMILES: *c1ccc2[nH]ncc2c1]

203. A compound of formula (I) according to numbered embodiment 199, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

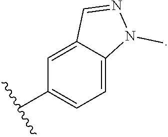 [SMILES: *c1ccc2c(cnn2C)c1]

204. A compound of formula (I) according to numbered embodiment 199, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

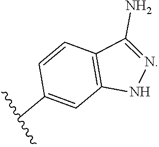 [SMILES: *c1ccc2c(N)n[nH]c2c1]

205. A compound of formula (I) according to numbered embodiment 195 to 196, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing one N atom, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring.
206. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

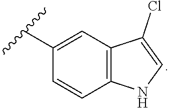 [SMILES: *c1ccc2[nH]cc(Cl)c2c1]

207. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

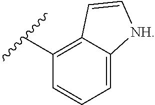 [SMILES: *c1cccc2[nH]ccc12]

208. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

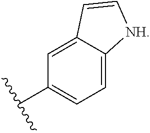 [SMILES: *c1ccc2[nH]ccc2c1]

209. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

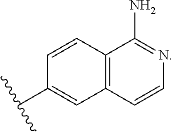 [SMILES: *c1ccc2c(N)nccc2c1]

210. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

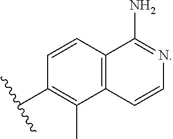 [SMILES: *c1ccc2c(N)nccc2c1C]

211. A compound of formula (I) according to numbered embodiment 205, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

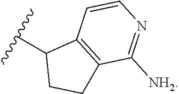 [SMILES: *C1CCc2c1ccnc2N]

212. A compound of formula (I) according to numbered embodiment 195, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing one N atom and one S atom, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring.
213. A compound of formula (I) according to numbered embodiment 212, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

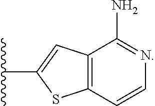 [SMILES: *c1cc2c(N)nccc2s1]

214. A compound of formula (I) according to numbered embodiment 195 to 196, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is a fused 6,5- or 6,6-bicyclic ring, containing one N atom and one O atom, wherein at least one of the rings is aromatic, optionally wherein the fused 6,5- or 6,6-bicyclic ring may be substituted with 1, 2, or 3 substituents selected from alkyl^{b}, alkoxy, OH, NH₂, halo, CN, and CF₃;
   wherein the fused 6,5-bicyclic ring may be attached via the 6- or 5-membered ring.
215. A compound of formula (I) according to numbered embodiment 214, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

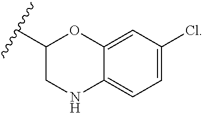 [SMILES: *C1CNc2ccc(Cl)cc2O1]

216. A compound of formula (I) according to numbered embodiment 214, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

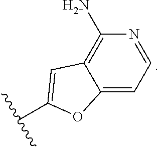 [SMILES: *c1cc2c(N)nccc2o1]

217. A compound of formula (I) according to numbered embodiment 214, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

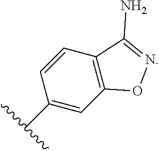 [SMILES: *c1ccc2c(N)noc2c1]

218. A compound of formula (I) according to any one of numbered embodiments 1 to 194, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is phenyl, pyridyl, or thiophenyl, which may be optionally substituted with 1, 2 or 3 substituents independently selected from alkyl^{b}, alkoxy, OH, NH₂ halo, CN, CF₃, —C(═NH)NH₂, and heteroaryl^{b}.
219. A compound of formula (I) according to numbered embodiment 218, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is phenyl which may be optionally substituted with 1, 2 or 3 substituents independently selected from alkyl^{b}, alkoxy, OH, NH₂ halo, CN, CF₃, —C(═NH)NH₂, and heteroaryl^{b}.
220. A compound of formula (I) according to numbered embodiment 219, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is phenyl substituted with alkyl^{b}.
221. A compound of formula (I) according to numbered embodiment 220, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

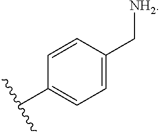 [SMILES: *c1ccc(CN)cc1]

222. A compound of formula (I) according to numbered embodiment 219, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

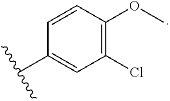 [SMILES: *c1ccc(OC)c(Cl)c1]

223. A compound of formula (I) according to numbered embodiment 219, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

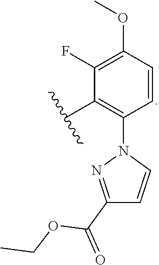 [SMILES: *c1c(-n2ccc(C(=O)OCC)n2)ccc(OC)c1F]

224. A compound of formula (I) according to numbered embodiment 219, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

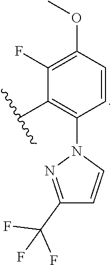 [SMILES: *c1c(-n2ccc(C(F)(F)F)n2)ccc(OC)c1F]

225. A compound of formula (I) according to numbered embodiment 219, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

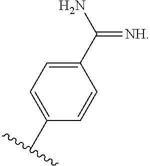 [SMILES: *c1ccc(C(=N)N)cc1]

226. A compound of formula (I) according to numbered embodiment 218, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is pyridyl which may be optionally substituted with 1, 2 or 3 substituents independently selected from alkyl^{b}, alkoxy, OH, NH₂ halo, CN, CF₃, —C(═NH)NH₂, and heteroaryl^{b}.
227. A compound of formula (I) according to numbered embodiment 226, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is pyridyl substituted NH₂.
228. A compound of formula (I) according to numbered embodiment 227, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

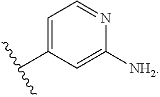 [SMILES: *c1ccnc(N)c1]

229. A compound of formula (I) according to numbered embodiment 218, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

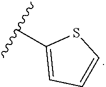 [SMILES: *c1cccs1]

231. A compound of formula (I) according to numbered embodiment 229, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

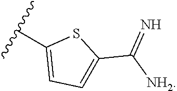 [SMILES: *c1ccc(C(=N)N)s1]

232. A compound of formula (I) according to any one of numbered embodiments 1 to 194, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein R3 is

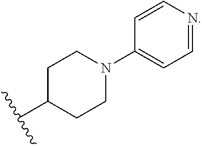 [SMILES: *C1CCN(c2ccncc2)CC1]

233. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein chiral centre *1 is in the (S)-configuration.
234. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
   wherein chiral centre *2 is in the (R)-configuration.
235. A compound selected from any one of Tables 1 to 23, or a pharmaceutically acceptable salt, solvate or solvate or a salt thereof.
236. A compound according to any preceding numbered embodiment.
237. A pharmaceutically acceptable salt according to any of numbered embodiments 1 to 235.
238. A pharmaceutically acceptable solvate according to any of numbered embodiments 1 to 235.
239. A pharmaceutically acceptable solvate of a salt according to any of numbered embodiments 1 to 235.
240. A pharmaceutical composition comprising:
   (i) a compound according to numbered embodiment 236, the pharmaceutically acceptable salt according to numbered embodiment 237, the pharmaceutically acceptable solvate according to numbered embodiment 238, or the pharmaceutically acceptable solvate of a salt according to numbered embodiment 239; and
   (ii) at least one pharmaceutically acceptable excipient.
241. A compound as defined in numbered embodiment 236, a pharmaceutically acceptable salt according to numbered embodiment 237, a pharmaceutically acceptable solvate according to numbered embodiment 238, a pharmaceutically acceptable solvate of a salt according to numbered embodiment 239, or the pharmaceutical composition as defined in numbered embodiment 240, for use in medicine.
242. The use of a compound as defined in numbered embodiment 236, a pharmaceutically acceptable salt according to numbered embodiment 237, a pharmaceutically acceptable solvate according to numbered embodiment 238, a pharmaceutically acceptable solvate of a salt according to numbered embodiment 239, or the pharmaceutical composition as defined in numbered embodiment 240, in the manufacture of a medicament for the treatment or prevention of a disease or condition in which Factor XIIa activity is implicated.
243. A method of treatment of a disease or condition in which Factor XIIa activity is implicated comprising administration to a subject in need thereof a therapeutically effective amount of a compound as defined in numbered embodiment 236, a pharmaceutically acceptable salt according to numbered embodiment 237, a pharmaceutically acceptable solvate according to numbered embodiment 238, a pharmaceutically acceptable solvate of a salt according to numbered embodiment 239, or the pharmaceutical composition as defined in numbered embodiment 240.
244. A compound as defined in numbered embodiment 236, a pharmaceutically acceptable salt according to numbered embodiment 237, a pharmaceutically acceptable solvate according to numbered embodiment 238, a pharmaceutically acceptable solvate of a salt according to numbered embodiment 239, or a pharmaceutical composition as defined in numbered embodiment 240, for use in a method of treatment of a disease or condition in which Factor XIIa activity is implicated.
245. The use of numbered embodiment 242, the method of numbered embodiment 243, or a compound, a pharmaceutically acceptable salt, a pharmaceutically acceptable solvate, a pharmaceutically acceptable solvate of a salt, or a pharmaceutical composition for use as defined in numbered embodiment 244, wherein, the disease or condition in which Factor XIIa activity is implicated is a bradykinin-mediated angioedema.
246. The use of numbered embodiment 245, the method of numbered embodiment 245 or a compound, a pharmaceutically acceptable salt, a pharmaceutically acceptable solvate, a pharmaceutically acceptable solvate of a salt, or a pharmaceutical composition for use as defined in numbered embodiment 245, wherein the bradykinin-mediated angioedema is hereditary angioedema.
247. The use of numbered embodiment 245, the method of numbered embodiment 245 or a compound, a pharmaceutically acceptable salt, a pharmaceutically acceptable solvate, a pharmaceutically acceptable solvate of a salt, or a pharmaceutical composition for use as defined in numbered embodiment 245, wherein the bradykinin-mediated angioedema is non hereditary.
248. The use of numbered embodiment 242, the method of numbered embodiment 243, or a compound, a pharmaceutically acceptable salt, a pharmaceutically acceptable solvate, a pharmaceutically acceptable solvate of a salt, or a pharmaceutical composition for use as defined in numbered embodiment 244, wherein the disease or condition in which Factor XIIa activity is implicated is selected from vascular hyperpermeability, stroke including ischemic stroke and haemorrhagic accidents; retinal edema; diabetic retinopathy; DME; retinal vein occlusion; and AMD.
249. The use of numbered embodiment 242, the method of numbered embodiment 243, or a compound, a pharmaceutically acceptable salt, a pharmaceutically acceptable solvate, a pharmaceutically acceptable solvate of a salt, or a pharmaceutical composition for use as defined in numbered embodiment 244, wherein the disease or condition in which Factor XIIa activity is implicated is a thrombotic disorder.
250. The use of numbered embodiment 249, the method of numbered embodiment 249, or a compound, a pharmaceutically acceptable salt, a pharmaceutically acceptable solvate, a pharmaceutically acceptable solvate of a salt, or a pharmaceutical composition for use as defined in numbered embodiment 249, wherein the thrombotic disorder is thrombosis; thromboembolism caused by increased propensity of medical devices that come into contact with blood to clot blood; prothrombotic conditions such as disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.
251. The use of numbered embodiment 242, the method of numbered embodiment 243, or a compound, a pharmaceutically acceptable salt, a pharmaceutically acceptable solvate, a pharmaceutically acceptable solvate of a salt, or a pharmaceutical composition for use as defined in numbered embodiment 244, wherein, the disease or condition in which Factor XIIa activity is implicated is selected from neuroinflammation;
   neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.
252. The use of numbered embodiments 242 and 245 to 251, the method of numbered embodiment 243 and 245 to 251, or a compound, a pharmaceutically acceptable salt, a pharmaceutically acceptable solvate, a pharmaceutically acceptable solvate of a salt, or a pharmaceutical composition for use as defined in numbered embodiment 244 and 245 to 251, wherein the compound targets FXIIa.
253. The use of numbered embodiments 242 and 245 to 252, the method of numbered embodiment 243 and 245 to 252, or a compound, a pharmaceutically acceptable salt, a pharmaceutically acceptable solvate, a pharmaceutically acceptable solvate of a salt, or a pharmaceutical composition for use as defined in numbered embodiment 244 and 245 to 252, wherein the compound or pharmaceutical compound is administered parenterally.
254. The use of numbered embodiments 242 and 245 to 253, the method of numbered embodiment 243 and 245 to 253, or a compound, a pharmaceutically acceptable salt, a pharmaceutically acceptable solvate, a pharmaceutically acceptable solvate of a salt, or a pharmaceutical composition for use as defined in numbered embodiment 244 and 245 to 253, wherein the compound of pharmaceutical composition is administered in a form suitable for injection.
255. The use of numbered embodiments 242 and 245 to 254, the method of numbered embodiment 243 and 245 to 254, or a compound, a pharmaceutically acceptable salt, a pharmaceutically acceptable solvate, a pharmaceutically acceptable solvate of a salt, or a pharmaceutical composition for use as defined in numbered embodiment 244 and 245 to 254, wherein the compound of pharmaceutical composition is administered in a form suitable for intra-vitreal injection.

1. A compound of formula (I): 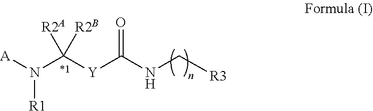
wherein
*1 denotes a chiral centre
n is 0, 1 or 2;
A is selected from H, —(C═O)R4, —SO₂R6, or —(CH₂)—R13;
Y is either a bond, or —[CHR5]-;
R1 is H or alkyl^{b};
R2^{A} is selected from H, alkyl, —(CH₂)₀₋₃aryl, —(CH₂)₀₋₃heteroaryl, —(CH₂)₀₋₃cycloalkyl, —(CH₂)₀₋₃-[benzothiophene], —(CH₂)₀₋₃-[indole], or 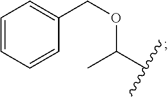 [SMILES: *C(C)OCc1ccccc1]
or,
when Y is a bond, R1 and R2^{A}, together with nitrogen atom to which R1 is attached and the carbon atom to which R2^{A} is attached, is optionally linked by alkylene to form a 4-, 5-, or 6-membered saturated heterocycle, optionally wherein the 4-, 5-, or 6-membered saturated heterocycle is optionally substituted with aryl, or wherein two adjacent carbon atoms on the 4-, 5-, or 6-membered saturated heterocycle are optionally linked to form a 6-membered aromatic ring, or wherein two adjacent carbon atoms on the 4-, 5-, or 6-membered saturated heterocycle are optionally linked to form a 3-, 4-, or 5-membered saturated hydrocarbon ring which is optionally mono- or di-substituted by alkyl^{b};
when Y is —[CHR5]-, R5 is H; or,
when Y is —[CHR5]-, together with the carbon atoms to which each of R5 and R2^{A} are attached, R5 and R2^{A} is optionally linked by alkylene to form a 4-, 5-, 6-membered saturated ring; or,
when Y is —[CHR5]-, together with the nitrogen atom to which R1 is attached, the carbon atom to which R5 is attached, and the carbon atom to which R2^{A} and R2^{B} are both attached, R5 and R1 is optionally linked by alkylene to form a saturated 4-, 5-, or 6-membered heterocycle, optionally wherein one atom on the saturated 4-, 5-, or 6-membered heterocycle is optionally linked by alkylene to join with R2^{A};
R2^{B} is H or alkyl^{b}; or,
R2^{A} and R2^{B}, together with the carbon to which R2^{A} and R2^{B} are both attached, is linked by alkylene or heteroalkylene to form a 3-, 4-, 5-, or 6-membered saturated ring, optionally wherein the 3-, 4-, 5-, or 6-membered saturated ring contains one or two ring members that are selected from N or O;
R3 is:
(i) a fused 6,5- or 6,6-bicyclic ring, containing one heteroatom selected from S or N, wherein at least one of the rings is aromatic and, optionally the bicyclic ring contains one additional heteroatom independently selected from N, O or S;
optionally wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring is optionally attached via the 6- or 5-membered ring; or
(ii) phenyl, pyridyl, or thiophenyl, which is optionally substituted with 1, 2 or 3 substituents independently selected from alkyl^{b}, alkoxy, OH, NH₂ halo, CN, CF₃, —C(═NH)NH₂, or heteroaryl^{b};
wherein when n=1, and R3 is phenyl substituted with at least one —(CH₂NH₂), R2^{A} is alkyl and R2^{B} is H; or
(iii) 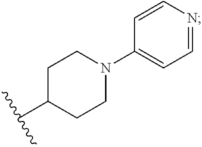 [SMILES: *C1CCN(c2ccncc2)CC1]
R4 is one of:
(i) a group of formula (II), 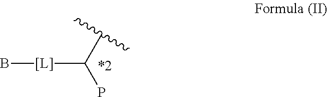
wherein -[L]- is a bond, —[(CH₂)₁₋₄]—, —[(CH₂)—O—(CH₂)]—, or —[O—(CH₂)]—; and P is alkoxy, OH or NR11R12;
wherein *2 denotes a chiral centre, and
wherein when -[L]- is a bond, B is a C₁₋₄ linear or branched chain hydrocarbon, and
wherein when -[L]- is —[(CH₂)₁₋₄]—, —[(CH₂)—O—(CH₂)]—, or —[O—(CH₂)]—, B is OH, aryl, heteroaryl, heterocyclyl, cycloalkyl or 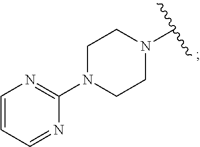 [SMILES: *N1CCN(c2ncccn2)CC1]
or,
(ii) —(CH₂)ₘ-[fused 6,5- or 6,6-heteroaromatic bicyclic ring], wherein at least one ring atom is a heteroatom selected from O, N or S, and optionally, 1, 2 or 3 additional ring atoms are selected from N or NH; wherein the fused 6,5- or 6,6-heteroaromatic bicyclic ring is optionally substituted with 1, 2 or 3 alkyl^{b}; wherein the 6,5-heteroaromatic bicyclic ring is optionally attached to —(CH₂)ₘ— via the 6- or 5-membered ring; or,
(iii) methyl, —C(CH₃)₂(OH), —C(CH₃)₂(NHMe), —(CH₂)ₘ-(aryl), —(CH₂)ₘ-(cycloalkyl), —(CH₂)ₘ-(heteroaryl), —(CH₂)ₘ-(heterocyclyl), —(CH₂)-(alkyl), —(CH(halo)₂), —(CH₂)ₘ—(NR8R9), —(CH₂)ₘ—(NR10R7), —(CH₂)ₘ—O—(CH₂)ₖ-(aryl), —(CH₂)ₘ—(SO₂)—(CH₂)ₖ-(aryl), —(CH₂)ₘ-(alkoxy), —(CH₂)ₘ—O—(CH₂)ₖ-(heteroaryl), or —(CH₂)ₘ-[pyridone, which is optionally substituted by alkyl^{b}, or CF₃];
wherein k=0, 1, 2, or 3;
wherein m=0, 1, 2 or 3;
wherein:
when Y is —[CHR5]- and R5 is H, R2^{A} is CH₂-aryl or H; and
when Y is —[CHR5]-, R3 is 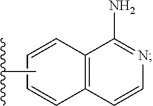
when A is H, R3 is 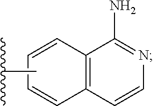
and
when R3 is 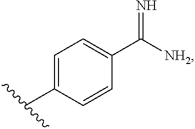 [SMILES: *c1ccc(C(=N)N)cc1]
R2^{A} is not H;
wherein:
R6 is alkyl or —(CH₂)₀₋₃-(aryl);
R7 is independently selected from H, —SO₂CH₃, methyl, ethyl, propyl, isopropyl, or cycloalkyl;
R8 and R9 are independently selected from H, —SO₂CH₃, alkyl^{b}, heteroaryl^{b}, or cycloalkyl; or R8 and R9 together with the nitrogen atom to which they are attached form a carbon-containing 4-, 5-, 6- or 7-membered heterocyclic ring, optionally containing an additional heteroatom selected from N, NR10, S, or O, which is saturated or unsaturated with 1 or 2 double bonds and which are optionally mono- or di-substituted with substituents independently selected from oxo, alkyl^{b}, alkoxy, OH, halo, —SO₂CH₃, or CF₃; or R8 and R9 together with the nitrogen atom to which they are attached form a carbon-containing 5- or 6-membered heterocyclic ring, which is fused to an aryl^{b} or a heteroaryl^{b};
R10 is independently selected from H, —SO₂R6, alkyl^{b}, —(CH₂)₀₋₃aryl^{b}, —(CH₂)₀₋₃heteroaryl^{b}, cycloalkyl, —(C═O)-(aryl), or —(CH₂)₀₋₃heterocyclyl^{b}; or R10 is a carbon-containing 4-, 5-, 6- or 7-membered heterocyclic ring, optionally containing an additional heteroatom selected from N, NR7, S, SO, SO₂, or O, which is saturated or unsaturated with 1 or 2 double bonds and which is optionally mono- or di-substituted with substituents independently selected from oxo, alkyl^{b}, alkoxy, OH, halo, —SO₂CH₃, or CF₃;
R11 and R12 are independently selected from H, alkyl^{b}, —SO₂R6, cycloalkyl, —(C═O)O-(alkyl^{b}), —(C═O)-phenyl, —CH₂-phenyl, or CH₂—COOH; or R11 and R12 together with the nitrogen atom to which they are attached form a carbon-containing 4-, 5-, 6- or 7-membered heterocyclic ring optionally containing an additional heteroatom selected from N, O, or NR10, wherein the heterocyclic ring is optionally mono- or di-substituted with substituents independently selected from alkyl^{b}, OH, halo or CF₃;
R13 is selected from heteroaryl, cycloalkyl, heterocyclyl or aryl^{b};
wherein:
alkoxy is a linear O-linked hydrocarbon of between 1 and 6 carbon atoms (C₁-C₆) or a branched O-linked hydrocarbon of between 3 and 6 carbon atoms (C₃-C₆); alkoxy is optionally substituted with 1 or 2 substituents independently selected from OH, CN, CF₃, —N(R7)₂ or fluoro;
alkyl is a linear saturated hydrocarbon having up to 6 carbon atoms (C₁-C₆) or a branched saturated hydrocarbon of between 3 and 6 carbon atoms (C₃-C₆); alkyl is optionally substituted with 1 or 2 substituents independently selected from (C₁-C₆)alkoxy, OH, —NR8R9, —NHCOCH₃, —CO(heterocyclyl^{b}), —COOR8, —CONR8R9, CN, CF₃, halo, oxo or heterocyclyl^{b};
alkyl^{b} is a linear saturated hydrocarbon having up to 6 carbon atoms (C₁-C₆) or a branched saturated hydrocarbon of between 3 and 6 carbon atoms (C₃-C₆); alkyl is optionally substituted with 1 or 2 substituents independently selected from (C₁-C₆)alkoxy, OH, —N(R7)₂, —NHCOCH₃, CF₃, halo, oxo o cyclopropane;
alkylene is a bivalent linear saturated hydrocarbon having 1 to 5 carbon atoms (C₁-C₆); alkylene is optionally substituted with 1 or 2 substituents independently selected from alkyl, (C₁-C₆)alkoxy, OH, CN, CF₃ r halo;
aryl is phenyl, biphenyl or naphthyl; aryl is optionally substituted with 1, 2 or 3 substituents independently selected from alkyl, alkoxy, OH, —SO₂CH₃, halo, —SO₂NR8R9, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR8, —CONR8R9, —(CH₂)₀₋₃—NR8R9, OCF₃ or CF₃; or two adjacent carbon ring atoms on the aryl are optionally linked by a heteroalkylene to form a non-aromatic ring containing 5, 6, or 7 ring members which is optionally substituted with OH; or optionally wherein two adjacent ring atoms on aryl are linked to form a 5- or 6-membered aromatic ring containing 1 or 2 heteroatoms that are selected from N, NR10, S, or O;
aryl^{b} is phenyl, biphenyl or naphthyl, which is optionally substituted with 1, 2 or 3 substituents independently selected from methyl, ethyl, propyl, isopropyl, alkoxy, OH, —SO₂CH₃, N(R7)₂, halo, CN, or CF₃; or two adjacent carbon ring atoms on the aryl is optionally linked by a heteroalkylene to form a non-aromatic ring containing 5, 6, or 7 ring members;
cycloalkyl is monocyclic saturated hydrocarbon ring of between 3 and 6 carbon atoms (C₃-C₆); cycloalkyl is optionally substituted with 1 or 2 substituents independently selected from methyl, ethyl, propyl, isopropyl, methoxy, ethoxy, propoxy, isopropoxy, OH, CN, CF₃ or halo; optionally wherein two adjacent ring atoms on cycloalkyl are linked to form a 5- or 6-membered saturated hydrocarbon ring;
halo is F, Cl, Br, or I;
heteroalkylene is a bivalent linear saturated hydrocarbon having 2 to 5 carbon atoms (C₂-C₅), wherein 1 or 2 of the 2 to 5 carbon atoms are replaced with NR10, S, or O; heteroalkylene is optionally substituted with 1 or 2 substituents independently selected from alkyl, (C₁-C₆)alkoxy, OH, CN, CF₃ or halo;
heteroaryl is a 5- or 6-membered carbon-containing aromatic ring containing one, two or three ring members that are selected from N, NR10, S, or O; heteroaryl is optionally substituted with 1, 2 or 3 substituents independently selected from alkyl, alkoxy, heteroaryl^{b}, phenyl, cycloalkyl, OH, OCF₃, halo, heterocyclyl^{b}, CN, or CF₃;
heteroaryl^{b} is a 5- or 6-membered carbon-containing aromatic ring containing one, two or three ring members that are selected from N, NR10, S, or O; heteroaryl^{b} is optionally substituted with 1, 2 or 3 substituents independently selected from methyl, ethyl, propyl, isopropyl, alkoxy, OH, OCF₃, COOCH₃, COOCH₂CH₃, COO—(CH₂)₂—CH₃, COO-(iPr), halo, CN, or CF₃;
heterocyclyl is a 4-, 5-, 6-, or 7-membered carbon-containing non-aromatic ring containing one, two, three, or four ring members that are selected from N, NR10, S, SO, SO₂ or O; heterocyclyl is optionally substituted with 1, 2, 3, or 4 substituents independently selected from alkyl, alkoxy, aryl^{b}, OH, OCF₃, halo, oxo, CN, NR8R9, —O(aryl^{b}), —O(heteroaryl^{b}) or CF₃; or optionally wherein two ring atoms on heterocyclyl are linked with an alkylene to form a non-aromatic ring containing 5, 6, or 7 ring members; or optionally wherein two ring atoms on heterocyclyl are linked with an heteroalkylene to form a non-aromatic ring containing 5, 6, or 7 ring members; or optionally wherein two adjacent ring atoms on heterocyclyl are linked to form a 5- or 6-membered aromatic ring which optionally contains 1 or 2 heteroatoms that are selected from N, NR10, S, or O;
heterocyclyl^{b} is a 4-, 5-, 6-, or 7-membered carbon-containing non-aromatic ring containing one, two or three ring members that are selected from N, NR7, S, SO, S02 or O; heterocyclyl^{b} is optionally substituted with 1, 2, 3, or 4 substituents independently selected from methyl, ethyl, propyl, isopropyl, alkoxy, OH, OCF₃, halo, oxo, CN, or CF₃;
or a tautomer, isomer, stereoisomer, deuterated isotope, pharmaceutically acceptable salt and/or solvate thereof.

2. The compound of formula (I) according to claim 1, or a tautomer, isomer, stereoisomer a deuterated isotope, a pharmaceutically acceptable salt and/or solvate thereof, wherein n is 1.

3. The compound of formula (I) according to claim 1, or a tautomer, isomer, stereoisomer a deuterated isotope, a pharmaceutically acceptable salt and/or solvate thereof, wherein A is —(C═O)R4.

4. The compound of formula (I) according to claim 1, or a tautomer, isomer, stereoisomer a deuterated isotope, a pharmaceutically acceptable salt and/or solvate thereof, wherein R4 is a group of formula (II), 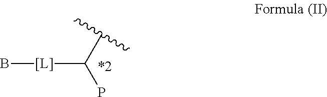
wherein -[L]- is —[(CH₂)₁₋₄]—, —[(CH₂)—O—(CH₂)]—, or —[O—(CH₂)]—;
P is alkoxy, OH or NR11R12;
and B is OH, aryl, heteroaryl, heterocyclyl, cycloalkyl or

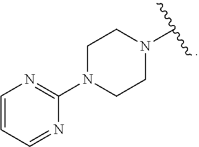 [SMILES: *N1CCN(c2ncccn2)CC1]

5. The compound of formula (I) according to claim 1, or a tautomer, isomer, stereoisomer a deuterated isotope, a pharmaceutically acceptable salt and/or solvate thereof, wherein P is NR11R12.

6. The compound of formula (I) according to claim 1, or a tautomer, isomer, stereoisomer a deuterated isotope, a pharmaceutically acceptable salt and/or solvate thereof, wherein P is NHR12.

7. The compound of formula (I) according to claim 1, or a tautomer, isomer, stereoisomer a deuterated isotope, a pharmaceutically acceptable salt and/or solvate thereof, wherein P is selected from NH₂, NH(alkyl) or NH(cyclohexyl).

8. The compound of formula (I) according to claim 1, or a tautomer, isomer, stereoisomer a deuterated isotope, a pharmaceutically acceptable salt and/or solvate thereof, wherein P is NH₂.

9. The compound of formula (I) according to claim 1, or a tautomer, isomer, stereoisomer a deuterated isotope, a pharmaceutically acceptable salt and/or solvate thereof, wherein P is NH(iPr).

10. The compound of formula (I) according to claim 1, or a tautomer, isomer, stereoisomer a deuterated isotope, a pharmaceutically acceptable salt and/or solvate thereof, wherein P is NH(cyclohexyl).

11. The compound of formula (I) according to claim 1, or a tautomer, isomer, stereoisomer a deuterated isotope, a pharmaceutically acceptable salt and/or solvate thereof, wherein P is N-linked pyrrolidinyl.

12. The compound of formula (I) according to claim 1, or a tautomer, isomer, stereoisomer a deuterated isotope, a pharmaceutically acceptable salt and/or solvate thereof, wherein -[L]- is —[(CH₂)₁₋₄]—.

13. The compound of formula (I) according to claim 1, or a tautomer, isomer, stereoisomer a deuterated isotope, a pharmaceutically acceptable salt and/or solvate thereof, wherein -[L]- is —[(CH₂)₂]—.

14. The compound of formula (I) according to claim 1, or a tautomer, isomer, stereoisomer a deuterated isotope, a pharmaceutically acceptable salt and/or solvate thereof, wherein -[L]- is —[(CH₂)₄]—.

15. The compound of formula (I) according to claim 1, or a tautomer, isomer, stereoisomer a deuterated isotope, a pharmaceutically acceptable salt and/or solvate thereof, wherein B is aryl.

16. The compound of formula (I) according to claim 1, or a tautomer, isomer, stereoisomer a deuterated isotope, a pharmaceutically acceptable salt and/or solvate thereof, wherein B is phenyl.

17. The compound of formula (I) according to claim 1, or a tautomer, isomer, stereoisomer a deuterated isotope, a pharmaceutically acceptable salt and/or solvate thereof, wherein B is heterocyclyl.

18. The compound of formula (I) according to claim 1, or a tautomer, isomer, stereoisomer a deuterated isotope, a pharmaceutically acceptable salt and/or solvate thereof, wherein B is piperidinyl.

19. The compound of formula (I) according to claim 1, or a tautomer, isomer, stereoisomer a deuterated isotope, a pharmaceutically acceptable salt and/or solvate thereof, wherein R4 is —(CH₂)ₘ-(heterocyclyl).

20. The compound of formula (I) according to claim 1, or a tautomer, isomer, stereoisomer a deuterated isotope, a pharmaceutically acceptable salt and/or solvate thereof,
wherein R4 is

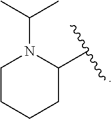 [SMILES: *C1CCCCN1C(C)C]

21. The compound of formula (I) according to claim 1, or a tautomer, isomer, stereoisomer a deuterated isotope, a pharmaceutically acceptable salt and/or solvate thereof, wherein R4 is —(CH₂)ₘ—(NR8R9).

22. The compound of formula (I) according to claim 21, or a tautomer, isomer, stereoisomer a deuterated isotope, a pharmaceutically acceptable salt and/or solvate thereof,
wherein —(NR8R9) is

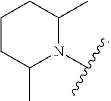 [SMILES: *N1C(C)CCCC1C]

23. The compound of formula (I) according to claim 1, or a tautomer, isomer, stereoisomer a deuterated isotope, a pharmaceutically acceptable salt and/or solvate thereof, wherein A is —SO₂R6.

24. The compound of formula (I) according to claim 1, or a tautomer, isomer, stereoisomer a deuterated isotope, a pharmaceutically acceptable salt and/or solvate thereof, wherein R1 is H.

25. The compound of formula (I) according to claim 1, or a tautomer, isomer, stereoisomer a deuterated isotope, a pharmaceutically acceptable salt and/or solvate thereof,
wherein R2^{A} is selected from H, alkyl, —(CH₂)₀₋₃aryl, —(CH₂)₀₋₃heteroaryl, —(CH₂)₀₋₃cycloalkyl, —(CH₂)₀₋₃-[benzothiophene], —(CH₂)₀₋₃-[indole], or

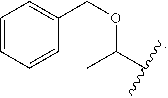 [SMILES: *C(C)OCc1ccccc1]

26. The compound of formula (I) according to claim 1, or a tautomer, isomer, stereoisomer a deuterated isotope, a pharmaceutically acceptable salt and/or solvate thereof, wherein R2^{A} is alkyl.

27. The compound of formula (I) according to claim 1, or a tautomer, isomer, stereoisomer a deuterated isotope, a pharmaceutically acceptable salt and/or solvate thereof, wherein R2^{A} is methyl.

28. The compound of formula (I) according to claim 1, or a tautomer, isomer, stereoisomer a deuterated isotope, a pharmaceutically acceptable salt and/or solvate thereof, wherein R2^{A} is —(CH₂)₀₋₃aryl.

29. The compound of formula (I) according to claim 1, or a tautomer, isomer, stereoisomer a deuterated isotope, a pharmaceutically acceptable salt and/or solvate thereof,
wherein R2^{A} is selected from 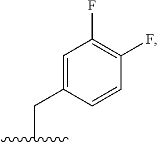 [SMILES: *Cc1ccc(F)c(F)c1]
—(CH₂)-[naphthyl], —(CH₂)-[mono-, or di-chlorophenyl], —(CH₂)-[mono-, or di-fluorophenyl], —(CH₂)-phenyl, —(CH₂)₂-phenyl, 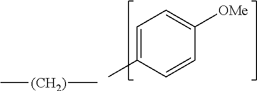
or —(CH₂)-biphenyl.

30. The compound of formula (I) according to claim 1, or a tautomer, isomer, stereoisomer a deuterated isotope, a pharmaceutically acceptable salt and/or solvate thereof,
wherein R2^{A} is

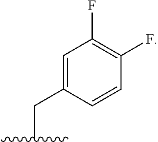 [SMILES: *Cc1ccc(F)c(F)c1]

31. The compound of formula (I) according to claim 1, or a tautomer, isomer, stereoisomer a deuterated isotope, a pharmaceutically acceptable salt and/or solvate thereof,
wherein R1 and R2^{A}, together with nitrogen atom to which R1 is attached and the carbon atom to which R2^{A} is attached, are linked by alkylene to form a 4-, 5-, or 6-membered saturated heterocycle.

32. The compound of formula (I) according to claim 1, or a tautomer, isomer, stereoisomer a deuterated isotope, a pharmaceutically acceptable salt and/or solvate thereof,
wherein R1 and R2^{A}, together with nitrogen atom to which R1 is attached and the carbon atom to which R2^{A} is attached, are linked by alkylene to form a 4-membered saturated heterocycle.

33. The compound of formula (I) according to claim 1, or a tautomer, isomer, stereoisomer a deuterated isotope, a pharmaceutically acceptable salt and/or solvate thereof, wherein Y is a bond.

34. The compound of formula (I) according to claim 1, or a tautomer, isomer, stereoisomer a deuterated isotope, a pharmaceutically acceptable salt and/or solvate thereof,
wherein R2^{A} and R2^{B}, together with the carbon to which R2^{A} and R2^{B} are both attached, are linked by alkylene or heteroalkylene to form a 3-, 4-, 5-, or 6-membered saturated ring.

35. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing one heteroatom selected from S or N, wherein at least one of the rings is aromatic and, optionally the bicyclic ring contains one additional heteroatom independently selected from N, O or S;
optionally wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring is optionally attached via the 6- or 5-membered ring.

36. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing a N atom and, optionally the bicyclic ring contains one additional heteroatom independently selected from N or O.

37. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing one S atom, wherein at least one of the rings is aromatic;
wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring optionally is attached via the 6- or 5-membered ring.

38. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing two N atoms, wherein at least one of the rings is aromatic, optionally wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring is optionally attached via the 6- or 5-membered ring.

39. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing one N atom, wherein at least one of the rings is aromatic, optionally wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring optionally is attached via the 6- or 5-membered ring.

40. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing one N atom and one O atom, wherein at least one of the rings is aromatic, optionally wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring optionally is attached via the 6- or 5-membered ring.

41. The compound of formula (I) according to claim 1, or a tautomer, isomer, stereoisomer a deuterated isotope, a pharmaceutically acceptable salt and/or solvate thereof,
wherein R3 is a fused 6,5- or 6,6-bicyclic ring, containing one N atom and one S atom, wherein at least one of the rings is aromatic, optionally wherein the fused 6,5- or 6,6-bicyclic ring is substituted with 1, 2, or 3 substituents selected from alkyl^{b}, alkoxy, OH, NH₂, halo, CN, or CF₃;
wherein the fused 6,5-bicyclic ring optionally is attached via the 6- or 5-membered ring.

42. The compound of formula (I) according to claim 1, or a tautomer, isomer, stereoisomer a deuterated isotope, a pharmaceutically acceptable salt and/or solvate thereof,
wherein R3 is phenyl, pyridyl, or thiophenyl, which is optionally substituted with 1, 2 or 3 substituents independently selected from alkyl^{b}, alkoxy, OH, NH₂ halo, CN, CF₃, —C(═NH)NH₂, or heteroaryl^{b}.

43. The compound of formula (I) according to claim 1, or a tautomer, isomer, stereoisomer a deuterated isotope, a pharmaceutically acceptable salt and/or solvate thereof,
wherein R3 is

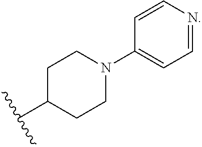 [SMILES: *C1CCN(c2ccncc2)CC1]

44. The compound of formula (I) according to claim 1, or a tautomer, isomer, stereoisomer a deuterated isotope, a pharmaceutically acceptable salt and/or solvate thereof,
wherein R3 is selected from:

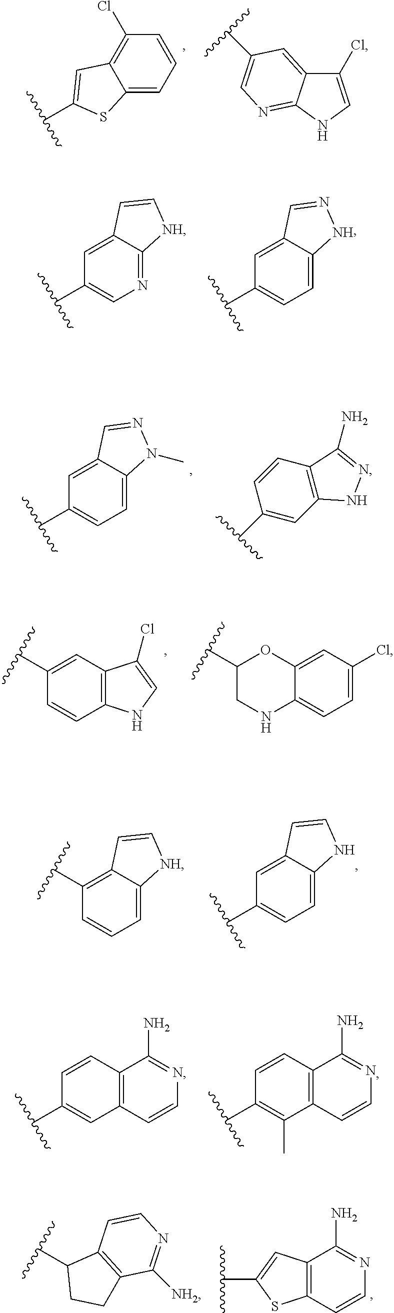

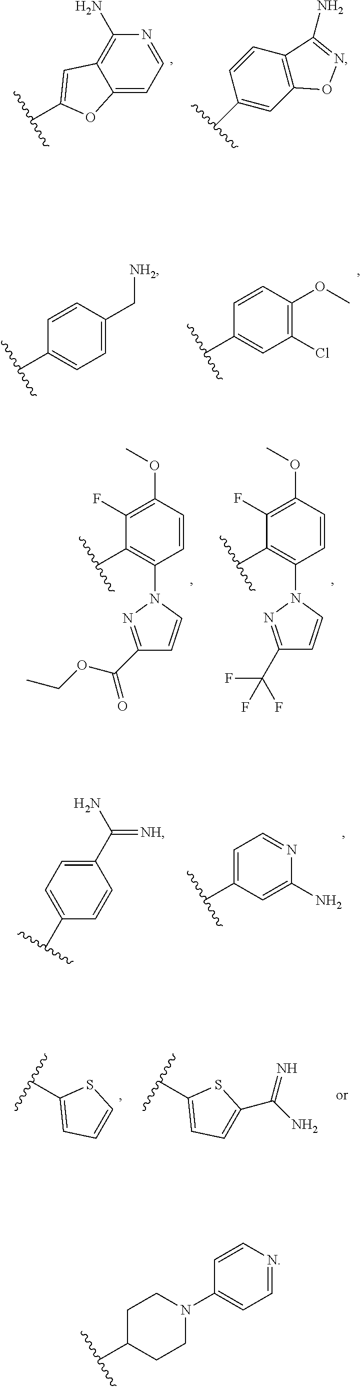

45. The compound of formula (I) according to claim 44, or a tautomer, isomer, stereoisomer a deuterated isotope, a pharmaceutically acceptable salt and/or solvate thereof,
wherein R3 is selected from

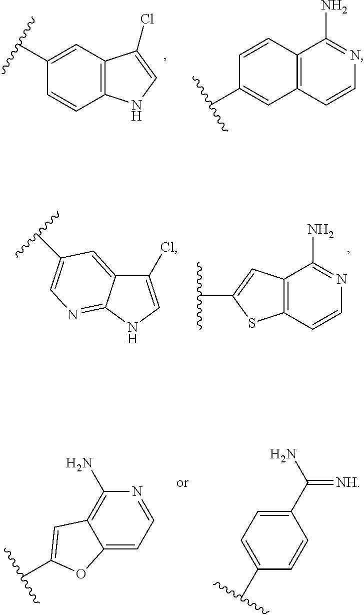

46. The compound of formula (I) according to claim 1, or a tautomer, isomer, stereoisomer a deuterated isotope, a pharmaceutically acceptable salt and/or solvate thereof, wherein chiral centre *1 is in the (S)-configuration.

47. The compound of formula (I) according to claim 1, or a tautomer, isomer, stereoisomer a deuterated isotope, a pharmaceutically acceptable salt and/or solvate thereof, wherein chiral centre *2 is in the (R)-configuration.

48. A compound that is: 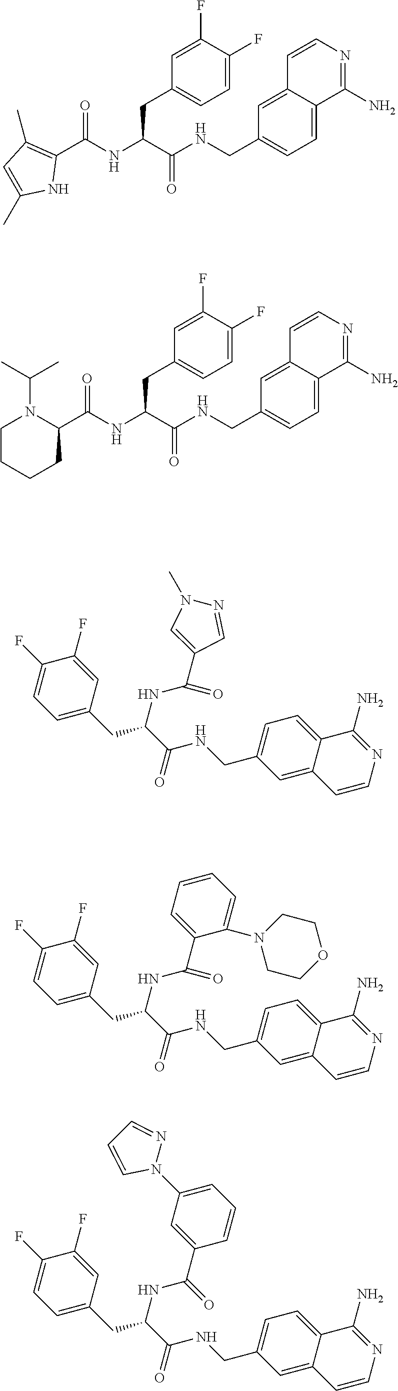 [SMILES: Cc1cc(C)c(C(=O)N[C@@H](Cc2ccc(F)c(F)c2)C(=O)NCc2ccc3c(N)nccc3c2)[nH]1 ; CC(C)N1CCCC[C@@H]1C(=O)N[C@@H](Cc1ccc(F)c(F)c1)C(=O)NCc1ccc2c(N)nccc2c1 ; Cn1cc(C(=O)N[C@@H](Cc2ccc(F)c(F)c2)C(=O)NCc2ccc3c(N)nccc3c2)cn1 ; Nc1nccc2cc(CNC(=O)[C@H](Cc3ccc(F)c(F)c3)NC(=O)c3ccccc3N3CCOCC3)ccc12 ; Nc1nccc2cc(CNC(=O)[C@H](Cc3ccc(F)c(F)c3)NC(=O)c3cccc(-n4cccn4)c3)ccc12] 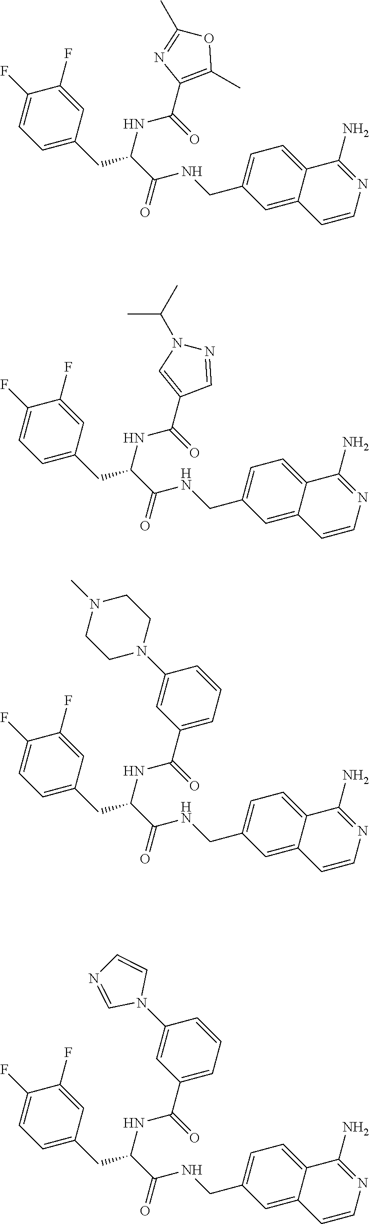 [SMILES: Cc1nc(C(=O)N[C@@H](Cc2ccc(F)c(F)c2)C(=O)NCc2ccc3c(N)nccc3c2)c(C)o1 ; CC(C)n1cc(C(=O)N[C@@H](Cc2ccc(F)c(F)c2)C(=O)NCc2ccc3c(N)nccc3c2)cn1 ; CN1CCN(c2cccc(C(=O)N[C@@H](Cc3ccc(F)c(F)c3)C(=O)NCc3ccc4c(N)nccc4c3)c2)CC1 ; Nc1nccc2cc(CNC(=O)[C@H](Cc3ccc(F)c(F)c3)NC(=O)c3cccc(-n4ccnc4)c3)ccc12] 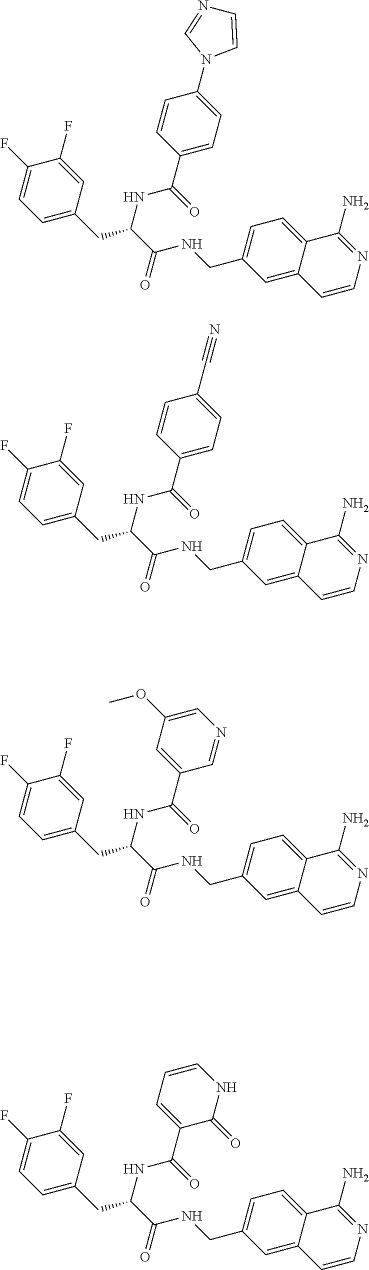 [SMILES: Nc1nccc2cc(CNC(=O)[C@H](Cc3ccc(F)c(F)c3)NC(=O)c3ccc(-n4ccnc4)cc3)ccc12 ; N#Cc1ccc(C(=O)N[C@@H](Cc2ccc(F)c(F)c2)C(=O)NCc2ccc3c(N)nccc3c2)cc1 ; COc1cncc(C(=O)N[C@@H](Cc2ccc(F)c(F)c2)C(=O)NCc2ccc3c(N)nccc3c2)c1 ; Nc1nccc2cc(CNC(=O)[C@H](Cc3ccc(F)c(F)c3)NC(=O)c3ccc[nH]c3=O)ccc12] 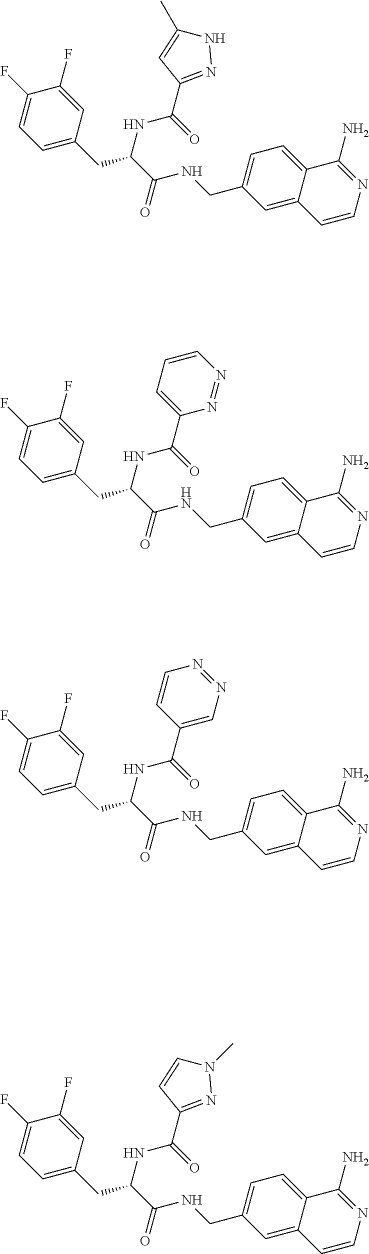 [SMILES: Cc1cc(C(=O)N[C@@H](Cc2ccc(F)c(F)c2)C(=O)NCc2ccc3c(N)nccc3c2)n[nH]1 ; Nc1nccc2cc(CNC(=O)[C@H](Cc3ccc(F)c(F)c3)NC(=O)c3cccnn3)ccc12 ; Nc1nccc2cc(CNC(=O)[C@H](Cc3ccc(F)c(F)c3)NC(=O)c3ccnnc3)ccc12 ; Cn1ccc(C(=O)N[C@@H](Cc2ccc(F)c(F)c2)C(=O)NCc2ccc3c(N)nccc3c2)n1] 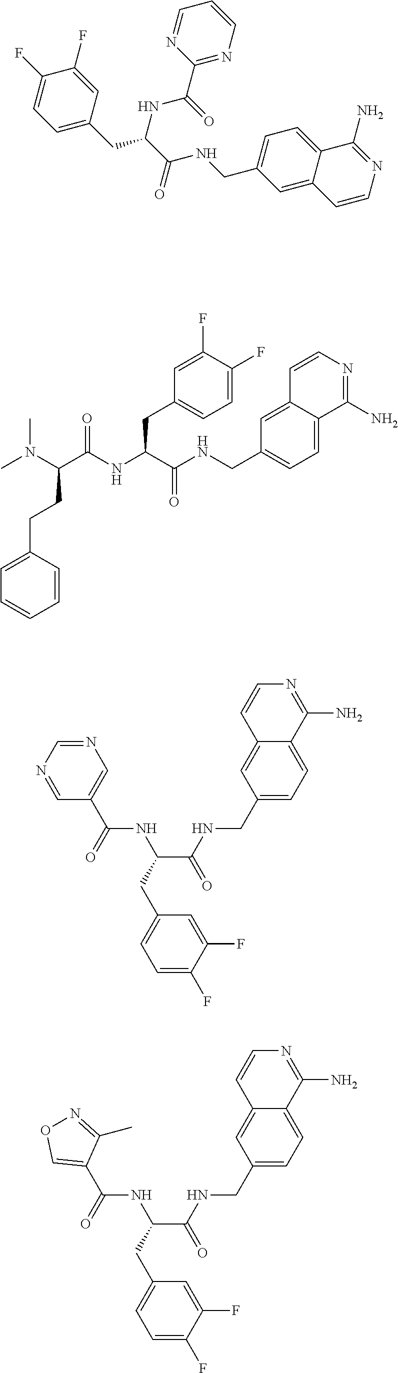 [SMILES: Nc1nccc2cc(CNC(=O)[C@H](Cc3ccc(F)c(F)c3)NC(=O)c3ncccn3)ccc12 ; CN(C)[C@H](CCc1ccccc1)C(=O)N[C@@H](Cc1ccc(F)c(F)c1)C(=O)NCc1ccc2c(N)nccc2c1 ; Nc1nccc2cc(CNC(=O)[C@H](Cc3ccc(F)c(F)c3)NC(=O)c3cncnc3)ccc12 ; Cc1nocc1C(=O)N[C@@H](Cc1ccc(F)c(F)c1)C(=O)NCc1ccc2c(N)nccc2c1] 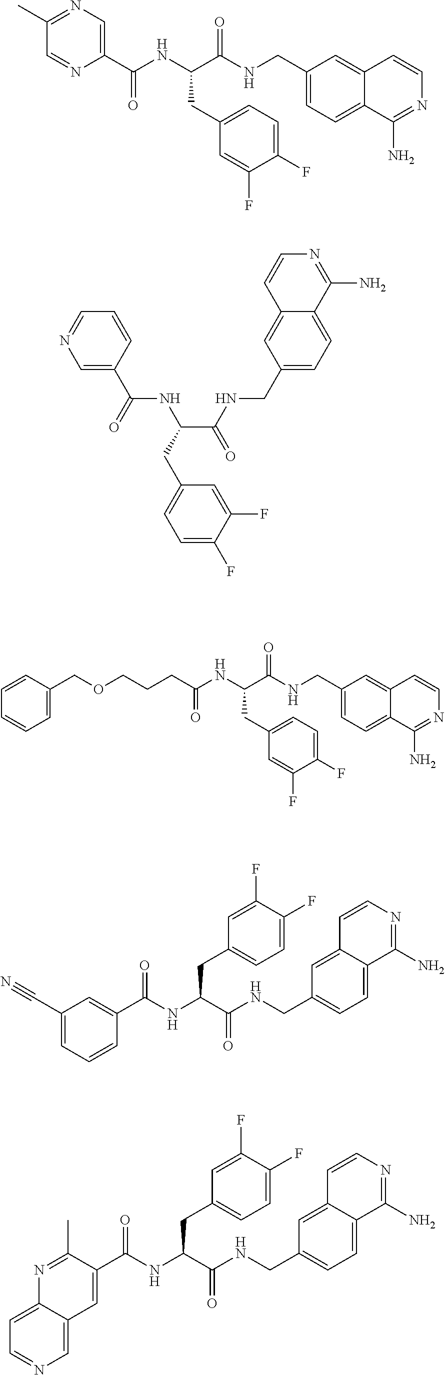 [SMILES: Cc1cnc(C(=O)N[C@@H](Cc2ccc(F)c(F)c2)C(=O)NCc2ccc3c(N)nccc3c2)cn1 ; Nc1nccc2cc(CNC(=O)[C@H](Cc3ccc(F)c(F)c3)NC(=O)c3cccnc3)ccc12 ; Nc1nccc2cc(CNC(=O)[C@H](Cc3ccc(F)c(F)c3)NC(=O)CCCOCc3ccccc3)ccc12 ; N#Cc1cccc(C(=O)N[C@@H](Cc2ccc(F)c(F)c2)C(=O)NCc2ccc3c(N)nccc3c2)c1 ; Cc1nc2ccncc2cc1C(=O)N[C@@H](Cc1ccc(F)c(F)c1)C(=O)NCc1ccc2c(N)nccc2c1] 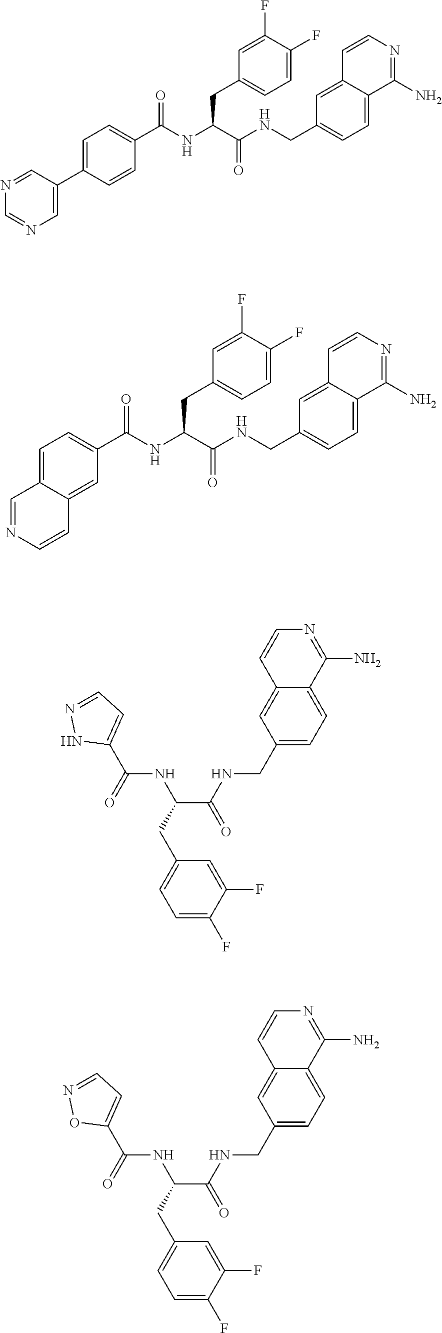 [SMILES: Nc1nccc2cc(CNC(=O)[C@H](Cc3ccc(F)c(F)c3)NC(=O)c3ccc(-c4cncnc4)cc3)ccc12 ; Nc1nccc2cc(CNC(=O)[C@H](Cc3ccc(F)c(F)c3)NC(=O)c3ccc4cnccc4c3)ccc12 ; Nc1nccc2cc(CNC(=O)[C@H](Cc3ccc(F)c(F)c3)NC(=O)c3ccn[nH]3)ccc12 ; Nc1nccc2cc(CNC(=O)[C@H](Cc3ccc(F)c(F)c3)NC(=O)c3ccno3)ccc12] 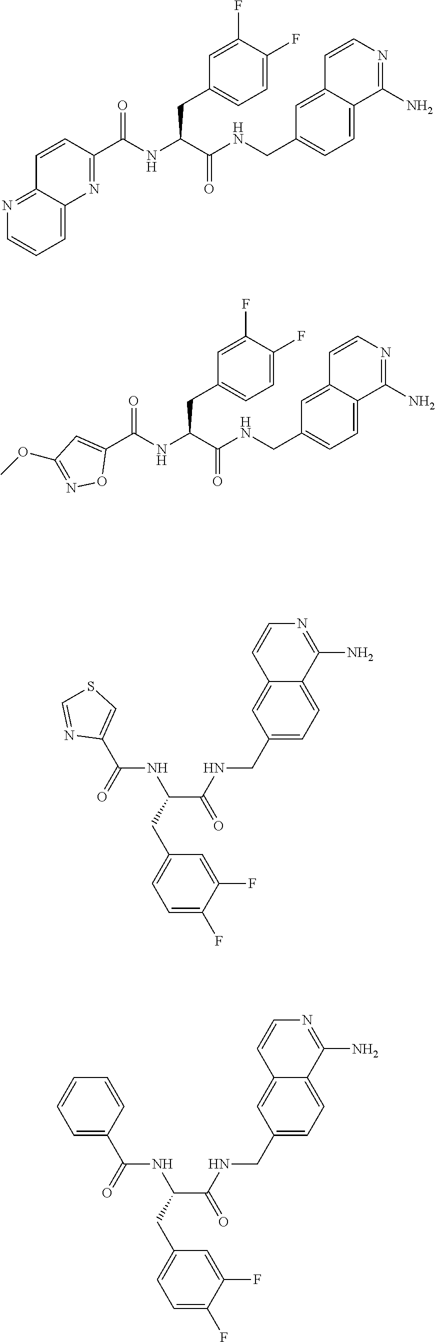 [SMILES: Nc1nccc2cc(CNC(=O)[C@H](Cc3ccc(F)c(F)c3)NC(=O)c3ccc4ncccc4n3)ccc12 ; COc1cc(C(=O)N[C@@H](Cc2ccc(F)c(F)c2)C(=O)NCc2ccc3c(N)nccc3c2)on1 ; Nc1nccc2cc(CNC(=O)[C@H](Cc3ccc(F)c(F)c3)NC(=O)c3cscn3)ccc12 ; Nc1nccc2cc(CNC(=O)[C@H](Cc3ccc(F)c(F)c3)NC(=O)c3ccccc3)ccc12] 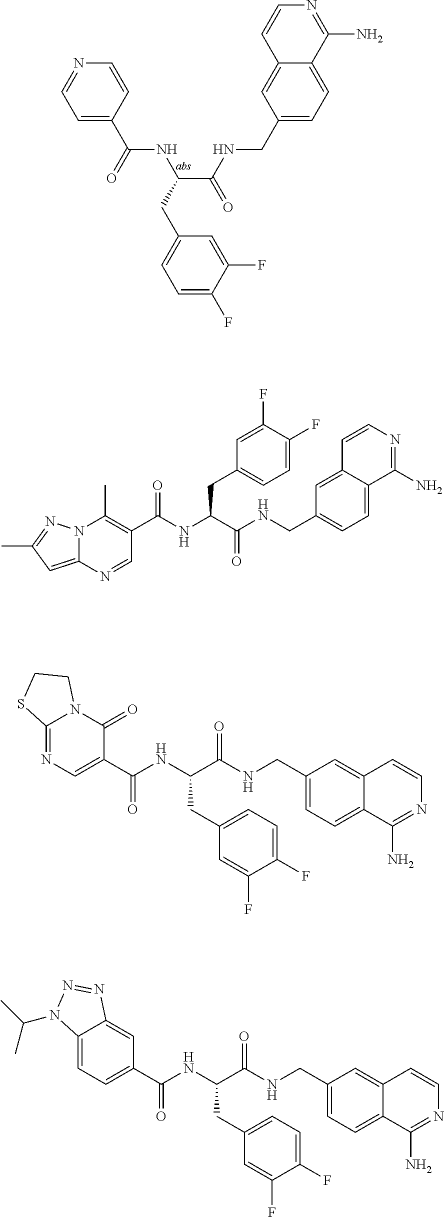 [SMILES: Nc1nccc2cc(CNC(=O)[C@H](Cc3ccc(F)c(F)c3)NC(=O)c3ccncc3)ccc12 ; Cc1cc2ncc(C(=O)N[C@@H](Cc3ccc(F)c(F)c3)C(=O)NCc3ccc4c(N)nccc4c3)c(C)n2n1 ; Nc1nccc2cc(CNC(=O)[C@H](Cc3ccc(F)c(F)c3)NC(=O)c3cnc4n(c3=O)CCS4)ccc12 ; CC(C)n1nnc2cc(C(=O)N[C@@H](Cc3ccc(F)c(F)c3)C(=O)NCc3ccc4c(N)nccc4c3)ccc21] 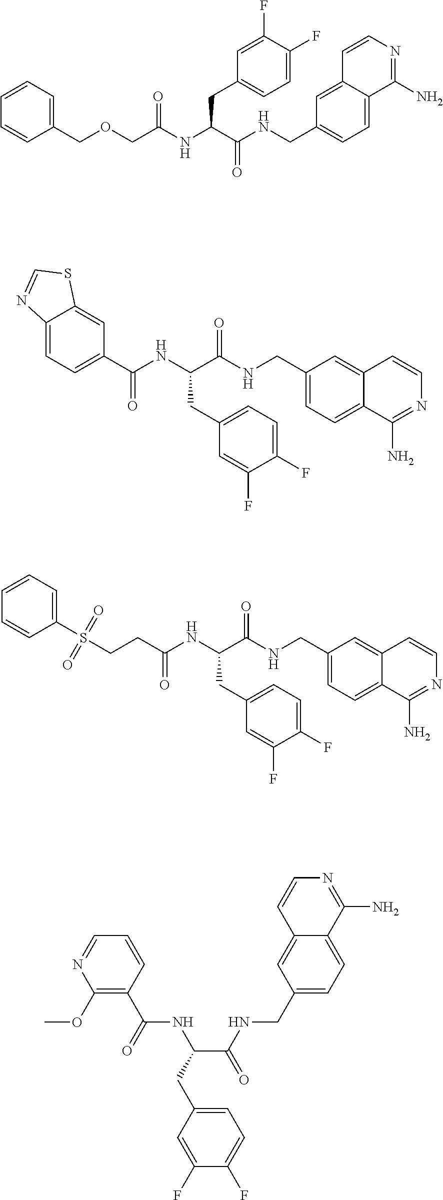 [SMILES: Nc1nccc2cc(CNC(=O)[C@H](Cc3ccc(F)c(F)c3)NC(=O)COCc3ccccc3)ccc12 ; Nc1nccc2cc(CNC(=O)[C@H](Cc3ccc(F)c(F)c3)NC(=O)c3ccc4ncsc4c3)ccc12 ; Nc1nccc2cc(CNC(=O)[C@H](Cc3ccc(F)c(F)c3)NC(=O)CCS(=O)(=O)c3ccccc3)ccc12 ; COc1ncccc1C(=O)N[C@@H](Cc1ccc(F)c(F)c1)C(=O)NCc1ccc2c(N)nccc2c1] 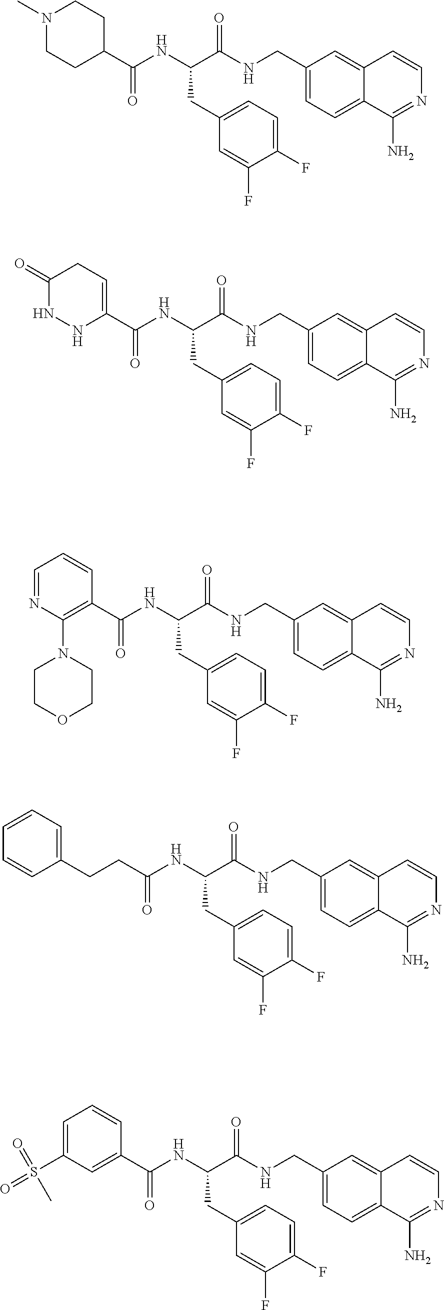 [SMILES: CN1CCC(C(=O)N[C@@H](Cc2ccc(F)c(F)c2)C(=O)NCc2ccc3c(N)nccc3c2)CC1 ; Nc1nccc2cc(CNC(=O)[C@H](Cc3ccc(F)c(F)c3)NC(=O)C3=CCC(=O)NN3)ccc12 ; Nc1nccc2cc(CNC(=O)[C@H](Cc3ccc(F)c(F)c3)NC(=O)c3cccnc3N3CCOCC3)ccc12 ; Nc1nccc2cc(CNC(=O)[C@H](Cc3ccc(F)c(F)c3)NC(=O)CCc3ccccc3)ccc12 ; CS(=O)(=O)c1cccc(C(=O)N[C@@H](Cc2ccc(F)c(F)c2)C(=O)NCc2ccc3c(N)nccc3c2)c1] 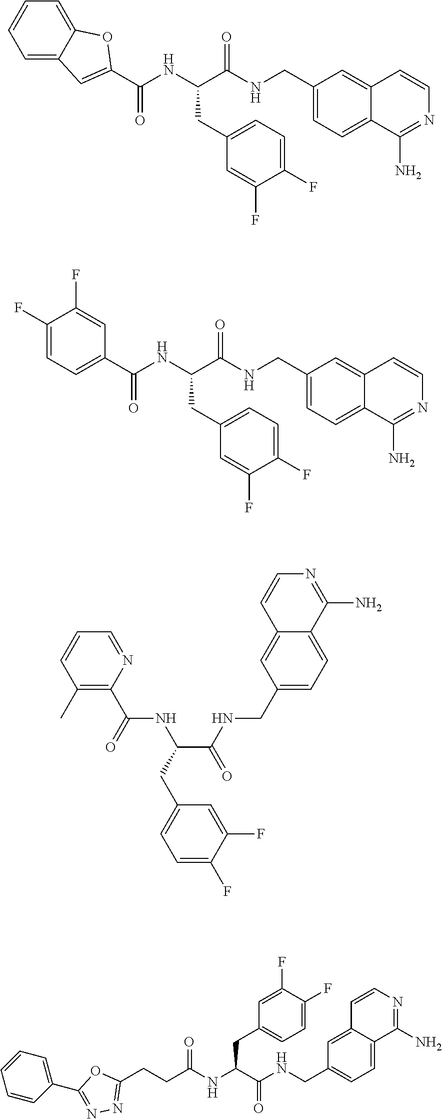 [SMILES: Nc1nccc2cc(CNC(=O)[C@H](Cc3ccc(F)c(F)c3)NC(=O)c3cc4ccccc4o3)ccc12 ; Nc1nccc2cc(CNC(=O)[C@H](Cc3ccc(F)c(F)c3)NC(=O)c3ccc(F)c(F)c3)ccc12 ; Cc1cccnc1C(=O)N[C@@H](Cc1ccc(F)c(F)c1)C(=O)NCc1ccc2c(N)nccc2c1 ; Nc1nccc2cc(CNC(=O)[C@H](Cc3ccc(F)c(F)c3)NC(=O)CCc3nnc(-c4ccccc4)o3)ccc12] 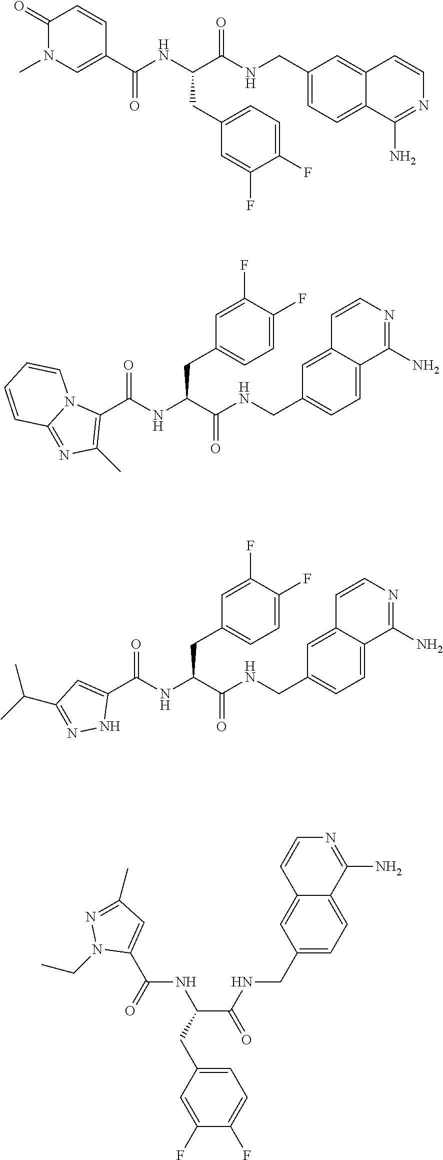 [SMILES: Cn1cc(C(=O)N[C@@H](Cc2ccc(F)c(F)c2)C(=O)NCc2ccc3c(N)nccc3c2)ccc1=O ; Cc1nc2ccccn2c1C(=O)N[C@@H](Cc1ccc(F)c(F)c1)C(=O)NCc1ccc2c(N)nccc2c1 ; CC(C)c1cc(C(=O)N[C@@H](Cc2ccc(F)c(F)c2)C(=O)NCc2ccc3c(N)nccc3c2)[nH]n1 ; CCn1nc(C)cc1C(=O)N[C@@H](Cc1ccc(F)c(F)c1)C(=O)NCc1ccc2c(N)nccc2c1] 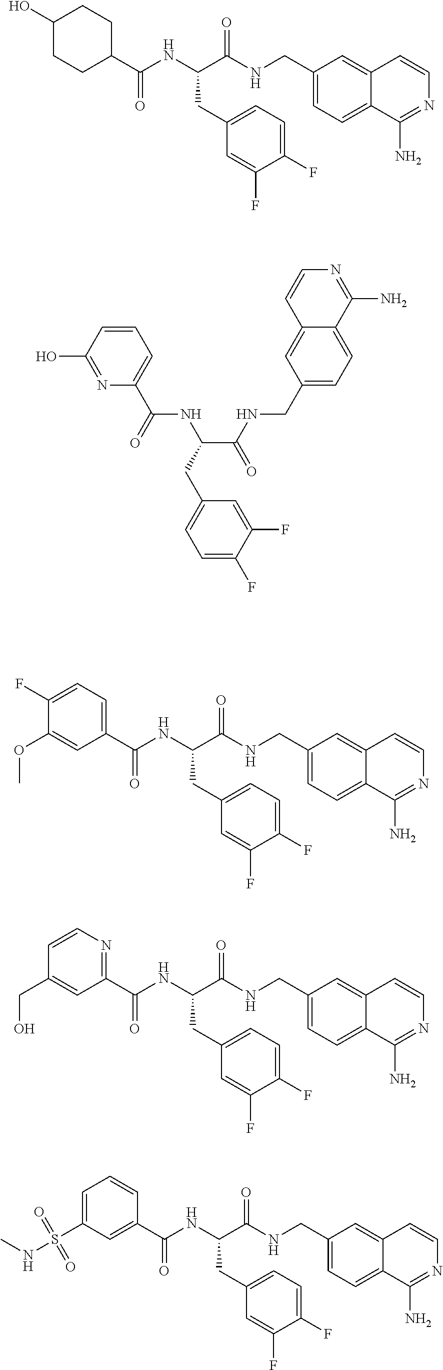 [SMILES: Nc1nccc2cc(CNC(=O)[C@H](Cc3ccc(F)c(F)c3)NC(=O)C3CCC(O)CC3)ccc12 ; Nc1nccc2cc(CNC(=O)[C@H](Cc3ccc(F)c(F)c3)NC(=O)c3cccc(O)n3)ccc12 ; COc1cc(C(=O)N[C@@H](Cc2ccc(F)c(F)c2)C(=O)NCc2ccc3c(N)nccc3c2)ccc1F ; Nc1nccc2cc(CNC(=O)[C@H](Cc3ccc(F)c(F)c3)NC(=O)c3cc(CO)ccn3)ccc12 ; CNS(=O)(=O)c1cccc(C(=O)N[C@@H](Cc2ccc(F)c(F)c2)C(=O)NCc2ccc3c(N)nccc3c2)c1] 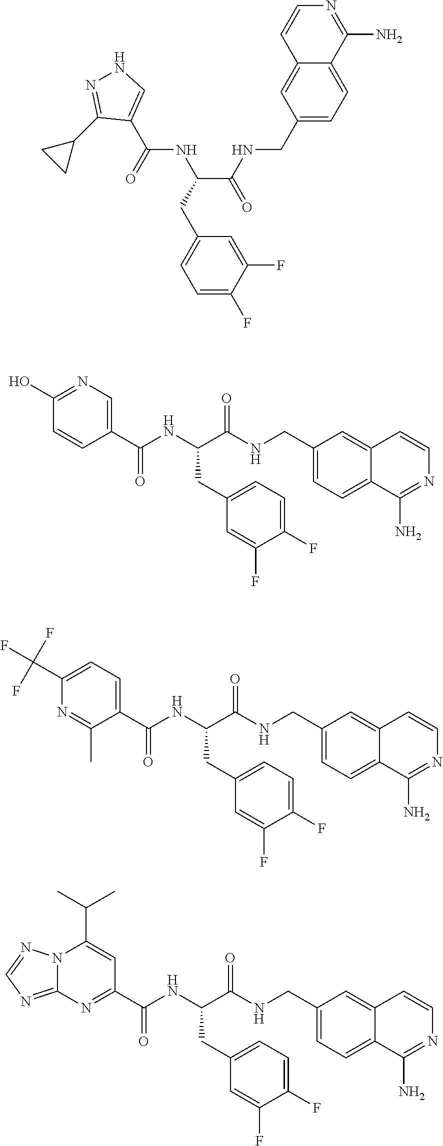 [SMILES: Nc1nccc2cc(CNC(=O)[C@H](Cc3ccc(F)c(F)c3)NC(=O)c3c[nH]nc3C3CC3)ccc12 ; Nc1nccc2cc(CNC(=O)[C@H](Cc3ccc(F)c(F)c3)NC(=O)c3ccc(O)nc3)ccc12 ; Cc1nc(C(F)(F)F)ccc1C(=O)N[C@@H](Cc1ccc(F)c(F)c1)C(=O)NCc1ccc2c(N)nccc2c1 ; CC(C)c1cc(C(=O)N[C@@H](Cc2ccc(F)c(F)c2)C(=O)NCc2ccc3c(N)nccc3c2)nc2ncnn12] 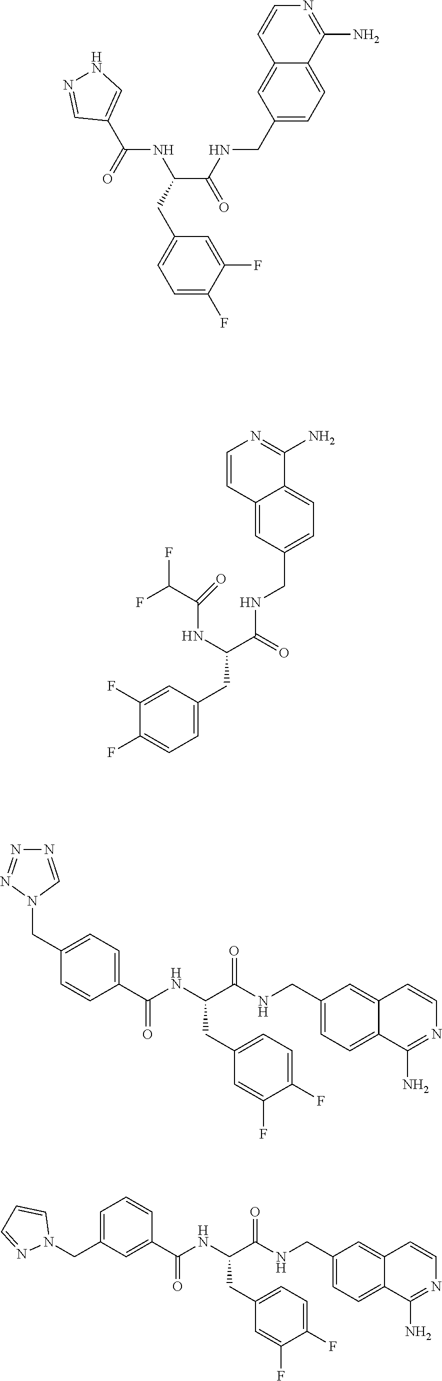 [SMILES: Nc1nccc2cc(CNC(=O)[C@H](Cc3ccc(F)c(F)c3)NC(=O)c3cn[nH]c3)ccc12 ; Nc1nccc2cc(CNC(=O)[C@H](Cc3ccc(F)c(F)c3)NC(=O)C(F)F)ccc12 ; Nc1nccc2cc(CNC(=O)[C@H](Cc3ccc(F)c(F)c3)NC(=O)c3ccc(Cn4cnnn4)cc3)ccc12 ; Nc1nccc2cc(CNC(=O)[C@H](Cc3ccc(F)c(F)c3)NC(=O)c3cccc(Cn4cccn4)c3)ccc12] 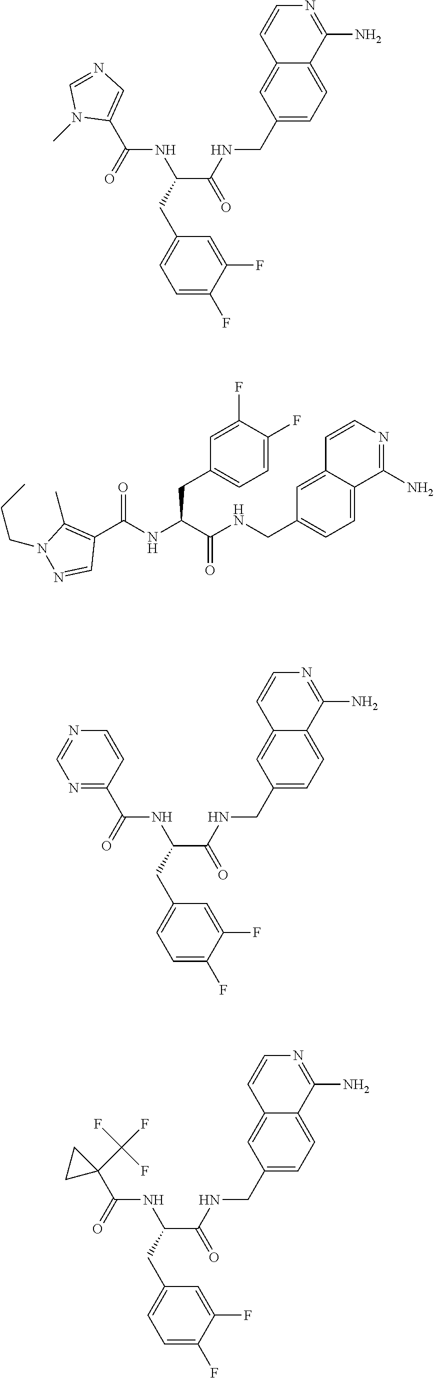 [SMILES: Cn1cncc1C(=O)N[C@@H](Cc1ccc(F)c(F)c1)C(=O)NCc1ccc2c(N)nccc2c1 ; CCCn1ncc(C(=O)N[C@@H](Cc2ccc(F)c(F)c2)C(=O)NCc2ccc3c(N)nccc3c2)c1C ; Nc1nccc2cc(CNC(=O)[C@H](Cc3ccc(F)c(F)c3)NC(=O)c3ccncn3)ccc12 ; Nc1nccc2cc(CNC(=O)[C@H](Cc3ccc(F)c(F)c3)NC(=O)C3(C(F)(F)F)CC3)ccc12] 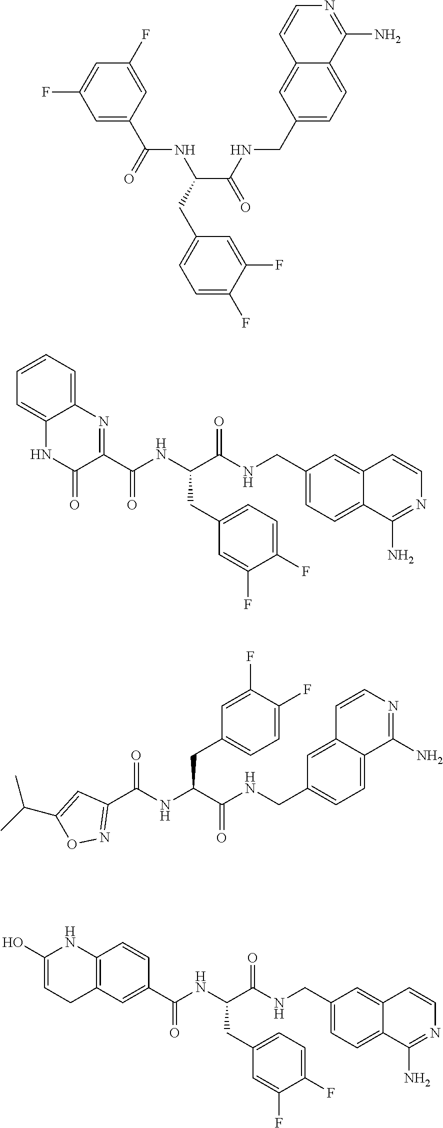 [SMILES: Nc1nccc2cc(CNC(=O)[C@H](Cc3ccc(F)c(F)c3)NC(=O)c3cc(F)cc(F)c3)ccc12 ; Nc1nccc2cc(CNC(=O)[C@H](Cc3ccc(F)c(F)c3)NC(=O)c3nc4ccccc4[nH]c3=O)ccc12 ; CC(C)c1cc(C(=O)N[C@@H](Cc2ccc(F)c(F)c2)C(=O)NCc2ccc3c(N)nccc3c2)no1 ; Nc1nccc2cc(CNC(=O)[C@H](Cc3ccc(F)c(F)c3)NC(=O)c3ccc4c(c3)CC=C(O)N4)ccc12] 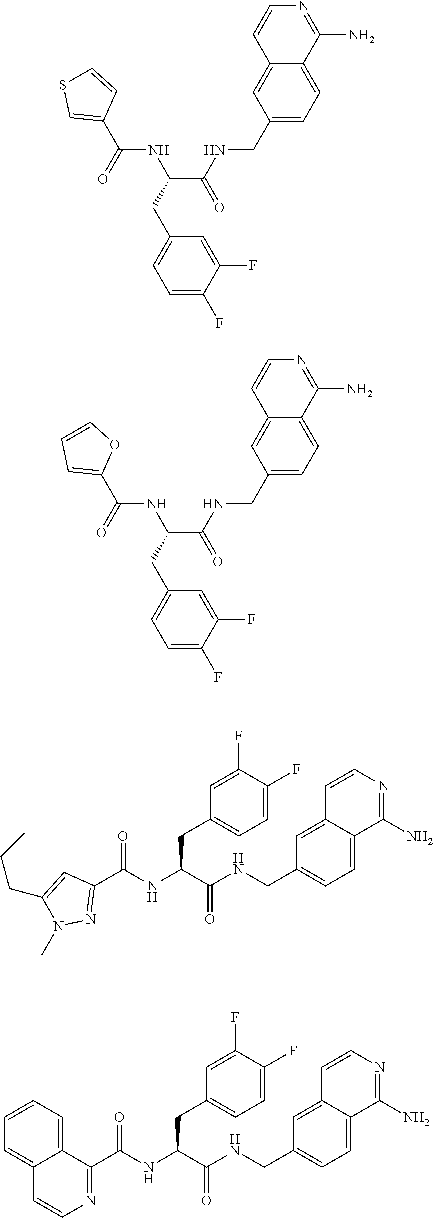 [SMILES: Nc1nccc2cc(CNC(=O)[C@H](Cc3ccc(F)c(F)c3)NC(=O)c3ccsc3)ccc12 ; Nc1nccc2cc(CNC(=O)[C@H](Cc3ccc(F)c(F)c3)NC(=O)c3ccco3)ccc12 ; CCCc1cc(C(=O)N[C@@H](Cc2ccc(F)c(F)c2)C(=O)NCc2ccc3c(N)nccc3c2)nn1C ; Nc1nccc2cc(CNC(=O)[C@H](Cc3ccc(F)c(F)c3)NC(=O)c3nccc4ccccc34)ccc12] 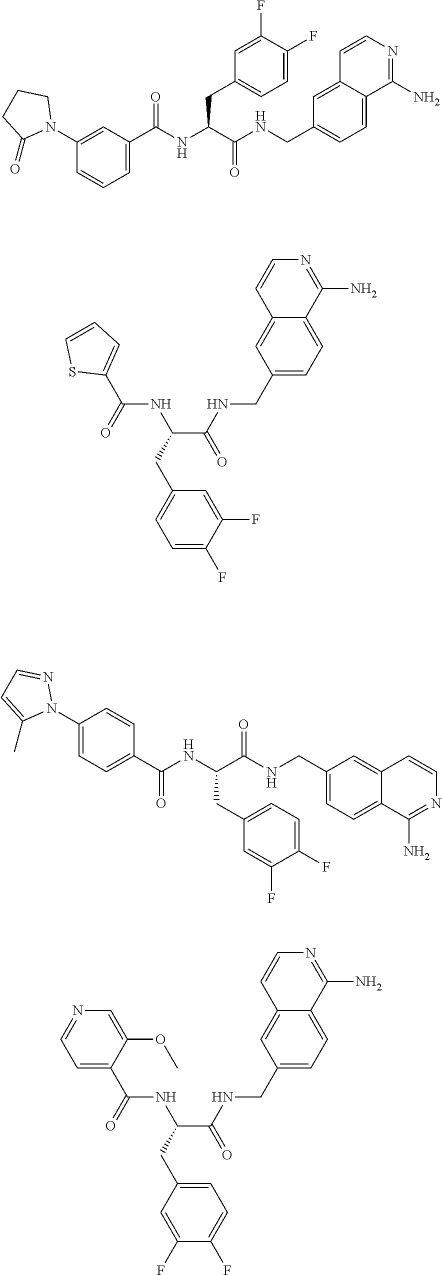 [SMILES: Nc1nccc2cc(CNC(=O)[C@H](Cc3ccc(F)c(F)c3)NC(=O)c3cccc(N4CCCC4=O)c3)ccc12 ; Nc1nccc2cc(CNC(=O)[C@H](Cc3ccc(F)c(F)c3)NC(=O)c3cccs3)ccc12 ; Cc1ccnn1-c1ccc(C(=O)N[C@@H](Cc2ccc(F)c(F)c2)C(=O)NCc2ccc3c(N)nccc3c2)cc1 ; COc1cnccc1C(=O)N[C@@H](Cc1ccc(F)c(F)c1)C(=O)NCc1ccc2c(N)nccc2c1] 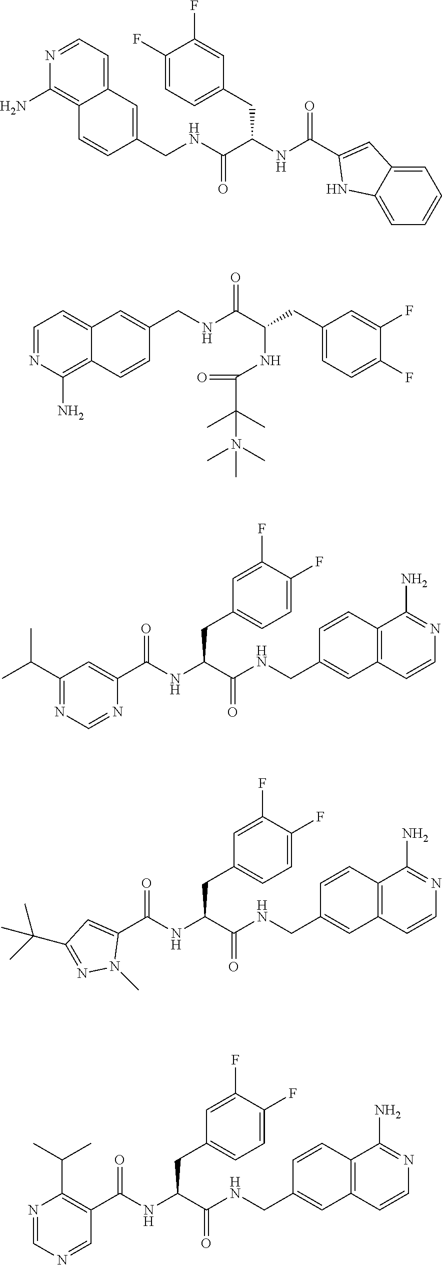 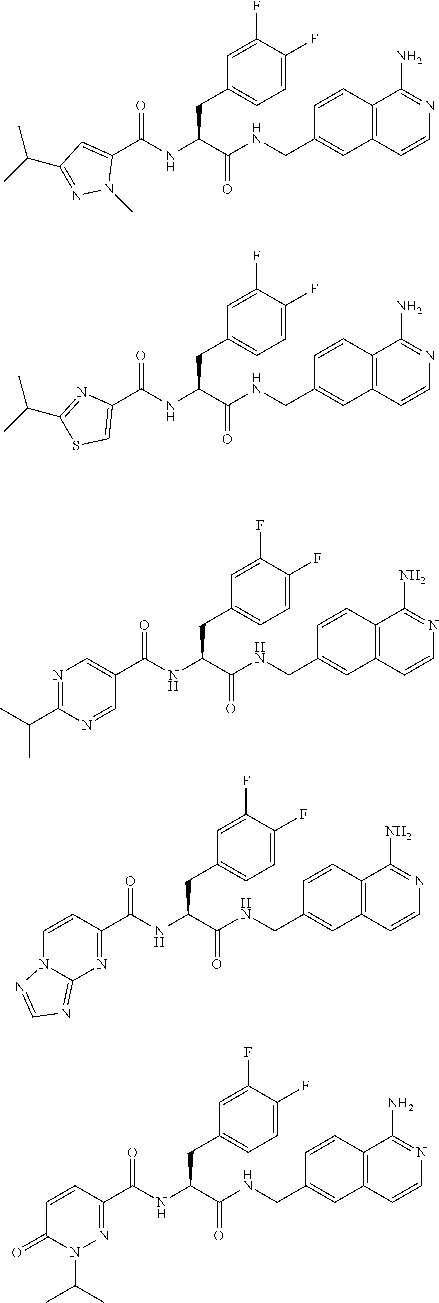 [SMILES: CC(C)c1cc(C(=O)N[C@@H](Cc2ccc(F)c(F)c2)C(=O)NCc2ccc3c(N)nccc3c2)n(C)n1 ; CC(C)c1nc(C(=O)N[C@@H](Cc2ccc(F)c(F)c2)C(=O)NCc2ccc3c(N)nccc3c2)cs1 ; CC(C)c1ncc(C(=O)N[C@@H](Cc2ccc(F)c(F)c2)C(=O)NCc2ccc3c(N)nccc3c2)cn1 ; Nc1nccc2cc(CNC(=O)[C@H](Cc3ccc(F)c(F)c3)NC(=O)c3ccn4ncnc4n3)ccc12 ; CC(C)n1nc(C(=O)N[C@@H](Cc2ccc(F)c(F)c2)C(=O)NCc2ccc3c(N)nccc3c2)ccc1=O] 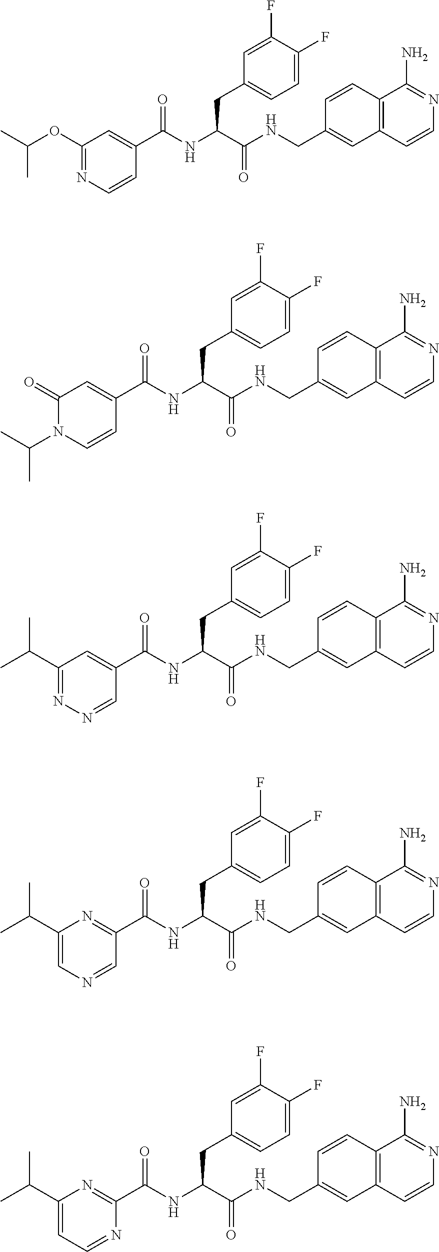 [SMILES: CC(C)Oc1cc(C(=O)N[C@@H](Cc2ccc(F)c(F)c2)C(=O)NCc2ccc3c(N)nccc3c2)ccn1 ; CC(C)n1ccc(C(=O)N[C@@H](Cc2ccc(F)c(F)c2)C(=O)NCc2ccc3c(N)nccc3c2)cc1=O ; CC(C)c1cc(C(=O)N[C@@H](Cc2ccc(F)c(F)c2)C(=O)NCc2ccc3c(N)nccc3c2)cnn1 ; CC(C)c1cncc(C(=O)N[C@@H](Cc2ccc(F)c(F)c2)C(=O)NCc2ccc3c(N)nccc3c2)n1 ; CC(C)c1ccnc(C(=O)N[C@@H](Cc2ccc(F)c(F)c2)C(=O)NCc2ccc3c(N)nccc3c2)n1] 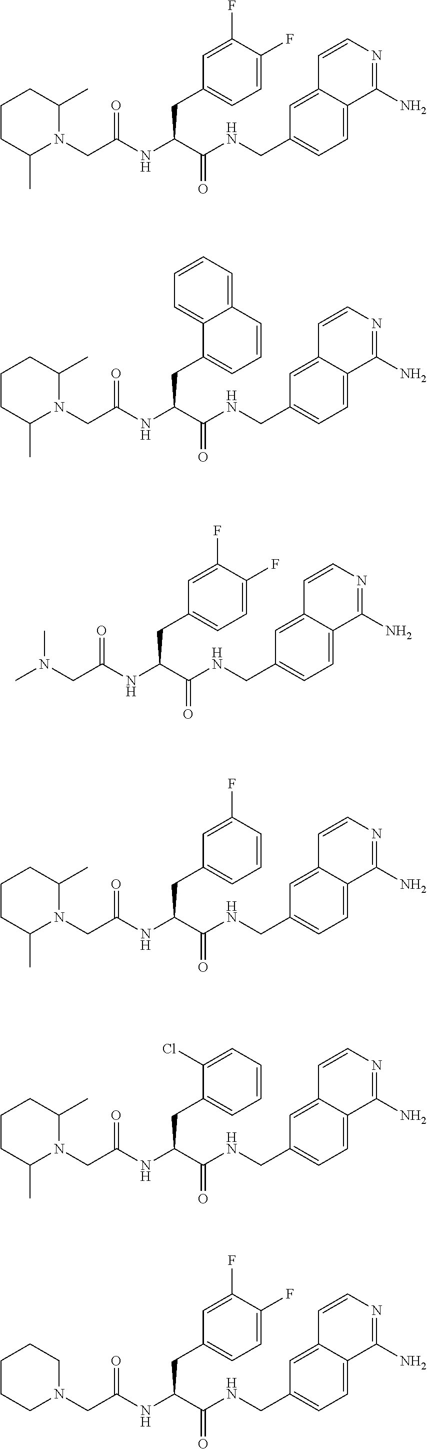 [SMILES: CC1CCCC(C)N1CC(=O)N[C@@H](Cc1ccc(F)c(F)c1)C(=O)NCc1ccc2c(N)nccc2c1 ; CC1CCCC(C)N1CC(=O)N[C@@H](Cc1cccc2ccccc12)C(=O)NCc1ccc2c(N)nccc2c1 ; CN(C)CC(=O)N[C@@H](Cc1ccc(F)c(F)c1)C(=O)NCc1ccc2c(N)nccc2c1 ; CC1CCCC(C)N1CC(=O)N[C@@H](Cc1cccc(F)c1)C(=O)NCc1ccc2c(N)nccc2c1 ; CC1CCCC(C)N1CC(=O)N[C@@H](Cc1ccccc1Cl)C(=O)NCc1ccc2c(N)nccc2c1 ; Nc1nccc2cc(CNC(=O)[C@H](Cc3ccc(F)c(F)c3)NC(=O)CN3CCCCC3)ccc12] 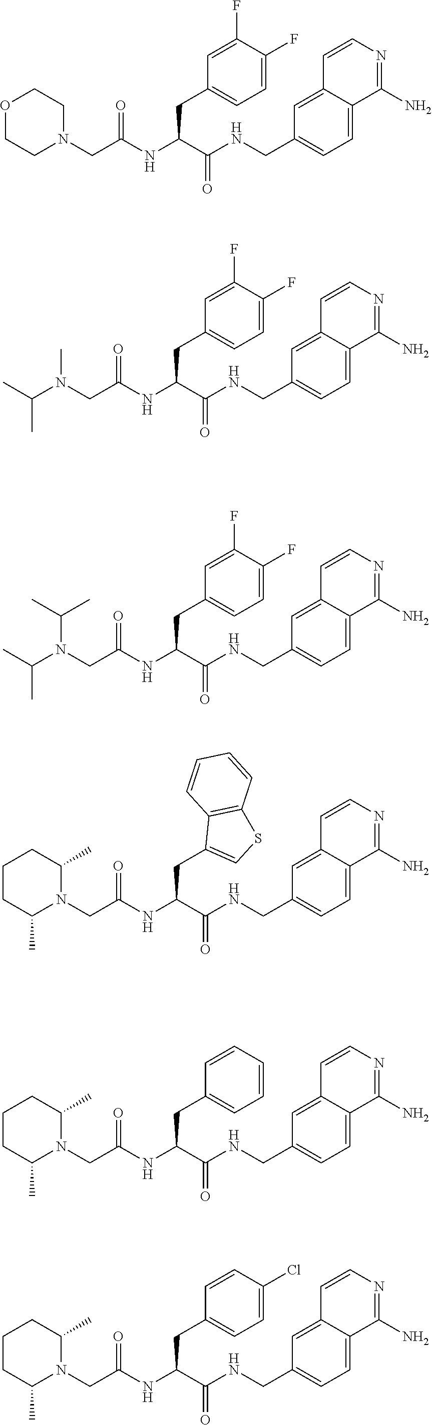 [SMILES: Nc1nccc2cc(CNC(=O)[C@H](Cc3ccc(F)c(F)c3)NC(=O)CN3CCOCC3)ccc12 ; CC(C)N(C)CC(=O)N[C@@H](Cc1ccc(F)c(F)c1)C(=O)NCc1ccc2c(N)nccc2c1 ; CC(C)N(CC(=O)N[C@@H](Cc1ccc(F)c(F)c1)C(=O)NCc1ccc2c(N)nccc2c1)C(C)C ; C[C@@H]1CCC[C@H](C)N1CC(=O)N[C@@H](Cc1csc2ccccc12)C(=O)NCc1ccc2c(N)nccc2c1 ; C[C@@H]1CCC[C@H](C)N1CC(=O)N[C@@H](Cc1ccccc1)C(=O)NCc1ccc2c(N)nccc2c1 ; C[C@@H]1CCC[C@H](C)N1CC(=O)N[C@@H](Cc1ccc(Cl)cc1)C(=O)NCc1ccc2c(N)nccc2c1] 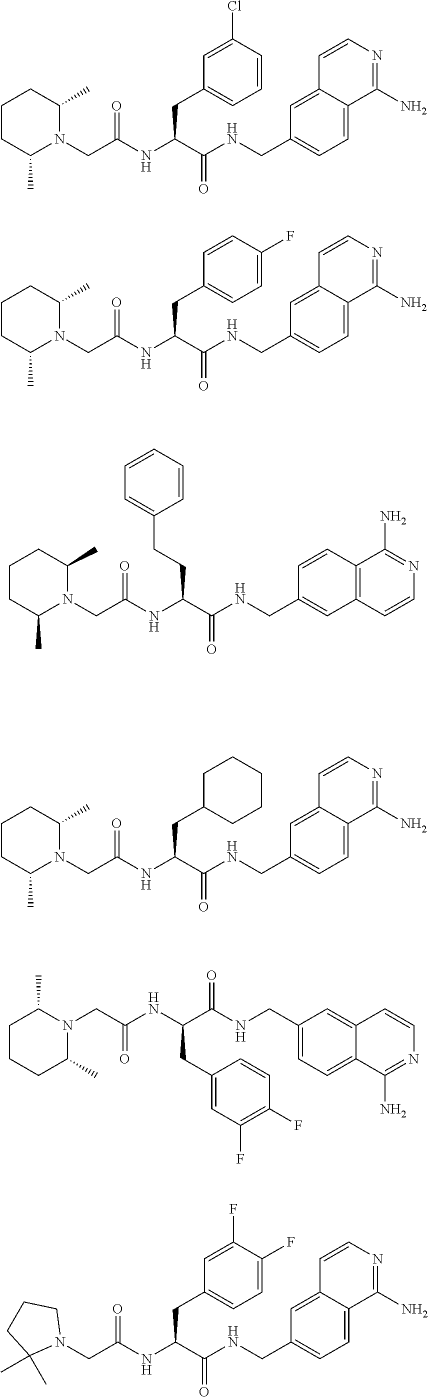 [SMILES: C[C@@H]1CCC[C@H](C)N1CC(=O)N[C@@H](Cc1cccc(Cl)c1)C(=O)NCc1ccc2c(N)nccc2c1 ; C[C@@H]1CCC[C@H](C)N1CC(=O)N[C@@H](Cc1ccc(F)cc1)C(=O)NCc1ccc2c(N)nccc2c1 ; C[C@@H]1CCC[C@H](C)N1CC(=O)N[C@@H](CCc1ccccc1)C(=O)NCc1ccc2c(N)nccc2c1 ; C[C@@H]1CCC[C@H](C)N1CC(=O)N[C@@H](CC1CCCCC1)C(=O)NCc1ccc2c(N)nccc2c1 ; C[C@@H]1CCC[C@H](C)N1CC(=O)N[C@H](Cc1ccc(F)c(F)c1)C(=O)NCc1ccc2c(N)nccc2c1 ; CC1(C)CCCN1CC(=O)N[C@@H](Cc1ccc(F)c(F)c1)C(=O)NCc1ccc2c(N)nccc2c1] 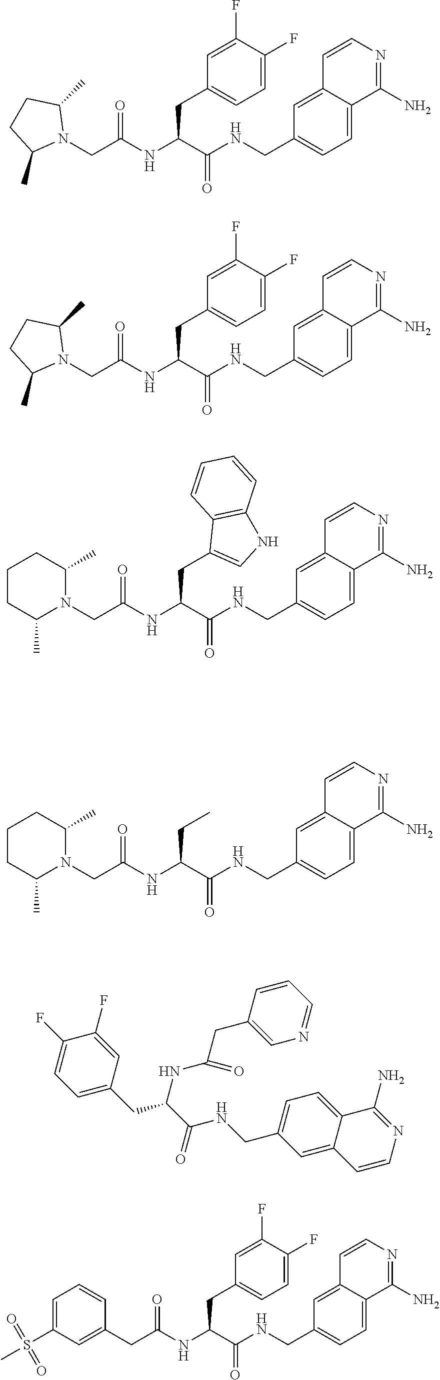 [SMILES: C[C@H]1CC[C@H](C)N1CC(=O)N[C@@H](Cc1ccc(F)c(F)c1)C(=O)NCc1ccc2c(N)nccc2c1 ; C[C@@H]1CC[C@H](C)N1CC(=O)N[C@@H](Cc1ccc(F)c(F)c1)C(=O)NCc1ccc2c(N)nccc2c1 ; C[C@@H]1CCC[C@H](C)N1CC(=O)N[C@@H](Cc1c[nH]c2ccccc12)C(=O)NCc1ccc2c(N)nccc2c1 ; CC[C@H](NC(=O)CN1[C@H](C)CCC[C@@H]1C)C(=O)NCc1ccc2c(N)nccc2c1 ; Nc1nccc2cc(CNC(=O)[C@H](Cc3ccc(F)c(F)c3)NC(=O)Cc3cccnc3)ccc12 ; CS(=O)(=O)c1cccc(CC(=O)N[C@@H](Cc2ccc(F)c(F)c2)C(=O)NCc2ccc3c(N)nccc3c2)c1] 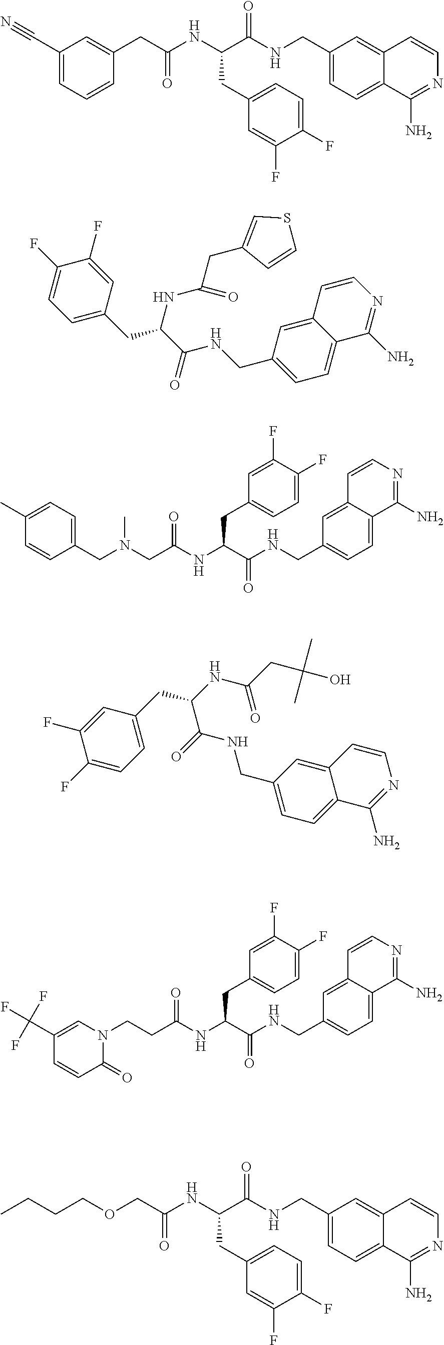 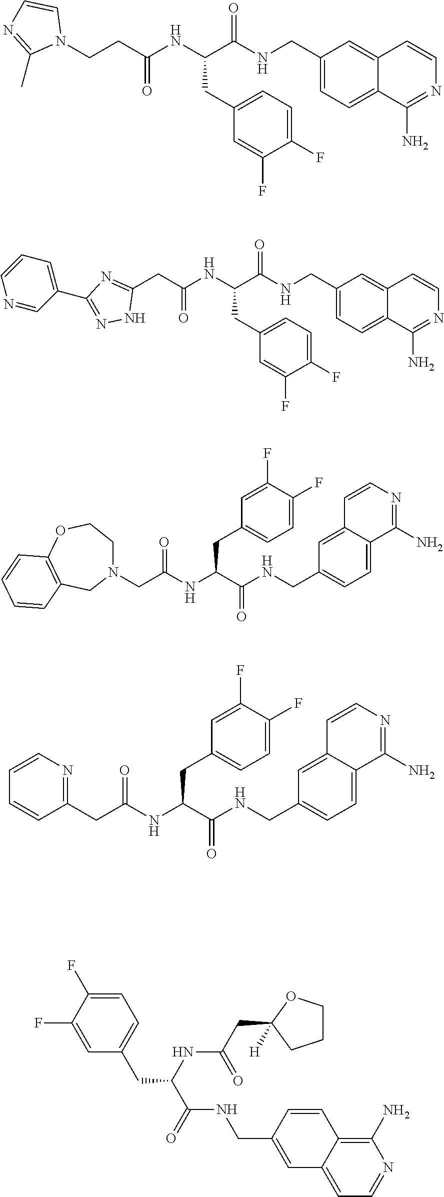 [SMILES: Cc1nccn1CCC(=O)N[C@@H](Cc1ccc(F)c(F)c1)C(=O)NCc1ccc2c(N)nccc2c1 ; Nc1nccc2cc(CNC(=O)[C@H](Cc3ccc(F)c(F)c3)NC(=O)Cc3nc(-c4cccnc4)n[nH]3)ccc12 ; Nc1nccc2cc(CNC(=O)[C@H](Cc3ccc(F)c(F)c3)NC(=O)CN3CCOc4ccccc4C3)ccc12 ; Nc1nccc2cc(CNC(=O)[C@H](Cc3ccc(F)c(F)c3)NC(=O)Cc3ccccn3)ccc12 ; Nc1nccc2cc(CNC(=O)[C@H](Cc3ccc(F)c(F)c3)NC(=O)C[C@@H]3CCCO3)ccc12] 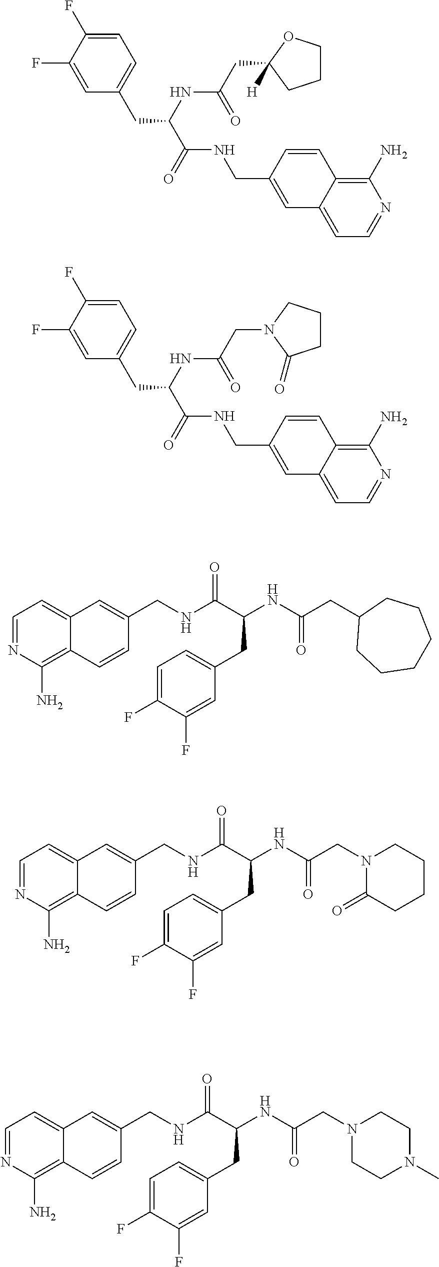 [SMILES: Nc1nccc2cc(CNC(=O)[C@H](Cc3ccc(F)c(F)c3)NC(=O)C[C@H]3CCCO3)ccc12 ; Nc1nccc2cc(CNC(=O)[C@H](Cc3ccc(F)c(F)c3)NC(=O)CN3CCCC3=O)ccc12 ; Nc1nccc2cc(CNC(=O)[C@H](Cc3ccc(F)c(F)c3)NC(=O)CC3CCCCCC3)ccc12 ; Nc1nccc2cc(CNC(=O)[C@H](Cc3ccc(F)c(F)c3)NC(=O)CN3CCCCC3=O)ccc12 ; CN1CCN(CC(=O)N[C@@H](Cc2ccc(F)c(F)c2)C(=O)NCc2ccc3c(N)nccc3c2)CC1] 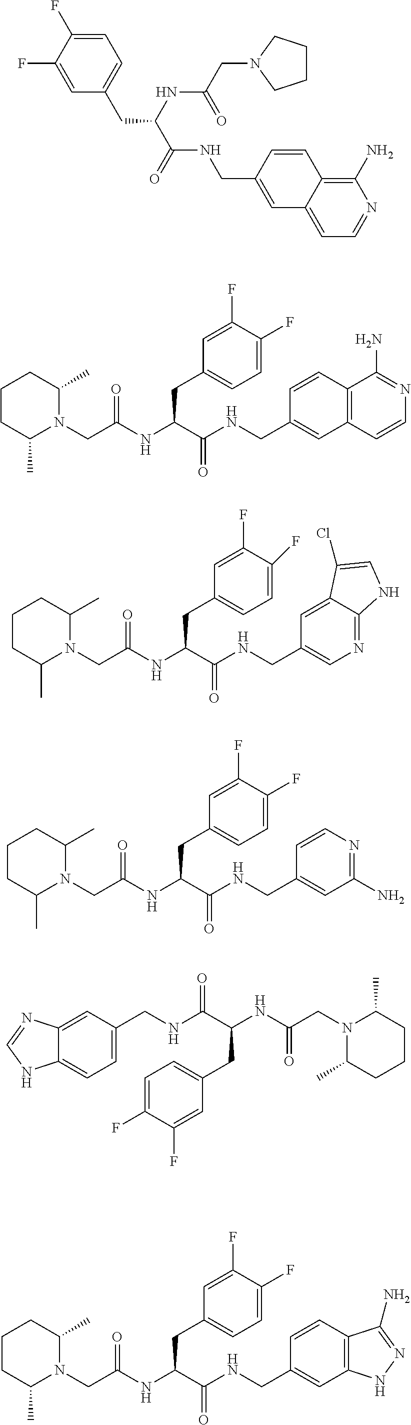 [SMILES: Nc1nccc2cc(CNC(=O)[C@H](Cc3ccc(F)c(F)c3)NC(=O)CN3CCCC3)ccc12 ; C[C@@H]1CCC[C@H](C)N1CC(=O)N[C@@H](Cc1ccc(F)c(F)c1)C(=O)NCc1ccc2c(N)nccc2c1 ; CC1CCCC(C)N1CC(=O)N[C@@H](Cc1ccc(F)c(F)c1)C(=O)NCc1cnc2[nH]cc(Cl)c2c1 ; CC1CCCC(C)N1CC(=O)N[C@@H](Cc1ccc(F)c(F)c1)C(=O)NCc1ccnc(N)c1 ; C[C@@H]1CCC[C@H](C)N1CC(=O)N[C@@H](Cc1ccc(F)c(F)c1)C(=O)NCc1ccc2[nH]cnc2c1 ; C[C@@H]1CCC[C@H](C)N1CC(=O)N[C@@H](Cc1ccc(F)c(F)c1)C(=O)NCc1ccc2c(N)n[nH]c2c1] 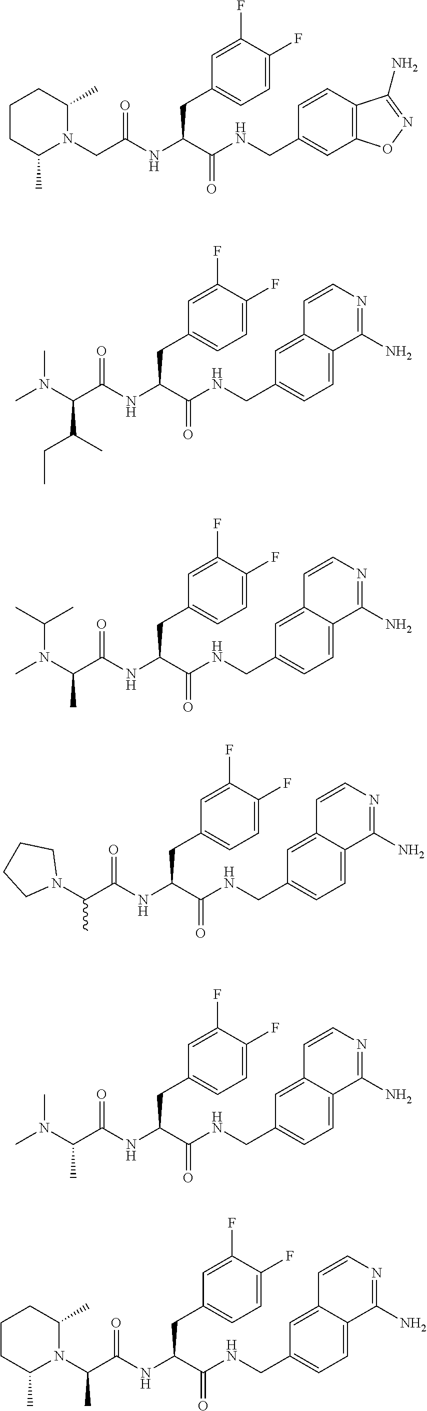 [SMILES: C[C@@H]1CCC[C@H](C)N1CC(=O)N[C@@H](Cc1ccc(F)c(F)c1)C(=O)NCc1ccc2c(N)noc2c1 ; CCC(C)[C@H](C(=O)N[C@@H](Cc1ccc(F)c(F)c1)C(=O)NCc1ccc2c(N)nccc2c1)N(C)C ; CC(C)N(C)[C@H](C)C(=O)N[C@@H](Cc1ccc(F)c(F)c1)C(=O)NCc1ccc2c(N)nccc2c1 ; CC(C(=O)N[C@@H](Cc1ccc(F)c(F)c1)C(=O)NCc1ccc2c(N)nccc2c1)N1CCCC1 ; C[C@@H](C(=O)N[C@@H](Cc1ccc(F)c(F)c1)C(=O)NCc1ccc2c(N)nccc2c1)N(C)C ; C[C@@H]1CCC[C@H](C)N1[C@H](C)C(=O)N[C@@H](Cc1ccc(F)c(F)c1)C(=O)NCc1ccc2c(N)nccc2c1] 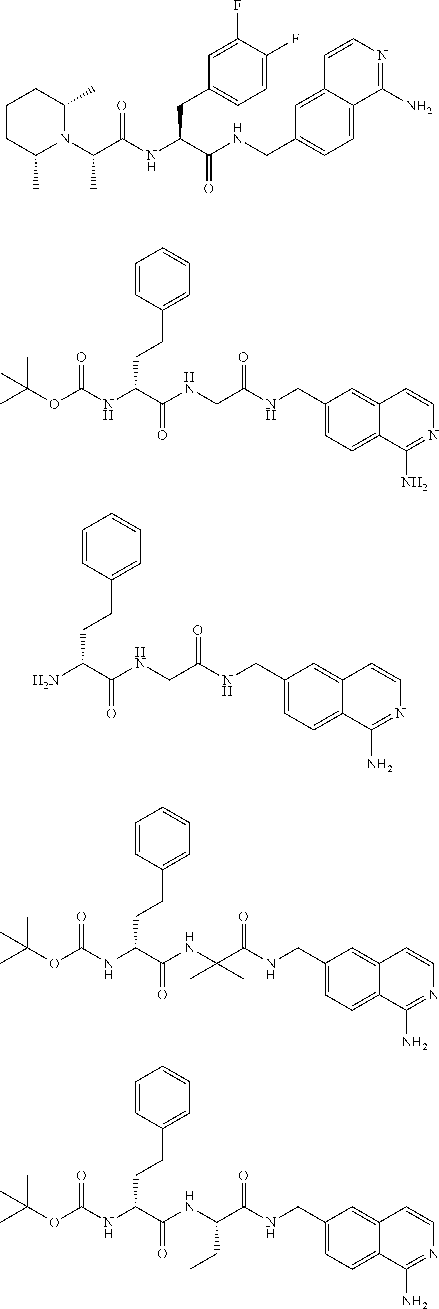 [SMILES: C[C@@H]1CCC[C@H](C)N1[C@@H](C)C(=O)N[C@@H](Cc1ccc(F)c(F)c1)C(=O)NCc1ccc2c(N)nccc2c1 ; CC(C)(C)OC(=O)N[C@H](CCc1ccccc1)C(=O)NCC(=O)NCc1ccc2c(N)nccc2c1 ; Nc1nccc2cc(CNC(=O)CNC(=O)[C@H](N)CCc3ccccc3)ccc12 ; CC(C)(C)OC(=O)N[C@H](CCc1ccccc1)C(=O)NC(C)(C)C(=O)NCc1ccc2c(N)nccc2c1 ; CC[C@H](NC(=O)[C@@H](CCc1ccccc1)NC(=O)OC(C)(C)C)C(=O)NCc1ccc2c(N)nccc2c1] 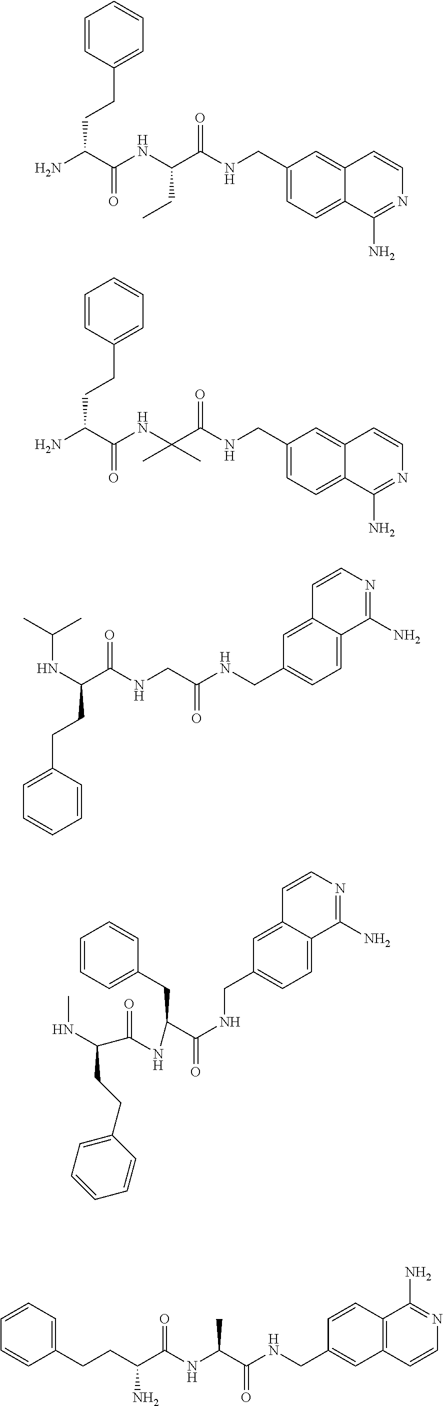 [SMILES: CC[C@H](NC(=O)[C@H](N)CCc1ccccc1)C(=O)NCc1ccc2c(N)nccc2c1 ; CC(C)(NC(=O)[C@H](N)CCc1ccccc1)C(=O)NCc1ccc2c(N)nccc2c1 ; CC(C)N[C@H](CCc1ccccc1)C(=O)NCC(=O)NCc1ccc2c(N)nccc2c1 ; CN[C@H](CCc1ccccc1)C(=O)N[C@@H](Cc1ccccc1)C(=O)NCc1ccc2c(N)nccc2c1 ; C[C@H](NC(=O)[C@H](N)CCc1ccccc1)C(=O)NCc1ccc2c(N)nccc2c1] 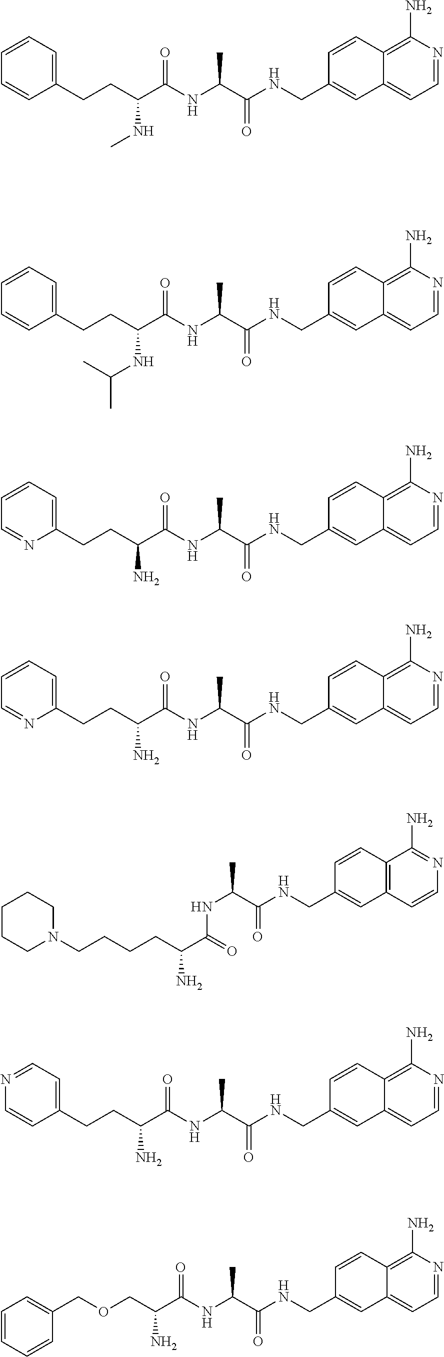 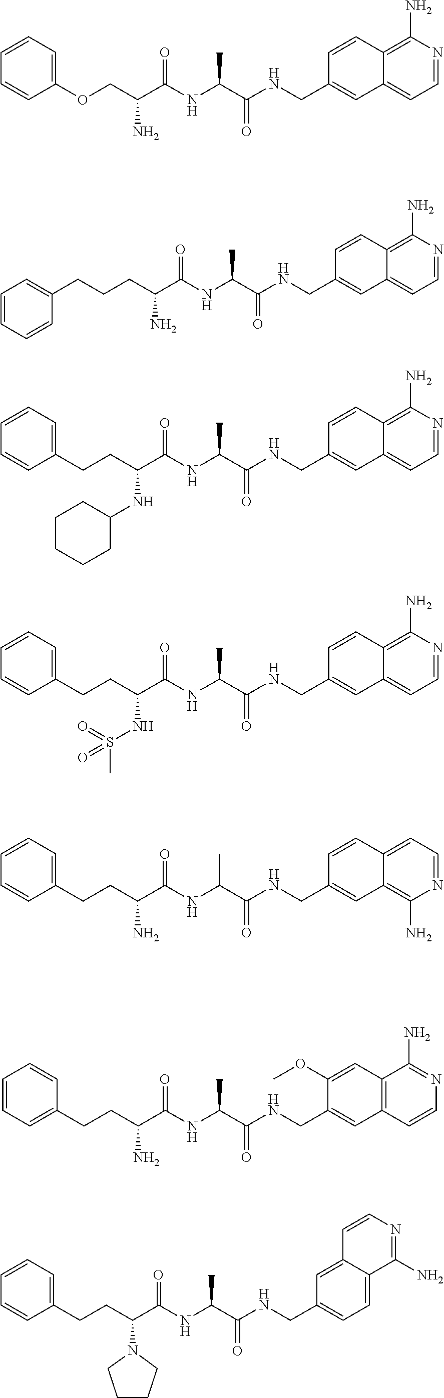 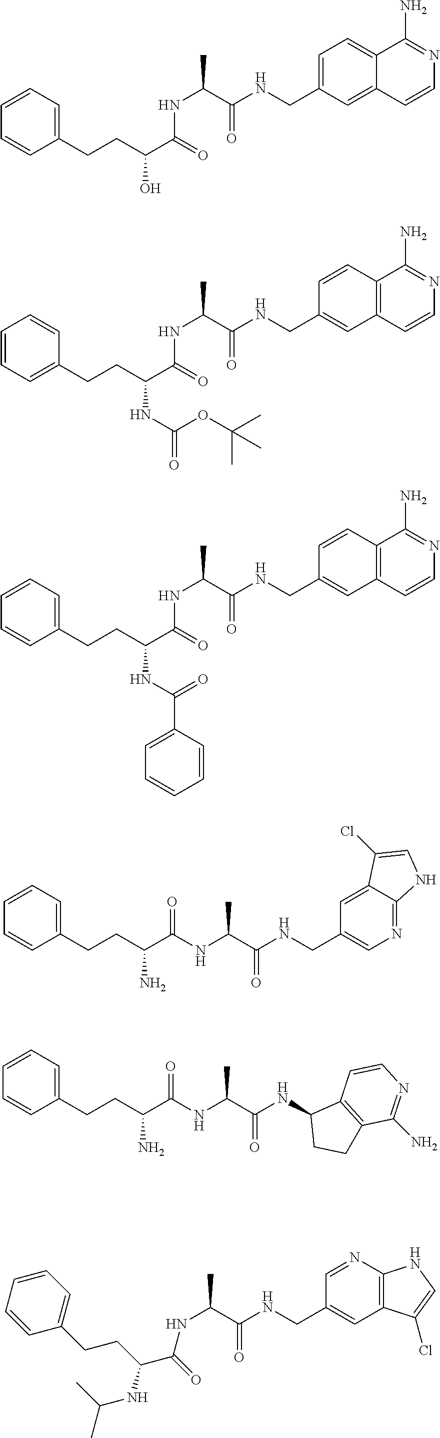 [SMILES: C[C@H](NC(=O)[C@H](O)CCc1ccccc1)C(=O)NCc1ccc2c(N)nccc2c1 ; C[C@H](NC(=O)[C@@H](CCc1ccccc1)NC(=O)OC(C)(C)C)C(=O)NCc1ccc2c(N)nccc2c1 ; C[C@H](NC(=O)[C@@H](CCc1ccccc1)NC(=O)c1ccccc1)C(=O)NCc1ccc2c(N)nccc2c1 ; C[C@H](NC(=O)[C@H](N)CCc1ccccc1)C(=O)NCc1cnc2[nH]cc(Cl)c2c1 ; C[C@H](NC(=O)[C@H](N)CCc1ccccc1)C(=O)N[C@@H]1CCc2c1ccnc2N ; CC(C)N[C@H](CCc1ccccc1)C(=O)N[C@@H](C)C(=O)NCc1cnc2[nH]cc(Cl)c2c1] 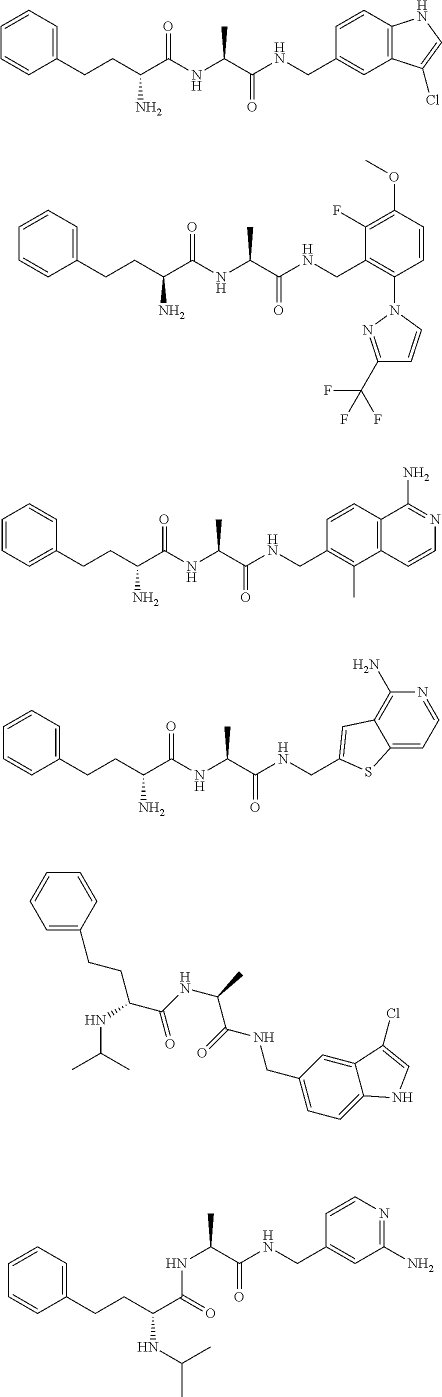 [SMILES: C[C@H](NC(=O)[C@H](N)CCc1ccccc1)C(=O)NCc1ccc2[nH]cc(Cl)c2c1 ; COc1ccc(-n2ccc(C(F)(F)F)n2)c(CNC(=O)[C@H](C)NC(=O)[C@@H](N)CCc2ccccc2)c1F ; Cc1c(CNC(=O)[C@H](C)NC(=O)[C@H](N)CCc2ccccc2)ccc2c(N)nccc12 ; C[C@H](NC(=O)[C@H](N)CCc1ccccc1)C(=O)NCc1cc2c(N)nccc2s1 ; CC(C)N[C@H](CCc1ccccc1)C(=O)N[C@@H](C)C(=O)NCc1ccc2[nH]cc(Cl)c2c1 ; CC(C)N[C@H](CCc1ccccc1)C(=O)N[C@@H](C)C(=O)NCc1ccnc(N)c1] 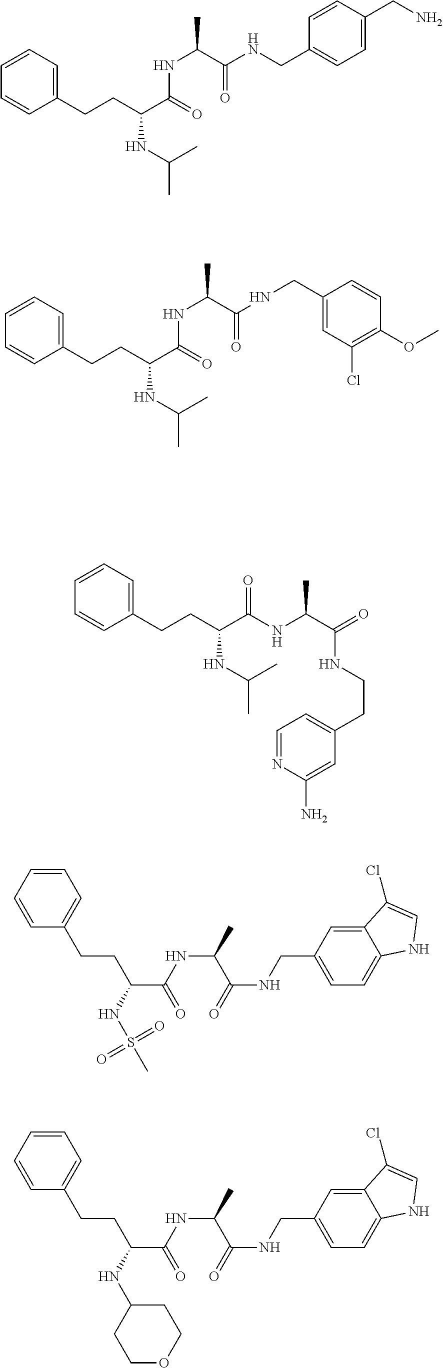 [SMILES: CC(C)N[C@H](CCc1ccccc1)C(=O)N[C@@H](C)C(=O)NCc1ccc(CN)cc1 ; COc1ccc(CNC(=O)[C@H](C)NC(=O)[C@@H](CCc2ccccc2)NC(C)C)cc1Cl ; CC(C)N[C@H](CCc1ccccc1)C(=O)N[C@@H](C)C(=O)NCCc1ccnc(N)c1 ; C[C@H](NC(=O)[C@@H](CCc1ccccc1)NS(C)(=O)=O)C(=O)NCc1ccc2[nH]cc(Cl)c2c1 ; C[C@H](NC(=O)[C@@H](CCc1ccccc1)NC1CCOCC1)C(=O)NCc1ccc2[nH]cc(Cl)c2c1] 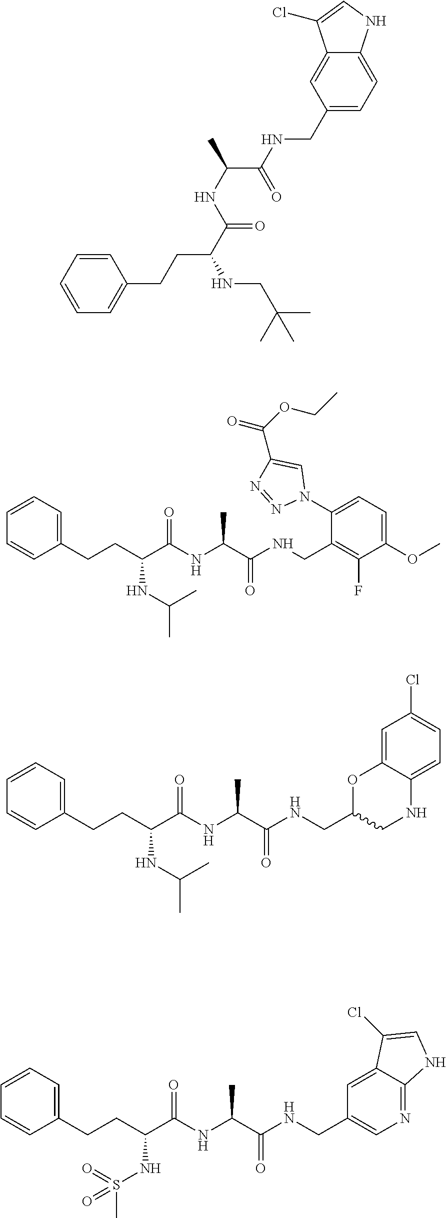 [SMILES: C[C@H](NC(=O)[C@@H](CCc1ccccc1)NCC(C)(C)C)C(=O)NCc1ccc2[nH]cc(Cl)c2c1 ; CCOC(=O)c1cn(-c2ccc(OC)c(F)c2CNC(=O)[C@H](C)NC(=O)[C@@H](CCc2ccccc2)NC(C)C)nn1 ; CC(C)N[C@H](CCc1ccccc1)C(=O)N[C@@H](C)C(=O)NCC1CNc2ccc(Cl)cc2O1 ; C[C@H](NC(=O)[C@@H](CCc1ccccc1)NS(C)(=O)=O)C(=O)NCc1cnc2[nH]cc(Cl)c2c1] 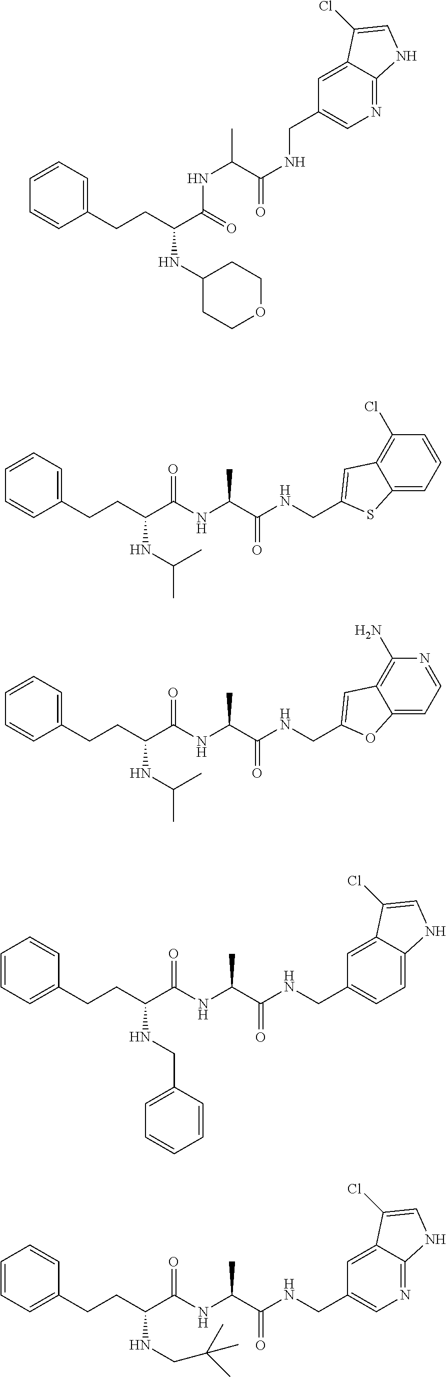 [SMILES: CC(NC(=O)[C@@H](CCc1ccccc1)NC1CCOCC1)C(=O)NCc1cnc2[nH]cc(Cl)c2c1 ; CC(C)N[C@H](CCc1ccccc1)C(=O)N[C@@H](C)C(=O)NCc1cc2c(Cl)cccc2s1 ; CC(C)N[C@H](CCc1ccccc1)C(=O)N[C@@H](C)C(=O)NCc1cc2c(N)nccc2o1 ; C[C@H](NC(=O)[C@@H](CCc1ccccc1)NCc1ccccc1)C(=O)NCc1ccc2[nH]cc(Cl)c2c1 ; C[C@H](NC(=O)[C@@H](CCc1ccccc1)NCC(C)(C)C)C(=O)NCc1cnc2[nH]cc(Cl)c2c1] 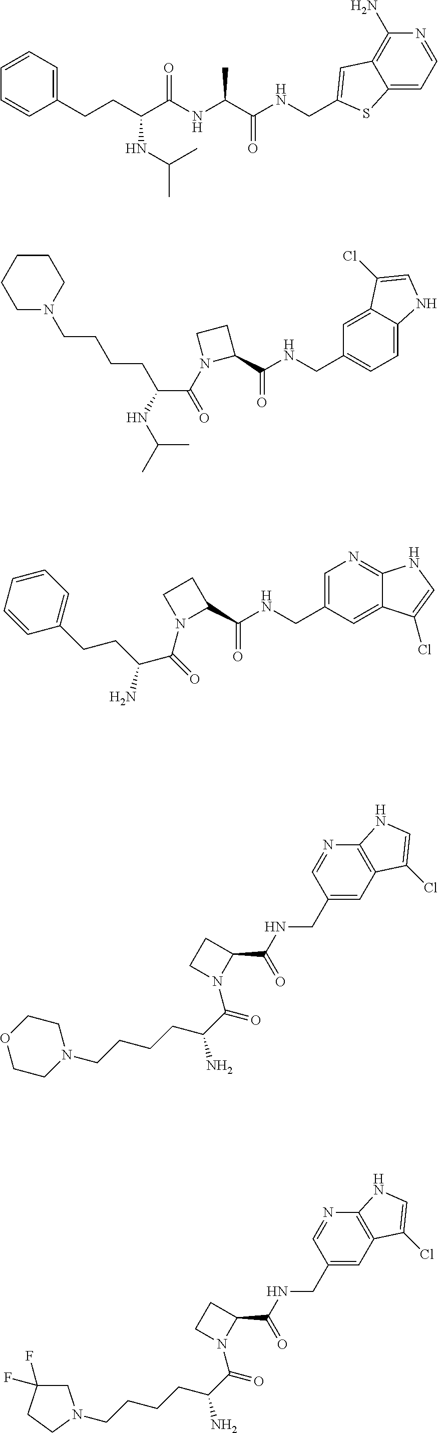 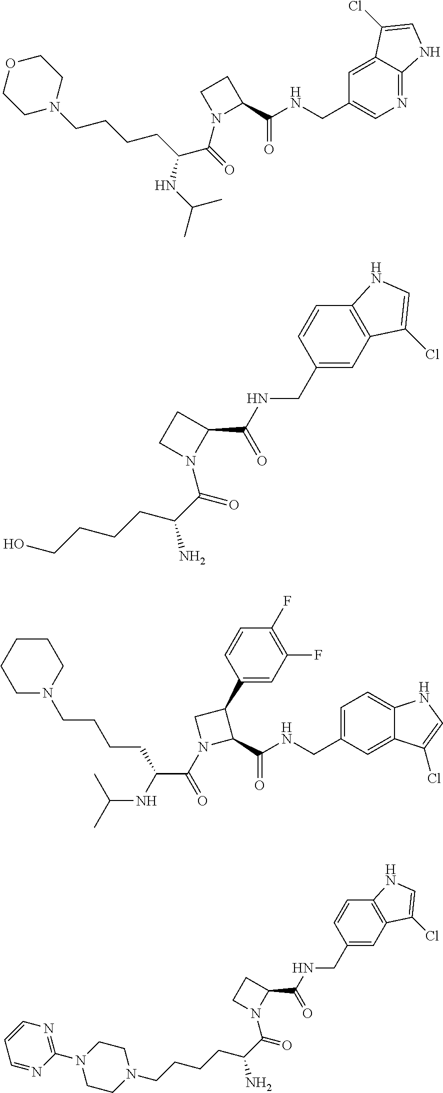 [SMILES: CC(C)N[C@H](CCCCN1CCOCC1)C(=O)N1CC[C@H]1C(=O)NCc1cnc2[nH]cc(Cl)c2c1 ; N[C@H](CCCCO)C(=O)N1CC[C@H]1C(=O)NCc1ccc2[nH]cc(Cl)c2c1 ; CC(C)N[C@H](CCCCN1CCCCC1)C(=O)N1C[C@@H](c2ccc(F)c(F)c2)[C@H]1C(=O)NCc1ccc2[nH]cc(Cl)c2c1 ; N[C@H](CCCCN1CCN(c2ncccn2)CC1)C(=O)N1CC[C@H]1C(=O)NCc1ccc2[nH]cc(Cl)c2c1] 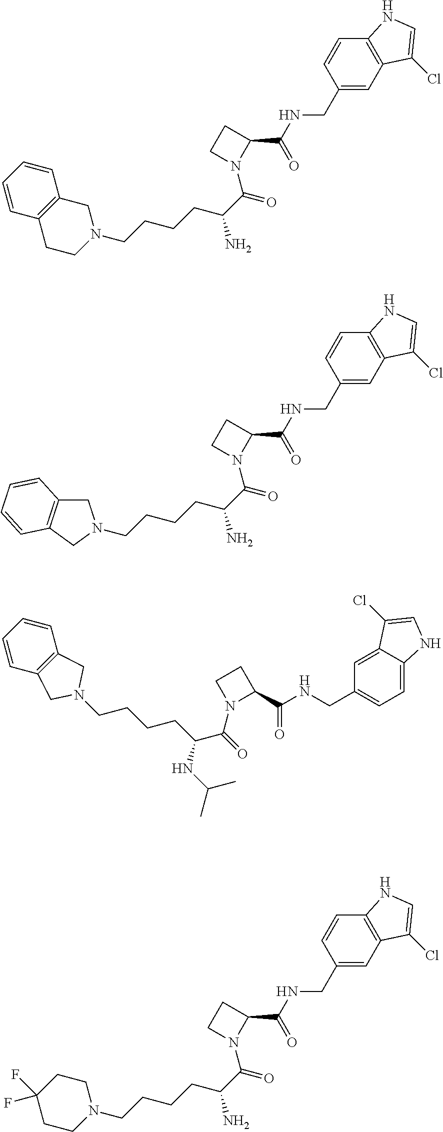 [SMILES: N[C@H](CCCCN1CCc2ccccc2C1)C(=O)N1CC[C@H]1C(=O)NCc1ccc2[nH]cc(Cl)c2c1 ; N[C@H](CCCCN1Cc2ccccc2C1)C(=O)N1CC[C@H]1C(=O)NCc1ccc2[nH]cc(Cl)c2c1 ; CC(C)N[C@H](CCCCN1Cc2ccccc2C1)C(=O)N1CC[C@H]1C(=O)NCc1ccc2[nH]cc(Cl)c2c1 ; N[C@H](CCCCN1CCC(F)(F)CC1)C(=O)N1CC[C@H]1C(=O)NCc1ccc2[nH]cc(Cl)c2c1] 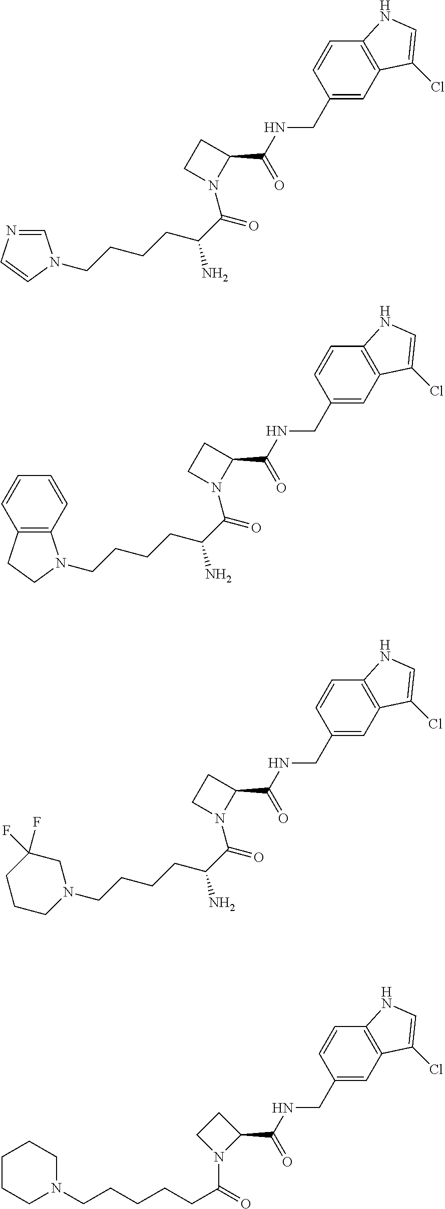 [SMILES: N[C@H](CCCCn1ccnc1)C(=O)N1CC[C@H]1C(=O)NCc1ccc2[nH]cc(Cl)c2c1 ; N[C@H](CCCCN1CCc2ccccc21)C(=O)N1CC[C@H]1C(=O)NCc1ccc2[nH]cc(Cl)c2c1 ; N[C@H](CCCCN1CCCC(F)(F)C1)C(=O)N1CC[C@H]1C(=O)NCc1ccc2[nH]cc(Cl)c2c1 ; O=C(NCc1ccc2[nH]cc(Cl)c2c1)[C@@H]1CCN1C(=O)CCCCCN1CCCCC1] 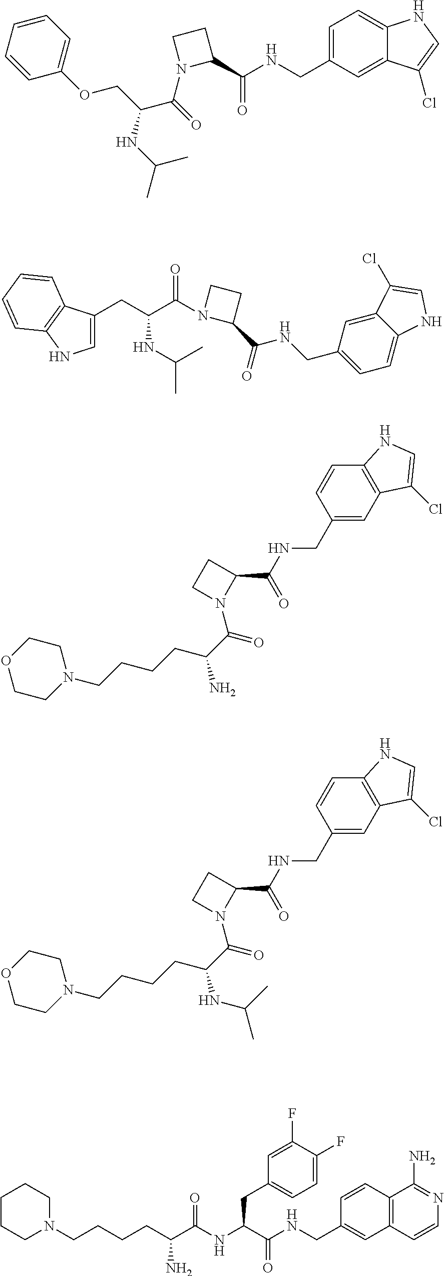 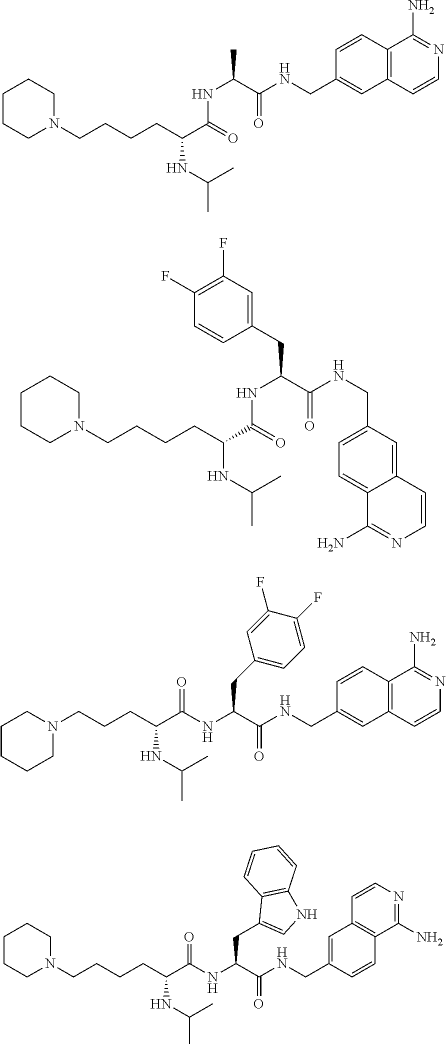 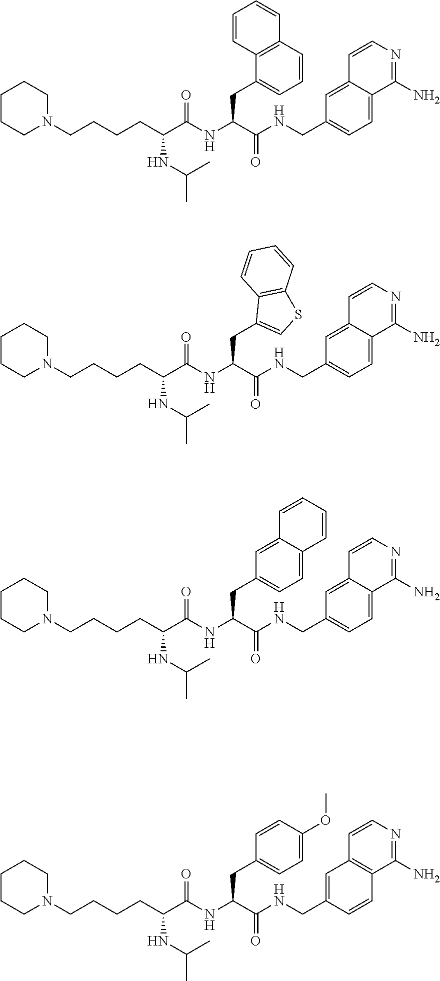 [SMILES: CC(C)N[C@H](CCCCN1CCCCC1)C(=O)N[C@@H](Cc1cccc2ccccc12)C(=O)NCc1ccc2c(N)nccc2c1 ; CC(C)N[C@H](CCCCN1CCCCC1)C(=O)N[C@@H](Cc1csc2ccccc12)C(=O)NCc1ccc2c(N)nccc2c1 ; CC(C)N[C@H](CCCCN1CCCCC1)C(=O)N[C@@H](Cc1ccc2ccccc2c1)C(=O)NCc1ccc2c(N)nccc2c1 ; COc1ccc(C[C@H](NC(=O)[C@@H](CCCCN2CCCCC2)NC(C)C)C(=O)NCc2ccc3c(N)nccc3c2)cc1] 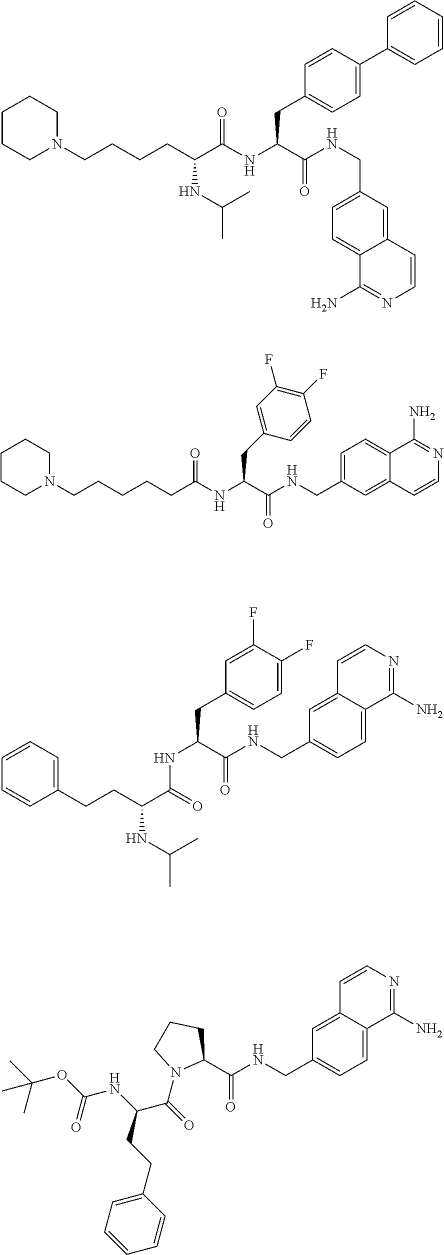 [SMILES: CC(C)N[C@H](CCCCN1CCCCC1)C(=O)N[C@@H](Cc1ccc(-c2ccccc2)cc1)C(=O)NCc1ccc2c(N)nccc2c1 ; Nc1nccc2cc(CNC(=O)[C@H](Cc3ccc(F)c(F)c3)NC(=O)CCCCCN3CCCCC3)ccc12 ; CC(C)N[C@H](CCc1ccccc1)C(=O)N[C@@H](Cc1ccc(F)c(F)c1)C(=O)NCc1ccc2c(N)nccc2c1 ; CC(C)(C)OC(=O)N[C@H](CCc1ccccc1)C(=O)N1CCC[C@H]1C(=O)NCc1ccc2c(N)nccc2c1] 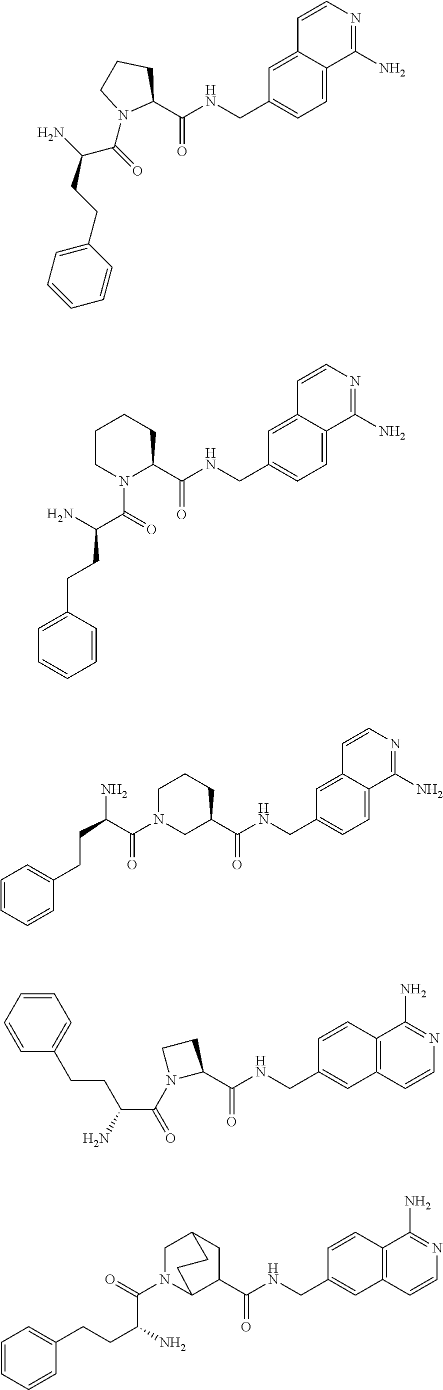 [SMILES: Nc1nccc2cc(CNC(=O)[C@@H]3CCCN3C(=O)[C@H](N)CCc3ccccc3)ccc12 ; Nc1nccc2cc(CNC(=O)[C@@H]3CCCCN3C(=O)[C@H](N)CCc3ccccc3)ccc12 ; Nc1nccc2cc(CNC(=O)[C@@H]3CCCN(C(=O)[C@H](N)CCc4ccccc4)C3)ccc12 ; Nc1nccc2cc(CNC(=O)[C@@H]3CCN3C(=O)[C@H](N)CCc3ccccc3)ccc12 ; Nc1nccc2cc(CNC(=O)C3CC4CCC3N(C(=O)[C@H](N)CCc3ccccc3)C4)ccc12] 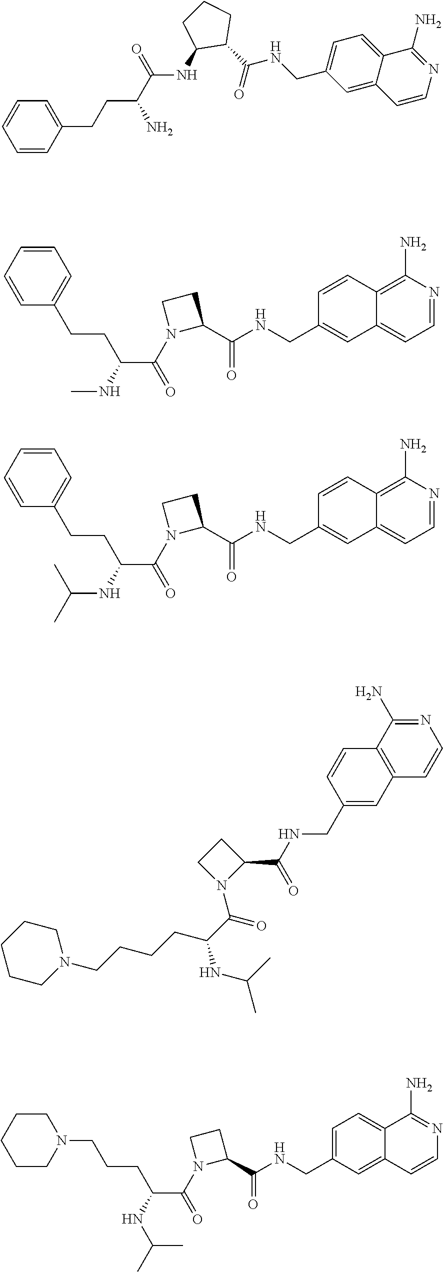 [SMILES: Nc1nccc2cc(CNC(=O)[C@H]3CCC[C@@H]3NC(=O)[C@H](N)CCc3ccccc3)ccc12 ; CN[C@H](CCc1ccccc1)C(=O)N1CC[C@H]1C(=O)NCc1ccc2c(N)nccc2c1 ; CC(C)N[C@H](CCc1ccccc1)C(=O)N1CC[C@H]1C(=O)NCc1ccc2c(N)nccc2c1 ; CC(C)N[C@H](CCCCN1CCCCC1)C(=O)N1CC[C@H]1C(=O)NCc1ccc2c(N)nccc2c1 ; CC(C)N[C@H](CCCN1CCCCC1)C(=O)N1CC[C@H]1C(=O)NCc1ccc2c(N)nccc2c1] 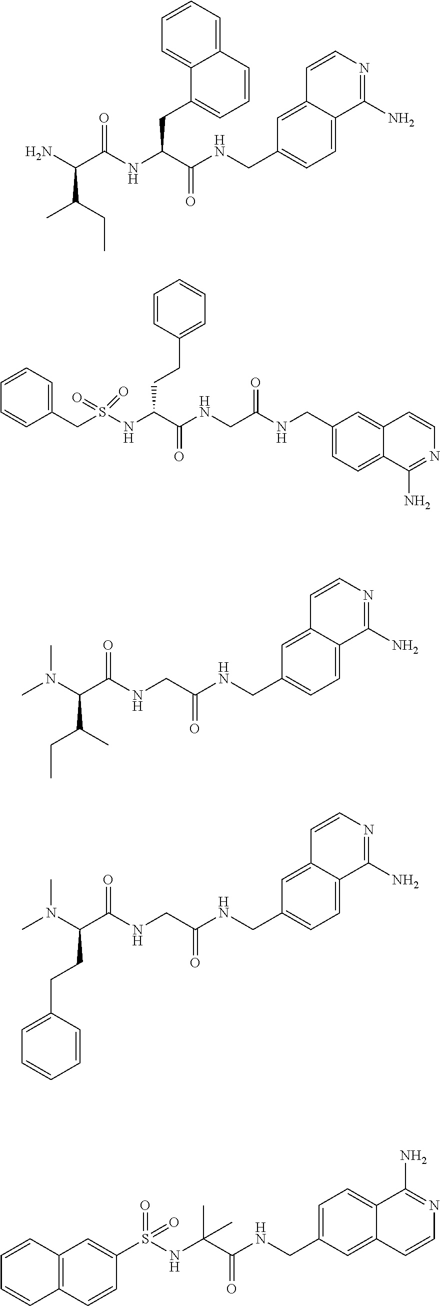 [SMILES: CCC(C)[C@@H](N)C(=O)N[C@@H](Cc1cccc2ccccc12)C(=O)NCc1ccc2c(N)nccc2c1 ; Nc1nccc2cc(CNC(=O)CNC(=O)[C@@H](CCc3ccccc3)NS(=O)(=O)Cc3ccccc3)ccc12 ; CCC(C)[C@H](C(=O)NCC(=O)NCc1ccc2c(N)nccc2c1)N(C)C ; CN(C)[C@H](CCc1ccccc1)C(=O)NCC(=O)NCc1ccc2c(N)nccc2c1 ; CC(C)(NS(=O)(=O)c1ccc2ccccc2c1)C(=O)NCc1ccc2c(N)nccc2c1] 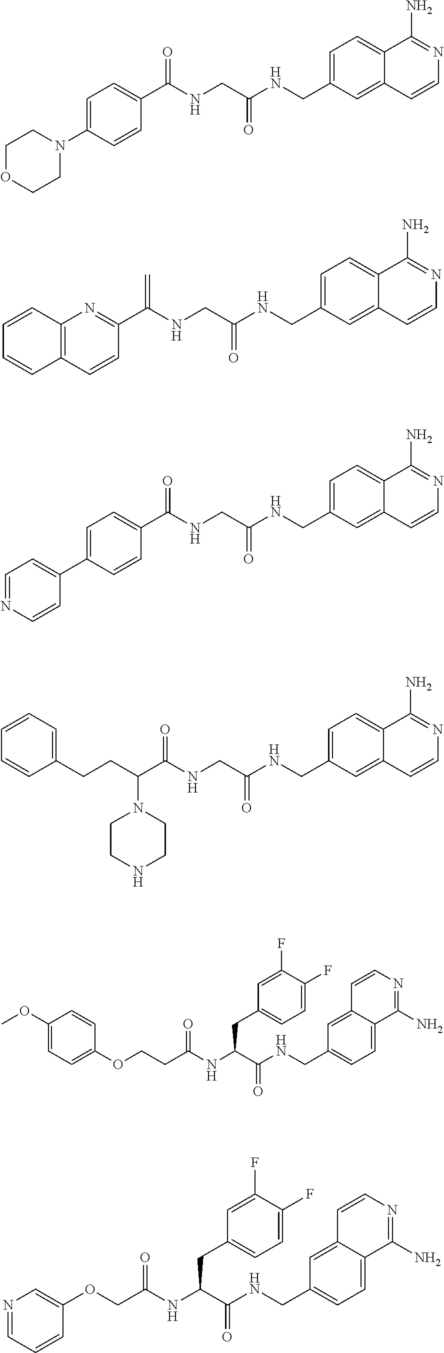 [SMILES: Nc1nccc2cc(CNC(=O)CNC(=O)c3ccc(N4CCOCC4)cc3)ccc12 ; C=C(NCC(=O)NCc1ccc2c(N)nccc2c1)c1ccc2ccccc2n1 ; Nc1nccc2cc(CNC(=O)CNC(=O)c3ccc(-c4ccncc4)cc3)ccc12 ; Nc1nccc2cc(CNC(=O)CNC(=O)C(CCc3ccccc3)N3CCNCC3)ccc12 ; COc1ccc(OCCC(=O)N[C@@H](Cc2ccc(F)c(F)c2)C(=O)NCc2ccc3c(N)nccc3c2)cc1 ; Nc1nccc2cc(CNC(=O)[C@H](Cc3ccc(F)c(F)c3)NC(=O)COc3cccnc3)ccc12] 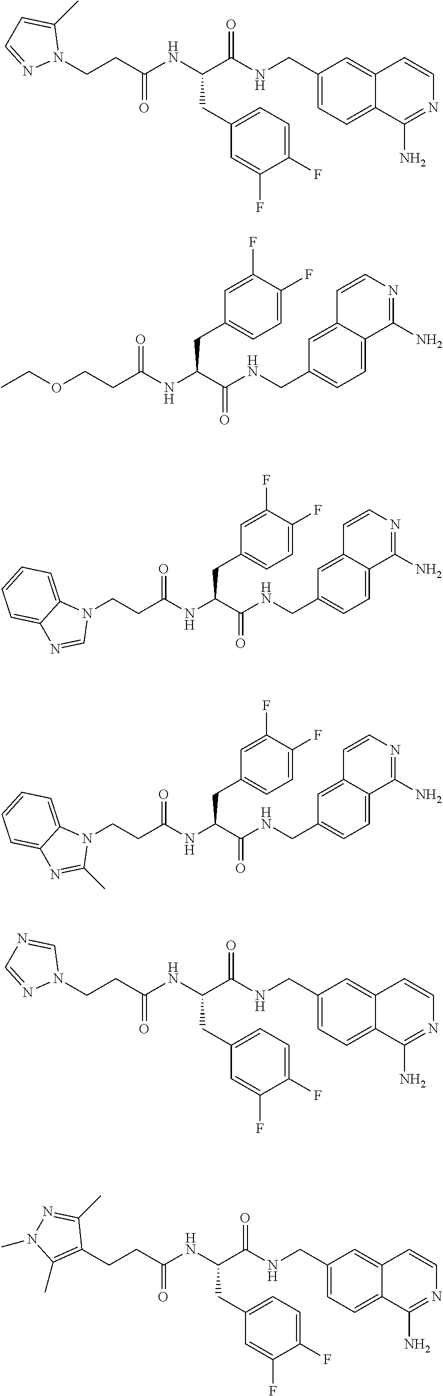 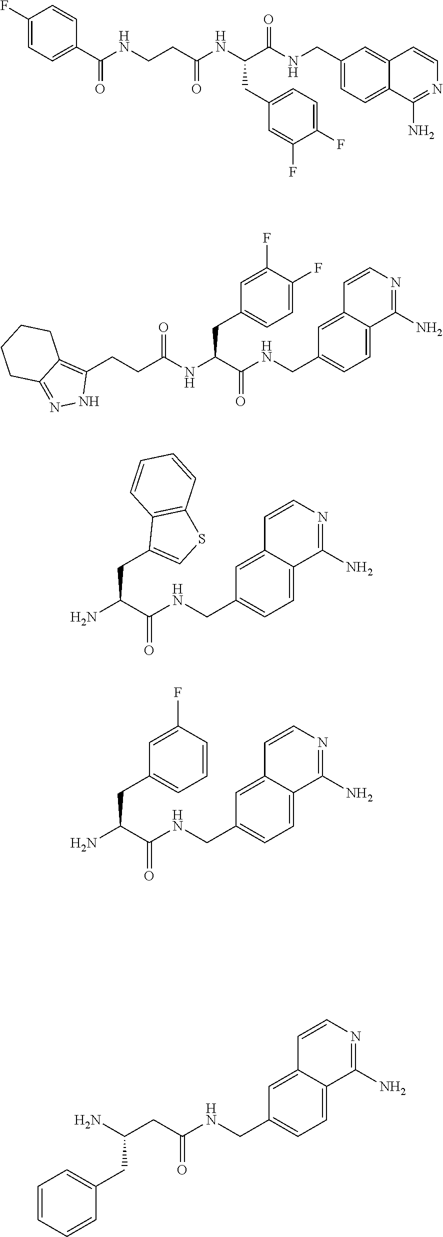 [SMILES: Nc1nccc2cc(CNC(=O)[C@H](Cc3ccc(F)c(F)c3)NC(=O)CCNC(=O)c3ccc(F)cc3)ccc12 ; Nc1nccc2cc(CNC(=O)[C@H](Cc3ccc(F)c(F)c3)NC(=O)CCc3[nH]nc4c3CCCC4)ccc12 ; Nc1nccc2cc(CNC(=O)[C@@H](N)Cc3csc4ccccc34)ccc12 ; Nc1nccc2cc(CNC(=O)[C@@H](N)Cc3cccc(F)c3)ccc12 ; Nc1nccc2cc(CNC(=O)C[C@@H](N)Cc3ccccc3)ccc12] 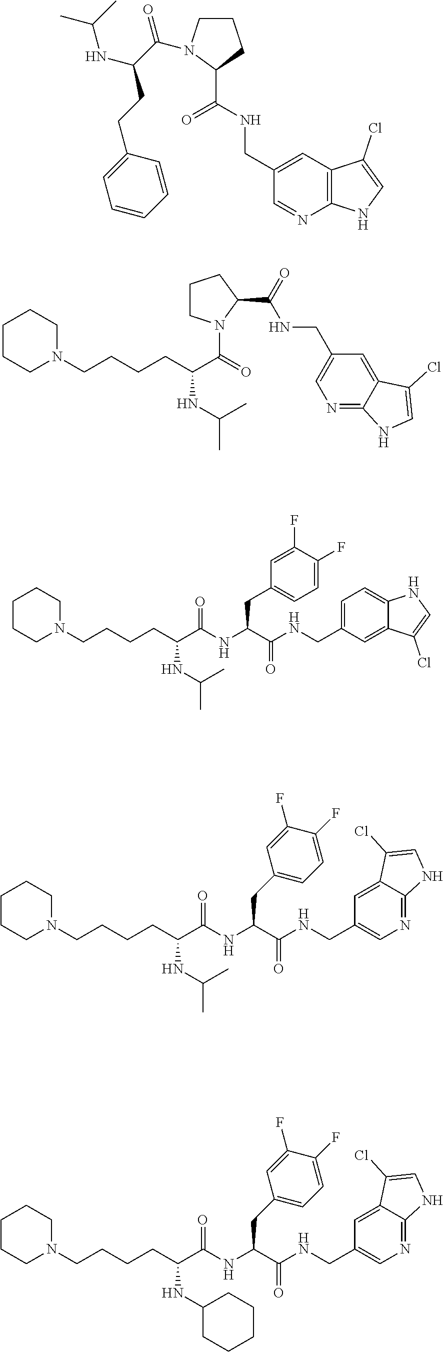 [SMILES: CC(C)N[C@H](CCc1ccccc1)C(=O)N1CCC[C@H]1C(=O)NCc1cnc2[nH]cc(Cl)c2c1 ; CC(C)N[C@H](CCCCN1CCCCC1)C(=O)N1CCC[C@H]1C(=O)NCc1cnc2[nH]cc(Cl)c2c1 ; CC(C)N[C@H](CCCCN1CCCCC1)C(=O)N[C@@H](Cc1ccc(F)c(F)c1)C(=O)NCc1ccc2[nH]cc(Cl)c2c1 ; CC(C)N[C@H](CCCCN1CCCCC1)C(=O)N[C@@H](Cc1ccc(F)c(F)c1)C(=O)NCc1cnc2[nH]cc(Cl)c2c1 ; O=C(NCc1cnc2[nH]cc(Cl)c2c1)[C@H](Cc1ccc(F)c(F)c1)NC(=O)[C@@H](CCCCN1CCCCC1)NC1CCCCC1] 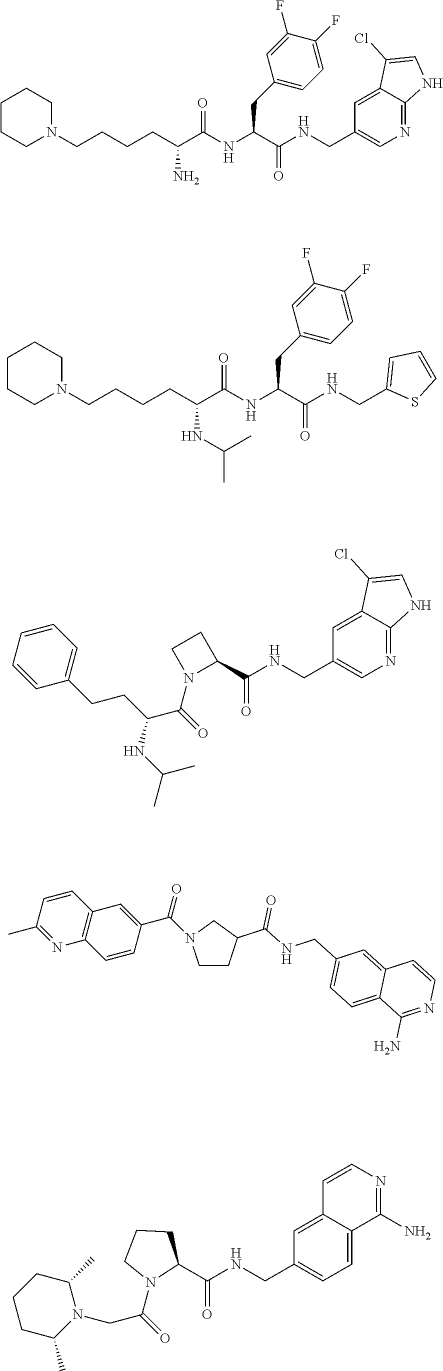 [SMILES: N[C@H](CCCCN1CCCCC1)C(=O)N[C@@H](Cc1ccc(F)c(F)c1)C(=O)NCc1cnc2[nH]cc(Cl)c2c1 ; CC(C)N[C@H](CCCCN1CCCCC1)C(=O)N[C@@H](Cc1ccc(F)c(F)c1)C(=O)NCc1cccs1 ; CC(C)N[C@H](CCc1ccccc1)C(=O)N1CC[C@H]1C(=O)NCc1cnc2[nH]cc(Cl)c2c1 ; Cc1ccc2cc(C(=O)N3CCC(C(=O)NCc4ccc5c(N)nccc5c4)C3)ccc2n1 ; C[C@@H]1CCC[C@H](C)N1CC(=O)N1CCC[C@H]1C(=O)NCc1ccc2c(N)nccc2c1] 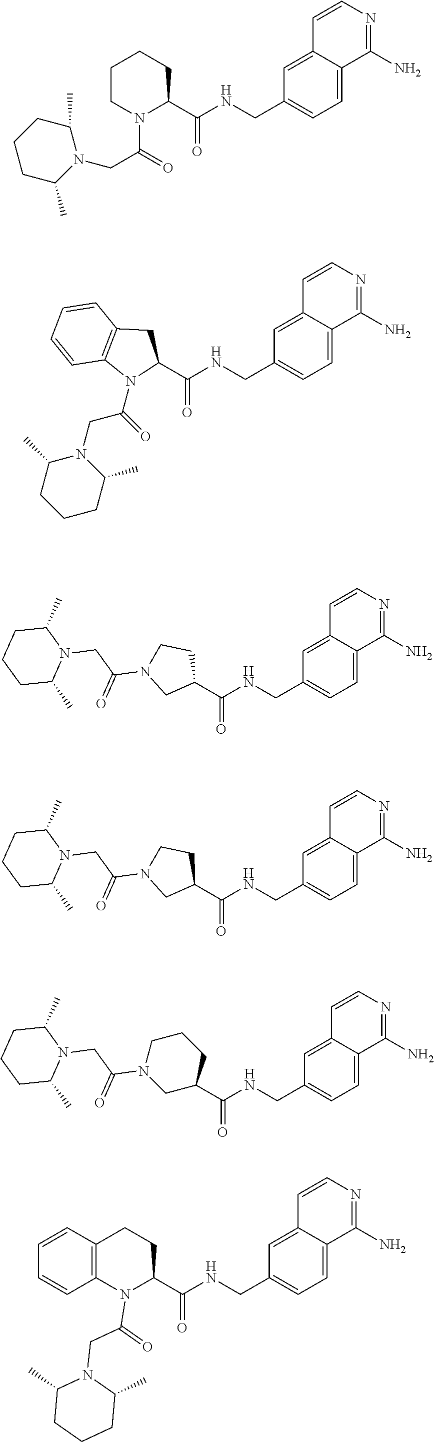 [SMILES: C[C@@H]1CCC[C@H](C)N1CC(=O)N1CCCC[C@H]1C(=O)NCc1ccc2c(N)nccc2c1 ; C[C@@H]1CCC[C@H](C)N1CC(=O)N1c2ccccc2C[C@H]1C(=O)NCc1ccc2c(N)nccc2c1 ; C[C@@H]1CCC[C@H](C)N1CC(=O)N1CC[C@H](C(=O)NCc2ccc3c(N)nccc3c2)C1 ; C[C@@H]1CCC[C@H](C)N1CC(=O)N1CC[C@@H](C(=O)NCc2ccc3c(N)nccc3c2)C1 ; C[C@@H]1CCC[C@H](C)N1CC(=O)N1CCC[C@@H](C(=O)NCc2ccc3c(N)nccc3c2)C1 ; C[C@@H]1CCC[C@H](C)N1CC(=O)N1c2ccccc2CC[C@H]1C(=O)NCc1ccc2c(N)nccc2c1] 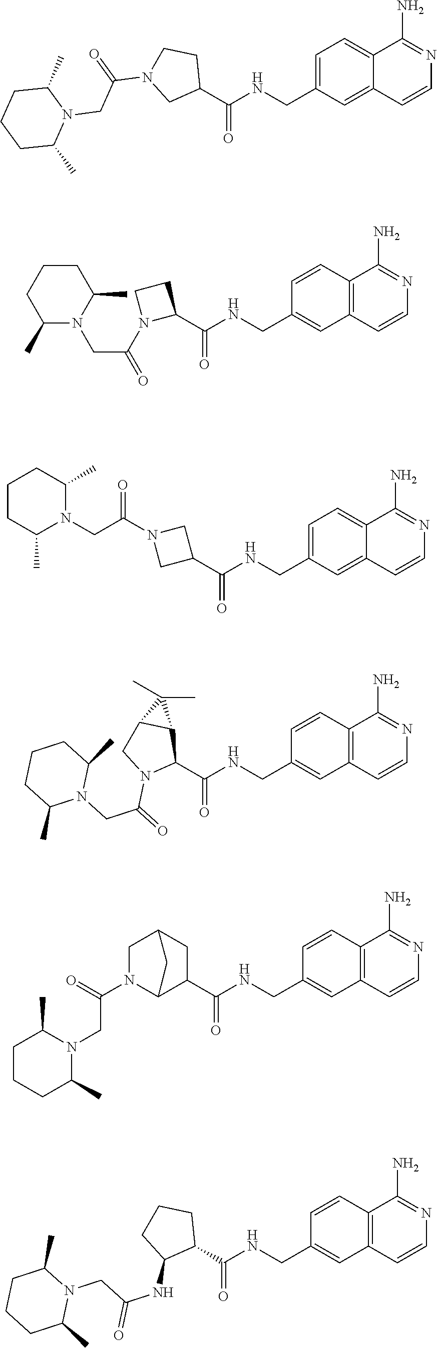 [SMILES: C[C@@H]1CCC[C@H](C)N1CC(=O)N1CCC(C(=O)NCc2ccc3c(N)nccc3c2)C1 ; C[C@@H]1CCC[C@H](C)N1CC(=O)N1CC[C@H]1C(=O)NCc1ccc2c(N)nccc2c1 ; C[C@@H]1CCC[C@H](C)N1CC(=O)N1CC(C(=O)NCc2ccc3c(N)nccc3c2)C1 ; C[C@@H]1CCC[C@H](C)N1CC(=O)N1C[C@H]2[C@@H]([C@H]1C(=O)NCc1ccc3c(N)nccc3c1)C2(C)C ; C[C@@H]1CCC[C@H](C)N1CC(=O)N1CC2CC(C(=O)NCc3ccc4c(N)nccc4c3)C1C2 ; C[C@@H]1CCC[C@H](C)N1CC(=O)N[C@H]1CCC[C@@H]1C(=O)NCc1ccc2c(N)nccc2c1] 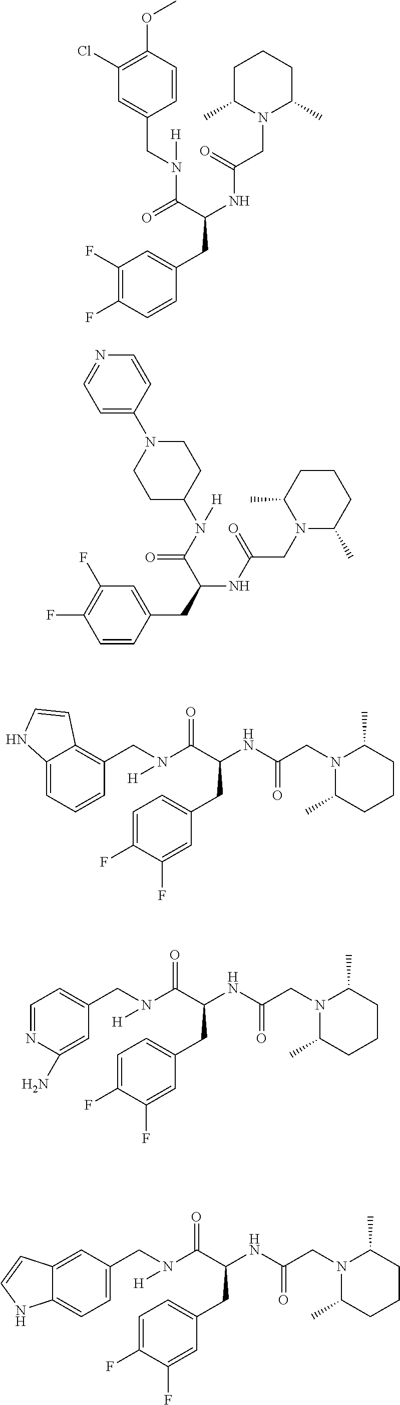 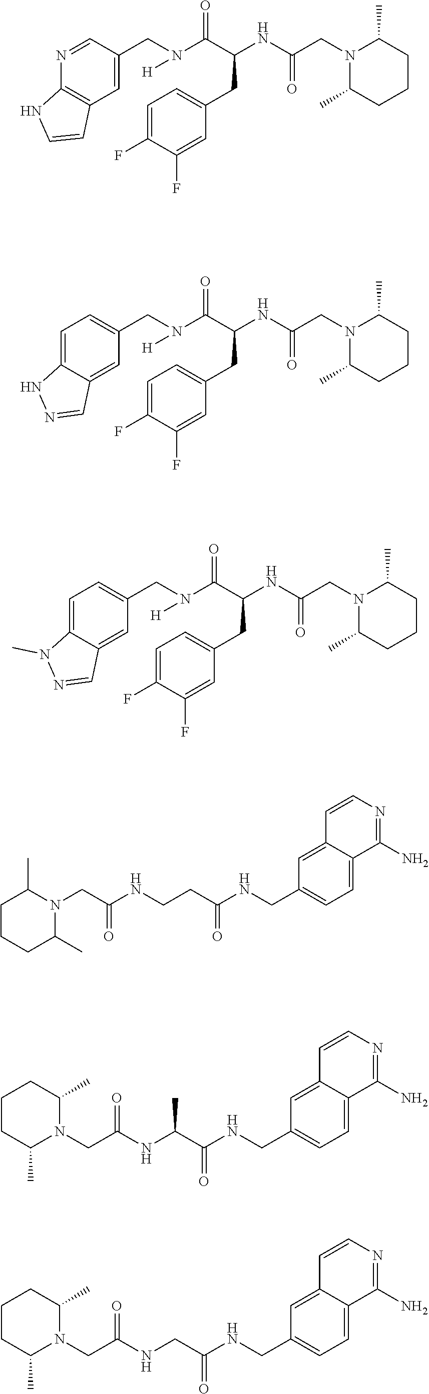 [SMILES: C[C@@H]1CCC[C@H](C)N1CC(=O)N[C@@H](Cc1ccc(F)c(F)c1)C(=O)NCc1cnc2[nH]ccc2c1 ; C[C@@H]1CCC[C@H](C)N1CC(=O)N[C@@H](Cc1ccc(F)c(F)c1)C(=O)NCc1ccc2[nH]ncc2c1 ; C[C@@H]1CCC[C@H](C)N1CC(=O)N[C@@H](Cc1ccc(F)c(F)c1)C(=O)NCc1ccc2c(cnn2C)c1 ; CC1CCCC(C)N1CC(=O)NCCC(=O)NCc1ccc2c(N)nccc2c1 ; C[C@H](NC(=O)CN1[C@H](C)CCC[C@@H]1C)C(=O)NCc1ccc2c(N)nccc2c1 ; C[C@@H]1CCC[C@H](C)N1CC(=O)NCC(=O)NCc1ccc2c(N)nccc2c1] 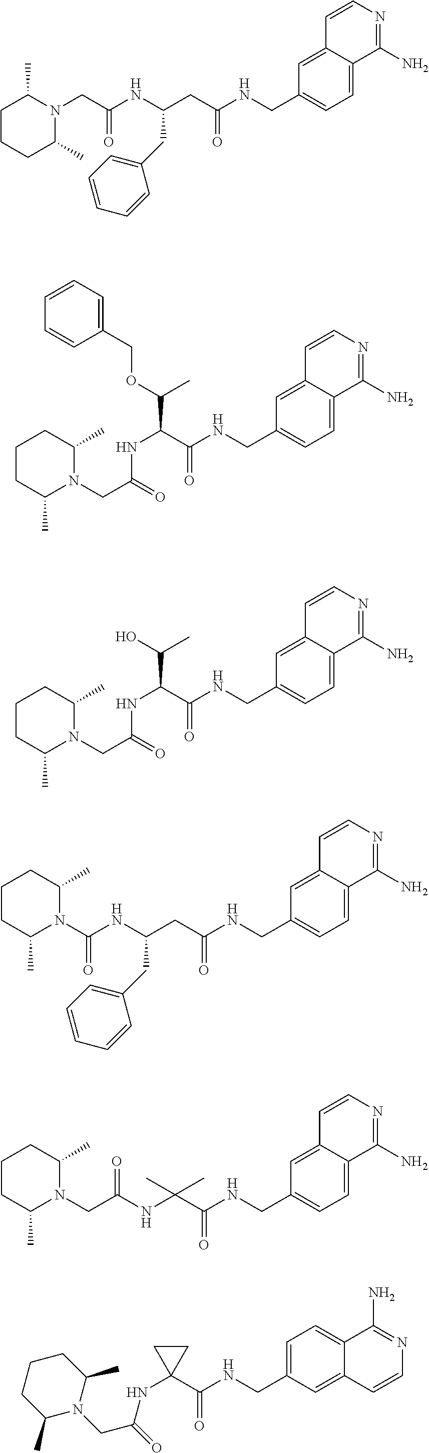 [SMILES: C[C@@H]1CCC[C@H](C)N1CC(=O)N[C@H](CC(=O)NCc1ccc2c(N)nccc2c1)Cc1ccccc1 ; CC(OCc1ccccc1)[C@H](NC(=O)CN1[C@H](C)CCC[C@@H]1C)C(=O)NCc1ccc2c(N)nccc2c1 ; CC(O)[C@H](NC(=O)CN1[C@H](C)CCC[C@@H]1C)C(=O)NCc1ccc2c(N)nccc2c1 ; C[C@@H]1CCC[C@H](C)N1C(=O)N[C@H](CC(=O)NCc1ccc2c(N)nccc2c1)Cc1ccccc1 ; C[C@@H]1CCC[C@H](C)N1CC(=O)NC(C)(C)C(=O)NCc1ccc2c(N)nccc2c1 ; C[C@@H]1CCC[C@H](C)N1CC(=O)NC1(C(=O)NCc2ccc3c(N)nccc3c2)CC1] 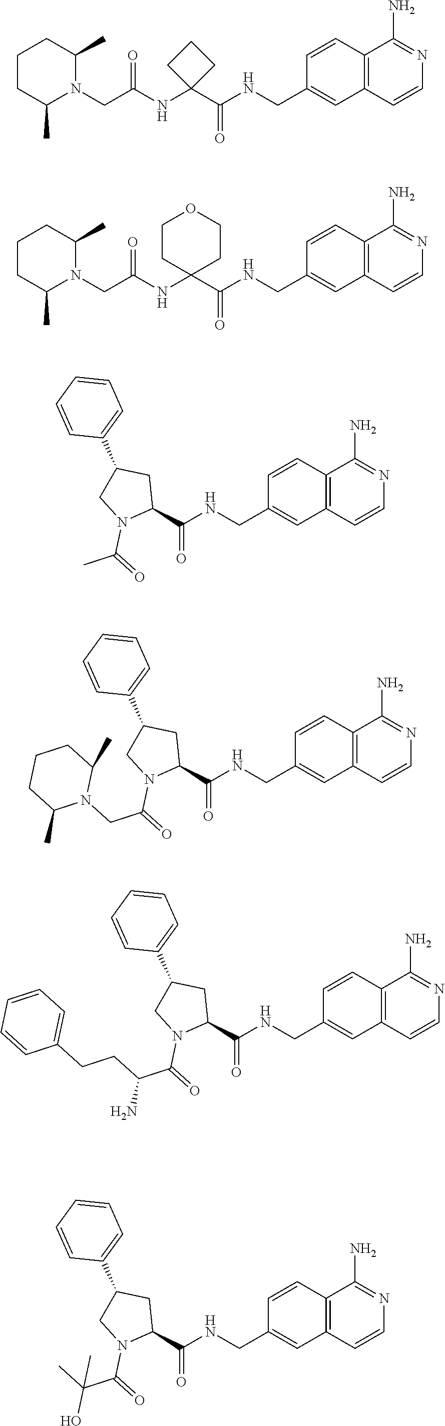 [SMILES: C[C@@H]1CCC[C@H](C)N1CC(=O)NC1(C(=O)NCc2ccc3c(N)nccc3c2)CCC1 ; C[C@@H]1CCC[C@H](C)N1CC(=O)NC1(C(=O)NCc2ccc3c(N)nccc3c2)CCOCC1 ; CC(=O)N1C[C@H](c2ccccc2)C[C@H]1C(=O)NCc1ccc2c(N)nccc2c1 ; C[C@@H]1CCC[C@H](C)N1CC(=O)N1C[C@H](c2ccccc2)C[C@H]1C(=O)NCc1ccc2c(N)nccc2c1 ; Nc1nccc2cc(CNC(=O)[C@@H]3C[C@@H](c4ccccc4)CN3C(=O)[C@H](N)CCc3ccccc3)ccc12 ; CC(C)(O)C(=O)N1C[C@H](c2ccccc2)C[C@H]1C(=O)NCc1ccc2c(N)nccc2c1] 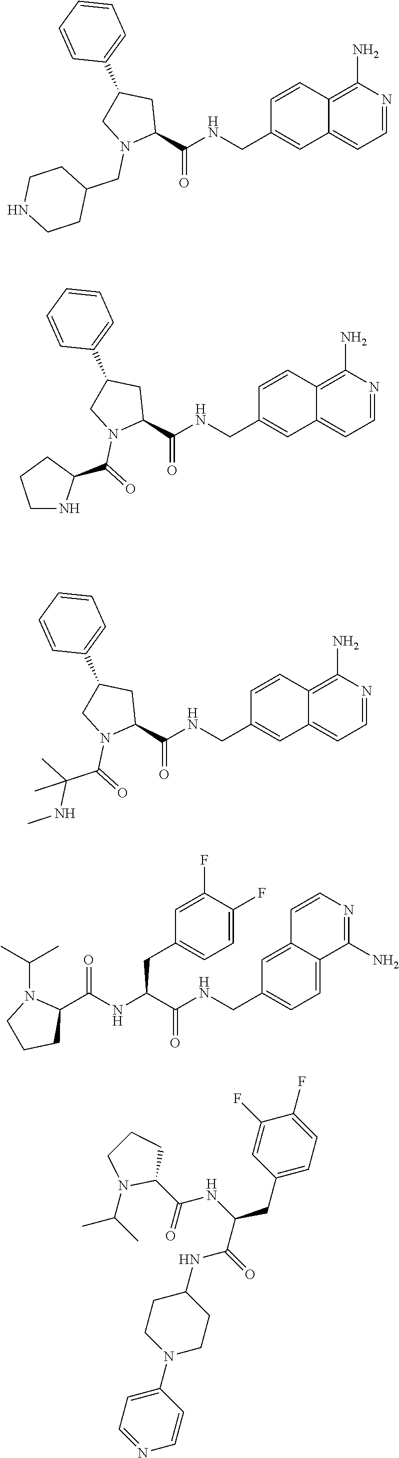 [SMILES: Nc1nccc2cc(CNC(=O)[C@@H]3C[C@@H](c4ccccc4)CN3CC3CCNCC3)ccc12 ; Nc1nccc2cc(CNC(=O)[C@@H]3C[C@@H](c4ccccc4)CN3C(=O)[C@@H]3CCCN3)ccc12 ; CNC(C)(C)C(=O)N1C[C@H](c2ccccc2)C[C@H]1C(=O)NCc1ccc2c(N)nccc2c1 ; CC(C)N1CCC[C@@H]1C(=O)N[C@@H](Cc1ccc(F)c(F)c1)C(=O)NCc1ccc2c(N)nccc2c1 ; CC(C)N1CCC[C@@H]1C(=O)N[C@@H](Cc1ccc(F)c(F)c1)C(=O)NC1CCN(c2ccncc2)CC1] 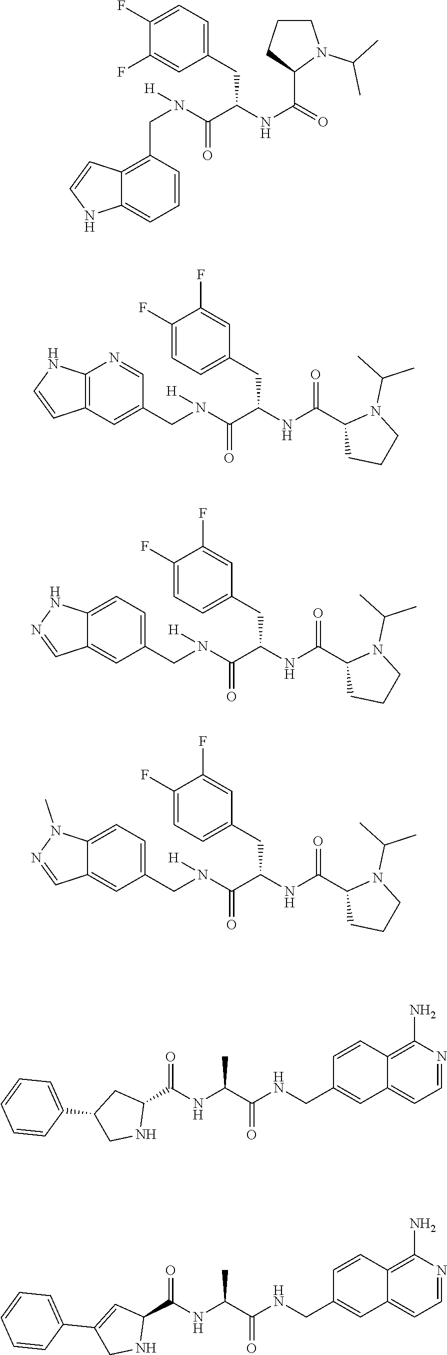 [SMILES: CC(C)N1CCC[C@@H]1C(=O)N[C@@H](Cc1ccc(F)c(F)c1)C(=O)NCc1cccc2[nH]ccc12 ; CC(C)N1CCC[C@@H]1C(=O)N[C@@H](Cc1ccc(F)c(F)c1)C(=O)NCc1cnc2[nH]ccc2c1 ; CC(C)N1CCC[C@@H]1C(=O)N[C@@H](Cc1ccc(F)c(F)c1)C(=O)NCc1ccc2[nH]ncc2c1 ; CC(C)N1CCC[C@@H]1C(=O)N[C@@H](Cc1ccc(F)c(F)c1)C(=O)NCc1ccc2c(cnn2C)c1 ; C[C@H](NC(=O)[C@H]1C[C@@H](c2ccccc2)CN1)C(=O)NCc1ccc2c(N)nccc2c1 ; C[C@H](NC(=O)[C@@H]1C=C(c2ccccc2)CN1)C(=O)NCc1ccc2c(N)nccc2c1] 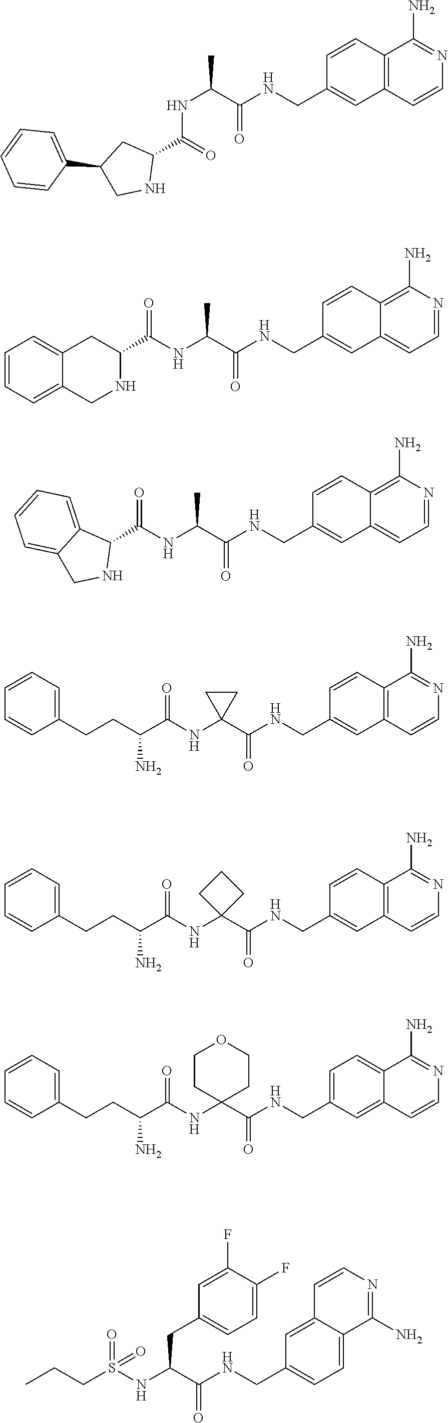 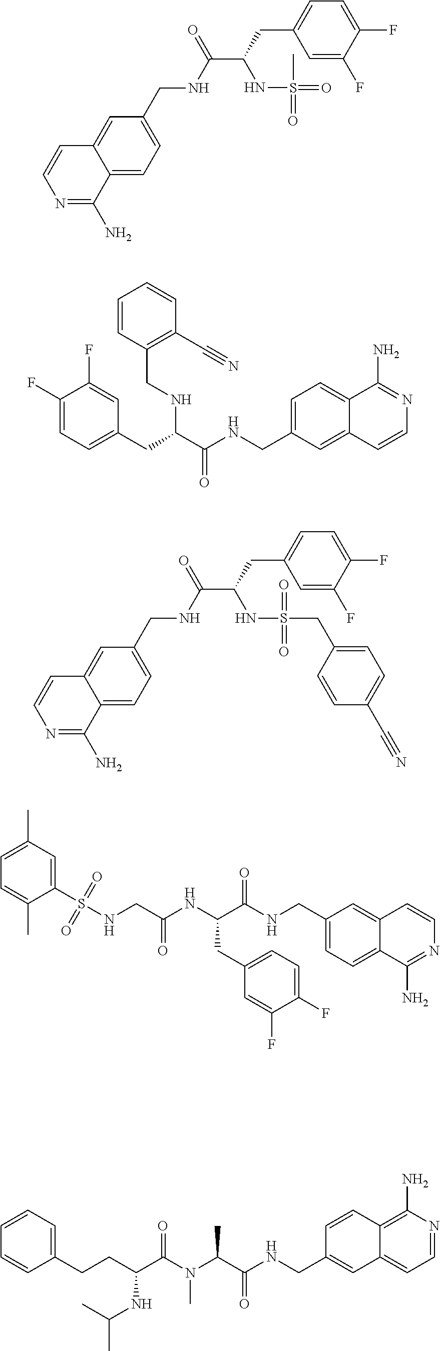 [SMILES: CS(=O)(=O)N[C@@H](Cc1ccc(F)c(F)c1)C(=O)NCc1ccc2c(N)nccc2c1 ; N#Cc1ccccc1CN[C@@H](Cc1ccc(F)c(F)c1)C(=O)NCc1ccc2c(N)nccc2c1 ; N#Cc1ccc(CS(=O)(=O)N[C@@H](Cc2ccc(F)c(F)c2)C(=O)NCc2ccc3c(N)nccc3c2)cc1 ; Cc1ccc(C)c(S(=O)(=O)NCC(=O)N[C@@H](Cc2ccc(F)c(F)c2)C(=O)NCc2ccc3c(N)nccc3c2)c1 ; CC(C)N[C@H](CCc1ccccc1)C(=O)N(C)[C@@H](C)C(=O)NCc1ccc2c(N)nccc2c1] 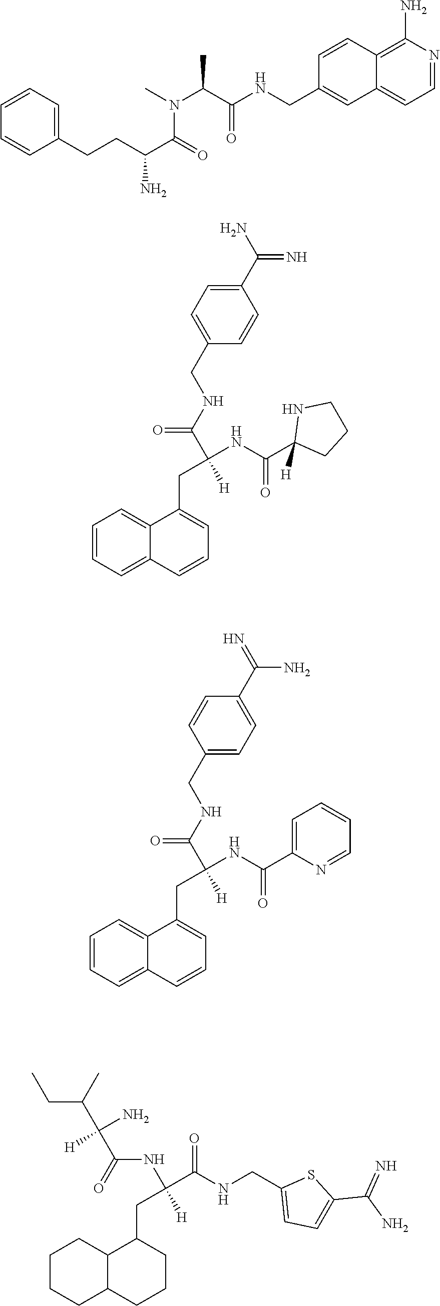 [SMILES: C[C@@H](C(=O)NCc1ccc2c(N)nccc2c1)N(C)C(=O)[C@H](N)CCc1ccccc1 ; N=C(N)c1ccc(CNC(=O)[C@H](Cc2cccc3ccccc23)NC(=O)[C@H]2CCCN2)cc1 ; N=C(N)c1ccc(CNC(=O)[C@H](Cc2cccc3ccccc23)NC(=O)c2ccccn2)cc1 ; CCC(C)[C@@H](N)C(=O)N[C@H](CC1CCCC2CCCCC21)C(=O)NCc1ccc(C(=N)N)s1] 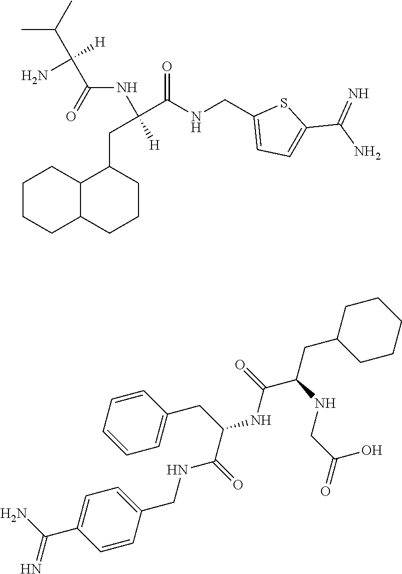 [SMILES: CC(C)[C@H](N)C(=O)N[C@H](CC1CCCC2CCCCC21)C(=O)NCc1ccc(C(=N)N)s1 ; N=C(N)c1ccc(CNC(=O)[C@H](Cc2ccccc2)NC(=O)[C@@H](CC2CCCCC2)NCC(=O)O)cc1]
or pharmaceutically acceptable salt and/or solvate thereof.

49. A pharmaceutical composition comprising: the compound, or a pharmaceutically acceptable salt and/or solvate thereof, according to claim 1, and at least one pharmaceutically acceptable excipient.

50. (canceled)

51. (canceled)

52. A method of treating a disease or condition in which Factor XIIa activity is implicated, comprising administering to a subject in need thereof a therapeutically effective amount of a compound, or a pharmaceutically acceptable salt and/or solvate thereof, of claim 1.

53. (canceled)

54. The method of claim 52, wherein the disease or condition in which Factor XIIa activity is implicated is a bradykinin-mediated angioedema.

55. The method of claim 54, wherein the bradykinin-mediated angioedema is hereditary angioedema.

56. The method of claim 54, wherein the bradykinin-mediated angioedema is non hereditary.

57. The method of claim 52, wherein the disease or condition in which Factor XIIa activity is implicated is selected from vascular hyperpermeability; stroke including ischemic stroke and haemorrhagic accidents; retinal edema; diabetic retinopathy; DME; retinal vein occlusion; or AMD.

58. The method of claim 52, wherein the disease or condition in which Factor XIIa activity is implicated is a thrombotic disorder.

59. The method of claim 58, wherein the thrombotic disorder is thrombosis; thromboembolism caused by increased propensity of medical devices that come into contact with blood to clot blood; prothrombotic conditions such as disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.

60. The method of claim 52, wherein the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; or anaphylaxis.

61. The method of claim 52, wherein the compound targets FXIIa.

62. The compound of claim 1, wherein the stereoisomer is an enantiomer, diastereoisomer, racemic mixture, or scalemic mixture.
